(19) United States
(12) Patent Application Publication
(10) Pub. No.: US 2024/0059798 A1
(43) Pub. Date: Feb. 22, 2024

(54) CONDITIONALLY ACTIVATED ANTIGEN BINDING POLYPEPTIDE COMPLEXES AND METHODS OF USE THEREOF

(71) Applicant: ModeX Therapeutics, Inc., NATICK, MA (US)

(72) Inventors: LING XU, NEWTON CENTER, MA (US); LAN WU, CHESTNUT HILL, MA (US); EDWARD SEUNG, MEDFORD, MA (US); ZHI-YONG YANG, NEWTON CENTER, MA (US); GARY J. NABEL, DELRAY BEACH, FL (US); ELIAS ZERHOUNI, FORT LAUDERDALE, FL (US); RONNIE R. WEI, WESTON, MA (US); HAO CHEN, ACTON, MA (US); MARK GRECI, NATICK, MA (US); NICHOLAS JONES, NATICK, MA (US)

(21) Appl. No.: 18/069,529

(22) Filed: Dec. 21, 2022

Related U.S. Application Data
(60) Provisional application No. 63/292,382, filed on Dec. 21, 2021.

(51) Int. Cl. C07K 16/46; A61P 35/00

(52) U.S. Cl. CPC C07K 16/468; A61P 35/00; C07K 2317/524; C07K 2317/526; C07K 2317/522; C07K 2317/31; C07K 2317/565

(57) ABSTRACT

Disclosed are antigen binding polypeptide complexes (e.g., antibodies and antigen binding fragments thereof) having certain structural features. Also disclosed are polynucleotides and vectors encoding such polypeptide complexes; cells, pharmaceutical compositions, and kits containing such polypeptide complexes; and methods of using such polypeptide complexes.

### CROSS REFERENCE TO RELATED APPLICATION

This application claims the priority benefit of U.S. Provisional Application No. 63/292,382, filed Dec. 21, 2021, which is incorporated herein by reference in its entirety.

### REFERENCE TO SEQUENCE LISTING SUBMITTED ELECTRONICALLY

The content of the electronically submitted sequence listing (Name: 4850_0070001_SequenceListing_ST26; Size: 1,326,349 bytes; and Date of Creation: Dec. 20, 2022) is herein incorporated by reference in its entirety.

### FIELD

The present disclosure relates to antigen binding polypeptide complexes (e.g., antibodies and antigen binding fragments thereof) having certain structural features. The present disclosure also relates to conditionally activated antigen binding polypeptide complexes (e.g., antibodies and antigen binding fragments thereof) having certain structural features. The present disclosure further relates to polynucleotides and vectors encoding such polypeptide complexes; cells, pharmaceutical compositions, and kits containing such polypeptide complexes; and methods of using such polypeptide complexes.

### BACKGROUND

Immunotherapy is the treatment of disease by activating or suppressing the immune system. In recent years, immunotherapy has become of great interest to researchers and clinicians, particularly in its promise to treat cancer and infectious disease. Therapeutic antibodies are an important type of immunotherapy. Therapeutic antibodies can be monospecific, meaning that they have specificity to one antigen or epitope. Therapeutic antibodies have also been engineered to have specificity for two different antigens or epitopes (i.e., bispecific antibodies) or for multiple different antigens or epitopes (trispecific antibodies, tetraspecific antibodies, etc.). In addition, monospecific, bispecific, and multispecific antibodies have been combined to form multi-targeting strategies to treat complex human diseases, such as cancer and infectious disease.

Cancer immunotherapy has seen tremendous progress in the past few decades. Recent developments include checkpoint inhibitors and T cell engagers. With the approval of blinatumomab in 2014, bispecific T cell engagers have become a very active field for next generation anti-cancer therapeutics. Bispecific T cell engagers are antibodies that recognize antigens expressed on target tumor cells and simultaneously engage the CD3 subunit of the T cell receptor on cytotoxic T cells, serving as an artificial immune synapse to mediate tumor cell lysis by the cytotoxic T cells.

While bispecific T cell engager antibodies show high anti-tumor potency, they are also associated with strong side effects, particularly the cytokine release syndrome (CRS) associated with on-target, off-tumor toxicity for solid tumor targets, which can limit the therapeutic potential of these antibodies. On-target, off-tumor toxicity occurs when bispecific T cell engager antibodies bind to cancer antigens expressed on cells found in normal, non-cancerous tissue in addition to cancer antigens expressed on cancerous cells, thereby recruiting cytotoxic T cells to normal tissue and causing damage to the normal tissue. Because few solid tumor antigens are exclusively specific to tumors, such toxicity poses a great challenge for cancer immunotherapy using bispecific T cell or natural killer (NK) cell engagers.

There is a need for bispecific or multispecific antigen binding polypeptide complexes that can bind specific target molecules or combinations of target molecules and activate cytotoxic T cells only when in close proximity to tumor cells or within the tumor microenvironment. There is a further need for bispecific or multispecific T cell engager antibodies that yield high efficacy with manageable adverse events (AD) and, at the same time, a wider therapeutic window and better tolerability.

### BRIEF SUMMARY

Provided herein is an antigen binding polypeptide complex comprising a first polypeptide, a second polypeptide, and a third polypeptide; wherein the first polypeptide has a structure represented by VL1-L1-VH1-L2-Fc; VH1-L1-VL1-L2-Fc; VH1-L3-VL1-L4-Fc; or VL1-L3-VH1-L4-Fc; wherein the second polypeptide has a structure represented by VH2-L5-VH3-L6-CH1-L7-Fc or VH3-L5-VH2-L6-CH1-L7-Fc; wherein the third polypeptide has a structure represented by VL2-L8-VL3-L9-CL or VL3-L8-VL2-L9-CL; wherein VL1 is a first immunoglobulin light chain variable region; VL2 is a second immunoglobulin light chain variable region; VL3 is a third immunoglobulin light chain variable region; VH1 is a first immunoglobulin heavy chain variable region; VH2 is a second immunoglobulin heavy chain variable region; VH3 is a third immunoglobulin heavy chain variable region; Fc is a region comprising an immunoglobulin heavy chain constant region 2 (CH2), an immunoglobulin heavy chain constant region 3 (CH3), and optionally, an immunoglobulin hinge; CH1 is an immunoglobulin heavy chain constant region 1; CL is an immunoglobulin light chain constant region; and L1-L9 are amino acid linkers.

Provided herein is an antigen binding polypeptide complex comprising a first polypeptide, a second polypeptide, a third polypeptide, and a fourth polypeptide; wherein the first polypeptide has a structure represented by VL1-L1-CL or VH1-L1-CL; wherein the second polypeptide has a structure represented by VH1-L2-CH1-L3-Fc or VL1-L2-CH1-L3-Fc; wherein the third polypeptide has a structure represented by VH2-L4-VH3-L5-CH1-L6-Fc or VH3-L4-VH2-L5-CH1-L6-Fc; wherein the fourth polypeptide has a structure represented by VL2-L7-VL3-L8-CL or VL3-L7-VL2-L8-CL; wherein VL1 is a first immunoglobulin light chain variable region; VL2 is a second immunoglobulin light chain variable region; VL3 is a third immunoglobulin light chain variable region; VH1 is a first immunoglobulin heavy chain variable region; VH2 is a second immunoglobulin heavy chain variable region; VH3 is a third immunoglobulin heavy chain variable region; Fc is a region comprising an immunoglobulin heavy chain constant region 2 (CH2), an immunoglobulin heavy chain constant region 3 (CH3), and optionally, an immunoglobulin hinge; CH1 is an immunoglobulin heavy chain constant region 1; CL is an immunoglobulin light chain constant region; and L1-L8 are amino acid linkers.

Provided herein is an antigen binding polypeptide complex comprising a first polypeptide, a second polypeptide, and a third polypeptide; wherein the first polypeptide has a structure represented by VL1-L1-VL2-L2-VH2-L3-VH1-L4-Fc; VL1-L1-VH2-L2-VL2-L3-VH1-L4-Fc; VH1-L5-VH2-L6-VL2-L7-VL1-L8-Fc; or VH1-L5-VL2-L6-VH2-L7-VL1-L8-Fc; wherein the second polypeptide has a structure represented by VH3-L9-VH4-L10-CH1-L11-Fc or VH4-L9-VH3-L10-CH1-L11-Fc; wherein the third polypeptide has a structure represented by VL3-L12-VL4-L13-CL or VL4-L12-VL3-L13-CL; wherein VL1 is a first immunoglobulin light chain variable region; VL2 is a second immunoglobulin light chain variable region; VL3 is a third immunoglobulin light chain variable region; VL4 is a fourth immunoglobulin light chain variable region; VH1 is a first immunoglobulin heavy chain variable region; VH2 is a second immunoglobulin heavy chain variable region; VH3 is a third immunoglobulin heavy chain variable region; VH4 is a fourth immunoglobulin heavy chain variable region; Fe is a region comprising an immunoglobulin heavy chain constant region 2 (CH2), an immunoglobulin heavy chain constant region 3 (CH3), and optionally, an immunoglobulin hinge; CH1 is an immunoglobulin heavy chain constant region 1; CL is an immunoglobulin light chain constant region; and L1-L13 are amino acid linkers.

Provided herein is an antigen binding polypeptide complex comprising a first polypeptide, a second polypeptide, and a third polypeptide; wherein the first polypeptide has a structure represented by VL1-L1-VL2-L2-VH2-L3-VH1-L4-Fc; VL1-L1-VH2-L2-VL2-L3-VH1-L4-Fc; VH1-L5-VH2-L6-VL2-L7-VL1-L8-Fc; or VH1-L5-VL2-L6-VH2-L7-VL1-L8-Fc; wherein the second polypeptide has a structure represented by VH3-L9-VH4-CH1-Fc; or VH4-L9-VH3-CH1-Fc; wherein the third polypeptide has a structure represented by VL3-L10-VL4-CL; or VL4-L10-VL3-CL; wherein VL1 is a first immunoglobulin light chain variable region; VL2 is a second immunoglobulin light chain variable region; VL3 is a third immunoglobulin light chain variable region; VL4 is a fourth immunoglobulin light chain variable region; VH1 is a first immunoglobulin heavy chain variable region; VH2 is a second immunoglobulin heavy chain variable region; VH3 is a third immunoglobulin heavy chain variable region; VH4 is a fourth immunoglobulin heavy chain variable region; Fc is a region comprising an immunoglobulin heavy chain constant region 2 (CH2), an immunoglobulin heavy chain constant region 3 (CH3), and optionally, an immunoglobulin hinge; CH1 is an immunoglobulin heavy chain constant region 1; CL is an immunoglobulin light chain constant region; and L1-L10 are amino acid linkers.

Provided herein is an antigen binding polypeptide complex comprising a first polypeptide and a second polypeptide; wherein the first polypeptide has a structure represented by VL1-L1-VL2-L2-VH2-L3-VH1-L4-Fc; VL1-L1-VH2-L2-VL2-L3-VH1-L4-Fc; VH1-L5-VH2-L6-VL2-L7-VL1-L8-Fc; or VH1-L5-VL2-L6-VH2-L7-VL1-L8-Fc; wherein the second polypeptide has a structure represented by VL3-L9-VL4-L10-VH4-L11-VH3-L12-Fc; VL3-L9-VH4-L10-VL4-L11-VH3-L12-Fc; VH3-L13-VH4-L14-VL4-L15-VL3-L16-Fc; or VH3-L13-VL4-L14-VH4-L15-VL3-L16-Fc; wherein VL1 is a first immunoglobulin light chain variable region that specifically binds to a tumor-associated antigen; VL2 is a second immunoglobulin light chain variable region that specifically binds to a tumor-associated antigen; VL3 is a third immunoglobulin light chain variable region that specifically binds to CD3; VL4 is a fourth immunoglobulin light chain variable region that specifically binds to a tumor-associated antigen; VH1 is a first immunoglobulin heavy chain variable region that specifically binds to a tumor-associated antigen; VH2 is a second immunoglobulin heavy chain variable region that specifically binds to a tumor-associated antigen; VH3 is a third immunoglobulin heavy chain variable region that specifically binds to CD3; VH4 is a fourth immunoglobulin heavy chain variable region that specifically binds to a tumor-associated antigen; Fc is a region comprising an immunoglobulin heavy chain constant region 2 (CH2), an immunoglobulin heavy chain constant region 3 (CH3), and optionally, an immunoglobulin hinge; and L1-L16 are amino acid linkers.

Provided herein is an antigen binding polypeptide complex comprising a first polypeptide and a second polypeptide; wherein the first polypeptide has a structure represented by VL1-L1-VL2-L2-VH2-L3-VH1-L4-CH1-L5-Fc; VL1-L1-VH2-L2-VL2-L3-VH1-L4-CH1-L10-Fc; VH1-L6-VH2-L7-VL2-L8-VL1-L9-CH1-L10-Fc; VH1-L6-VL2-L7-VH2-L8-VL1-L9-CH1-L10-Fc; VL1-L11-VL2-L12-VH2-L13-VH1-L14-CL-L15-Fc; VL1-L11-VH2-L12-VL2-L13-VH1-L14-CL-L15-Fc; VH1-L16-VH2-L17-VL2-L18-VL1-L19-CL-L20-Fc; VH1-L16-VL2-L17-VH2-L18-VL1-L19-CL-L20-Fc; VL1-L21-VL2-L22-VH2-L23-VH1-L24-CH1-L25-CL-L26-Fc; VL1-L21-VH2-L22-VL2-L23-VH1-L24-CH1-L25-CL-L26-Fc; VH1-L27-VH2-L28-VL2-L29-VL1-L30-CH1-L31-CL-L32-Fc; VH1-L27-VL2-L28-VH2-L29-VL1-L30-CH1-L31-CL-L32-Fc; VL1-L33-VL2-L34-VH2-L35-VH1-L36-CL-L37-CH1-L38-Fc; VL1-L33-VH2-L34-VL2-L35-VH1-L36-CL-L37-CH1-L38-Fc; VH1-L39-VH2-L40-VL2-L41-VL1-L42-CL-L43-CH1-L44-Fc; or VH1-L39-VL2-L40-VH2-L41-VL1-L42-CL-L43-CH1-L44-Fc; wherein the second polypeptide has a structure represented by VL3-L45-VL4-L46-VH4-L47-VH3-L48-CH1-L49-Fc; VL3-L45-VH4-L46-VL4-L47-VH3-L48-CH1-L49-Fc; VH3-L50-VH4-L51-VL4-L52-VL3-L53-CH1-L54-Fc; VH3-L50-VL4-L51-VH4-L52-VL3-L53-CH1-L54-Fc; VL3-L55-VL4-L56-VH4-L57-VH3-L58-CL-L59-Fc; VL3-L55-VH4-L56-VL4-L57-VH3-L58-CL-L59-Fc; VH3-L60-VH4-L61-VL4-L62-VL3-L63-CL-L64-Fc; VH3-L60-VL4-L61-VH4-L62-VL3-L63-CL-L64-Fc; VL3-L65-VL4-L66-VH4-L67-VH3-L68-CH1-L69-CL-L70-Fc; VL3-L65-VH4-L66-VL4-L67-VH3-L68-CH1-L69-CL-L70-Fc; VH3-L71-VH4-L72-VL4-L73-VL3-L74-CH1-L75-CL-L76-Fc; VH3-L71-VL4-L72-VH4-L73-VL3-L74-CH1-L75-CL-L76-Fc; VL3-L77-VL4-L78-VH4-L79-VH3-L80-CL-L81-CH1-L82-Fc; VL3-L77-VH4-L78-VL4-L79-VH3-L80-CL-L81-CH1-L82-Fc; VH3-L83-VH4-L84-VL4-L85-VL3-L86-CL-L87-CH1-L88-Fc; or VH3-L83-VL4-L84-VH4-L85-VL3-L86-CL-L87-CH1-L88-Fc; wherein VL1 is a first immunoglobulin light chain variable region that specifically binds to a tumor-associated antigen; VL2 is a second immunoglobulin light chain variable region that specifically binds to a tumor-associated antigen; VL3 is a third immunoglobulin light chain variable region that specifically binds to CD3; VL4 is a fourth immunoglobulin light chain variable region that specifically binds to a tumor-associated antigen; VH1 is a first immunoglobulin heavy chain variable region that specifically binds to a tumor-associated antigen; VH2 is a second immunoglobulin heavy chain variable region that specifically binds to a tumor-associated antigen; VH3 is a third immunoglobulin heavy chain variable region that specifically binds to CD3; VH4 is a fourth immunoglobulin heavy chain variable region that specifically binds to a tumor-associated antigen; Fc is a region comprising an immunoglobulin heavy chain constant region 2 (CH2), an immunoglobulin heavy chain constant region 3 (CH3), and optionally, an immunoglobulin hinge; CH1 is an immunoglobulin heavy chain constant region 1; CL is an immunoglobulin light chain constant region; and L1-L88 are amino acid linkers.

In some aspects, the one or more linkers L1-L88 has a length of from 0 amino acids to about 50 amino acids. In some aspects, the one or more linkers L1-L88 is non-immunogenic. In some aspects, the one or more linkers L1-L88 does not contain a consensus T cell epitope.

In some aspects, the one or more linkers L1-L88 is a cleavable linker. In some aspects, the cleavable linker is cleaved by a matrix metalloprotease (MMP), a type II transmembrane serine protease, or a MMP and a type II transmembrane serine protease. In some aspects, the MMP is MMP1, MMP2, MMP7, MMP8, MMP9, MMP13, or a combination thereof. In some aspects, the type II transmembrane serine protease is a matriptase, hepsin, or a combination thereof. In some aspects, the matriptase is matriptase 1, matriptase 2, matriptase 3, or a combination thereof. In some aspects, the cleavable linker is cleaved by urokinase or legumain.

In some aspects, the cleavable linker is GPAALV, GSGRKG, GPLGLTG, GPSGLVG, GLVGRKAG, GPAGLVG, GPAGLVSG, STRKAGG, ASTRKAG, or ASTRKAGG (SEQ ID NOs:22-31), or a sequence having at least 50%, at least 60%, at least 70%, at least 80%, at least 90%, or at least 95% identity to any one of SEQ ID NOs:22-31. In some aspects, the one or more of linkers L1-L88 comprise the amino acid sequence of g, a, gss, asg, ggssg, gssgs, gtvaa, asggs, astgg, ggsgs, asggsg, ggsgssg, ggsggssgss, sggsgssggs, ggsggsgsgggsasgsg, ggsggsgsggggsasgsg, gggssggggsggsgsggsgs, ggggsggsgsggggsasgsg, gggssggsgsggsgsggsgs, sggssggsgsggsgsggsgssg, gsgssggggsggsgsggsgssg, ggggsgsggsgggssggggsggggsggggsggggsggggs, ggggsggggsggggsggggsggggsggggsggggsggggs, ggggsgsggsgggssggggsggggsggggsggggsggggssss, or ggggsgsggsgggssggggsggggsggggsggggsggggssssgs (SEQ ID NOs:1-21) or any one of SEQ ID NOs:444, 567, 576 (GGS) and 607, or a sequence having at least 50%, at least 60%, at least 70%, at least 80%, at least 90%, or at least 95% identity to any one of SEQ ID NOs:1-21 or any one of SEQ ID NOs:444, 567, 576 (GGS) and 607.

In some aspects, one of VH1, VH2, VH3 or VH4 specifically binds to CD3. In some aspects, one of VL1, VL2, VL3 or VL4 specifically binds to CD3. In some aspects, VH1 and VL1, VH2 and VL2, VH3 and VL3, or VH4 and VL4 specifically binds to CD3.

In some aspects, the VH1, VH2, VH3 or VH4 that specifically binds to CD3 comprises a CDR1 comprising an amino acid sequence having at least 90% identity, at least 95% identity, or 100% identity to any one of SEQ ID NOs:32, 40, 96, 433, 524, 667, 703, 739, 767, 803. 839, 851 and 859; a CDR2 comprising an amino acid sequence having at least 90% identity, at least 95% identity, or 100% identity to any one of SEQ ID NOs:33, 41, 97, 434, 525, 668, 704, 740, 768, 804, 840, 852 and 860; and a CDR3 comprising an amino acid sequence having at least 90% identity, at least 95% identity, or 100% identity to any one of SEQ ID NOs:34, 42, 98, 435, 526, 669, 705, 741, 769, 805, 841, 853 and 861; and wherein the VL1, VL2, VL3 or VL4 that specifically binds to CD3 comprises a CDR1 comprising an amino acid sequence having at least 90% identity, at least 95% identity, or 100% identity to any one of SEQ ID NOs:35, 44, 99, 446, 456, 466, 476, 520, 663, 699, 735, 763, 799, 835, 855 and 863; a CDR2 comprising an amino acid sequence having at least 90% identity, at least 95% identity, or 100% identity to any one of SEQ ID NOs:36, 45, 100, 447 (DT), 457 (DT), 467 (DT), 477 (DT), 521, 664, 700, 736, 764, 800, 836, 856 and 864; and a CDR3 comprising an amino acid sequence having at least 90% identity, at least 95% identity, or 100% identity to any one of SEQ ID NOs:37, 46, 101, 448, 458, 468, 478, 522, 665, 701, 737, 765, 801, 837, 857 and 865. In some aspects, the VH1, VH2, VH3 or VH4 that specifically binds to CD3 comprises an amino acid sequence having at least 80%, at least 85%, at least 90%, at least 95%, or 100% identity to any one of SEQ ID NOs:38, 43, 102, 423, 432, 523, 666, 702, 738, 766, 802, 838, 850 and 858, and the VL1, VL2, VL3 or VL4 that specifically binds to CD3 comprises an amino acid sequence having at least 80%, at least 85%, at least 90%, at least 95%, or 100% identity to any one of SEQ ID NOs:39, 47, 103, 422, 445, 455, 465, 475, 519, 662, 698, 734, 762, 798, 834, 854 and 862.

In some aspects, one or more of VH1, VH2, VH3 or VH4 specifically binds to a tumor-associated antigen (TAA). In some aspects, one or more of VL1, VL2, VL3 or VL4 specifically binds to a TAA. In some aspects, VH1 and VL1, VH2 and VL2, VH3 and VL3, and/or VH4 and VL4 specifically binds to a TAA. In some aspects, the TAA is A2AR, APRIL, ATPDase, BAFF, BAFFR, BCMA, BlyS, BTK, BTLA, B7DC, B7H1, B7H2, B7H3, B7H4, B7H5, B7H6, B7H7, B7RP1, B7-4, C3, C5, CCL2, CCL3, CCL4, CCL5, CCL7, CCL8, CCL11, CCL15, CCL17, CCL19, CCL20, CCL21, CCL25, CCR3, CCR4, CD16A, CD19, CD20, CD24, CD27, CD28, CD30, CD38, CD39, CD40, CD40L, CD47, CD52, CD70, CD80, CD86, CD123, CD133, CD137, CD137L, CD160, CD272, CEACAM5, CLEC9, CLEC91, CRTH2, CSF-1, CSF-2, CSF-3, CXCL1, CXCL2, CXCL4, CXCL12, CXCL13, CXCR3, cMet, CTLA4, DLL3, DLL4, DNGR-1, E-cadherin, EGFR, ENTPD1, EpCAM, FCER1, FCER1A, FCER2, FGFR, FLAP, FOLH1, Gi24, GITR, GITRL, GPR5, GP100, GPRC5D, HER2, HER3, ICOSL, ICOS, HHLA2, HMGB1, HVEM, IDO, IFNa, IgE, IGF1R, IL2Rbeta, IL1, ILIA, IL1B, IL1F10, IL2, IL4, IL4Ra, IL5, IL5R, IL6, IL7, IL7Ra, IL8, IL9, IL9R, IL10, rhILlO, IL12, IL13, IL13Ra1, IL13Ra2, IL15, IL17, IL17Rb, IL18, IL22, IL23, IL25, IL7, IL33, IL35, ITGB4, ITK, KIR, LAG3, LAMP1, leptin, LPFS2, MHC class II, MUC-1, MUC-16, NCR3LG1, NKG2D, NKp46, NTPDase-1, OX40, OX40L, PD-1, PD-L1, PD-L2, PROM1, S152, SIRPalpha, SISP1, SLC, SPG64, ST2, STEAP1, STEAP2, Syk kinase, STEAP1, TROP2, TACI, TDO, TGFbeta, T14, TIGIT, TIM3, TLR, TLR2, TLR4, TLR5, TLR9, TMEF1, TNFa, TNFRSF7, Tp55, TREM1, TSLP, TSLPR, TWEAK, VEGF, VISTA, Vstm3, or WUCAM, or a combination thereof. In some aspects, the TAA is CD19. In some aspects, the TAA is CD20. In some aspects, the TAA is cMet. In some aspects, the TAA is Trop2.

In some aspects, the one or more of VH1, VH2, VH3 or VH4 that specifically binds to a TAA comprises a CDR1 comprising an amino acid sequence having at least 90% identity, at least 95% identity, or 100% identity to any one of SEQ ID NOs:64, 72, 80, 88, 104, 112, 120, 128, 389, 409, 419, 429, 659, 695, 731, 397, 508, 540, 552, 757, 793, 829, 875, 500, 749, 785, 821, 649, 685, 775, 847, 625, 641, 677 and 713; a CDR2 comprising an amino acid sequence having at least 90% identity, at least 95% identity, or 100% identity to any one of SEQ ID NOs:65, 73, 81, 89, 105, 113, 121, 129, 390, 410, 420, 430, 660, 696, 732, 398, 509, 541, 553, 758, 794, 830, 876, 501, 750, 786, 822, 650, 686, 776, 848, 626, 642, 678 and 714; and a CDR3 comprising an amino acid sequence having at least 90% identity, at least 95% identity, or 100% identity to any one of SEQ ID NOs:66, 74, 82, 90, 106, 114, 122, 130, 391, 411, 421, 431, 661, 697, 733, 399, 510, 542, 554, 759, 795, 831, 877, 502, 751, 787, 823, 651, 687, 777, 849, 627, 643, 679 and 715; and wherein the one or more of VL1, VL2, VL3 or VL4 that specifically binds to a TAA comprises a CDR1 comprising an amino acid sequence having at least 90% identity, at least 95% identity, or 100% identity to any one of SEQ ID NOs:67, 75, 83, 91, 107, 115, 123, 131, 385, 405, 415, 441, 452, 462, 472, 655, 691, 727, 393, 486, 504, 536, 548, 753, 789, 825, 871, 879, 490, 496, 745, 781, 817, 645, 681, 771, 843, 621, 637, 673 and 709; a CDR2 comprising an amino acid sequence having at least 90% identity, at least 95% identity, or 100% identity to any one of SEQ ID NOs:68, 76, 84, 92, 108, 116, 124, 132, 386 (YTS); 406 (YTS), 416 (YTS), 442 (YTS), 453 (YTS), 463 (YTS), 473 (YTS), 656, 692, 728, 394, 487 (AT), 505 (AT), 537, 549, 754, 790, 826, 872, 880, 491 (DA), 497 (DA), 746, 782, 818, 646, 682, 772, 844, 622, 638, 674 and 710; and a CDR3 comprising an amino acid sequence having at least 90% identity, at least 95% identity, or 100% identity to any one of SEQ ID NOs:69, 77, 85, 93, 109, 117, 125, 133, 387, 407, 417, 443, 454, 464, 474, 657, 693, 729, 395, 488, 506, 538, 550, 755, 791, 827, 873, 881, 492, 498, 747, 783, 819, 647, 683, 773, 845, 623, 639, 675 and 711. In some aspects, the one or more of VH1, VH2, VH3 or VH4 that specifically binds to a TAA comprises an amino acid sequence having at least 80%, at least 85%, at least 90%, at least 95%, or 100% identity to any one of SEQ ID NOs:70, 78, 86, 94, 102, 110, 118, 126, 134, 388, 408, 418, 428, 584, 658, 694, 730, 396, 403, 507, 539, 551, 756, 792, 828, 883, 866, 874, 499, 544, 556, 748, 784, 820, 528, 648, 684, 774, 846, 624, 640, 676 and 712; and the one or more of VL1, VL2, VL3 or VL4 that specifically binds to a TAA comprises an amino acid sequence having at least 80%, at least 85%, at least 90%, at least 95%, or 100% identity to any one of SEQ ID NOs:71, 79, 87, 95, 103, 111, 119, 127, 135, 384, 404, 414, 440, 451, 461, 471, 583, 654, 690, 726, 392, 402, 485, 503, 535, 547, 752, 788, 824, 870, 878, 489, 495, 543, 555, 744, 780, 816, 882, 527, 644, 680, 770, 842, 620, 636, 672 and 708.

In some aspects, one or more of VH1, VH2, VH3 or VH4 specifically binds to an immune stimulating receptor. In some aspects, one or more of VL1, VL2, VL3 or VL4 specifically binds to an immune stimulating receptor. In some aspects, VH1 and VL1, VH2 and VL2, VH3 and VL3, and/or VH4 and VL4 specifically bind to an immune stimulating receptor. In some aspects, the immune stimulating receptor is CD28.

In some aspects, the VH1, VH2, VH3 or VH4 that specifically binds to an immune stimulating receptor comprises a CDR1 comprising an amino acid sequence having at least 90% identity, at least 95% identity, or 100% identity to any one of SEQ ID NOs:48, 56, 649, 685, 775 and 847; a CDR2 comprising an amino acid sequence having at least 90% identity, at least 95% identity, or 100% identity to any one of SEQ ID NOs:49, 57, 650, 686, 776 and 848; and a CDR3 comprising an amino acid sequence having at least 90% identity, at least 95% identity, or 100% identity to any one of SEQ ID NOs:50, 58, 651, 687, 777 and 849; and wherein the VL1, VL2, VL3 or VL4 that specifically binds to an immune stimulating receptor comprises a CDR1 comprising an amino acid sequence having at least 90% identity, at least 95% identity, or 100% identity to any one of SEQ ID NOs:51, 59, 645, 681, 771 and 843; a CDR2 comprising an amino acid sequence having at least 90% identity, at least 95% identity, or 100% identity to any one of SEQ ID NOs:52, 60, 646, 682, 772 and 844; and a CDR3 comprising an amino acid sequence having at least 90% identity, at least 95% identity, or 100% identity to any one of SEQ ID NOs:53, 61, 647, 683, 773 and 845. In some aspects, the VH1, VH2, VH3 or VH4 that specifically binds to an immune stimulating receptor comprises an amino acid sequence having at least 80%, at least 85%, at least 90%, at least 95%, or 100% identity to any one of SEQ ID NOs:54, 62, 528, 648, 684, 774 and 846, and the VL1, VL2, VL3 or VL4 that specifically binds to an immune stimulating receptor comprises an amino acid sequence having at least 80%, at least 85%, at least 90%, at least 95%, or 100% identity to any one of SEQ ID NOs:55, 63, 527, 644, 680, 770 and 842.

In some aspects, L5, L8, or L5 and L8 are cleavable linkers. In some aspects, L4, L7, or L4 and L7 are cleavable linkers. In some aspects, L9, L12, or L9 and L12 are cleavable linkers. In some aspects, VH3 and VL3 specifically bind to CD3. In some aspects, VH1 and VL1 and/or VH2 and VL2 specifically bind to a TAA. In some aspects, VH1 and VL1 or VH2 and VL2 specifically bind to CD28. In some aspects, VH4 and VL4 specifically bind to CD3. In some aspects, one or more of VH1 and VL1, VH2 and VL2, and VH3 and VL3 specifically bind to a TAA. In some aspects, VH1 and VL1 specifically bind to a TAA, VH2 and VL2 specifically bind to a TAA, and VH3 and VL3 specifically bind to CD28. In some aspects, VH1 and VL1 specifically bind to CD28, VH2 and VL2 specifically bind to a TAA, and VH3 and VL3 specifically bind to a TAA. In some aspects, VH1 and VL1 specifically bind to cMet, VH2 and VL2 specifically bind to Trop2, and VH3 and VL3 specifically bind to CD28. In some aspects, VH1 and VL1 specifically bind to Trop2, VH2 and VL2 specifically bind to cMet, and VH3 and VL3 specifically bind to CD28. In some aspects, VH1 and VL1 specifically bind to cMet, VH2 and VL2 specifically bind to CD28, and VH3 and VL3 specifically bind to Trop2. In some aspects, VH1 and VL1 specifically bind to CD28, VH2 and VL2 specifically bind to cMet, and VH3 and VL3 specifically bind to Trop2. In some aspects, VH1 and VL1 specifically bind to Trop2, VH2 and VL2 specifically bind to CD28, and VH3 and VL3 specifically bind to cMet. In some aspects, VH1 and VL1 specifically bind to CD28, VH2 and VL2 specifically bind to Trop2, and VH3 and VL3 specifically bind to cMet. In some aspects, VH1 and VL1 specifically bind to CD19, VH2 and VL2 specifically bind to CD20, and VH3 and VL3 specifically bind to CD28. In some aspects, VH1 and VL1 specifically bind to CD20, VH2 and VL2 specifically bind to CD19, and VH3 and VL3 specifically bind to CD28. In some aspects, VH1 and VL1 specifically bind to CD19, VH2 and VL2 specifically bind to CD28, and VH3 and VL3 specifically bind to CD20. In some aspects, VH1 and VL1 specifically bind to CD20, VH2 and VL2 specifically bind to CD28, and VH3 and VL3 specifically bind to CD19. In some aspects, VH1 and VL1 specifically bind to CD28, VH2 and VL2 specifically bind to CD19, and VH3 and VL3 specifically bind to CD20. In some aspects, VH1 and VL1 specifically bind to CD28, VH2 and VL2 specifically bind to CD20, and VH3 and VL3 specifically bind to CD19.

In some aspects, L1, L3, or L1 and L3 are cleavable linkers, or L5, L7, or L5 and L7 are cleavable linkers. In some aspects, L9, L11, or L9 and L11 are cleavable linkers, or L13, L15, or L13 and L15 are cleavable linkers. In some aspects, VH1 and VL1, VH2 and VL2, VH3 and VL3, or VH4 and VL4 specifically bind to CD3. In some aspects, one or more of VH1 and VL1, VH2 and VL2, VH3 and VL3, and VH4 and VL4 specifically bind to a TAA. In some aspects, VH1 and VL1, VH2 and VL2, VH3 and VL3, or VH4 and VL4 specifically bind to CD28.

In some aspects, an antigen binding polypeptide complex described herein is an antibody or antigen binding fragment thereof.

In some aspects, the immunoglobulin hinge comprises an upper hinge region, a middle hinge region, a lower hinge region, or a combination thereof.

In some aspects, the Fc region comprises at least one knob-into-hole modification. In some aspects, the antigen binding polypeptide complex is an IgG1 or IgG4 antibody and the knob-into-hole modification comprises (i) knob substitutions of S354C and T366W and hole substitutions of Y349C, T366S, L368A and Y407V; (ii) hole substitutions of L234A, L235A and P329A; (iii) hole substitutions of L234A and L235A; (iv) hole substitutions of M428L and N433 S; (v) hole substitutions of M252Y, S254T and T256E; or (vi) a combination thereof, based on the EU numbering scheme.

Also provided herein is an antibody or antigen binding fragment thereof comprising an antigen binding polypeptide complex described herein.

Also provided herein is a pharmaceutical composition comprising an antigen binding polypeptide complex described herein, or an antibody or antigen binding fragment thereof, and a pharmaceutically acceptable carrier.

Provided herein is a kit comprising an antigen binding polypeptide complex, an antibody or antigen binding fragment thereof, or a pharmaceutical composition described herein, and instructions for use.

Also provided herein are specified methods of use of an antigen binding polypeptide complex, antibody or antigen binding fragment thereof, pharmaceutical composition or kit described herein, or a combination thereof. Provided herein is a method for treating cancer comprising administering to a subject in need thereof an antigen binding polypeptide complex, an antibody or antigen binding fragment thereof, or a pharmaceutical composition described herein. In some aspects, the cancer is a solid cancer. In some aspects, the cancer is breast cancer, lung cancer, gastric cancer, prostate cancer, cervical cancer, urothelial cancer, or pancreatic cancer. In some aspects, the cancer is a hematological cancer. In some aspects, the hematological cancer is leukemia or lymphoma. In some aspects, the leukemia or lymphoma is B cell leukemia, B cell lymphoma, diffuse large B-cell lymphoma (DLBCL) Follicular lymphoma, Chronic lymphocytic leukemia (CLL)/small lymphocytic lymphoma (SLL), Mantle cell lymphoma (MCL), Burkitt lymphoma, Lymphoplasmacytic lymphoma (Waldenstrom macroglobulinemia), prolymphocytic leukemia (PLL), or hairy cell leukemia (HCL).

### BRIEF DESCRIPTION OF THE DRAWINGS

FIG. 1A depicts configurations of masked trispecific T cell engagers. Fv1-Fv2 are against tumor associated antigens (TAAs) or immune costimulatory receptors. The third Fv targets CD3.

FIG. 1B depicts a configuration of masked tetraspecific T cell engagers. Fv1-Fv3 are against tumor associated antigens (TAAs) or immune costimulatory receptors. The fourth Fv targets CD3.

FIG. 2 shows SDS-PAGE results of in vitro cleavage of exemplary masked tetraspecific molecules as depicted. Molecules were treated with either MTP or MMP9 protease as specified.

FIG. 3 shows ELISA binding results of exemplary masked tetraspecific molecules as depicted in FIG. 2, or negative isotype (Control IgG1), with or without protease treatment. Molecules cleaved or not cleaved by MTP or MMP9 as specified were tested for binding affinity to Trop2 and cMet.

FIG. 4 shows ELISA binding results of exemplary masked tetraspecific molecules as depicted in FIG. 2, or negative isotype (Control IgG1), with or without protease treatment. Molecules cleaved or not cleaved by MTP or MMP9 as specified were tested for binding affinity to CD28.

FIG. 5 shows ELISA binding results of exemplary masked tetraspecific molecules as depicted in FIG. 2, or negative isotype (Control IgG1), with or without protease treatment. Molecules cleaved or not cleaved by MTP or MMP9 as specified were tested for binding affinity to CD3.

FIG. 6 shows cytolysis of HCC1954 tumor cells by peripheral blood mononuclear cells PBMCs) (E:T:10:1) mediated by exemplary masked tetraspecific molecules as depicted in FIG. 2, or negative isotype (Control IgG1), from PBMCs of two donors (KP63250 and KP63251).

FIG. 7 shows ELISA binding results of exemplary non-masked tetraspecific molecules as depicted, or negative isotype (hIgG1LALPA) control, to their respective targets of hTrop2, hcMet, hCD28, and hCD3.

FIG. 8 shows CD69+ activation by exemplary non-masked tetraspecific molecules, or negative isotype (IgG1LALPA) control, of CD2+ T cells from PBMCs of two different donors.

FIG. 9 shows ELISA binding results (OD650) of MX612 and MX613 to their targets hTrop2, hcMet, hCD28, and hCD3. Results from a control antibody (control IgG), which does not bind these targets, is also shown.

FIG. 10 shows simultaneous co-binding of MX612 to its targets hTrop2, hcMet, hCD28, and hCD3, measured by biolayer-interferometry.

FIG. 11 shows simultaneous co-binding of MX446 to its targets hTrop2, hcMet, hCD28, and hCD3, measured by biolayer-interferometry.

FIG. 12 shows the ability of exemplary non-masked tetraspecific molecules to activate CD4+ and CD8+ T cells (CD69+). Results from PBMCs from six different donors in the absence of target tumor cells and following treatment with 1 nM MX446 and MX612 are shown. Results from a negative control (isotype IgG1LALPA; “−Ctl”) and positive control (CD3+CD28 mAbs; “+Ctl”) are also shown.

FIG. 13A-13B shows the ability of exemplary non-masked tetraspecific molecules to initiate release of the cytokines IL-2, IFN-gamma, IL-6 and TNFalpha. Results from PBMCs from six different donors in the absence of target tumor cells and following treatment with 1 nM MX446 and MX612 are shown. Results from a negative control (isotype IgG1LALPA; “−Ctl”) and positive control (CD3+CD28 mAbs; “+Ctl”) are also shown.

FIG. 14A-14B shows the ability of exemplary non-masked tetraspecific molecules to initiate release of the cytokines IL-2, IFN-gamma, IL-6 and TNFalpha. Results from PBMCs from six different donors in the presence of HCC1954 tumor cells and following treatment with 62.5 pM MX446 and MX612 are shown. Results from a negative control (isotype IgG1LALPA; “−Ctl”) are also shown.

FIG. 15A-15B shows the ability of exemplary non-masked tetraspecific molecules to initiate release of the cytokines IL-2, IFN-gamma, IL-6 and TNFalpha. Results from PBMCs from six different donors in the presence of BT20 tumor cells and following treatment with 62.5 pM MX446 and MX612 are shown. Results from a negative control (isotype IgG1LALPA; “−Ctl”) are also shown.

FIG. 16A-16B shows cytolysis of tumor cell lines by exemplary non-masked tetraspecific molecules or a negative control from PBMCs (E:T 5:1). HCC1954, BT-20, HCC1143 and HCC70 breast cancer cells, PC3 and DU145 prostate cancer cells, and Hs746T and N87 gastric cancer cells were used. Percent lysis of cells following treatment with MX446 and MX612, and EC50 values in pM are shown. Results from a negative control (isotype IgG1LALAPA) are also shown.

FIG. 17 summarizes cytolysis potencies of various tumor cell lines mediated by exemplary non-masked tetraspecific molecules.

FIG. 18A-18C shows binding of exemplary non-masked tetraspecific molecules to their targets hTrop2, hcMet, hCD28, and hCD3, measured by biolayer-interferometry. Results are shown for cells treated with MX974 (FIG. 18A), MX975 (FIG. 18B) and MX976 (FIG. 18C).

FIG. 19 shows in vitro cytolysis of HCC1954 tumor cells by exemplary non-masked tetraspecific molecules. Cytolysis mediated by MX974, MX975 and MX976 or a negative control from PBMCs (E:T: 5:1) was measured from a representative donor after 48 and 72 hours of incubation at 37° C. Results are shown as percent lysis of cells with increasing concentration of antibody. Results from a negative control (hIgG1LALAPA) are also shown.

FIG. 20 shows activation of CD4+ and CD8+ T cells (CD69+) from PBMCs by exemplary masked tetraspecific molecules. Results are shown as the percentage of CD69+ cells from six donors in the absence of target tumor cells, following treatment with MX444 or MX634 at 1 nM. Results from a negative control (isotype IgG1LALPA; “−Ctl”) and a positive control (CD3+CD28 mAbs; “+Ctl”) are also shown.

FIG. 21A-21B shows the ability of exemplary non-masked tetraspecific molecules to initiate release of the cytokines IL-2, IFN-gamma, IL-6 and TNFalpha. Results from PBMCs from six different donors in the absence of target tumor cells and following treatment with 1 nM MX444 and MX634 are shown. Results from a negative control (isotype IgG1LALPA; “−Ctl”) and positive control (CD3+CD28 mAbs; “+Ctl”) are also shown.

FIG. 22A-22B shows the ability of exemplary non-masked tetraspecific molecules to initiate release of the cytokines IL-2, IFN-gamma, IL-6 and TNFalpha. Results from PBMCs from six different donors in the absence of target tumor cells and following treatment with 1 nM MX444 and MX634 are shown. Results from a negative control (isotype IgG1LALPA; “−Ctl”) and positive control (CD3+CD28 mAbs; “+Ctl”) are also shown.

FIG. 23A-23B shows the ability of exemplary non-masked tetraspecific molecules to initiate release of the cytokines IL-2, IFN-gamma, IL-6 and TNFalpha. Results from PBMCs from six different donors in the presence of HCC1954 tumor cells and following treatment with 62.5 pM MX444 and MX634 are shown. Results from a negative control (isotype IgG1LALPA; “−Ctl”) are also shown.

FIG. 24A-24B shows the ability of exemplary non-masked tetraspecific molecules to initiate release of the cytokines IL-2, IFN-gamma, IL-6 and TNFalpha. Results from PBMCs from six different donors in the presence of BT20 tumor cells and following treatment with 62.5 pM MX444 and MX634 are shown. Results from a negative control (isotype IgG1LALPA; “−Ctl”) are also shown.

FIG. 25 summarizes cytolysis potencies of various tumor cell lines mediated by exemplary non-masked tetraspecific molecules.

FIG. 26 shows the structures of MX433, MX434, MX435 and MX436.

FIG. 27 shows the ability of exemplary non-masked tetraspecific molecules MX433, MX434, MX435 and MX436 to bind to their targets, as tested by ELISA. Binding results to hCD19, hCD28 and hCD3 are shown. Results from a negative control (hIgG1LALPA) are also shown.

FIG. 28 shows the structures of MX647, MX648, MX685 and MX686.

FIG. 29 shows the ability of exemplary non-masked tetraspecific molecules MX647, MX648, MX685 and MX686 to bind to their targets, as tested by ELISA. Binding results to hCD19, hCD28 and hCD3 are shown.

FIG. 30 shows surface staining of exemplary non-masked tetraspecific molecules to hCD20-expressing Epi293 cells. The molecules tested were MX647, MX789, MX788, MX787, MX786, MX685, MX793, MX792, MX791 and MX790. Staining with a negative control (hIgG1LALPA) is also shown.

FIG. 31 shows the structures of MX647, MX648, MX685, MX786 and MX790.

FIG. 32 shows ability of exemplary non-masked tetraspecific molecules to activate T cells (CD69+). The molecules tested were MX647, MX648, MX685, MX786 and MX790. The percentage of CD69+ cells from PBMCs of a representative donor in the absence of tumor cells and following treatment with MX647, MX648, MX685, MX786 and MX790 are shown. EC50 values in pM and results from a negative control (hIgG1LALAPA) are also shown.

FIG. 33 shows the measurement of cytolysis mediated by MX647, MX648, MX685, MX786 and MX790 or a negative control (hIgG1LALAPA) from PBMCs (E:T: 5:1). Results are shown as percent lysis with increasing concentration of antibody. EC50 in pM and Rmax values are also shown.

FIG. 34 shows cytokine release from PBMCs from a representative donor in the absence of target tumor cells and following treatment with 8 pM or 40 pM MX647, MX648, MX685, MX786 and MX790. Results from a negative control (hIgG1LALPA) are also shown.

FIG. 35 shows cytokine release from PBMCs from a representative donor in the presence of Z-138 tumor cells and following treatment with 8 pM or 40 pM MX647, MX648, MX685, MX786 and MX790. Results from a negative control (hIgG1LALAPA) are also shown.

FIG. 36 shows surface staining of exemplary trispecific molecules MX848, MX856 and MX857 to hCD20-expressing Epi293 cells. Staining with a negative control (control) is also shown.

FIG. 37 shows in vitro cytolysis of Z-138 tumor cells mediated by MX857 or a negative control (hIgG1LALAPA) from PBMCs from two representative donors (E:T: 3:1). Results are shown as percent lysis of cells with increasing concentration of antibody.

FIG. 38 shows surface staining of 12.5 nM MX846, MX854 and MX855 to hCD20-expressing Epi293 cells. Staining with a negative control (control) is also shown.

FIG. 39A-39B shows surface staining of 12.5 nM MX850, MX851, MX852 and MX853 to hCD20-expressing Epi293 cells. Staining with a negative control (control) is also shown.

FIG. 40 shows in vitro cytolysis of Z-138 tumor cells mediated by MX855. Results are shown as percent lysis of cells with increasing concentration of antibody.

FIG. 41A-41B show binding of MX977 and MX978 to their targets CD20, CD19, CD28 and CD3 by biolayer-interferometry.

FIG. 42 shows surface staining of 12.5 nM MX977 and MX978 to hCD20-expressing Epi293 cells. Staining with a negative control (hIgGLALAPA) is also shown.

FIG. 43 shows in vitro cytolysis of Toledo tumor cells mediated by MX977, MX978 or a negative control (hIgG1LALAPA) from PBMCs from a representative donor (E:T: 5:1). Results are shown as percent lysis of cells with increasing concentration of antibody.

### DETAILED DESCRIPTION OF THE INVENTION

The invention is directed to antigen binding polypeptide complexes (e.g., antibodies or antigen binding fragments thereof) having improved features. In some aspects, the invention enables the generation of multispecific and multifunctional antigen binding polypeptide complexes through the expression of complementary self-assembling heavy and light chains expressed with a single polypeptide per arm and, optionally, with the addition of specific amino acid linkers. Because of this multifunctionality, antigen binding polypeptide complexes of the invention can bind to specific combinations of target molecules for selectivity or breadth/neutralization, bring together two or more cell types, bring together targets and deliver activation signals, modify the disease microenvironment, and enhance avidity of binding for improved potency.

Various terms relating to aspects of disclosure are used throughout the specification and claims. Such terms are to be given their ordinary meaning in the art, unless otherwise indicated. Other specifically defined terms are to be construed in a manner consistent with the definition provided herein.

### Definitions

As used herein, the term “antigen binding polypeptide complex” refers to a group of two, three, four, or more associated polypeptides, wherein at least one polypeptide has the ability to specifically bind to one or more antigens. An antigen binding polypeptide complex, includes, but is not limited to, an antibody or antigen binding fragment thereof.

The term “antibody” includes, without limitation, a glycoprotein immunoglobulin which binds specifically to an antigen and comprises at least two heavy (H) chains and two light (L) chains interconnected by disulfide bonds. Each H chain comprises a heavy chain variable region (abbreviated herein as VH) and a heavy chain constant region. The heavy chain constant region comprises three constant domains, CH1, CH2 and CH3. Each L chain comprises a light chain variable region (abbreviated herein as VL) and a light chain constant region. The light chain constant region comprises one constant domain, CL. The VH and VL regions can be further subdivided into regions of hypervariability, termed complementarity determining regions (CDRs), interspersed with regions that are more conserved, termed framework regions (FR). Each VH and VL comprises three CDRs and four FRs, arranged from amino-terminus to carboxy-terminus in the following order: FR1, CDR1, FR2, CDR2, FR3, CDR3, FR4. The variable regions of the heavy and light chains contain a binding domain that interacts with an antigen. The constant regions of the antibodies may mediate the binding of the immunoglobulin to host tissues or factors, including various cells of the immune system (e.g., effector cells) and the first component (C1q) of the classical complement system. A heavy chain may have the C-terminal lysine or not. Unless specified otherwise herein, the amino acids in the variable regions are numbered using the Kabat numbering system and those in the constant regions are numbered using the EU system.

The term “monoclonal antibody,” as used herein, refers to an antibody that is produced by a single clone of B-cells and binds to the same epitope. In contrast, the term “polyclonal antibody” refers to a population of antibodies that are produced by different B-cells and bind to different epitopes of the same antigen. The term “antibody” includes, by way of example, monoclonal and polyclonal antibodies; chimeric and humanized antibodies; human or non-human antibodies; wholly synthetic antibodies; and single chain antibodies. A non-human antibody can be humanized by recombinant methods to reduce its immunogenicity in man.

The antibody can be an antibody that has been altered (e.g., by mutation, deletion, substitution, conjugation to a non-antibody moiety). For example, an antibody can include one or more variant amino acids (compared to a naturally occurring antibody) which change a property (e.g., a functional property) of the antibody. For example, several such alterations are known in the art, which affect, e.g., half-life, effector function, and/or immune responses to the antibody in a patient. The term antibody also includes artificial polypeptide constructs, which comprise at least one antibody-derived antigen binding site.

An “antigen binding fragment” refers to one or more fragments or portions of an antibody that retain the ability to bind specifically to the antigen bound by the whole antibody. It has been shown that the antigen binding function of an antibody can be performed by fragments or portions of a full-length antibody. An antigen binding fragment can contain the antigenic determining regions of an intact antibody (e.g., the complementarity determining regions (CDRs)). Examples of antigen binding fragments of antibodies include, but are not limited to, Fab, Fab′, F(ab′)₂, and Fv fragments, linear antibodies, and single chain antibodies. An antigen binding fragment of an antibody can be derived from any animal species, such as rodents (e.g., mouse, rat, or hamster) and humans or can be artificially produced.

Furthermore, although the two domains of the Fv fragment, VL and VH, are coded for by separate genes, they can be joined, using recombinant methods, by a synthetic linker that enables them to be made as a single protein chain in which the VL and VH regions pair to form monovalent molecules (known as single chain Fv (scFv); see, e.g., Bird et al. (1988) Science 242:423-426; and Huston et al. (1988) Proc. Natl. Acad. Sci. USA 85:5879-5883). Such single chain antibodies are also intended to be encompassed within the term “antigen-binding fragment” of an antibody.

Antigen binding fragments are obtained using conventional techniques known to those with skill in the art, and the fragments are screened for utility in the same manner as are intact antibodies. Antigen binding fragments can be produced by recombinant DNA techniques, or by enzymatic or chemical cleavage of intact immunoglobulins.

As used herein, the term “variable region” typically refers to a portion of an antibody, generally, a portion of a light or heavy chain, typically about the amino-terminal 110 to 120 amino acids, or 110 to 125 amino acids in the mature heavy chain and about 90 to 115 amino acids in the mature light chain, which differ extensively in sequence among antibodies and are used in the binding and specificity of a particular antibody for its particular antigen. The variability in sequence is concentrated in those regions called complementarity determining regions (CDRs) while the more highly conserved regions in the variable domain are called framework regions (FR). Without wishing to be bound by any particular mechanism or theory, it is believed that the CDRs of the light and heavy chains are primarily responsible for the interaction and specificity of an antibody with antigen. In some aspects, the variable region is a mammalian variable region, e.g., a human, mouse or rabbit variable region. In some aspects, the variable region comprises rodent or murine CDRs and human framework regions (FRs). In some aspects, the variable region is a primate (e.g., non-human primate) variable region. In some aspects, the variable region comprises rodent or murine CDRs and primate (e.g., non-human primate) framework regions (FRs).

The terms “complementarity determining region” or “CDR”, as used herein, refer to each of the regions of an antibody variable domain which are hypervariable in sequence and/or form structurally defined loops (hypervariable loops) and/or contain the antigen-contacting residues. Antibodies can comprise six CDRs, e.g., three in the VH and three in the VL.

The terms “VL”, “VL region,” and “VL domain” are used herein interchangeably to refer to the light chain variable region of an antigen binding polypeptide complex, antibody or antigen binding fragment thereof. In some aspects, a VL region is referred to herein as VL1 to denote a first light chain variable region, VL2 to denote a second light chain variable region, VL3 to denote a third light chain variable region, and so on. An enumerated VL region (e.g., VL1) can have the same or different antigen binding properties and/or the same or different sequence as another enumerated VL region (e.g., VL2).

The terms “VH”, “VH region,” and “VH domain” are used herein interchangeably to refer to the heavy chain variable region of an antigen binding polypeptide complex, antibody or antigen binding fragment thereof. In some aspects, a VH region is referred to herein as VH1 to denote a first heavy chain variable region, VH2 to denote a second heavy chain variable region, VH3 to denote a third heavy chain variable region, and so on. An enumerated VH region (e.g., VH1) can have the same or different antigen binding properties and/or the same or different sequence as another enumerated VH region (e.g., V12).

| As used herein, “Kabat numbering” and like terms are recognized in the art and refer to a system of numbering amino acid residues in the heavy and light chain variable regions of an antibody or antigen binding fragment thereof. In some aspects, CDRs can be determined according to the Kabat numbering system (see, e.g., Kabat E A & Wu T T (1971) Ann NY Acad Sci 190: 382-391 and Kabat E A et al., (1991) Sequences of Proteins of Immunological Interest, Fifth Edition, U.S. Department of Health and Human Services, NIH Publication No. 91-3242). Using the Kabat numbering system, CDRs within an antibody heavy chain molecule are typically present at amino acid positions 31 to 35, which optionally can include one or two additional amino acids, following 35 (referred to in the Kabat numbering scheme as 35A and 35B) (CDR1), amino acid positions 50 to 65 (CDR2), and amino acid positions 95 to 102 (CDR3). Using the Kabat numbering system, CDRs within an antibody light chain molecule are typically present at amino acid positions 24 to 34 (CDR1), amino acid positions 50 to 56 (CDR2), and amino acid positions 89 to 97 (CDR3).

As used herein, the terms “constant region” or “constant domain” are used interchangeably to refer to a portion of an antigen binding polypeptide complex, antibody or antigen binding fragment thereof, e.g., a carboxyl terminal portion of a light and/or heavy chain which is not directly involved in binding of an antibody to antigen but which can exhibit various effector functions, such as interaction with the Fc region. The constant region generally has a more conserved amino acid sequence relative to a variable region. In some aspects, an antigen binding polypeptide complex, antibody or antigen binding fragment thereof comprises a constant region or portion thereof that is sufficient for antibody-dependent cell-mediated cytotoxicity (ADCC), antibody-dependent cellular phagocytosis (ADCP), and complement-dependent cytotoxicity (CDC).

As used herein, the terms “fragment crystallizable region,” “Fc region,” or “Fc domain” are used interchangeably herein to refer to the tail region of an antibody that interacts with cell surface receptors called Fc receptors and some proteins of the complement system. Fc regions typically comprise CH2 and CH3 regions, and, optionally, an immunoglobulin hinge.

As used herein, the terms “immunoglobulin hinge,” “hinge,” “hinge domain” or “hinge region” are used interchangeably to refer to a stretch of heavy chains between the Fab and Fc portions of an antigen binding polypeptide complex, antibody or antigen binding fragment thereof. A hinge provides structure, position and flexibility, which assist with normal functioning of antibodies (e.g., for crosslinking two antigens or binding two antigenic determinants on the same antigen molecule). An immunoglobulin hinge is divided into upper, middle and lower hinge regions that can be separated based on structural and/or genetic components. An immunoglobulin hinge of the invention can contain one, two or all three of these regions. Structurally, the upper hinge region stretches from the C terminal end of CH1 to the first hinge disulfide bond. The middle hinge region stretches from the first cysteine to the last cysteine in the hinge. The lower hinge region extends from the last cysteine to the glycine of CH2. The cysteines present in the hinge form interchain disulfide bonds that link the immunoglobulin monomers.

As used herein, the term “Fab” refers to a region of an antibody that binds to an antigen. It is typically composed of one constant and one variable domain of each of the heavy and the light chain.

As used herein, the term “heavy chain” refers to a portion of an antigen binding polypeptide complex, antibody or antigen binding fragment thereof typically composed of a heavy chain variable region (VH), a heavy chain constant region 1 (CH1), a heavy chain constant region 2 (CH2), and a heavy chain constant region 3 (CH3). A typical antibody is composed of two heavy chains and two light chains. When used in reference to an antibody, a heavy chain can refer to any distinct type, e.g., alpha (α), delta (δ), epsilon (ε), gamma (γ), and mu (μ), based on the amino acid sequence of the constant region, which gives rise to IgA, IgD, IgE, IgG, and IgM classes of antibodies, respectively, including subclasses of IgG, e.g., IgG1, IgG2, IgG3, and IgG4. Heavy chain amino acid sequences are known in the art. In some aspects, the heavy chain is a human heavy chain.

As used herein, the term “light chain” refers to a portion of an antigen binding polypeptide complex, antibody or antigen binding fragment thereof typically composed of a light chain variable region (VL) and a light chain constant region (CL). A typical antibody is composed of two light chains and two heavy chains. When used in reference to an antibody, a light chain can refer to any distinct type, e.g., kappa (κ) or lambda (λ), based on the amino acid sequence of the constant region. Light chain amino acid sequences are known in the art. In some aspects, the light chain is a human light chain.

The term “chimeric” antibody or antigen binding fragment thereof refers to an antibody or antigen binding fragments thereof wherein the amino acid sequence is derived from two or more species. Typically, the variable region of both light and heavy chains corresponds to the variable region of antibodies or antigen binding fragments thereof derived from one species of mammals (e.g., mouse, rat, rabbit, etc.) with the desired specificity, affinity and capability, while the constant regions are homologous to the sequences in antibodies or antigen binding fragments thereof derived from another (usually human) to avoid eliciting an immune response in that species.

The term “humanized” antibody or antigen binding fragment thereof refers to forms of non-human (e.g., murine) antibodies or antigen binding fragments that are specific immunoglobulin chains, chimeric immunoglobulins, or fragments thereof that contain minimal non-human (e.g., murine) sequences. Typically, humanized antibodies or antigen binding fragments thereof are human immunoglobulins in which residues from a complementary determining region (CDR) are replaced by residues from a CDR of a non-human species (e.g., mouse, rat, rabbit, hamster) that have the desired specificity, affinity, and capability (Jones et al., Nature 321:522-525 (1986); Riechmann et al., Nature 332:323-327 (1988); Verhoeyen et al., Science 239:1534-1536 (1988)). In some aspects, the Fv framework region (FR) residues of a human immunoglobulin are replaced with the corresponding residues in an antibody or fragment from a non-human species that has the desired specificity, affinity, and capability. The humanized antibody or antigen binding fragment thereof can be further modified by the substitution of additional residues either in the Fv framework region and/or within the replaced non-human residues to refine and optimize antibody or antigen-binding fragment thereof specificity, affinity, and/or capability. In general, a humanized antibody or antigen binding fragment thereof will comprise substantially all of at least one, and typically two or three, variable domains containing all or substantially all of the CDR regions that correspond to the non-human immunoglobulin whereas all or substantially all of the FR regions are those of a human immunoglobulin consensus sequence. A humanized antibody or antigen binding fragment thereof can also comprise at least a portion of a constant region, typically that of a human immunoglobulin. Examples of methods used to generate humanized antibodies are known and described, for example, in U.S. Pat. No. 5,225,539; Roguska et al., Proc. Natl. Acad. Sci., USA, 91(3):969-973 (1994), and Roguska et al., Protein Eng. 9(10):895-904 (1996).

The term “human” antibody or antigen binding fragment thereof, as used herein, means an antibody or antigen binding fragment thereof having an amino acid sequence derived from a human immunoglobulin gene locus, where such antibody or antigen binding fragment is made using recombinant techniques known in the art. This definition of a human antibody or antigen binding fragment thereof includes intact or full-length antibodies and fragments thereof.

A polypeptide complex, antibody, antigen binding fragment thereof, polynucleotide, vector, or cell which is “isolated” is a polypeptide complex, antibody, antigen binding fragment thereof, polynucleotide, vector, or cell which is in a form not found in nature. Isolated polypeptide complexes, antibodies, antigen binding fragments thereof, polynucleotides, vectors, or cells include those which have been purified to a degree that they are no longer in a form in which they are found in nature. In some aspects, a polypeptide complex, antibody, antigen binding fragment thereof, polynucleotide, vector, or cell which is isolated is substantially pure. As used herein, “substantially pure” refers to material which is at least 50% pure (i.e., free from contaminants), at least 90% pure, at least 95% pure, at least 98% pure, or at least 99% pure.

The terms “polypeptide,” “peptide,” and “protein” are used interchangeably herein to refer to polymers of amino acids of any length. The polymer can be linear or branched, it can comprise modified amino acids, and it can be interrupted by non-amino acids. The terms also encompass an amino acid polymer that has been modified naturally or by intervention; for example, disulfide bond formation, glycosylation, lipidation, acetylation, phosphorylation, or any other manipulation or modification, such as conjugation with a labeling component. Also included within the definition are, for example, polypeptides containing one or more analogs of an amino acid (including, for example, unnatural amino acids, etc.), as well as other modifications known in the art. It is understood that, because the polypeptides of this invention are based upon antibodies, in some aspects, the polypeptides can occur as single chains or associated chains.

| The use of the alternative (e.g., “or”) should be understood to mean either one, both, or any combination thereof of the alternatives. As used herein, the indefinite articles “a” or “an” should be understood to refer to “one or more” of any recited or enumerated component.

As used herein, the term “and/or” is to be taken as specific disclosure of each of the two specified features or components with or without the other. Thus, the term “and/or” as used in a phrase such as “A and/or B” herein is intended to include “A and B,” “A or B,” “A” (alone), and “B” (alone). Likewise, the term “and/or” as used in a phrase such as “A, B, and/or C” is intended to encompass each of the following aspects: A, B, and C; A, B, or C; A or C; A or B; B or C; A and C; A and B; B and C; A (alone); B (alone); and C (alone).

It is understood that wherever aspects are described herein with the language “comprising,” “having” and the like, otherwise analogous aspects described in terms of “consisting of” and/or “consisting essentially of” are also provided.

As used herein, the term “about” refers to a value or composition that is within an acceptable error range for the particular value or composition as determined by one of ordinary skill in the art, which will depend in part on how the value or composition is measured or determined, i.e., the limitations of the measurement system. For example, “about” can mean within 1 or more than 1 standard deviation per the practice in the art. Alternatively, “about” can mean a range of up to 10% or 20% (i.e., ±10% or 20%). For example, about 3 mg can include any number between 2.7 mg and 3.3 mg (for 10%) or between 2.4 mg and 3.6 mg (for 20%). Furthermore, particularly with respect to biological systems or processes, the terms can mean up to an order of magnitude or up to 5-fold of a value. When particular values or compositions are provided in the application and claims, unless otherwise stated, the meaning of “about” should be assumed to be within an acceptable error range for that particular value or composition.

As described herein, any numerical range, concentration range, percentage range, ratio range or integer range is to be understood to include the value of any integer within the recited range and, when appropriate, fractions thereof (such as one-tenth and one-hundredth of an integer), unless otherwise indicated.

Unless defined otherwise, all technical and scientific terms used herein have the same meaning as commonly understood by one of ordinary skill in the art to which this disclosure is related. For example, the Concise Dictionary of Biomedicine and Molecular Biology, Juo, Pei-Show, 2nd ed., 2002, CRC Press; The Dictionary of Cell and Molecular Biology, 5th ed., 2013, Academic Press; and the Oxford Dictionary Of Biochemistry And Molecular Biology, 2006, Oxford University Press, provide one of skill with a general dictionary of many of the terms used in this disclosure.

Units, prefixes, and symbols are denoted in their Système International de Unites (SI) accepted form. Numeric ranges are inclusive of the numbers defining the range. The headings provided herein are not limitations of the various aspects of the disclosure, which can be had by reference to the specification as a whole. Accordingly, the terms defined herein are more fully defined by reference to the specification in its entirety.

Various aspects are described in further detail in the following sections.

### Antigen Binding Polypeptide Complexes

In some aspects, the invention is directed to antigen binding polypeptide complexes having certain structural features described further herein. In some aspects, an antigen binding polypeptide complex of the invention (e.g., antibody or antigen binding fragment thereof) comprises one or more cleavable linkers, as described further herein. In some aspects, an antigen binding polypeptide complex of the invention (e.g., antibody or antigen binding fragment thereof) comprises an interferon alpha, as described further herein.

In some aspects, an antigen binding polypeptide complex of the invention (e.g., antibody or antigen binding fragment thereof) contains a VH and/or VL that specifically binds to CD3. In some aspects, one of VH1, VH2, VH3 or VH4 of an antigen binding polypeptide described herein specifically binds to CD3. In some aspects, one of VL1, VL2, VL3 or VL4 of an antigen binding polypeptide described herein specifically binds to CD3. In some aspects, VH1 and VL1, VH2 and VL2, VH3 and VL3, or VH4 and VL4 of an antigen binding polypeptide described herein specifically binds to CD3.

In some aspects, the VH and/or VL that specifically binds to CD3 is masked by the interferon alpha and/or by another VH and/or VL that is joined to the VH and/or VL that specifically binds to CD3 by a cleavable linker.

In some aspects, VH1 and VH2 of an antigen binding polypeptide complex described herein are joined by a cleavable linker. In some aspects, VH1 and VL2 of an antigen binding polypeptide complex described herein are joined by a cleavable linker. In some aspects, VL1 and VH2 of an antigen binding polypeptide complex described herein are joined by a cleavable linker. In some aspects, VL1 and VL2 of an antigen binding polypeptide complex described herein are joined by a cleavable linker. In some aspects, VH1 and VH2 of an antigen binding polypeptide complex described herein are joined by a noncleavable linker having the amino acid sequence of GS (a GS linker). In some aspects, VH1 and VL2 of an antigen binding polypeptide complex described herein are joined by a GS linker. In some aspects, VL1 and VH2 of an antigen binding polypeptide complex described herein are joined by a GS linker. In some aspects, VL1 and VL2 of an antigen binding polypeptide complex described herein are joined by a GS linker.

In some aspects, VL2 and VL1 of an antigen binding polypeptide complex described herein are joined by a cleavable linker. In some aspects, VH2 and VL1 of an antigen binding polypeptide complex described herein are joined by a cleavable linker. In some aspects, VL2 and VH1 of an antigen binding polypeptide complex described herein are joined by a cleavable linker. In some aspects, VH2 and VH1 of an antigen binding polypeptide complex described herein are joined by a cleavable linker. In some aspects, VL2 and VL1 of an antigen binding polypeptide complex described herein are joined by a GS linker. In some aspects, VH2 and VL1 of an antigen binding polypeptide complex described herein are joined by a GS linker. In some aspects, VL2 and VH1 of an antigen binding polypeptide complex described herein are joined by a GS linker. In some aspects, VH2 and VH1 of an antigen binding polypeptide complex described herein are joined by a GS linker.

In some aspects, VH3 and VH4 of an antigen binding polypeptide complex described herein are joined by a cleavable linker. In some aspects, VH3 and VL4 of an antigen binding polypeptide complex described herein are joined by a cleavable linker. In some aspects, VL3 and VH4 of an antigen binding polypeptide complex described herein are joined by a cleavable linker. In some aspects, VL3 and VL4 of an antigen binding polypeptide complex described herein are joined by a cleavable linker. In some aspects, VH3 and VH4 of an antigen binding polypeptide complex described herein are joined by a GS linker. In some aspects, VH3 and VL4 of an antigen binding polypeptide complex described herein are joined by a GS linker. In some aspects, VL3 and VH4 of an antigen binding polypeptide complex described herein are joined by a GS linker. In some aspects, VL3 and VL4 of an antigen binding polypeptide complex described herein are joined by a GS linker.

In some aspects, VL4 and VL3 of an antigen binding polypeptide complex described herein are joined by a cleavable linker. In some aspects, VH4 and VL3 of an antigen binding polypeptide complex described herein are joined by a cleavable linker. In some aspects, VL4 and VH3 of an antigen binding polypeptide complex described herein are joined by a cleavable linker. In some aspects, VH4 and VH3 of an antigen binding polypeptide complex described herein are joined by a cleavable linker. In some aspects, VL4 and VL3 of an antigen binding polypeptide complex described herein are joined by a GS linker. In some aspects, VH4 and VL3 of an antigen binding polypeptide complex described herein are joined by a GS linker. In some aspects, VL4 and VH3 of an antigen binding polypeptide complex described herein are joined by a GS linker. In some aspects, VH4 and VH3 of an antigen binding polypeptide complex described herein are joined by a GS linker.

In some aspects, VH1 and VH2 of an antigen binding polypeptide complex described herein are joined by a cleavable linker, and VL2 and VL1 of an antigen binding polypeptide complex described herein are joined by a GS linker. In some aspects, VH1 and VL2 of an antigen binding polypeptide complex described herein are joined by a cleavable linker, and VH2 and VL1 of an antigen binding polypeptide complex described herein are joined by a GS linker. In some aspects, VL1 and VH2 of an antigen binding polypeptide complex described herein are joined by a cleavable linker, and VL2 and VH1 of an antigen binding polypeptide complex described herein are joined by a GS linker. In some aspects, VL1 and VL2 of an antigen binding polypeptide complex described herein are joined by a cleavable linker, and VH2 and VH1 of an antigen binding polypeptide complex described herein are joined by a GS linker. In some aspects, VH1 and VH2 of an antigen binding polypeptide complex described herein are joined by a GS linker, and VL2 and VL1 of an antigen binding polypeptide complex described herein are joined by a cleavable linker. In some aspects, VH1 and VL2 of an antigen binding polypeptide complex described herein are joined by a GS linker, and VH2 and VL1 of an antigen binding polypeptide complex described herein are joined by a cleavable linker. In some aspects, VL1 and VH2 of an antigen binding polypeptide complex described herein are joined by a GS linker, and VL2 and VH1 of an antigen binding polypeptide complex described herein are joined by a cleavable linker. In some aspects, VL1 and VL2 of an antigen binding polypeptide complex described herein are joined by a GS linker, and VH2 and VH1 of an antigen binding polypeptide complex described herein are joined by a cleavable linker.

In some aspects, VH3 and VH4 of an antigen binding polypeptide complex described herein are joined by a cleavable linker, and VL4 and VL3 of an antigen binding polypeptide complex described herein are joined by a GS linker. In some aspects, VH3 and VL4 of an antigen binding polypeptide complex described herein are joined by a cleavable linker, and VH4 and VL3 of an antigen binding polypeptide complex described herein are joined by a GS linker. In some aspects, VL3 and VH4 of an antigen binding polypeptide complex described herein are joined by a cleavable linker, and VL4 and VH3 of an antigen binding polypeptide complex described herein are joined by a GS linker. In some aspects, VL3 and VL4 of an antigen binding polypeptide complex described herein are joined by a cleavable linker, and VH4 and VH3 of an antigen binding polypeptide complex described herein are joined by a GS linker. In some aspects, VH3 and VH4 of an antigen binding polypeptide complex described herein are joined by a GS linker, and VL4 and VL3 of an antigen binding polypeptide complex described herein are joined by a cleavable linker. In some aspects, VH3 and VL4 of an antigen binding polypeptide complex described herein are joined by a GS linker, and VH4 and VL3 of an antigen binding polypeptide complex described herein are joined by a cleavable linker. In some aspects, VL3 and VH4 of an antigen binding polypeptide complex described herein are joined by a GS linker, and VL4 and VH3 of an antigen binding polypeptide complex described herein are joined by a cleavable linker. In some aspects, VL3 and VL4 of an antigen binding polypeptide complex described herein are joined by a GS linker, and VH4 and VH3 of an antigen binding polypeptide complex described herein are joined by a cleavable linker.

In some aspects, one or more of VH1, VH2, VH3 or VH4 of an antigen binding polypeptide complex described herein specifically binds to a tumor-associated antigen (TAA). In some aspects, one or more of VL1, VL2, VL3 or VL4 of an antigen binding polypeptide complex described herein specifically binds to a TAA. In some aspects, VH1 and VL1, VH2 and VL2, VH3 and VL3, and/or VH4 and VL4 or an antigen binding polypeptide complex described herein specifically binds to a TAA.

In some aspects, one or more of VH1, VH2, VH3 or VH4 of an antigen binding polypeptide complex described herein specifically binds to CD28. In some aspects, one or more of VL1, VL2, VL3 or VL4 of an antigen binding polypeptide complex described herein specifically binds to CD28. In some aspects, VH1 and VL1, VH2 and VL2, VH3 and VL3, and/or VH4 and VL4 or an antigen binding polypeptide complex described herein specifically binds to CD28.

In some aspects, the invention is directed to an antigen binding polypeptide complex comprising a first polypeptide, a second polypeptide, and a third polypeptide; wherein the first polypeptide has a structure represented by VL1-L1-VH1-L2-Fc; VH1-L3-VL1-L4-Fc; VH1-L1-VL1-L2-Fc; or VL1-L3-VH1-L4-Fc; wherein the second polypeptide has a structure represented by VH2-L5-VH3-L6-CH1-L7-Fc or VH3-L5-VH2-L6-CH1-L7-Fc; wherein the third polypeptide has a structure represented by VL2-L8-VL3-L9-CL or VL3-L8-VL2-L9-CL; wherein VL1 is a first immunoglobulin light chain variable region; VL2 is a second immunoglobulin light chain variable region; VL3 is a third immunoglobulin light chain variable region; VH1 is a first immunoglobulin heavy chain variable region; VH2 is a second immunoglobulin heavy chain variable region; VH3 is a third immunoglobulin heavy chain variable region; Fc is a region comprising an immunoglobulin heavy chain constant region 2 (CH2), an immunoglobulin heavy chain constant region 3 (CH3), and optionally, an immunoglobulin hinge; CH1 is an immunoglobulin heavy chain constant region 1; CL is an immunoglobulin light chain constant region; and L1-L9 are amino acid linkers. In some aspects, the first polypeptide has a structure represented by VL1-L1-VH1-L2-Fc; VH1-L3-VL1-L4-Fc; VH1-L1-VL1-L2-Fc; or VL1-L3-VH1-L4-Fc; the second polypeptide has a structure represented by VH2-L5-VH3-L6-CH1-L7-Fc; and the third polypeptide has a structure represented by VL2-L8-VL3-L9-CL. In some aspects, the first polypeptide has a structure represented by VL1-L1-VH1-L2-Fc; VH1-L3-VL1-L4-Fc; VH1-L1-VL1-L2-Fc; or VL1-L3-VH1-L4-Fc; the second polypeptide has a structure represented by VH2-L5-VH3-L6-CH1-L7-Fc; and the third polypeptide has a structure represented by VL3-L8-VL2-L9-CL. In some aspects, the first polypeptide has a structure represented by VL1-L1-VH1-L2-Fc; VH1-L3-VL1-L4-Fc; VH1-L1-VL1-L2-Fc; or VL1-L3-VH1-L4-Fc; the second polypeptide has a structure represented by VH3-L5-VH2-L6-CH1-L7-Fc, and the third polypeptide has a structure represented by VL2-L8-VL3-L9-CL. In some aspects, the first polypeptide has a structure represented by VL1-L1-VH1-L2-Fc; VH1-L3-VL1-L4-Fc; VH1-L1-VL1-L2-Fc; or VL1-L3-VH1-L4-Fc; the second polypeptide has a structure represented by VH3-L5-VH2-L6-CH1-L7-Fc; and the third polypeptide has a structure represented by VL3-L8-VL2-L9-CL. In some aspects, the invention is directed to an antigen binding polypeptide complex comprising a first polypeptide, a second polypeptide, and a third polypeptide; wherein the first polypeptide has a structure represented by VL1-L1-VH1-L2-Fc or VH1-L3-VL1-L4-Fc; wherein the second polypeptide has a structure represented by VH2-L5-VH3-L6-CH1-L7-Fc; wherein the third polypeptide has a structure represented by VL2-L8-VL3-L9-CL; wherein VL1 is a first immunoglobulin light chain variable region; VL2 is a second immunoglobulin light chain variable region; VL3 is a third immunoglobulin light chain variable region; VH1 is a first immunoglobulin heavy chain variable region; VH2 is a second immunoglobulin heavy chain variable region; VH3 is a third immunoglobulin heavy chain variable region; Fc is a region comprising an immunoglobulin heavy chain constant region 2 (CH2), an immunoglobulin heavy chain constant region 3 (CH3), and optionally, an immunoglobulin hinge; CH1 is an immunoglobulin heavy chain constant region 1; CL is an immunoglobulin light chain constant region; and L1-L9 are amino acid linkers.

21| In some aspects, the invention is directed to an antigen binding polypeptide complex comprising a first polypeptide, a second polypeptide, a third polypeptide, and a fourth polypeptide; wherein the first polypeptide has a structure represented by VL1-L1-CL or VH1-L1-CL; wherein the second polypeptide has a structure represented by VH1-L2-CH1-L3-Fc or VL1-L2-CH1-L3-Fc; wherein the third polypeptide has a structure represented by VH2-L4-VH3-L5-CH1-L6-Fc or VH3-L4-VH2-L5-CH1-L6-Fc; wherein the fourth polypeptide has a structure represented by VL2-L7-VL3-L8-CL or VL3-L7-VL2-L8-CL; wherein VL1 is a first immunoglobulin light chain variable region; VL2 is a second immunoglobulin light chain variable region; VL3 is a third immunoglobulin light chain variable region; VH1 is a first immunoglobulin heavy chain variable region; VH2 is a second immunoglobulin heavy chain variable region; VH3 is a third immunoglobulin heavy chain variable region; Fc is a region comprising an immunoglobulin heavy chain constant region 2 (CH2), an immunoglobulin heavy chain constant region 3 (CH3), and optionally, an immunoglobulin hinge; CH1 is an immunoglobulin heavy chain constant region 1; CL is an immunoglobulin light chain constant region; and L1-L8 are amino acid linkers. In some aspects, the invention is directed to an antigen binding polypeptide complex comprising a first polypeptide, a second polypeptide, a third polypeptide, and a fourth polypeptide; wherein the first polypeptide has a structure represented by VL1-L1-CL; wherein the second polypeptide has a structure represented by VH1-L2-CH1-L3-Fc; wherein the third polypeptide has a structure represented by VH2-L4-VH3-L5-CH1-L6-Fc or VH3-L4-VH2-L5-CH1-L6-Fc; wherein the fourth polypeptide has a structure represented by VL2-L7-VL3-L8-CL or VL3-L7-VL2-L8-CL; wherein VL1 is a first immunoglobulin light chain variable region; VL2 is a second immunoglobulin light chain variable region; VL3 is a third immunoglobulin light chain variable region; VH1 is a first immunoglobulin heavy chain variable region; VH2 is a second immunoglobulin heavy chain variable region; VH3 is a third immunoglobulin heavy chain variable region; Fc is a region comprising an immunoglobulin heavy chain constant region 2 (CH2), an immunoglobulin heavy chain constant region 3 (CH3), and optionally, an immunoglobulin hinge; CH1 is an immunoglobulin heavy chain constant region 1; CL is an immunoglobulin light chain constant region; and L1-L8 are amino acid linkers. In some aspects, the invention is directed to an antigen binding polypeptide complex comprising a first polypeptide, a second polypeptide, a third polypeptide, and a fourth polypeptide; wherein the first polypeptide has a structure represented by VH1-L1-CL; wherein the second polypeptide has a structure represented by VL1-L2-CH1-L3-Fc; wherein the third polypeptide has a structure represented by VH2-L4-VH3-L5-CH1-L6-Fc or VH3-L4-VH2-L5-CH1-L6-Fc; wherein the fourth polypeptide has a structure represented by VL2-L7-VL3-L8-CL or VL3-L7-VL2-L8-CL; wherein VL1 is a first immunoglobulin light chain variable region; VL2 is a second immunoglobulin light chain variable region; VL3 is a third immunoglobulin light chain variable region; VH1 is a first immunoglobulin heavy chain variable region; VH2 is a second immunoglobulin heavy chain variable region; VH3 is a third immunoglobulin heavy chain variable region; Fc is a region comprising an immunoglobulin heavy chain constant region 2 (CH2), an immunoglobulin heavy chain constant region 3 (CH3), and optionally, an immunoglobulin hinge; CH1 is an immunoglobulin heavy chain constant region 1; CL is an immunoglobulin light chain constant region; and L1-L8 are amino acid linkers. In some aspects, the first polypeptide has a structure represented by VL1-L1-CL; the second polypeptide has a structure represented by VH1-L2-CH1-L3-Fc; the third polypeptide has a structure represented by VH2-L4-VH3-L5-CH1-L6-Fc; and the fourth polypeptide has a structure represented by VL2-L7-VL3-L8-CL. In some aspects, the first polypeptide has a structure represented by VL1-L1-CL; the second polypeptide has a structure represented by VH1-L2-CH1-L3-Fc; the third polypeptide has a structure represented by VH3-L4-VH2-L5-CH1-L6-Fc; and the fourth polypeptide has a structure represented by VL2-L7-VL3-L8-CL. In some aspects, the first polypeptide has a structure represented by VL1-L1-CL; the second polypeptide has a structure represented by VH1-L2-CH1-L3-Fc; the third polypeptide has a structure represented by VH2-L4-VH3-L5-CH1-L6-Fc; and the fourth polypeptide has a structure represented by VL3-L7-VL2-L8-CL. In some aspects, the first polypeptide has a structure represented by VL1-L1-CL; the second polypeptide has a structure represented by VH1-L2-CH1-L3-Fc; the third polypeptide has a structure represented by VH3-L4-VH2-L5-CH1-L6-Fc; and the fourth polypeptide has a structure represented by VL3-L7-VL2-L8-CL. In some aspects, the first polypeptide has a structure represented by VH1-L1-CL; the second polypeptide has a structure represented by VL1-L2-CH1-L3-Fc; the third polypeptide has a structure represented by VH2-L4-VH3-L5-CH1-L6-Fc; and the fourth polypeptide has a structure represented by VL2-L7-VL3-L8-CL. In some aspects, the first polypeptide has a structure represented by VH1-L1-CL; the second polypeptide has a structure represented by VL1-L2-CH1-L3-Fc; the third polypeptide has a structure represented by VH3-L4-VH2-L5-CH1-L6-Fc; and the fourth polypeptide has a structure represented by VL2-L7-VL3-L8-CL. In some aspects, the first polypeptide has a structure represented by VH1-L1-CL; the second polypeptide has a structure represented by VL1-L2-CH1-L3-Fc; the third polypeptide has a structure represented by VH2-L4-VH3-L5-CH1-L6-Fc; and the fourth polypeptide has a structure represented by VL3-L7-VL2-L8-CL. In some aspects, the first polypeptide has a structure represented by VH1-L1-CL; the second polypeptide has a structure represented by VL1-L2-CH1-L3-Fc; the third polypeptide has a structure represented by VH3-L4-VH4-L5-CH1-L6-Fc; and the fourth polypeptide has a structure represented by VL3-L7-VL2-L8-CL.

In some aspects, the invention is directed to an antigen binding polypeptide complex comprising a first polypeptide, a second polypeptide, and a third polypeptide; wherein the first polypeptide has a structure represented by VL1-L1-VL2-L2-VH2-L3-VH1-L4-Fc; VL1-L1-VH2-L2-VL2-L3-VH1-L4-Fc; VH1-L5-VH2-L6-VL2-L7-VL1-L8-Fc; or VH1-L5-VL2-L6-VH2-L7-VL1-L8-Fc; wherein the second polypeptide has a structure represented by VH3-L9-VH4-L10-CH1-L11-Fc or VH4-L9-VH3-L10-CH1-L11-Fc; wherein the third polypeptide has a structure represented by VL3-L12-VL4-L13-CL or VL4-L12-VL3-L13-CL; wherein VL1 is a first immunoglobulin light chain variable region; VL2 is a second immunoglobulin light chain variable region; VL3 is a third immunoglobulin light chain variable region; VL4 is a fourth immunoglobulin light chain variable region; VH1 is a first immunoglobulin heavy chain variable region; VH2 is a second immunoglobulin heavy chain variable region; VH3 is a third immunoglobulin heavy chain variable region; VH4 is a fourth immunoglobulin heavy chain variable region; Fc is a region comprising an immunoglobulin heavy chain constant region 2 (CH2), an immunoglobulin heavy chain constant region 3 (CH3), and optionally, an immunoglobulin hinge; CH1 is an immunoglobulin heavy chain constant region 1; CL is an immunoglobulin light chain constant region; and L1-L13 are amino acid linkers. In some aspects, the first polypeptide has a structure represented by VL1-L1-VL2-L2-VH2-L3-VH1-L4-Fc; VL1-L1-VH2-L2-VL2-L3-VH1-L4-Fc; VH1-L5-VH2-L6-VL2-L7-VL1-L8-Fc; or VH1-L5-VL2-L6-VH2-L7-VL1-L8-Fc; the second polypeptide has a structure represented by VH3-L9-VH4-L10-CH1-L11-Fc, and the third polypeptide has a structure represented by VL3-L12-VL4-L13-CL. In some aspects, the first polypeptide has a structure represented by VL1-L1-VL2-L2-VH2-L3-VH1-L4-Fc; VL1-L1-VH2-L2-VL2-L3-VH1-L4-Fc; VH1-L5-VH2-L6-VL2-L7-VL1-L8-Fc; or VH1-L5-VL2-L6-VH2-L7-VL1-L8-Fc; the second polypeptide has a structure represented by VH3-L9-VH4-L10-CH1-L11-Fc; and the third polypeptide has a structure represented by VL4-L12-VL3-L13-CL. In some aspects, the first polypeptide has a structure represented by VL1-L1-VL2-L2-VH2-L3-VH1-L4-Fc; VL1-L1-VH2-L2-VL2-L3-VH1-L4-Fc; VH1-L5-VH2-L6-VL2-L7-VL1-L8-Fc; or VH1-L5-VL2-L6-VH2-L7-VL1-L8-Fc; the second polypeptide has a structure represented by VH4-L9-VH3-L10-CH1-L1 I-Fc, and the third polypeptide has a structure represented by VL3-L12-VL4-L13-CL. In some aspects, the first polypeptide has a structure represented by VL1-L1-VL2-L2-VH2-L3-VH1-L4-Fc; VL1-L1-VH2-L2-VL2-L3-VH1-L4-Fc; VH1-L5-VH2-L6-VL2-L7-VL1-L8-Fc; or VH1-L5-VL2-L6-VH2-L7-VL1-L8-Fc; the second polypeptide has a structure represented by VH4-L9-VH3-L10-CH1-L11-Fc; and the third polypeptide has a structure represented by VL4-L12-VL3-L13-CL. In some aspects, the first polypeptide has a structure represented by: VL1-L1-VL2-L2-VH2-L3-VH1-L4-Fc; or VH1-L5-VH2-L6-VL2-L7-VL1-L8-Fc; the second polypeptide has a structure represented by: VH3-L9-VH4-L10-CH1-L11-Fc; and the third polypeptide has a structure represented by: VL3-L12-VL4-L13-CL. In some aspects, the first polypeptide has a structure represented by: VL1-L1-VL2-L2-VH2-L3-VH1-L4-Fc or VH1-L5-VL2-L6-VH2-L7-VL1-L8-Fc; the second polypeptide has a structure represented by: VH3-L9-VH4-L10-CH1-L11-Fc; and the third polypeptide has a structure represented by: VL3-L12-VL4-L13-CL. In some aspects, the first polypeptide has a structure represented by: VL1-L1-VL2-L2-VH2-L3-VH1-L4-Fc; the second polypeptide has a structure represented by: VH3-L9-VH4-L10-CH1-L11-Fc; and the third polypeptide has a structure represented by: VL3-L12-VL4-L13-CL. In some aspects, the first polypeptide has a structure represented by: VH1-L5-VL2-L6-VH2-L7-VL1-L8-Fc; the second polypeptide has a structure represented by: VH3-L9-VH4-L10-CH1-L11-Fc; and the third polypeptide has a structure represented by: VL3-L12-VL4-L13-CL.

In some aspects, the invention is directed to an antigen binding polypeptide complex comprising a first polypeptide, a second polypeptide, and a third polypeptide; wherein the first polypeptide has a structure represented by VL1-L1-VL2-L2-VH2-L3-VH1-L4-Fc; VL1-L1-VH2-L2-VL2-L3-VH1-L4-Fc; VH1-L5-VH2-L6-VL2-L7-VL1-L8-Fc; or VH1-L5-VL2-L6-VH2-L7-VL1-L8-Fc; wherein the second polypeptide has a structure represented by VH3-L9-VH4-CH1-Fc; or VH4-L9-VH3-CH1-Fc; wherein the third polypeptide has a structure represented by VL3-L10-VL4-CL or VL4-L10-VL3-CL; wherein VL1 is a first immunoglobulin light chain variable region; VL2 is a second immunoglobulin light chain variable region; VL3 is a third immunoglobulin light chain variable region; VL4 is a fourth immunoglobulin light chain variable region; VH1 is a first immunoglobulin heavy chain variable region; VH2 is a second immunoglobulin heavy chain variable region; VH3 is a third immunoglobulin heavy chain variable region; VH4 is a fourth immunoglobulin heavy chain variable region; Fc is a region comprising an immunoglobulin heavy chain constant region 2 (CH2), an immunoglobulin heavy chain constant region 3 (CH3), and optionally, an immunoglobulin hinge; CH1 is an immunoglobulin heavy chain constant region 1; CL is an immunoglobulin light chain constant region; and L1-L10 are amino acid linkers. In some aspects, the first polypeptide has a structure represented by VL1-L1-VL2-L2-VH2-L3-VH1-L4-Fc; VL1-L1-VH2-L2-VL2-L3-VH1-L4-Fc; VH1-L5-VH2-L6-VL2-L7-VL1-L8-Fc; or VH1-L5-VL2-L6-VH2-L7-VL1-L8-Fc; the second polypeptide has a structure represented by VH3-L9-VH4-CH1-Fc, and the third polypeptide has a structure represented by VL3-L10-VL4-CL. In some aspects, the first polypeptide has a structure represented by VL1-L1-VL2-L2-VH2-L3-VH1-L4-Fc; VL1-L1-VH2-L2-VL2-L3-VH1-L4-Fc; VH1-L5-VH2-L6-VL2-L7-VL1-L8-Fc; or VH1-L5-VL2-L6-VH2-L7-VL1-L8-Fc; the second polypeptide has a structure represented by VH3-L9-VH4-CH1-Fc; and the third polypeptide has a structure represented by VL4-L10-VL3-CL. In some aspects, the first polypeptide has a structure represented by VL1-L1-VL2-L2-VH2-L3-VH1-L4-Fc; VL1-L1-VH2-L2-VL2-L3-VH1-L4-Fc; VH1-L5-VH2-L6-VL2-L7-VL1-L8-Fc; or VH1-L5-VL2-L6-VH2-L7-VL1-L8-Fc; the second polypeptide has a structure represented by VH4-L9-VH3-CH1-Fc, and the third polypeptide has a structure represented by VL3-L10-VL4-CL. In some aspects, the first polypeptide has a structure represented by VL1-L1-VL2-L2-VH2-L3-VH1-L4-Fc; VL1-L1-VH2-L2-VL2-L3-VH1-L4-Fc; VH1-L5-VH2-L6-VL2-L7-VL1-L8-Fc; or VH1-L5-VL2-L6-VH2-L7-VL1-L8-Fc; the second polypeptide has a structure represented by VH4-L9-VH3-CH1-Fc; and the third polypeptide has a structure represented by VL4-L10-VL3-CL. In some aspects, the first polypeptide has a structure represented by: VL1-L1-VL2-L2-VH2-L3-VH1-L4-Fc; or VH1-L5-VH2-L6-VL2-L7-VL1-L8-Fc; the second polypeptide has a structure represented by: VH3-L9-VH4-CH1-Fc; and the third polypeptide has a structure represented by: VL3-L10-VL4-CL. In some aspects, the first polypeptide has a structure represented by: VL1-L1-VL2-L2-VH2-L3-VH1-L4-Fc or VH1-L5-VL2-L6-VH2-L7-VL1-L8-Fc; the second polypeptide has a structure represented by: VH3-L9-VH4-CH1-Fc; and the third polypeptide has a structure represented by: VL3-L10-VL4-CL. In some aspects, the first polypeptide has a structure represented by: VL1-L1-VL2-L2-VH2-L3-VH1-L4-Fc; the second polypeptide has a structure represented by: VH3-L9-VH4-CH1-Fc; and the third polypeptide has a structure represented by: VL3-L10-VL4-CL. In some aspects, the first polypeptide has a structure represented by: VH1-L5-VL2-L6-VH2-L7-VL1-L8-Fc; the second polypeptide has a structure represented by: VH3-L9-VH4-CH1-Fc; and the third polypeptide has a structure represented by: VL3-L10-VL4-CL.

In some aspects, the invention is directed to an antigen binding polypeptide complex comprising a first polypeptide and a second polypeptide; wherein the first polypeptide has a structure represented by VL1-L1-VL2-L2-VH2-L3-VH1-L4-Fc; VL1-L1-VH2-L2-VL2-L3-VH1-L4-Fc; VH1-L5-VH2-L6-VL2-L7-VL1-L8-Fc; or VH1-L5-VL2-L6-VH2-VL1-L8-Fc; wherein the second polypeptide has a structure represented by VL3-L9-VL4-L10-VH4-L11-VH3-L12-Fc; VL3-L9-VH4-L10-VL4-L11-VH3-L12-Fc; VH3-L13-VH4-L14-VL4-L15-VL3-L16-Fc; or VH3-L13-VL4-L14-VH4-L15-VL3-L16-Fc; wherein VL1 is a first immunoglobulin light chain variable region that specifically binds to a tumor-associated antigen; VL2 is a second immunoglobulin light chain variable region that specifically binds to a tumor-associated antigen; VL3 is a third immunoglobulin light chain variable region that specifically binds to CD3; VL4 is a fourth immunoglobulin light chain variable region that specifically binds to a tumor-associated antigen; VH1 is a first immunoglobulin heavy chain variable region that specifically binds to a tumor-associated antigen; VH2 is a second immunoglobulin heavy chain variable region that specifically binds to a tumor-associated antigen; VH3 is a third immunoglobulin heavy chain variable region that specifically binds to CD3; VH4 is a fourth immunoglobulin heavy chain variable region that specifically binds to a tumor-associated antigen; Fc is a region comprising an immunoglobulin heavy chain constant region 2 (CH2), an immunoglobulin heavy chain constant region 3 (CH3), and optionally, an immunoglobulin hinge; and L1-L16 are amino acid linkers. In some aspects, the first polypeptide has a structure represented by VL1-L1-VL2-L2-VH2-L3-VH1-L4-Fc; VL1-L1-VH2-L2-VL2-L3-VH1-L4-Fc; VH1-L5-VH2-L6-VL2-L7-VL1-L8-Fc; or VH1-L5-VL2-L6-VH2-VL1-L8-Fc; and the second polypeptide has a structure represented by VL3-L9-VL4-L10-VH4-L11-VH3-L12-Fc. In some aspects, the first polypeptide has a structure represented by VL1-L1-VL2-L2-VH2-L3-VH1-L4-Fc; VL1-L1-VH2-L2-VL2-L3-VH1-L4-Fc; VH1-L5-VH2-L6-VL2-L7-VL1-L8-Fc; or VH1-L5-VL2-L6-VH2-VL1-L8-Fc; and the second polypeptide has a structure represented by VL3-L9-VH4-L10-VL4-L11-VH3-L12-Fc. In some aspects, the first polypeptide has a structure represented by VL1-L1-VL2-L2-VH2-L3-VH1-L4-Fc; VL1-L1-VH2-L2-VL2-L3-VH1-L4-Fc; VH1-L5-VH2-L6-VL2-L7-VL1-L8-Fc; or VH1-L5-VL2-L6-VH2-VL1-L8-Fc; and the second polypeptide has a structure represented by VH3-L13-VH4-L14-VL4-L15-VL3-L16-Fc. In some aspects, the first polypeptide has a structure represented by VL1-L1-VL2-L2-VH2-L3-VH1-L4-Fc; VL1-L1-VH2-L2-VL2-L3-VH1-L4-Fc; VH1-L5-VH2-L6-VL2-L7-VL1-L8-Fc; or VH1-L5-VL2-L6-VH2-VL1-L8-Fc; and the second polypeptide has a structure represented by VH3-L13-VL4-L14-VH4-L15-VL3-L16-Fc. In some aspects, the first polypeptide has a structure represented by VL1-L1-VL2-L2-VH2-L3-VH1-L4-Fc or VH1-L5-VH2-L6-VL2-L7-VL1-L8-Fc; and the second polypeptide has a structure represented by VL3-L9-VL4-L10-VH4-L11-VH3-L12-Fc or VH3-L13-VH4-L14-VL4-L15-VL3-L16-Fc. In some aspects, the first polypeptide has a structure represented by VL1-L1-VL2-L2-VH2-L3-VH1-L4-Fc and the second polypeptide has a structure represented by VL3-L9-VL4-L10-VH4-L11-VH3-L12-Fc. In some aspects, the first polypeptide has a structure represented by VL1-L1-VL2-L2-VH2-L3-VH1-L4-Fc and the second polypeptide has a structure represented by VH3-L13-VH4-L14-VL4-L15-VL3-L16-Fc. In some aspects, the first polypeptide has a structure represented by VH1-L5-VH2-L6-VL2-L7-VL1-L8-Fc and the second polypeptide has a structure represented by VL3-L9-VL4-L10-VH4-L11-VH3-L12-Fc. In some aspects, the first polypeptide has a structure represented by VH1-L5-VH2-L6-VL2-L7-VL1-L8-Fc and the second polypeptide has a structure represented by VH3-L13-VH4-L14-VL4-L15-VL3-L16-Fc.

In some aspects, the first polypeptide has a structure represented by VL1-L1-VL2-L2-VH2-L3-VH1-L4-Fc and the second polypeptide has a structure represented by VL3-L5-VL4-L6-VH4-L7-VH3-L8-Fc; wherein VL1 is a first immunoglobulin light chain variable region that specifically binds to Trop2, cMet, CD3 or CD28; VL2 is a second immunoglobulin light chain variable region that specifically binds to Trop2, cMet, CD3 or CD28; VL3 is a third immunoglobulin light chain variable region that specifically binds to Trop2, cMet, CD3 or CD28; VL4 is a fourth immunoglobulin light chain variable region that specifically binds to Trop2, cMet, CD3 or CD28; VH1 is a first immunoglobulin heavy chain variable region that specifically binds to Trop2, cMet, CD3 or CD28; VH2 is a second immunoglobulin heavy chain variable region that specifically binds to Trop2, cMet, CD3 or CD28; VH3 is a third immunoglobulin heavy chain variable region that specifically binds to Trop2, cMet, CD3 or CD28; VH4 is a fourth immunoglobulin heavy chain variable region that specifically binds to Trop2, cMet, CD3 or CD28; Fc is a region comprising an immunoglobulin heavy chain constant region 2 (CH2), an immunoglobulin heavy chain constant region 3 (CH3), and optionally, an immunoglobulin hinge; and L1-L8 are amino acid linkers. For example, VL1 is a first immunoglobulin light chain variable region that specifically binds to Trop2; VL2 is a second immunoglobulin light chain variable region that specifically binds to cMet; VL3 is a third immunoglobulin light chain variable region that specifically binds to CD3; VL4 is a fourth immunoglobulin light chain variable region that specifically binds to CD28; VH1 is a first immunoglobulin heavy chain variable region that specifically binds to Trop2; VH2 is a second immunoglobulin heavy chain variable region that specifically binds to cMet; VH3 is a third immunoglobulin heavy chain variable region that specifically binds to CD3; and VH4 is a fourth immunoglobulin heavy chain variable region that specifically binds to CD28. For example, VL1 is a first immunoglobulin light chain variable region that specifically binds to Trop2; VL2 is a second immunoglobulin light chain variable region that specifically binds to cMet; VL3 is a third immunoglobulin light chain variable region that specifically binds to CD28; VL4 is a fourth immunoglobulin light chain variable region that specifically binds to CD3; VH1 is a first immunoglobulin heavy chain variable region that specifically binds to Trop2; VH2 is a second immunoglobulin heavy chain variable region that specifically binds to cMet; VH3 is a third immunoglobulin heavy chain variable region that specifically binds to CD28; and VH4 is a fourth immunoglobulin heavy chain variable region that specifically binds to CD3. For example, VL1 is a first immunoglobulin light chain variable region that specifically binds to cMet; VL2 is a second immunoglobulin light chain variable region that specifically binds to Trop2; VL3 is a third immunoglobulin light chain variable region that specifically binds to CD3; VL4 is a fourth immunoglobulin light chain variable region that specifically binds to CD28; VH1 is a first immunoglobulin heavy chain variable region that specifically binds to cMet; VH2 is a second immunoglobulin heavy chain variable region that specifically binds to Trop2; VH3 is a third immunoglobulin heavy chain variable region that specifically binds to CD3; and VH4 is a fourth immunoglobulin heavy chain variable region that specifically binds to CD28. For example, VL1 is a first immunoglobulin light chain variable region that specifically binds to cMet; VL2 is a second immunoglobulin light chain variable region that specifically binds to Trop2; VL3 is a third immunoglobulin light chain variable region that specifically binds to CD28; VL4 is a fourth immunoglobulin light chain variable region that specifically binds to CD3; VH1 is a first immunoglobulin heavy chain variable region that specifically binds to cMet; VH2 is a second immunoglobulin heavy chain variable region that specifically binds to Trop2; VH3 is a third immunoglobulin heavy chain variable region that specifically binds to CD28; and VH4 is a fourth immunoglobulin heavy chain variable region that specifically binds to CD3. In some aspects, the antigen binding polypeptide complex comprises amino acid sequences having at least 80%, at least 85%, at least 90%, at least 95%, or 100% identity to SEQ ID NOs:204 and 511. In some aspects, the antigen binding polypeptide complex comprises amino acid sequences encoded by polynucleotides having at least 80%, at least 85%, at least 90%, at least 95%, or 100% identity to SEQ ID NOs:205 and 512.

In some aspects, the first polypeptide has a structure represented by VL1-L1-VH2-L2-VL2-L3-VH1-L4-Fc and the second polypeptide has a structure represented by VL3-L5-VH4-L6-VL4-L7-VH3-L8-Fc; wherein VL1 is a first immunoglobulin light chain variable region that specifically binds to Trop2, cMet, CD3 or CD28; VL2 is a second immunoglobulin light chain variable region that specifically binds to Trop2, cMet, CD3 or CD28; VL3 is a third immunoglobulin light chain variable region that specifically binds to Trop2, cMet, CD3 or CD28; VL4 is a fourth immunoglobulin light chain variable region that specifically binds to Trop2, cMet, CD3 or CD28; VH1 is a first immunoglobulin heavy chain variable region that specifically binds to Trop2, cMet, CD3 or CD28; VH2 is a second immunoglobulin heavy chain variable region that specifically binds to Trop2, cMet, CD3 or CD28; VH3 is a third immunoglobulin heavy chain variable region that specifically binds to Trop2, cMet, CD3 or CD28; VH4 is a fourth immunoglobulin heavy chain variable region that specifically binds to Trop2, cMet, CD3 or CD28; Fc is a region comprising an immunoglobulin heavy chain constant region 2 (CH2), an immunoglobulin heavy chain constant region 3 (CH3), and optionally, an immunoglobulin hinge; and L1-L8 are amino acid linkers. For example, VL1 is a first immunoglobulin light chain variable region that specifically binds to cMet; VL2 is a second immunoglobulin light chain variable region that specifically binds to CD28; VL3 is a third immunoglobulin light chain variable region that specifically binds to Trop2; VL4 is a fourth immunoglobulin light chain variable region that specifically binds to CD3; VH1 is a first immunoglobulin heavy chain variable region that specifically binds to cMet; VH2 is a second immunoglobulin heavy chain variable region that specifically binds to CD28; VH3 is a third immunoglobulin heavy chain variable region that specifically binds to Trop2; and VH4 is a fourth immunoglobulin heavy chain variable region that specifically binds to CD3. In some aspects, the antigen binding polypeptide complex comprises amino acid sequences having at least 80%, at least 85%, at least 90%, at least 95%, or 100% identity to SEQ ID NOs:634 and 652. In some aspects, the antigen binding polypeptide complex comprises amino acid sequences encoded by polynucleotides having at least 80%, at least 85%, at least 90%, at least 95%, or 100% identity to SEQ ID NOs:635 and 653.

In some aspects, the first polypeptide has a structure represented by VH1-L1-VL2-L2-VH2-L3-VL1-L4-Fc and the second polypeptide has a structure represented by VH3-L5-VL4-L6-VH4-L7-VL3-L8-Fc; wherein VL1 is a first immunoglobulin light chain variable region that specifically binds to Trop2, cMet, CD3 or CD28; VL2 is a second immunoglobulin light chain variable region that specifically binds to Trop2, cMet, CD3 or CD28; VL3 is a third immunoglobulin light chain variable region that specifically binds to Trop2, cMet, CD3 or CD28; VL4 is a fourth immunoglobulin light chain variable region that specifically binds to Trop2, cMet, CD3 or CD28; VH1 is a first immunoglobulin heavy chain variable region that specifically binds to Trop2, cMet, CD3 or CD28; VH2 is a second immunoglobulin heavy chain variable region that specifically binds to Trop2, cMet, CD3 or CD28; VH3 is a third immunoglobulin heavy chain variable region that specifically binds to Trop2, cMet, CD3 or CD28; VH4 is a fourth immunoglobulin heavy chain variable region that specifically binds to Trop2, cMet, CD3 or CD28; Fc is a region comprising an immunoglobulin heavy chain constant region 2 (CH2), an immunoglobulin heavy chain constant region 3 (CH3), and optionally, an immunoglobulin hinge; and L1-L8 are amino acid linkers. For example, VL1 is a first immunoglobulin light chain variable region that specifically binds to cMet; VL2 is a second immunoglobulin light chain variable region that specifically binds to CD28; VL3 is a third immunoglobulin light chain variable region that specifically binds to Trop2; VL4 is a fourth immunoglobulin light chain variable region that specifically binds to CD3; VH1 is a first immunoglobulin heavy chain variable region that specifically binds to cMet; VH2 is a second immunoglobulin heavy chain variable region that specifically binds to CD28; VH3 is a third immunoglobulin heavy chain variable region that specifically binds to Trop2; and VH4 is a fourth immunoglobulin heavy chain variable region that specifically binds to CD3. In some aspects, the antigen binding polypeptide complex comprises amino acid sequences having at least 80%, at least 85%, at least 90%, at least 95%, or 100% identity to SEQ ID NOs:670 and 688. In some aspects, the antigen binding polypeptide complex comprises amino acid sequences encoded by polynucleotides having at least 80%, at least 85%, at least 90%, at least 95%, or 100% identity to SEQ ID NOs:671 and 689.

In some aspects, the first polypeptide has a structure represented by VH1-L1-VH2-L2-VL2-L3-VL1-L4-Fc and the second polypeptide has a structure represented by VH3-L5-VH4-L6-VL4-L7-VL3-L8-Fc; wherein VL1 is a first immunoglobulin light chain variable region that specifically binds to Trop2, cMet, CD3 or CD28; VL2 is a second immunoglobulin light chain variable region that specifically binds to Trop2, cMet, CD3 or CD28; VL3 is a third immunoglobulin light chain variable region that specifically binds to Trop2, cMet, CD3 or CD28; VL4 is a fourth immunoglobulin light chain variable region that specifically binds to Trop2, cMet, CD3 or CD28; VH1 is a first immunoglobulin heavy chain variable region that specifically binds to Trop2, cMet, CD3 or CD28; VH2 is a second immunoglobulin heavy chain variable region that specifically binds to Trop2, cMet, CD3 or CD28; VH3 is a third immunoglobulin heavy chain variable region that specifically binds to Trop2, cMet, CD3 or CD28; VH4 is a fourth immunoglobulin heavy chain variable region that specifically binds to Trop2, cMet, CD3 or CD28; Fc is a region comprising an immunoglobulin heavy chain constant region 2 (CH2), an immunoglobulin heavy chain constant region 3 (CH3), and optionally, an immunoglobulin hinge; and L1-L8 are amino acid linkers. For example, VL1 is a first immunoglobulin light chain variable region that specifically binds to cMet; VL2 is a second immunoglobulin light chain variable region that specifically binds to CD28; VL3 is a third immunoglobulin light chain variable region that specifically binds to Trop2; VL4 is a fourth immunoglobulin light chain variable region that specifically binds to CD3; VH1 is a first immunoglobulin heavy chain variable region that specifically binds to cMet; VH2 is a second immunoglobulin heavy chain variable region that specifically binds to CD28; VH3 is a third immunoglobulin heavy chain variable region that specifically binds to Trop2; and VH4 is a fourth immunoglobulin heavy chain variable region that specifically binds to CD3. In some aspects, the antigen binding polypeptide complex comprises amino acid sequences having at least 80%, at least 85%, at least 90%, at least 95%, or 100% identity to SEQ ID NOs:706 and 724. In some aspects, the antigen binding polypeptide complex comprises amino acid sequences encoded by polynucleotides having at least 80%, at least 85%, at least 90%, at least 95%, or 100% identity to SEQ ID NOs:707 and 725.

In some aspects, the first polypeptide has a structure represented by VL1-L1-VL2-L2-VH2-L3-VH1-L4-Fc and the second polypeptide has a structure represented by VL3-L5-VL4-L6-VH4-L7-VH3-L8-Fc; wherein VL1 is a first immunoglobulin light chain variable region that specifically binds to CD19, CD20, CD3 or CD28; VL2 is a second immunoglobulin light chain variable region that specifically binds to CD19, CD20, CD3 or CD28; VL3 is a third immunoglobulin light chain variable region that specifically binds to CD19, CD20, CD3 or CD28; VL4 is a fourth immunoglobulin light chain variable region that specifically binds to CD19, CD20, CD3 or CD28; VH1 is a first immunoglobulin heavy chain variable region that specifically binds to CD19, CD20, CD3 or CD28; VH2 is a second immunoglobulin heavy chain variable region that specifically binds to CD19, CD20, CD3 or CD28; VH3 is a third immunoglobulin heavy chain variable region that specifically binds to CD19, CD20, CD3 or CD28; VH4 is a fourth immunoglobulin heavy chain variable region that specifically binds to CD19, CD20, CD3 or CD28; Fc is a region comprising an immunoglobulin heavy chain constant region 2 (CH2), an immunoglobulin heavy chain constant region 3 (CH3), and optionally, an immunoglobulin hinge; and L1-L8 are amino acid linkers. For example, VL1 is a first immunoglobulin light chain variable region that specifically binds to CD19; VL2 is a second immunoglobulin light chain variable region that specifically binds to CD20; VL3 is a third immunoglobulin light chain variable region that specifically binds to CD3; VL4 is a fourth immunoglobulin light chain variable region that specifically binds to CD28; VH1 is a first immunoglobulin heavy chain variable region that specifically binds to CD19; VH2 is a second immunoglobulin heavy chain variable region that specifically binds to CD20; VH3 is a third immunoglobulin heavy chain variable region that specifically binds to CD3; and VH4 is a fourth immunoglobulin heavy chain variable region that specifically binds to CD28. For example, VL1 is a first immunoglobulin light chain variable region that specifically binds to CD20; VL2 is a second immunoglobulin light chain variable region that specifically binds to CD19; VL3 is a third immunoglobulin light chain variable region that specifically binds to CD3; VL4 is a fourth immunoglobulin light chain variable region that specifically binds to CD28; VH1 is a first immunoglobulin heavy chain variable region that specifically binds to CD20; VH2 is a second immunoglobulin heavy chain variable region that specifically binds to CD19; VH3 is a third immunoglobulin heavy chain variable region that specifically binds to CD3; and VH4 is a fourth immunoglobulin heavy chain variable region that specifically binds to CD28. For example, VL1 is a first immunoglobulin light chain variable region that specifically binds to CD19; VL2 is a second immunoglobulin light chain variable region that specifically binds to CD20; VL3 is a third immunoglobulin light chain variable region that specifically binds to CD28; VL4 is a fourth immunoglobulin light chain variable region that specifically binds to CD3; VH1 is a first immunoglobulin heavy chain variable region that specifically binds to CD19; VH2 is a second immunoglobulin heavy chain variable region that specifically binds to CD20; VH3 is a third immunoglobulin heavy chain variable region that specifically binds to CD28; and VH4 is a fourth immunoglobulin heavy chain variable region that specifically binds to CD3. For example, VL1 is a first immunoglobulin light chain variable region that specifically binds to CD20; VL2 is a second immunoglobulin light chain variable region that specifically binds to a CD19; VL3 is a third immunoglobulin light chain variable region that specifically binds to CD28; VL4 is a fourth immunoglobulin light chain variable region that specifically binds to CD3; VH1 is a first immunoglobulin heavy chain variable region that specifically binds to CD20; VH2 is a second immunoglobulin heavy chain variable region that specifically binds to CD19; VH3 is a third immunoglobulin heavy chain variable region that specifically binds to CD28; and VH4 is a fourth immunoglobulin heavy chain variable region that specifically binds to CD3.

In some aspects, the first polypeptide has a structure represented by VL1-L1-VL2-L2-VH2-L3-VH1-L4-Fc and the second polypeptide has a structure represented by VL3-L5-VL4-L6-VH4-L7-VH3-L8-Fc; wherein VL1 is a first immunoglobulin light chain variable region that specifically binds to CD20, CD3 or CD28; VL2 is a second immunoglobulin light chain variable region that specifically binds to CD20, CD3 or CD28; VL3 is a third immunoglobulin light chain variable region that specifically binds to CD20, CD3 or CD28; VL4 is a fourth immunoglobulin light chain variable region that specifically binds to CD20, CD3 or CD28; VH1 is a first immunoglobulin heavy chain variable region that specifically binds to CD20, CD3 or CD28; VH2 is a second immunoglobulin heavy chain variable region that specifically binds to CD20, CD3 or CD28; VH3 is a third immunoglobulin heavy chain variable region that specifically binds to CD20, CD3 or CD28; VH4 is a fourth immunoglobulin heavy chain variable region that specifically binds to CD20, CD3 or CD28; Fc is a region comprising an immunoglobulin heavy chain constant region 2 (CH2), an immunoglobulin heavy chain constant region 3 (CH3), and optionally, an immunoglobulin hinge; and L1-L8 are amino acid linkers. For example, VL1 is a first immunoglobulin light chain variable region that specifically binds to CD20; VL2 is a second immunoglobulin light chain variable region that specifically binds to CD20; VL3 is a third immunoglobulin light chain variable region that specifically binds to CD3; VL4 is a fourth immunoglobulin light chain variable region that specifically binds to CD20; VH1 is a first immunoglobulin heavy chain variable region that specifically binds to CD20; VH2 is a second immunoglobulin heavy chain variable region that specifically binds to CD20; VH3 is a third immunoglobulin heavy chain variable region that specifically binds to CD3; and VH4 is a fourth immunoglobulin heavy chain variable region that specifically binds to CD20. For example, VL1 is a first immunoglobulin light chain variable region that specifically binds to CD20; VL2 is a second immunoglobulin light chain variable region that specifically binds to CD20; VL3 is a third immunoglobulin light chain variable region that specifically binds to CD3; VL4 is a fourth immunoglobulin light chain variable region that specifically binds to CD28; VH1 is a first immunoglobulin heavy chain variable region that specifically binds to CD20; VH2 is a second immunoglobulin heavy chain variable region that specifically binds to CD20; VH3 is a third immunoglobulin heavy chain variable region that specifically binds to CD3; and VH4 is a fourth immunoglobulin heavy chain variable region that specifically binds to CD28. For example, VL1 is a first immunoglobulin light chain variable region that specifically binds to CD20; VL2 is a second immunoglobulin light chain variable region that specifically binds to CD20; VL3 is a third immunoglobulin light chain variable region that specifically binds to CD3; VL4 is a fourth immunoglobulin light chain variable region that specifically binds to CD28; VH1 is a first immunoglobulin heavy chain variable region that specifically binds to CD20; VH2 is a second immunoglobulin heavy chain variable region that specifically binds to CD20; VH3 is a third immunoglobulin heavy chain variable region that specifically binds to CD3; and VH4 is a fourth immunoglobulin heavy chain variable region that specifically binds to CD28.

In some aspects, the first polypeptide has a structure represented by VL1-L1-VL2-L2-VH2-L3-VH1-L4-Fc and the second polypeptide has a structure represented by VL3-L5-VL4-L6-VH4-L7-VH3-L8-Fc; wherein VL1 wherein VL1 is a first immunoglobulin light chain variable region that specifically binds to CD20, BCMA, CD3 or CD28; VL2 is a second immunoglobulin light chain variable region that specifically binds to CD20, BCMA, CD3 or CD28; VL3 is a third immunoglobulin light chain variable region that specifically binds to CD20, BCMA, CD3 or CD28; VL4 is a fourth immunoglobulin light chain variable region that specifically binds to CD20, BCMA, CD3 or CD28; VH1 is a first immunoglobulin heavy chain variable region that specifically binds to CD20, BCMA, CD3 or CD28; VH2 is a second immunoglobulin heavy chain variable region that specifically binds to CD20, BCMA, CD3 or CD28; VH3 is a third immunoglobulin heavy chain variable region that specifically binds to CD20, BCMA, CD3 or CD28; VH4 is a fourth immunoglobulin heavy chain variable region that specifically binds to CD20, BCMA, CD3 or CD28; Fc is a region comprising an immunoglobulin heavy chain constant region 2 (CH2), an immunoglobulin heavy chain constant region 3 (CH3), and optionally, an immunoglobulin hinge; and L1-L8 are amino acid linkers. For example, VL1 is a first immunoglobulin light chain variable region that specifically binds to CD20; VL2 is a second immunoglobulin light chain variable region that specifically binds to BCMA; VL3 is a third immunoglobulin light chain variable region that specifically binds to CD3; VL4 is a fourth immunoglobulin light chain variable region that specifically binds to CD28; VH1 is a first immunoglobulin heavy chain variable region that specifically binds to CD20; VH2 is a second immunoglobulin heavy chain variable region that specifically binds to BCMA; VH3 is a third immunoglobulin heavy chain variable region that specifically binds to CD3; and VH4 is a fourth immunoglobulin heavy chain variable region that specifically binds to CD28. For example, VL1 is a first immunoglobulin light chain variable region that specifically binds to CD20; VL2 is a second immunoglobulin light chain variable region that specifically binds to BCMA; VL3 is a third immunoglobulin light chain variable region that specifically binds to CD28; VL4 is a fourth immunoglobulin light chain variable region that specifically binds to CD3; VH1 is a first immunoglobulin heavy chain variable region that specifically binds to CD20; VH2 is a second immunoglobulin heavy chain variable region that specifically binds to BCMA; VH3 is a third immunoglobulin heavy chain variable region that specifically binds to CD28; and VH4 is a fourth immunoglobulin heavy chain variable region that specifically binds to CD3. For example, VL1 is a first immunoglobulin light chain variable region that specifically binds to BCMA; VL2 is a second immunoglobulin light chain variable region that specifically binds to CD20; VL3 is a third immunoglobulin light chain variable region that specifically binds to CD3; VL4 is a fourth immunoglobulin light chain variable region that specifically binds to CD28; VH1 is a first immunoglobulin heavy chain variable region that specifically binds to BCMA; VH2 is a second immunoglobulin heavy chain variable region that specifically binds to CD20; VH3 is a third immunoglobulin heavy chain variable region that specifically binds to CD3; and VH4 is a fourth immunoglobulin heavy chain variable region that specifically binds to CD28. For example, VL1 is a first immunoglobulin light chain variable region that specifically binds to BCMA; VL2 is a second immunoglobulin light chain variable region that specifically binds to CD20; VL3 is a third immunoglobulin light chain variable region that specifically binds to CD28; VL4 is a fourth immunoglobulin light chain variable region that specifically binds to CD3; VH1 is a first immunoglobulin heavy chain variable region that specifically binds to BCMA; VH2 is a second immunoglobulin heavy chain variable region that specifically binds to CD20; VH3 is a third immunoglobulin heavy chain variable region that specifically binds to CD28; and VH4 is a fourth immunoglobulin heavy chain variable region that specifically binds to CD3. For example, VL1 is a first immunoglobulin light chain variable region that specifically binds to CD20; VL2 is a second immunoglobulin light chain variable region that specifically binds to CD20; VL3 is a third immunoglobulin light chain variable region that specifically binds to CD28; VL4 is a fourth immunoglobulin light chain variable region that specifically binds to CD3; VH1 is a first immunoglobulin heavy chain variable region that specifically binds to CD20; VH2 is a second immunoglobulin heavy chain variable region that specifically binds to CD20; VH3 is a third immunoglobulin heavy chain variable region that specifically binds to CD28; and VH4 is a fourth immunoglobulin heavy chain variable region that specifically binds to CD3.

In some aspects, the first polypeptide has a structure represented by VL1-L1-VH2-L2-VL2-L3-VH1-L4-Fc and the second polypeptide has a structure represented by VL3-L5-VH4-L6-VL4-L7-VH3-L8-Fc; wherein VL1 is a first immunoglobulin light chain variable region that specifically binds to CD20, CD19, CD3 or CD28; VL2 is a second immunoglobulin light chain variable region that specifically binds to CD20, CD19, CD3 or CD28; VL3 is a third immunoglobulin light chain variable region that specifically binds to CD20, CD19, CD3 or CD28; VL4 is a fourth immunoglobulin light chain variable region that specifically binds to CD20, CD19, CD3 or CD28; VH1 is a first immunoglobulin heavy chain variable region that specifically binds to CD20, CD19, CD3 or CD28; VH2 is a second immunoglobulin heavy chain variable region that specifically binds to CD20, CD19, CD3 or CD28; VH3 is a third immunoglobulin heavy chain variable region that specifically binds to CD20, CD19, CD3 or CD28; VH4 is a fourth immunoglobulin heavy chain variable region that specifically binds to CD20, CD19, CD3 or CD28; Fc is a region comprising an immunoglobulin heavy chain constant region 2 (CH2), an immunoglobulin heavy chain constant region 3 (CH3), and optionally, an immunoglobulin hinge; and L1-L8 are amino acid linkers. For example, VL1 is a first immunoglobulin light chain variable region that specifically binds to CD19; VL2 is a second immunoglobulin light chain variable region that specifically binds to CD20; VL3 is a third immunoglobulin light chain variable region that specifically binds to CD3; VL4 is a fourth immunoglobulin light chain variable region that specifically binds to CD28; VH1 is a first immunoglobulin heavy chain variable region that specifically binds to CD19; VH2 is a second immunoglobulin heavy chain variable region that specifically binds to CD20; VH3 is a third immunoglobulin heavy chain variable region that specifically binds to CD3; and VH4 is a fourth immunoglobulin heavy chain variable region that specifically binds to CD28. In some aspects, the antigen binding polypeptide complex comprises amino acid sequences having at least 80%, at least 85%, at least 90%, at least 95%, or 100% identity to SEQ ID NOs:742 and 760. In some aspects, the antigen binding polypeptide complex comprises amino acid sequences encoded by polynucleotides having at least 80%, at least 85%, at least 90%, at least 95%, or 100% identity to SEQ ID NOs:743 and 761.

In some aspects, the first polypeptide has a structure represented by VH1-L1-VL2-L2-VH2-L3-VL1-L4-Fc and the second polypeptide has a structure represented by VH3-L5-VL4-L6-VH4-L7-VL3-L8-Fc; wherein VL1 is a first immunoglobulin light chain variable region that specifically binds to CD20, CD19, CD3 or CD28; VL2 is a second immunoglobulin light chain variable region that specifically binds to CD20, CD19, CD3 or CD28; VL3 is a third immunoglobulin light chain variable region that specifically binds to CD20, CD19, CD3 or CD28; VL4 is a fourth immunoglobulin light chain variable region that specifically binds to CD20, CD19, CD3 or CD28; VH1 is a first immunoglobulin heavy chain variable region that specifically binds to CD20, CD19, CD3 or CD28; VH2 is a second immunoglobulin heavy chain variable region that specifically binds to CD20, CD19, CD3 or CD28; VH3 is a third immunoglobulin heavy chain variable region that specifically binds to CD20, CD19, CD3 or CD28; VH4 is a fourth immunoglobulin heavy chain variable region that specifically binds to CD20, CD19, CD3 or CD28; Fe is a region comprising an immunoglobulin heavy chain constant region 2 (CH2), an immunoglobulin heavy chain constant region 3 (CH3), and optionally, an immunoglobulin hinge; and L1-L8 are amino acid linkers. For example, VL1 is a first immunoglobulin light chain variable region that specifically binds to CD19; VL2 is a second immunoglobulin light chain variable region that specifically binds to CD20; VL3 is a third immunoglobulin light chain variable region that specifically binds to CD3; VL4 is a fourth immunoglobulin light chain variable region that specifically binds to CD28; VH1 is a first immunoglobulin heavy chain variable region that specifically binds to CD19; VH2 is a second immunoglobulin heavy chain variable region that specifically binds to CD20; VH3 is a third immunoglobulin heavy chain variable region that specifically binds to CD3; and VH4 is a fourth immunoglobulin heavy chain variable region that specifically binds to CD28. In some aspects, the antigen binding polypeptide complex comprises amino acid sequences having at least 80%, at least 85%, at least 90%, at least 95%, or 100% identity to SEQ ID NOs:778 and 796. In some aspects, the antigen binding polypeptide complex comprises amino acid sequences encoded by polynucleotides having at least 80%, at least 85%, at least 90%, at least 95%, or 100% identity to SEQ ID NOs:779 and 797.

In some aspects, the antigen binding polypeptide complex comprises amino acid sequences having at least 80%, at least 85%, at least 90%, at least 95%, or 100% identity to SEQ ID NOs:206 and 513. In some aspects, the antigen binding polypeptide complex comprises amino acid sequences encoded by polynucleotides having at least 80%, at least 85%, at least 90%, at least 95%, or 100% identity to SEQ ID NOs:207 and 514. In some aspects, the antigen binding polypeptide complex comprises amino acid sequences having at least 80%, at least 85%, at least 90%, at least 95%, or 100% identity to SEQ ID NOs:204 and 511. In some aspects, the antigen binding polypeptide complex comprises amino acid sequences encoded by polynucleotides having at least 80%, at least 85%, at least 90%, at least 95%, or 100% identity to SEQ ID NOs:205 and 512. In some aspects, the antigen binding polypeptide complex comprises amino acid sequences having at least 80%, at least 85%, at least 90%, at least 95%, or 100% identity to SEQ ID NOs:274 and 513. In some aspects, the antigen binding polypeptide complex comprises amino acid sequences encoded by polynucleotides having at least 80%, at least 85%, at least 90%, at least 95%, or 100% identity to SEQ ID NOs:275 and 514. In some aspects, the antigen binding polypeptide complex comprises amino acid sequences having at least 80%, at least 85%, at least 90%, at least 95%, or 100% identity to SEQ ID NOs:228 and 511. In some aspects, the antigen binding polypeptide complex comprises amino acid sequences encoded by polynucleotides having at least 80%, at least 85%, at least 90%, at least 95%, or 100% identity to SEQ ID NOs:229 and 512. In some aspects, the antigen binding polypeptide complex comprises amino acid sequences having at least 80%, at least 85%, at least 90%, at least 95%, or 100% identity to SEQ ID NOs:206, 160 and 162. In some aspects, the antigen binding polypeptide complex comprises amino acid sequences encoded by polynucleotides having at least 80%, at least 85%, at least 90%, at least 95%, or 100% identity to SEQ ID NOs:207, 161 and 163. In some aspects, the antigen binding polypeptide complex comprises amino acid sequences having at least 80%, at least 85%, at least 90%, at least 95%, or 100% identity to SEQ ID NOs:206, 160 and 174. In some aspects, the antigen binding polypeptide complex comprises amino acid sequences encoded by polynucleotides having at least 80%, at least 85%, at least 90%, at least 95%, or 100% identity to SEQ ID NOs:207, 161 and 175. In some aspects, the antigen binding polypeptide complex comprises amino acid sequences having at least 80%, at least 85%, at least 90%, at least 95%, or 100% identity to SEQ ID NOs:206, 162 and 176. In some aspects, the antigen binding polypeptide complex comprises amino acid sequences encoded by polynucleotides having at least 80%, at least 85%, at least 90%, at least 95%, or 100% identity to SEQ ID NOs:207, 163 and 177. In some aspects, the antigen binding polypeptide complex comprises amino acid sequences having at least 80%, at least 85%, at least 90%, at least 95%, or 100% identity to SEQ ID NOs:204, 160 and 162. In some aspects, the antigen binding polypeptide complex comprises amino acid sequences encoded by polynucleotides having at least 80%, at least 85%, at least 90%, at least 95%, or 100% identity to SEQ ID NOs:205, 161 and 163. In some aspects, the antigen binding polypeptide complex comprises amino acid sequences having at least 80%, at least 85%, at least 90%, at least 95%, or 100% identity to SEQ ID NOs:204, 160 and 174. In some aspects, the antigen binding polypeptide complex comprises amino acid sequences encoded by polynucleotides having at least 80%, at least 85%, at least 90%, at least 95%, or 100% identity to SEQ ID NOs:205, 161 and 175. In some aspects, the antigen binding polypeptide complex comprises amino acid sequences having at least 80%, at least 85%, at least 90%, at least 95%, or 100% identity to SEQ ID NOs:204, 162 and 176. In some aspects, the antigen binding polypeptide complex comprises amino acid sequences encoded by polynucleotides having at least 80%, at least 85%, at least 90%, at least 95%, or 100% identity to SEQ ID NOs:205, 163 and 177.

In some aspects, the invention is directed to an antigen binding polypeptide complex comprising a first polypeptide and a second polypeptide; wherein the first polypeptide has a structure represented by VL1-L1-VL2-L2-VH2-L3-VH1-L4-Fc; VL1-L1-VH2-L2-VL2-L3-VH1-L4-Fc; VH1-L5-VH2-L6-VL2-L7-VL1-L8-Fc; or VH1-L5-VL2-L6-VH2-L7-VL1-L8-Fc; wherein the second polypeptide has a structure represented by VL3-L9-VL4-L10-VH4-L11-VH3-L12-Fc; VL3-L9-VH4-L10-VL4-L11-VH3-L12-Fc; VH3-L13-VH4-L14-VL4-L15-VL3-L16-Fc; or VH3-L13-VL4-L14-VH4-L15-VL3-L16-Fc; wherein VL1 is a first immunoglobulin light chain variable region that specifically binds to a tumor-associated antigen; VL2 is a second immunoglobulin light chain variable region that specifically binds to a tumor-associated antigen; VL3 is a third immunoglobulin light chain variable region that specifically binds to a tumor-associated antigen; VL4 is a fourth immunoglobulin light chain variable region that specifically binds to CD3; VH1 is a first immunoglobulin heavy chain variable region that specifically binds to a tumor-associated antigen; VH2 is a second immunoglobulin heavy chain variable region that specifically binds to a tumor-associated antigen; VH3 is a third immunoglobulin heavy chain variable region that specifically binds to a tumor-associated antigen; VH4 is a fourth immunoglobulin heavy chain variable region that specifically binds to CD3; Fe is a region comprising an immunoglobulin heavy chain constant region 2 (CH2), an immunoglobulin heavy chain constant region 3 (CH3), and optionally, an immunoglobulin hinge; and L1-L16 are amino acid linkers. In some aspects, the first polypeptide has a structure represented by VL1-L1-VL2-L2-VH2-L3-VH1-L4-Fc; VL1-L1-VH2-L2-VL2-L3-VH1-L4-Fc; VH1-L5-VH2-L6-VL2-L7-VL1-L8-Fc; or VH1-L5-VL2-L6-VH2-L7-VL1-L8-Fc and the second polypeptide has a structure represented by VL3-L9-VL4-L10-VH4-L11-VH3-L12-Fc. In some aspects, the first polypeptide has a structure represented by VL1-L1-VL2-L2-VH2-L3-VH1-L4-Fc; or VL1-L1-VH2-L2-VL2-L3-VH1-L4-Fc or VH1-L5-VH2-L6-VL2-L7-VL1-L8-Fc or VH1-L5-VL2-L6-VH2-L7-VL1-L8-Fe and the second polypeptide has a structure represented by VL3-L9-VH4-L10-VL4-L11-VH3-L12-Fc. In some aspects, the first polypeptide has a structure represented by VL1-L1-VL2-L2-VH2-L3-VH1-L4-Fc; or VL1-L1-VH2-L2-VL2-L3-VH1-L4-Fc or VH1-L5-VH2-L6-VL2-L7-VL1-L8-Fc or VH1-L5-VL2-L6-VH2-L7-VL1-L8-Fc and the second polypeptide has a structure represented by VH3-L13-VH4-L14-VL4-L15-VL3-L16-Fc. In some aspects, the first polypeptide has a structure represented by VL1-L1-VL2-L2-VH2-L3-VH1-L4-Fc; or VL1-L1-VH2-L2-VL2-L3-VH1-L4-Fc or VH1-L5-VH2-L6-VL2-L7-VL1-L8-Fc or VH1-L5-VL2-L6-VH2-L7-VL1-L8-Fe and the second polypeptide has a structure represented by VH3-L13-VL4-L14-VH4-L15-VL3-L16-Fc. In some aspects, the first polypeptide has a structure represented by VL1-L1-VL2-L2-VH2-L3-VH1-L4-Fc or VH1-L5-VH2-L6-VL2-L7-VL1-L8-Fc; and the second polypeptide has a structure represented by VL3-L9-VL4-L10-VH4-L11-VH3-L12-Fc or VH3-L13-VH4-L14-VL4-L15-VL3-L16-Fc. In some aspects, the first polypeptide has a structure represented by VL1-L1-VL2-L2-VH2-L3-VH1-L4-Fc and the second polypeptide has a structure represented by VL3-L9-VL4-L10-VH4-L11-VH3-L12-Fc. In some aspects, the first polypeptide has a structure represented by VL1-L1-VL2-L2-VH2-L3-VH1-L4-Fc and the second polypeptide has a structure represented by VH3-L13-VH4-L14-VL4-L15-VL3-L16-Fc. In some aspects, the first polypeptide has a structure represented by VH1-L5-VH2-L6-VL2-L7-VL1-L8-Fc and the second polypeptide has a structure represented by VL3-L9-VL4-L10-VH4-L11-VH3-L12-Fc. In some aspects, the first polypeptide has a structure represented by VH1-L5-VH2-L6-VL2-L7-VL1-L8-Fc and the second polypeptide has a structure represented by VH3-L13-VH4-L14-VL4-L15-VL3-L16-Fc. In some aspects, the invention is directed to an antigen binding polypeptide complex comprising a first polypeptide and a second polypeptide; wherein the first polypeptide has a structure represented by VL1-L1-VL2-L2-VH2-L3-VH1-L4-CH1-L5-Fc; VL1-L1-VH2-L2-VL2-L3-VH1-L4-CH1-L5-Fc; VH1-L6-VH2-L7-VL2-L8-VL1-L9-CH1-L10-Fc; VH1-L6-VL2-L7-VH2-L8-VL1-L9-CH1-L10-Fc; VL1-L11-VL2-L12-VH2-L13-VH1-L14-CL-L15-Fc; VL1-L11-VH2-L12-VL2-L13-VH1-L14-CL-L15-Fc; VH1-L16-VH2-L17-VL2-L18-VL1-L19-CL-L20-Fc; VH1-L16-VL2-L17-VH2-L18-VL1-L19-CL-L20-Fc; VL1-L21-VL2-L22-VH2-L23-VH1-L24-CH1-L25-CL-L26-Fc; VL1-L21-VH2-L22-VL2-L23-VH1-L24-CH1-L25-CL-L26-Fc; VH1-L27-VH2-L28-VL2-L29-VL1-L30-CH1-L31-CL-L32-Fc; VH1-L27-VL2-L28-VH2-L29-VL1-L30-CH1-L31-CL-L32-Fc; VL1-L33-VL2-L34-VH2-L35-VH1-L36-CL-L37-CH1-L38-Fc; VL1-L33-VH2-L34-VL2-L35-VH1-L36-CL-L37-CH1-L38-Fc; VH1-L39-VH2-L40-VL2-L41-VL1-L42-CL-L43-CH1-L44-Fc; or VH1-L39-VL2-L40-VH2-L41-VL1-L42-CL-L43-CH1-L44-Fc; wherein the second polypeptide has a structure represented by VL3-L45-VL4-L46-VH4-L47-VH3-L48-CH1-L49-Fc; VL3-L45-VH4-L46-VL4-L47-VH3-L48-CH1-L49-Fc; VH3-L50-VH4-L51-VL4-L52-VL3-L53-CH1-L54-Fc; VH3-L50-VL4-L51-VH4-L52-VL3-L53-CH1-L54-Fc; VL3-L55-VL4-L56-VH4-L57-VH3-L58-CL-L59-Fc; VL3-L55-VH4-L56-VL4-L57-VH3-L58-CL-L59-Fc; VH3-L60-VH4-L61-VL4-L62-VL3-L63-CL-L64-Fc; VH3-L60-VL4-L61-VH4-L62-VL3-L63-CL-L64-Fc; VL3-L65-VL4-L66-VH4-L67-VH3-L68-CH1-L69-CL-L70-Fc; VL3-L65-VH4-L66-VL4-L67-VH3-L68-CH1-L69-CL-L70-Fc; VH3-L71-VH4-L72-VL4-L73-VL3-L74-CH1-L75-CL-L76-Fc; VH3-L71-VL4-L72-VH4-L73-VL3-L74-CH1-L75-CL-L76-Fc; VL3-L77-VL4-L78-VH4-L79-VH3-L80-CL-L81-CH1-L82-Fc; VL3-L77-VH4-L78-VL4-L79-VH3-L80-CL-L81-CH1-L82-Fc; VH3-L83-VH4-L84-VL4-L85-VL3-L86-CL-L87-CH1-L88-Fc; or VH3-L83-VL4-L84-VH4-L85-VL3-L86-CL-L87-CH1-L88-Fc; wherein VL1 is a first immunoglobulin light chain variable region that specifically binds to a tumor-associated antigen; VL2 is a second immunoglobulin light chain variable region that specifically binds to a tumor-associated antigen; VL3 is a third immunoglobulin light chain variable region that specifically binds to CD3; VL4 is a fourth immunoglobulin light chain variable region that specifically binds to a tumor-associated antigen; VH1 is a first immunoglobulin heavy chain variable region that specifically binds to a tumor-associated antigen; VH2 is a second immunoglobulin heavy chain variable region that specifically binds to a tumor-associated antigen; VH3 is a third immunoglobulin heavy chain variable region that specifically binds to CD3; VH4 is a fourth immunoglobulin heavy chain variable region that specifically binds to a tumor-associated antigen; Fc is a region comprising an immunoglobulin heavy chain constant region 2 (CH2), an immunoglobulin heavy chain constant region 3 (CH3), and optionally, an immunoglobulin hinge; CH1 is an immunoglobulin heavy chain constant region 1; CL is an immunoglobulin light chain constant region; and L1-L88 are amino acid linkers. In some aspects, the first polypeptide has a structure represented by VL1-L1-VL2-L2-VH2-L3-VH1-L4-CH1-L5-Fc; VL1-L1-VH2-L2-VL2-L3-VH1-L4-CH1-L5-Fc; VH1-L6-VH2-L7-VL2-L8-VL1-L9-CH1-L10-Fc; VH1-L6-VL2-L7-VH2-L8-VL1-L9-CH1-L10-Fc; VL1-L11-VL2-L12-VH2-L13-VH1-L14-CL-L15-Fc; VL1-L11-VH2-L12-VL2-L13-VH1-L14-CL-L15-Fc; VH1-L16-VH2-L17-VL2-L18-VL1-L19-CL-L20-Fc; VH1-L16-VL2-L17-VH2-L18-VL1-L19-CL-L20-Fc; VL1-L21-VL2-L22-VH2-L23-VH1-L24-CH1-L25-CL-L26-Fc; VL1-L21-VH2-L22-VL2-L23-VH1-L24-CH1-L25-CL-L26-Fc; VH1-L27-VH2-L28-VL2-L29-VL1-L30-CH1-L31-CL-L32-Fc; VH1-L27-VL2-L28-VH2-L29-VL1-L30-CH1-L31-CL-L32-Fc; VL1-L33-VL2-L34-VH2-L35-VH1-L36-CL-L37-CH1-L38-Fc; VL1-L33-VH2-L34-VL2-L35-VH1-L36-CL-L37-CH1-L38-Fc; VH1-L39-VH2-L40-VL2-L41-VL1-L42-CL-L43-CH1-L44-Fc; or VH1-L39-VL2-L40-VH2-L41-VL1-L42-CL-L43-CH1-L44-Fc; and the second polypeptide has a structure represented by VL3-L45-VL4-L46-VH4-L47-VH3-L48-CH1-L49-Fc. In some aspects, the first polypeptide has a structure represented by VL1-L1-VL2-L2-VH2-L3-VH1-L4-CH1-L5-Fc; VL1-L1-VH2-L2-VL2-L3-VH1-L4-CH1-L5-Fc; VH1-L6-VH2-L7-VL2-L8-VL1-L9-CH1-L10-Fc; VH1-L6-VL2-L7-VH2-L8-VL1-L9-CH1-L10-Fc; VL1-L11-VL2-L12-VH2-L13-VH1-L14-CL-L15-Fc; VL1-L11-VH2-L12-VL2-L13-VH1-L14-CL-L15-Fc; VH1-L16-VH2-L17-VL2-L18-VL1-L19-CL-L20-Fc; VH1-L16-VL2-L17-VH2-L18-VL1-L19-CL-L20-Fc; VL1-L21-VL2-L22-VH2-L23-VH1-L24-CH1-L25-CL-L26-Fc; VL1-L21-VH2-L22-VL2-L23-VH1-L24-CH1-L25-CL-L26-Fc; VH1-L27-VH2-L28-VL2-L29-VL1-L30-CH1-L31-CL-L32-Fc; VH1-L27-VL2-L28-VH2-L29-VL1-L30-CH1-L31-CL-L32-Fc; VL1-L33-VL2-L34-VH2-L35-VH1-L36-CL-L37-CH1-L38-Fc; VL1-L33-VH2-L34-VL2-L35-VH1-L36-CL-L37-CH1-L38-Fc; VH1-L39-VH2-L40-VL2-L41-VL1-L42-CL-L43-CH1-L44-Fc; or VH1-L39-VL2-L40-VH2-L41-VL1-L42-CL-L43-CH1-L44-Fc; and the second polypeptide has a structure represented by VL3-L45-VH4-L46-VL4-L47-VH3-L48-CH1-L49-Fc. In some aspects, the first polypeptide has a structure represented by VL1-L1-VL2-L2-VH2-L3-VH1-L4-CH1-L5-Fc; VL1-L1-VH2-L2-VL2-L3-VH1-L4-CH1-L5-Fc; VH1-L6-VH2-L7-VL2-L8-VL1-L9-CH1-L10-Fc; VH1-L6-VL2-L7-VH2-L8-VL1-L9-CH1-L10-Fc; VL1-L11-VL2-L12-VH2-L13-VH1-L14-CL-L15-Fc; VL1-L11-VH2-L12-VL2-L13-VH1-L14-CL-L15-Fc; VH1-L16-VH2-L17-VL2-L18-VL1-L19-CL-L20-Fc; VH1-L16-VL2-L17-VH2-L18-VL1-L19-CL-L20-Fc; VL1-L21-VL2-L22-VH2-L23-VH1-L24-CH1-L25-CL-L26-Fc; VL1-L21-VH2-L22-VL2-L23-VH1-L24-CH1-L25-CL-L26-Fc; VH1-L27-VH2-L28-VL2-L29-VL1-L30-CH1-L31-CL-L32-Fc; VH1-L27-VL2-L28-VH2-L29-VL1-L30-CH1-L31-CL-L32-Fc; VL1-L33-VL2-L34-VH2-L35-VH1-L36-CL-L37-CH1-L38-Fc; VL1-L33-VH2-L34-VL2-L35-VH1-L36-CL-L37-CH1-L38-Fc; VH1-L39-VH2-L40-VL2-L41-VL1-L42-CL-L43-CH1-L44-Fc; or VH1-L39-VL2-L40-VH2-L41-VL1-L42-CL-L43-CH1-L44-Fc; and the second polypeptide has a structure represented by VH3-L50-VH4-L51-VL4-L52-VL3-L53-CH1-L54-Fc. In some aspects, the first polypeptide has a structure represented by VL1-L1-VL2-L2-VH2-L3-VH1-L4-CH1-L5-Fc; VL1-L1-VH2-L2-VL2-L3-VH1-L4-CH1-L5-Fc; VH1-L6-VH2-L7-VL2-L8-VL1-L9-CH1-L10-Fc; VH1-L6-VL2-L7-VH2-L8-VL1-L9-CH1-L10-Fc; VL1-L11-VL2-L12-VH2-L13-VH1-L14-CL-L15-Fc; VL1-L11-VH2-L12-VL2-L13-VH1-L14-CL-L15-Fc; VH1-L16-VH2-L17-VL2-L18-VL1-L19-CL-L20-Fc; VH1-L16-VL2-L17-VH2-L18-VL1-L19-CL-L20-Fc; VL1-L21-VL2-L22-VH2-L23-VH1-L24-CH1-L25-CL-L26-Fc; VL1-L21-VH2-L22-VL2-L23-VH1-L24-CH1-L25-CL-L26-Fc; VH1-L27-VH2-L28-VL2-L29-VL1-L30-CH1-L31-CL-L32-Fc; VH1-L27-VL2-L28-VH2-L29-VL1-L30-CH1-L31-CL-L32-Fc; VL1-L33-VL2-L34-VH2-L35-VH1-L36-CL-L37-CH1-L38-Fc; VL1-L33-VH2-L34-VL2-L35-VH1-L36-CL-L37-CH1-L38-Fc; VH1-L39-VH2-L40-VL2-L41-VL1-L42-CL-L43-CH1-L44-Fc; or VH1-L39-VL2-L40-VH2-L41-VL1-L42-CL-L43-CH1-L44-Fc; and the second polypeptide has a structure represented by VH3-L50-VL4-L51-VH4-L52-VL3-L53-CH1-L54-Fc. In some aspects, the first polypeptide has a structure represented by VL1-L1-VL2-L2-VH2-L3-VH1-L4-CH1-L5-Fc; VL1-L1-VH2-L2-VL2-L3-VH1-L4-CH1-L5-Fc; VH1-L6-VH2-L7-VL2-L8-VL1-L9-CH1-L10-Fc; VH1-L6-VL2-L7-VH2-L8-VL1-L9-CH1-L10-Fc; VL1-L11-VL2-L12-VH2-L13-VH1-L14-CL-L15-Fc; VL1-L11-VH2-L12-VL2-L13-VH1-L14-CL-L15-Fc; VH1-L16-VH2-L17-VL2-L18-VL1-L19-CL-L20-Fc; VH1-L16-VL2-L17-VH2-L18-VL1-L19-CL-L20-Fc; VL1-L21-VL2-L22-VH2-L23-VH1-L24-CH1-L25-CL-L26-Fc; VL1-L21-VH2-L22-VL2-L23-VH1-L24-CH1-L25-CL-L26-Fc; VH1-L27-VH2-L28-VL2-L29-VL1-L30-CH1-L31-CL-L32-Fc; VH1-L27-VL2-L28-VH2-L29-VL1-L30-CH1-L31-CL-L32-Fc; VL1-L33-VL2-L34-VH2-L35-VH1-L36-CL-L37-CH1-L38-Fc; VL1-L33-VH2-L34-VL2-L35-VH1-L36-CL-L37-CH1-L38-Fc; VH1-L39-VH2-L40-VL2-L41-VL1-L42-CL-L43-CH1-L44-Fc; or VH1-L39-VL2-L40-VH2-L41-L1-L42-CL-L43-CH1-L44-Fc; and the second polypeptide has a structure represented by VL3-L55-VL4-L56-VH4-L57-VH3-L58-CL-L59-Fc. In some aspects, the first polypeptide has a structure represented by VL1-L1-VL2-L2-VH2-L3-VH1-L4-CH1-L5-Fc; VL1-L1-VH2-L2-VL2-L3-VH1-L4-CH1-L5-Fc; VH1-L6-VH2-L7-VL2-L8-VL1-L9-CH1-L10-Fc; VH1-L6-VL2-L7-VH2-L8-VL1-L9-CH1-L10-Fc; VL1-L11-VL2-L12-VH2-L13-VH1-L14-CL-L15-Fc; VL1-L11-VH2-L12-VL2-L13-VH1-L14-CL-L15-Fc; VH1-L16-VH2-L17-VL2-L18-VL1-L19-CL-L20-Fc; VH1-L16-VL2-L17-VH2-L18-VL1-L19-CL-L20-Fc; VL1-L21-VL2-L22-VH2-L23-VH1-L24-CH1-L25-CL-L26-Fc; VL1-L21-VH2-L22-VL2-L23-VH1-L24-CH1-L25-CL-L26-Fc; VH1-L27-VH2-L28-VL2-L29-VL1-L30-CH1-L31-CL-L32-Fc; VH1-L27-VL2-L28-VH2-L29-VL1-L30-CH1-L31-CL-L32-Fc; VL1-L33-VL2-L34-VH2-L35-VH1-L36-CL-L37-CH1-L38-Fc; VL1-L33-VH2-L34-VL2-L35-VH1-L36-CL-L37-CH1-L38-Fc; VH1-L39-VH2-L40-VL2-L41-VL1-L42-CL-L43-CH1-L44-Fc; or VH1-L39-VL2-L40-VH2-L41-VL1-L42-CL-L43-CH1-L44-Fc; and the second polypeptide has a structure represented by VL3-L55-VH4-L56-VL4-L57-VH3-L58-CL-L59-Fc. In some aspects, the first polypeptide has a structure represented by VL1-L1-VL2-L2-VH2-L3-VH1-L4-CH1-L5-Fc; VL1-L1-VH2-L2-VL2-L3-VH1-L4-CH1-L5-Fc; VH1-L6-VH2-L7-VL2-L8-VL1-L9-CH1-L10-Fc; VH1-L6-VL2-L7-VH2-L8-VL1-L9-CH1-L10-Fc; VL1-L11-VL2-L12-VH2-L13-VH1-L14-CL-L15-Fc; VL1-L11-VH2-L12-VL2-L13-VH1-L14-CL-L15-Fc; VH1-L16-VH2-L17-VL2-L18-VL1-L19-CL-L20-Fc; VH1-L16-VL2-L17-VH2-L18-VL1-L19-CL-L20-Fc; VL1-L21-VL2-L22-VH2-L23-VH1-L24-CH1-L25-CL-L26-Fc; VL1-L21-VH2-L22-VL2-L23-VH1-L24-CH1-L25-CL-L26-Fc; VH1-L27-VH2-L28-VL2-L29-VL1-L30-CH1-L31-CL-L32-Fc; VH1-L27-VL2-L28-VH2-L29-VL1-L30-CH1-L31-CL-L32-Fc; VL1-L33-VL2-L34-VH2-L35-VH1-L36-CL-L37-CH1-L38-Fc; VL1-L33-VH2-L34-VL2-L35-VH1-L36-CL-L37-CH1-L38-Fc; VH1-L39-VH2-L40-VL2-L41-VL1-L42-CL-L43-CH1-L44-Fc; or VH1-L39-VL2-L40-VH2-L41-VL1-L42-CL-L43-CH1-L44-Fc; and the second polypeptide has a structure represented by VH3-L60-VH4-L61-VL4-L62-VL3-L63-CL-L64-Fc. In some aspects, the first polypeptide has a structure represented by VL1-L1-VL2-L2-VH2-L3-VH1-L4-CH1-L5-Fc; VL1-L1-VH2-L2-VL2-L3-VH1-L4-CH1-L5-Fc; VH1-L6-VH2-L7-VL2-L8-VL1-L9-CH1-L10-Fc; VH1-L6-VL2-L7-VH2-L8-VL1-L9-CH1-L10-Fc; VL1-L11-VL2-L12-VH2-L13-VH1-L14-CL-L15-Fc; VL1-L11-VH2-L12-VL2-L13-VH1-L14-CL-L15-Fc; VH1-L16-VH2-L17-VL2-L18-VL1-L19-CL-L20-Fc; VH1-L16-VL2-L17-VH2-L18-VL1-L19-CL-L20-Fc; VL1-L21-VL2-L22-VH2-L23-VH1-L24-CH1-L25-CL-L26-Fc; VL1-L21-VH2-L22-VL2-L23-VH1-L24-CH1-L25-CL-L26-Fc; VH1-L27-VH2-L28-VL2-L29-VL1-L30-CH1-L31-CL-L32-Fc; VH1-L27-VL2-L28-VH2-L29-VL1-L30-CH1-L31-CL-L32-Fc; VL1-L33-VL2-L34-VH2-L35-VH1-L36-CL-L37-CH1-L38-Fc; VL1-L33-VH2-L34-VL2-L35-VH1-L36-CL-L37-CH1-L38-Fc; VH1-L39-VH2-L40-VL2-L41-VL1-L42-CL-L43-CH1-L44-Fc; or VH1-L39-VL2-L40-VH2-L41-VL1-L42-CL-L43-CH1-L44-Fc; and the second polypeptide has a structure represented by VH3-L60-VL4-L61-VH4-L62-VL3-L63-CL-L64-Fc. In some aspects, the first polypeptide has a structure represented by VL1-L1-VL2-L2-VH2-L3-VH1-L4-CH1-L5-Fc; VL1-L1-VH2-L2-VL2-L3-VH1-L4-CH1-L5-Fc; VH1-L6-VH2-L7-VL2-L8-VL1-L9-CH1-L10-Fc; VH1-L6-VL2-L7-VH2-L8-VL1-L9-CH1-L10-Fc; VL1-L11-VL2-L12-VH2-L13-VH1-L14-CL-L15-Fc; VL1-L11-VH2-L12-VL2-L13-VH1-L14-CL-L15-Fc; VH1-L16-VH2-L17-VL2-L18-VL1-L19-CL-L20-Fc; VH1-L16-VL2-L17-VH2-L18-VL1-L19-CL-L20-Fc; VL1-L21-VL2-L22-VH2-L23-VH1-L24-CH1-L25-CL-L26-Fc; VL1-L21-VH2-L22-VL2-L23-VH1-L24-CH1-L25-CL-L26-Fc; VH1-L27-VH2-L28-VL2-L29-VL1-L30-CH1-L31-CL-L32-Fc; VH1-L27-VL2-L28-VH2-L29-VL1-L30-CH1-L31-CL-L32-Fc; VL1-L33-VL2-L34-VH2-L35-VH1-L36-CL-L37-CH1-L38-Fc; VL1-L33-VH2-L34-VL2-L35-VH1-L36-CL-L37-CH1-L38-Fc; VH1-L39-VH2-L40-VL2-L41-VL1-L42-CL-L43-CH1-L44-Fc; or VH1-L39-VL2-L40-VH2-L41-VL1-L42-CL-L43-CH1-L44-Fc; and the second polypeptide has a structure represented by VL3-L65-VL4-L66-VH4-L67-VH3-L68-CH1-L69-CL-L70-Fc. In some aspects, the first polypeptide has a structure represented by VL1-L1-VL2-L2-VH2-L3-VH1-L4-CH1-L5-Fc; VL1-L1-VH2-L2-VL2-L3-VH1-L4-CH1-L5-Fc; VH1-L6-VH2-L7-VL2-L8-VL1-L9-CH1-L10-Fc; VH1-L6-VL2-L7-VH2-L8-VL1-L9-CH1-L10-Fc; VL1-L11-VL2-L12-VH2-L13-VH1-L14-CL-L15-Fc; VL1-L11-VH2-L12-VL2-L13-VH1-L14-CL-L15-Fc; VH1-L16-VH2-L17-VL2-L18-VL1-L19-CL-L20-Fc; VH1-L16-VL2-L17-VH2-L18-VL1-L19-CL-L20-Fc; VL1-L21-VL2-L22-VH2-L23-VH1-L24-CH1-L25-CL-L26-Fc; VL1-L21-VH2-L22-VL2-L23-VH1-L24-CH1-L25-CL-L26-Fc; VH1-L27-VH2-L28-VL2-L29-VL1-L30-CH1-L31-CL-L32-Fc; VH1-L27-VL2-L28-VH2-L29-VL1-L30-CH1-L31-CL-L32-Fc; VL1-L33-VL2-L34-VH2-L35-VH1-L36-CL-L37-CH1-L38-Fc; VL1-L33-VH2-L34-VL2-L35-VH1-L36-CL-L37-CH1-L38-Fc; VH1-L39-VH2-L40-VL2-L41-VL1-L42-CL-L43-CH1-L44-Fe; or VH1-L39-VL2-L40-VH2-L41-VL1-L42-CL-L43-CH1-L44-Fc; and the second polypeptide has a structure represented by VL3-L65-VH4-L66-VL4-L67-VH3-L68-CH1-L69-CL-L70-Fc. In some aspects, the first polypeptide has a structure represented by VL1-L1-VL2-L2-VH2-L3-VH1-L4-CH1-L5-Fc; VL1-L1-VH2-L2-VL2-L3-VH1-L4-CH1-L5-Fc; VH1-L6-VH2-L7-VL2-L8-VL1-L9-CH1-L10-Fc; VH1-L6-VL2-L7-VH2-L8-VL1-L9-CH1-L10-Fc; VL1-L11-VL2-L12-VH2-L13-VH1-L14-CL-L15-Fc; VL1-L11-VH2-L12-VL2-L13-VH1-L14-CL-L15-Fc; VH1-L16-VH2-L17-VL2-L18-VL1-L19-CL-L20-Fc; VH1-L16-VL2-L17-VH2-L18-VL1-L19-CL-L20-Fc; VL1-L21-VL2-L22-VH2-L23-VH1-L24-CH1-L25-CL-L26-Fc; VL1-L21-VH2-L22-VL2-L23-VH1-L24-CH1-L25-CL-L26-Fc; VH1-L27-VH2-L28-VL2-L29-VL1-L30-CH1-L31-CL-L32-Fc; VH1-L27-VL2-L28-VH2-L29-VL1-L30-CH1-L31-CL-L32-Fc; VL1-L33-VL2-L34-VH2-L35-VH1-L36-CL-L37-CH1-L38-Fc; VL1-L33-VH2-L34-VL2-L35-VH1-L36-CL-L37-CH1-L38-Fc; VH1-L39-VH2-L40-VL2-L41-VL1-L42-CL-L43-CH1-L44-Fc; or VH1-L39-VL2-L40-VH2-L41-VL1-L42-CL-L43-CH1-L44-Fc; and the second polypeptide has a structure represented by VH3-L71-VH4-L72-VL4-L73-VL3-L74-CH1-L75-CL-L76-Fc. In some aspects, the first polypeptide has a structure represented by VL1-L1-VL2-L2-VH2-L3-VH1-L4-CH1-L5-Fc; VL1-L1-VH2-L2-VL2-L3-VH1-L4-CH1-L5-Fc; VH1-L6-VH2-L7-VL2-L8-VL1-L9-CH1-L10-Fc; VH1-L6-VL2-L7-VH2-L8-VL1-L9-CH1-L10-Fc; VL1-L11-VL2-L12-VH2-L13-VH1-L14-CL-L15-Fc; VL1-L11-VH2-L12-VL2-L13-VH1-L14-CL-L15-Fc; VH1-L16-VH2-L17-VL2-L18-VL1-L19-CL-L20-Fc; VH1-L16-VL2-L17-VH2-L18-VL1-L19-CL-L20-Fc; VL1-L21-VL2-L22-VH2-L23-VH1-L24-CH1-L25-CL-L26-Fc; VL1-L21-VH2-L22-VL2-L23-VH1-L24-CH1-L25-CL-L26-Fc; VH1-L27-VH2-L28-VL2-L29-VL1-L30-CH1-L31-CL-L32-Fc; VH1-L27-VL2-L28-VH2-L29-VL1-L30-CH1-L31-CL-L32-Fc; VL1-L33-VL2-L34-VH2-L35-VH1-L36-CL-L37-CH1-L38-Fc; VL1-L33-VH2-L34-VL2-L35-VH1-L36-CL-L37-CH1-L38-Fc; VH1-L39-VH2-L40-VL2-L41-VL1-L42-CL-L43-CH1-L44-Fc; or VH1-L39-VL2-L40-VH2-L41-VL1-L42-CL-L43-CH1-L44-Fc; and the second polypeptide has a structure represented by VH3-L71-VL4-L72-VH4-L73-VL3-L74-CH1-L75-CL-L76-Fc. In some aspects, the first polypeptide has a structure represented by VL1-L1-VL2-L2-VH2-L3-VH1-L4-CH1-L5-Fc; VL1-L1-VH2-L2-VL2-L3-VH1-L4-CH1-L5-Fc; VH1-L6-VH2-L7-VL2-L8-VL1-L9-CH1-L10-Fc; VH1-L6-VL2-L7-VH2-L8-VL1-L9-CH1-L10-Fc; VL1-L11-VL2-L12-VH2-L13-VH1-L14-CL-L15-Fc; VL1-L11-VH2-L12-VL2-L13-VH1-L14-CL-L15-Fc; VH1-L16-VH2-L17-VL2-L18-VL1-L19-CL-L20-Fc; VH1-L16-VL2-L17-VH2-L18-VL1-L19-CL-L20-Fc; VL1-L21-VL2-L22-VH2-L23-VH1-L24-CH1-L25-CL-L26-Fc; VL1-L21-VH2-L22-VL2-L23-VH1-L24-CH1-L25-CL-L26-Fc; VH1-L27-VH2-L28-VL2-L29-VL1-L30-CH1-L31-CL-L32-Fc; VH1-L27-VL2-L28-VH2-L29-VL1-L30-CH1-L31-CL-L32-Fc; VL1-L33-VL2-L34-VH2-L35-VH1-L36-CL-L37-CH1-L38-Fc; VL1-L33-VH2-L34-VL2-L35-VH1-L36-CL-L37-CH1-L38-Fc; VH1-L39-VH2-L40-VL2-L41-VL1-L42-CL-L43-CH1-L44-Fc; or VH1-L39-VL2-L40-VH2-L41-VL1-L42-CL-L43-CH1-L44-Fc; and the second polypeptide has a structure represented by VL3-L77-VL4-L78-VH4-L79-VH3-L80-CL-L81-CH1-L82-Fc. In some aspects, the first polypeptide has a structure represented by VL1-L1-VL2-L2-VH2-L3-VH1-L4-CH1-L5-Fc; VL1-L1-VH2-L2-VL2-L3-VH1-L4-CH1-L5-Fc; VH1-L6-VH2-L7-VL2-L8-VL1-L9-CH1-L10-Fc; VH1-L6-VL2-L7-VH2-L8-VL1-L9-CH1-L10-Fc; VL1-L11-VL2-L12-VH2-L13-VH1-L14-CL-L15-Fc; VL1-L11-VH2-L12-VL2-L13-VH1-L14-CL-L15-Fc; VH1-L16-VH2-L17-VL2-L18-VL1-L19-CL-L20-Fc; VH1-L16-VL2-L17-VH2-L18-VL1-L19-CL-L20-Fc; VL1-L21-VL2-L22-VH2-L23-VH1-L24-CH1-L25-CL-L26-Fc; VL1-L21-VH2-L22-VL2-L23-VH1-L24-CH1-L25-CL-L26-Fc; VH1-L27-VH2-L28-VL2-L29-VL1-L30-CH1-L31-CL-L32-Fc; VH1-L27-VL2-L28-VH2-L29-VL1-L30-CH1-L31-CL-L32-Fc; VL1-L33-VL2-L34-VH2-L35-VH1-L36-CL-L37-CH1-L38-Fc; VL1-L33-VH2-L34-VL2-L35-VH1-L36-CL-L37-CH1-L38-Fc; VH1-L39-VH2-L40-VL2-L41-VL1-L42-CL-L43-CH1-L44-Fc; or VH1-L39-VL2-L40-VH2-L41-VL1-L42-CL-L43-CH1-L44-Fc; and the second polypeptide has a structure represented by VL3-L77-VH4-L78-VL4-L79-VH3-L80-CL-L81-CH1-L82-Fc. In some aspects, the first polypeptide has a structure represented by VL1-L1-VL2-L2-VH2-L3-VH1-L4-CH1-L5-Fc; VL1-L1-VH2-L2-VL2-L3-VH1-L4-CH1-L5-Fc; VH1-L6-VH2-L7-VL2-L8-VL1-L9-CH1-L10-Fc; VH1-L6-VL2-L7-VH2-L8-VL1-L9-CH1-L10-Fc; VL1-L11-VL2-L12-VH2-L13-VH1-L14-CL-L15-Fc; VL1-L11-VH2-L12-VL2-L13-VH1-L14-CL-L15-Fc; VH1-L16-VH2-L17-VL2-L18-VL1-L19-CL-L20-Fc; VH1-L16-VL2-L17-VH2-L18-VL1-L19-CL-L20-Fc; VL1-L21-VL2-L22-VH2-L23-VH1-L24-CH1-L25-CL-L26-Fc; VL1-L21-VH2-L22-VL2-L23-VH1-L24-CH1-L25-CL-L26-Fc; VH1-L27-VH2-L28-VL2-L29-VL1-L30-CH1-L31-CL-L32-Fc; VH1-L27-VL2-L28-VH2-L29-VL1-L30-CH1-L31-CL-L32-Fc; VL1-L33-VL2-L34-VH2-L35-VH1-L36-CL-L37-CH1-L38-Fc; VL1-L33-VH2-L34-VL2-L35-VH1-L36-CL-L37-CH1-L38-Fc; VH1-L39-VH2-L40-VL2-L41-VL1-L42-CL-L43-CH1-L44-Fc; or VH1-L39-VL2-L40-VH2-L41-VL1-L42-CL-L43-CH1-L44-Fc; and the second polypeptide has a structure represented by VH3-L83-VH4-L84-VL4-L85-VL3-L86-CL-L87-CH1-L88-Fc. In some aspects, the first polypeptide has a structure represented by VL1-L1-VL2-L2-VH2-L3-VH1-L4-CH1-L5-Fc; VL1-L1-VH2-L2-VL2-L3-VH1-L4-CH1-L5-Fc; VH1-L6-VH2-L7-VL2-L8-VL1-L9-CH1-L10-Fc; VH1-L6-VL2-L7-VH2-L8-VL1-L9-CH1-L10-Fc; VL1-L11-VL2-L12-VH2-L13-VH1-L14-CL-L15-Fc; VL1-L11-VH2-L12-VL2-L13-VH1-L14-CL-L15-Fc; VH1-L16-VH2-L17-VL2-L18-VL1-L19-CL-L20-Fc; VH1-L16-VL2-L17-VH2-L18-VL1-L19-CL-L20-Fc; VL1-L21-VL2-L22-VH2-L23-VH1-L24-CH1-L25-CL-L26-Fc; VL1-L21-VH2-L22-VL2-L23-VH1-L24-CH1-L25-CL-L26-Fc; VH1-L27-VH2-L28-VL2-L29-VL1-L30-CH1-L31-CL-L32-Fc; VH1-L27-VL2-L28-VH2-L29-VL1-L30-CH1-L31-CL-L32-Fc; VL1-L33-VL2-L34-VH2-L35-VH1-L36-CL-L37-CH1-L38-Fc; VL1-L33-VH2-L34-VL2-L35-VH1-L36-CL-L37-CH1-L38-Fc; VH1-L39-VH2-L40-VL2-L41-VL1-L42-CL-L43-CH1-L44-Fc; or VH1-L39-VL2-L40-VH2-L41-VL1-L42-CL-L43-CH1-L44-Fc; and the second polypeptide has a structure represented by VH3-L83-VL4-L84-VH4-L85-VL3-L86-CL-L87-CH1-L88-Fc. In some aspects, the first polypeptide has a structure represented by: VL1-L1-VL2-L2-VH2-L3-VH1-L4-CH1-L5-Fc; VH1-L6-VH2-L7-VL2-L8-VL1-L9-CH1-L10-Fc; VL1-L11-VL2-L12-VH2-L13-VH1-L14-CL-L15-Fc; VH1-L16-VH2-L17-VL2-L18-VL1-L19-CL-L20-Fc; VL1-L21-VL2-L22-VH2-L23-VH1-L24-CH1-L25-CL-L26-Fc; VH1-L27-VH2-L28-VL2-L29-VL1-L30-CH1-L31-CL-L32-Fc; VL1-L33-VL2-L34-VH2-L35-VH1-L36-CL-L37-CH1-L38-Fc; or VH1-L39-VH2-L40-VL2-L41-VL1-L42-CL-L43-CH1-L44-Fc; and the second polypeptide has a structure represented by: VL3-L45-VL4-L46-VH4-L47-VH3-L48-CH1-L49-Fc; VH3-L50-VH4-L51-VL4-L52-VL3-L53-CH1-L54-Fc; VL3-L55-VL4-L56-VH4-L57-VH3-L58-CL-L59-Fc; VH3-L60-VH4-L61-VL4-L62-VL3-L63-CL-L64-Fc; VL3-L65-VL4-L66-VH4-L67-VH3-L68-CH1-L69-CL-L70-Fc; VH3-L71-VH4-L72-VL4-L73-VL3-L74-CH1-L75-CL-L76-Fc; VL3-L77-VL4-L78-VH4-L79-VH3-L80-CL-L81-CH1-L82-Fc; or VH3-L83-VH4-L84-VL4-L85-VL3-L86-CL-L87-CH1-L88-Fc.

In other some aspects, the invention is directed to an antigen binding polypeptide complex comprising a first polypeptide and a second polypeptide; wherein the first polypeptide has a structure represented by VL1-L1-VL2-L2-VH2-L3-VH1-L4-Fc; or VL1-L1-VH2-L2-VL2-L3-VH1-L4-Fc; or VH1-L5-VH2-L6-VL2-L7-VL1-L8-Fc; or VH1-L5-VL2-L6-VH2-L7-VL1-L8-Fc; wherein the second polypeptide has a structure represented by VL3-L9-VL4-L10-VH4-L11-VH3-L12-Fc; or VL3-L9-VH4-L10-VL4-L11-VH3-L12-Fc; or VH3-L13-VH4-L14-VL4-L15-VL3-L16-Fc; or VH3-L13-VL4-L14-VH4-L15-VL3-L16-Fc; wherein VL1 is a first immunoglobulin light chain variable region; VL2 is a second immunoglobulin light chain variable region; VL3 is a third immunoglobulin light chain variable region; VL4 is a fourth immunoglobulin light chain variable region; VH1 is a first immunoglobulin heavy chain variable region; VH2 is a second immunoglobulin heavy chain variable region; VH3 is a third immunoglobulin heavy chain variable region; VH4 is a fourth immunoglobulin heavy chain variable region; Fc is a region comprising an immunoglobulin heavy chain constant region 2 (CH2), an immunoglobulin heavy chain constant region 3 (CH3), and optionally, an immunoglobulin hinge; and L1-L16 are amino acid linkers. In some aspects, the first polypeptide has a structure represented by VL1-L1-VL2-L2-VH2-L3-VH1-L4-Fc; VL1-L1-VH2-L2-VL2-L3-VH1-L4-Fc; VH1-L5-VH2-L6-VL2-L7-VL1-L8-Fc; or VH1-L5-VL2-L6-VH2-L7-VL1-L8-Fc and the second polypeptide has a structure represented by VL3-L9-VL4-L10-VH4-L11-VH3-L12-Fc. In some aspects, the first polypeptide has a structure represented by VL1-L1-VL2-L2-VH2-L3-VH1-L4-Fc; or VL1-L1-VH2-L2-VL2-L3-VH1-L4-Fc or VH1-L5-VH2-L6-VL2-L7-VL1-L8-Fc or VH1-L5-VL2-L6-VH2-L7-VL1-L8-Fc and the second polypeptide has a structure represented by VL3-L9-VH4-L10-VL4-L11-VH3-L12-Fc. In some aspects, the first polypeptide has a structure represented by VL1-L1-VL2-L2-VH2-L3-VH1-L4-Fc; or VL1-L1-VH2-L2-VL2-L3-VH1-L4-Fc or VH1-L5-VH2-L6-VL2-L7-VL1-L8-Fc or VH1-L5-VL2-L6-VH2-L7-VL1-L8-Fc and the second polypeptide has a structure represented by VH3-L13-VH4-L14-VL4-L15-VL3-L16-Fc. In some aspects, the first polypeptide has a structure represented by VL1-L1-VL2-L2-VH2-L3-VH1-L4-Fc; or VL1-L1-VH2-L2-VL2-L3-VH1-L4-Fc or VH1-L5-VH2-L6-VL2-L7-VL1-L8-Fc or VH1-L5-VL2-L6-VH2-L7-VL1-L8-Fc and the second polypeptide has a structure represented by VH3-L13-VL4-L14-VH4-L15-VL3-L16-Fc. In some aspects, the first polypeptide has a structure represented by VL1-L1-VL2-L2-VH2-L3-VH1-L4-Fc or VH1-L5-VH2-L6-VL2-L7-VL1-L8-Fc; and the second polypeptide has a structure represented by VL3-L9-VL4-L10-VH4-L11-VH3-L12-Fc or VH3-L13-VH4-L14-VL4-L15-VL3-L16-Fc. In some aspects, the first polypeptide has a structure represented by VL1-L1-VL2-L2-VH2-L3-VH1-L4-Fc and the second polypeptide has a structure represented by VL3-L9-VL4-L10-VH4-L11-VH3-L12-Fc. In some aspects, the first polypeptide has a structure represented by VL1-L1-VL2-L2-VH2-L3-VH1-L4-Fc and the second polypeptide has a structure represented by VH3-L13-VH4-L14-VL4-L15-VL3-L16-Fc. In some aspects, the first polypeptide has a structure represented by VH1-L5-VH2-L6-VL2-L7-VL1-L8-Fc and the second polypeptide has a structure represented by VL3-L9-VL4-L10-VH4-L11-VH3-L12-Fc. In some aspects, the first polypeptide has a structure represented by VH1-L5-VH2-L6-VL2-L7-VL1-L8-Fc and the second polypeptide has a structure represented by VH3-L13-VH4-L14-VL4-L15-VL3-L16-Fc.

In other some aspects, the invention is directed to an antigen binding polypeptide complex comprising a first polypeptide and a second polypeptide; wherein the first polypeptide has a structure represented by VL1-L1-VL2-L2-VH2-L3-VH1-L4-Fc; or VL1-L1-VH2-L2-VL2-L3-VH1-L4-Fc; or VH1-L5-VH2-L6-VL2-L7-VL1-L8-Fc; or VH1-L5-VL2-L6-VH2-VL1-L8-Fc; wherein the second polypeptide has a structure represented by VL3-L9-VL4-L10-VH4-L11-VH3-L12-Fc; or VL3-L9-VH4-L10-VL4-L11-VH3-L12-Fc; or VH3-L13-VH4-L14-VL4-L15-VL3-L16-Fc; or VH3-L13-VL4-L14-VH4-L15-VL3-L16-Fc; wherein VL1 is a first immunoglobulin light chain variable region; VL2 is a second immunoglobulin light chain variable region; VL3 is a third immunoglobulin light chain variable region; VL4 is a fourth immunoglobulin light chain variable region; VH1 is a first immunoglobulin heavy chain variable region; VH2 is a second immunoglobulin heavy chain variable region; VH3 is a third immunoglobulin heavy chain variable region; VH4 is a fourth immunoglobulin heavy chain variable region; Fe is a region comprising an immunoglobulin heavy chain constant region 2 (CH2), an immunoglobulin heavy chain constant region 3 (CH3), and optionally, an immunoglobulin hinge; and L1-L16 are amino acid linkers. In some aspects, the first polypeptide has a structure represented by VL1-L1-VL2-L2-VH2-L3-VH1-L4-Fc; VL1-L1-VH2-L2-VL2-L3-VH1-L4-Fc; VH1-L5-VH2-L6-VL2-L7-VL1-L8-Fc; or VH1-L5-VL2-L6-VH2-VL1-L8-Fc; and the second polypeptide has a structure represented by VL3-L9-VL4-L10-VH4-L11-VH3-L12-Fc. In some aspects, the first polypeptide has a structure represented by VL1-L1-VL2-L2-VH2-L3-VH1-L4-Fc; VL1-L1-VH2-L2-VL2-L3-VH1-L4-Fc; VH1-L5-VH2-L6-VL2-L7-VL1-L8-Fc; or VH1-L5-VL2-L6-VH2-VL1-L8-Fc; and the second polypeptide has a structure represented by VL3-L9-VH4-L10-VL4-L11-VH3-L12-Fc. In some aspects, the first polypeptide has a structure represented by VL1-L1-VL2-L2-VH2-L3-VH1-L4-Fc; VL1-L1-VH2-L2-VL2-L3-VH1-L4-Fc; VH1-L5-VH2-L6-VL2-L7-VL1-L8-Fc; or VH1-L5-VL2-L6-VH2-VL1-L8-Fc; and the second polypeptide has a structure represented by VH3-L13-VH4-L14-VL4-L15-VL3-L16-Fc. In some aspects, the first polypeptide has a structure represented by VL1-L1-VL2-L2-VH2-L3-VH1-L4-Fc; VL1-L1-VH2-L2-VL2-L3-VH1-L4-Fc; VH1-L5-VH2-L6-VL2-L7-VL1-L8-Fc; or VH1-L5-VL2-L6-VH2-VL1-L8-Fc; and the second polypeptide has a structure represented by VH3-L13-VL4-L14-VH4-L15-VL3-L16-Fc. In some aspects, the first polypeptide has a structure represented by VL1-L1-VL2-L2-VH2-L3-VH1-L4-Fe or VH1-L5-VH2-L6-VL2-L7-VL1-L8-Fc; and the second polypeptide has a structure represented by VL3-L9-VL4-L10-VH4-L11-VH3-L12-Fc or VH3-L13-VH4-L14-VL4-L15-VL3-L16-Fc. In some aspects, the first polypeptide has a structure represented by VL1-L1-VL2-L2-VH2-L3-VH1-L4-Fc and the second polypeptide has a structure represented by VL3-L9-VL4-L10-VH4-L11-VH3-L12-Fc. In some aspects, the first polypeptide has a structure represented by VL1-L1-VL2-L2-VH2-L3-VH1-L4-Fc and the second polypeptide has a structure represented by VH3-L13-VH4-L14-VL4-L15-VL3-L16-Fc. In some aspects, the first polypeptide has a structure represented by VH1-L5-VH2-L6-VL2-L7-VL1-L8-Fc and the second polypeptide has a structure represented by VL3-L9-VL4-L10-VH4-L11-VH3-L12-Fc. In some aspects, the first polypeptide has a structure represented by VH1-L5-VH2-L6-VL2-L7-VL1-L8-Fc and the second polypeptide has a structure represented by VH3-L13-VH4-L14-VL4-L15-VL3-L16-Fc.

In some aspects, the invention is directed to an antigen binding polypeptide complex comprising a first polypeptide and a second polypeptide; wherein the first polypeptide has a structure represented by VL1-L1-VL2-L2-VH2-L3-VH1-L4-CH1-L5-Fc; VL1-L1-VH2-L2-VL2-L3-VH1-L4-CH1-L5-Fc; VH1-L6-VH2-L7-VL2-L8-VL1-L9-CH1-L10-Fc; VH1-L6-VL2-L7-VH2-L8-VL1-L9-CH1-L10-Fc; VL1-L11-VL2-L12-VH2-L13-VH1-L14-CL-L15-Fc; VL1-L11-VH2-L12-VL2-L13-VH1-L14-CL-L15-Fc; VH1-L16-VH2-L17-VL2-L18-VL1-L19-CL-L20-Fc; VH1-L16-VL2-L17-VH2-L18-VL1-L19-CL-L20-Fc; VL1-L21-VL2-L22-VH2-L23-VH1-L24-CH1-L25-CL-L26-Fc; VL1-L21-VH2-L22-VL2-L23-VH1-L24-CH1-L25-CL-L26-Fc; VH1-L27-VH2-L28-VL2-L29-VL1-L30-CH1-L31-CL-L32-Fc; VH1-L27-VL2-L28-VH2-L29-VL1-L30-CH1-L31-CL-L32-Fc; VL1-L33-VL2-L34-VH2-L35-VH1-L36-CL-L37-CH1-L38-Fc; VL1-L33-VH2-L34-VL2-L35-VH1-L36-CL-L37-CH1-L38-Fc; VH1-L39-VH2-L40-VL2-L41-VL1-L42-CL-L43-CH1-L44-Fc; or VH1-L39-VL2-L40-VH2-L41-VL1-L42-CL-L43-CH1-L44-Fc; wherein the second polypeptide has a structure represented by VL3-L45-VL4-L46-VH4-L47-VH3-L48-CH1-L49-Fc; VL3-L45-VH4-L46-VL4-L47-VH3-L48-CH1-L49-Fc; VH3-L50-VH4-L51-VL4-L52-VL3-L53-CH1-L54-Fc; VH3-L50-VL4-L51-VH4-L52-VL3-L53-CH1-L54-Fc; VL3-L55-VL4-L56-VH4-L57-VH3-L58-CL-L59-Fc; VL3-L55-VH4-L56-VL4-L57-VH3-L58-CL-L59-Fc; VH3-L60-VH4-L61-VL4-L62-VL3-L63-CL-L64-Fc; VH3-L60-VL4-L61-VH4-L62-VL3-L63-CL-L64-Fc; VL3-L65-VL4-L66-VH4-L67-VH3-L68-CH1-L69-CL-L70-Fc; VL3-L65-VH4-L66-VL4-L67-VH3-L68-CH1-L69-CL-L70-Fc; VH3-L71-VH4-L72-VL4-L73-VL3-L74-CH1-L75-CL-L76-Fc; VH3-L71-VL4-L72-VH4-L73-VL3-L74-CH1-L75-CL-L76-Fc; VL3-L77-VL4-L78-VH4-L79-VH3-L80-CL-L81-CH1-L82-Fc; VL3-L77-VH4-L78-VL4-L79-VH3-L80-CL-L81-CH1-L82-Fc; VH3-L83-VH4-L84-VL4-L85-VL3-L86-CL-L87-CH1-L88-Fc; or VH3-L83-VL4-L84-VH4-L85-VL3-L86-CL-L87-CH1-L88-Fc; wherein VL1 is a first immunoglobulin light chain variable region that specifically binds to a tumor-associated antigen; VL2 is a second immunoglobulin light chain variable region that specifically binds to a tumor-associated antigen; VL3 is a third immunoglobulin light chain variable region that specifically binds to a tumor-associated antigen; VL4 is a fourth immunoglobulin light chain variable region that specifically binds to CD3; VH1 is a first immunoglobulin heavy chain variable region that specifically binds to a tumor-associated antigen; VH2 is a second immunoglobulin heavy chain variable region that specifically binds to a tumor-associated antigen; VH3 is a third immunoglobulin heavy chain variable region that specifically binds to a tumor-associated antigen; VH4 is a fourth immunoglobulin heavy chain variable region that specifically binds to CD3; Fc is a region comprising an immunoglobulin heavy chain constant region 2 (CH2), an immunoglobulin heavy chain constant region 3 (CH3), and optionally, an immunoglobulin hinge; CH1 is an immunoglobulin heavy chain constant region 1; CL is an immunoglobulin light chain constant region; and L1-L88 are amino acid linkers. In some aspects, the first polypeptide has a structure represented by VL1-L1-VL2-L2-VH2-L3-VH1-L4-CH1-L5-Fc; VL1-L1-VH2-L2-VL2-L3-VH1-L4-CH1-L5-Fc; VH1-L6-VH2-L7-VL2-L8-VL1-L9-CH1-L10-Fc; VH1-L6-VL2-L7-VH2-L8-VL1-L9-CH1-L10-Fc; VL1-L11-VL2-L12-VH2-L13-VH1-L14-CL-L15-Fc; VL1-L11-VH2-L12-VL2-L13-VH1-L14-CL-L15-Fc; VH1-L16-VH2-L17-VL2-L18-VL1-L19-CL-L20-Fc; VH1-L16-VL2-L17-VH2-L18-VL1-L19-CL-L20-Fc; VL1-L21-VL2-L22-VH2-L23-VH1-L24-CH1-L25-CL-L26-Fc; VL1-L21-VH2-L22-VL2-L23-VH1-L24-CH1-L25-CL-L26-Fc; VH1-L27-VH2-L28-VL2-L29-VL1-L30-CH1-L31-CL-L32-Fc; VH1-L27-VL2-L28-VH2-L29-VL1-L30-CH1-L31-CL-L32-Fc; VL1-L33-VL2-L34-VH2-L35-VH1-L36-CL-L37-CH1-L38-Fc; VL1-L33-VH2-L34-VL2-L35-VH1-L36-CL-L37-CH1-L38-Fc; VH1-L39-VH2-L40-VL2-L41-VL1-L42-CL-L43-CH1-L44-Fc; or VH1-L39-VL2-L40-VH2-L41-VL1-L42-CL-L43-CH1-L44-Fc; and the second polypeptide has a structure represented by VL3-L45-VL4-L46-VH4-L47-VH3-L48-CH1-L49-Fc. In some aspects, the first polypeptide has a structure represented by VL1-L1-VL2-L2-VH2-L3-VH1-L4-CH1-L5-Fc; VL1-L1-VH2-L2-VL2-L3-VH1-L4-CH1-L5-Fc; VH1-L6-VH2-L7-VL2-L8-VL1-L9-CH1-L10-Fc; VH1-L6-VL2-L7-VH2-L8-VL1-L9-CH1-L10-Fc; VL1-L11-VL2-L12-VH2-L13-VH1-L14-CL-L15-Fc; VL1-L11-VH2-L12-VL2-L13-VH1-L14-CL-L15-Fc; VH1-L16-VH2-L17-VL2-L18-VL1-L19-CL-L20-Fc; VH1-L16-VL2-L17-VH2-L18-VL1-L19-CL-L20-Fc; VL1-L21-VL2-L22-VH2-L23-VH1-L24-CH1-L25-CL-L26-Fc; VL1-L21-VH2-L22-VL2-L23-VH1-L24-CH1-L25-CL-L26-Fc; VH1-L27-VH2-L28-VL2-L29-VL1-L30-CH1-L31-CL-L32-Fc; VH1-L27-VL2-L28-VH2-L29-VL1-L30-CH1-L31-CL-L32-Fc; VL1-L33-VL2-L34-VH2-L35-VH1-L36-CL-L37-CH1-L38-Fc; VL1-L33-VH2-L34-VL2-L35-VH1-L36-CL-L37-CH1-L38-Fc; VH1-L39-VH2-L40-VL2-L41-VL1-L42-CL-L43-CH1-L44-Fc; or VH1-L39-VL2-L40-VH2-L41-VL1-L42-CL-L43-CH1-L44-Fc; and the second polypeptide has a structure represented by VL3-L45-VH4-L46-VL4-L47-VH3-L48-CH1-L49-Fc. In some aspects, the first polypeptide has a structure represented by VL1-L1-VL2-L2-VH2-L3-VH1-L4-CH1-L5-Fc; VL1-L1-VH2-L2-VL2-L3-VH1-L4-CH1-L5-Fc; VH1-L6-VH2-L7-VL2-L8-VL1-L9-CH1-L10-Fc; VH1-L6-VL2-L7-VH2-L8-VL1-L9-CH1-L10-Fc; VL1-L11-VL2-L12-VH2-L13-VH1-L14-CL-L15-Fc; VL1-L11-VH2-L12-VL2-L13-VH1-L14-CL-L15-Fc; VH1-L16-VH2-L17-VL2-L18-VL1-L19-CL-L20-Fc; VH1-L16-VL2-L17-VH2-L18-VL1-L19-CL-L20-Fc; VL1-L21-VL2-L22-VH2-L23-VH1-L24-CH1-L25-CL-L26-Fc; VL1-L21-VH2-L22-VL2-L23-VH1-L24-CH1-L25-CL-L26-Fc; VH1-L27-VH2-L28-VL2-L29-VL1-L30-CH1-L31-CL-L32-Fc; VH1-L27-VL2-L28-VH2-L29-VL1-L30-CH1-L31-CL-L32-Fc; VL1-L33-VL2-L34-VH2-L35-VH1-L36-CL-L37-CH1-L38-Fc; VL1-L33-VH2-L34-VL2-L35-VH1-L36-CL-L37-CH1-L38-Fc; VH1-L39-VH2-L40-VL2-L41-VL1-L42-CL-L43-CH1-L44-Fc; or VH1-L39-VL2-L40-VH2-L41-VL1-L42-CL-L43-CH1-L44-Fc; and the second polypeptide has a structure represented by VH3-L50-VH4-L51-VL4-L52-VL3-L53-CH1-L54-Fc. In some aspects, the first polypeptide has a structure represented by VL1-L1-VL2-L2-VH2-L3-VH1-L4-CH1-L5-Fc; VL1-L1-VH2-L2-VL2-L3-VH1-L4-CH1-L5-Fc; VH1-L6-VH2-L7-VL2-L8-VL1-L9-CH1-L10-Fc; VH1-L6-VL2-L7-VH2-L8-VL1-L9-CH1-L10-Fc; VL1-L11-VL2-L12-VH2-L13-VH1-L14-CL-L15-Fc; VL1-L11-VH2-L12-VL2-L13-VH1-L14-CL-L15-Fc; VH1-L16-VH2-L17-VL2-L18-VL1-L19-CL-L20-Fc; VH1-L16-VL2-L17-VH2-L18-VL1-L19-CL-L20-Fc; VL1-L21-VL2-L22-VH2-L23-VH1-L24-CH1-L25-CL-L26-Fc; VL1-L21-VH2-L22-VL2-L23-VH1-L24-CH1-L25-CL-L26-Fc; VH1-L27-VH2-L28-VL2-L29-VL1-L30-CH1-L31-CL-L32-Fc; VH1-L27-VL2-L28-VH2-L29-VL1-L30-CH1-L31-CL-L32-Fc; VL1-L33-VL2-L34-VH2-L35-VH1-L36-CL-L37-CH1-L38-Fc; VL1-L33-VH2-L34-VL2-L35-VH1-L36-CL-L37-CH1-L38-Fc; VH1-L39-VH2-L40-VL2-L41-VL1-L42-CL-L43-CH1-L44-Fc; or VH1-L39-VL2-L40-VH2-L41-VL1-L42-CL-L43-CH1-L44-Fc; and the second polypeptide has a structure represented by VH3-L50-VL4-L51-VH4-L52-VL3-L53-CH1-L54-Fc. In some aspects, the first polypeptide has a structure represented by VL1-L1-VL2-L2-VH2-L3-VH1-L4-CH1-L5-Fc; VL1-L1-VH2-L2-VL2-L3-VH1-L4-CH1-L5-Fc; VH1-L6-VH2-L7-VL2-L8-VL1-L9-CH1-L10-Fc; VH1-L6-VL2-L7-VH2-L8-VL1-L9-CH1-L10-Fc; VL1-L11-VL2-L12-VH2-L13-VH1-L14-CL-L15-Fc; VL1-L11-VH2-L12-VL2-L13-VH1-L14-CL-L15-Fc; VH1-L16-VH2-L17-VL2-L18-VL1-L19-CL-L20-Fc; VH1-L16-VL2-L17-VH2-L18-VL1-L19-CL-L20-Fc; VL1-L21-VL2-L22-VH2-L23-VH1-L24-CH1-L25-CL-L26-Fc; VL1-L21-VH2-L22-VL2-L23-VH1-L24-CH1-L25-CL-L26-Fc; VH1-L27-VH2-L28-VL2-L29-VL1-L30-CH1-L31-CL-L32-Fc; VH1-L27-VL2-L28-VH2-L29-VL1-L30-CH1-L31-CL-L32-Fc; VL1-L33-VL2-L34-VH2-L35-VH1-L36-CL-L37-CH1-L38-Fc; VL1-L33-VH2-L34-VL2-L35-VH1-L36-CL-L37-CH1-L38-Fc; VH1-L39-VH2-L40-VL2-L41-VL1-L42-CL-L43-CH1-L44-Fc; or VH1-L39-VL2-L40-VH2-L41-VL1-L42-CL-L43-CH1-L44-Fc; and the second polypeptide has a structure represented by VL3-L55-VL4-L56-VH4-L57-VH3-L58-CL-L59-Fc. In some aspects, the first polypeptide has a structure represented by VL1-L1-VL2-L2-VH2-L3-VH1-L4-CH1-L5-Fc; VL1-L1-VH2-L2-VL2-L3-VH1-L4-CH1-L5-Fc; VH1-L6-VH2-L7-VL2-L8-VL1-L9-CH1-L10-Fc; VH1-L6-VL2-L7-VH2-L8-VL1-L9-CH1-L10-Fc; VL1-L11-VL2-L12-VH2-L13-VH1-L14-CL-L15-Fc; VL1-L11-VH2-L12-VL2-L13-VH1-L14-CL-L15-Fc; VH1-L16-VH2-L17-VL2-L18-VL1-L19-CL-L20-Fc; VH1-L16-VL2-L17-VH2-L18-VL1-L19-CL-L20-Fc; VL1-L21-VL2-L22-VH2-L23-VH1-L24-CH1-L25-CL-L26-Fc; VL1-L21-VH2-L22-VL2-L23-VH1-L24-CH1-L25-CL-L26-Fc; VH1-L27-VH2-L28-VL2-L29-VL1-L30-CH1-L31-CL-L32-Fc; VH1-L27-VL2-L28-VH2-L29-VL1-L30-CH1-L31-CL-L32-Fc; VL1-L33-VL2-L34-VH2-L35-VH1-L36-CL-L37-CH1-L38-Fc; VL1-L33-VH2-L34-VL2-L35-VH1-L36-CL-L37-CH1-L38-Fc; VH1-L39-VH2-L40-VL2-L41-VL1-L42-CL-L43-CH1-L44-Fc; or VH1-L39-VL2-L40-VH2-L41-VL1-L42-CL-L43-CH1-L44-Fc; and the second polypeptide has a structure represented by VL3-L55-VH4-L56-VL4-L57-VH3-L58-CL-L59-Fc. In some aspects, the first polypeptide has a structure represented by VL1-L1-VL2-L2-VH2-L3-VH1-L4-CH1-L5-Fc; VL1-L1-VH2-L2-VL2-L3-VH1-L4-CH1-L5-Fc; VH1-L6-VH2-L7-VL2-L8-VL1-L9-CH1-L10-Fc; VH1-L6-VL2-L7-VH2-L8-VL1-L9-CH1-L10-Fc; VL1-L11-VL2-L12-VH2-L13-VH1-L14-CL-L15-Fc; VL1-L11-VH2-L12-VL2-L13-VH1-L14-CL-L15-Fc; VH1-L16-VH2-L17-VL2-L18-VL1-L19-CL-L20-Fc; VH1-L16-VL2-L17-VH2-L18-VL1-L19-CL-L20-Fc; VL1-L21-VL2-L22-VH2-L23-VH1-L24-CH1-L25-CL-L26-Fc; VL1-L21-VH2-L22-VL2-L23-VH1-L24-CH1-L25-CL-L26-Fc; VH1-L27-VH2-L28-VL2-L29-VL1-L30-CH1-L31-CL-L32-Fc; VH1-L27-VL2-L28-VH2-L29-VL1-L30-CH1-L31-CL-L32-Fc; VL1-L33-VL2-L34-VH2-L35-VH1-L36-CL-L37-CH1-L38-Fc; VL1-L33-VH2-L34-VL2-L35-VH1-L36-CL-L37-CH1-L38-Fc; VH1-L39-VH2-L40-VL2-L41-VL1-L42-CL-L43-CH1-L44-Fc; or VH1-L39-VL2-L40-VH2-L41-VL1-L42-CL-L43-CH1-L44-Fc; and the second polypeptide has a structure represented by VH3-L60-VH4-L61-VL4-L62-VL3-L63-CL-L64-Fc. In some aspects, the first polypeptide has a structure represented by VL1-L1-VL2-L2-VH2-L3-VH1-L4-CH1-L5-Fc; VL1-L1-VH2-L2-VL2-L3-VH1-L4-CH1-L5-Fc; VH1-L6-VH2-L7-VL2-L8-VL1-L9-CH1-L10-Fc; VH1-L6-VL2-L7-VH2-L8-VL1-L9-CH1-L10-Fc; VL1-L11-VL2-L12-VH2-L13-VH1-L14-CL-L15-Fc; VL1-L11-VH2-L12-VL2-L13-VH1-L14-CL-L15-Fc; VH1-L16-VH2-L17-VL2-L18-VL1-L19-CL-L20-Fc; VH1-L16-VL2-L17-VH2-L18-VL1-L19-CL-L20-Fc; VL1-L21-VL2-L22-VH2-L23-VH1-L24-CH1-L25-CL-L26-Fc; VL1-L21-VH2-L22-VL2-L23-VH1-L24-CH1-L25-CL-L26-Fc; VH1-L27-VH2-L28-VL2-L29-VL1-L30-CH1-L31-CL-L32-Fc; VH1-L27-VL2-L28-VH2-L29-VL1-L30-CH1-L31-CL-L32-Fc; VL1-L33-VL2-L34-VH2-L35-VH1-L36-CL-L37-CH1-L38-Fc; VL1-L33-VH2-L34-VL2-L35-VH1-L36-CL-L37-CH1-L38-Fc; VH1-L39-VH2-L40-VL2-L41-VL1-L42-CL-L43-CH1-L44-Fc; or VH1-L39-VL2-L40-VH2-L41-VL1-L42-CL-L43-CH1-L44-Fc; and the second polypeptide has a structure represented by VH3-L60-VL4-L61-VH4-L62-VL3-L63-CL-L64-Fc. In some aspects, the first polypeptide has a structure represented by VL1-L1-VL2-L2-VH2-L3-VH1-L4-CH1-L5-Fc; VL1-L1-VH2-L2-VL2-L3-VH1-L4-CH1-L5-Fc; VH1-L6-VH2-L7-VL2-L8-VL1-L9-CH1-L10-Fc; VH1-L6-VL2-L7-VH2-L8-VL1-L9-CH1-L10-Fc; VL1-L11-VL2-L12-VH2-L13-VH1-L14-CL-L15-Fc; VL1-L11-VH2-L12-VL2-L13-VH1-L14-CL-L15-Fc; VH1-L16-VH2-L17-VL2-L18-VL1-L19-CL-L20-Fc; VH1-L16-VL2-L17-VH2-L18-VL1-L19-CL-L20-Fc; VL1-L21-VL2-L22-VH2-L23-VH1-L24-CH1-L25-CL-L26-Fc; VL1-L21-VH2-L22-VL2-L23-VH1-L24-CH1-L25-CL-L26-Fc; VH1-L27-VH2-L28-VL2-L29-VL1-L30-CH1-L31-CL-L32-Fc; VH1-L27-VL2-L28-VH2-L29-VL1-L30-CH1-L31-CL-L32-Fc; VL1-L33-VL2-L34-VH2-L35-VH1-L36-CL-L37-CH1-L38-Fc; VL1-L33-VH2-L34-VL2-L35-VH1-L36-CL-L37-CH1-L38-Fc; VH1-L39-VH2-L40-VL2-L41-VL1-L42-CL-L43-CH1-L44-Fc; or VH1-L39-VL2-L40-VH2-L41-VL1-L42-CL-L43-CH1-L44-Fc; and the second polypeptide has a structure represented by VL3-L65-VL4-L66-VH4-L67-VH3-L68-CH1-L69-CL-L70-Fc. In some aspects, the first polypeptide has a structure represented by VL1-L1-VL2-L2-VH2-L3-VH1-L4-CH1-L5-Fc; VL1-L1-VH2-L2-VL2-L3-VH1-L4-CH1-L5-Fc; VH1-L6-VH2-L7-VL2-L8-VL1-L9-CH1-L10-Fc; VH1-L6-VL2-L7-VH2-L8-VL1-L9-CH1-L10-Fc; VL1-L11-VL2-L12-VH2-L13-VH1-L14-CL-L15-Fc; VL1-L11-VH2-L12-VL2-L13-VH1-L14-CL-L15-Fc; VH1-L16-VH2-L17-VL2-L18-VL1-L19-CL-L20-Fc; VH1-L16-VL2-L17-VH2-L18-VL1-L19-CL-L20-Fc; VL1-L21-VL2-L22-VH2-L23-VH1-L24-CH1-L25-CL-L26-Fc; VL1-L21-VH2-L22-VL2-L23-VH1-L24-CH1-L25-CL-L26-Fc; VH1-L27-VH2-L28-VL2-L29-VL1-L30-CH1-L31-CL-L32-Fc; VH1-L27-VL2-L28-VH2-L29-VL1-L30-CH1-L31-CL-L32-Fc; VL1-L33-VL2-L34-VH2-L35-VH1-L36-CL-L37-CH1-L38-Fc; VL1-L33-VH2-L34-VL2-L35-VH1-L36-CL-L37-CH1-L38-Fc; VH1-L39-VH2-L40-VL2-L41-VL1-L42-CL-L43-CH1-L44-Fc; or VH1-L39-VL2-L40-VH2-L41-VL1-L42-CL-L43-CH1-L44-Fc; and the second polypeptide has a structure represented by VL3-L65-VH4-L66-VL4-L67-VH3-L68-CH1-L69-CL-L70-Fc. In some aspects, the first polypeptide has a structure represented by VL1-L1-VL2-L2-VH2-L3-VH1-L4-CH1-L5-Fc; VL1-L1-VH2-L2-VL2-L3-VH1-L4-CH1-L5-Fc; VH1-L6-VH2-L7-VL2-L8-VL1-L9-CH1-L10-Fc; VH1-L6-VL2-L7-VH2-L8-VL1-L9-CH1-L10-Fc; VL1-L11-VL2-L12-VH2-L13-VH1-L14-CL-L15-Fc; VL1-L11-VH2-L12-VL2-L13-VH1-L14-CL-L15-Fc; VH1-L16-VH2-L17-VL2-L18-VL1-L19-CL-L20-Fc; VH1-L16-VL2-L17-VH2-L18-VL1-L19-CL-L20-Fc; VL1-L21-VL2-L22-VH2-L23-VH1-L24-CH1-L25-CL-L26-Fc; VL1-L21-VH2-L22-VL2-L23-VH1-L24-CH1-L25-CL-L26-Fc; VH1-L27-VH2-L28-VL2-L29-VL1-L30-CH1-L31-CL-L32-Fc; VH1-L27-VL2-L28-VH2-L29-VL1-L30-CH1-L31-CL-L32-Fc; VL1-L33-VL2-L34-VH2-L35-VH1-L36-CL-L37-CH1-L38-Fc; VL1-L33-VH2-L34-VL2-L35-VH1-L36-CL-L37-CH1-L38-Fc; VH1-L39-VH2-L40-VL2-L41-VL1-L42-CL-L43-CH1-L44-Fc; or VH1-L39-VL2-L40-VH2-L41-L1-L42-CL-L43-CH1-L44-Fc; and the second polypeptide has a structure represented by VH3-L71-VH4-L72-VL4-L73-VL3-L74-CH1-L75-CL-L76-Fc. In some aspects, the first polypeptide has a structure represented by VL1-L1-VL2-L2-VH2-L3-VH1-L4-CH1-L5-Fc; VL1-L1-VH2-L2-VL2-L3-VH1-L4-CH1-L5-Fc; VH1-L6-VH2-L7-VL2-L8-VL1-L9-CH1-L10-Fc; VH1-L6-VL2-L7-VH2-L8-VL1-L9-CH1-L10-Fc; VL1-L11-VL2-L12-VH2-L13-VH1-L14-CL-L15-Fc; VL1-L11-VH2-L12-VL2-L13-VH1-L14-CL-L15-Fc; VH1-L16-VH2-L17-VL2-L18-VL1-L19-CL-L20-Fc; VH1-L16-VL2-L17-VH2-L18-VL1-L19-CL-L20-Fc; VL1-L21-VL2-L22-VH2-L23-VH1-L24-CH1-L25-CL-L26-Fc; VL1-L21-VH2-L22-VL2-L23-VH1-L24-CH1-L25-CL-L26-Fc; VH1-L27-VH2-L28-VL2-L29-VL1-L30-CH1-L31-CL-L32-Fc; VH1-L27-VL2-L28-VH2-L29-VL1-L30-CH1-L31-CL-L32-Fc; VL1-L33-VL2-L34-VH2-L35-VH1-L36-CL-L37-CH1-L38-Fc; VL1-L33-VH2-L34-VL2-L35-VH1-L36-CL-L37-CH1-L38-Fc; VH1-L39-VH2-L40-VL2-L41-VL1-L42-CL-L43-CH1-L44-Fc; or VH1-L39-VL2-L40-VH2-L41-VL1-L42-CL-L43-CH1-L44-Fc; and the second polypeptide has a structure represented by VH3-L71-VL4-L72-VH4-L73-VL3-L74-CH1-L75-CL-L76-Fc. In some aspects, the first polypeptide has a structure represented by VL1-L1-VL2-L2-VH2-L3-VH1-L4-CH1-L5-Fc; VL1-L1-VH2-L2-VL2-L3-VH1-L4-CH1-L5-Fc; VH1-L6-VH2-L7-VL2-L8-VL1-L9-CH1-L10-Fc; VH1-L6-VL2-L7-VH2-L8-VL1-L9-CH1-L10-Fc; VL1-L11-VL2-L12-VH2-L13-VH1-L14-CL-L15-Fc; VL1-L11-VH2-L12-VL2-L13-VH1-L14-CL-L15-Fc; VH1-L16-VH2-L17-VL2-L18-VL1-L19-CL-L20-Fc; VH1-L16-VL2-L17-VH2-L18-VL1-L19-CL-L20-Fc; VL1-L21-VL2-L22-VH2-L23-VH1-L24-CH1-L25-CL-L26-Fc; VL1-L21-VH2-L22-VL2-L23-VH1-L24-CH1-L25-CL-L26-Fc; VH1-L27-VH2-L28-VL2-L29-VL1-L30-CH1-L31-CL-L32-Fc; VH1-L27-VL2-L28-VH2-L29-VL1-L30-CH1-L31-CL-L32-Fc; VL1-L33-VL2-L34-VH2-L35-VH1-L36-CL-L37-CH1-L38-Fc; VL1-L33-VH2-L34-VL2-L35-VH1-L36-CL-L37-CH1-L38-Fc; VH1-L39-VH2-L40-VL2-L41-VL1-L42-CL-L43-CH1-L44-Fc; or VH1-L39-VL2-L40-VH2-L41-VL1-L42-CL-L43-CH1-L44-Fc; and the second polypeptide has a structure represented by VL3-L77-VL4-L78-VH4-L79-VH3-L80-CL-L81-CH1-L82-Fc. In some aspects, the first polypeptide has a structure represented by VL1-L1-VL2-L2-VH2-L3-VH1-L4-CH1-L5-Fc; VL1-L1-VH2-L2-VL2-L3-VH1-L4-CH1-L5-Fc; VH1-L6-VH2-L7-VL2-L8-VL1-L9-CH1-L10-Fc; VH1-L6-VL2-L7-VH2-L8-VL1-L9-CH1-L10-Fc; VL1-L11-VL2-L12-VH2-L13-VH1-L14-CL-L15-Fc; VL1-L11-VH2-L12-VL2-L13-VH1-L14-CL-L15-Fc; VH1-L16-VH2-L17-VL2-L18-VL1-L19-CL-L20-Fc; VH1-L16-VL2-L17-VH2-L18-VL1-L19-CL-L20-Fc; VL1-L21-VL2-L22-VH2-L23-VH1-L24-CH1-L25-CL-L26-Fc; VL1-L21-VH2-L22-VL2-L23-VH1-L24-CH1-L25-CL-L26-Fc; VH1-L27-VH2-L28-VL2-L29-VL1-L30-CH1-L31-CL-L32-Fc; VH1-L27-VL2-L28-VH2-L29-VL1-L30-CH1-L31-CL-L32-Fc; VL1-L33-VL2-L34-VH2-L35-VH1-L36-CL-L37-CH1-L38-Fc; VL1-L33-VH2-L34-VL2-L35-VH1-L36-CL-L37-CH1-L38-Fc; VH1-L39-VH2-L40-VL2-L41-VL1-L42-CL-L43-CH1-L44-Fc; or VH1-L39-VL2-L40-VH2-L41-VL1-L42-CL-L43-CH1-L44-Fc; and the second polypeptide has a structure represented by VL3-L77-VH4-L78-VL4-L79-VH3-L80-CL-L81-CH1-L82-Fc. In some aspects, the first polypeptide has a structure represented by VL1-L1-VL2-L2-VH2-L3-VH1-L4-CH1-L5-Fc; VL1-L1-VH2-L2-VL2-L3-VH1-L4-CH1-L5-Fc; VH1-L6-VH2-L7-VL2-L8-VL1-L9-CH1-L10-Fc; VH1-L6-VL2-L7-VH2-L8-VL1-L9-CH1-L10-Fc; VL1-L11-VL2-L12-VH2-L13-VH1-L14-CL-L15-Fc; VL1-L11-VH2-L12-VL2-L13-VH1-L14-CL-L15-Fc; VH1-L16-VH2-L17-VL2-L18-VL1-L19-CL-L20-Fc; VH1-L16-VL2-L17-VH2-L18-VL1-L19-CL-L20-Fc; VL1-L21-VL2-L22-VH2-L23-VH1-L24-CH1-L25-CL-L26-Fc; VL1-L21-VH2-L22-VL2-L23-VH1-L24-CH1-L25-CL-L26-Fc; VH1-L27-VH2-L28-VL2-L29-VL1-L30-CH1-L31-CL-L32-Fc; VH1-L27-VL2-L28-VH2-L29-VL1-L30-CH1-L31-CL-L32-Fc; VL1-L33-VL2-L34-VH2-L35-VH1-L36-CL-L37-CH1-L38-Fc; VL1-L33-VH2-L34-VL2-L35-VH1-L36-CL-L37-CH1-L38-Fc; VH1-L39-VH2-L40-VL2-L41-VL1-L42-CL-L43-CH1-L44-Fc; or VH1-L39-VL2-L40-VH2-L41-VL1-L42-CL-L43-CH1-L44-Fc; and the second polypeptide has a structure represented by VH3-L83-VH4-L84-VL4-L85-VL3-L86-CL-L87-CH1-L88-Fc. In some aspects, the first polypeptide has a structure represented by VL1-L1-VL2-L2-VH2-L3-VH1-L4-CH1-L5-Fc; VL1-L1-VH2-L2-VL2-L3-VH1-L4-CH1-L5-Fc; VH1-L6-VH2-L7-VL2-L8-VL1-L9-CH1-L10-Fc; VH1-L6-VL2-L7-VH2-L8-VL1-L9-CH1-L10-Fc; VL1-L11-VL2-L12-VH2-L13-VH1-L14-CL-L15-Fc; VL1-L11-VH2-L12-VL2-L13-VH1-L14-CL-L15-Fc; VH1-L16-VH2-L17-VL2-L18-VL1-L19-CL-L20-Fc; VH1-L16-VL2-L17-VH2-L18-VL1-L19-CL-L20-Fc; VL1-L21-VL2-L22-VH2-L23-VH1-L24-CH1-L25-CL-L26-Fc; VL1-L21-VH2-L22-VL2-L23-VH1-L24-CH1-L25-CL-L26-Fc; VH1-L27-VH2-L28-VL2-L29-VL1-L30-CH1-L31-CL-L32-Fc; VH1-L27-VL2-L28-VH2-L29-VL1-L30-CH1-L31-CL-L32-Fe; VL1-L33-VL2-L34-VH2-L35-VH1-L36-CL-L37-CH1-L38-Fc; VL1-L33-VH2-L34-VL2-L35-VH1-L36-CL-L37-CH1-L38-Fc; VH1-L39-VH2-L40-VL2-L41-VL1-L42-CL-L43-CH1-L44-Fc; or VH1-L39-VL2-L40-VH2-L41-VL1-L42-CL-L43-CH1-L44-Fc; and the second polypeptide has a structure represented by VH3-L83-VL4-L84-VH4-L85-VL3-L86-CL-L87-CH1-L88-Fc. In some aspects, the first polypeptide has a structure represented by: VL1-L1-VL2-L2-VH2-L3-VH1-L4-CH1-L5-Fc; VH1-L6-VH2-L7-VL2-L8-VL1-L9-CH1-L10-Fc; VL1-L11-VL2-L12-VH2-L13-VH1-L14-CL-L15-Fc; VH1-L16-VH2-L17-VL2-L18-VL1-L19-CL-L20-Fc; VL1-L21-VL2-L22-VH2-L23-VH1-L24-CH1-L25-CL-L26-Fc; VH1-L27-VH2-L28-VL2-L29-VL1-L30-CH1-L31-CL-L32-Fc; VL1-L33-VL2-L34-VH2-L35-VH1-L36-CL-L37-CH1-L38-Fc; or VH1-L39-VH2-L40-VL2-L41-VL1-L42-CL-L43-CH1-L44-Fc; and the second polypeptide has a structure represented by: VL3-L45-VL4-L46-VH4-L47-VH3-L48-CH1-L49-Fc; VH3-L50-VH4-L51-VL4-L52-VL3-L53-CH1-L54-Fc; VL3-L55-VL4-L56-VH4-L57-VH3-L58-CL-L59-Fc; VH3-L60-VH4-L61-VL4-L62-VL3-L63-CL-L64-Fc; VL3-L65-VL4-L66-VH4-L67-VH3-L68-CH1-L69-CL-L70-Fc; VH3-L71-VH4-L72-VL4-L73-VL3-L74-CH1-L75-CL-L76-Fc; VL3-L77-VL4-L78-VH4-L79-VH3-L80-CL-L81-CH1-L82-Fc; or VH3-L83-VH4-L84-VL4-L85-VL3-L86-CL-L87-CH1-L88-Fc.

Any one of the first polypeptides described herein may be combined with any one of the second, third and/or fourth polypeptides described herein to form an antigen binding polypeptide complex of the invention.

All the disclosures relating to the antigen binding polypeptide complex structures described herein apply to and can be combined with all the VH and VL regions described herein including all the target antigens described herein and all the VH and VL sequences and CDR sequences described herein.

In some aspects, the antigen binding polypeptide complex (e.g., antibody or antigen binding fragment thereof) specifically binds to a tumor-associated antigen (TAA). In some aspects, the antigen binding polypeptide complex specifically binds at least one epitope on a TAA. In some aspects, a light chain variable region (VL) and a corresponding heavy chain variable region (VH) of the antigen binding polypeptide complex specifically bind to a TAA. In some aspects, the TAA is A2AR, APRIL, ATPDase, BAFF, BAFFR, BCMA, BlyS, BTK, BTLA, B7DC, B7H1, B7H2, B7H3, B7H4, B7H5, B7H6, B7H7, B7RP1, B7-4, C3, C5, CCL2, CCL3, CCL4, CCL5, CCL7, CCL8, CCL11, CCL15, CCL17, CCL19, CCL20, CCL21, CCL25, CCR3, CCR4, CD16A, CD19, CD20, CD24, CD27, CD28, CD30, CD38, CD39, CD40, CD40L, CD47, CD52, CD70, CD80, CD86, CD123, CD133, CD137, CD137L, CD160, CD272, CEACAM5, CLEC9, CLEC91, CRTH2, CSF-1, CSF-2, CSF-3, CXCL1, CXCL2, CXCL4, CXCL12, CXCL13, CXCR3, cMet, CTLA4, DLL3, DLL4, DNGR-1, E-cadherin, EGFR, ENTPD1, EpCAM, FCER1, FCER1A, FCER2, FGFR, FLAP, FOLH1, Gi24, GITR, GITRL, GPR5, GP100, GPRC5D, HER2, HER3, ICOSL, ICOS, HHLA2, HMGB1, HVEM, IDO, IFNa, IgE, IGF1R, IL2Rbeta, IL1, ILIA, IL1B, IL1F10, IL2, IL4, IL4Ra, IL5, IL5R, IL6, IL7, IL7Ra, IL8, IL9, IL9R, IL10, rhILlO, IL12, IL13, IL13Ra1, IL13Ra2, IL15, IL17, IL17Rb, IL18, IL22, IL23, IL25, IL7, IL33, IL35, ITGB4, ITK, KIR, LAG3, LAMP1, leptin, LPFS2, MHC class II, MUC-1, MUC-16, NCR3LG1, NKG2D, NKp46, NTPDase-1, OX40, OX40L, PD-1, PD-L1, PD-L2, PROM1, S152, SIRPalpha, SISP1, SLC, SPG64, ST2, STEAP1, STEAP2, Syk kinase, STEAP1, TROP2, TACI, TDO, TGFBETA, T14, TIGIT, TIM3, TLR, TLR2, TLR4, TLR5, TLR9, TMEF1, TNFa, TNFRSF7, Tp55, TREM1, TSLP, TSLPR, TWEAK, VEGF, VISTA, Vstm3, or WUCAM, or combinations thereof. In some aspects, the antigen binding polypeptide complex comprises a VL1 that specifically binds to A2AR, APRIL, ATPDase, BAFF, BAFFR, BCMA, BlyS, BTK, BTLA, B7DC, B7H1, B7H2, B7H3, B7H4, B7H5, B7H6, B7H7, B7RP1, B7-4, C3, C5, CCL2, CCL3, CCL4, CCL5, CCL7, CCL8, CCL11, CCL15, CCL17, CCL19, CCL20, CCL21, CCL25, CCR3, CCR4, CD16A, CD19, CD20, CD24, CD27, CD28, CD30, CD38, CD39, CD40, CD40L, CD47, CD52, CD70, CD80, CD86, CD123, CD133, CD137, CD137L, CD160, CD272, CEACAM5, CLEC9, CLEC91, CRTH2, CSF-1, CSF-2, CSF-3, CXCL1, CXCL2, CXCL4, CXCL12, CXCL13, CXCR3, cMet, CTLA4, DLL3, DLL4, DNGR-1, E-cadherin, EGFR, ENTPD1, EpCAM, FCER1, FCER1A, FCER2, FGFR, FLAP, FOLH1, Gi24, GITR, GITRL, GPR5, GP100, GPRC5D, HER2, HER3, ICOSL, ICOS, HHLA2, HMGB1, HVEM, IDO, IFNa, IgE, IGF1R, IL2Rbeta, IL1, ILIA, IL1B, IL1F10, IL2, IL4, IL4Ra, IL5, IL5R, IL6, IL7, IL7Ra, IL8, IL9, IL9R, IL10, rhILlO, IL12, IL13, IL13Ra1, IL13Ra2, IL15, IL17, IL17Rb, IL18, IL22, IL23, IL25, IL7, IL33, IL35, ITGB4, ITK, KIR, LAG3, LAMP1, leptin, LPFS2, MHC class II, MUC-1, MUC-16, NCR3LG1, NKG2D, NKp46, NTPDase-1, OX40, OX40L, PD-1, PD-L1, PD-L2, PROM1, S152, SIRPalpha, SISP1, SLC, SPG64, ST2, STEAP1, STEAP2, Syk kinase, STEAP1, TROP2, TACI, TDO, TGFBETA, T14, TIGIT, TIM3, TLR, TLR2, TLR4, TLR5, TLR9, TMEF1, TNFa, TNFRSF7, Tp55, TREM1, TSLP, TSLPR, TWEAK, VEGF, VISTA, Vstm3, or WUCAM, or combinations thereof. For example, the VL1 may specifically bind to CD3. For example, the VL1 may specifically bind to Trop2. For example, the VL1 may specifically bind to cMet. For example, the VL1 may specifically bind to CD19. For example, the VL1 may specifically bind to CD20. In some aspects, the antigen binding polypeptide complex comprises a VL2 that specifically binds to A2AR, APRIL, ATPDase, BAFF, BAFFR, BCMA, BlyS, BTK, BTLA, B7DC, B7H1, B7H2, B7H3, B7H4, B7H5, B7H6, B7H7, B7RP1, B7-4, C3, C5, CCL2, CCL3, CCL4, CCL5, CCL7, CCL8, CCL11, CCL15, CCL17, CCL19, CCL20, CCL21, CCL25, CCR3, CCR4, CD16A, CD19, CD20, CD24, CD27, CD28, CD30, CD38, CD39, CD40, CD40L, CD47, CD52, CD70, CD80, CD86, CD123, CD133, CD137, CD137L, CD160, CD272, CEACAM5, CLEC9, CLEC91, CRTH2, CSF-1, CSF-2, CSF-3, CXCL1, CXCL2, CXCL4, CXCL12, CXCL13, CXCR3, cMet, CTLA4, DLL3, DLL4, DNGR-1, E-cadherin, EGFR, ENTPD1, EpCAM, FCER1, FCER1A, FCER2, FGFR, FLAP, FOLH1, Gi24, GITR, GITRL, GPR5, GP100, GPRC5D, HER2, HER3, ICOSL, ICOS, HHLA2, HMGB1, HVEM, IDO, IFNa, IgE, IGF1R, IL2Rbeta, IL1, ILIA, IL1B, IL1F10, IL2, IL4, IL4Ra, IL5, IL5R, IL6, IL7, IL7Ra, IL8, IL9, IL9R, IL10, rhILlO, IL12, IL13, IL13Ra1, IL13Ra2, IL15, IL17, IL17Rb, IL18, IL22, IL23, IL25, IL7, IL33, IL35, ITGB4, ITK, KIR, LAG3, LAMP1, leptin, LPFS2, MHC class II, MUC-1, MUC-16, NCR3LG1, NKG2D, NKp46, NTPDase-1, OX40, OX40L, PD-1, PD-L1, PD-L2, PROM1, S152, SIRPalpha, SISP1, SLC, SPG64, ST2, STEAP1, STEAP2, Syk kinase, STEAP1, TROP2, TACI, TDO, TGFBETA, T14, TIGIT, TIM3, TLR, TLR2, TLR4, TLR5, TLR9, TMEF1, TNFa, TNFRSF7, Tp55, TREM1, TSLP, TSLPR, TWEAK, VEGF, VISTA, Vstm3, or WUCAM, or combinations thereof. For example, the VL2 may specifically bind to CD3. For example, the VL2 may specifically bind to Trop2. For example, the VL2 may specifically bind to cMet. For example, the VL2 may specifically bind to CD19. For example, the VL2 may specifically bind to CD20. In some aspects, the antigen binding polypeptide complex comprises a VL3 that specifically binds to A2AR, APRIL, ATPDase, BAFF, BAFFR, BCMA, BlyS, BTK, BTLA, B7DC, B7H1, B7H2, B7H3, B7H4, B7H5, B7H6, B7H7, B7RP1, B7-4, C3, C5, CCL2, CCL3, CCL4, CCL5, CCL7, CCL8, CCL11, CCL15, CCL17, CCL19, CCL20, CCL21, CCL25, CCR3, CCR4, CD16A, CD19, CD20, CD24, CD27, CD28, CD30, CD38, CD39, CD40, CD40L, CD47, CD52, CD70, CD80, CD86, CD123, CD133, CD137, CD137L, CD160, CD272, CEACAM5, CLEC9, CLEC91, CRTH2, CSF-1, CSF-2, CSF-3, CXCL1, CXCL2, CXCL4, CXCL12, CXCL13, CXCR3, cMet, CTLA4, DLL3, DLL4, DNGR-1, E-cadherin, EGFR, ENTPD1, EpCAM, FCER1, FCER1A, FCER2, FGFR, FLAP, FOLH1, Gi24, GITR, GITRL, GPR5, GP100, GPRC5D, HER2, HER3, ICOSL, ICOS, HHLA2, HMGB1, HVEM, IDO, IFNa, IgE, IGF1R, IL2Rbeta, IL1, ILIA, IL1B, IL1F10, IL2, IL4, IL4Ra, IL5, IL5R, IL6, IL7, IL7Ra, IL8, IL9, IL9R, IL10, rhILlO, IL12, IL13, IL13Ra1, IL13Ra2, IL15, IL17, IL17Rb, IL18, IL22, IL23, IL25, IL7, IL33, IL35, ITGB4, ITK, KIR, LAG3, LAMP1, leptin, LPFS2, MHC class II, MUC-1, MUC-16, NCR3LG1, NKG2D, NKp46, NTPDase-1, OX40, OX40L, PD-1, PD-L1, PD-L2, PROM1, S152, SIRPalpha, SISP1, SLC, SPG64, ST2, STEAP1, STEAP2, Syk kinase, STEAP1, TROP2, TACI, TDO, TGFBETA, T14, TIGIT, TIM3, TLR, TLR2, TLR4, TLR5, TLR9, TMEF1, TNFa, TNFRSF7, Tp55, TREM1, TSLP, TSLPR, TWEAK, VEGF, VISTA, Vstm3, or WUCAM, or combinations thereof. For example, the VL3 may specifically bind to CD3. For example, the VL3 may specifically bind to Trop2. For example, the VL3 may specifically bind to cMet. For example, the VL3 may specifically bind to CD19. For example, the VL3 may specifically bind to CD20. In some aspects, the antigen binding polypeptide complex comprises a VL4 that specifically binds to A2AR, APRIL, ATPDase, BAFF, BAFFR, BCMA, BlyS, BTK, BTLA, B7DC, B7H1, B7H2, B7H3, B7H4, B7H5, B7H6, B7H7, B7RP1, B7-4, C3, C5, CCL2, CCL3, CCL4, CCL5, CCL7, CCL8, CCL11, CCL15, CCL17, CCL19, CCL20, CCL21, CCL25, CCR3, CCR4, CD16A, CD19, CD20, CD24, CD27, CD28, CD30, CD38, CD39, CD40, CD40L, CD47, CD52, CD70, CD80, CD86, CD123, CD133, CD137, CD137L, CD160, CD272, CEACAM5, CLEC9, CLEC91, CRTH2, CSF-1, CSF-2, CSF-3, CXCL1, CXCL2, CXCL4, CXCL12, CXCL13, CXCR3, cMet, CTLA4, DLL3, DLL4, DNGR-1, E-cadherin, EGFR, ENTPD1, EpCAM, FCER1, FCER1A, FCER2, FGFR, FLAP, FOLH1, Gi24, GITR, GITRL, GPR5, GP100, GPRC5D, HER2, HER3, ICOSL, ICOS, HHLA2, HMGB1, HVEM, IDO, IFNa, IgE, IGF1R, IL2Rbeta, IL1, ILIA, IL1B, IL1F10, IL2, IL4, IL4Ra, IL5, IL5R, IL6, IL7, IL7Ra, IL8, IL9, IL9R, IL10, rhILlO, IL12, IL13, IL13Ra1, IL13Ra2, IL15, IL17, IL17Rb, IL18, IL22, IL23, IL25, IL7, IL33, IL35, ITGB4, ITK, KIR, LAG3, LAMP1, leptin, LPFS2, MHC class II, MUC-1, MUC-16, NCR3LG1, NKG2D, NKp46, NTPDase-1, OX40, OX40L, PD-1, PD-L1, PD-L2, PROM1, S152, SIRPalpha, SISP1, SLC, SPG64, ST2, STEAP1, STEAP2, Syk kinase, STEAP1, TROP2, TACI, TDO, TGFBETA, T14, TIGIT, TIM3, TLR, TLR2, TLR4, TLR5, TLR9, TMEF1, TNFa, TNFRSF7, Tp55, TREM1, TSLP, TSLPR, TWEAK, VEGF, VISTA, Vstm3, or WUCAM, or combinations thereof. For example, the VL4 may specifically bind to CD3. For example, the VL4 may specifically bind to Trop2. For example, the VL4 may specifically bind to cMet. For example, the VL4 may specifically bind to CD19. For example, the VL4 may specifically bind to CD20. In some aspects, the antigen binding polypeptide complex comprises a VH1 that specifically binds to A2AR, APRIL, ATPDase, BAFF, BAFFR, BCMA, BlyS, BTK, BTLA, B7DC, B7H1, B7H2, B7H3, B7H4, B7H5, B7H6, B7H7, B7RP1, B7-4, C3, C5, CCL2, CCL3, CCL4, CCL5, CCL7, CCL8, CCL11, CCL15, CCL17, CCL19, CCL20, CCL21, CCL25, CCR3, CCR4, CD16A, CD19, CD20, CD24, CD27, CD28, CD30, CD38, CD39, CD40, CD40L, CD47, CD52, CD70, CD80, CD86, CD123, CD133, CD137, CD137L, CD160, CD272, CEACAM5, CLEC9, CLEC91, CRTH2, CSF-1, CSF-2, CSF-3, CXCL1, CXCL2, CXCL4, CXCL12, CXCL13, CXCR3, cMet, CTLA4, DLL3, DLL4, DNGR-1, E-cadherin, EGFR, ENTPD1, EpCAM, FCER1, FCER1A, FCER2, FGFR, FLAP, FOLH1, Gi24, GITR, GITRL, GPR5, GP100, GPRC5D, HER2, HER3, ICOSL, ICOS, HHLA2, HMGB1, HVEM, IDO, IFNa, IgE, IGF1R, IL2Rbeta, IL1, ILIA, IL1B, IL1F10, IL2, IL4, IL4Ra, IL5, IL5R, IL6, IL7, IL7Ra, IL8, IL9, IL9R, IL10, rhILlO, IL12, IL13, IL13Ra1, IL13Ra2, IL15, IL17, IL17Rb, IL18, IL22, IL23, IL25, IL7, IL33, IL35, ITGB4, ITK, KIR, LAG3, LAMP1, leptin, LPFS2, MHC class II, MUC-1, MUC-16, NCR3LG1, NKG2D, NKp46, NTPDase-1, OX40, OX40L, PD-1, PD-L1, PD-L2, PROM1, S152, SIRPalpha, SISP1, SLC, SPG64, ST2, STEAP1, STEAP2, Syk kinase, STEAP1, TROP2, TACI, TDO, TGFBETA, T14, TIGIT, TIM3, TLR, TLR2, TLR4, TLR5, TLR9, TMEF1, TNFa, TNFRSF7, Tp55, TREM1, TSLP, TSLPR, TWEAK, VEGF, VISTA, Vstm3, or WUCAM, or combinations thereof. For example, the VH1 may specifically bind to CD3. For example, the VH1 may specifically bind to Trop2. For example, the VH1 may specifically bind to cMet. For example, the VH1 may specifically bind to CD19. For example, the VH1 may specifically bind to CD20. In some aspects, the antigen binding polypeptide complex comprises a VH2 that specifically binds to A2AR, APRIL, ATPDase, BAFF, BAFFR, BCMA, BlyS, BTK, BTLA, B7DC, B7H1, B7H2, B7H3, B7H4, B7H5, B7H6, B7H7, B7RP1, B7-4, C3, C5, CCL2, CCL3, CCL4, CCL5, CCL7, CCL8, CCL11, CCL15, CCL17, CCL19, CCL20, CCL21, CCL25, CCR3, CCR4, CD16A, CD19, CD20, CD24, CD27, CD28, CD30, CD38, CD39, CD40, CD40L, CD47, CD52, CD70, CD80, CD86, CD123, CD133, CD137, CD137L, CD160, CD272, CEACAM5, CLEC9, CLEC91, CRTH2, CSF-1, CSF-2, CSF-3, CXCL1, CXCL2, CXCL4, CXCL12, CXCL13, CXCR3, cMet, CTLA4, DLL3, DLL4, DNGR-1, E-cadherin, EGFR, ENTPD1, EpCAM, FCER1, FCER1A, FCER2, FGFR, FLAP, FOLH1, Gi24, GITR, GITRL, GPR5, GP100, GPRC5D, HER2, HER3, ICOSL, ICOS, HHLA2, HMGB1, HVEM, IDO, IFNa, IgE, IGF1R, IL2Rbeta, IL1, ILIA, IL1B, IL1F10, IL2, IL4, IL4Ra, IL5, IL5R, IL6, IL7, IL7Ra, IL8, IL9, IL9R, IL10, rhILlO, IL12, IL13, IL13Ra1, IL13Ra2, IL15, IL17, IL17Rb, IL18, IL22, IL23, IL25, IL7, IL33, IL35, ITGB4, ITK, KIR, LAG3, LAMP1, leptin, LPFS2, MHC class II, MUC-1, MUC-16, NCR3LG1, NKG2D, NKp46, NTPDase-1, OX40, OX40L, PD-1, PD-L1, PD-L2, PROM1, S152, SIRPalpha, SISP1, SLC, SPG64, ST2, STEAP1, STEAP2, Syk kinase, STEAP1, TROP2, TACI, TDO, TGFBETA, T14, TIGIT, TIM3, TLR, TLR2, TLR4, TLR5, TLR9, TMEF1, TNFa, TNFRSF7, Tp55, TREM1, TSLP, TSLPR, TWEAK, VEGF, VISTA, Vstm3, or WUCAM, or combinations thereof. For example, the VH2 may specifically bind to CD3. For example, the VH2 may specifically bind to Trop2. For example, the VH2 may specifically bind to cMet. For example, the VH2 may specifically bind to CD19. For example, the VH2 may specifically bind to CD20. In some aspects, the antigen binding polypeptide complex comprises a VH3 that specifically binds to A2AR, APRIL, ATPDase, BAFF, BAFFR, BCMA, BlyS, BTK, BTLA, B7DC, B7H1, B7H2, B7H3, B7H4, B7H5, B7H6, B7H7, B7RP1, B7-4, C3, C5, CCL2, CCL3, CCL4, CCL5, CCL7, CCL8, CCL11, CCL15, CCL17, CCL19, CCL20, CCL21, CCL25, CCR3, CCR4, CD16A, CD19, CD20, CD24, CD27, CD28, CD30, CD38, CD39, CD40, CD40L, CD47, CD52, CD70, CD80, CD86, CD123, CD133, CD137, CD137L, CD160, CD272, CEACAM5, CLEC9, CLEC91, CRTH2, CSF-1, CSF-2, CSF-3, CXCL1, CXCL2, CXCL4, CXCL12, CXCL13, CXCR3, cMet, CTLA4, DLL3, DLL4, DNGR-1, E-cadherin, EGFR, ENTPD1, EpCAM, FCER1, FCER1A, FCER2, FGFR, FLAP, FOLH1, Gi24, GITR, GITRL, GPR5, GP100, GPRC5D, HER2, HER3, ICOSL, ICOS, HHLA2, HMGB1, HVEM, IDO, IFNa, IgE, IGF1R, IL2Rbeta, IL1, ILIA, IL1B, IL1F10, IL2, IL4, IL4Ra, IL5, IL5R, IL6, IL7, IL7Ra, IL8, IL9, IL9R, IL10, rhILlO, IL12, IL13, IL13Ra1, IL13Ra2, IL15, IL17, IL17Rb, IL18, IL22, IL23, IL25, IL7, IL33, IL35, ITGB4, ITK, KIR, LAG3, LAMP1, leptin, LPFS2, MHC class II, MUC-1, MUC-16, NCR3LG1, NKG2D, NKp46, NTPDase-1, OX40, OX40L, PD-1, PD-L1, PD-L2, PROM1, S152, SIRPalpha, SISP1, SLC, SPG64, ST2, STEAP1, STEAP2, Syk kinase, STEAP1, TROP2, TACI, TDO, TGFBETA, T14, TIGIT, TIM3, TLR, TLR2, TLR4, TLR5, TLR9, TMEF1, TNFa, TNFRSF7, Tp55, TREM1, TSLP, TSLPR, TWEAK, VEGF, VISTA, Vstm3, or WUCAM, or combinations thereof. For example, the VH3 may specifically bind to CD3. For example, the VH3 may specifically bind to Trop2. For example, the VH3 may specifically bind to cMet. For example, the VH3 may specifically bind to CD19. For example, the VH3 may specifically bind to CD20. In some aspects, the antigen binding polypeptide complex comprises a VH4 that specifically binds to A2AR, APRIL, ATPDase, BAFF, BAFFR, BCMA, BlyS, BTK, BTLA, B7DC, B7H1, B7H2, B7H3, B7H4, B7H5, B7H6, B7H7, B7RP1, B7-4, C3, C5, CCL2, CCL3, CCL4, CCL5, CCL7, CCL8, CCL11, CCL15, CCL17, CCL19, CCL20, CCL21, CCL25, CCR3, CCR4, CD16A, CD19, CD20, CD24, CD27, CD28, CD30, CD38, CD39, CD40, CD40L, CD47, CD52, CD70, CD80, CD86, CD123, CD133, CD137, CD137L, CD160, CD272, CEACAM5, CLEC9, CLEC91, CRTH2, CSF-1, CSF-2, CSF-3, CXCL1, CXCL2, CXCL4, CXCL12, CXCL13, CXCR3, cMet, CTLA4, DLL3, DLL4, DNGR-1, E-cadherin, EGFR, ENTPD1, EpCAM, FCER1, FCER1A, FCER2, FGFR, FLAP, FOLH1, Gi24, GITR, GITRL, GPR5, GP100, GPRC5D, HER2, HER3, ICOSL, ICOS, HHLA2, HMGB1, HVEM, IDO, IFNa, IgE, IGF1R, IL2Rbeta, IL1, ILIA, IL1B, IL1F10, IL2, IL4, IL4Ra, IL5, IL5R, IL6, IL7, IL7Ra, IL8, IL9, IL9R, IL10, rhILlO, IL12, IL13, IL13Ra1, IL13Ra2, IL15, IL17, IL17Rb, IL18, IL22, IL23, IL25, IL7, IL33, IL35, ITGB4, ITK, KIR, LAG3, LAMP1, leptin, LPFS2, MHC class II, MUC-1, MUC-16, NCR3LG1, NKG2D, NKp46, NTPDase-1, OX40, OX40L, PD-1, PD-L1, PD-L2, PROM1, S152, SIRPalpha, SISP1, SLC, SPG64, ST2, STEAP1, STEAP2, Syk kinase, STEAP1, TROP2, TACI, TDO, TGFBETA, T14, TIGIT, TIM3, TLR, TLR2, TLR4, TLR5, TLR9, TMEF1, TNFa, TNFRSF7, Tp55, TREM1, TSLP, TSLPR, TWEAK, VEGF, VISTA, Vstm3, or WUCAM, or combinations thereof. For example, the VH4 may specifically bind to CD3. For example, the VH4 may specifically bind to Trop2. For example, the VH4 may specifically bind to cMet. For example, the VH4 may specifically bind to CD19. For example, the VH4 may specifically bind to CD20. In some aspects, the antigen binding polypeptide comprises a VH1 and VL1 that specifically binds to cMet, a VH2 and VL2 that specifically binds to Trop2, a VH3 and VL3 that specifically binds to CD28, and a VH4 and VL4 that specifically binds to CD3. In some aspects, the antigen binding polypeptide comprises a VH1 and VL1 that specifically binds to Trop2, a VH2 and VL2 that specifically binds to cMet, a VH3 and VL3 that specifically binds to CD28, and a VH4 and VL4 that specifically binds to CD3. In some aspects, the antigen binding polypeptide comprises a VH1 and VL1 that specifically binds to cMet, a VH2 and VL2 that specifically binds to CD28, a VH3 and VL3 that specifically binds to Trop2, and a VH4 and VL4 that specifically binds to CD3. In some aspects, the antigen binding polypeptide comprises a VH1 and VL1 that specifically binds to Trop2, a VH2 and VL2 that specifically binds to CD28, a VH3 and VL3 that specifically binds to cMet, and a VH4 and VL4 that specifically binds to CD3. In some aspects, the antigen binding polypeptide comprises a VH1 and VL1 that specifically binds to CD28, a VH2 and VL2 that specifically binds to cMet, a VH3 and VL3 that specifically binds to Trop2, and VH4 and VL4 that specifically binds to CD3. In some aspects, the antigen binding polypeptide comprises a VH1 and VL1 that specifically binds to CD28, a VH2 and VL2 that specifically binds to Trop2, a VH3 and VL3 that specifically binds to cMet, and VH4 and VL4 that specifically binds to CD3. In some aspects, the antigen binding polypeptide comprises a VH1 and VL1 that specifically binds to CD19, a VH2 and VL2 that specifically binds to CD20, a VH3 and VL3 that specifically binds to CD28, and VH4 and VL4 that specifically binds to CD3. In some aspects, the antigen binding polypeptide comprises a VH1 and VL1 that specifically binds to CD20, a VH2 and VL2 that specifically binds to CD19, a VH3 and VL3 that specifically binds to CD28, and VH4 and VL4 that specifically binds to CD3. In some aspects, the antigen binding polypeptide comprises a VH1 and VL1 that specifically binds to CD19, a VH2 and VL2 that specifically binds to CD28, a VH3 and VL3 that specifically binds to CD20, and VH4 and VL4 that specifically binds to CD3. In some aspects, the antigen binding polypeptide comprises a VH1 and VL1 that specifically binds to CD20, a VH2 and VL2 that specifically binds to CD28, a VH3 and VL3 that specifically binds to CD19, and VH4 and VL4 that specifically binds to CD3. In some aspects, the antigen binding polypeptide comprises a VH1 and VL1 that specifically binds to CD28, a VH2 and VL2 that specifically binds to CD19, a VH3 and VL3 that specifically binds to CD20, and VH4 and VL4 that specifically binds to CD3. In some aspects, the antigen binding polypeptide comprises a VH1 and VL1 that specifically binds to CD28, a VH2 and VL2 that specifically binds to CD20, a VH3 and VL3 that specifically binds to CD19, and VH4 and VL4 that specifically binds to CD3. For the avoidance of doubt, all the antigen binding polypeptide complex structures described herein can be combined with any one or more of the targets described herein. Any and all disclosure herein in relation to targets for antigen binding polypeptide complexes of the invention is generally applicable, and applies equally and without reservation to each and every antigen binding polypeptide complex described herein. The VL1, VL2, VL3, VL4, VH1, VH2, VH3, and/or VH4 of each and every antigen binding polypeptide complex described herein may independently bind to any one of said particularly preferred targets.

In some aspects, an antigen binding polypeptide complex of the invention comprises a VL that specifically binds to a TAA, the VL having a CDR1 comprising an amino acid sequence having at least 90% identity, at least 95% identity, or 100% identity to any one of SEQ ID NOs:67, 75, 83, 91, 107, 115, 123, 131, 385, 405, 415, 441, 452, 462, 472, 655, 691, 727, 393, 486, 504, 536, 548, 753, 789, 825, 871, 879, 490, 496, 745, 781, 817, 645, 681, 771, 843, 621, 637, 673, 709 and 871; a CDR2 comprising an amino acid sequence having at least 90% identity, at least 95% identity, or 100% identity to any one of SEQ ID NOs:68, 76, 84, 92, 108, 116, 124, 132, 386 (YTS); 406 (YTS), 416 (YTS), 442 (YTS), 453 (YTS), 463 (YTS), 473 (YTS), 656, 692, 728, 394, 487 (AT), 505 (AT), 537, 549, 754, 790, 826, 872, 880, 491 (DA), 497 (DA), 746, 782, 818, 646, 682, 772, 844, 622, 638, 674, 710 and 872; and a CDR3 comprising an amino acid sequence having at least 90% identity, at least 95% identity, or 100% identity to any one of SEQ ID NOs:69, 77, 85, 93, 109, 117, 125, 133, 387, 407, 417, 443, 454, 464, 474, 657, 693, 729, 395, 488, 506, 538, 550, 755, 791, 827, 873, 881, 492, 498, 747, 783, 819, 647, 683, 773, 845, 623, 639, 675, 711 and 873; and a VH that specifically binds to a TAA, the VH having a CDR1 comprising an amino acid sequence having at least 90% identity, at least 95% identity, or 100% identity to any one of SEQ ID NOs:64, 72, 80, 88, 104, 112, 120, 128, 389, 409, 419, 429, 659, 695, 731, 397, 508, 540, 552, 757, 793, 829, 875, 500, 749, 785, 821, 649, 685, 775, 847, 625, 641, 677, 713 and 867; a CDR2 comprising an amino acid sequence having at least 90% identity, at least 95% identity, or 100% identity to any one of SEQ ID NOs:65, 73, 81, 89, 105, 113, 121, 129, 390, 410, 420, 430, 660, 696, 732, 398, 509, 541, 553, 758, 794, 830, 876, 501, 750, 786, 822, 650, 686, 776, 848, 626, 642, 678, 714 and 868; and a CDR3 comprising an amino acid sequence having at least 90% identity, at least 95% identity, or 100% identity to any one of SEQ ID NOs:66, 74, 82, 90, 106, 114, 122, 130, 391, 411, 421, 431, 661, 697, 733, 399, 510, 542, 554, 759, 795, 831, 877, 502, 751, 787, 823, 651, 687, 777, 849, 627, 643, 679, 715 and 869.

43) In some aspects, the VL that specifically binds to CD38 comprises a CDR1 comprising an amino acid sequence having at least 90% identity to SEQ ID NO: 67; a CDR2 comprising an amino acid sequence having at least 90% identity to SEQ ID NO:68; and a CDR3 comprising an amino acid sequence having at least 90% identity to SEQ ID NO:69; and the VH that specifically binds to CD38 comprises a CDR1 comprising an amino acid sequence having at least 90% identity to SEQ ID NO:64; a CDR2 comprising an amino acid sequence having at least 90% identity to SEQ ID NO:65; and a CDR3 comprising an amino acid sequence having at least 90% identity to SEQ ID NO:66. In some aspects, the VL that specifically binds to cMet comprises a CDR1 comprising an amino acid sequence having at least 90% identity to SEQ ID NO:75; a CDR2 comprising an amino acid sequence having at least 90% identity to SEQ ID NO:76; and a CDR3 comprising an amino acid sequence having at least 90% identity to SEQ ID NO:77; and the VH that specifically binds to cMet comprises a CDR1 comprising an amino acid sequence having at least 90% identity to SEQ ID NO:72; a CDR2 comprising an amino acid sequence having at least 90% identity to SEQ ID NO:73; and a CDR3 comprising an amino acid sequence having at least 90% identity to SEQ ID NO:74. In some aspects, the VL that specifically binds to Trop2 comprises a CDR1 comprising an amino acid sequence having at least 90% identity to SEQ ID NO:83; a CDR2 comprising an amino acid sequence having at least 90% identity to SEQ ID NO:84; and a CDR3 comprising an amino acid sequence having at least 90% identity to SEQ ID NO:85; and the VH that specifically binds to Trop2 comprises a CDR1 comprising an amino acid sequence having at least 90% identity to SEQ ID NO:80; a CDR2 comprising an amino acid sequence having at least 90% identity to SEQ ID NO:81; and a CDR3 comprising an amino acid sequence having at least 90% identity to SEQ ID NO:82. In some aspects, the VL that specifically binds to Trop2 comprises a CDR1 comprising an amino acid sequence having at least 90% identity to SEQ ID NO:91; a CDR2 comprising an amino acid sequence having at least 90% identity to SEQ ID NO:92; and a CDR3 comprising an amino acid sequence having at least 90% identity to SEQ ID NO:93; and the VH that specifically binds to Trop2 comprises a CDR1 comprising an amino acid sequence having at least 90% identity to SEQ ID NO:88; a CDR2 comprising an amino acid sequence having at least 90% identity to SEQ ID NO:89; and a CDR3 comprising an amino acid sequence having at least 90% identity to SEQ ID NO:90. In some aspects, the VL that specifically binds to CD3 comprises a CDR1 comprising an amino acid sequence having at least 90% identity to SEQ ID NO:99; a CDR2 comprising an amino acid sequence having at least 90% identity to SEQ ID NO: 100; and a CDR3 comprising an amino acid sequence having at least 90% identity to SEQ ID NO:101; and the VH that specifically binds to CD3 comprises a CDR1 comprising an amino acid sequence having at least 90% identity to SEQ ID NO:96; a CDR2 comprising an amino acid sequence having at least 90% identity to SEQ ID NO:97; and a CDR3 comprising an amino acid sequence having at least 90% identity to SEQ ID NO:98. In some aspects, the VL that specifically binds to CD20 comprises a CDR1 comprising an amino acid sequence having at least 90% identity to SEQ ID NO: 107 a CDR2 comprising an amino acid sequence having at least 90% identity to SEQ ID NO:108; and a CDR3 comprising an amino acid sequence having at least 90% identity to SEQ ID NO:109; and the VH that specifically binds to CD20 comprises a CDR1 comprising an amino acid sequence having at least 90% identity to SEQ ID NO:104; a CDR2 comprising an amino acid sequence having at least 90% identity to SEQ ID NO:105; and a CDR3 comprising an amino acid sequence having at least 90% identity to SEQ ID NO:106. In some aspects, the VL that specifically binds to CD20 comprises a CDR1 comprising an amino acid sequence having at least 90% identity to SEQ ID NO:115; a CDR2 comprising an amino acid sequence having at least 90% identity to SEQ ID NO: 116; and a CDR3 comprising an amino acid sequence having at least 90% identity to SEQ ID NO: 117; and the VH that specifically binds to CD20 comprises a CDR1 comprising an amino acid sequence having at least 90% identity to SEQ ID NO:112; a CDR2 comprising an amino acid sequence having at least 90% identity to SEQ ID NO:113; and a CDR3 comprising an amino acid sequence having at least 90% identity to SEQ ID NO:114. In some aspects, the VL that specifically binds to CD19 comprises a CDR1 comprising an amino acid sequence having at least 90% identity to SEQ ID NO:123; a CDR2 comprising an amino acid sequence having at least 90% identity to SEQ ID NO: 124; and a CDR3 comprising an amino acid sequence having at least 90% identity to SEQ ID NO:125; and the VH that specifically binds to CD19 comprises a CDR1 comprising an amino acid sequence having at least 90% identity to SEQ ID NO:120; a CDR2 comprising an amino acid sequence having at least 90% identity to SEQ ID NO:121; and a CDR3 comprising an amino acid sequence having at least 90% identity to SEQ ID NO:122. In some aspects, the VL that specifically binds to CD19 comprises a CDR1 comprising an amino acid sequence having at least 90% identity to SEQ ID NO:131; a CDR2 comprising an amino acid sequence having at least 90% identity to SEQ ID NO: 132; and a CDR3 comprising an amino acid sequence having at least 90% identity to SEQ ID NO:133; and the VH that specifically binds to CD19 comprises a CDR1 comprising an amino acid sequence having at least 90% identity to SEQ ID NO:128; a CDR2 comprising an amino acid sequence having at least 90% identity to SEQ ID NO: 129; and a CDR3 comprising an amino acid sequence having at least 90% identity to SEQ ID NO:130. In some aspects, the VL that specifically binds to Trop2 comprises a CDR1 comprising an amino acid sequence having at least 90% identity to SEQ ID NO: 385; a CDR2 comprising an amino acid sequence having at least 90% identity to SEQ ID NO:386 (YTS); and a CDR3 comprising an amino acid sequence having at least 90% identity to SEQ ID NO:387; and the VH that specifically binds to Trop2 comprises a CDR1 comprising an amino acid sequence having at least 90% identity to SEQ ID NO:389; a CDR2 comprising an amino acid sequence having at least 90% identity to SEQ ID NO:390; and a CDR3 comprising an amino acid sequence having at least 90% identity to SEQ ID NO:391. In some aspects, the VL that specifically binds to Trop2 comprises a CDR1 comprising an amino acid sequence having at least 90% identity to SEQ ID NO: 405; a CDR2 comprising an amino acid sequence having at least 90% identity to SEQ ID NO:406 (YTS); and a CDR3 comprising an amino acid sequence having at least 90% identity to SEQ ID NO:407; and the VH that specifically binds to Trop2 comprises a CDR1 comprising an amino acid sequence having at least 90% identity to SEQ ID NO:409; a CDR2 comprising an amino acid sequence having at least 90% identity to SEQ ID NO:410; and a CDR3 comprising an amino acid sequence having at least 90% identity to SEQ ID NO:411. In some aspects, the VL that specifically binds to Trop2 comprises a CDR1 comprising an amino acid sequence having at least 90% identity to SEQ ID NO: 415; a CDR2 comprising an amino acid sequence having at least 90% identity to SEQ ID NO:416 (YTS); and a CDR3 comprising an amino acid sequence having at least 90% identity to SEQ ID NO:417; and the VH that specifically binds to Trop2 comprises a CDR1 comprising an amino acid sequence having at least 90% identity to SEQ ID NO:419; a CDR2 comprising an amino acid sequence having at least 90% identity to SEQ ID NO:420; and a CDR3 comprising an amino acid sequence having at least 90% identity to SEQ ID NO:421. In some aspects, the VL that specifically binds to Trop2 comprises a CDR1 comprising an amino acid sequence having at least 90% identity to SEQ ID NO: 441; a CDR2 comprising an amino acid sequence having at least 90% identity to SEQ ID NO:442 (YTS); and a CDR3 comprising an amino acid sequence having at least 90% identity to SEQ ID NO:443; and the VH that specifically binds to Trop2 comprises a CDR1 comprising an amino acid sequence having at least 90% identity to SEQ ID NO:429; a CDR2 comprising an amino acid sequence having at least 90% identity to SEQ ID NO:430; and a CDR3 comprising an amino acid sequence having at least 90% identity to SEQ ID NO:431. In some aspects, the VL that specifically binds to Trop2 comprises a CDR1 comprising an amino acid sequence having at least 90% identity to SEQ ID NO: 452; a CDR2 comprising an amino acid sequence having at least 90% identity to SEQ ID NO:453 (YTS); and a CDR3 comprising an amino acid sequence having at least 90% identity to SEQ ID NO:454; and the VH that specifically binds to Trop2 comprises a CDR1 comprising an amino acid sequence having at least 90% identity to SEQ ID NO:429; a CDR2 comprising an amino acid sequence having at least 90% identity to SEQ ID NO:430; and a CDR3 comprising an amino acid sequence having at least 90% identity to SEQ ID NO:431. In some aspects, the VL that specifically binds to Trop2 comprises a CDR1 comprising an amino acid sequence having at least 90% identity to SEQ ID NO: 462; a CDR2 comprising an amino acid sequence having at least 90% identity to SEQ ID NO:463 (YTS); and a CDR3 comprising an amino acid sequence having at least 90% identity to SEQ ID NO:464; and the VH that specifically binds to Trop2 comprises a CDR1 comprising an amino acid sequence having at least 90% identity to SEQ ID NO:429; a CDR2 comprising an amino acid sequence having at least 90% identity to SEQ ID NO:430; and a CDR3 comprising an amino acid sequence having at least 90% identity to SEQ ID NO:431. In some aspects, the VL that specifically binds to Trop2 comprises a CDR1 comprising an amino acid sequence having at least 90% identity to SEQ ID NO: 472; a CDR2 comprising an amino acid sequence having at least 90% identity to SEQ ID NO:473 (YTS); and a CDR3 comprising an amino acid sequence having at least 90% identity to SEQ ID NO:474; and the VH that specifically binds to Trop2 comprises a CDR1 comprising an amino acid sequence having at least 90% identity to SEQ ID NO:429; a CDR2 comprising an amino acid sequence having at least 90% identity to SEQ ID NO:430; and a CDR3 comprising an amino acid sequence having at least 90% identity to SEQ ID NO:431. In some aspects, the VL that specifically binds to Trop2 comprises a CDR1 comprising an amino acid sequence having at least 90% identity to SEQ ID NO: 655; a CDR2 comprising an amino acid sequence having at least 90% identity to SEQ ID NO:656; and a CDR3 comprising an amino acid sequence having at least 90% identity to SEQ ID NO:657; and the VH that specifically binds to Trop2 comprises a CDR1 comprising an amino acid sequence having at least 90% identity to SEQ ID NO:659; a CDR2 comprising an amino acid sequence having at least 90% identity to SEQ ID NO:660; and a CDR3 comprising an amino acid sequence having at least 90% identity to SEQ ID NO:661. In some aspects, the VL that specifically binds to Trop2 comprises a CDR1 comprising an amino acid sequence having at least 90% identity to SEQ ID NO: 691; a CDR2 comprising an amino acid sequence having at least 90% identity to SEQ ID NO:692; and a CDR3 comprising an amino acid sequence having at least 90% identity to SEQ ID NO:693; and the VH that specifically binds to Trop2 comprises a CDR1 comprising an amino acid sequence having at least 90% identity to SEQ ID NO:695; a CDR2 comprising an amino acid sequence having at least 90% identity to SEQ ID NO:696; and a CDR3 comprising an amino acid sequence having at least 90% identity to SEQ ID NO:697. In some aspects, the VL that specifically binds to Trop2 comprises a CDR1 comprising an amino acid sequence having at least 90% identity to SEQ ID NO: 727; a CDR2 comprising an amino acid sequence having at least 90% identity to SEQ ID NO:728; and a CDR3 comprising an amino acid sequence having at least 90% identity to SEQ ID NO:729; and the VH that specifically binds to Trop2 comprises a CDR1 comprising an amino acid sequence having at least 90% identity to SEQ ID NO:731; a CDR2 comprising an amino acid sequence having at least 90% identity to SEQ ID NO:732; and a CDR3 comprising an amino acid sequence having at least 90% identity to SEQ ID NO:733. In some aspects, the VL that specifically binds to HER2 comprises a CDR1 comprising an amino acid sequence having at least 90% identity to SEQ ID NO: 393; a CDR2 comprising an amino acid sequence having at least 90% identity to SEQ ID NO:394; and a CDR3 comprising an amino acid sequence having at least 90% identity to SEQ ID NO:395; and the VH that specifically binds to HER2 comprises a CDR1 comprising an amino acid sequence having at least 90% identity to SEQ ID NO:397; a CDR2 comprising an amino acid sequence having at least 90% identity to SEQ ID NO:398; and a CDR3 comprising an amino acid sequence having at least 90% identity to SEQ ID NO:399. In some aspects, the VL that specifically binds to CD20 comprises a CDR1 comprising an amino acid sequence having at least 90% identity to SEQ ID NO: 486; a CDR2 comprising an amino acid sequence having at least 90% identity to SEQ ID NO:487 (AT); and a CDR3 comprising an amino acid sequence having at least 90% identity to SEQ ID NO:488; and the VH that specifically binds to CD20 comprises a CDR1 comprising an amino acid sequence having at least 90% identity to SEQ ID NO:508; a CDR2 comprising an amino acid sequence having at least 90% identity to SEQ ID NO:509; and a CDR3 comprising an amino acid sequence having at least 90% identity to SEQ ID NO:510. In some aspects, the VL that specifically binds to CD20 comprises a CDR1 comprising an amino acid sequence having at least 90% identity to SEQ ID NO: 504; a CDR2 comprising an amino acid sequence having at least 90% identity to SEQ ID NO:505 (AT); and a CDR3 comprising an amino acid sequence having at least 90% identity to SEQ ID NO:506; and the VH that specifically binds to CD20 comprises a CDR1 comprising an amino acid sequence having at least 90% identity to SEQ ID NO:508; a CDR2 comprising an amino acid sequence having at least 90% identity to SEQ ID NO:509; and a CDR3 comprising an amino acid sequence having at least 90% identity to SEQ ID NO:510. In some aspects, the VL that specifically binds to CD20 comprises a CDR1 comprising an amino acid sequence having at least 90% identity to SEQ ID NO: 536; a CDR2 comprising an amino acid sequence having at least 90% identity to SEQ ID NO:537; and a CDR3 comprising an amino acid sequence having at least 90% identity to SEQ ID NO:538; and the VH that specifically binds to CD20 comprises a CDR1 comprising an amino acid sequence having at least 90% identity to SEQ ID NO:540; a CDR2 comprising an amino acid sequence having at least 90% identity to SEQ ID NO:541; and a CDR3 comprising an amino acid sequence having at least 90% identity to SEQ ID NO:542. In some aspects, the VL that specifically binds to CD20 comprises a CDR1 comprising an amino acid sequence having at least 90% identity to SEQ ID NO:548; a CDR2 comprising an amino acid sequence having at least 90% identity to SEQ ID NO:549; and a CDR3 comprising an amino acid sequence having at least 90% identity to SEQ ID NO:550; and the VH that specifically binds to CD20 comprises a CDR1 comprising an amino acid sequence having at least 90% identity to SEQ ID NO:552; a CDR2 comprising an amino acid sequence having at least 90% identity to SEQ ID NO:553; and a CDR3 comprising an amino acid sequence having at least 90% identity to SEQ ID NO:554. In some aspects, the VL that specifically binds to CD20 comprises a CDR1 comprising an amino acid sequence having at least 90% identity to SEQ ID NO: 753; a CDR2 comprising an amino acid sequence having at least 90% identity to SEQ ID NO:754; and a CDR3 comprising an amino acid sequence having at least 90% identity to SEQ ID NO:755; and the VH that specifically binds to CD20 comprises a CDR1 comprising an amino acid sequence having at least 90% identity to SEQ ID NO:757; a CDR2 comprising an amino acid sequence having at least 90% identity to SEQ ID NO:758; and a CDR3 comprising an amino acid sequence having at least 90% identity to SEQ ID NO:759. In some aspects, the VL that specifically binds to CD20 comprises a CDR1 comprising an amino acid sequence having at least 90% identity to SEQ ID NO: 789; a CDR2 comprising an amino acid sequence having at least 90% identity to SEQ ID NO:790; and a CDR3 comprising an amino acid sequence having at least 90% identity to SEQ ID NO:791; and the VH that specifically binds to CD20 comprises a CDR1 comprising an amino acid sequence having at least 90% identity to SEQ ID NO:793; a CDR2 comprising an amino acid sequence having at least 90% identity to SEQ ID NO:794; and a CDR3 comprising an amino acid sequence having at least 90% identity to SEQ ID NO:795. In some aspects, the VL that specifically binds to CD20 comprises a CDR1 comprising an amino acid sequence having at least 90% identity to SEQ ID NO: 825; a CDR2 comprising an amino acid sequence having at least 90% identity to SEQ ID NO: 826; and a CDR3 comprising an amino acid sequence having at least 90% identity to SEQ ID NO: 827; and the VH that specifically binds to CD20 comprises a CDR1 comprising an amino acid sequence having at least 90% identity to SEQ ID NO:829; a CDR2 comprising an amino acid sequence having at least 90% identity to SEQ ID NO:830; and a CDR3 comprising an amino acid sequence having at least 90% identity to SEQ ID NO:831. In some aspects, the VL that specifically binds to CD20 comprises a CDR1 comprising an amino acid sequence having at least 90% identity to SEQ ID NO: 871; a CDR2 comprising an amino acid sequence having at least 90% identity to SEQ ID NO:872; and a CDR3 comprising an amino acid sequence having at least 90% identity to SEQ ID NO:873; and the VH that specifically binds to CD20 comprises a CDR1 comprising an amino acid sequence having at least 90% identity to SEQ ID NO:; a CDR2 comprising an amino acid sequence having at least 90% identity to SEQ ID NO:; and a CDR3 comprising an amino acid sequence having at least 90% identity to SEQ ID NO:. In some aspects, the VL that specifically binds to CD20 comprises a CDR1 comprising an amino acid sequence having at least 90% identity to SEQ ID NO: 879; a CDR2 comprising an amino acid sequence having at least 90% identity to SEQ ID NO:880; and a CDR3 comprising an amino acid sequence having at least 90% identity to SEQ ID NO:881; and the VH that specifically binds to CD20 comprises a CDR1 comprising an amino acid sequence having at least 90% identity to SEQ ID NO:875; a CDR2 comprising an amino acid sequence having at least 90% identity to SEQ ID NO:876; and a CDR3 comprising an amino acid sequence having at least 90% identity to SEQ ID NO:877. In some aspects, the VL that specifically binds to CD19 comprises a CDR1 comprising an amino acid sequence having at least 90% identity to SEQ ID NO: 490; a CDR2 comprising an amino acid sequence having at least 90% identity to SEQ ID NO:491 (AT); and a CDR3 comprising an amino acid sequence having at least 90% identity to SEQ ID NO:492; and the VH that specifically binds to CD19 comprises a CDR1 comprising an amino acid sequence having at least 90% identity to SEQ ID NO:500; a CDR2 comprising an amino acid sequence having at least 90% identity to SEQ ID NO:501; and a CDR3 comprising an amino acid sequence having at least 90% identity to SEQ ID NO:502. In some aspects, the VL that specifically binds to CD19 comprises a CDR1 comprising an amino acid sequence having at least 90% identity to SEQ ID NO: 496; a CDR2 comprising an amino acid sequence having at least 90% identity to SEQ ID NO:497 (AT); and a CDR3 comprising an amino acid sequence having at least 90% identity to SEQ ID NO:498; and the VH that specifically binds to CD19 comprises a CDR1 comprising an amino acid sequence having at least 90% identity to SEQ ID NO:500; a CDR2 comprising an amino acid sequence having at least 90% identity to SEQ ID NO:501; and a CDR3 comprising an amino acid sequence having at least 90% identity to SEQ ID NO:502. In some aspects, the VL that specifically binds to CD19 comprises a CDR1 comprising an amino acid sequence having at least 90% identity to SEQ ID NO: 745; a CDR2 comprising an amino acid sequence having at least 90% identity to SEQ ID NO: 746; and a CDR3 comprising an amino acid sequence having at least 90% identity to SEQ ID NO:747; and the VH that specifically binds to CD19 comprises a CDR1 comprising an amino acid sequence having at least 90% identity to SEQ ID NO:749; a CDR2 comprising an amino acid sequence having at least 90% identity to SEQ ID NO:750; and a CDR3 comprising an amino acid sequence having at least 90% identity to SEQ ID NO:751. In some aspects, the VL that specifically binds to CD19 comprises a CDR1 comprising an amino acid sequence having at least 90% identity to SEQ ID NO: 781; a CDR2 comprising an amino acid sequence having at least 90% identity to SEQ ID NO: 782; and a CDR3 comprising an amino acid sequence having at least 90% identity to SEQ ID NO:783; and the VH that specifically binds to CD19 comprises a CDR1 comprising an amino acid sequence having at least 90% identity to SEQ ID NO:785; a CDR2 comprising an amino acid sequence having at least 90% identity to SEQ ID NO:786; and a CDR3 comprising an amino acid sequence having at least 90% identity to SEQ ID NO:787. In some aspects, the VL that specifically binds to CD19 comprises a CDR1 comprising an amino acid sequence having at least 90% identity to SEQ ID NO: 817; a CDR2 comprising an amino acid sequence having at least 90% identity to SEQ ID NO:818; and a CDR3 comprising an amino acid sequence having at least 90% identity to SEQ ID NO:819; and the VH that specifically binds to CD19 comprises a CDR1 comprising an amino acid sequence having at least 90% identity to SEQ ID NO:821; a CDR2 comprising an amino acid sequence having at least 90% identity to SEQ ID NO:822; and a CDR3 comprising an amino acid sequence having at least 90% identity to SEQ ID NO:823. In some aspects, the VL that specifically binds to CD28 comprises a CDR1 comprising an amino acid sequence having at least 90% identity to SEQ ID NO: 645; a CDR2 comprising an amino acid sequence having at least 90% identity to SEQ ID NO:646; and a CDR3 comprising an amino acid sequence having at least 900% identity to SEQ ID NO:647; and the VH that specifically binds to CD28 comprises a CDR1 comprising an amino acid sequence having at least 90% identity to SEQ ID NO:649; a CDR2 comprising an amino acid sequence having at least 90% identity to SEQ ID NO: 650; and a CDR3 comprising an amino acid sequence having at least 90% identity to SEQ ID NO:651. In some aspects, the VL that specifically binds to CD28 comprises a CDR1 comprising an amino acid sequence having at least 90% identity to SEQ ID NO: 681; a CDR2 comprising an amino acid sequence having at least 90% identity to SEQ ID NO:682; and a CDR3 comprising an amino acid sequence having at least 90% identity to SEQ ID NO:683; and the VH that specifically binds to CD28 comprises a CDR1 comprising an amino acid sequence having at least 90% identity to SEQ ID NO:685; a CDR2 comprising an amino acid sequence having at least 90% identity to SEQ ID NO:686; and a CDR3 comprising an amino acid sequence having at least 90% identity to SEQ ID NO: 687. In some aspects, the VL that specifically binds to CD28 comprises a CDR1 comprising an amino acid sequence having at least 90% identity to SEQ ID NO: 771; a CDR2 comprising an amino acid sequence having at least 90% identity to SEQ ID NO:772; and a CDR3 comprising an amino acid sequence having at least 90% identity to SEQ ID NO:773; and the VH that specifically binds to CD28 comprises a CDR1 comprising an amino acid sequence having at least 90% identity to SEQ ID NO:775; a CDR2 comprising an amino acid sequence having at least 90% identity to SEQ ID NO:776; and a CDR3 comprising an amino acid sequence having at least 90% identity to SEQ ID NO:777. In some aspects, the VL that specifically binds to CD28 comprises a CDR1 comprising an amino acid sequence having at least 90% identity to SEQ ID NO: 843; a CDR2 comprising an amino acid sequence having at least 90% identity to SEQ ID NO:844; and a CDR3 comprising an amino acid sequence having at least 90% identity to SEQ ID NO:845; and the VH that specifically binds to CD28 comprises a CDR1 comprising an amino acid sequence having at least 90% identity to SEQ ID NO:847; a CDR2 comprising an amino acid sequence having at least 90% identity to SEQ ID NO: 848; and a CDR3 comprising an amino acid sequence having at least 90% identity to SEQ ID NO:849. In some aspects, the VL that specifically binds to BCMA comprises a CDR1 comprising an amino acid sequence having at least 90% identity to SEQ ID NO: 621; a CDR2 comprising an amino acid sequence having at least 90% identity to SEQ ID NO:622; and a CDR3 comprising an amino acid sequence having at least 90% identity to SEQ ID NO:623; and the VH that specifically binds to BCMA comprises a CDR1 comprising an amino acid sequence having at least 90% identity to SEQ ID NO:625; a CDR2 comprising an amino acid sequence having at least 90% identity to SEQ TD NO:626, and a CDR3 comprising an amino acid sequence having at least 900% identity to SEQ ID NO:627. In some aspects, the VL that specifically binds to cMet comprises a CDR1 comprising an amino acid sequence having at least 90% identity to SEQ ID NO: 637; a CDR2 comprising an amino acid sequence having at least 90% identity to SEQ ID NO:638; and a CDR3 comprising an amino acid sequence having at least 90% identity to SEQ ID NO:639; and the VH that specifically binds to cMet comprises a CDR1 comprising an amino acid sequence having at least 90% identity to SEQ ID NO:641; a CDR2 comprising an amino acid sequence having at least 90% identity to SEQ ID NO:642; and a CDR3 comprising an amino acid sequence having at least 90% identity to SEQ ID NO:643. In some aspects, the VL that specifically binds to cMet comprises a CDR1 comprising an amino acid sequence having at least 90% identity to SEQ ID NO: 673; a CDR2 comprising an amino acid sequence having at least 90% identity to SEQ ID NO:674; and a CDR3 comprising an amino acid sequence having at least 90% identity to SEQ ID NO:675; and the VH that specifically binds to cMet comprises a CDR1 comprising an amino acid sequence having at least 90% identity to SEQ ID NO:677; a CDR2 comprising an amino acid sequence having at least 90% identity to SEQ ID NO:678; and a CDR3 comprising an amino acid sequence having at least 90% identity to SEQ ID NO: 679. In some aspects, the VL that specifically binds to cMet comprises a CDR1 comprising an amino acid sequence having at least 90% identity to SEQ ID NO: 709; a CDR2 comprising an amino acid sequence having at least 90% identity to SEQ ID NO:710; and a CDR3 comprising an amino acid sequence having at least 90% identity to SEQ ID NO:711; and the VH that specifically binds to cMet comprises a CDR1 comprising an amino acid sequence having at least 90% identity to SEQ ID NO:713; a CDR2 comprising an amino acid sequence having at least 90% identity to SEQ ID NO:714; and a CDR3 comprising an amino acid sequence having at least 90% identity to SEQ ID NO:715. In some aspects, the VL that specifically binds to CD20 comprises a CDR1 comprising an amino acid sequence having at least 90% identity to SEQ ID NO:871; a CDR2 comprising an amino acid sequence having at least 90% identity to SEQ ID NO:872; and a CDR3 comprising an amino acid sequence having at least 90% identity to SEQ ID NO:873; and the VH that specifically binds to CD20 comprises a CDR1 comprising an amino acid sequence having at least 90% identity to SEQ ID NO:867; a CDR2 comprising an amino acid sequence having at least 90% identity to SEQ ID NO:868; and a CDR3 comprising an amino acid sequence having at least 90% identity to SEQ ID NO:869. As used herein, “at least 90% identity” includes at least 91%, 92%, 93%, 94%, 95%, 96%, 97%. 98%, 99% and 100% identity to the recited reference sequence. In some aspects, the VL that specifically binds to CD38 comprises a CDR1 comprising the amino acid sequence of SEQ ID NO:67; a CDR2 comprising the amino acid sequence of SEQ ID NO:68; and a CDR3 comprising the amino acid sequence of SEQ ID NO:69; and the VH that specifically binds to CD38 comprises a CDR1 comprising the amino acid sequence of SEQ ID NO:64; a CDR2 comprising the amino acid sequence of SEQ ID NO:65; and a CDR3 comprising the amino acid sequence of SEQ ID NO:66. In some aspects, the VL that specifically binds to cMet comprises a CDR1 comprising the amino acid sequence of SEQ ID NO:75; a CDR2 comprising the amino acid sequence of SEQ ID NO:76; and a CDR3 comprising the amino acid sequence of SEQ ID NO:77; and the VH that specifically binds to cMet comprises a CDR1 comprising the amino acid sequence of SEQ ID NO:72; a CDR2 comprising the amino acid sequence of SEQ ID NO:73; and a CDR3 comprising the amino acid sequence of SEQ ID NO:74. In some aspects, the VL that specifically binds to Trop2 comprises a CDR1 comprising the amino acid sequence of SEQ ID NO:83; a CDR2 comprising the amino acid sequence of SEQ ID NO:84; and a CDR3 comprising the amino acid sequence of SEQ ID NO:85; and the VH that specifically binds to Trop2 comprises a CDR1 comprising the amino acid sequence of SEQ ID NO:80; a CDR2 comprising the amino acid sequence of SEQ ID NO:81; and a CDR3 comprising the amino acid sequence of SEQ ID NO:82. In some aspects, the VL that specifically binds to Trop2 comprises a CDR1 comprising the amino acid sequence of SEQ ID NO:91; a CDR2 comprising the amino acid sequence of SEQ ID NO:92; and a CDR3 comprising the amino acid sequence of SEQ ID NO:93; and the VH that specifically binds to Trop2 comprises a CDR1 comprising the amino acid sequence of SEQ ID NO:88; a CDR2 comprising the amino acid sequence of SEQ ID NO:89; and a CDR3 comprising the amino acid sequence of SEQ ID NO:90. In some aspects, the VL that specifically binds to CD3 comprises a CDR1 comprising the amino acid sequence of SEQ ID NO: 99; a CDR2 comprising the amino acid sequence of SEQ ID NO:100; and a CDR3 comprising the amino acid sequence of SEQ ID NO:101; and the VH that specifically binds to CD3 comprises a CDR1 comprising the amino acid sequence of SEQ ID NO:96; a CDR2 comprising the amino acid sequence of SEQ ID NO:97; and a CDR3 comprising the amino acid sequence of SEQ ID NO:98. In some aspects, the VL that specifically binds to CD20 comprises a CDR1 comprising the amino acid sequence of SEQ ID NO: 107; a CDR2 comprising the amino acid sequence of SEQ ID NO:108; and a CDR3 comprising the amino acid sequence of SEQ ID NO:109; and the VH that specifically binds to CD20 comprises a CDR1 comprising the amino acid sequence of SEQ ID NO:104; a CDR2 comprising the amino acid sequence of SEQ ID NO:105; and a CDR3 comprising the amino acid sequence of SEQ ID NO:106. In some aspects, the VL that specifically binds to CD20 comprises a CDR1 comprising the amino acid sequence of SEQ ID NO: 115; a CDR2 comprising the amino acid sequence of SEQ ID NO:116; and a CDR3 comprising the amino acid sequence of SEQ ID NO:117; and the VH that specifically binds to CD20 comprises a CDR1 comprising the amino acid sequence of SEQ ID NO:112; a CDR2 comprising the amino acid sequence of SEQ ID NO:113; and a CDR3 comprising the amino acid sequence of SEQ ID NO:114. In some aspects, the VL that specifically binds to CD19 comprises a CDR1 comprising the amino acid sequence of SEQ ID NO: 123; a CDR2 comprising the amino acid sequence of SEQ ID NO:124; and a CDR3 comprising the amino acid sequence of SEQ ID NO:125; and the VH that specifically binds to CD19 comprises a CDR1 comprising the amino acid sequence of SEQ ID NO:120; a CDR2 comprising the amino acid sequence of SEQ ID NO:121; and a CDR3 comprising the amino acid sequence of SEQ ID NO:122. In some aspects, the VL that specifically binds to CD19 comprises a CDR1 comprising the amino acid sequence of SEQ ID NO: 131; a CDR2 comprising the amino acid sequence of SEQ ID NO:132; and a CDR3 comprising the amino acid sequence of SEQ ID NO:133; and the VH that specifically binds to CD19 comprises a CDR1 comprising the amino acid sequence of SEQ ID NO:128; a CDR2 comprising the amino acid sequence of SEQ ID NO:129; and a CDR3 comprising the amino acid sequence of SEQ ID NO:130. In some aspects, the VL that specifically binds to Trop2 comprises a CDR1 comprising the amino acid sequence of SEQ ID NO: 385; a CDR2 comprising the amino acid sequence of SEQ ID NO:386 (YTS); and a CDR3 comprising the amino acid sequence of SEQ ID NO:387; and the VH that specifically binds to Trop2 comprises a CDR1 comprising the amino acid sequence of SEQ ID NO:389; a CDR2 comprising the amino acid sequence of SEQ ID NO:390; and a CDR3 comprising the amino acid sequence of SEQ ID NO:391. In some aspects, the VL that specifically binds to Trop2 comprises a CDR1 comprising the amino acid sequence of SEQ ID NO: 405; a CDR2 comprising the amino acid sequence of SEQ ID NO:406 (YTS); and a CDR3 comprising the amino acid sequence of SEQ ID NO:407; and the VH that specifically binds to Trop2 comprises a CDR1 comprising the amino acid sequence of SEQ ID NO:409; a CDR2 comprising the amino acid sequence of SEQ ID NO:410; and a CDR3 comprising the amino acid sequence of SEQ ID NO:411. In some aspects, the VL that specifically binds to Trop2 comprises a CDR1 comprising the amino acid sequence of SEQ ID NO: 415; a CDR2 comprising the amino acid sequence of SEQ ID NO:416 (YTS); and a CDR3 comprising the amino acid sequence of SEQ ID NO:417; and the VH that specifically binds to Trop2 comprises a CDR1 comprising the amino acid sequence of SEQ ID NO:419; a CDR2 comprising the amino acid sequence of SEQ ID NO:420; and a CDR3 comprising the amino acid sequence of SEQ ID NO:421. In some aspects, the VL that specifically binds to Trop2 comprises a CDR1 comprising the amino acid sequence of SEQ ID NO: 441; a CDR2 comprising the amino acid sequence of SEQ ID NO:442 (YTS); and a CDR3 comprising the amino acid sequence of SEQ ID NO:443; and the VH that specifically binds to Trop2 comprises a CDR1 comprising the amino acid sequence of SEQ ID NO:429; a CDR2 comprising the amino acid sequence of SEQ ID NO:430; and a CDR3 comprising the amino acid sequence of SEQ ID NO:431. In some aspects, the VL that specifically binds to Trop2 comprises a CDR1 comprising the amino acid sequence of SEQ ID NO: 452; a CDR2 comprising the amino acid sequence of SEQ ID NO:453 (YTS); and a CDR3 comprising the amino acid sequence of SEQ ID NO:454; and the VH that specifically binds to Trop2 comprises a CDR1 comprising the amino acid sequence of SEQ ID NO:429; a CDR2 comprising the amino acid sequence of SEQ ID NO:430; and a CDR3 the amino acid sequence of SEQ ID NO: 431. In some aspects, the VL that specifically binds to Trop2 comprises a CDR1 comprising the amino acid sequence of SEQ ID NO: 462; a CDR2 comprising the amino acid sequence of SEQ ID NO:463 (YTS); and a CDR3 comprising the amino acid sequence of SEQ ID NO:464; and the VH that specifically binds to Trop2 comprises a CDR1 comprising the amino acid sequence of SEQ ID NO:429; a CDR2 comprising the amino acid sequence of SEQ ID NO:430; and a CDR3 comprising the amino acid sequence of SEQ ID NO:431. In some aspects, the VL that specifically binds to Trop2 comprises a CDR1 comprising the amino acid sequence of SEQ ID NO: 472; a CDR2 comprising the amino acid sequence of SEQ ID NO:473 (YTS); and a CDR3 comprising the amino acid sequence of SEQ ID NO:474; and the VH that specifically binds to Trop2 comprises a CDR1 comprising the amino acid sequence of SEQ ID NO:429; a CDR2 comprising the amino acid sequence of SEQ ID NO:430; and a CDR3 comprising the amino acid sequence of SEQ ID NO:431. In some aspects, the VL that specifically binds to Trop2 comprises a CDR1 comprising the amino acid sequence of SEQ ID NO: 655; a CDR2 comprising the amino acid sequence of SEQ ID NO:656; and a CDR3 the amino acid sequence of SEQ ID NO:657; and the VH that specifically binds to Trop2 comprises a CDR1 comprising the amino acid sequence of SEQ ID NO:659; a CDR2 comprising the amino acid sequence of SEQ ID NO:660; and a CDR3 comprising the amino acid sequence of SEQ ID NO:661. In some aspects, the VL that specifically binds to Trop2 comprises a CDR1 comprising the amino acid sequence of SEQ ID NO: 691; a CDR2 comprising the amino acid sequence of SEQ ID NO:692; and a CDR3 comprising the amino acid sequence of SEQ ID NO:693; and the VH that specifically binds to Trop2 comprises a CDR1 comprising the amino acid sequence of SEQ ID NO:695; a CDR2 comprising the amino acid sequence of SEQ ID NO:696; and a CDR3 comprising the amino acid sequence of SEQ ID NO:697. In some aspects, the VL that specifically binds to Trop2 comprises a CDR1 comprising the amino acid sequence of SEQ ID NO: 727; a CDR2 comprising the amino acid sequence of SEQ ID NO:728; and a CDR3 comprising the amino acid sequence of SEQ ID NO:729; and the VH that specifically binds to Trop2 comprises a CDR1 comprising the amino acid sequence of SEQ ID NO:731; a CDR2 comprising the amino acid sequence of SEQ ID NO:732; and a CDR3 comprising the amino acid sequence of SEQ ID NO:733. In some aspects, the VL that specifically binds to HER2 comprises a CDR1 comprising the amino acid sequence of SEQ ID NO: 393; a CDR2 comprising the amino acid sequence of SEQ ID NO:394; and a CDR3 comprising the amino acid sequence of SEQ ID NO:395; and the VH that specifically binds to HER2 comprises a CDR1 comprising the amino acid sequence of SEQ ID NO:397; a CDR2 comprising the amino acid sequence of SEQ ID NO:398; and a CDR3 comprising the amino acid sequence of SEQ ID NO:399. In some aspects, the VL that specifically binds to CD20 comprises a CDR1 comprising the amino acid sequence of SEQ ID NO: 486; a CDR2 comprising the amino acid sequence of SEQ ID NO:487 (AT); and a CDR3 comprising the amino acid sequence of SEQ ID NO:488; and the VH that specifically binds to CD20 comprises a CDR1 comprising the amino acid sequence of SEQ ID NO:508; a CDR2 comprising the amino acid sequence of SEQ ID NO:509; and a CDR3 comprising the amino acid sequence of SEQ ID NO:510. In some aspects, the VL that specifically binds to CD20 comprises a CDR1 comprising the amino acid sequence of SEQ ID NO: 504; a CDR2 comprising the amino acid sequence of SEQ ID NO:505 (AT); and a CDR3 comprising the amino acid sequence of SEQ ID NO:506; and the VH that specifically binds to CD20 comprises a CDR1 comprising the amino acid sequence of SEQ ID NO:508; a CDR2 comprising the amino acid sequence of SEQ ID NO:509; and a CDR3 comprising the amino acid sequence of SEQ ID NO:510. In some aspects, the VL that specifically binds to CD20 comprises a CDR1 comprising the amino acid sequence of SEQ ID NO: 536; a CDR2 comprising the amino acid sequence of SEQ ID NO:537; and a CDR3 comprising the amino acid sequence of SEQ ID NO:538; and the VH that specifically binds to CD20 comprises a CDR1 comprising the amino acid sequence of SEQ ID NO:540; a CDR2 comprising the amino acid sequence of SEQ ID NO:541; and a CDR3 comprising the amino acid sequence of SEQ ID NO:542. In some aspects, the VL that specifically binds to CD20 comprises a CDR1 comprising the amino acid sequence of SEQ ID NO: 548; a CDR2 comprising the amino acid sequence of SEQ ID NO:549; and a CDR3 comprising the amino acid sequence of SEQ ID NO:550; and the VH that specifically binds to CD20 comprises a CDR1 comprising the amino acid sequence of SEQ ID NO:552; a CDR2 comprising the amino acid sequence of SEQ ID NO:553; and a CDR3 comprising the amino acid sequence of SEQ ID NO:554. In some aspects, the VL that specifically binds to CD20 comprises a CDR1 comprising the amino acid sequence of SEQ ID NO: 753; a CDR2 comprising the amino acid sequence of SEQ ID NO:754; and a CDR3 comprising the amino acid sequence of SEQ ID NO:755; and the VH that specifically binds to CD20 comprises a CDR1 comprising the amino acid sequence of SEQ ID NO:757; a CDR2 comprising the amino acid sequence of SEQ ID NO:758; and a CDR3 comprising the amino acid sequence of SEQ ID NO:759. In some aspects, the VL that specifically binds to CD20 comprises a CDR1 comprising the amino acid sequence of SEQ ID NO: 789; a CDR2 comprising the amino acid sequence of SEQ ID NO:790; and a CDR3 comprising the amino acid sequence of SEQ ID NO:791; and the VH that specifically binds to CD20 comprises a CDR1 comprising the amino acid sequence of SEQ ID NO:793; a CDR2 comprising the amino acid sequence of SEQ ID NO:794; and a CDR3 comprising the amino acid sequence of SEQ ID NO:795. In some aspects, the VL that specifically binds to CD20 comprises a CDR1 comprising the amino acid sequence of SEQ ID NO: 825; a CDR2 comprising the amino acid sequence of SEQ ID NO:826; and a CDR3 comprising the amino acid sequence of SEQ ID NO:827; and the VH that specifically binds to CD20 comprises a CDR1 comprising the amino acid sequence of SEQ ID NO:829; a CDR2 comprising the amino acid sequence of SEQ ID NO:830; and a CDR3 comprising the amino acid sequence of SEQ ID NO:831. In some aspects, the VL that specifically binds to CD20 comprises a CDR1 comprising the amino acid sequence of SEQ ID NO: 871; a CDR2 comprising the amino acid sequence of SEQ ID NO:872; and a CDR3 comprising the amino acid sequence of SEQ ID NO:873. In some aspects, the VL that specifically binds to CD20 comprises a CDR1 comprising the amino acid sequence of SEQ ID NO: 879; a CDR2 comprising the amino acid sequence of SEQ ID NO:880; and a CDR3 comprising the amino acid sequence of SEQ ID NO:881; and the VH that specifically binds to CD20 comprises a CDR1 comprising the amino acid sequence of SEQ ID NO:875; a CDR2 comprising the amino acid sequence of SEQ ID NO:876; and a CDR3 comprising the amino acid sequence of SEQ ID NO:877. In some aspects, the VL that specifically binds to CD19 comprises a CDR1 comprising the amino acid sequence of SEQ ID NO: 490; a CDR2 comprising the amino acid sequence of SEQ ID NO:491 (AT); and a CDR3 comprising the amino acid sequence of SEQ ID NO:492; and the VH that specifically binds to CD19 comprises a CDR1 comprising the amino acid sequence of SEQ ID NO:500; a CDR2 comprising the amino acid sequence of SEQ ID NO:501; and a CDR3 comprising the amino acid sequence of SEQ ID NO:502. In some aspects, the VL that specifically binds to CD19 comprises a CDR1 comprising the amino acid sequence of SEQ ID NO: 496; a CDR2 comprising the amino acid sequence of SEQ ID NO:497 (AT); and a CDR3 comprising the amino acid sequence of SEQ ID NO:498; and the VH that specifically binds to CD19 comprises a CDR1 comprising the amino acid sequence of SEQ ID NO:500; a CDR2 comprising the amino acid sequence of SEQ ID NO:501; and a CDR3 comprising the amino acid sequence of SEQ ID NO:502. In some aspects, the VL that specifically binds to CD19 comprises a CDR1 comprising the amino acid sequence of SEQ ID NO: 745; a CDR2 comprising the amino acid sequence of SEQ ID NO: 746; and a CDR3 comprising the amino acid sequence of SEQ ID NO:747; and the VH that specifically binds to CD19 comprises a CDR1 comprising the amino acid sequence of SEQ ID NO:749; a CDR2 comprising the amino acid sequence of SEQ ID NO:750; and a CDR3 comprising the amino acid sequence of SEQ ID NO:751. In some aspects, the VL that specifically binds to CD19 comprises a CDR1 comprising the amino acid sequence of SEQ ID NO: 781; a CDR2 comprising the amino acid sequence of SEQ ID NO: 782; and a CDR3 comprising the amino acid sequence of SEQ ID NO:783; and the VH that specifically binds to CD19 comprises a CDR1 comprising the amino acid sequence of SEQ ID NO:785; a CDR2 comprising the amino acid sequence of SEQ ID NO:786; and a CDR3 the amino acid sequence of SEQ ID NO:787. In some aspects, the VL that specifically binds to CD19 comprises a CDR1 comprising the amino acid sequence of SEQ ID NO: 817; a CDR2 comprising the amino acid sequence of SEQ ID NO:818; and a CDR3 comprising the amino acid sequence of SEQ ID NO:819; and the VH that specifically binds to CD19 comprises a CDR1 comprising the amino acid sequence of SEQ ID NO:821; a CDR2 comprising the amino acid sequence of SEQ ID NO:822; and a CDR3 comprising the amino acid sequence of SEQ ID NO:823. In some aspects, the VL that specifically binds to CD28 comprises a CDR1 comprising the amino acid sequence of SEQ ID NO: 645; a CDR2 comprising the amino acid sequence of SEQ ID NO:646; and a CDR3 comprising the amino acid sequence of SEQ ID NO:647; and the VH that specifically binds to CD28 comprises a CDR1 comprising the amino acid sequence of SEQ ID NO:649; a CDR2 comprising the amino acid sequence of SEQ ID NO: 650; and a CDR3 comprising the amino acid sequence of SEQ ID NO:651. In some aspects, the VL that specifically binds to CD28 comprises a CDR1 comprising the amino acid sequence of SEQ ID NO: 681; a CDR2 comprising the amino acid sequence of SEQ ID NO:682; and a CDR3 comprising the amino acid sequence of SEQ ID NO:683; and the VH that specifically binds to CD28 comprises a CDR1 comprising the amino acid sequence of SEQ ID NO:685; a CDR2 comprising the amino acid sequence of SEQ ID NO:686; and a CDR3 comprising the amino acid sequence of SEQ ID NO:687. In some aspects, the VL that specifically binds to CD28 comprises a CDR1 comprising the amino acid sequence of SEQ ID NO: 771; a CDR2 comprising the amino acid sequence of SEQ ID NO:772; and a CDR3 comprising the amino acid sequence of SEQ ID NO:773; and the VH that specifically binds to CD28 comprises a CDR1 comprising the amino acid sequence of SEQ ID NO:775; a CDR2 comprising the amino acid sequence of SEQ ID NO:776; and a CDR3 comprising the amino acid sequence of SEQ ID NO:777. In some aspects, the VL that specifically binds to CD28 comprises a CDR1 comprising the amino acid sequence of SEQ ID NO: 843; a CDR2 comprising the amino acid sequence of SEQ ID NO:844; and a CDR3 comprising the amino acid sequence of SEQ ID NO:845; and the VH that specifically binds to CD28 comprises a CDR1 comprising the amino acid sequence of SEQ ID NO:847; a CDR2 comprising the amino acid sequence of SEQ ID NO:848; and a CDR3 comprising the amino acid sequence of SEQ ID NO:849. In some aspects, the VL that specifically binds to BCMA comprises a CDR1 comprising the amino acid sequence of SEQ ID NO: 621; a CDR2 comprising the amino acid sequence of SEQ ID NO:622; and a CDR3 comprising the amino acid sequence of SEQ ID NO:623; and the VH that specifically binds to BCMA comprises a CDR1 comprising the amino acid sequence of SEQ ID NO:625; a CDR2 comprising the amino acid sequence of SEQ ID NO:626; and a CDR3 comprising the amino acid sequence of SEQ ID NO:627. In some aspects, the VL that specifically binds to cMet comprises a CDR1 comprising the amino acid sequence of SEQ ID NO: 637; a CDR2 comprising the amino acid sequence of SEQ ID NO:638; and a CDR3 comprising the amino acid sequence of SEQ ID NO:639; and the VH that specifically binds to cMet comprises a CDR1 comprising the amino acid sequence of SEQ ID NO:641; a CDR2 comprising the amino acid sequence of SEQ ID NO:642; and a CDR3 comprising the amino acid sequence of SEQ ID NO:643. In some aspects, the VL that specifically binds to cMet comprises a CDR1 comprising the amino acid sequence of SEQ ID NO: 673; a CDR2 comprising the amino acid sequence of SEQ ID NO:674; and a CDR3 comprising the amino acid sequence of SEQ ID NO:675; and the VH that specifically binds to cMet comprises a CDR1 comprising the amino acid sequence of SEQ ID NO:677; a CDR2 comprising the amino acid sequence of SEQ ID NO:678; and a CDR3 comprising the amino acid sequence of SEQ ID NO:679. In some aspects, the VL that specifically binds to cMet comprises a CDR1 comprising the amino acid sequence of SEQ ID NO: 709; a CDR2 comprising the amino acid sequence of SEQ ID NO:710; and a CDR3 comprising the amino acid sequence of SEQ ID NO:711; and the VH that specifically binds to cMet comprises a CDR1 comprising the amino acid sequence of SEQ ID NO:713; a CDR2 comprising the amino acid sequence of SEQ ID NO:714; and a CDR3 comprising the amino acid sequence of SEQ ID NO:715.

In some aspects, the VL that specifically binds to the TAA comprises an amino acid sequence having at least 80%, at least 85%, at least 90%, at least 95%, or 100% identity to any one of SEQ ID NOs:71, 79, 87, 95, 103, 111, 119, 127, 135, 384, 404, 414, 440, 451, 461, 471, 583, 654, 690, 726, 392, 402, 485, 503, 535, 547, 752, 788, 824, 882, 870, 878, 489, 495, 543, 555, 744, 780, 816, 527, 644, 680, 770, 842, 620, 636, 672 and 708; and the VH that specifically binds to the TAA comprises an amino acid sequence having at least 80%, at least 85%, at least 90%, at least 95%, or 100% identity to any one of SEQ ID NOs:70, 78, 86, 94, 102, 110, 118, 126, 134, 388, 408, 418, 428, 584, 658, 694, 730, 396, 403, 507, 539, 551, 756, 792, 828, 883, 866, 874, 499, 544, 556, 748, 784, 820, 528, 648, 684, 774, 846, 624, 640, 676 and 712. In some aspects, the VL that specifically binds to CD38 comprises an amino acid sequence having at least 80% identity (such as at least 85%, 90%, 91%, 92%, 93%, 94%, 95%, 96%, 97%, 98%, 99% or 100% identity) to SEQ ID NO:71; and the VH that specifically binds to CD38 comprises an amino acid sequence having at least 80% identity (such as at least 85%, 90%, 91%, 92%, 93%, 94%, 95%, 96%, 97%, 98%, 99% or 100% identity) to SEQ ID NO:70. In some aspects, the VL that specifically binds to cMet comprises an amino acid sequence having at least 80% identity (such as at least 85%, 90%, 91%, 92%, 93%, 94%, 95%, 96%, 97%, 98%, 99% or 100% identity) to SEQ ID NO:79; and the VH that specifically binds to cMet comprises an amino acid sequence having at least 80% identity (such as at least 85%, 90%, 91%, 92%, 93%, 94%, 95%, 96%, 97%, 98%, 99% or 100% identity) to SEQ ID NO:78. In some aspects, the VL that specifically binds to Trop2 comprises an amino acid sequence having at least 80% identity (such as at least 85%, 90%, 91%, 92%, 93%, 94%, 95%, 96%, 97%, 98%, 99% or 100% identity) to SEQ ID NO:87; and the VH that specifically binds to Trop2 comprises an amino acid sequence having at least 80% identity (such as at least 85%, 90%, 91%, 92%, 93%, 94%, 95%, 96%, 97%, 98%, 99% or 100% identity) to SEQ ID NO:86. In some aspects, the VL that specifically binds to Trop2 comprises an amino acid sequence having at least 80% identity (such as at least 85%, 90%, 91%, 92%, 93%, 94%, 95%, 96%, 97%, 98%, 99% or 100% identity) to SEQ ID NO:95; and the VH that specifically binds to Trop2 comprises an amino acid sequence having at least 80% identity (such as at least 85%, 90%, 91%, 92%, 93%, 94%, 95%, 96%, 97%. 98%, 99% or 100% identity) to SEQ ID NO:94. In some aspects, the VL that specifically binds to CD3 comprises an amino acid sequence having at least 80% identity (such as at least 85%, 90%, 91%, 92%, 93%, 94%, 95%, 96%, 97%, 98%, 99% or 100% identity) to SEQ ID NO:103; and the VH that specifically binds to CD3 comprises an amino acid sequence having at least 80% identity (such as at least 85%, 90%, 91%, 92%, 93%, 94%, 95%, 96%, 97%, 98%, 99% or 100% identity) to SEQ ID NO:102. In some aspects, the VL that specifically binds to CD20 comprises an amino acid sequence having at least 80% identity (such as at least 85%, 90%, 91%, 92%, 93%, 94%, 95%, 96%, 97%, 98%, 99% or 100% identity) to SEQ ID NO:111; and the VH that specifically binds to CD20 comprises an amino acid sequence having at least 80% identity (such as at least 85%, 90%, 91%, 92%, 93%, 94%, 95%, 96%, 97%, 98%, 99% or 100% identity) to SEQ ID NO:110. In some aspects, the VL that specifically binds to CD20 comprises an amino acid sequence having at least 80% identity (such as at least 85%, 90%, 91%, 92%, 93%, 94%, 95%, 96%, 97%, 98%, 99% or 100% identity) to SEQ ID NO:119; and the VH that specifically binds to CD20 comprises an amino acid sequence having at least 80% identity (such as at least 85%, 90%, 91%, 92%, 93%, 94%, 95%, 96%, 97%, 98%, 99% or 100% identity) to SEQ ID NO:118. In some aspects, the VL that specifically binds to CD20 comprises an amino acid sequence having at least 80% identity (such as at least 85%, 90%, 91%, 92%, 93%, 94%, 95%, 96%, 97%, 98%, 99% or 100% identity) to SEQ ID NO:111; and the VH that specifically binds to CD20 comprises an amino acid sequence having at least 80% identity (such as at least 85%, 90%, 91%, 92%, 93%, 94%, 95%, 96%, 97%, 98%, 99% or 100% identity) to SEQ ID NO:118. In some aspects, the VL that specifically binds to CD20 comprises an amino acid sequence having at least 80% identity (such as at least 85%, 90%, 91%, 92%, 93%, 94%, 95%, 96%, 97%, 98%, 99% or 100% identity) to SEQ ID NO:119; and the VH that specifically binds to CD20 comprises an amino acid sequence having at least 80% identity (such as at least 85%, 90%, 91%, 92%, 93%, 94%, 95%, 96%, 97%, 98%, 99% or 100% identity) to SEQ ID NO:110. In some aspects, the VL that specifically binds to CD19 comprises an amino acid sequence having at least 80% identity (such as at least 85%, 90%, 91%, 92%, 93%, 94%, 95%, 96%, 97%, 98%, 99% or 100% identity) to SEQ ID NO:127; and the VH that specifically binds to CD19 comprises an amino acid sequence having at least 80% identity (such as at least 85%, 90%, 91%, 92%, 93%, 94%, 95%, 96%, 97%, 98%, 99% or 100% identity) to SEQ ID NO:126. In some aspects, the VL that specifically binds to CD19 comprises an amino acid sequence having at least 80% identity (such as at least 85%, 90%, 91%, 92%, 93%, 94%, 95%, 96%, 97%, 98%, 99% or 100% identity) to SEQ ID NO:135 or 882; and the VH that specifically binds to CD19 comprises an amino acid sequence having at least 80% identity (such as at least 85%, 90%, 91%, 92%, 93%, 94%, 95%, 96%, 97%, 98%, 99% or 100% identity) to SEQ ID NO:134 or 883. In some aspects, the VL that specifically binds to Trop2 comprises an amino acid sequence having at least 80% identity (such as at least 85%, 90%, 91%, 92%, 93%, 94%, 95%, 96%, 97%, 98%, 99% or 100% identity) to SEQ ID NO: 384; and the VH that specifically binds to Trop2 comprises an amino acid sequence having at least 80% identity (such as at least 85%, 90%, 91%, 92%, 93%, 94%, 95%, 96%, 97%, 98%, 99% or 100% identity) to SEQ ID NO: 388. In some aspects, the VL that specifically binds to Trop2 comprises an amino acid sequence having at least 80% identity (such as at least 85%, 90%, 91%, 92%, 93%, 94%, 95%, 96%, 97%, 98%, 99% or 100% identity) to SEQ ID NO: 404; and the VH that specifically binds to Trop2 comprises an amino acid sequence having at least 80% identity (such as at least 85%, 90%, 91%, 92%, 93%, 94%, 95%, 96%, 97%, 98%, 99% or 100% identity) to SEQ ID NO: 408. In some aspects, the VL that specifically binds to Trop2 comprises an amino acid sequence having at least 80% identity (such as at least 85%, 90%, 91%, 92%, 93%, 94%, 95%, 96%, 97%, 98%, 99% or 100% identity) to SEQ ID NO: 414; and the VH that specifically binds to Trop2 comprises an amino acid sequence having at least 80% identity (such as at least 85%, 90%, 91%, 92%, 93%, 94%, 95%, 96%, 97%, 98%, 99% or 100% identity) to SEQ ID NO: 418. In some aspects, the VL that specifically binds to Trop2 comprises an amino acid sequence having at least 80% identity (such as at least 85%, 90%, 91%, 92%, 93%, 94%, 95%, 96%, 97%, 98%, 99% or 100% identity) to SEQ ID NO: 440; and the VH that specifically binds to Trop2 comprises an amino acid sequence having at least 80% identity (such as at least 85%, 90%, 91%, 92%, 93%, 94%, 95%, 96%, 97%, 98%, 99% or 100% identity) to SEQ ID NO: 428. In some aspects, the VL that specifically binds to Trop2 comprises an amino acid sequence having at least 80% identity (such as at least 85%, 90%, 91%, 92%, 93%, 94%, 95%, 96%, 97%, 98%, 99% or 100% identity) to SEQ ID NO: 451; and the VH that specifically binds to Trop2 comprises an amino acid sequence having at least 80% identity (such as at least 85%, 90%, 91%, 92%, 93%, 94%, 95%, 96%, 97%, 98%, 99% or 100% identity) to SEQ ID NO: 428. In some aspects, the VL that specifically binds to Trop2 comprises an amino acid sequence having at least 80% identity (such as at least 85%, 90%, 91%, 92%, 93%, 94%, 95%, 96%, 97%, 98%, 99% or 100% identity) to SEQ ID NO: 461; and the VH that specifically binds to Trop2 comprises an amino acid sequence having at least 80% identity (such as at least 85%, 90%, 91%, 92%, 93%, 94%, 95%, 96%, 97%, 98%, 99% or 100% identity) to SEQ ID NO: 428. In some aspects, the VL that specifically binds to Trop2 comprises an amino acid sequence having at least 80% identity (such as at least 85%, 90%, 91%, 92%, 93%, 94%, 95%, 96%, 97%, 98%, 99% or 100% identity) to SEQ ID NO: 471; and the VH that specifically binds to Trop2 comprises an amino acid sequence having at least 80% identity (such as at least 85%, 90%, 91%, 92%, 93%, 94%, 95%, 96%, 97%, 98%, 99% or 100% identity) to SEQ ID NO: 428. In some aspects, the VL that specifically binds to Trop2 comprises an amino acid sequence having at least 80% identity (such as at least 85%, 90%, 91%, 92%, 93%, 94%, 95%, 96%, 97%, 98%, 99% or 100% identity) to SEQ ID NO: 583; and the VH that specifically binds to Trop2 comprises an amino acid sequence having at least 80% identity (such as at least 85%, 90%, 91%, 92%, 93%, 94%, 95%, 96%, 97%, 98%, 99% or 100% identity) to SEQ ID NO: 584. In some aspects, the VL that specifically binds to Trop2 comprises an amino acid sequence having at least 80% identity (such as at least 85%, 90%, 91%, 92%, 93%, 94%, 95%, 96%, 97%, 98%, 99% or 100% identity) to SEQ ID NO: 654; and the VH that specifically binds to Trop2 comprises an amino acid sequence having at least 80% identity (such as at least 85%, 90%, 91%, 92%, 93%, 94%, 95%, 96%, 97%, 98%, 99% or 100% identity) to SEQ ID NO: 658. In some aspects, the VL that specifically binds to Trop2 comprises an amino acid sequence having at least 80% identity (such as at least 85%, 90%, 91%, 92%, 93%, 94%, 95%, 96%, 97%, 98%, 99% or 100% identity) to SEQ ID NO: 690; and the VH that specifically binds to Trop2 comprises an amino acid sequence having at least 80% identity (such as at least 85%, 90%, 91%, 92%, 93%, 94%, 95%, 96%, 97%, 98%, 99% or 100% identity) to SEQ ID NO: 694. In some aspects, the VL that specifically binds to Trop2 comprises an amino acid sequence having at least 80% identity (such as at least 85%, 90%, 91%, 92%, 93%, 94%, 95%, 96%, 97%, 98%, 99% or 100% identity) to SEQ ID NO: 726; and the VH that specifically binds to Trop2 comprises an amino acid sequence having at least 80% identity (such as at least 85%, 90%, 91%, 92%, 93%, 94%, 95%, 96%, 97%, 98%, 99% or 100% identity) to SEQ ID NO: 730. In some aspects, the VL that specifically binds to CD20 comprises an amino acid sequence having at least 80% identity (such as at least 85%, 90%, 91%, 92%, 93%, 94%, 95%, 96%, 97%, 98%, 99% or 100% identity) to SEQ ID NO: 485; and the VH that specifically binds to CD20 comprises an amino acid sequence having at least 80% identity (such as at least 85%, 90%, 91%, 92%, 93%, 94%, 95%, 96%, 97%, 98%, 99% or 100% identity) to SEQ ID NO: 507. In some aspects, the VL that specifically binds to CD19 comprises an amino acid sequence having at least 80% identity (such as at least 85%, 90%, 91%, 92%, 93%, 94%, 95%, 96%, 97%, 98%, 99% or 100% identity) to SEQ ID NO: 489; and the VH that specifically binds to CD19 comprises an amino acid sequence having at least 80% identity (such as at least 85%, 90%, 91%, 92%, 93%, 94%, 95%, 96%, 97%, 98%, 99% or 100% identity) to SEQ ID NO: 499. In some aspects, the VL that specifically binds to HER2 comprises an amino acid sequence having at least 80% identity (such as at least 85%, 90%, 91%, 92%, 93%, 94%, 95%, 96%, 97%, 98%, 99% or 100% identity) to SEQ ID NO: 392; and the VH that specifically binds to HER2 comprises an amino acid sequence having at least 80% identity (such as at least 85%, 90%, 91%, 92%, 93%, 94%, 95%, 96%, 97%, 98%, 99% or 100% identity) to SEQ ID NO: 396. In some aspects, the VL that specifically binds to HER2 comprises an amino acid sequence having at least 80% identity (such as at least 85%, 90%, 91%, 92%, 93%, 94%, 95%, 96%, 97%, 98%, 99% or 100% identity) to SEQ ID NO: 402; and the VH that specifically binds to HER2 comprises an amino acid sequence having at least 80% identity (such as at least 85%, 90%, 91%, 92%, 93%, 94%, 95%, 96%, 97%, 98%, 99% or 100% identity) to SEQ ID NO: 403. In some aspects, the VL that specifically binds to CD20 comprises an amino acid sequence having at least 80% identity (such as at least 85%, 90%, 91%, 92%, 93%, 94%, 95%, 96%, 97%, 98%, 99% or 100% identity) to SEQ ID NO: 503; and the VH that specifically binds to CD20 comprises an amino acid sequence having at least 80% identity (such as at least 85%, 90%, 91%, 92%, 93%, 94%, 95%, 96%, 97%, 98%, 99% or 100% identity) to SEQ ID NO: 507. In some aspects, the VL that specifically binds to CD20 comprises an amino acid sequence having at least 80% identity (such as at least 85%, 90%, 91%, 92%, 93%, 94%, 95%, 96%, 97%, 98%, 99% or 100% identity) to SEQ ID NO: 535; and the VH that specifically binds to CD20 comprises an amino acid sequence having at least 80% identity (such as at least 85%, 90%, 91%, 92%, 93%, 94%, 95%, 96%, 97%, 98%, 99% or 100% identity) to SEQ ID NO: 539. In some aspects, the VL that specifically binds to CD20 comprises an amino acid sequence having at least 80% identity (such as at least 85%, 90%, 91%, 92%, 93%, 94%, 95%, 96%, 97%, 98%, 99% or 100% identity) to SEQ ID NO: 547; and the VH that specifically binds to CD20 comprises an amino acid sequence having at least 80% identity (such as at least 85%, 90%, 91%, 92%, 93%, 94%, 95%, 96%, 97%, 98%, 99% or 100% identity) to SEQ ID NO: 551. In some aspects, the VL that specifically binds to CD20 comprises an amino acid sequence having at least 80% identity (such as at least 85%, 90%, 91%, 92%, 93%, 94%, 95%, 96%, 97%, 98%, 99% or 100% identity) to SEQ ID NO: 752; and the VH that specifically binds to CD20 comprises an amino acid sequence having at least 80% identity (such as at least 85%, 90%, 91%, 92%, 93%, 94%, 95%, 96%, 97%, 98%, 99% or 100% identity) to SEQ ID NO: 756. In some aspects, the VL that specifically binds to CD20 comprises an amino acid sequence having at least 80% identity (such as at least 85%, 90%, 91%, 92%, 93%, 94%, 95%, 96%, 97%, 98%, 99% or 100% identity) to SEQ ID NO: 788; and the VH that specifically binds to CD20 comprises an amino acid sequence having at least 80% identity (such as at least 85%, 90%, 91%, 92%, 93%, 94%, 95%, 96%, 97%, 98%, 99% or 100% identity) to SEQ ID NO: 792. In some aspects, the VL that specifically binds to CD20 comprises an amino acid sequence having at least 80% identity (such as at least 85%, 90%, 91%, 92%, 93%, 94%, 95%, 96%, 97%, 98%, 99% or 100% identity) to SEQ ID NO: 824; and the VH that specifically binds to CD20 comprises an amino acid sequence having at least 80% identity (such as at least 85%, 90%, 91%, 92%, 93%, 94%, 95%, 96%, 97%, 98%, 99% or 100% identity) to SEQ ID NO: 828. In some aspects, the VL that specifically binds to CD20 comprises an amino acid sequence having at least 80% identity (such as at least 85%, 90%, 91%, 92%, 93%, 94%, 95%, 96%, 97%, 98%, 99% or 100% identity) to SEQ ID NO: 878; and the VH that specifically binds to CD20 comprises an amino acid sequence having at least 80% identity (such as at least 85%, 90%, 91%, 92%, 93%, 94%, 95%, 96%, 97%, 98%, 99% or 100% identity) to SEQ ID NO: 874. In some aspects, the VL that specifically binds to CD19 comprises an amino acid sequence having at least 80% identity (such as at least 85%, 90%, 91%, 92%, 93%, 94%, 95%, 96%, 97%, 98%, 99% or 100% identity) to SEQ ID NO: 495; and the VH that specifically binds to CD19 comprises an amino acid sequence having at least 80% identity (such as at least 85%, 90%, 91%, 92%, 93%, 94%, 95%, 96%, 97%, 98%, 99% or 100% identity) to SEQ ID NO: 499. In some aspects, the VL that specifically binds to CD19 comprises an amino acid sequence having at least 80% identity (such as at least 85%, 90%, 91%, 92%, 93%, 94%, 95%, 96%, 97%, 98%, 99% or 100% identity) to SEQ ID NO: 543; and the VH that specifically binds to CD19 comprises an amino acid sequence having at least 80% identity (such as at least 85%, 90%, 91%, 92%, 93%, 94%, 95%, 96%, 97%, 98%, 99% or 100% identity) to SEQ ID NO: 544. In some aspects, the VL that specifically binds to CD19 comprises an amino acid sequence having at least 80% identity (such as at least 85%, 90%, 91%, 92%, 93%, 94%, 95%, 96%, 97%, 98%, 99% or 100% identity) to SEQ ID NO: 555; and the VH that specifically binds to CD19 comprises an amino acid sequence having at least 80% identity (such as at least 85%, 90%, 91%, 92%, 93%, 94%, 95%, 96%, 97%, 98%, 99% or 100% identity) to SEQ ID NO: 556. In some aspects, the VL that specifically binds to CD19 comprises an amino acid sequence having at least 80% identity (such as at least 85%, 90%, 91%, 92%, 93%, 94%, 95%, 96%, 97%, 98%, 99% or 100% identity) to SEQ ID NO: 744; and the VH that specifically binds to CD19 comprises an amino acid sequence having at least 80% identity (such as at least 85%, 90%, 91%, 92%, 93%, 94%, 95%, 96%, 97%, 98%, 99% or 100% identity) to SEQ ID NO: 748. In some aspects, the VL that specifically binds to CD19 comprises an amino acid sequence having at least 80% identity (such as at least 85%, 90%, 91%, 92%, 93%, 94%, 95%, 96%, 97%, 98%, 99% or 100% identity) to SEQ ID NO: 780; and the VH that specifically binds to CD19 comprises an amino acid sequence having at least 80% identity (such as at least 85%, 90%, 91%, 92%, 93%, 94%, 95%, 96%, 97%, 98%, 99% or 100% identity) to SEQ ID NO: 784. In some aspects, the VL that specifically binds to CD19 comprises an amino acid sequence having at least 80% identity (such as at least 85%, 90%, 91%, 92%, 93%, 94%, 95%, 96%, 97%, 98%, 99% or 100% identity) to SEQ ID NO: 816; and the VH that specifically binds to CD19 comprises an amino acid sequence having at least 80% identity (such as at least 85%, 90%, 91%, 92%, 93%, 94%, 95%, 96%, 97%, 98%, 99% or 100% identity) to SEQ ID NO: 820. In some aspects, the VL that specifically binds to CD28 comprises an amino acid sequence having at least 80% identity (such as at least 85%, 90%, 91%, 92%, 93%, 94%, 95%, 96%, 97%, 98%, 99% or 100% identity) to SEQ ID NO: 527; and the VH that specifically binds to CD28 comprises an amino acid sequence having at least 80% identity (such as at least 85%, 90%, 91%, 92%, 93%, 94%, 95%, 96%, 97%, 98%, 99% or 100% identity) to SEQ ID NO: 528. In some aspects, the VL that specifically binds to CD28 comprises an amino acid sequence having at least 80% identity (such as at least 85%, 90%, 91%, 92%, 93%, 94%, 95%, 96%, 97%, 98%, 99% or 100% identity) to SEQ ID NO: 644; and the VH that specifically binds to CD28 comprises an amino acid sequence having at least 80% identity (such as at least 85%, 90%, 91%, 92%, 93%, 94%, 95%, 96%, 97%, 98%, 99% or 100% identity) to SEQ ID NO: 648. In some aspects, the VL that specifically binds to CD28 comprises an amino acid sequence having at least 80% identity (such as at least 85%, 90%, 91%, 92%, 93%, 94%, 95%, 96%, 97%, 98%, 99% or 100% identity) to SEQ ID NO: 680; and the VH that specifically binds to CD28 comprises an amino acid sequence having at least 80% identity (such as at least 85%, 90%, 91%, 92%, 93%, 94%, 95%, 96%, 97%, 98%, 99% or 100% identity) to SEQ ID NO: 684. In some aspects, the VL that specifically binds to CD28 comprises an amino acid sequence having at least 80% identity (such as at least 85%, 90%, 91%, 92%, 93%, 94%, 95%, 96%, 97%, 98%, 99% or 100% identity) to SEQ ID NO: 770; and the VH that specifically binds to CD28 comprises an amino acid sequence having at least 80% identity (such as at least 85%, 90%, 91%, 92%, 93%, 94%, 95%, 96%, 97%, 98%, 99% or 100% identity) to SEQ ID NO: 774. In some aspects, the VL that specifically binds to CD28 comprises an amino acid sequence having at least 80% identity (such as at least 85%, 90%, 91%, 92%, 93%, 94%, 95%, 96%, 97%, 98%, 99% or 100% identity) to SEQ ID NO: 842; and the VH that specifically binds to CD28 comprises an amino acid sequence having at least 80% identity (such as at least 85%, 90%, 91%, 92%, 93%, 94%, 95%, 96%, 97%, 98%, 99% or 100% identity) to SEQ ID NO: 846. In some aspects, the VL that specifically binds to BCMA comprises an amino acid sequence having at least 80% identity (such as at least 85%, 90%, 91%, 92%, 93%, 94%, 95%, 96%, 97%, 98%, 99% or 100% identity) to SEQ ID NO: 620; and the VH that specifically binds to BCMA comprises an amino acid sequence having at least 80% identity (such as at least 85%, 90%, 91%, 92%, 93%, 94%, 95%, 96%, 97%, 98%, 99% or 100% identity) to SEQ ID NO: 624. In some aspects, the VL that specifically binds to cMet comprises an amino acid sequence having at least 80% identity (such as at least 85%, 90%, 91%, 92%, 93%, 94%, 95%, 96%, 97%, 98%, 99% or 100% identity) to SEQ ID NO: 636; and the VH that specifically binds to cMet comprises an amino acid sequence having at least 80% identity (such as at least 85%, 90%, 91%, 92%, 93%, 94%, 95%, 96%, 97%, 98%, 99% or 100% identity) to SEQ ID NO: 640. In some aspects, the VL that specifically binds to cMet comprises an amino acid sequence having at least 80% identity (such as at least 85%, 90%, 91%, 92%, 93%, 94%, 95%, 96%, 97%, 98%, 99% or 100% identity) to SEQ ID NO: 672; and the VH that specifically binds to cMet comprises an amino acid sequence having at least 80% identity (such as at least 85%, 90%, 91%, 92%, 93%, 94%, 95%, 96%, 97%, 98%, 99% or 100% identity) to SEQ ID NO: 676. In some aspects, the VL that specifically binds to cMet comprises an amino acid sequence having at least 80% identity (such as at least 85%, 90%, 91%, 92%, 93%, 94%, 95%, 96%, 97%, 98%, 99% or 100% identity) to SEQ ID NO: 708; and the VH that specifically binds to cMet comprises an amino acid sequence having at least 80% identity (such as at least 85%, 90%, 91%, 92%, 93%, 94%, 95%, 96%, 97%, 98%, 99% or 100% identity) to SEQ ID NO: 712. In some aspects, the VL that specifically binds to CD20 comprises an amino acid sequence having at least 80% identity (such as at least 85%, 90%, 91%, 92%, 93%, 94%, 95%, 96%, 97%, 98%, 99% or 100% identity) to SEQ ID NO: 870 and the VH that specifically binds to CD20 comprises an amino acid sequence having at least 80% identity (such as at least 85%, 90%, 91%, 92%, 93%, 94%, 95%, 96%, 97%, 98%, 99% or 100% identity) to SEQ ID NO: 866. In some aspects, the VL that specifically binds to CD19 comprises an amino acid sequence having at least 80% identity (such as at least 85%, 90%, 91%, 92%, 93%, 94%, 95%, 96%, 97%, 98%, 99% or 100% identity) to DIQLTQSPASLAVSLGQRATISCKASQSVDYDGDSYLNWYQQIPGQPPKLLIYDASNLVS GIPPRFSGSGSGTDFTLNIHPVEKVDAATYHCQQSTEDPWTFGGGTKLEIK (SEQ ID NO: 882) and the VH that specifically binds to CD19 comprises an amino acid sequence having at least 80% identity (such as at least 85%, 90%, 91%, 92%, 93%, 94%, 95%, 96%, 97%, 98%, 99% or 100% identity) to

(SEQ ID NO: 883)
QVQLQQSGAELVRPGSSVKISCKASGYAFSSYWMNWV
KQRPGQGLEWIGQIWPGDGDTNYNGKFKGKATLTADE
SSSTAYMQLSSLASEDSAVYFCARRETTTVGRYYYAM
DYWGQGTTVTVSS.

In some aspects, the antigen binding polypeptide complex (e.g., antibody or antigen binding fragment thereof) of the invention specifically binds to CD3. In some aspects, the antigen binding polypeptide complex specifically binds to CD3 and one or more TAAs, such as, e.g., A2AR, APRIL, ATPDase, BAFF, BAFFR, BCMA, BlyS, BTK, BTLA, B7DC, B7H1, B7H2, B7H3, B7H4, B7H5, B7H6, B7H7, B7RP1, B7-4, C3, C5, CCL2, CCL3, CCL4, CCL5, CCL7, CCL8, CCL11, CCL15, CCL17, CCL19, CCL20, CCL21, CCL25, CCR3, CCR4, CD16A, CD19, CD20, CD24, CD27, CD28, CD30, CD38, CD39, CD40, CD40L, CD47, CD52, CD70, CD80, CD86, CD123, CD133, CD137, CD137L, CD160, CD272, CEACAM5, CLEC9, CLEC91, CRTH2, CSF-1, CSF-2, CSF-3, CXCL1, CXCL2, CXCL4, CXCL12, CXCL13, CXCR3, cMet, CTLA4, DLL3, DLL4, DNGR-1, E-cadherin, EGFR, ENTPD1, EpCAM, FCER1, FCER1A, FCER2, FGFR, FLAP, FOLH1, Gi24, GITR, GITRL, GPR5, GP100, GPRC5D, HER2, HER3, ICOSL, ICOS, HHLA2, HMGB1, HVEM, IDO, IFNa, IgE, IGF1R, IL2Rbeta, IL1, ILIA, IL1B, IL1F10, IL2, IL4, IL4Ra, IL5, IL5R, IL6, IL7, IL7Ra, IL8, IL9, IL9R, IL10, rhILlO, IL12, IL13, IL13Ra1, IL13Ra2, IL15, IL17, IL17Rb, IL18, IL22, IL23, IL25, IL7, IL33, IL35, ITGB4, ITK, KIR, LAG3, LAMP1, leptin, LPFS2, MHC class II, MUC-1, MUC-16, NCR3LG1, NKG2D, NKp46, NTPDase-1, OX40, OX40L, PD-1, PD-L1, PD-L2, PROM1, S152, SIRPalpha, SISP1, SLC, SPG64, ST2, STEAP1, STEAP2, Syk kinase, STEAP1, TROP2, TACI, TDO, TGFBETA, T14, TIGIT, TIM3, TLR, TLR2, TLR4, TLR5, TLR9, TMEF1, TNFa, TNFRSF7, Tp55, TREM1, TSLP, TSLPR, TWEAK, VEGF, VISTA, Vstm3, or WUCAM. For example, the antigen binding polypeptide complex may specifically bind CD3, CD19 and CD20. For example, the antigen binding polypeptide complex may specifically bind CD3, cMet and Trop2.

Antigen binding sequences (e.g., CDR, VH, VL, heavy chain and light chain sequences from antibodies) for A2AR, APRIL, ATPDase, BAFF, BAFFR, BCMA, BlyS, BTK, BTLA, B7DC, B7H1, B7H2, B7H3, B7H4, B7H5, B7H6, B7H7, B7RP1, B7-4, C3, C5, CCL2, CCL3, CCL4, CCL5, CCL7, CCL8, CCL11, CCL15, CCL17, CCL19, CCL20, CCL21, CCL25, CCR3, CCR4, CD16A, CD19, CD20, CD24, CD27, CD28, CD30, CD38, CD39, CD40, CD40L, CD47, CD52, CD70, CD80, CD86, CD123, CD133, CD137, CD137L, CD160, CD272, CEACAM5, CLEC9, CLEC91, CRTH2, CSF-1, CSF-2, CSF-3, CXCL1, CXCL2, CXCL4, CXCL12, CXCL13, CXCR3, cMet, CTLA4, DLL3, DLL4, DNGR-1, E-cadherin, EGFR, ENTPD1, EpCAM, FCER1, FCER1A, FCER2, FGFR, FLAP, FOLH1, Gi24, GITR, GITRL, GPR5, GP100, GPRC5D, HER2, HER3, ICOSL, ICOS, HHLA2, HMGB1, HVEM, IDO, IFNa, IgE, IGF1R, IL2Rbeta, IL1, ILIA, IL1B, IL1F10, IL2, IL4, IL4Ra, IL5, IL5R, IL6, IL7, IL7Ra, IL8, IL9, IL9R, IL10, rhILlO, IL12, IL13, IL13Ra1, IL13Ra2, IL15, IL17, IL17Rb, IL18, IL22, IL23, IL25, IL7, IL33, IL35, ITGB4, ITK, KIR, LAG3, LAMP1, leptin, LPFS2, MHC class II, MUC-1, MUC-16, NCR3LG1, NKG2D, NKp46, NTPDase-1, OX40, OX40L, PD-1, PD-L1, PD-L2, PROM1, S152, SIRPalpha, SISP1, SLC, SPG64, ST2, STEAP1, STEAP2, Syk kinase, STEAP1, TROP2, TACI, TDO, TGFBETA, T14, TIGIT, TIM3, TLR, TLR2, TLR4, TLR5, TLR9, TMEF1, TNFa, TNFRSF7, Tp55, TREM1, TSLP, TSLPR, TWEAK, VEGF, VISTA, Vstm3, or WUCAM are well known. For example, sequences for CD3, CD19, CD28, and CD38 are well known and include, but are not limited to, GenBank Accession Nos. AAA39272.1, AAA39159.1, ABN79462.1, AVW80143.1, AVW80142.1, AVW80141.1, AAB34430.1, AAB34429.1, CAD45042.1, 4CMH_C and 4CMH_B. Such sequences are also described, for example, in Wernly et al., Cells, 9(2):295, 2020; Arakawa et al., Journal of Biochemistry, 120(3):657-662, 1996; Cole et al., Transplantation, 68(4):563-571, 1999; Li et al., International Immunopharmacology, 62:299-308, 2018; Castella et al., Methods & Clinical Development, 12:134-144, 2019; Sun et al., Molecular Immunology, 41(9):929-938, 2004; Iwaszkiewicz-Grzes et al., Cytotherapy, 22(11):629-641, 2020, Rosinski et al., Transplant Direct, 1(2):e7, 2015; Ellis et al., J Immunology, 155(2):925-937, 1995; Stevenson et al., Blood, 77(5):1071-1079, 1991; Chillemi et al., Molecular Medicine, 19:99-108, 2013, and Int'l Pub. No. WO 2020/076853.

47) In addition, molecular biology and recombinant DNA methods for making, screening and engineering antigen binding complexes and antibodies containing such sequences are well known and described, for example, in Adair et al. Human Antibodies, 5(1-2):41-47, 1994; Kostelny et al., J Immunol, 148(5):1547-1553 (1992), Shiraiwa et al., Methods, 154:10-20, 2019; and Zola, “Monoclonal Antibodies: A Manual of Techniques,” 1987, 1^{st} Ed., CRC Press; and Steinitz, Human Antibodies, 18(1-2):1-10, 2009.

In some aspects, a VL and a corresponding VH of the antigen binding polypeptide complex specifically bind to CD3. In some aspects, one VL and one corresponding VH of the antigen binding polypeptide complex specifically bind to CD3. In some aspects, VL3 and VH3 specifically bind to CD3. In some aspects, VL3 comprises a CDR1 comprising an amino acid sequence having at least 90% identity, at least 95% identity, or 100% identity to any one of SEQ ID NOs:35, 44, 99, 446, 456, 466, 476, 520, 663, 699, 735, 763, 799, 835, 855 and 863; a CDR2 comprising an amino acid sequence having at least 90% identity, at least 95% identity, or 100% identity to any one of SEQ ID NOs:36, 45, 100, 447 (DT), 457 (DT), 467 (DT), 477 (DT), 521, 664, 700, 736, 764, 800, 836, 856 and 864; and a CDR3 comprising an amino acid sequence having at least 90% identity, at least 95% identity, or 100% identity to any one of SEQ ID NOs:37, 46, 101, 448, 458, 468, 478, 522, 665, 701, 737, 765, 801, 837, 857 and 865; and VH3 comprises a CDR1 comprising an amino acid sequence having at least 90% identity, at least 95% identity, or 100% identity to any one of SEQ ID NOs:32, 40, 96, 433, 524, 667, 703, 739, 767, 803. 839, 851 and 859; a CDR2 comprising an amino acid sequence having at least 90% identity, at least 95% identity, or 100% identity to any one of SEQ ID NO:33, 41, 97, 434, 525, 668, 704, 740, 768, 804, 840, 852 and 860; and a CDR3 comprising an amino acid sequence having at least 90% identity, at least 95% identity, or 100% identity to any one of SEQ ID NOs:34, 42, 98, 435, 526, 669, 705, 741, 769, 805, 841, 853 and 861. In some aspects, VL3 comprises a CDR1 comprising an amino acid sequence having at least 90% identity to SEQ ID NO:35; a CDR2 comprising an amino acid sequence having at least 90% identity to SEQ ID NO:36; and a CDR3 comprising an amino acid sequence having at least 90% identity to SEQ ID NO:37; and VH3 comprises a CDR1 comprising an amino acid sequence having at least 90% identity to SEQ ID NO:32; a CDR2 comprising an amino acid sequence having at least 90% identity to SEQ ID NO:33; and a CDR3 comprising an amino acid sequence having at least 90% identity to SEQ ID NO:34. In some aspects, VL3 comprises a CDR1 comprising an amino acid sequence having at least 90% identity to SEQ ID NO:44; a CDR2 comprising an amino acid sequence having at least 90% identity to SEQ ID NO:45; and a CDR3 comprising an amino acid sequence having at least 90% identity to SEQ ID NO:46; and VH3 comprises a CDR1 comprising an amino acid sequence having at least 90% identity to SEQ ID NO:40; a CDR2 comprising an amino acid sequence having at least 90% identity to SEQ ID NO:41; and a CDR3 comprising an amino acid sequence having at least 90% identity to SEQ ID NO:42. In some aspects, VL3 comprises a CDR1 comprising an amino acid sequence having at least 90% identity to SEQ ID NO:446; a CDR2 comprising an amino acid sequence having at least 90% identity to SEQ ID NO:447 (DT); and a CDR3 comprising an amino acid sequence having at least 90% identity to SEQ ID NO:448; and VH3 comprises a CDR1 comprising an amino acid sequence having at least 90% identity to SEQ ID NO:433; a CDR2 comprising an amino acid sequence having at least 90% identity to SEQ ID NO:434; and a CDR3 comprising an amino acid sequence having at least 90% identity to SEQ ID NO:435. In some aspects, VL3 comprises a CDR1 comprising an amino acid sequence having at least 90% identity to SEQ ID NO:456; a CDR2 comprising an amino acid sequence having at least 90% identity to SEQ ID NO:457 (DT); and a CDR3 comprising an amino acid sequence having at least 90% identity to SEQ ID NO:458; and VH3 comprises a CDR1 comprising an amino acid sequence having at least 90% identity to SEQ ID NO:433; a CDR2 comprising an amino acid sequence having at least 90% identity to SEQ ID NO:434; and a CDR3 comprising an amino acid sequence having at least 90% identity to SEQ ID NO:435. In some aspects, VL3 comprises a CDR1 comprising an amino acid sequence having at least 90% identity to SEQ ID NO:466; a CDR2 comprising an amino acid sequence having at least 90% identity to SEQ ID NO:467 (DT); and a CDR3 comprising an amino acid sequence having at least 90% identity to SEQ ID NO:468; and VH3 comprises a CDR1 comprising an amino acid sequence having at least 90% identity to SEQ ID NO:433; a CDR2 comprising an amino acid sequence having at least 90% identity to SEQ ID NO:434; and a CDR3 comprising an amino acid sequence having at least 90% identity to SEQ ID NO:435. In some aspects, VL3 comprises a CDR1 comprising an amino acid sequence having at least 90% identity to SEQ ID NO:476; a CDR2 comprising an amino acid sequence having at least 90% identity to SEQ ID NO:477 (DT); and a CDR3 comprising an amino acid sequence having at least 90% identity to SEQ ID NO:478; and VH3 comprises a CDR1 comprising an amino acid sequence having at least 90% identity to SEQ ID NO:433; a CDR2 comprising an amino acid sequence having at least 90% identity to SEQ ID NO:434; and a CDR3 comprising an amino acid sequence having at least 90% identity to SEQ ID NO:435. In some aspects, VL3 comprises a CDR1 comprising an amino acid sequence having at least 90% identity to SEQ ID NO:520; a CDR2 comprising an amino acid sequence having at least 90% identity to SEQ ID NO:521; and a CDR3 comprising an amino acid sequence having at least 90% identity to SEQ ID NO:522; and VH3 comprises a CDR1 comprising an amino acid sequence having at least 90% identity to SEQ ID NO:524; a CDR2 comprising an amino acid sequence having at least 90% identity to SEQ ID NO:525; and a CDR3 comprising an amino acid sequence having at least 90% identity to SEQ ID NO:526. In some aspects, VL3 comprises a CDR1 comprising an amino acid sequence having at least 90% identity to SEQ ID NO:663; a CDR2 comprising an amino acid sequence having at least 90% identity to SEQ ID NO:664; and a CDR3 comprising an amino acid sequence having at least 90% identity to SEQ ID NO:665; and VH3 comprises a CDR1 comprising an amino acid sequence having at least 90% identity to SEQ ID NO:667; a CDR2 comprising an amino acid sequence having at least 90% identity to SEQ ID NO:668; and a CDR3 comprising an amino acid sequence having at least 90% identity to SEQ ID NO:669. In some aspects, VL3 comprises a CDR1 comprising an amino acid sequence having at least 90% identity to SEQ ID NO:699; a CDR2 comprising an amino acid sequence having at least 90% identity to SEQ ID NO: 700; and a CDR3 comprising an amino acid sequence having at least 90% identity to SEQ ID NO:701; and VH3 comprises a CDR1 comprising an amino acid sequence having at least 90% identity to SEQ ID NO:703; a CDR2 comprising an amino acid sequence having at least 90% identity to SEQ ID NO:704; and a CDR3 comprising an amino acid sequence having at least 90% identity to SEQ ID NO:705. In some aspects, VL3 comprises a CDR1 comprising an amino acid sequence having at least 90% identity to SEQ ID NO:735; a CDR2 comprising an amino acid sequence having at least 90% identity to SEQ ID NO:736; and a CDR3 comprising an amino acid sequence having at least 90% identity to SEQ ID NO:737 and VH3 comprises a CDR1 comprising an amino acid sequence having at least 90% identity to SEQ ID NO:739; a CDR2 comprising an amino acid sequence having at least 90% identity to SEQ ID NO:740; and a CDR3 comprising an amino acid sequence having at least 90% identity to SEQ ID NO:741. In some aspects, VL3 comprises a CDR1 comprising an amino acid sequence having at least 90% identity to SEQ ID NO:763; a CDR2 comprising an amino acid sequence having at least 90% identity to SEQ ID NO:764; and a CDR3 comprising an amino acid sequence having at least 90% identity to SEQ ID NO:765; and VH3 comprises a CDR1 comprising an amino acid sequence having at least 90% identity to SEQ ID NO:767; a CDR2 comprising an amino acid sequence having at least 90% identity to SEQ ID NO:768; and a CDR3 comprising an amino acid sequence having at least 90% identity to SEQ ID NO:769. In some aspects, VL3 comprises a CDR1 comprising an amino acid sequence having at least 90% identity to SEQ ID NO:799; a CDR2 comprising an amino acid sequence having at least 90% identity to SEQ ID NO:800; and a CDR3 comprising an amino acid sequence having at least 90% identity to SEQ ID NO:801; and VH3 comprises a CDR1 comprising an amino acid sequence having at least 90% identity to SEQ ID NO:803; a CDR2 comprising an amino acid sequence having at least 90% identity to SEQ ID NO:804; and a CDR3 comprising an amino acid sequence having at least 90% identity to SEQ ID NO:805. In some aspects, VL3 comprises a CDR1 comprising an amino acid sequence having at least 90% identity to SEQ ID NO:835; a CDR2 comprising an amino acid sequence having at least 90% identity to SEQ ID NO:836 and a CDR3 comprising an amino acid sequence having at least 90% identity to SEQ ID NO:837; and VH3 comprises a CDR1 comprising an amino acid sequence having at least 90% identity to SEQ ID NO:839; a CDR2 comprising an amino acid sequence having at least 90% identity to SEQ ID NO: 840; and a CDR3 comprising an amino acid sequence having at least 90% identity to SEQ ID NO:841. In some aspects, VL3 comprises a CDR1 comprising an amino acid sequence having at least 90% identity to SEQ ID NO:855; a CDR2 comprising an amino acid sequence having at least 90% identity to SEQ ID NO:856; and a CDR3 comprising an amino acid sequence having at least 90% identity to SEQ ID NO:857; and VH3 comprises a CDR1 comprising an amino acid sequence having at least 90% identity to SEQ ID NO:851; a CDR2 comprising an amino acid sequence having at least 90% identity to SEQ ID NO:852; and a CDR3 comprising an amino acid sequence having at least 90% identity to SEQ ID NO:853. In some aspects, VL3 comprises a CDR1 comprising an amino acid sequence having at least 90% identity to SEQ ID NO:863; a CDR2 comprising an amino acid sequence having at least 90% identity to SEQ ID NO: 864; and a CDR3 comprising an amino acid sequence having at least 90% identity to SEQ ID NO:865; and VH3 comprises a CDR1 comprising an amino acid sequence having at least 90% identity to SEQ ID NO:859 a CDR2 comprising an amino acid sequence having at least 90% identity to SEQ ID NO: 860; and a CDR3 comprising an amino acid sequence having at least 90% identity to SEQ ID NO:861. In some aspects, VL3 comprises a CDR1 comprising an amino acid sequence having at least 90% identity to SEQ ID NO:99; a CDR2 comprising an amino acid sequence having at least 90% identity to SEQ ID NO:100; and a CDR3 comprising an amino acid sequence having at least 90% identity to SEQ ID NO:100; and VH3 comprises a CDR1 comprising an amino acid sequence having at least 90% identity to SEQ ID NO:96 a CDR2 comprising an amino acid sequence having at least 90% identity to SEQ ID NO:97; and a CDR3 comprising an amino acid sequence having at least 90% identity to SEQ ID NO:98. As used herein, “at least 90% identity” includes at least 91%, 92%, 93%, 94%, 95%, 96%, 97%, 98%, 99% and 100% identity to the recited reference sequence. In some aspects, VL3 comprises a CDR1 comprising the amino acid sequence of SEQ ID NO:35; a CDR2 comprising the amino acid sequence of SEQ ID NO:36; and a CDR3 comprising the amino acid sequence of SEQ ID NO:37; and VH3 comprises a CDR1 comprising the amino acid sequence of SEQ ID NO:32; a CDR2 comprising the amino acid sequence of SEQ ID NO:33; and a CDR3 comprising the amino acid sequence of SEQ ID NO:34. In some aspects, VL3 comprises a CDR1 comprising the amino acid sequence of SEQ ID NO:44; a CDR2 comprising the amino acid sequence of SEQ ID NO:45; and a CDR3 comprising the amino acid sequence of SEQ ID NO:46; and VH3 comprises a CDR1 comprising the amino acid sequence of SEQ ID NO:40; a CDR2 comprising the amino acid sequence of SEQ ID NO:41; and a CDR3 comprising the amino acid sequence of SEQ ID NO:42. In some aspects, VL3 comprises a CDR1 comprising the amino acid sequence of SEQ ID NO:446; a CDR2 comprising the amino acid sequence of SEQ ID NO:447 (DT); and a CDR3 comprising the amino acid sequence of SEQ ID NO:448; and VH3 comprises a CDR1 comprising the amino acid sequence of SEQ ID NO:433; a CDR2 comprising the amino acid sequence of SEQ ID NO:434; and a CDR3 comprising the amino acid sequence of SEQ ID NO:435. In some aspects, VL3 comprises a CDR1 comprising the amino acid sequence of SEQ ID NO:456; a CDR2 comprising the amino acid sequence of SEQ ID NO:457 (DT); and a CDR3 comprising the amino acid sequence of SEQ ID NO:458; and VH3 comprises a CDR1 comprising the amino acid sequence of SEQ ID NO:433; a CDR2 comprising the amino acid sequence of SEQ ID NO:434; and a CDR3 comprising the amino acid sequence of SEQ ID NO:435. In some aspects, VL3 comprises a CDR1 comprising the amino acid sequence of SEQ ID NO:466; a CDR2 comprising the amino acid sequence of SEQ ID NO:467 (DT); and a CDR3 comprising the amino acid sequence of SEQ ID NO:468; and VH3 comprises a CDR1 comprising the amino acid sequence of SEQ ID NO:433; a CDR2 comprising the amino acid sequence of SEQ ID NO:434; and a CDR3 comprising the amino acid sequence of SEQ ID NO:435. In some aspects, VL3 comprises a CDR1 comprising the amino acid sequence of SEQ ID NO:476; a CDR2 comprising the amino acid sequence of SEQ ID NO:477 (DT); and a CDR3 comprising the amino acid sequence of SEQ ID NO:478; and VH3 comprises a CDR1 the amino acid sequence of SEQ ID NO:433; a CDR2 comprising the amino acid sequence of SEQ ID NO:434; and a CDR3 comprising the amino acid sequence of SEQ ID NO:435. In some aspects, VL3 comprises a CDR1 comprising the amino acid sequence of SEQ ID NO:520; a CDR2 comprising the amino acid sequence of SEQ ID NO:521; and a CDR3 comprising the amino acid sequence of SEQ ID NO:522; and VH3 comprises a CDR1 comprising the amino acid sequence of SEQ ID NO:524; a CDR2 comprising the amino acid sequence of SEQ ID NO:525; and a CDR3 comprising the amino acid sequence of SEQ ID NO:526. In some aspects, VL3 comprises a CDR1 comprising the amino acid sequence of SEQ ID NO:663; a CDR2 comprising the amino acid sequence of SEQ ID NO:664; and a CDR3 comprising the amino acid sequence of SEQ ID NO:665; and VH3 comprises a CDR1 comprising the amino acid sequence of SEQ ID NO:667; a CDR2 comprising the amino acid sequence of SEQ ID NO:668; and a CDR3 comprising the amino acid sequence of SEQ ID NO:669. In some aspects, VL3 comprises a CDR1 comprising the amino acid sequence of SEQ ID NO:699; a CDR2 comprising the amino acid sequence of SEQ ID NO: 700; and a CDR3 comprising the amino acid sequence of SEQ ID NO:701; and VH3 comprises a CDR1 comprising the amino acid sequence of SEQ ID NO:703; a CDR2 comprising the amino acid sequence of SEQ ID NO:704; and a CDR3 comprising the amino acid sequence of SEQ ID NO:705. In some aspects, VL3 comprises a CDR1 comprising the amino acid sequence of SEQ ID NO:735; a CDR2 comprising the amino acid sequence of SEQ ID NO:736; and a CDR3 comprising the amino acid sequence of SEQ ID NO:737 and VH3 comprises a CDR1 comprising the amino acid sequence of SEQ ID NO:739; a CDR2 comprising the amino acid sequence of SEQ ID NO:740; and a CDR3 comprising the amino acid sequence of SEQ ID NO:741. In some aspects, VL3 comprises a CDR1 comprising the amino acid sequence of SEQ ID NO:763; a CDR2 comprising the amino acid sequence of SEQ ID NO:764; and a CDR3 comprising the amino acid sequence of SEQ ID NO:765; and VH3 comprises a CDR1 comprising the amino acid sequence of SEQ ID NO:767; a CDR2 the amino acid sequence of SEQ ID NO:768; and a CDR3 comprising the amino acid sequence of SEQ ID NO:769. In some aspects, VL3 comprises a CDR1 comprising the amino acid sequence of SEQ ID NO:799; a CDR2 comprising the amino acid sequence of SEQ ID NO:800; and a CDR3 comprising the amino acid sequence of SEQ ID NO:801; and VH3 comprises a CDR1 comprising an amino acid sequence having at least 90% identity to SEQ ID NO:803; a CDR2 comprising the amino acid sequence of SEQ ID NO:804; and a CDR3 comprising the amino acid sequence of SEQ ID NO:805. In some aspects, VL3 comprises a CDR1 comprising the amino acid sequence of SEQ ID NO:835; a CDR2 comprising the amino acid sequence of SEQ ID NO:836 and a CDR3 comprising the amino acid sequence of SEQ ID NO:837; and VH3 comprises a CDR1 comprising the amino acid sequence of SEQ ID NO:839; a CDR2 comprising the amino acid sequence of SEQ ID NO:840; and a CDR3 comprising the amino acid sequence of SEQ ID NO:841. In some aspects, VL3 comprises a CDR1 comprising the amino acid sequence of SEQ ID NO:855; a CDR2 comprising the amino acid sequence of SEQ ID NO:856; and a CDR3 comprising the amino acid sequence of SEQ ID NO:857; and VH3 comprises a CDR1 comprising the amino acid sequence of SEQ ID NO:851; a CDR2 comprising the amino acid sequence of SEQ ID NO:852; and a CDR3 comprising the amino acid sequence of SEQ ID NO:853. In some aspects, VL3 comprises a CDR1 comprising the amino acid sequence of SEQ ID NO:863; a CDR2 comprising the amino acid sequence of SEQ ID NO: 864; and a CDR3 comprising the amino acid sequence of SEQ ID NO:865; and VH3 comprises a CDR1 comprising the amino acid sequence of SEQ ID NO:859 a CDR2 comprising the amino acid sequence of SEQ ID NO:860; and a CDR3 comprising the amino acid sequence of SEQ ID NO:861. In some aspects, VL3 comprises a CDR1 comprising the amino acid sequence of SEQ ID NO:99; a CDR2 comprising the amino acid sequence of SEQ ID NO:100; and a CDR3 comprising the amino acid sequence of SEQ ID NO:101; and VH3 comprises a CDR1 comprising the amino acid sequence of SEQ ID NO:96; a CDR2 comprising the amino acid sequence of SEQ ID NO:97; and a CDR3 comprising the amino acid sequence of SEQ ID NO:98. As used herein, “at least 90% identity” includes at least 91%, 92%, 93%, 94%, 95%, 96%, 97%, 98%, 99% and 100% identity to the recited reference sequence. In some aspects, VL3 comprises a CDR1 comprising the amino acid sequence of SEQ ID NO:35; a CDR2 comprising the amino acid sequence of SEQ ID NO:36; and a CDR3 comprising the amino acid sequence of SEQ ID NO:37; and VH3 comprises a CDR1 comprising the amino acid sequence of SEQ ID NO:32; a CDR2 comprising the amino acid sequence of SEQ ID NO:33; and a CDR3 comprising the amino acid sequence of SEQ ID NO:34. In some aspects, VL3 comprises a CDR1 comprising the amino acid sequence of SEQ ID NO:44; a CDR2 comprising the amino acid sequence of SEQ ID NO:45; and a CDR3 comprising the amino acid sequence of SEQ ID NO:46; and VH3 comprises a CDR1 comprising the amino acid sequence of SEQ ID NO:40; a CDR2 comprising the amino acid sequence of SEQ ID NO:41; and a CDR3 comprising the amino acid sequence of SEQ ID NO:42. In some aspects, VL3 comprises an amino acid sequence having at least 80%, at least 85%, at least 90%, at least 95%, or 100% identity to any one of SEQ ID NOs:39, 47, 103, 422, 445, 455, 465, 475, 519, 662, 698, 734, 762, 798, 834, 854 and 862; and/or VH3 comprises an amino acid sequence having at least 80%, at least 85%, at least 90%, at least 95%, or 100% identity to any one of SEQ ID NOs:38, 43, 102, 423, 432, 523, 666, 702, 738, 766, 802, 838, 850 and 858. In some aspects, VL3 comprises an amino acid sequence having at least 80% identity (such as at least 85%, 90%, 91%, 92%, 93%, 94%, 95%, 96%, 97%, 98%, 99% or 100% identity) to SEQ ID NO:39; and/or VH3 comprises an amino acid sequence having at least 80% identity (such as at least 85%, 90%, 91%, 92%, 93%, 94%, 95%, 96%, 97%, 98%, 99% or 100% identity) to SEQ ID NO:38. In some aspects, VL3 comprises an amino acid sequence having at least 80% identity (such as at least 85%, 90%, 91%, 92%, 93%, 94%, 95%, 96%, 97%, 98%, 99% or 100% identity) to SEQ ID NO:47; and/or VH3 comprises an amino acid sequence having at least 80% identity (such as at least 85%, 90%, 91%, 92%, 93%, 94%, 95%, 96%, 97%, 98%, 99% or 100% identity) to SEQ ID NO:43. In some aspects, VL3 comprises an amino acid sequence having at least 80% identity (such as at least 85%, 90%, 91%, 92%, 93%, 94%, 95%, 96%, 97%, 98%, 99% or 100% identity) to SEQ ID NO:39; and/or VH3 comprises an amino acid sequence having at least 80% identity (such as at least 85%, 90%, 91%, 92%, 93%, 94%, 95%, 96%, 97%, 98%, 99% or 100% identity) to SEQ ID NO:43. In some aspects, VL3 comprises an amino acid sequence having at least 80% identity (such as at least 85%, 90%, 91%, 92%, 93%, 94%, 95%, 96%, 97%, 98%, 99% or 100% identity) to SEQ ID NO:47; and/or VH3 comprises an amino acid sequence having at least 80% identity (such as at least 85%, 90%, 91%, 92%, 93%, 94%, 95%, 96%, 97%, 98%, 99% or 100% identity) to SEQ ID NO:38. In some aspects, VL3 comprises an amino acid sequence having at least 80% identity (such as at least 85%, 90%, 91%, 92%, 93%, 94%, 95%, 96%, 97%, 98%, 99% or 100% identity) to SEQ ID NO:422; and/or VH3 comprises an amino acid sequence having at least 80% identity (such as at least 85%, 90%, 91%, 92%, 93%, 94%, 95%, 96%, 97%, 98%, 99% or 100% identity) to SEQ ID NO:423. In some aspects, VL3 comprises an amino acid sequence having at least 80% identity (such as at least 85%, 90%, 91%, 92%, 93%, 94%, 95%, 96%, 97%, 98%, 99% or 100% identity) to SEQ ID NO:445; and/or VH3 comprises an amino acid sequence having at least 80% identity (such as at least 85%, 90%, 91%, 92%, 93%, 94%, 95%, 96%, 97%, 98%, 99% or 100% identity) to SEQ ID NO:432. In some aspects, VL3 comprises an amino acid sequence having at least 80% identity (such as at least 85%, 90%, 91%, 92%, 93%, 94%, 95%, 96%, 97%, 98%, 99% or 100% identity) to SEQ ID NO:455; and/or VH3 comprises an amino acid sequence having at least 80% identity (such as at least 85%, 90%, 91%, 92%, 93%, 94%, 95%, 96%, 97%, 98%, 99% or 100% identity) to SEQ ID NO:432. In some aspects, VL3 comprises an amino acid sequence having at least 80% identity (such as at least 85%, 90%, 91%, 92%, 93%, 94%, 95%, 96%, 97%, 98%, 99% or 100% identity) to SEQ ID NO:465; and/or VH3 comprises an amino acid sequence having at least 80% identity (such as at least 85%, 90%, 91%, 92%, 93%, 94%, 95%, 96%, 97%, 98%, 99% or 100% identity) to SEQ ID NO:432. In some aspects, VL3 comprises an amino acid sequence having at least 80% identity (such as at least 85%, 90%, 91%, 92%, 93%, 94%, 95%, 96%, 97%, 98%, 99% or 100% identity) to SEQ ID NO:475; and/or VH3 comprises an amino acid sequence having at least 80% identity (such as at least 85%, 90%, 91%, 92%, 93%, 94%, 95%, 96%, 97%, 98%, 99% or 100% identity) to SEQ ID NO:432. In some aspects, VL3 comprises an amino acid sequence having at least 80% identity (such as at least 85%, 90%, 91%, 92%, 93%, 94%, 95%, 96%, 97%, 98%, 99% or 100% identity) to SEQ ID NO:519; and/or VH3 comprises an amino acid sequence having at least 80% identity (such as at least 85%, 90%, 91%, 92%, 93%, 94%, 95%, 96%, 97%, 98%, 99% or 100% identity) to SEQ ID NO:523. In some aspects, VL3 comprises an amino acid sequence having at least 80% identity (such as at least 85%, 90%, 91%, 92%, 93%, 94%, 95%, 96%, 97%, 98%, 99% or 100% identity) to SEQ ID NO:662; and/or VH3 comprises an amino acid sequence having at least 80% identity (such as at least 85%, 90%, 91%, 92%, 93%, 94%, 95%, 96%, 97%, 98%, 99% or 100% identity) to SEQ ID NO:666. In some aspects, VL3 comprises an amino acid sequence having at least 80% identity (such as at least 85%, 90%, 91%, 92%, 93%, 94%, 95%, 96%, 97%, 98%, 99% or 100% identity) to SEQ ID NO:698; and/or VH3 comprises an amino acid sequence having at least 80% identity (such as at least 85%, 90%, 91%, 92%, 93%, 94%, 95%, 96%, 97%, 98%, 99% or 100% identity) to SEQ ID NO:702. In some aspects, VL3 comprises an amino acid sequence having at least 80% identity (such as at least 85%, 90%, 91%, 92%, 93%, 94%, 95%, 96%, 97%, 98%, 99% or 100% identity) to SEQ ID NO:734; and/or VH3 comprises an amino acid sequence having at least 80% identity (such as at least 85%, 90%, 91%, 92%, 93%, 94%, 95%, 96%, 97%, 98%, 99% or 100% identity) to SEQ ID NO:738. In some aspects, VL3 comprises an amino acid sequence having at least 80% identity (such as at least 85%, 90%, 91%, 92%, 93%, 94%, 95%, 96%, 97%, 98%, 99% or 100% identity) to SEQ ID NO:762; and/or VH3 comprises an amino acid sequence having at least 80% identity (such as at least 85%, 90%, 91%, 92%, 93%, 94%, 95%, 96%, 97%, 98%, 99% or 100% identity) to SEQ ID NO:766. In some aspects, VL3 comprises an amino acid sequence having at least 80% identity (such as at least 85%, 90%, 91%, 92%, 93%, 94%, 95%, 96%, 97%, 98%, 99% or 100% identity) to SEQ ID NO:798; and/or VH3 comprises an amino acid sequence having at least 80% identity (such as at least 85%, 90%, 91%, 92%, 93%, 94%, 95%, 96%, 97%, 98%, 99% or 100% identity) to SEQ ID NO:802. In some aspects, VL3 comprises an amino acid sequence having at least 80% identity (such as at least 85%, 90%, 91%, 92%, 93%, 94%, 95%, 96%, 97%, 98%, 99% or 100% identity) to SEQ ID NO:834; and/or VH3 comprises an amino acid sequence having at least 80% identity (such as at least 85%, 90%, 91%, 92%, 93%, 94%, 95%, 96%, 97%, 98%, 99% or 100% identity) to SEQ ID NO:838. In some aspects, VL3 comprises an amino acid sequence having at least 80% identity (such as at least 85%, 90%, 91%, 92%, 93%, 94%, 95%, 96%, 97%, 98%, 99% or 100% identity) to SEQ ID NO:854; and/or VH3 comprises an amino acid sequence having at least 80% identity (such as at least 85%, 90%, 91%, 92%, 93%, 94%, 95%, 96%, 97%, 98%, 99% or 100% identity) to SEQ ID NO:850. In some aspects, VL3 comprises an amino acid sequence having at least 80% identity (such as at least 85%, 90%, 91%, 92%, 93%, 94%, 95%, 96%, 97%, 98%, 99% or 100% identity) to SEQ ID NO:862; and/or VH3 comprises an amino acid sequence having at least 80% identity (such as at least 85%, 90%, 91%, 92%, 93%, 94%, 95%, 96%, 97%, 98%, 99% or 100% identity) to SEQ ID NO:858.

In some aspects, VL4 and VH4 specifically bind to CD3. In some aspects, VL4 comprises a CDR1 comprising an amino acid sequence having at least 90% identity, at least 95% identity, or 100% identity to any one of SEQ ID NOs:35, 44, 99, 446, 456, 466, 476, 520, 663, 699, 735, 763, 799, 835, 855 and 863; a CDR2 comprising an amino acid sequence having at least 90% identity, at least 95% identity, or 100% identity to any one of SEQ ID NOs:36, 45, 100, 447 (DT), 457 (DT), 467 (DT), 477 (DT), 521, 664, 700, 736, 764, 800, 836, 856 and 864; and a CDR3 comprising an amino acid sequence having at least 90% identity, at least 95% identity, or 100% identity to any one of SEQ ID NOs:37, 46, 101, 448, 458, 468, 478, 522, 665, 701, 737, 765, 801, 837, 857 and 865; and VH4 comprises a CDR1 comprising an amino acid sequence having at least 90% identity, at least 95% identity, or 100% identity to any one of SEQ ID NOs:32, 40, 96, 433, 524, 667, 703, 739, 767, 803. 839, 851 and 859; a CDR2 comprising an amino acid sequence having at least 90% identity, at least 95% identity, or 100% identity to any one of SEQ ID NOs:33, 41, 97, 434, 525, 668, 704, 740, 768, 804, 840, 852 and 860; and a CDR3 comprising an amino acid sequence having at least 90% identity, at least 95% identity, or 100% identity to any one of SEQ ID NOs:34, 42, 98, 435, 526, 669, 705, 741, 769, 805, 841, 853 and 861. In some aspects, VL4 comprises a CDR1 comprising an amino acid sequence having at least 90% identity to SEQ ID NO:35; a CDR2 comprising an amino acid sequence having at least 90% identity to SEQ ID NO:36; and a CDR3 comprising an amino acid sequence having at least 90% identity to SEQ ID NO:37; and VH4 comprises a CDR1 comprising an amino acid sequence having at least 90% identity to SEQ ID NO:32; a CDR2 comprising an amino acid sequence having at least 90% identity to SEQ ID NO:33; and a CDR3 comprising an amino acid sequence having at least 90% identity to SEQ ID NO:34. In some aspects, VL4 comprises a CDR1 comprising an amino acid sequence having at least 90% identity to SEQ ID NO:44; a CDR2 comprising an amino acid sequence having at least 90% identity to SEQ ID NO:45; and a CDR3 comprising an amino acid sequence having at least 90% identity to SEQ ID NO:46; and VH4 comprises a CDR1 comprising an amino acid sequence having at least 90% identity to SEQ ID NO:40; a CDR2 comprising an amino acid sequence having at least 90% identity to SEQ ID NO:41; and a CDR3 comprising an amino acid sequence having at least 90% identity to SEQ ID NO:42. In some aspects, VL4 comprises a CDR1 comprising an amino acid sequence having at least 90% identity to SEQ ID NO:446; a CDR2 comprising an amino acid sequence having at least 90% identity to SEQ ID NO:447 (DT); and a CDR3 comprising an amino acid sequence having at least 90% identity to SEQ ID NO:448; and VH4 comprises a CDR1 comprising an amino acid sequence having at least 90% identity to SEQ ID NO:433; a CDR2 comprising an amino acid sequence having at least 90% identity to SEQ ID NO:434; and a CDR3 comprising an amino acid sequence having at least 90% identity to SEQ ID NO:435. In some aspects, VL4 comprises a CDR1 comprising an amino acid sequence having at least 90% identity to SEQ ID NO:456; a CDR2 comprising an amino acid sequence having at least 90% identity to SEQ ID NO:457 (DT); and a CDR3 comprising an amino acid sequence having at least 90% identity to SEQ ID NO:458; and VH4 comprises a CDR1 comprising an amino acid sequence having at least 90% identity to SEQ ID NO:433; a CDR2 comprising an amino acid sequence having at least 90% identity to SEQ ID NO:434; and a CDR3 comprising an amino acid sequence having at least 90% identity to SEQ ID NO:435. In some aspects, VL4 comprises a CDR1 comprising an amino acid sequence having at least 90% identity to SEQ ID NO:466; a CDR2 comprising an amino acid sequence having at least 90% identity to SEQ ID NO:467 (DT); and a CDR3 comprising an amino acid sequence having at least 90% identity to SEQ ID NO:468; and VH4 comprises a CDR1 comprising an amino acid sequence having at least 90% identity to SEQ ID NO:433; a CDR2 comprising an amino acid sequence having at least 90% identity to SEQ ID NO:434; and a CDR3 comprising an amino acid sequence having at least 90% identity to SEQ ID NO:435. In some aspects, VL4 comprises a CDR1 comprising an amino acid sequence having at least 90% identity to SEQ ID NO:476; a CDR2 comprising an amino acid sequence having at least 90% identity to SEQ ID NO:477 (DT); and a CDR3 comprising an amino acid sequence having at least 90% identity to SEQ ID NO:478; and VH4 comprises a CDR1 comprising an amino acid sequence having at least 90% identity to SEQ ID NO:433; a CDR2 comprising an amino acid sequence having at least 90% identity to SEQ ID NO:434; and a CDR3 comprising an amino acid sequence having at least 90% identity to SEQ ID NO:435. In some aspects, VL4 comprises a CDR1 comprising an amino acid sequence having at least 90% identity to SEQ ID NO:520; a CDR2 comprising an amino acid sequence having at least 90% identity to SEQ ID NO:521; and a CDR3 comprising an amino acid sequence having at least 90% identity to SEQ ID NO:522; and VH4 comprises a CDR1 comprising an amino acid sequence having at least 90% identity to SEQ ID NO:524; a CDR2 comprising an amino acid sequence having at least 90% identity to SEQ ID NO:525; and a CDR3 comprising an amino acid sequence having at least 90% identity to SEQ ID NO:526. In some aspects, VL4 comprises a CDR1 comprising an amino acid sequence having at least 90% identity to SEQ ID NO:663; a CDR2 comprising an amino acid sequence having at least 90% identity to SEQ ID NO:664; and a CDR3 comprising an amino acid sequence having at least 90% identity to SEQ ID NO:665; and VH4 comprises a CDR1 comprising an amino acid sequence having at least 90% identity to SEQ ID NO:667; a CDR2 comprising an amino acid sequence having at least 90% identity to SEQ ID NO:668; and a CDR3 comprising an amino acid sequence having at least 90% identity to SEQ ID NO:669. In some aspects, VL4 comprises a CDR1 comprising an amino acid sequence having at least 90% identity to SEQ ID NO:699; a CDR2 comprising an amino acid sequence having at least 90% identity to SEQ ID NO: 700; and a CDR3 comprising an amino acid sequence having at least 90% identity to SEQ ID NO:701; and VH4 comprises a CDR1 comprising an amino acid sequence having at least 90% identity to SEQ ID NO:703; a CDR2 comprising an amino acid sequence having at least 90% identity to SEQ ID NO:704; and a CDR3 comprising an amino acid sequence having at least 90% identity to SEQ ID NO:705. In some aspects, VL4 comprises a CDR1 comprising an amino acid sequence having at least 90% identity to SEQ ID NO:735; a CDR2 comprising an amino acid sequence having at least 90% identity to SEQ ID NO:736; and a CDR3 comprising an amino acid sequence having at least 90% identity to SEQ ID NO:737 and VH4 comprises a CDR1 comprising an amino acid sequence having at least 90% identity to SEQ ID NO:739; a CDR2 comprising an amino acid sequence having at least 90% identity to SEQ ID NO:740; and a CDR3 comprising an amino acid sequence having at least 90% identity to SEQ ID NO:741. In some aspects, VL4 comprises a CDR1 comprising an amino acid sequence having at least 90% identity to SEQ ID NO:763; a CDR2 comprising an amino acid sequence having at least 90% identity to SEQ ID NO:764; and a CDR3 comprising an amino acid sequence having at least 90% identity to SEQ ID NO:765; and VH4 comprises a CDR1 comprising an amino acid sequence having at least 90% identity to SEQ ID NO:767; a CDR2 comprising an amino acid sequence having at least 90% identity to SEQ ID NO:768; and a CDR3 comprising an amino acid sequence having at least 90% identity to SEQ ID NO:769. In some aspects, VL4 comprises a CDR1 comprising an amino acid sequence having at least 90% identity to SEQ ID NO:799; a CDR2 comprising an amino acid sequence having at least 90% identity to SEQ ID NO:800; and a CDR3 comprising an amino acid sequence having at least 90% identity to SEQ ID NO:801; and VH4 comprises a CDR1 comprising an amino acid sequence having at least 90% identity to SEQ ID NO:803; a CDR2 comprising an amino acid sequence having at least 90% identity to SEQ ID NO: 804; and a CDR3 comprising an amino acid sequence having at least 90% identity to SEQ ID NO:805. In some aspects, VL4 comprises a CDR1 comprising an amino acid sequence having at least 90% identity to SEQ ID NO:835; a CDR2 comprising an amino acid sequence having at least 90% identity to SEQ ID NO:836 and a CDR3 comprising an amino acid sequence having at least 90% identity to SEQ ID NO:837; and VH4 comprises a CDR1 comprising an amino acid sequence having at least 90% identity to SEQ ID NO:839; a CDR2 comprising an amino acid sequence having at least 90% identity to SEQ ID NO:840; and a CDR3 comprising an amino acid sequence having at least 90% identity to SEQ ID NO:841. In some aspects, VL4 comprises a CDR1 comprising an amino acid sequence having at least 90% identity to SEQ ID NO:855; a CDR2 comprising an amino acid sequence having at least 90% identity to SEQ ID NO:856; and a CDR3 comprising an amino acid sequence having at least 90% identity to SEQ ID NO:857; and VH4 comprises a CDR1 comprising an amino acid sequence having at least 90% identity to SEQ ID NO:851; a CDR2 comprising an amino acid sequence having at least 90% identity to SEQ ID NO:852; and a CDR3 comprising an amino acid sequence having at least 90% identity to SEQ ID NO:853. In some aspects, VL4 comprises a CDR1 comprising an amino acid sequence having at least 90% identity to SEQ ID NO:863; a CDR2 comprising an amino acid sequence having at least 90% identity to SEQ ID NO: 864; and a CDR3 comprising an amino acid sequence having at least 90% identity to SEQ ID NO:865; and VH4 comprises a CDR1 comprising an amino acid sequence having at least 90% identity to SEQ ID NO:859 a CDR2 comprising an amino acid sequence having at least 90% identity to SEQ ID NO:860; and a CDR3 comprising an amino acid sequence having at least 90% identity to SEQ ID NO:861. In some aspects, VL4 comprises a CDR1 comprising an amino acid sequence having at least 90% identity to SEQ ID NO:99; a CDR2 comprising an amino acid sequence having at least 90% identity to SEQ ID NO:100; and a CDR3 comprising an amino acid sequence having at least 90% identity to SEQ ID NO:101; and VH4 comprises a CDR1 comprising an amino acid sequence having at least 90% identity to SEQ ID NO:96; a CDR2 comprising an amino acid sequence having at least 90% identity to SEQ ID NO:97; and a CDR3 comprising an amino acid sequence having at least 90% identity to SEQ ID NO:98. As used herein, “at least 90% identity” includes at least 91%, 92%, 93%, 94%, 95%, 96%, 97%, 98%, 99% and 100% identity to the recited reference sequence. In some aspects, VL4 comprises a CDR1 comprising the amino acid sequence of SEQ ID NO:35; a CDR2 comprising the amino acid sequence of SEQ ID NO:36; and a CDR3 comprising the amino acid sequence of SEQ ID NO:37; a″d VH4 comprises a CDR1 comprising the amino acid sequence of SEQ ID NO:32; a CDR2 comprising the amino acid sequence of SEQ ID NO:33; and a CDR3 comprising the amino acid sequence of SEQ ID NO:34. In some aspects, VL4 comprises a CDR1 comprising the amino acid sequence of SEQ ID NO:44; a CDR2 comprising the amino acid sequence of SEQ ID NO:45; and a CDR3 comprising the amino acid sequence of SEQ ID NO:46; and VH4 comprises a CDR1 comprising the amino acid sequence of SEQ ID NO:40; a CDR2 comprising the amino acid sequence of SEQ ID NO:41; and a CDR3 comprising the amino acid sequence of SEQ ID NO:42. In some aspects, VL4 comprises a CDR1 comprising the amino acid sequence of SEQ ID NO:446; a CDR2 comprising the amino acid sequence of SEQ ID NO:447 (DT); and a CDR3 comprising the amino acid sequence of SEQ ID NO:448; and VH4 comprises a CDR1 comprising the amino acid sequence of SEQ ID NO:433; a CDR2 comprising the amino acid sequence of SEQ ID NO:434; and a CDR3 comprising the amino acid sequence of SEQ ID NO:435. In some aspects, VL4 comprises a CDR1 comprising the amino acid sequence of SEQ ID NO:456; a CDR2 comprising the amino acid sequence of SEQ ID NO:457 (DT); and a CDR3 comprising the amino acid sequence of SEQ ID NO:458; and VH4 comprises a CDR1 comprising the amino acid sequence of SEQ ID NO:433; a CDR2 comprising the amino acid sequence of SEQ ID NO:434; and a CDR3 comprising the amino acid sequence of SEQ ID NO:435. In some aspects, VL4 comprises a CDR1 comprising the amino acid sequence of SEQ ID NO:466; a CDR2 comprising the amino acid sequence of SEQ ID NO:467 (DT); and a CDR3 comprising the amino acid sequence of SEQ ID NO:468; and VH4 comprises a CDR1 comprising the amino acid sequence of SEQ ID NO:433; a CDR2 comprising the amino acid sequence of SEQ ID NO:434; and a CDR3 comprising the amino acid sequence of SEQ ID NO:435. In some aspects, VL4 comprises a CDR1 comprising the amino acid sequence of SEQ ID NO:476; a CDR2 comprising the amino acid sequence of SEQ ID NO:477 (DT); and a CDR3 comprising the amino acid sequence of SEQ ID NO:478; and VH4 comprises a CDR1 the amino acid sequence of SEQ ID NO:433; a CDR2 comprising the amino acid sequence of SEQ ID NO:434; and a CDR3 comprising the amino acid sequence of SEQ ID NO:435. In some aspects, VL4 comprises a CDR1 comprising the amino acid sequence of SEQ ID NO:520; a CDR2 comprising the amino acid sequence of SEQ ID NO:521; and a CDR3 comprising the amino acid sequence of SEQ ID NO:522; and VH4 comprises a CDR1 comprising the amino acid sequence of SEQ ID NO:524; a CDR2 comprising the amino acid sequence of SEQ ID NO:525; and a CDR3 comprising the amino acid sequence of SEQ ID NO:526. In some aspects, VL4 comprises a CDR1 comprising the amino acid sequence of SEQ ID NO:663; a CDR2 comprising the amino acid sequence of SEQ ID NO:664; and a CDR3 comprising the amino acid sequence of SEQ ID NO:665; and VH4 comprises a CDR1 comprising the amino acid sequence of SEQ ID NO:667; a CDR2 comprising the amino acid sequence of SEQ ID NO:668; and a CDR3 comprising the amino acid sequence of SEQ ID NO:669. In some aspects, VL4 comprises a CDR1 comprising the amino acid sequence of SEQ ID NO:699; a CDR2 comprising the amino acid sequence of SEQ ID NO: 700; and a CDR3 comprising the amino acid sequence of SEQ ID NO:701; and VH4 comprises a CDR1 comprising the amino acid sequence of SEQ ID NO:703; a CDR2 comprising the amino acid sequence of SEQ ID NO:704; and a CDR3 comprising the amino acid sequence of SEQ ID NO:705. In some aspects, VL4 comprises a CDR1 comprising the amino acid sequence of SEQ ID NO:735; a CDR2 comprising the amino acid sequence of SEQ ID NO:736; and a CDR3 comprising the amino acid sequence of SEQ ID NO:737 and VH4 comprises a CDR1 comprising the amino acid sequence of SEQ ID NO:739; a CDR2 comprising the amino acid sequence of SEQ ID NO:740; and a CDR3 comprising the amino acid sequence of SEQ ID NO:741. In some aspects, VL4 comprises a CDR1 comprising the amino acid sequence of SEQ ID NO:763; a CDR2 comprising the amino acid sequence of SEQ ID NO:764; and a CDR3 comprising the amino acid sequence of SEQ ID NO:765; and VH4 comprises a CDR1 comprising the amino acid sequence of SEQ ID NO:767; a CDR2 the amino acid sequence of SEQ ID NO:768; and a CDR3 comprising the amino acid sequence of SEQ ID NO:769. In some aspects, VL4 comprises a CDR1 comprising the amino acid sequence of SEQ ID NO:799; a CDR2 comprising the amino acid sequence of SEQ ID NO:800; and a CDR3 comprising the amino acid sequence of SEQ ID NO:801; and VH4 comprises a CDR1 comprising an amino acid sequence having at least 90% identity to SEQ ID NO:803; a CDR2 comprising the amino acid sequence of SEQ ID NO:804; and a CDR3 comprising the amino acid sequence of SEQ ID NO:805. In some aspects, VL4 comprises a CDR1 comprising the amino acid sequence of SEQ ID NO:835; a CDR2 comprising the amino acid sequence of SEQ ID NO:836 and a CDR3 comprising the amino acid sequence of SEQ ID NO:837; and VH4 comprises a CDR1 comprising the amino acid sequence of SEQ ID NO:839; a CDR2 comprising the amino acid sequence of SEQ ID NO:840; and a CDR3 comprising the amino acid sequence of SEQ ID NO:841. In some aspects, VL4 comprises a CDR1 comprising the amino acid sequence of SEQ ID NO:855; a CDR2 comprising the amino acid sequence of SEQ ID NO:856; and a CDR3 comprising the amino acid sequence of SEQ ID NO:857; and VH4 comprises a CDR1 comprising the amino acid sequence of SEQ ID NO:851; a CDR2 comprising the amino acid sequence of SEQ ID NO:852; and a CDR3 comprising the amino acid sequence of SEQ ID NO:853. In some aspects, VL4 comprises a CDR1 comprising the amino acid sequence of SEQ ID NO:863; a CDR2 comprising the amino acid sequence of SEQ ID NO: 864; and a CDR3 comprising the amino acid sequence of SEQ ID NO:865; and VH4 comprises a CDR1 comprising the amino acid sequence of SEQ ID NO:859 a CDR2 comprising the amino acid sequence of SEQ ID NO:860; and a CDR3 comprising the amino acid sequence of SEQ ID NO:861. In some aspects, VL4 comprises a CDR1 comprising the amino acid sequence of SEQ ID NO:99; a CDR2 comprising the amino acid sequence of SEQ ID NO:100; and a CDR3 comprising the amino acid sequence of SEQ ID NO:101; and VH4 comprises a CDR1 comprising the amino acid sequence of SEQ ID NO:96; a CDR2 comprising the amino acid sequence of SEQ ID NO:97; and a CDR3 comprising the amino acid sequence of SEQ ID NO:98. In some aspects, VL4 comprises an amino acid sequence having at least 80%, at least 85%, at least 90%, at least 95%, or 100% identity to any one of SEQ ID NOs:39, 47, 103, 422, 445, 455, 465, 475, 519, 662, 698, 734, 762, 798, 834, 854 and 862; and VH4 comprises an amino acid sequence having at least 80%, at least 85%, at least 90%, at least 95%, or 100% identity to any one of SEQ ID NOs:38, 43, 102, 423, 432, 523, 666, 702, 738, 766, 802, 838, 850 and 858. In some aspects, VL4 comprises an amino acid sequence having at least 80% identity (such as at least 85%, 90%, 91%, 92%, 93%, 94%, 95%, 96%, 97%, 98%, 99% or 100% identity) to SEQ ID NO:39; and/or VH4 comprises an amino acid sequence having at least 80% identity (such as at least 85%, 90%, 91%, 92%, 93%, 94%, 95%, 96%, 97%, 98%, 99% or 100% identity) to SEQ ID NO:38. In some aspects, VL4 comprises an amino acid sequence having at least 80% identity (such as at least 85%, 90%, 91%, 92%, 93%, 94%, 95%, 96%, 97%, 98%, 99% or 100% identity) to SEQ ID NO:47; and/or VH4 comprises an amino acid sequence having at least 80% identity (such as at least 85%, 90%, 91%, 92%, 93%, 94%, 95%, 96%, 97%, 98%, 99% or 100% identity) to SEQ ID NO:43. n some aspects, VL4 comprises an amino acid sequence having at least 80% identity (such as at least 85%, 90%, 91%, 92%, 93%, 94%, 95%, 96%, 97%, 98%, 99% or 100% identity) to SEQ ID NO:39; and/or VH4 comprises an amino acid sequence having at least 80% identity (such as at least 85%, 90%, 91%, 92%, 93%, 94%, 95%, 96%, 97%, 98%, 99% or 100% identity) to SEQ ID NO:43. In some aspects, VL4 comprises an amino acid sequence having at least 80% identity (such as at least 85%, 90%, 91%, 92%, 93%, 94%, 95%, 96%, 97%, 98%, 99% or 100% identity) to SEQ ID NO:47; and/or VH4 comprises an amino acid sequence having at least 80% identity (such as at least 85%, 90%, 91%, 92%, 93%, 94%, 95%, 96%, 97%, 98%, 99% or 100% identity) to SEQ ID NO:38. In some aspects, VL4 comprises an amino acid sequence having at least 80% identity (such as at least 85%, 90%, 91%, 92%, 93%, 94%, 95%, 96%, 97%, 98%, 99% or 100% identity) to SEQ ID NO:422; and/or VH4 comprises an amino acid sequence having at least 80% identity (such as at least 85%, 90%, 91%, 92%, 93%, 94%, 95%, 96%, 97%, 98%, 99% or 100% identity) to SEQ ID NO:423. In some aspects, VL4 comprises an amino acid sequence having at least 80% identity (such as at least 85%, 90%, 91%, 92%, 93%, 94%, 95%, 96%, 97%, 98%, 99% or 100% identity) to SEQ ID NO:445; and/or VH4 comprises an amino acid sequence having at least 80% identity (such as at least 85%, 90%, 91%, 92%, 93%, 94%, 95%, 96%, 97%, 98%, 99% or 100% identity) to SEQ ID NO:432. In some aspects, VL4 comprises an amino acid sequence having at least 80% identity (such as at least 85%, 90%, 91%, 92%, 93%, 94%, 95%, 96%, 97%, 98%, 99% or 100% identity) to SEQ ID NO:455; and/or VH4 comprises an amino acid sequence having at least 80% identity (such as at least 85%, 90%, 91%, 92%, 93%. 94%. 95%. 96%, 97%. 98%, 99% or 100% identity) to SEQ ID NO:432. In some aspects, VL4 comprises an amino acid sequence having at least 80% identity (such as at least 85%, 90%, 91%, 92%, 93%, 94%, 95%, 96%, 97%, 98%, 99% or 100% identity) to SEQ ID NO:465; and/or VH4 comprises an amino acid sequence having at least 80% identity (such as at least 85%, 90%, 91%, 92%, 93%, 94%, 95%, 96%, 97%, 98%, 99% or 100% identity) to SEQ ID NO:432. In some aspects, VL4 comprises an amino acid sequence having at least 80% identity (such as at least 85%, 90%, 91%, 92%, 93%, 94%, 95%, 96%, 97%, 98%, 99% or 100% identity) to SEQ ID NO:475; and/or VH4 comprises an amino acid sequence having at least 80% identity (such as at least 85%, 90%, 91%, 92%, 93%, 94%, 95%, 96%, 97%, 98%, 99% or 100% identity) to SEQ ID NO:432. In some aspects, VL4 comprises an amino acid sequence having at least 80% identity (such as at least 85%, 90%, 91%, 92%, 93%, 94%, 95%, 96%, 97%, 98%, 99% or 100% identity) to SEQ ID NO:519; and/or VH4 comprises an amino acid sequence having at least 80% identity (such as at least 85%, 90%, 91%, 92%, 93%, 94%, 95%, 96%, 97%, 98%, 99% or 100% identity) to SEQ ID NO:523. In some aspects, VL4 comprises an amino acid sequence having at least 80% identity (such as at least 85%, 90%, 91%, 92%, 93%, 94%, 95%, 96%, 97%, 98%, 99% or 100% identity) to SEQ ID NO:662; and/or VH4 comprises an amino acid sequence having at least 80% identity (such as at least 85%, 90%, 91%, 92%, 93%, 94%, 95%, 96%, 97%, 98%, 99% or 100% identity) to SEQ ID NO:666. In some aspects, VL4 comprises an amino acid sequence having at least 80% identity (such as at least 85%, 90%, 91%, 92%, 93%, 94%, 95%, 96%, 97%, 98%, 99% or 100% identity) to SEQ ID NO:698; and/or VH4 comprises an amino acid sequence having at least 80% identity (such as at least 85%, 90%, 91%, 92%, 93%, 94%, 95%, 96%, 97%, 98%, 99% or 100% identity) to SEQ ID NO:702. In some aspects, VL4 comprises an amino acid sequence having at least 80% identity (such as at least 85%, 90%, 91%, 92%, 93%, 94%, 95%, 96%, 97%, 98%, 99% or 100% identity) to SEQ ID NO:734; and/or VH4 comprises an amino acid sequence having at least 80% identity (such as at least 85%, 90%, 91%, 92%, 93%, 94%, 95%, 96%, 97%, 98%, 99% or 100% identity) to SEQ ID NO:738. In some aspects, VL4 comprises an amino acid sequence having at least 80% identity (such as at least 85%, 90%, 91%, 92%, 93%, 94%, 95%, 96%, 97%, 98%, 99% or 100% identity) to SEQ ID NO:762; and/or VH4 comprises an amino acid sequence having at least 80% identity (such as at least 85%, 90%, 91%, 92%, 93%, 94%, 95%, 96%, 97%, 98%, 99% or 100% identity) to SEQ ID NO:766. In some aspects, VL4 comprises an amino acid sequence having at least 80% identity (such as at least 85%, 90%, 91%, 92%, 93%, 94%, 95%, 96%, 97%, 98%, 99% or 100% identity) to SEQ ID NO:798; and/or VH4 comprises an amino acid sequence having at least 80% identity (such as at least 85%, 90%, 91%, 92%, 93%, 94%, 95%, 96%, 97%, 98%, 99% or 100% identity) to SEQ ID NO:802. In some aspects, VL4 comprises an amino acid sequence having at least 80% identity (such as at least 85%, 90%, 91%, 92%, 93%, 94%, 95%, 96%, 97%, 98%, 99% or 100% identity) to SEQ ID NO:834; and/or VH4 comprises an amino acid sequence having at least 80% identity (such as at least 85%, 90%, 91%, 92%, 93%, 94%, 95%, 96%, 97%, 98%, 99% or 100% identity) to SEQ ID NO:838. In some aspects, VL4 comprises an amino acid sequence having at least 80% identity (such as at least 85%, 90%, 91%, 92%, 93%, 94%, 95%, 96%, 97%, 98%, 99% or 100% identity) to SEQ ID NO:854; and/or VH4 comprises an amino acid sequence having at least 80% identity (such as at least 85%, 90%, 91%, 92%, 93%, 94%, 95%, 96%, 97%, 98%, 99% or 100% identity) to SEQ ID NO:850. In some aspects, VL4 comprises an amino acid sequence having at least 80% identity (such as at least 85%, 90%, 91%, 92%, 93%, 94%, 95%, 96%, 97%, 98%, 99% or 100% identity) to SEQ ID NO:862; and/or VH4 comprises an amino acid sequence having at least 80% identity (such as at least 85%, 90%, 91%, 92%, 93%, 94%, 95%, 96%, 97%, 98%, 99% or 100% identity) to SEQ ID NO:858.

In some aspects, VL2 and VH2 specifically bind to CD3. In some aspects, VL2 comprises a CDR1 comprising an amino acid sequence having at least 90% identity, at least 95% identity, or 100% identity to any one of SEQ ID NOs:35, 44, 99, 446, 456, 466, 476, 520, 663, 699, 735, 763, 799, 835, 855 and 863; a CDR2 comprising an amino acid sequence having at least 90% identity, at least 95% identity, or 100% identity to any one of SEQ ID NOs:36, 45, 100, 447 (DT), 457 (DT), 467 (DT), 477 (DT), 521, 664, 700, 736, 764, 800, 836, 856 and 864; and a CDR3 comprising an amino acid sequence having at least 90% identity, at least 95% identity, or 100% identity to any one of SEQ ID NOs:37, 46, 101, 448, 458, 468, 478, 522, 665, 701, 737, 765, 801, 837, 857 and 865; and VH2 comprises a CDR1 comprising an amino acid sequence having at least 90% identity, at least 95% identity, or 100% identity to any one of SEQ ID NOs:32, 40, 96, 433, 524, 667, 703, 739, 767, 803. 839, 851 and 859; a CDR2 comprising an amino acid sequence having at least 90% identity, at least 95% identity, or 100% identity to any one of SEQ ID NOs:33, 41, 97, 434, 525, 668, 704, 740, 768, 804, 840, 852 and 860; and a CDR3 comprising an amino acid sequence having at least 90% identity, at least 95% identity, or 100% identity to any one of SEQ ID NOs:34, 42, 98, 435, 526, 669, 705, 741, 769, 805, 841, 853 and 861. In some aspects, VL2 comprises a CDR1 comprising an amino acid sequence having at least 90% identity to SEQ ID NO:35; a CDR2 comprising an amino acid sequence having at least 90% identity to SEQ ID NO:36; and a CDR3 comprising an amino acid sequence having at least 90% identity to SEQ ID NO:37; and VH2 comprises a CDR1 comprising an amino acid sequence having at least 90% identity to SEQ ID NO:32; a CDR2 comprising an amino acid sequence having at least 90% identity to SEQ ID NO:33; and a CDR3 comprising an amino acid sequence having at least 90% identity to SEQ ID NO:34. In some aspects, VL2 comprises a CDR1 comprising an amino acid sequence having at least 90% identity to SEQ ID NO:44; a CDR2 comprising an amino acid sequence having at least 90% identity to SEQ ID NO:45; and a CDR3 comprising an amino acid sequence having at least 90% identity to SEQ ID NO:46; and VH2 comprises a CDR1 comprising an amino acid sequence having at least 90% identity to SEQ ID NO:40; a CDR2 comprising an amino acid sequence having at least 90% identity to SEQ ID NO:41; and a CDR3 comprising an amino acid sequence having at least 90% identity to SEQ ID NO:42. In some aspects, VL2 comprises a CDR1 comprising an amino acid sequence having at least 90% identity to SEQ ID NO:446; a CDR2 comprising an amino acid sequence having at least 90% identity to SEQ ID NO:447 (DT); and a CDR3 comprising an amino acid sequence having at least 90% identity to SEQ ID NO:448; and VH2 comprises a CDR1 comprising an amino acid sequence having at least 90% identity to SEQ ID NO:433; a CDR2 comprising an amino acid sequence having at least 90% identity to SEQ ID NO:434; and a CDR3 comprising an amino acid sequence having at least 90% identity to SEQ ID NO:435. In some aspects, VL2 comprises a CDR1 comprising an amino acid sequence having at least 90% identity to SEQ ID NO:456; a CDR2 comprising an amino acid sequence having at least 90% identity to SEQ ID NO:457 (DT); and a CDR3 comprising an amino acid sequence having at least 90% identity to SEQ ID NO:458; and VH2 comprises a CDR1 comprising an amino acid sequence having at least 90% identity to SEQ ID NO:433; a CDR2 comprising an amino acid sequence having at least 90% identity to SEQ ID NO:434; and a CDR3 comprising an amino acid sequence having at least 90% identity to SEQ ID NO:435. In some aspects, VL2 comprises a CDR1 comprising an amino acid sequence having at least 90% identity to SEQ ID NO:466; a CDR2 comprising an amino acid sequence having at least 90% identity to SEQ ID NO:467 (DT); and a CDR3 comprising an amino acid sequence having at least 90% identity to SEQ ID NO:468; and VH2 comprises a CDR1 comprising an amino acid sequence having at least 90% identity to SEQ ID NO:433; a CDR2 comprising an amino acid sequence having at least 90% identity to SEQ ID NO:434; and a CDR3 comprising an amino acid sequence having at least 90% identity to SEQ ID NO:435. In some aspects, VL2 comprises a CDR1 comprising an amino acid sequence having at least 90% identity to SEQ ID NO:476; a CDR2 comprising an amino acid sequence having at least 90% identity to SEQ ID NO:477 (DT); and a CDR3 comprising an amino acid sequence having at least 90% identity to SEQ ID NO:478; and VH2 comprises a CDR1 comprising an amino acid sequence having at least 90% identity to SEQ ID NO:433; a CDR2 comprising an amino acid sequence having at least 90% identity to SEQ ID NO:434; and a CDR3 comprising an amino acid sequence having at least 90% identity to SEQ ID NO:435. In some aspects, VL2 comprises a CDR1 comprising an amino acid sequence having at least 90% identity to SEQ ID NO:520; a CDR2 comprising an amino acid sequence having at least 90% identity to SEQ ID NO:521; and a CDR3 comprising an amino acid sequence having at least 90% identity to SEQ ID NO:522; and VH2 comprises a CDR1 comprising an amino acid sequence having at least 90% identity to SEQ ID NO:524; a CDR2 comprising an amino acid sequence having at least 90% identity to SEQ ID NO:525; and a CDR3 comprising an amino acid sequence having at least 90% identity to SEQ ID NO:526. In some aspects, VL2 comprises a CDR1 comprising an amino acid sequence having at least 90% identity to SEQ ID NO:663; a CDR2 comprising an amino acid sequence having at least 90% identity to SEQ ID NO:664; and a CDR3 comprising an amino acid sequence having at least 90% identity to SEQ ID NO:665; and VH2 comprises a CDR1 comprising an amino acid sequence having at least 90% identity to SEQ ID NO:667; a CDR2 comprising an amino acid sequence having at least 90% identity to SEQ ID NO:668; and a CDR3 comprising an amino acid sequence having at least 90% identity to SEQ ID NO:669. In some aspects, VL2 comprises a CDR1 comprising an amino acid sequence having at least 90% identity to SEQ ID NO:699; a CDR2 comprising an amino acid sequence having at least 90% identity to SEQ ID NO: 700; and a CDR3 comprising an amino acid sequence having at least 90% identity to SEQ ID NO:701; and VH2 comprises a CDR1 comprising an amino acid sequence having at least 90% identity to SEQ ID NO:703; a CDR2 comprising an amino acid sequence having at least 90% identity to SEQ ID NO:704; and a CDR3 comprising an amino acid sequence having at least 90% identity to SEQ ID NO:705. In some aspects, VL2 comprises a CDR1 comprising an amino acid sequence having at least 90% identity to SEQ ID NO:735; a CDR2 comprising an amino acid sequence having at least 90% identity to SEQ ID NO:736; and a CDR3 comprising an amino acid sequence having at least 90% identity to SEQ ID NO:737 and VH2 comprises a CDR1 comprising an amino acid sequence having at least 90% identity to SEQ ID NO:739; a CDR2 comprising an amino acid sequence having at least 90% identity to SEQ ID NO:740; and a CDR3 comprising an amino acid sequence having at least 90% identity to SEQ ID NO:741. In some aspects, VL2 comprises a CDR1 comprising an amino acid sequence having at least 90% identity to SEQ ID NO:763; a CDR2 comprising an amino acid sequence having at least 90% identity to SEQ ID NO:764; and a CDR3 comprising an amino acid sequence having at least 90% identity to SEQ ID NO:765; and VH2 comprises a CDR1 comprising an amino acid sequence having at least 90% identity to SEQ ID NO:767; a CDR2 comprising an amino acid sequence having at least 90% identity to SEQ ID NO:768; and a CDR3 comprising an amino acid sequence having at least 90% identity to SEQ ID NO:769. In some aspects, VL2 comprises a CDR1 comprising an amino acid sequence having at least 90% identity to SEQ ID NO:799; a CDR2 comprising an amino acid sequence having at least 90% identity to SEQ ID NO:800; and a CDR3 comprising an amino acid sequence having at least 90% identity to SEQ ID NO:801; and VH2 comprises a CDR1 comprising an amino acid sequence having at least 90% identity to SEQ ID NO:803; a CDR2 comprising an amino acid sequence having at least 90% identity to SEQ ID NO: 804; and a CDR3 comprising an amino acid sequence having at least 90% identity to SEQ ID NO:805. In some aspects, VL2 comprises a CDR1 comprising an amino acid sequence having at least 90% identity to SEQ ID NO:835; a CDR2 comprising an amino acid sequence having at least 90% identity to SEQ ID NO:836 and a CDR3 comprising an amino acid sequence having at least 90% identity to SEQ ID NO:837; and VH2 comprises a CDR1 comprising an amino acid sequence having at least 90% identity to SEQ ID NO:839; a CDR2 comprising an amino acid sequence having at least 90% identity to SEQ ID NO:840; and a CDR3 comprising an amino acid sequence having at least 90% identity to SEQ ID NO:841. In some aspects, VL2 comprises a CDR1 comprising an amino acid sequence having at least 90% identity to SEQ ID NO:855; a CDR2 comprising an amino acid sequence having at least 90% identity to SEQ ID NO:856; and a CDR3 comprising an amino acid sequence having at least 90% identity to SEQ ID NO:857; and VH2 comprises a CDR1 comprising an amino acid sequence having at least 90% identity to SEQ ID NO:851; a CDR2 comprising an amino acid sequence having at least 90% identity to SEQ ID NO:852; and a CDR3 comprising an amino acid sequence having at least 90% identity to SEQ ID NO:853. In some aspects, VL2 comprises a CDR1 comprising an amino acid sequence having at least 90% identity to SEQ ID NO:863; a CDR2 comprising an amino acid sequence having at least 90% identity to SEQ ID NO: 864; and a CDR3 comprising an amino acid sequence having at least 90% identity to SEQ ID NO:865; and VH2 comprises a CDR1 comprising an amino acid sequence having at least 90% identity to SEQ ID NO:859 a CDR2 comprising an amino acid sequence having at least 90% identity to SEQ ID NO:860; and a CDR3 comprising an amino acid sequence having at least 90% identity to SEQ ID NO:861. In some aspects, VL2 comprises a CDR1 comprising an amino acid sequence having at least 90% identity to SEQ ID NO:99; a CDR2 comprising an amino acid sequence having at least 90% identity to SEQ ID NO:100; and a CDR3 comprising an amino acid sequence having at least 90% identity to SEQ ID NO:101; and VH2 comprises a CDR1 comprising an amino acid sequence having at least 90% identity to SEQ ID NO:96; a CDR2 comprising an amino acid sequence having at least 90% identity to SEQ ID NO:97; and a CDR3 comprising an amino acid sequence having at least 90% identity to SEQ ID NO: 98. As used herein, “at least 90% identity” includes at least 91%, 92%, 93%, 94%, 95%, 96%, 97%, 98%, 99% and 100% identity to the recited reference sequence. In some aspects, VL2 comprises a CDR1 comprising the amino acid sequence of SEQ ID NO:35; a CDR2 comprising the amino acid sequence of SEQ ID NO:36; and a CDR3 comprising the amino acid sequence of SEQ ID NO:37; a″d VH2 comprises a CDR1 comprising the amino acid sequence of SEQ ID NO:32; a CDR2 comprising the amino acid sequence of SEQ ID NO:33; and a CDR3 comprising the amino acid sequence of SEQ ID NO:34. In some aspects, VL2 comprises a CDR1 comprising the amino acid sequence of SEQ ID NO:44; a CDR2 comprising the amino acid sequence of SEQ ID NO:45; and a CDR3 comprising the amino acid sequence of SEQ ID NO:46; and VH2 comprises a CDR1 comprising the amino acid sequence of SEQ ID NO:40; a CDR2 comprising the amino acid sequence of SEQ ID NO:41; and a CDR3 comprising the amino acid sequence of SEQ ID NO:42. In some aspects, VL2 comprises a CDR1 comprising the amino acid sequence of SEQ ID NO:446; a CDR2 comprising the amino acid sequence of SEQ ID NO:447 (DT); and a CDR3 comprising the amino acid sequence of SEQ ID NO:448; and VH2 comprises a CDR1 comprising the amino acid sequence of SEQ ID NO:433; a CDR2 comprising the amino acid sequence of SEQ ID NO:434; and a CDR3 comprising the amino acid sequence of SEQ ID NO:435. In some aspects, VL2 comprises a CDR1 comprising the amino acid sequence of SEQ ID NO:456; a CDR2 comprising the amino acid sequence of SEQ ID NO:457 (DT); and a CDR3 comprising the amino acid sequence of SEQ ID NO:458; and VH2 comprises a CDR1 comprising the amino acid sequence of SEQ ID NO:433; a CDR2 comprising the amino acid sequence of SEQ ID NO:434; and a CDR3 comprising the amino acid sequence of SEQ ID NO:435. In some aspects, VL2 comprises a CDR1 comprising the amino acid sequence of SEQ ID NO:466; a CDR2 comprising the amino acid sequence of SEQ ID NO:467 (DT); and a CDR3 comprising the amino acid sequence of SEQ ID NO:468; and VH2 comprises a CDR1 comprising the amino acid sequence of SEQ ID NO:433; a CDR2 comprising the amino acid sequence of SEQ ID NO:434; and a CDR3 comprising the amino acid sequence of SEQ ID NO:435. In some aspects, VL2 comprises a CDR1 comprising the amino acid sequence of SEQ ID NO:476; a CDR2 comprising the amino acid sequence of SEQ ID NO:477 (DT); and a CDR3 comprising the amino acid sequence of SEQ ID NO:478; and VH2 comprises a CDR1 the amino acid sequence of SEQ ID NO:433; a CDR2 comprising the amino acid sequence of SEQ ID NO:434; and a CDR3 comprising the amino acid sequence of SEQ ID NO:435. In some aspects, VL2 comprises a CDR1 comprising the amino acid sequence of SEQ ID NO:520; a CDR2 comprising the amino acid sequence of SEQ ID NO:521; and a CDR3 comprising the amino acid sequence of SEQ ID NO:522; and VH2 comprises a CDR1 comprising the amino acid sequence of SEQ ID NO:524; a CDR2 comprising the amino acid sequence of SEQ ID NO:525; and a CDR3 comprising the amino acid sequence of SEQ ID NO:526. In some aspects, VL2 comprises a CDR1 comprising the amino acid sequence of SEQ ID NO:663; a CDR2 comprising the amino acid sequence of SEQ ID NO:664; and a CDR3 comprising the amino acid sequence of SEQ ID NO:665; and VH2 comprises a CDR1 comprising the amino acid sequence of SEQ ID NO:667; a CDR2 comprising the amino acid sequence of SEQ ID NO:668; and a CDR3 comprising the amino acid sequence of SEQ ID NO:669. In some aspects, VL2 comprises a CDR1 comprising the amino acid sequence of SEQ ID NO:699; a CDR2 comprising the amino acid sequence of SEQ ID NO: 700; and a CDR3 comprising the amino acid sequence of SEQ ID NO:701; and VH2 comprises a CDR1 comprising the amino acid sequence of SEQ ID NO:703; a CDR2 comprising the amino acid sequence of SEQ ID NO:704; and a CDR3 comprising the amino acid sequence of SEQ ID NO:705. In some aspects, VL2 comprises a CDR1 comprising the amino acid sequence of SEQ ID NO:735; a CDR2 comprising the amino acid sequence of SEQ ID NO:736; and a CDR3 comprising the amino acid sequence of SEQ ID NO:737 and VH2 comprises a CDR1 comprising the amino acid sequence of SEQ ID NO:739; a CDR2 comprising the amino acid sequence of SEQ ID NO:740; and a CDR3 comprising the amino acid sequence of SEQ ID NO:741. In some aspects, VL2 comprises a CDR1 comprising the amino acid sequence of SEQ ID NO:763; a CDR2 comprising the amino acid sequence of SEQ ID NO:764; and a CDR3 comprising the amino acid sequence of SEQ ID NO:765; and VH2 comprises a CDR1 comprising the amino acid sequence of SEQ ID NO:767; a CDR2 the amino acid sequence of SEQ ID NO:768; and a CDR3 comprising the amino acid sequence of SEQ ID NO:769. In some aspects, VL2 comprises a CDR1 comprising the amino acid sequence of SEQ ID NO:799; a CDR2 comprising the amino acid sequence of SEQ ID NO:800; and a CDR3 comprising the amino acid sequence of SEQ ID NO:801; and VH2 comprises a CDR1 comprising an amino acid sequence having at least 90% identity to SEQ ID NO:803; a CDR2 comprising the amino acid sequence of SEQ ID NO:804; and a CDR3 comprising the amino acid sequence of SEQ ID NO:805. In some aspects, VL2 comprises a CDR1 comprising the amino acid sequence of SEQ ID NO:835; a CDR2 comprising the amino acid sequence of SEQ ID NO:836 and a CDR3 comprising the amino acid sequence of SEQ ID NO:837; and VH2 comprises a CDR1 comprising the amino acid sequence of SEQ ID NO:839; a CDR2 comprising the amino acid sequence of SEQ ID NO:840; and a CDR3 comprising the amino acid sequence of SEQ ID NO:841. In some aspects, VL2 comprises a CDR1 comprising the amino acid sequence of SEQ ID NO:855; a CDR2 comprising the amino acid sequence of SEQ ID NO:856; and a CDR3 comprising the amino acid sequence of SEQ ID NO:857; and VH2 comprises a CDR1 comprising the amino acid sequence of SEQ ID NO:851; a CDR2 comprising the amino acid sequence of SEQ ID NO:852; and a CDR3 comprising the amino acid sequence of SEQ ID NO:853. In some aspects, VL2 comprises a CDR1 comprising the amino acid sequence of SEQ ID NO:863; a CDR2 comprising the amino acid sequence of SEQ ID NO: 864; and a CDR3 comprising the amino acid sequence of SEQ ID NO:865; and VH2 comprises a CDR1 comprising the amino acid sequence of SEQ ID NO:859 a CDR2 comprising the amino acid sequence of SEQ ID NO:860; and a CDR3 comprising the amino acid sequence of SEQ ID NO:861. In some aspects, VL2 comprises a CDR1 comprising the amino acid sequence of SEQ ID NO:99; a CDR2 comprising the amino acid sequence of SEQ ID NO:100; and a CDR3 comprising the amino acid sequence of SEQ ID NO:101; and VH2 comprises a CDR1 comprising the amino acid sequence of SEQ ID NO:96; a CDR2 comprising the amino acid sequence of SEQ ID NO:97; and a CDR3 comprising the amino acid sequence of SEQ ID NO:98. In some aspects, VL2 comprises an amino acid sequence having at least 80%, at least 85%, at least 90%, at least 95%, or 100% identity to any one of SEQ ID NOs:39, 47, 103, 422, 445, 455, 465, 475, 519, 662, 698, 734, 762, 798, 834, 854 and 862; and VH2 comprises an amino acid sequence having at least 80%, at least 85%, at least 90%, at least 95%, or 100% identity to any one of SEQ ID NOs:38, 43, 102. 423, 432, 523, 666, 702, 738, 766, 802, 838, 850 and 858. In some aspects, VL2 comprises an amino acid sequence having at least 80% identity (such as at least 85%, 90%, 91%, 92%, 93%, 94%, 95%, 96%, 97%, 98%, 99% or 100% identity) to SEQ ID NO:39; and/or VH2 comprises an amino acid sequence having at least 80% identity (such as at least 85%, 90%, 91%, 92%, 93%, 94%, 95%, 96%, 97%, 98%, 99% or 100% identity) to SEQ ID NO:38. In some aspects, VL2 comprises an amino acid sequence having at least 80% identity (such as at least 85%, 90%, 91%, 92%, 93%, 94%, 95%, 96%, 97%, 98%, 99% or 100% identity) to SEQ ID NO:47; and/or VH2 comprises an amino acid sequence having at least 80% identity (such as at least 85%, 90%, 91%, 92%, 93%, 94%, 95%, 96%, 97%. 98%, 99% or 100% identity) to SEQ ID NO:43. In some aspects, VL2 comprises an amino acid sequence having at least 80% identity (such as at least 85%, 90%, 91%, 92%, 93%, 94%, 95%, 96%, 97%, 98%, 99% or 100% identity) to SEQ ID NO:39; and/or VH2 comprises an amino acid sequence having at least 80% identity (such as at least 85%, 90%, 91%, 92%, 93%, 94%, 95%, 96%, 97%, 98%, 99% or 100% identity) to SEQ ID NO:43. In some aspects, VL2 comprises an amino acid sequence having at least 80% identity (such as at least 85%, 90%, 91%, 92%, 93%, 94%, 95%, 96%, 97%, 98%, 99% or 100% identity) to SEQ ID NO:47; and/or VH2 comprises an amino acid sequence having at least 80% identity (such as at least 85%, 90%, 91%, 92%, 93%, 94%, 95%, 96%, 97%, 98%, 99% or 100% identity) to SEQ ID NO:38. In some aspects, VL2 comprises an amino acid sequence having at least 80% identity (such as at least 85%, 90%, 91%, 92%, 93%, 94%, 95%, 96%, 97%, 98%, 99% or 100% identity) to SEQ ID NO:422; and/or VH2 comprises an amino acid sequence having at least 80% identity (such as at least 85%, 90%, 91%, 92%, 93%, 94%, 95%, 96%, 97%, 98%, 99% or 100% identity) to SEQ ID NO:423. In some aspects, VL2 comprises an amino acid sequence having at least 80% identity (such as at least 85%, 90%, 91%, 92%, 93%, 94%, 95%, 96%, 97%, 98%, 99% or 100% identity) to SEQ ID NO:445; and/or VH2 comprises an amino acid sequence having at least 80% identity (such as at least 85%, 90%, 91%, 92%, 93%, 94%, 95%, 96%, 97%, 98%, 99% or 100% identity) to SEQ ID NO:432. In some aspects, VL2 comprises an amino acid sequence having at least 80% identity (such as at least 85%, 90%, 91%, 92%, 93%, 94%, 95%, 96%, 97%, 98%, 99% or 100% identity) to SEQ ID NO:455; and/or VH2 comprises an amino acid sequence having at least 80% identity (such as at least 85%, 90%, 91%, 92%, 93%, 94%, 95%, 96%, 97%, 98%, 99% or 100% identity) to SEQ ID NO:432. In some aspects, VL2 comprises an amino acid sequence having at least 80% identity (such as at least 85%, 90%, 91%, 92%, 93%, 94%, 95%, 96%, 97%, 98%, 99% or 100% identity) to SEQ ID NO:465; and/or VH2 comprises an amino acid sequence having at least 80% identity (such as at least 85%, 90%, 91%, 92%, 93%, 94%, 95%, 96%, 97%, 98%, 99% or 100% identity) to SEQ ID NO:432. In some aspects, VL4 comprises an amino acid sequence having at least 80% identity (such as at least 85%, 90%, 91%, 92%, 93%, 94%, 95%, 96%, 97%, 98%, 99% or 100% identity) to SEQ ID NO:475; and/or VH2 comprises an amino acid sequence having at least 80% identity (such as at least 85%, 90%, 91%, 92%, 93%, 94%, 95%, 96%, 97%, 98%, 99% or 100% identity) to SEQ ID NO:432. In some aspects, VL2 comprises an amino acid sequence having at least 80% identity (such as at least 85%, 90%, 91%, 92%, 93%, 94%, 95%, 96%, 97%, 98%, 99% or 100% identity) to SEQ ID NO:519; and/or VH2 comprises an amino acid sequence having at least 80% identity (such as at least 85%, 90%, 91%, 92%, 93%, 94%, 95%, 96%, 97%, 98%, 99% or 100% identity) to SEQ ID NO:523. In some aspects, VL2 comprises an amino acid sequence having at least 80% identity (such as at least 85%, 90%, 91%, 92%, 93%, 94%, 95%, 96%, 97%, 98%, 99% or 100% identity) to SEQ ID NO:662; and/or VH2 comprises an amino acid sequence having at least 80% identity (such as at least 85%, 90%, 91%, 92%, 93%, 94%, 95%, 96%, 97%, 98%, 99% or 100% identity) to SEQ ID NO:666. In some aspects, VL2 comprises an amino acid sequence having at least 80% identity (such as at least 85%, 90%, 91%, 92%, 93%, 94%, 95%, 96%, 97%, 98%, 99% or 100% identity) to SEQ ID NO:698; and/or VH2 comprises an amino acid sequence having at least 80% identity (such as at least 85%, 90%, 91%, 92%, 93%, 94%, 95%, 96%, 97%, 98%, 99% or 100% identity) to SEQ ID NO:702. In some aspects, VL2 comprises an amino acid sequence having at least 80% identity (such as at least 85%, 90%, 91%, 92%, 93%, 94%, 95%, 96%, 97%, 98%, 99% or 100% identity) to SEQ ID NO:734; and/or VH2 comprises an amino acid sequence having at least 80% identity (such as at least 85%, 90%, 91%, 92%, 93%, 94%, 95%, 96%, 97%, 98%, 99% or 100% identity) to SEQ ID NO:738. In some aspects, VL2 comprises an amino acid sequence having at least 80% identity (such as at least 85%, 90%, 91%, 92%, 93%, 94%, 95%, 96%, 97%, 98%, 99% or 100% identity) to SEQ ID NO:762; and/or VH2 comprises an amino acid sequence having at least 80% identity (such as at least 85%, 90%, 91%, 92%, 93%, 94%, 95%, 96%, 97%, 98%, 99% or 100% identity) to SEQ ID NO:766. In some aspects, VL2 comprises an amino acid sequence having at least 80% identity (such as at least 85%, 90%, 91%, 92%, 93%, 94%, 95%, 96%, 97%, 98%, 99% or 100% identity) to SEQ ID NO:798; and/or VH2 comprises an amino acid sequence having at least 80% identity (such as at least 85%, 90%, 91%, 92%, 93%, 94%, 95%, 96%, 97%, 98%, 99% or 100% identity) to SEQ ID NO:802. In some aspects, VL2 comprises an amino acid sequence having at least 80% identity (such as at least 85%, 90%, 91%, 92%, 93%, 94%, 95%, 96%, 97%, 98%, 99% or 100% identity) to SEQ ID NO:834; and/or VH2 comprises an amino acid sequence having at least 80% identity (such as at least 85%, 90%, 91%, 92%, 93%, 94%, 95%, 96%, 97%, 98%, 99% or 100% identity) to SEQ ID NO:838. In some aspects, VL2 comprises an amino acid sequence having at least 80% identity (such as at least 85%, 90%, 91%, 92%, 93%, 94%, 95%, 96%, 97%, 98%, 99% or 100% identity) to SEQ ID NO:854; and/or VH2 comprises an amino acid sequence having at least 80% identity (such as at least 85%, 90%, 91%, 92%, 93%, 94%, 95%, 96%, 97%, 98%, 99% or 100% identity) to SEQ ID NO:850. In some aspects, VL2 comprises an amino acid sequence having at least 80% identity (such as at least 85%, 90%, 91%, 92%, 93%, 94%, 95%, 96%, 97%, 98%, 99% or 100% identity) to SEQ ID NO:862; and/or VH2 comprises an amino acid sequence having at least 80% identity (such as at least 85%, 90%, 91%, 92%, 93%, 94%, 95%, 96%, 97%, 98%, 99% or 100% identity) to SEQ ID NO:858.

In some aspects, the antigen binding polypeptide complex (e.g., antibody or antigen binding fragment thereof) of the invention specifically binds to an immune stimulating receptor. In some aspects, the antigen binding polypeptide complex specifically binds to CD3 and an immune stimulating receptor. In some aspects, the antigen binding polypeptide complex specifically binds to CD3, an immune stimulating receptor, and one or more TAAs, such as, e.g., A2AR, APRIL, ATPDase, BAFF, BAFFR, BCMA, BlyS, BTK, BTLA, B7DC, B7H1, B7H2, B7H3, B7H4, B7H5, B7H6, B7H7, B7RP1, B7-4, C3, C5, CCL2, CCL3, CCL4, CCL5, CCL7, CCL8, CCL11, CCL15, CCL17, CCL19, CCL20, CCL21, CCL25, CCR3, CCR4, CD16A, CD19, CD20, CD24, CD27, CD28, CD30, CD38, CD39, CD40, CD40L, CD47, CD52, CD70, CD80, CD86, CD123, CD133, CD137, CD137L, CD160, CD272, CEACAM5, CLEC9, CLEC91, CRTH2, CSF-1, CSF-2, CSF-3, CXCL1, CXCL2, CXCL4, CXCL12, CXCL13, CXCR3, cMet, CTLA4, DLL3, DLL4, DNGR-1, E-cadherin, EGFR, ENTPD1, EpCAM, FCER1, FCER1A, FCER2, FGFR, FLAP, FOLH1, Gi24, GITR, GITRL, GPR5, GP100, GPRC5D, HER2, HER3, ICOSL, ICOS, HHLA2, HMGB1, HVEM, IDO, IFNa, IgE, IGF1R, IL2Rbeta, IL1, ILIA, IL1B, IL1F10, IL2, IL4, IL4Ra, IL5, IL5R, IL6, IL7, IL7Ra, IL8, IL9, IL9R, IL10, rhILlO, IL12, IL13, IL13Ra1, IL13Ra2, IL15, IL17, IL17Rb, IL18, IL22, IL23, IL25, IL7, IL33, IL35, ITGB4, ITK, KIR, LAG3, LAMP1, leptin, LPFS2, MHC class II, MUC-1, MUC-16, NCR3LG1, NKG2D, NKp46, NTPDase-1, OX40, OX40L, PD-1, PD-L1, PD-L2, PROM1, S152, SIRPalpha, SISP1, SLC, SPG64, ST2, STEAP1, STEAP2, Syk kinase, STEAP1, TROP2, TACI, TDO, TGFBETA, T14, TIGIT, TIM3, TLR, TLR2, TLR4, TLR5, TLR9, TMEF1, TNFa, TNFRSF7, Tp55, TREM1, TSLP, TSLPR, TWEAK, VEGF, VISTA, Vstm3, or WUCAM. For example, the antigen binding polypeptide complex may specifically bind CD3, an immune stimulating receptor (such as CD28), CD19 and CD20. For example, the antigen binding polypeptide complex may specifically bind CD3, an immune stimulating receptor (such as CD28), cMet and Trop2.

In some aspects, a VL and a corresponding VH of the antigen binding polypeptide complex specifically bind to an immune stimulating receptor (such as CD28). In some aspects, one VL and one corresponding VH of the antigen binding polypeptide complex specifically bind to an immune stimulating receptor. In some aspects, VL1 and VH1 specifically bind to an immune stimulating receptor. In some aspects, VL1 comprises a CDR1 comprising an amino acid sequence having at least 90% identity, at least 95% identity, or 100% identity to any one of SEQ ID NOs:51, 59, 645, 681, 771, 843, 717 and 807; a CDR2 comprising an amino acid sequence having at least 90% identity, at least 95% identity, or 100% identity to any one of SEQ ID NOs:52, 60, 646, 682, 772, 844, 718 and 808; and a CDR3 comprising an amino acid sequence having at least 90% identity, at least 95% identity, or 100% identity to any one of SEQ ID NOs:53, 61, 647, 683, 773, 845, 719 and 809; and VH1 comprises a CDR1 comprising an amino acid sequence having at least 90% identity, at least 95% identity, or 100% identity to any one of SEQ ID NOs:48, 56, 649, 685, 775, 847, 721 and 811; a CDR2 comprising an amino acid sequence having at least 90% identity, at least 95% identity, or 100% identity to any one of SEQ ID NOs:49, 57, 650, 686, 776, 848, 722 and 812; and a CDR3 comprising an amino acid sequence having at least 90% identity, at least 95% identity, or 100% identity to any one of SEQ ID NOs:50, 58, 651, 687, 777, 849, 723 and 813. In some aspects, VL1 comprises a CDR1 comprising an amino acid sequence having at least 90% identity to SEQ ID NO:51; a CDR2 comprising an amino acid sequence having at least 90% identity to SEQ ID NO:52; and a CDR3 comprising an amino acid sequence having at least 90% identity to SEQ ID NO:53; and VH1 comprises a CDR1 comprising an amino acid sequence having at least 90% identity to SEQ ID NO:48; a CDR2 comprising an amino acid sequence having at least 90% identity to SEQ ID NO:49; and a CDR3 comprising an amino acid sequence having at least 90% identity to SEQ ID NO:50. In some aspects, VL1 comprises a CDR1 comprising an amino acid sequence having at least 90% identity to SEQ ID NO:59; a CDR2 comprising an amino acid sequence having at least 90% identity to SEQ ID NO:60; and a CDR3 comprising an amino acid sequence having at least 90% identity to SEQ ID NO:61; and VH1 comprises a CDR1 comprising an amino acid sequence having at least 90% identity to SEQ ID NO:56; a CDR2 comprising an amino acid sequence having at least 90% identity to SEQ ID NO:57; and a CDR3 comprising an amino acid sequence having at least 90% identity to SEQ ID NO:58. In some aspects, VL1 comprises a CDR1 comprising an amino acid sequence having at least 90% identity to SEQ ID NO:645; a CDR2 comprising an amino acid sequence having at least 90% identity to SEQ ID NO:646; and a CDR3 comprising an amino acid sequence having at least 90% identity to SEQ ID NO:647; and VH1 comprises a CDR1 comprising an amino acid sequence having at least 90% identity to SEQ ID NO:649; a CDR2 comprising an amino acid sequence having at least 90% identity to SEQ ID NO:650; and a CDR3 comprising an amino acid sequence having at least 90% identity to SEQ ID NO:651. In some aspects, VL1 comprises a CDR1 comprising an amino acid sequence having at least 90% identity to SEQ ID NO:681; a CDR2 comprising an amino acid sequence having at least 90% identity to SEQ ID NO:682; and a CDR3 comprising an amino acid sequence having at least 90% identity to SEQ ID NO:683; and VH1 comprises a CDR1 comprising an amino acid sequence having at least 90% identity to SEQ ID NO:685; a CDR2 comprising an amino acid sequence having at least 90% identity to SEQ ID NO:686; and a CDR3 comprising an amino acid sequence having at least 90% identity to SEQ ID NO:687. In some aspects, VL1 comprises a CDR1 comprising an amino acid sequence having at least 90% identity to SEQ ID NO:771; a CDR2 comprising an amino acid sequence having at least 90% identity to SEQ ID NO:772; and a CDR3 comprising an amino acid sequence having at least 90% identity to SEQ ID NO:773; and VH1 comprises a CDR1 comprising an amino acid sequence having at least 90% identity to SEQ ID NO:775; a CDR2 comprising an amino acid sequence having at least 90% identity to SEQ ID NO:776; and a CDR3 comprising an amino acid sequence having at least 90% identity to SEQ ID NO:777. In some aspects, VL1 comprises a CDR1 comprising an amino acid sequence having at least 90% identity to SEQ ID NO:843; a CDR2 comprising an amino acid sequence having at least 90% identity to SEQ ID NO:844; and a CDR3 comprising an amino acid sequence having at least 90% identity to SEQ ID NO:845; and VH1 comprises a CDR1 comprising an amino acid sequence having at least 90% identity to SEQ ID NO:847; a CDR2 comprising an amino acid sequence having at least 90% identity to SEQ ID NO: 848; and a CDR3 comprising an amino acid sequence having at least 90% identity to SEQ ID NO:849. As used herein, “at least 90% identity” includes at least 91%, 92%, 93%, 94%, 95%, 96%, 97%, 98%, 99% and 100% identity to the recited reference sequence. In some aspects, VL1 comprises a CDR1 comprising the amino acid sequence of SEQ ID NO:51; a CDR2 comprising the amino acid sequence of SEQ ID NO:52; and a CDR3 comprising the amino acid sequence of SEQ ID NO:53; and VH1 comprises a CDR1 comprising the amino acid sequence of SEQ ID NO:48; a CDR2 comprising the amino acid sequence of SEQ ID NO:49; and a CDR3 comprising the amino acid sequence of SEQ ID NO:50. In some aspects, VL1 comprises a CDR1 comprising the amino acid sequence of SEQ ID NO:59; a CDR2 comprising the amino acid sequence of SEQ ID NO:60; and a CDR3 comprising the amino acid sequence of SEQ ID NO:61; and VH1 comprises a CDR1 comprising the amino acid sequence of SEQ ID NO:56; a CDR2 comprising the amino acid sequence of SEQ ID NO:57; and a CDR3 comprising the amino acid sequence of SEQ ID NO:58. In some aspects, VL1 comprises a CDR1 comprising the amino acid sequence of SEQ ID NO:645; a CDR2 comprising the amino acid sequence of SEQ ID NO:646; and a CDR3 comprising the amino acid sequence of SEQ ID NO:647; and VH1 comprises a CDR1 comprising the amino acid sequence of SEQ ID NO:649; a CDR2 comprising the amino acid sequence of SEQ ID NO:650; and a CDR3 comprising the amino acid sequence of SEQ ID NO:651. In some aspects, VL1 comprises a CDR1 comprising the amino acid sequence of SEQ ID NO:681; a CDR2 comprising the amino acid sequence of SEQ ID NO:682; and a CDR3 comprising the amino acid sequence of SEQ ID NO:683; and VH1 comprises a CDR1 comprising the amino acid sequence of SEQ ID NO:685; a CDR2 comprising the amino acid sequence of SEQ ID NO:686; and a CDR3 comprising the amino acid sequence of SEQ ID NO:687. In some aspects, VL1 comprises a CDR1 comprising the amino acid sequence of SEQ ID NO:771; a CDR2 comprising the amino acid sequence of SEQ ID NO:772; and a CDR3 comprising the amino acid sequence of SEQ ID NO:773; and VH1 comprises a CDR1 comprising the amino acid sequence of SEQ ID NO:775; a CDR2 comprising the amino acid sequence of SEQ ID NO:776; and a CDR3 comprising the amino acid sequence of SEQ ID NO:777. In some aspects, VL1 comprises a CDR1 comprising the amino acid sequence of SEQ ID NO:843; a CDR2 comprising the amino acid sequence of SEQ ID NO:844; and a CDR3 comprising the amino acid sequence of SEQ ID NO:845; and VH1 comprises a CDR1 comprising the amino acid sequence of SEQ ID NO:847; a CDR2 comprising the amino acid sequence of SEQ ID NO:848; and a CDR3 comprising the amino acid sequence of SEQ ID NO:849. In some aspects, VL1 comprises an amino acid sequence having at least 80%, at least 85%, at least 90%, at least 95%, or 100% identity to SEQ ID NO:55 or 63; and VH1 comprises an amino acid sequence having at least 80%, at least 85%, at least 90%, at least 95%, or 100% identity to SEQ ID NO:54 or 62. In some aspects, VL1 comprises an amino acid sequence having at least 80% identity (such as at least 85%, 90%, 91%, 92%, 93%, 94%, 95%, 96%, 97%, 98%, 99% or 100% identity) to SEQ ID NO:55; and/or VH1 comprises an amino acid sequence having at least 80% identity (such as at least 85%, 90%, 91%, 92%, 93%, 94%, 95%, 96%, 97%, 98%, 99% or 100% identity) to SEQ ID NO:54. In some aspects, VL1 comprises an amino acid sequence having at least 80% identity (such as at least 85%, 90%, 91%, 92%, 93%, 94%, 95%, 96%, 97%, 98%, 99% or 100% identity) to SEQ ID NO:63; and/or VH1 comprises an amino acid sequence having at least 80% identity (such as at least 85%, 90%, 91%, 92%, 93%, 94%, 95%, 96%, 97%, 98%, 99% or 100% identity) to SEQ ID NO:62. In some aspects, VL1 comprises an amino acid sequence having at least 80% identity (such as at least 85%, 90%, 91%, 92%, 93%, 94%, 95%, 96%, 97%, 98%, 99% or 100% identity) to SEQ ID NO:55; and/or VH1 comprises an amino acid sequence having at least 80% identity (such as at least 85%, 90%, 91%, 92%, 93%, 94%, 95%, 96%, 97%, 98%, 99% or 100% identity) to SEQ ID NO:62. In some aspects, VL1 comprises an amino acid sequence having at least 80% identity (such as at least 85%, 90%, 91%, 92%, 93%. 94%. 95%. 96%, 97%. 98%, 99% or 100% identity) to SEQ ID NO:63; and/or VH1 comprises an amino acid sequence having at least 80% identity (such as at least 85%, 90%, 91%, 92%, 93%, 94%, 95%, 96%, 97%, 98%, 99% or 100% identity) to SEQ ID NO:54.

In some aspects, VL2 and VH2 specifically bind to an immune stimulating receptor (such sa CD28). In some aspects, VL2 comprises a CDR1 comprising an amino acid sequence having at least 90% identity, at least 95% identity, or 100% identity to any one of SEQ ID NOs:51, 59, 645, 681, 771 and 843; a CDR2 comprising an amino acid sequence having at least 90% identity, at least 95% identity, or 100% identity to any one of SEQ ID NOs:52, 60, 646, 682, 772 and 844; and a CDR3 comprising an amino acid sequence having at least 90% identity, at least 95% identity, or 100% identity to any one of SEQ ID NOs:53, 61, 647, 683, 773 and 845; and VH2 comprises a CDR1 comprising an amino acid sequence having at least 90% identity, at least 95% identity, or 100% identity to any one of SEQ ID NOs:48, 56, 649, 685, 775 and 847; a CDR2 comprising an amino acid sequence having at least 90% identity, at least 95% identity, or 100% identity to any one of SEQ ID NOs:49, 57, 650, 686, 776 and 848; and a CDR3 comprising an amino acid sequence having at least 90% identity, at least 95% identity, or 100% identity to any one of SEQ ID NOs:50, 58, 651, 687, 777 and 849. In some aspects, VL2 comprises a CDR1 comprising an amino acid sequence having at least 90% identity to SEQ ID NO:51; a CDR2 comprising an amino acid sequence having at least 90% identity to SEQ ID NO:52; and a CDR3 comprising an amino acid sequence having at least 90% identity to SEQ ID NO:53; and VH2 comprises a CDR1 comprising an amino acid sequence having at least 90% identity to SEQ ID NO:48; a CDR2 comprising an amino acid sequence having at least 90% identity to SEQ ID NO:49; and a CDR3 comprising an amino acid sequence having at least 90% identity to SEQ ID NO:50. In some aspects, VL2 comprises a CDR1 comprising an amino acid sequence having at least 90% identity to SEQ ID NO:59; a CDR2 comprising an amino acid sequence having at least 90% identity to SEQ ID NO:60; and a CDR3 comprising an amino acid sequence having at least 90% identity to SEQ ID NO:61; and VH2 comprises a CDR1 comprising an amino acid sequence having at least 90% identity to SEQ ID NO:56; a CDR2 comprising an amino acid sequence having at least 90% identity to SEQ ID NO:57; and a CDR3 comprising an amino acid sequence having at least 90% identity to SEQ ID NO:58. In some aspects, VL2 comprises a CDR1 comprising an amino acid sequence having at least 90% identity to SEQ ID NO:645; a CDR2 comprising an amino acid sequence having at least 90% identity to SEQ ID NO:646; and a CDR3 comprising an amino acid sequence having at least 90% identity to SEQ ID NO:647; and VH2 comprises a CDR1 comprising an amino acid sequence having at least 90% identity to SEQ ID NO:649; a CDR2 comprising an amino acid sequence having at least 90% identity to SEQ ID NO:650; and a CDR3 comprising an amino acid sequence having at least 90% identity to SEQ ID NO:651. In some aspects, VL2 comprises a CDR1 comprising an amino acid sequence having at least 90% identity to SEQ ID NO:681; a CDR2 comprising an amino acid sequence having at least 90% identity to SEQ ID NO:682; and a CDR3 comprising an amino acid sequence having at least 90% identity to SEQ ID NO:683; and VH2 comprises a CDR1 comprising an amino acid sequence having at least 90% identity to SEQ ID NO:685; a CDR2 comprising an amino acid sequence having at least 90% identity to SEQ ID NO:686; and a CDR3 comprising an amino acid sequence having at least 90% identity to SEQ ID NO:687. In some aspects, VL2 comprises a CDR1 comprising an amino acid sequence having at least 90% identity to SEQ ID NO:771; a CDR2 comprising an amino acid sequence having at least 90% identity to SEQ ID NO:772; and a CDR3 comprising an amino acid sequence having at least 90% identity to SEQ ID NO:773; and VH2 comprises a CDR1 comprising an amino acid sequence having at least 90% identity to SEQ ID NO:775; a CDR2 comprising an amino acid sequence having at least 90% identity to SEQ ID NO:776; and a CDR3 comprising an amino acid sequence having at least 90% identity to SEQ ID NO:777. In some aspects, VL2 comprises a CDR1 comprising an amino acid sequence having at least 90% identity to SEQ ID NO:843; a CDR2 comprising an amino acid sequence having at least 90% identity to SEQ ID NO: 844; and a CDR3 comprising an amino acid sequence having at least 90% identity to SEQ ID NO:845; and VH2 comprises a CDR1 comprising an amino acid sequence having at least 90% identity to SEQ ID NO:847; a CDR2 comprising an amino acid sequence having at least 90% identity to SEQ ID NO: 848; and a CDR3 comprising an amino acid sequence having at least 90% identity to SEQ ID NO:849. As used herein, “at least 90% identity” includes at least 91%, 92%, 93%, 94%, 95%, 96%, 97%, 98%, 99% and 100% identity to the recited reference sequence. In some aspects, VL2 comprises a CDR1 comprising the amino acid sequence of SEQ ID NO:51; a CDR2 comprising the amino acid sequence of SEQ ID NO:52; and a CDR3 comprising the amino acid sequence of SEQ ID NO:53; and VH2 comprises a CDR1 comprising the amino acid sequence of SEQ ID NO:48; a CDR2 comprising the amino acid sequence of SEQ ID NO:49; and a CDR3 comprising the amino acid sequence of SEQ ID NO:50. In some aspects, VL2 comprises a CDR1 comprising the amino acid sequence of SEQ ID NO:59; a CDR2 comprising the amino acid sequence of SEQ ID NO:60; and a CDR3 comprising the amino acid sequence of SEQ ID NO:61; and VH2 comprises a CDR1 comprising the amino acid sequence of SEQ ID NO:56; a CDR2 comprising the amino acid sequence of SEQ ID NO:57; and a CDR3 comprising the amino acid sequence of SEQ ID NO:58. In some aspects, VL2 comprises a CDR1 comprising the amino acid sequence of SEQ ID NO:645; a CDR2 comprising the amino acid sequence of SEQ ID NO:646; and a CDR3 comprising the amino acid sequence of SEQ ID NO:647; and VH2 comprises a CDR1 comprising the amino acid sequence of SEQ ID NO:649; a CDR2 comprising the amino acid sequence of SEQ ID NO:650; and a CDR3 comprising the amino acid sequence of SEQ ID NO:651. In some aspects, VL2 comprises a CDR1 comprising the amino acid sequence of SEQ ID NO:681; a CDR2 comprising the amino acid sequence of SEQ ID NO:682; and a CDR3 comprising the amino acid sequence of SEQ ID NO:683; and VH2 comprises a CDR1 comprising the amino acid sequence of SEQ ID NO:685; a CDR2 comprising the amino acid sequence of SEQ ID NO:686; and a CDR3 comprising the amino acid sequence of SEQ ID NO:687. In some aspects, VL2 comprises a CDR1 comprising the amino acid sequence of SEQ ID NO:771; a CDR2 comprising the amino acid sequence of SEQ ID NO:772; and a CDR3 comprising the amino acid sequence of SEQ ID NO:773; and VH2 comprises a CDR1 comprising the amino acid sequence of SEQ ID NO:775; a CDR2 comprising the amino acid sequence of SEQ ID NO:776; and a CDR3 comprising the amino acid sequence of SEQ ID NO:777. In some aspects, VL2 comprises a CDR1 comprising the amino acid sequence of SEQ ID NO:843; a CDR2 comprising the amino acid sequence of SEQ ID NO:844; and a CDR3 comprising the amino acid sequence of SEQ ID NO:845; and VH2 comprises a CDR1 comprising the amino acid sequence of SEQ ID NO:847; a CDR2 comprising the amino acid sequence of SEQ ID NO:848; and a CDR3 comprising the amino acid sequence of SEQ ID NO:849. In some aspects, VL2 comprises an amino acid sequence having at least 80%, at least 85%, at least 90%, at least 95%, or 100% identity to SEQ ID NO:55 or 63; and VH2 comprises an amino acid sequence having at least 80%, at least 85%, at least 90%, at least 95%, or 100% identity to SEQ ID NO:54 or 62. In some aspects, VL2 comprises an amino acid sequence having at least 80% identity (such as at least 85%, 90%, 91%, 92%, 93%, 94%, 95%, 96%, 97%, 98%, 99% or 100% identity) to SEQ ID NO:55; and/or VH2 comprises an amino acid sequence having at least 80% identity (such as at least 85%, 90%, 91%, 92%, 93%, 94%, 95%, 96%, 97%, 98%, 99% or 100% identity) to SEQ ID NO:54. In some aspects, VL2 comprises an amino acid sequence having at least 80% identity (such as at least 85%, 90%, 91%, 92%, 93%, 94%, 95%, 96%, 97%, 98%, 99% or 100% identity) to SEQ ID NO:63; and/or VH2 comprises an amino acid sequence having at least 80% identity (such as at least 85%, 90%, 91%, 92%, 93%, 94%. 95%, 96%, 97%. 98%, 99% or 100% identity) to SEQ ID NO:62. In some aspects, VL2 comprises an amino acid sequence having at least 80% identity (such as at least 85%, 90%, 91%, 92%, 93%, 94%, 95%, 96%, 97%, 98%, 99% or 100% identity) to SEQ ID NO:55; and/or VH2 comprises an amino acid sequence having at least 80% identity (such as at least 85%, 90%, 91%, 92%, 93%, 94%, 95%, 96%, 97%, 98%, 99% or 100% identity) to SEQ ID NO:62. In some aspects, VL2 comprises an amino acid sequence having at least 80% identity (such as at least 85%, 90%, 91%, 92%, 93%, 94%, 95%, 96%, 97%, 98%, 99% or 100% identity) to SEQ ID NO:63; and/or VH2 comprises an amino acid sequence having at least 80% identity (such as at least 85%, 90%, 91%, 92%, 93%, 94%, 95%, 96%, 97%, 98%, 99% or 100% identity) to SEQ ID NO:54.

In some aspects, VL3 and VH3 specifically bind to an immune stimulating receptor (such as CD28). In some aspects, VL3 comprises a CDR1 comprising an amino acid sequence having at least 90% identity, at least 95% identity, or 100% identity to any one of SEQ ID NOs:51, 59, 645, 681, 771 and 843; a CDR2 comprising an amino acid sequence having at least 90% identity, at least 95% identity, or 100% identity to any one of SEQ ID NOs:52, 60, 646, 682, 772 and 844; and a CDR3 comprising an amino acid sequence having at least 90% identity, at least 95% identity, or 100% identity to any one of SEQ ID NOs:53, 61, 647, 683, 773 and 845; and VH3 comprises a CDR1 comprising an amino acid sequence having at least 90% identity, at least 95% identity, or 100% identity to any one of SEQ ID NOs:48, 56, 649, 685, 775 and 847; a CDR2 comprising an amino acid sequence having at least 90% identity, at least 95% identity, or 100% identity to any one of SEQ ID NOs:49, 57, 650, 686, 776 and 848; and a CDR3 comprising an amino acid sequence having at least 90% identity, at least 95% identity, or 100% identity to any one of SEQ ID NOs:50, 58, 651, 687, 777 and 849. In some aspects, VL3 comprises a CDR1 comprising an amino acid sequence having at least 90% identity to SEQ ID NO:51; a CDR2 comprising an amino acid sequence having at least 90% identity to SEQ ID NO:52; and a CDR3 comprising an amino acid sequence having at least 90% identity to SEQ ID NO:53; and VH3 comprises a CDR1 comprising an amino acid sequence having at least 90% identity to SEQ ID NO:48; a CDR2 comprising an amino acid sequence having at least 90% identity to SEQ ID NO:49; and a CDR3 comprising an amino acid sequence having at least 90% identity to SEQ ID NO:50. In some aspects, VL3 comprises a CDR1 comprising an amino acid sequence having at least 90% identity to SEQ ID NO:59; a CDR2 comprising an amino acid sequence having at least 90% identity to SEQ ID NO:60; and a CDR3 comprising an amino acid sequence having at least 90% identity to SEQ ID NO:61; and VH3 comprises a CDR1 comprising an amino acid sequence having at least 90% identity to SEQ ID NO:56; a CDR2 comprising an amino acid sequence having at least 90% identity to SEQ ID NO:57; and a CDR3 comprising an amino acid sequence having at least 90% identity to SEQ ID NO:58. In some aspects, VL3 comprises a CDR1 comprising the amino acid sequence of SEQ ID NO:645; a CDR2 comprising the amino acid sequence of SEQ ID NO:646; and a CDR3 comprising the amino acid sequence of SEQ ID NO:647; and VH3 comprises a CDR1 comprising the amino acid sequence of SEQ ID NO:649; a CDR2 comprising the amino acid sequence of SEQ ID NO:650; and a CDR3 comprising the amino acid sequence of SEQ ID NO:651. In some aspects, VL3 comprises a CDR1 comprising the amino acid sequence of SEQ ID NO:681; a CDR2 comprising the amino acid sequence of SEQ ID NO:682; and a CDR3 comprising the amino acid sequence of SEQ ID NO:683; and VH3 comprises a CDR1 comprising the amino acid sequence of SEQ ID NO:685; a CDR2 comprising the amino acid sequence of SEQ ID NO:686; and a CDR3 comprising the amino acid sequence of SEQ ID NO:687. In some aspects, VL3 comprises a CDR1 comprising the amino acid sequence of SEQ ID NO:771; a CDR2 comprising the amino acid sequence of SEQ ID NO:772; and a CDR3 comprising the amino acid sequence of SEQ ID NO:773; and VH3 comprises a CDR1 comprising the amino acid sequence of SEQ ID NO:775; a CDR2 comprising the amino acid sequence of SEQ ID NO:776; and a CDR3 comprising the amino acid sequence of SEQ ID NO:777. In some aspects, VL3 comprises a CDR1 comprising the amino acid sequence of SEQ ID NO:843; a CDR2 comprising the amino acid sequence of SEQ ID NO:844; and a CDR3 comprising the amino acid sequence of SEQ ID NO:845; and VH3 comprises a CDR1 comprising the amino acid sequence of SEQ ID NO:847; a CDR2 comprising the amino acid sequence of SEQ ID NO:848; and a CDR3 comprising the amino acid sequence of SEQ ID NO:849. As used herein, “at least 90% identity” includes at least 91%, 92%, 93%, 94%, 95%, 96%, 97%, 98%, 99% and 100% identity to the recited reference sequence. In some aspects, VL3 comprises a CDR1 comprising the amino acid sequence of SEQ ID NO:51; a CDR2 comprising the amino acid sequence of SEQ ID NO:52; and a CDR3 comprising the amino acid sequence” of SEQ ID NO:53; and VH3 comprises a CDR1 comprising the amino acid sequence of SEQ ID NO:48; a CDR2 comprising the amino acid sequence of SEQ ID NO:49; and a CDR3 comprising the amino acid sequence of SEQ ID NO:50. In some aspects, VL3 comprises a CDR1 comprising the amino acid sequence of SEQ ID NO:59; a CDR2 comprising the amino acid sequence of SEQ ID NO:60; and a CDR3 comprising the amino acid sequence of SEQ ID NO:61; and VH3 comprises a CDR1 comprising the amino acid sequence of SEQ ID NO:56; a CDR2 comprising the amino acid sequence of SEQ ID NO:57; and a CDR3 comprising the amino acid sequence of SEQ ID NO:58. In some aspects, VL3 comprises a CDR1 comprising an amino acid sequence having at least 90% identity to SEQ ID NO:645; a CDR2 comprising an amino acid sequence having at least 90% identity to SEQ ID NO:646; and a CDR3 comprising an amino acid sequence having at least 90% identity to SEQ ID NO:647; and VH3 comprises a CDR1 comprising an amino acid sequence having at least 90% identity to SEQ ID NO:649; a CDR2 comprising an amino acid sequence having at least 90% identity to SEQ ID NO:650; and a CDR3 comprising an amino acid sequence having at least 90% identity to SEQ ID NO:651. In some aspects, VL3 comprises a CDR1 comprising an amino acid sequence having at least 90% identity to SEQ ID NO:681; a CDR2 comprising an amino acid sequence having at least 90% identity to SEQ ID NO:682; and a CDR3 comprising an amino acid sequence having at least 90% identity to SEQ ID NO:683; and VH3 comprises a CDR1 comprising an amino acid sequence having at least 90% identity to SEQ ID NO:685; a CDR2 comprising an amino acid sequence having at least 90% identity to SEQ ID NO:686; and a CDR3 comprising an amino acid sequence having at least 90% identity to SEQ ID NO:687. In some aspects, VL3 comprises a CDR1 comprising an amino acid sequence having at least 90% identity to SEQ ID NO:771; a CDR2 comprising an amino acid sequence having at least 90% identity to SEQ ID NO:772; and a CDR3 comprising an amino acid sequence having at least 90% identity to SEQ ID NO:773; and VH3 comprises a CDR1 comprising an amino acid sequence having at least 90% identity to SEQ ID NO:775; a CDR2 comprising an amino acid sequence having at least 90% identity to SEQ ID NO:776; and a CDR3 comprising an amino acid sequence having at least 90% identity to SEQ ID NO:777. In some aspects, VL3 comprises a CDR1 comprising an amino acid sequence having at least 90% identity to SEQ ID NO:843; a CDR2 comprising an amino acid sequence having at least 90% identity to SEQ ID NO: 844; and a CDR3 comprising an amino acid sequence having at least 90% identity to SEQ ID NO:845; and VH3 comprises a CDR1 comprising an amino acid sequence having at least 90% identity to SEQ ID NO:847; a CDR2 comprising an amino acid sequence having at least 90% identity to SEQ ID NO: 848; and a CDR3 comprising an amino acid sequence having at least 90% identity to SEQ ID NO:849. In some aspects, VL3 comprises an amino acid sequence having at least 80%, at least 85%, at least 90%, at least 95%, or 100% identity to SEQ ID NO:55 or 63; and VH3 comprises an amino acid sequence having at least 80%, at least 85%, at least 90%, at least 95%, or 100% identity to SEQ ID NO:54 or 62. In some aspects, VL3 comprises an amino acid sequence having at least 80% identity (such as at least 85%, 90%, 91%, 92%, 93%, 94%, 95%, 96%, 97%, 98%, 99% or 100% identity) to SEQ ID NO:55; and/or VH3 comprises an amino acid sequence having at least 80% identity (such as at least 85%, 90%, 91%, 92%, 93%, 94%, 95%, 96%, 97%, 98%, 99% or 100% identity) to SEQ ID NO:54. In some aspects, VL3 comprises an amino acid sequence having at least 80% identity (such as at least 85%, 90%, 91%, 92%, 93%, 94%, 95%, 96%, 97%, 98%, 99% or 100% identity) to SEQ ID NO:63; and/or VH3 comprises an amino acid sequence having at least 80% identity (such as at least 85%, 90%, 91%, 92%, 93%, 94%, 95%, 96%, 97%, 98%, 99% or 100% identity) to SEQ ID NO:62. In some aspects, VL3 comprises an amino acid sequence having at least 80% identity (such as at least 85%, 90%, 91%, 92%, 93%, 94%, 95%, 96%, 97%, 98%, 99% or 100% identity) to SEQ ID NO:55; and/or VH3 comprises an amino acid sequence having at least 80% identity (such as at least 85%, 90%, 91%, 92%, 93%, 94%, 95%, 96%, 97%, 98%, 99% or 100% identity) to SEQ ID NO:62. In some aspects, VL3 comprises an amino acid sequence having at least 80% identity (such as at least 85%, 90%, 91%, 92%, 93%, 94%, 95%, 96%, 97%, 98%, 99% or 100% identity) to SEQ ID NO:63; and/or VH3 comprises an amino acid sequence having at least 80% identity (such as at least 85%, 90%, 91%, 92%, 93%, 94%, 95%, 96%, 97%, 98%, 99% or 100% identity) to SEQ ID NO:54.

In some aspects, VL4 and VH4 specifically bind to an immune stimulating receptor (such as CD28). In some aspects, VL4 comprises a CDR1 comprising an amino acid sequence having at least 90% identity, at least 95% identity, or 100% identity to any one of SEQ ID NOs:51, 59, 645, 681, 771 and 843; a CDR2 comprising an amino acid sequence having at least 90% identity, at least 95% identity, or 100% identity to any one of SEQ ID NOs:52, 60, 646, 682, 772 and 844; and a CDR3 comprising an amino acid sequence having at least 90% identity, at least 95% identity, or 100% identity to any one of SEQ ID NOs:53, 61, 647, 683, 773 and 845; and VH4 comprises a CDR1 comprising an amino acid sequence having at least 90% identity, at least 95% identity, or 100% identity to any one of SEQ ID NOs:48, 56, 649, 685, 775 and 847; a CDR2 comprising an amino acid sequence having at least 90% identity, at least 95% identity, or 100% identity to any one of SEQ ID NOs:49, 57, 650, 686, 776 and 848; and a CDR3 comprising an amino acid sequence having at least 90% identity, at least 95% identity, or 100% identity to any one of SEQ ID NOs:50, 58, 651, 687, 777 and 849. In some aspects, VL4 comprises a CDR1 comprising an amino acid sequence having at least 90% identity to SEQ ID NO:51; a CDR2 comprising an amino acid sequence having at least 90% identity to SEQ ID NO:52; and a CDR3 comprising an amino acid sequence having at least 90% identity to SEQ ID NO:53; and VH4 comprises a CDR1 comprising an amino acid sequence having at least 90% identity to SEQ ID NO:48; a CDR2 comprising an amino acid sequence having at least 90% identity to SEQ ID NO:49; and a CDR3 comprising an amino acid sequence having at least 90% identity to SEQ ID NO:50. In some aspects, VL4 comprises a CDR1 comprising an amino acid sequence having at least 90% identity to SEQ ID NO:59; a CDR2 comprising an amino acid sequence having at least 90% identity to SEQ ID NO:60; and a CDR3 comprising an amino acid sequence having at least 90% identity to SEQ ID NO:61; and VH4 comprises a CDR1 comprising an amino acid sequence having at least 90% identity to SEQ ID NO:56; a CDR2 comprising an amino acid sequence having at least 90% identity to SEQ ID NO:57; and a CDR3 comprising an amino acid sequence having at least 90% identity to SEQ ID NO:58. In some aspects, VL4 comprises a CDR1 comprising an amino acid sequence having at least 90% identity to SEQ ID NO:645; a CDR2 comprising an amino acid sequence having at least 90% identity to SEQ ID NO:646; and a CDR3 comprising an amino acid sequence having at least 90% identity to SEQ ID NO:647; and VH4 comprises a CDR1 comprising an amino acid sequence having at least 90% identity to SEQ ID NO:649; a CDR2 comprising an amino acid sequence having at least 90% identity to SEQ ID NO:650; and a CDR3 comprising an amino acid sequence having at least 90% identity to SEQ ID NO:651. In some aspects, VL4 comprises a CDR1 comprising an amino acid sequence having at least 90% identity to SEQ ID NO:681; a CDR2 comprising an amino acid sequence having at least 90% identity to SEQ ID NO:682; and a CDR3 comprising an amino acid sequence having at least 90% identity to SEQ ID NO:683; and VH4 comprises a CDR1 comprising an amino acid sequence having at least 90% identity to SEQ ID NO:685; a CDR2 comprising an amino acid sequence having at least 90% identity to SEQ ID NO:686; and a CDR3 comprising an amino acid sequence having at least 90% identity to SEQ ID NO:687. In some aspects, VL4 comprises a CDR1 comprising an amino acid sequence having at least 90% identity to SEQ ID NO:771; a CDR2 comprising an amino acid sequence having at least 90% identity to SEQ ID NO:772; and a CDR3 comprising an amino acid sequence having at least 90% identity to SEQ ID NO:773; and VH4 comprises a CDR1 comprising an amino acid sequence having at least 90% identity to SEQ ID NO:775; a CDR2 comprising an amino acid sequence having at least 90% identity to SEQ ID NO:776; and a CDR3 comprising an amino acid sequence having at least 90% identity to SEQ ID NO:777. In some aspects, VL4 comprises a CDR1 comprising an amino acid sequence having at least 90% identity to SEQ ID NO:843; a CDR2 comprising an amino acid sequence having at least 90% identity to SEQ ID NO: 844; and a CDR3 comprising an amino acid sequence having at least 90% identity to SEQ ID NO:845; and VH4 comprises a CDR1 comprising an amino acid sequence having at least 90% identity to SEQ ID NO:847; a CDR2 comprising an amino acid sequence having at least 90% identity to SEQ ID NO:848; and a CDR3 comprising an amino acid sequence having at least 90% identity to SEQ ID NO:849. As used herein, “at least 90% identity” includes at least 91%, 92%, 93%, 94%, 95%, 96%, 97%, 98%, 99% and 100% identity to the recited reference sequence. In some aspects, VL4 comprises a CDR1 comprising the amino acid sequence of SEQ ID NO:51; a CDR2 comprising the amino acid sequence of SEQ ID NO:52; and a CDR3 comprising the amino acid sequence of SEQ ID NO:53; and VH4 comprises a CDR1 comprising the amino acid sequence of SEQ ID NO:48; a CDR2 comprising the amino acid sequence of SEQ ID NO:49; and a CDR3 comprising the amino acid sequence of SEQ ID NO:50 In some aspects, VL4 comprises a CDR1 comprising the amino acid sequence of SEQ ID NO:59; a CDR2 comprising the amino acid sequence of SEQ ID NO:60; and a CDR3 comprising the amino acid sequence of SEQ ID NO:61; and VH4 comprises a CDR1 comprising the amino acid sequence of SEQ ID NO:56; a CDR2 comprising the amino acid sequence of SEQ ID NO:57; and a CDR3 comprising the amino acid sequence of SEQ ID NO:58. In some aspects, VL4 comprises a CDR1 comprising the amino acid sequence of SEQ ID NO:645; a CDR2 comprising the amino acid sequence of SEQ ID NO:646; and a CDR3 comprising the amino acid sequence of SEQ ID NO:647; and VH4 comprises a CDR1 comprising the amino acid sequence of SEQ ID NO:649; a CDR2 comprising the amino acid sequence of SEQ ID NO:650; and a CDR3 comprising the amino acid sequence of SEQ ID NO:651. In some aspects, VL4 comprises a CDR1 comprising the amino acid sequence of SEQ ID NO:681; a CDR2 comprising the amino acid sequence of SEQ ID NO:682; and a CDR3 comprising the amino acid sequence of SEQ ID NO:683; and VH4 comprises a CDR1 comprising the amino acid sequence of SEQ ID NO:685; a CDR2 comprising the amino acid sequence of SEQ ID NO:686; and a CDR3 comprising the amino acid sequence of SEQ ID NO:687. In some aspects, VL4 comprises a CDR1 comprising the amino acid sequence of SEQ ID NO:771; a CDR2 comprising the amino acid sequence of SEQ ID NO:772; and a CDR3 comprising the amino acid sequence of SEQ ID NO:773; and VH4 comprises a CDR1 comprising the amino acid sequence of SEQ ID NO:775; a CDR2 comprising the amino acid sequence of SEQ ID NO:776; and a CDR3 comprising the amino acid sequence of SEQ ID NO:777. In some aspects, VL4 comprises a CDR1 comprising the amino acid sequence of SEQ ID NO:843; a CDR2 comprising the amino acid sequence of SEQ ID NO:844; and a CDR3 comprising the amino acid sequence of SEQ ID NO:845; and VH4 comprises a CDR1 comprising the amino acid sequence of SEQ ID NO:847; a CDR2 comprising the amino acid sequence of SEQ ID NO:848; and a CDR3 comprising the amino acid sequence of SEQ ID NO:849. In some aspects, VL4 comprises an amino acid sequence having at least 80%, at least 85%, at least 90%, at least 95%, or 100% identity to SEQ ID NO:55 or 63; and VH4 comprises an amino acid sequence having at least 80%, at least 85%, at least 90%, at least 95%, or 100% identity to SEQ ID NO:54 or 62. In some aspects, VL4 comprises an amino acid sequence having at least 80% identity (such as at least 85%, 90%, 91%, 92%, 93%, 94%, 95%, 96%, 97%, 98%, 99% or 100% identity) to SEQ ID NO:55; and/or VH4 comprises an amino acid sequence having at least 80% identity (such as at least 85%, 90%, 91%, 92%, 93%, 94%, 95%, 96%, 97%, 98%, 99% or 100% identity) to SEQ ID NO:54. In some aspects, VL4 comprises an amino acid sequence having at least 80% identity (such as at least 85%, 90%, 91%, 92%, 93%, 94%, 95%, 96%, 97%, 98%, 99% or 100% identity) to SEQ ID NO:63; and/or VH4 comprises an amino acid sequence having at least 80% identity (such as at least 85%, 90%, 91%, 92%, 93%, 94%, 95%, 96%, 97%, 98%, 99% or 100% identity) to SEQ ID NO:62. In some aspects, VL4 comprises an amino acid sequence having at least 80% identity (such as at least 85%, 90%, 91%, 92%, 93%, 94%, 95%, 96%, 97%, 98%, 99% or 100% identity) to SEQ ID NO:55; and/or VH4 comprises an amino acid sequence having at least 80% identity (such as at least 85%, 90%, 91%, 92%, 93%, 94%, 95%, 96%, 97%, 98%, 99% or 100% identity) to SEQ ID NO:62. In some aspects, VL4 comprises an amino acid sequence having at least 80% identity (such as at least 85%, 90%, 91%, 92%, 93%, 94%, 95%, 96%, 97%, 98%, 99% or 100% identity) to SEQ ID NO:63; and/or VH4 comprises an amino acid sequence having at least 80% identity (such as at least 85%, 90%, 91%, 92%, 93%, 94%, 95%, 96%, 97%, 98%, 99% or 100% identity) to SEQ ID NO:54.

In some aspects, the antigen binding polypeptide complex (e.g., antibody or antigen binding fragment thereof) of the invention specifically binds to CD28. In some aspects, the antigen binding polypeptide complex specifically binds to CD28 and CD3. In some aspects, the antigen binding polypeptide complex specifically binds to CD28, CD3, and one or more TAAs, such as, e.g., A2AR, APRIL, ATPDase, BAFF, BAFFR, BCMA, BlyS, BTK, BTLA, B7DC, B7H1, B7H2, B7H3, B7H4, B7H5, B7H6, B7H7, B7RP1, B7-4, C3, C5, CCL2, CCL3, CCL4, CCL5, CCL7, CCL8, CCL11, CCL15, CCL17, CCL19, CCL20, CCL21, CCL25, CCR3, CCR4, CD16A, CD19, CD20, CD24, CD27, CD28, CD30, CD38, CD39, CD40, CD40L, CD47, CD52, CD70, CD80, CD86, CD123, CD133, CD137, CD137L, CD160, CD272, CEACAM5, CLEC9, CLEC91, CRTH2, CSF-1, CSF-2, CSF-3, CXCL1, CXCL2, CXCL4, CXCL12, CXCL13, CXCR3, cMet, CTLA4, DLL3, DLL4, DNGR-1, E-cadherin, EGFR, ENTPD1, EpCAM, FCER1, FCER1A, FCER2, FGFR, FLAP, FOLH1, Gi24, GITR, GITRL, GPR5, GP100, GPRC5D, HER2, HER3, ICOSL, ICOS, HHLA2, HMGB1, HVEM, IDO, IFNa, IgE, IGF1R, IL2Rbeta, IL1, ILIA, IL1B, IL1F10, IL2, IL4, IL4Ra, IL5, IL5R, IL6, IL7, IL7Ra, IL8, IL9, IL9R, IL10, rhILlO, IL12, IL13, IL13Ra1, IL13Ra2, IL15, IL17, IL17Rb, IL18, IL22, IL23, IL25, IL7, IL33, IL35, ITGB4, ITK, KIR, LAG3, LAMP1, leptin, LPFS2, MHC class II, MUC-1, MUC-16, NCR3LG1, NKG2D, NKp46, NTPDase-1, OX40, OX40L, PD-1, PD-L1, PD-L2, PROM1, S152, SIRPalpha, SISP1, SLC, SPG64, ST2, STEAP1, STEAP2, Syk kinase, STEAP1, TROP2, TACI, TDO, TGFBETA, T14, TIGIT, TIM3, TLR, TLR2, TLR4, TLR5, TLR9, TMEF1, TNFa, TNFRSF7, Tp55, TREM1, TSLP, TSLPR, TWEAK, VEGF, VISTA, Vstm3, or WUCAM. For example, the antigen binding polypeptide complex may specifically bind CD3, CD28, CD19 and CD20. For example, the antigen binding polypeptide complex may specifically bind CD3, CD28, cMet and Trop2.

In some aspects, a VL and a corresponding VH of the antigen binding polypeptide complex specifically bind to CD28. In some aspects, one VL and one corresponding VH of the antigen binding polypeptide complex specifically bind to CD28. In some aspects, VL1 and VH1 specifically bind to CD28. In some aspects, VL1 comprises a CDR1 comprising an amino acid sequence having at least 90% identity, at least 95% identity, or 100% identity to any one of SEQ ID NOs:51, 59, 645, 681, 771 and 843; a CDR2 comprising an amino acid sequence having at least 90% identity, at least 95% identity, or 100% identity to any one of SEQ ID NOs:52, 60, 646, 682, 772 and 844; and a CDR3 comprising an amino acid sequence having at least 90% identity, at least 95% identity, or 100% identity to any one of SEQ ID NOs:53, 61, 647, 683, 773 and 845; and VH1 comprises a CDR1 comprising an amino acid sequence having at least 90% identity, at least 95% identity, or 100% identity to any one of SEQ ID NOs:48, 56, 649, 685, 775 and 847; a CDR2 comprising an amino acid sequence having at least 90% identity, at least 95% identity, or 100% identity to any one of SEQ ID NOs:49, 57, 650, 686, 776 and 848; and a CDR3 comprising an amino acid sequence having at least 90% identity, at least 95% identity, or 100% identity to any one of SEQ ID NOs:50, 58, 651, 687, 777 and 849. In some aspects, VL1 comprises an amino acid sequence having at least 80%, at least 85%, at least 90%, at least 95%, or 100% identity to any one of SEQ ID NOs:55, 63, 527, 644, 680, 770 and 842; and VH1 comprises an amino acid sequence having at least 80%, at least 85%, at least 90%, at least 95%, or 100% identity to any one of SEQ ID NOs:54, 62, 528, 648, 684, 774 and 846.

In some aspects, VL2 and VH2 specifically bind to CD28. In some aspects, VL2 comprises a CDR1 comprising an amino acid sequence having at least 90% identity, at least 95% identity, or 100% identity to any one of SEQ ID NOs:51, 59, 645, 681, 771 and 843; a CDR2 comprising an amino acid sequence having at least 90% identity, at least 95% identity, or 100% identity to any one of SEQ ID NOs:52, 60, 646, 682, 772 and 844; and a CDR3 comprising an amino acid sequence having at least 90% identity, at least 95% identity, or 100% identity to any one of SEQ ID NOs:53, 61, 647, 683, 773 and 845; and VH2 comprises a CDR1 comprising an amino acid sequence having at least 90% identity, at least 95% identity, or 100% identity to any one of SEQ ID NOs:48, 56, 649, 685, 775 and 847; a CDR2 comprising an amino acid sequence having at least 90% identity, at least 95% identity, or 100% identity to any one of SEQ ID NOs:49, 57, 650, 686, 776 and 848; and a CDR3 comprising an amino acid sequence having at least 90% identity, at least 95% identity, or 100% identity to any one of SEQ ID NOs:50, 58, 651, 687, 777 and 849. In some aspects, VL2 comprises an amino acid sequence having at least 80%, at least 85%, at least 90%, at least 95%, or 100% identity to any one of SEQ ID NOs:55, 63, 527, 644, 680, 770 and 842; and VH2 comprises an amino acid sequence having at least 80%, at least 85%, at least 90%, at least 95%, or 100% identity to any one of SEQ ID NOs:54, 62, 528, 648, 684, 774 and 846.

In some aspects, VL3 and VH3 specifically bind to CD28. In some aspects, VL3 comprises a CDR1 comprising an amino acid sequence having at least 90% identity, at least 95% identity, or 100% identity to any one of SEQ ID NOs:51, 59, 645, 681, 771 and 843; a CDR2 comprising an amino acid sequence having at least 90% identity, at least 95% identity, or 100% identity to ay one of SEQ ID NOs:52, 60, 646, 682, 772, 844; and a CDR3 comprising an amino acid sequence having at least 90% identity, at least 95% identity, or 100% identity to any one of SEQ ID NOs:53, 61, 647, 683, 773 and 845; and VH3 comprises a CDR1 comprising an amino acid sequence having at least 90% identity, at least 95% identity, or 100% identity to any one of SEQ ID NOs:48, 56, 649, 685, 775 and 847; a CDR2 comprising an amino acid sequence having at least 90% identity, at least 95% identity, or 100% identity to any one of SEQ ID NOs:49, 57, 650, 686, 776 and 848; and a CDR3 comprising an amino acid sequence having at least 90% identity, at least 95% identity, or 100% identity to any one of SEQ ID NOs:50, 58, 651, 687, 777 and 849. In some aspects, VL3 comprises an amino acid sequence having at least 80%, at least 85%, at least 90%, at least 95%, or 100% identity to any one of SEQ ID NOs:55, 63, 527, 644, 680, 770 and 842; and VH3 comprises an amino acid sequence having at least 80%, at least 85%, at least 90%, at least 95%, or 100% identity to any one of SEQ ID NOs:54, 62, 528, 648, 684, 774 and 846.

In some aspects, VL4 and VH4 specifically bind to CD28. In some aspects, VL4 comprises a CDR1 comprising an amino acid sequence having at least 90% identity, at least 95% identity, or 100% identity to any one of SEQ ID NOs:51, 59, 645, 681, 771 and 843; a CDR2 comprising an amino acid sequence having at least 90% identity, at least 95% identity, or 100% identity to any one of SEQ ID NOs:52, 60, 646, 682, 772 and 844; and a CDR3 comprising an amino acid sequence having at least 90% identity, at least 95% identity, or 100% identity to any one of SEQ ID NOs:53, 61, 647, 683, 773 and 845; and VH4 comprises a CDR1 comprising an amino acid sequence having at least 90% identity, at least 95% identity, or 100% identity to any one of SEQ ID NOs:48, 56, 649, 685, 775 and 847; a CDR2 comprising an amino acid sequence having at least 90% identity, at least 95% identity, or 100% identity to any one of SEQ ID NOs:49, 57, 650, 686, 776 and 848; and a CDR3 comprising an amino acid sequence having at least 90% identity, at least 95% identity, or 100% identity to any one of SEQ ID NOs:50, 58, 651, 687, 777 and 849. In some aspects, VL4 comprises an amino acid sequence having at least 80%, at least 85%, at least 90%, at least 95%, or 100% identity to any one of SEQ ID NOs:55, 63, 527, 644, 680, 770 and 842; and VH4 comprises an amino acid sequence having at least 80%, at least 85%, at least 90%, at least 95%, or 100% identity to any one of SEQ ID NOs:54, 62, 528, 648, 684, 774 and 846.

In some aspects, the invention is directed to an antigen binding polypeptide complex comprising a first polypeptide, a second polypeptide, and a third polypeptide; wherein the first polypeptide has a structure represented by VL1-L1-VH1-L2-Fc; or VH1-L3-VL1-L4-Fc; wherein the second polypeptide has a structure represented by VH2-L5-VH3-L6-CH1-L7-Fc; wherein the third polypeptide has a structure represented by VL2-L8-VL3-L9-CL; wherein VL1 is a first immunoglobulin light chain variable region that specifically binds to a TAA; VL2 is a second immunoglobulin light chain variable region that specifically binds to a TAA; VL3 is a third immunoglobulin light chain variable region that specifically binds to human CD3; VH1 is a first immunoglobulin heavy chain variable region that specifically binds to a TAA; VH2 is a second immunoglobulin heavy chain variable region that specifically binds to a TAA; VH3 is a third immunoglobulin heavy chain variable region that specifically binds to human CD3; Fc is a region comprising an immunoglobulin heavy chain constant region 2 (CH2), an immunoglobulin heavy chain constant region 3 (CH3), and optionally, an immunoglobulin hinge; CH1 is an immunoglobulin heavy chain constant region 1; CL is an immunoglobulin light chain constant region; and L1-L9 are amino acid linkers. In some aspects, the TAA bound by VL1 and VH1 is CD19 and the TAA bound by VL2 and VH2 is CD20. In some aspects, the TAA bound by VL1 and VH1 is CD20 and the TAA bound by VL2 and VH2 is CD19. In some aspects, the TAA bound by VL1 and VH1 is cMet and the TAA bound by VL2 and VH2 is Trop2. In some aspects, the TAA bound by VL1 and VH1 is Trop2 and the TAA bound by VL2 and VH2 is cMet.

In some aspects, the invention is directed to an antigen binding polypeptide complex comprising a first polypeptide, a second polypeptide, and a third polypeptide; wherein the first polypeptide has a structure represented by VL1-L1-VH1-L2-Fc; or VH1-L3-VL1-L4-Fc; wherein the second polypeptide has a structure represented by VH2-L5-VH3-L6-CH1-L7-Fc; wherein the third polypeptide has a structure represented by VL2-L8-VL3-L9-CL; wherein VL1 is a first immunoglobulin light chain variable region that specifically binds to an immune stimulating receptor; VL2 is a second immunoglobulin light chain variable region that specifically binds to a TAA; VL3 is a third immunoglobulin light chain variable region that specifically binds to human CD3; VH1 is a first immunoglobulin heavy chain variable region that specifically binds to an immune stimulating receptor; VH2 is a second immunoglobulin heavy chain variable region that specifically binds to a TAA; VH3 is a third immunoglobulin heavy chain variable region that specifically binds to human CD3; Fc is a region comprising an immunoglobulin heavy chain constant region 2 (CH2), an immunoglobulin heavy chain constant region 3 (CH3), and optionally, an immunoglobulin hinge; CH1 is an immunoglobulin heavy chain constant region 1; CL is an immunoglobulin light chain constant region; and L1-L9 are amino acid linkers. In some aspects, the immune stimulating receptor bound by VL1 and VH1 is CD28 and/or the TAA bound by VL2 and VH2 is CD19. In some aspects, the immune stimulating receptor bound by VL1 and VH1 is CD28 and/or the TAA bound by VL2 and VH2 is CD20. In some aspects, the immune stimulating receptor bound by VL1 and VH1 is CD28 and/or the TAA bound by VL2 and VH2 is cMet. In some aspects, the immune stimulating receptor bound by VL1 and VH1 is CD28 and/or the TAA bound by VL2 and VH2 is Trop2.

In some aspects, the invention is directed to an antigen binding polypeptide complex comprising a first polypeptide, a second polypeptide, and a third polypeptide; wherein the first polypeptide has a structure represented by VL1-L1-VH1-L2-Fc; or VH1-L3-VL1-L4-Fc; wherein the second polypeptide has a structure represented by VH2-L5-VH3-L6-CH1-L7-Fc; wherein the third polypeptide has a structure represented by VL2-L8-VL3-L9-CL; wherein VL1 is a first immunoglobulin light chain variable region that specifically binds to CD28; VL2 is a second immunoglobulin light chain variable region that specifically binds to a TAA; VL3 is a third immunoglobulin light chain variable region that specifically binds to human CD3; VH1 is a first immunoglobulin heavy chain variable region that specifically binds to a CD28; VH2 is a second immunoglobulin heavy chain variable region that specifically binds to a TAA; VH3 is a third immunoglobulin heavy chain variable region that specifically binds to human CD3; Fc is a region comprising an immunoglobulin heavy chain constant region 2 (CH2), an immunoglobulin heavy chain constant region 3 (CH3), and optionally, an immunoglobulin hinge; CH1 is an immunoglobulin heavy chain constant region 1; CL is an immunoglobulin light chain constant region; and L1-L9 are amino acid linkers. In some aspects, VL2 and VH2 specifically binds to CD19. In some aspects, VL2 and VH2 specifically binds to CD20. In some aspects, VL2 and VH2 specifically binds to cMet. In some aspects, VL2 and VH2 specifically binds to Trop2.

In some aspects, the invention is directed to an antigen binding polypeptide complex comprising a first polypeptide, a second polypeptide, and a third polypeptide; wherein the first polypeptide has a structure represented by VL1-L1-VH1-L2-Fc; or VH1-L3-VL1-L4-Fc; wherein the second polypeptide has a structure represented by VH2-L5-VH3-L6-CH1-L7-Fc; wherein the third polypeptide has a structure represented by VL2-L8-VL3-L9-CL; wherein VL1 is a first immunoglobulin light chain variable region that specifically binds to a TAA; VL2 is a second immunoglobulin light chain variable region that specifically binds to an immune stimulating receptor; VL3 is a third immunoglobulin light chain variable region that specifically binds to human CD3; VH1 is a first immunoglobulin heavy chain variable region that specifically binds to a TAA; VH2 is a second immunoglobulin heavy chain variable region that specifically binds to an immune stimulating receptor; VH3 is a third immunoglobulin heavy chain variable region that specifically binds to human CD3; Fc is a region comprising an immunoglobulin heavy chain constant region 2 (CH2), an immunoglobulin heavy chain constant region 3 (CH3), and optionally, an immunoglobulin hinge; CH1 is an immunoglobulin heavy chain constant region 1; CL is an immunoglobulin light chain constant region; and L1-L9 are amino acid linkers. In some aspects, the TAA bound by VL1 and VH1 is CD19 and/or the immune stimulating receptor bound by VL2 and VH2 is CD28. In some aspects, the TAA bound by VL1 and VH1 is CD20 and/or the immune stimulating receptor bound by VL2 and VH2 is CD28. In some aspects, the TAA bound by VL1 and VH1 is cMet and/or the immune stimulating receptor bound by VL2 and VH2 is CD28. In some aspects, the TAA bound by VL1 and VH1 is Trop2 and/or the immune stimulating receptor bound by VL2 and VH2 is CD28.

In some aspects, the invention is directed to an antigen binding polypeptide complex comprising a first polypeptide, a second polypeptide, and a third polypeptide; wherein the first polypeptide has a structure represented by VL1-L1-VH1-L2-Fc; or VH1-L3-VL1-L4-Fc; wherein the second polypeptide has a structure represented by VH2-L5-VH3-L6-CH1-L7-Fc; wherein the third polypeptide has a structure represented by VL2-L8-VL3-L9-CL; wherein VL1 is a first immunoglobulin light chain variable region that specifically binds to a TAA; VL2 is a second immunoglobulin light chain variable region that specifically binds to CD28; VL3 is a third immunoglobulin light chain variable region that specifically binds to human CD3; VH1 is a first immunoglobulin heavy chain variable region that specifically binds to a TAA; VH2 is a second immunoglobulin heavy chain variable region that specifically binds to CD28; VH3 is a third immunoglobulin heavy chain variable region that specifically binds to human CD3; Fc is a region comprising an immunoglobulin heavy chain constant region 2 (CH2), an immunoglobulin heavy chain constant region 3 (CH3), and optionally, an immunoglobulin hinge; CH1 is an immunoglobulin heavy chain constant region 1; CL is an immunoglobulin light chain constant region; and L1-L9 are amino acid linkers. In some aspects, VL1 and VH1 specifically binds to CD19. In some aspects, VL1 and VH1 specifically binds to CD20. In some aspects, VL1 and VH1 specifically binds to cMet. In some aspects, VL1 and VH1 specifically binds to Trop2.

In some aspects, the invention is directed to an antigen binding polypeptide complex comprising a first polypeptide, a second polypeptide, a third polypeptide, and a fourth polypeptide; wherein the first polypeptide has a structure represented by VL1-L1-CL; wherein the second polypeptide has a structure represented by VH1-L2-CH1-L3-Fc; wherein the third polypeptide has a structure represented by VH2-L4-VH3-L5-CH1-L6-Fc; wherein the fourth polypeptide has a structure represented by VL2-L7-VL3-L8-CL; wherein VL1 is a first immunoglobulin light chain variable region that specifically binds to a TAA; VL2 is a second immunoglobulin light chain variable region that specifically binds to a TAA; VL3 is a third immunoglobulin light chain variable region that specifically binds to human CD3; VH1 is a first immunoglobulin heavy chain variable region that specifically binds to a TAA; VH2 is a second immunoglobulin heavy chain variable region that specifically binds to a TAA; VH3 is a third immunoglobulin heavy chain variable region that specifically binds to human CD3; Fc is a region comprising an immunoglobulin heavy chain constant region 2 (CH2), an immunoglobulin heavy chain constant region 3 (CH3), and optionally, an immunoglobulin hinge; CH1 is an immunoglobulin heavy chain constant region 1; CL is an immunoglobulin light chain constant region; and L1-L8 are amino acid linkers. In some aspects, the TAA bound by VL1 and VH1 is CD19 and the TAA bound by VL2 and VH2 is CD20. In some aspects, the TAA bound by VL1 and VH1 is CD20 and the TAA bound by VL2 and VH2 is CD19. In some aspects, the TAA bound by VL1 and VH1 is cMet and the TAA bound by VL2 and VH2 is Trop2. In some aspects, the TAA bound by VL1 and VH1 is Trop2 and the TAA bound by VL2 and VH2 is cMet.

In some aspects, the invention is directed to an antigen binding polypeptide complex comprising a first polypeptide, a second polypeptide, a third polypeptide, and a fourth polypeptide; wherein the first polypeptide has a structure represented by VL1-L1-CL; wherein the second polypeptide has a structure represented by VH1-L2-CH1-L3-Fc; wherein the third polypeptide has a structure represented by VH2-L4-VH3-L5-CH1-L6-Fc; wherein the fourth polypeptide has a structure represented by VL2-L7-VL3-L8-CL; wherein VL1 is a first immunoglobulin light chain variable region that specifically binds to an immune stimulating receptor; VL2 is a second immunoglobulin light chain variable region that specifically binds to a TAA; VL3 is a third immunoglobulin light chain variable region that specifically binds to human CD3; VH1 is a first immunoglobulin heavy chain variable region that specifically binds to an immune stimulating receptor; VH2 is a second immunoglobulin heavy chain variable region that specifically binds to a TAA; VH3 is a third immunoglobulin heavy chain variable region that specifically binds to human CD3; Fc is a region comprising an immunoglobulin heavy chain constant region 2 (CH2), an immunoglobulin heavy chain constant region 3 (CH3), and optionally, an immunoglobulin hinge; CH1 is an immunoglobulin heavy chain constant region 1; CL is an immunoglobulin light chain constant region; and L1-L8 are amino acid linkers. In some aspects, the immune stimulating receptor bound by VL1 and VH1 is CD28 and/or the TAA bound by VL2 and VH2 is CD19. In some aspects, the immune stimulating receptor bound by VL1 and VH1 is CD28 and/or the TAA bound by VL2 and VH2 is CD20. In some aspects, the immune stimulating receptor bound by VL1 and VH1 is CD28 and/or the TAA bound by VL2 and VH2 is cMet. In some aspects, the immune stimulating receptor bound by VL1 and VH1 is CD28 and/or the TAA bound by VL2 and VH2 is Trop2.

In some aspects, the invention is directed to an antigen binding polypeptide complex comprising a first polypeptide, a second polypeptide, a third polypeptide, and a fourth polypeptide; wherein the first polypeptide has a structure represented by VL1-L1-CL; wherein the second polypeptide has a structure represented by VH1-L2-CH1-L3-Fc; wherein the third polypeptide has a structure represented by VH2-L4-VH3-L5-CH1-L6-Fc; wherein the fourth polypeptide has a structure represented by VL2-L7-VL3-L8-CL; wherein VL1 is a first immunoglobulin light chain variable region that specifically binds to CD28; VL2 is a second immunoglobulin light chain variable region that specifically binds to a TAA; VL3 is a third immunoglobulin light chain variable region that specifically binds to human CD3; VH1 is a first immunoglobulin heavy chain variable region that specifically binds to CD28; VH2 is a second immunoglobulin heavy chain variable region that specifically binds to a TAA; VH3 is a third immunoglobulin heavy chain variable region that specifically binds to human CD3; Fc is a region comprising an immunoglobulin heavy chain constant region 2 (CH2), an immunoglobulin heavy chain constant region 3 (CH3), and optionally, an immunoglobulin hinge; CH1 is an immunoglobulin heavy chain constant region 1; CL is an immunoglobulin light chain constant region; and L1-L8 are amino acid linkers. In some aspects, VL2 and VH2 specifically binds to CD19. In some aspects, VL2 and VH2 specifically binds to CD20. In some aspects, VL2 and VH2 specifically binds to cMet. In some aspects, VL2 and VH2 specifically binds to Trop2.

In some aspects, the invention is directed to an antigen binding polypeptide complex comprising a first polypeptide, a second polypeptide, a third polypeptide, and a fourth polypeptide; wherein the first polypeptide has a structure represented by VL1-L1-CL; wherein the second polypeptide has a structure represented by VH1-L2-CH1-L3-Fc; wherein the third polypeptide has a structure represented by VH2-L4-VH3-L5-CH1-L6-Fc; wherein the fourth polypeptide has a structure represented by VL2-L7-VL3-L8-CL; wherein VL1 is a first immunoglobulin light chain variable region that specifically binds to a TAA; VL2 is a second immunoglobulin light chain variable region that specifically binds to an immune stimulating receptor; VL3 is a third immunoglobulin light chain variable region that specifically binds to human CD3; VH1 is a first immunoglobulin heavy chain variable region that specifically binds to a TAA; VH2 is a second immunoglobulin heavy chain variable region that specifically binds to an immune stimulating receptor; VH3 is a third immunoglobulin heavy chain variable region that specifically binds to human CD3; Fc is a region comprising an immunoglobulin heavy chain constant region 2 (CH2), an immunoglobulin heavy chain constant region 3 (CH3), and optionally, an immunoglobulin hinge; CH1 is an immunoglobulin heavy chain constant region 1; CL is an immunoglobulin light chain constant region; and L1-L8 are amino acid linkers. In some aspects, the TAA bound by VL1 and VH1 is CD19 and/or the immune stimulating receptor bound by VL2 and VH2 is CD28. In some aspects, the TAA bound by VL1 and VH1 is CD20 and/or the immune stimulating receptor bound by VL2 and VH2 is CD28. In some aspects, the TAA bound by VL1 and VH1 is cMet and/or the immune stimulating receptor bound by VL2 and VH2 is CD28. In some aspects, the TAA bound by VL1 and VH1 is Trop2 and/or the immune stimulating receptor bound by VL2 and VH2 is CD28.

In some aspects, the invention is directed to an antigen binding polypeptide complex comprising a first polypeptide, a second polypeptide, a third polypeptide, and a fourth polypeptide; wherein the first polypeptide has a structure represented by VL1-L1-CL; wherein the second polypeptide has a structure represented by VH1-L2-CH1-L3-Fc; wherein the third polypeptide has a structure represented by VH2-L4-VH3-L5-CH1-L6-Fc; wherein the fourth polypeptide has a structure represented by VL2-L7-VL3-L8-CL; wherein VL1 is a first immunoglobulin light chain variable region that specifically binds to a TAA; VL2 is a second immunoglobulin light chain variable region that specifically binds to CD28; VL3 is a third immunoglobulin light chain variable region that specifically binds to human CD3; VH1 is a first immunoglobulin heavy chain variable region that specifically binds to a TAA; VH2 is a second immunoglobulin heavy chain variable region that specifically binds to CD28; VH3 is a third immunoglobulin heavy chain variable region that specifically binds to human CD3; Fc is a region comprising an immunoglobulin heavy chain constant region 2 (CH2), an immunoglobulin heavy chain constant region 3 (CH3), and optionally, an immunoglobulin hinge; CH1 is an immunoglobulin heavy chain constant region 1; CL is an immunoglobulin light chain constant region; and L1-L8 are amino acid linkers. In some aspects, VL1 and VH1 specifically binds to CD19. In some aspects, VL1 and VH1 specifically binds to CD20. In some aspects, VL1 and VH1 specifically binds to cMet. In some aspects, VL1 and VH1 specifically binds to Trop2.

In some aspects, the invention is directed to an antigen binding polypeptide complex comprising a first polypeptide, a second polypeptide, and a third polypeptide; wherein the first polypeptide has a structure represented by VL1-L1-VL2-L2-VH2-L3-VH1-L4-Fc; or VH1-L5-VH2-L6-VL2-L7-VL1-L8-Fc; wherein the second polypeptide has a structure represented by VH3-L9-VH4-L10-CH1-L11-Fc; wherein the third polypeptide has a structure represented by VL3-L12-VL4-L13-CL; wherein VL1 is a first immunoglobulin light chain variable region that specifically binds to a TAA; VL2 is a second immunoglobulin light chain variable region that specifically binds to a TAA; VL3 is a third immunoglobulin light chain variable region that specifically binds to CD28; VL4 is a fourth immunoglobulin light chain variable region that specifically binds to human CD3; VH1 is a first immunoglobulin heavy chain variable region that specifically binds to a TAA; VH2 is a second immunoglobulin heavy chain variable region that specifically binds to a TAA; VH3 is a third immunoglobulin heavy chain variable region that specifically binds to CD28; VH4 is a fourth immunoglobulin heavy chain variable region that specifically binds to human CD3; Fc is a region comprising an immunoglobulin heavy chain constant region 2 (CH2), an immunoglobulin heavy chain constant region 3 (CH3), and optionally, an immunoglobulin hinge; CH1 is an immunoglobulin heavy chain constant region 1; CL is an immunoglobulin light chain constant region; and L1-L13 are amino acid linkers. In some aspects, the invention is directed to an antigen binding polypeptide complex comprising a first polypeptide, a second polypeptide, and a third polypeptide; wherein the first polypeptide has a structure represented by VL1-L1-VL2-L2-VH2-L3-VH1-L4-Fc; or VH1-L5-VL2-L6-VH2-L7-VL1-L8-Fc; wherein the second polypeptide has a structure represented by VH3-L9-VH4-L10-CH1-L11-Fc; wherein the third polypeptide has a structure represented by VL3-L12-VL4-L13-CL; wherein VL1 is a first immunoglobulin light chain variable region that specifically binds to a TAA; VL2 is a second immunoglobulin light chain variable region that specifically binds to a TAA; VL3 is a third immunoglobulin light chain variable region that specifically binds to CD28; VL4 is a fourth immunoglobulin light chain variable region that specifically binds to human CD3; VH1 is a first immunoglobulin heavy chain variable region that specifically binds to a TAA; VH2 is a second immunoglobulin heavy chain variable region that specifically binds to a TAA; VH3 is a third immunoglobulin heavy chain variable region that specifically binds to CD28; VH4 is a fourth immunoglobulin heavy chain variable region that specifically binds to human CD3; Fc is a region comprising an immunoglobulin heavy chain constant region 2 (CH2), an immunoglobulin heavy chain constant region 3 (CH3), and optionally, an immunoglobulin hinge; CH1 is an immunoglobulin heavy chain constant region 1; CL is an immunoglobulin light chain constant region; and L1-L13 are amino acid linkers. In some aspects, the first polypeptide has a structure represented by VL1-L1-VL2-L2-VH2-L3-VH1-L4-Fc; the second polypeptide has a structure represented by VH3-L9-VH4-L10-CH1-L11-Fc; and the third polypeptide has a structure represented by VL3-L12-VL4-L13-CL. In some aspects, the first polypeptide has a structure represented by VH1-L5-VL2-L6-VH2-L7-VL1-L8-Fc; the second polypeptide has a structure represented by VH3-L9-VH4-L10-CH1-L11-Fc; and the third polypeptide has a structure represented by VL3-L12-VL4-L13-CL. In some aspects, the TAA bound by VL1 and VH1 is CD19 and the TAA bound by VL2 and VH2 is CD20. In some aspects, the TAA bound by VL1 and VH1 is CD20 and the TAA bound by VL2 and VH2 is CD19. In some aspects, the TAA bound by VL1 and VH1 is cMet and the TAA bound by VL2 and VH2 is Trop2. In some aspects, the TAA bound by VL1 and VH1 is Trop2 and the TAA bound by VL2 and VH2 is cMet.

In some aspects, the invention is directed to an antigen binding polypeptide complex comprising a first polypeptide, a second polypeptide, and a third polypeptide; wherein the first polypeptide has a structure represented by VL1-L1-VL2-L2-VH2-L3-VH1-L4-Fc; or VH1-L5-VH2-L6-VL2-L7-VL1-L8-Fc; wherein the second polypeptide has a structure represented by VH3-L9-VH4-CH1-Fc; wherein the third polypeptide has a structure represented by VL3-L10-VL4-CL; wherein VL1 is a first immunoglobulin light chain variable region that specifically binds to a TAA; VL2 is a second immunoglobulin light chain variable region that specifically binds to a TAA; VL3 is a third immunoglobulin light chain variable region that specifically binds to CD28; VL4 is a fourth immunoglobulin light chain variable region that specifically binds to human CD3; VH1 is a first immunoglobulin heavy chain variable region that specifically binds to a TAA; VH2 is a second immunoglobulin heavy chain variable region that specifically binds to a TAA; VH3 is a third immunoglobulin heavy chain variable region that specifically binds to CD28; VH4 is a fourth immunoglobulin heavy chain variable region that specifically binds to human CD3; Fc is a region comprising an immunoglobulin heavy chain constant region 2 (CH2), an immunoglobulin heavy chain constant region 3 (CH3), and optionally, an immunoglobulin hinge; CH1 is an immunoglobulin heavy chain constant region 1; CL is an immunoglobulin light chain constant region; and L1-L10 are amino acid linkers. In some aspects, the invention is directed to an antigen binding polypeptide complex comprising a first polypeptide, a second polypeptide, and a third polypeptide; wherein the first polypeptide has a structure represented by VL1-L1-VL2-L2-VH2-L3-VH1-L4-Fc; or VH1-L5-VL2-L6-VH2-L7-VL1-L8-Fc; wherein the second polypeptide has a structure represented by VH3-L9-VH4-CH1-Fc; wherein the third polypeptide has a structure represented by VL3-L10-VL4-CL; wherein VL1 is a first immunoglobulin light chain variable region that specifically binds to a TAA; VL2 is a second immunoglobulin light chain variable region that specifically binds to a TAA; VL3 is a third immunoglobulin light chain variable region that specifically binds to CD28; VL4 is a fourth immunoglobulin light chain variable region that specifically binds to human CD3; VH1 is a first immunoglobulin heavy chain variable region that specifically binds to a TAA; VH2 is a second immunoglobulin heavy chain variable region that specifically binds to a TAA; VH3 is a third immunoglobulin heavy chain variable region that specifically binds to CD28; VH4 is a fourth immunoglobulin heavy chain variable region that specifically binds to human CD3; Fc is a region comprising an immunoglobulin heavy chain constant region 2 (CH2), an immunoglobulin heavy chain constant region 3 (CH3), and optionally, an immunoglobulin hinge; CH1 is an immunoglobulin heavy chain constant region 1; CL is an immunoglobulin light chain constant region; and L1-L13 are amino acid linkers. In some aspects, the first polypeptide has a structure represented by VL1-L1-VL2-L2-VH2-L3-VH1-L4-Fc; the second polypeptide has a structure represented by VH3-L9-VH4-CH1-Fc; and the third polypeptide has a structure represented by VL3-L10-VL4-CL. In some aspects, the first polypeptide has a structure represented by VH1-L5-VL2-L6-VH2-L7-VL1-L8-Fc; the second polypeptide has a structure represented by VH3-L9-VH4-CH1-Fc; and the third polypeptide has a structure represented by VL3-L10-VL4-CL. In some aspects, the TAA bound by VL1 and VH1 is CD19 and the TAA bound by VL2 and VH2 is CD20. In some aspects, the TAA bound by VL1 and VH1 is CD20 and the TAA bound by VL2 and VH2 is CD19. In some aspects, the TAA bound by VL1 and VH1 is cMet and the TAA bound by VL2 and VH2 is Trop2. In some aspects, the TAA bound by VL1 and VH1 is Trop2 and the TAA bound by VL2 and VH2 is cMet.

In some aspects, the invention is directed to an antigen binding polypeptide complex comprising a first polypeptide, a second polypeptide, and a third polypeptide; wherein the first polypeptide has a structure represented by VL1-L1-VL2-L2-VH2-L3-VH1-L4-Fc; or VH1-L5-VH2-L6-VL2-L7-VL1-L8-Fc; wherein the second polypeptide has a structure represented by VH3-L9-VH4-L10-CH1-L11-Fc; wherein the third polypeptide has a structure represented by VL3-L12-VL4-L13-CL; wherein VL1 is a first immunoglobulin light chain variable region that specifically binds to an immune stimulating receptor; VL2 is a second immunoglobulin light chain variable region that specifically binds to a TAA; VL3 is a third immunoglobulin light chain variable region that specifically binds to a TAA; VL4 is a fourth immunoglobulin light chain variable region that specifically binds to human CD3; VH1 is a first immunoglobulin heavy chain variable region that specifically binds to an immune stimulating receptor; VH2 is a second immunoglobulin heavy chain variable region that specifically binds to a TAA; VH3 is a third immunoglobulin heavy chain variable region that specifically binds to a TAA; VH4 is a fourth immunoglobulin heavy chain variable region that specifically binds to human CD3; Fc is a region comprising an immunoglobulin heavy chain constant region 2 (CH2), an immunoglobulin heavy chain constant region 3 (CH3), and optionally, an immunoglobulin hinge; CH1 is an immunoglobulin heavy chain constant region 1; CL is an immunoglobulin light chain constant region; and L1-L13 are amino acid linkers. In some aspects, the invention is directed to an antigen binding polypeptide complex comprising a first polypeptide, a second polypeptide, and a third polypeptide; wherein the first polypeptide has a structure represented by VL1-L1-VL2-L2-VH2-L3-VH1-L4-Fc; or VH1-L5-VL2-L6-VH2-L7-VL1-L8-Fc; wherein the second polypeptide has a structure represented by VH3-L9-VH4-L10-CH1-L11-Fc; wherein the third polypeptide has a structure represented by VL3-L12-VL4-L13-CL; wherein VL1 is a first immunoglobulin light chain variable region that specifically binds to an immune stimulating receptor; VL2 is a second immunoglobulin light chain variable region that specifically binds to a TAA; VL3 is a third immunoglobulin light chain variable region that specifically binds to a TAA; VL4 is a fourth immunoglobulin light chain variable region that specifically binds to human CD3; VH1 is a first immunoglobulin heavy chain variable region that specifically binds to an immune stimulating receptor; VH2 is a second immunoglobulin heavy chain variable region that specifically binds to a TAA; VH3 is a third immunoglobulin heavy chain variable region that specifically binds to a TAA; VH4 is a fourth immunoglobulin heavy chain variable region that specifically binds to human CD3; Fc is a region comprising an immunoglobulin heavy chain constant region 2 (CH2), an immunoglobulin heavy chain constant region 3 (CH3), and optionally, an immunoglobulin hinge; CH1 is an immunoglobulin heavy chain constant region 1; CL is an immunoglobulin light chain constant region; and L1-L13 are amino acid linkers. In some aspects, the first polypeptide has a structure represented by VL1-L1-VL2-L2-VH2-L3-VH1-L4-Fc; the second polypeptide has a structure represented by VH3-L9-VH4-L10-CH1-L11-Fc; and the third polypeptide has a structure represented by VL3-L12-VL4-L13-CL. In some aspects, the first polypeptide has a structure represented by VH1-L5-VL2-L6-VH2-L7-VL1-L8-Fc; the second polypeptide has a structure represented by VH3-L9-VH4-L10-CH1-L11-Fc; and the third polypeptide has a structure represented by VL3-L12-VL4-L13-CL. In some aspects, the immune stimulating receptor bound by VL1 and VH1 is CD28, the TAA bound by VL2 and VH2 is CD19 and/or the TAA bound by VL3 and VH3 is CD20. In some aspects, the immune stimulating receptor bound by VL1 and VH1 is CD28, the TAA bound by VL2 and VH2 is CD20 and/or the TAA bound by VL3 and VH3 is CD19. In some aspects, the immune stimulating receptor bound by VL1 and VH1 is CD28, the TAA bound by VL2 and VH2 is cMet and/or the TAA bound by VL3 and VH3 is Trop2. In some aspects, the immune stimulating receptor bound by VL1 and VH1 is CD28, the TAA bound by VL2 and VH2 is Trop2 and/or the TAA bound by VL3 and VH3 is cMet.

In some other aspects, the invention is directed to an antigen binding polypeptide complex comprising a first polypeptide, a second polypeptide, and a third polypeptide; wherein the first polypeptide has a structure represented by VL1-L1-VL2-L2-VH2-L3-VH1-L4-Fc; or VH1-L5-VH2-L6-VL2-L7-VL1-L8-Fc; wherein the second polypeptide has a structure represented by VH3-L9-VH4-CH1-Fc; wherein the third polypeptide has a structure represented by VL3-L10-VL4-CL; wherein VL1 is a first immunoglobulin light chain variable region that specifically binds to an immune stimulating receptor; VL2 is a second immunoglobulin light chain variable region that specifically binds to a TAA; VL3 is a third immunoglobulin light chain variable region that specifically binds to a TAA; VL4 is a fourth immunoglobulin light chain variable region that specifically binds to human CD3; VH1 is a first immunoglobulin heavy chain variable region that specifically binds to an immune stimulating receptor; VH2 is a second immunoglobulin heavy chain variable region that specifically binds to a TAA; VH3 is a third immunoglobulin heavy chain variable region that specifically binds to a TAA; VH4 is a fourth immunoglobulin heavy chain variable region that specifically binds to human CD3; Fc is a region comprising an immunoglobulin heavy chain constant region 2 (CH2), an immunoglobulin heavy chain constant region 3 (CH3), and optionally, an immunoglobulin hinge; CH1 is an immunoglobulin heavy chain constant region 1; CL is an immunoglobulin light chain constant region; and L1-L13 are amino acid linkers. In some aspects, the invention is directed to an antigen binding polypeptide complex comprising a first polypeptide, a second polypeptide, and a third polypeptide; wherein the first polypeptide has a structure represented by VL1-L1-VL2-L2-VH2-L3-VH1-L4-Fc; or VH1-L5-VL2-L6-VH2-L7-VL1-L8-Fc; wherein the second polypeptide has a structure represented by VH3-L9-VH4-CH1-Fc; wherein the third polypeptide has a structure represented by VL3-L10-VL4-CL; wherein VL1 is a first immunoglobulin light chain variable region that specifically binds to an immune stimulating receptor; VL2 is a second immunoglobulin light chain variable region that specifically binds to a TAA; VL3 is a third immunoglobulin light chain variable region that specifically binds to a TAA; VL4 is a fourth immunoglobulin light chain variable region that specifically binds to human CD3; VH1 is a first immunoglobulin heavy chain variable region that specifically binds to an immune stimulating receptor; VH2 is a second immunoglobulin heavy chain variable region that specifically binds to a TAA; VH3 is a third immunoglobulin heavy chain variable region that specifically binds to a TAA; VH4 is a fourth immunoglobulin heavy chain variable region that specifically binds to human CD3; Fc is a region comprising an immunoglobulin heavy chain constant region 2 (CH2), an immunoglobulin heavy chain constant region 3 (CH3), and optionally, an immunoglobulin hinge; CH1 is an immunoglobulin heavy chain constant region 1; CL is an immunoglobulin light chain constant region; and L1-L10 are amino acid linkers. In some aspects, the first polypeptide has a structure represented by VL1-L1-VL2-L2-VH2-L3-VH1-L4-Fc; the second polypeptide has a structure represented by VH3-L9-VH4-CH1-Fc; and the third polypeptide has a structure represented by VL3-L10-VL4-CL. In some aspects, the first polypeptide has a structure represented by VH1-L5-VL2-L6-VH2-L7-VL1-L8-Fc; the second polypeptide has a structure represented by VH3-L9-VH4-CH1-Fc; and the third polypeptide has a structure represented by VL3-L10-VL4-CL. In some aspects, the immune stimulating receptor bound by VL1 and VH1 is CD28, the TAA bound by VL2 and VH2 is CD19 and/or the TAA bound by VL3 and VH3 is CD20. In some aspects, the immune stimulating receptor bound by VL1 and VH1 is CD28, the TAA bound by VL2 and VH2 is CD20 and/or the TAA bound by VL3 and VH3 is CD19. In some aspects, the immune stimulating receptor bound by VL1 and VH1 is CD28, the TAA bound by VL2 and VH2 is cMet and/or the TAA bound by VL3 and VH3 is Trop2. In some aspects, the immune stimulating receptor bound by VL1 and VH1 is CD28, the TAA bound by VL2 and VH2 is Trop2 and/or the TAA bound by VL3 and VH3 is cMet.

In some aspects, the invention is directed to an antigen binding polypeptide complex comprising a first polypeptide, a second polypeptide, and a third polypeptide; wherein the first polypeptide has a structure represented by VL1-L1-VL2-L2-VH2-L3-VH1-L4-Fc; or VH1-L5-VH2-L6-VL2-L7-VL1-L8-Fc; wherein the second polypeptide has a structure represented by VH3-L9-VH4-L10-CH1-L11-Fc; wherein the third polypeptide has a structure represented by VL3-L12-VL4-L13-CL; wherein VL1 is a first immunoglobulin light chain variable region that specifically binds to CD28; VL2 is a second immunoglobulin light chain variable region that specifically binds to a TAA; VL3 is a third immunoglobulin light chain variable region that specifically binds to a TAA; VL4 is a fourth immunoglobulin light chain variable region that specifically binds to human CD3; VH1 is a first immunoglobulin heavy chain variable region that specifically binds to CD28; VH2 is a second immunoglobulin heavy chain variable region that specifically binds to a TAA; VH3 is a third immunoglobulin heavy chain variable region that specifically binds to a TAA; VH4 is a fourth immunoglobulin heavy chain variable region that specifically binds to human CD3; Fc is a region comprising an immunoglobulin heavy chain constant region 2 (CH2), an immunoglobulin heavy chain constant region 3 (CH3), and optionally, an immunoglobulin hinge; CH1 is an immunoglobulin heavy chain constant region 1; CL is an immunoglobulin light chain constant region; and L1-L13 are amino acid linkers. In some aspects, the invention is directed to an antigen binding polypeptide complex comprising a first polypeptide, a second polypeptide, and a third polypeptide; wherein the first polypeptide has a structure represented by VL1-L1-VL2-L2-VH2-L3-VH1-L4-Fc; or VH1-L5-VL2-L6-VH2-L7-VL1-L8-Fc; wherein the second polypeptide has a structure represented by VH3-L9-VH4-L10-CH1-L11-Fc; wherein the third polypeptide has a structure represented by VL3-L12-VL4-L13-CL; wherein VL1 is a first immunoglobulin light chain variable region that specifically binds to CD28; VL2 is a second immunoglobulin light chain variable region that specifically binds to a TAA; VL3 is a third immunoglobulin light chain variable region that specifically binds to a TAA; VL4 is a fourth immunoglobulin light chain variable region that specifically binds to human CD3; VH1 is a first immunoglobulin heavy chain variable region that specifically binds to CD28; VH2 is a second immunoglobulin heavy chain variable region that specifically binds to a TAA; VH3 is a third immunoglobulin heavy chain variable region that specifically binds to a TAA; VH4 is a fourth immunoglobulin heavy chain variable region that specifically binds to human CD3; Fc is a region comprising an immunoglobulin heavy chain constant region 2 (CH2), an immunoglobulin heavy chain constant region 3 (CH3), and optionally, an immunoglobulin hinge; CH1 is an immunoglobulin heavy chain constant region 1; CL is an immunoglobulin light chain constant region; and L1-L13 are amino acid linkers. In some aspects, the first polypeptide has a structure represented by VL1-L1-VL2-L2-VH2-L3-VH1-L4-Fc; the second polypeptide has a structure represented by VH3-L9-VH4-L10-CH1-L11-Fc; and the third polypeptide has a structure represented by VL3-L12-VL4-L13-CL. In some aspects, the first polypeptide has a structure represented by VH1-L5-VL2-L6-VH2-L7-VL1-L8-Fc; the second polypeptide has a structure represented by VH3-L9-VH4-L10-CH1-L11-Fc; and the third polypeptide has a structure represented by VL3-L12-VL4-L13-CL. In some aspects, the TAA bound by VL2 and VH2 is CD19 and the TAA bound by VL3 and VH3 is CD20. In some aspects, the TAA bound by VL2 and VH2 is CD20 and the TAA bound by VL3 and VH3 is CD19. In some aspects, the TAA bound by VL2 and VH2 is cMet and the TAA bound by VL3 and VH3 is Trop2. In some aspects, the TAA bound by VL2 and VH2 is Trop2 and the TAA bound by VL3 and VH3 is cMet.

In other some aspects, the invention is directed to an antigen binding polypeptide complex comprising a first polypeptide, a second polypeptide, and a third polypeptide; wherein the first polypeptide has a structure represented by VL1-L1-VL2-L2-VH2-L3-VH1-L4-Fc; or VH1-L5-VH2-L6-VL2-L7-VL1-L8-Fc; wherein the second polypeptide has a structure represented by VH3-L9-VH4-CH1-Fc; wherein the third polypeptide has a structure represented by VL3-L10-VL4-CL; wherein VL1 is a first immunoglobulin light chain variable region that specifically binds to CD28; VL2 is a second immunoglobulin light chain variable region that specifically binds to a TAA; VL3 is a third immunoglobulin light chain variable region that specifically binds to a TAA; VL4 is a fourth immunoglobulin light chain variable region that specifically binds to human CD3; VH1 is a first immunoglobulin heavy chain variable region that specifically binds to CD28; VH2 is a second immunoglobulin heavy chain variable region that specifically binds to a TAA; VH3 is a third immunoglobulin heavy chain variable region that specifically binds to a TAA; VH4 is a fourth immunoglobulin heavy chain variable region that specifically binds to human CD3; Fc is a region comprising an immunoglobulin heavy chain constant region 2 (CH2), an immunoglobulin heavy chain constant region 3 (CH3), and optionally, an immunoglobulin hinge; CH1 is an immunoglobulin heavy chain constant region 1; CL is an immunoglobulin light chain constant region; and L1-L13 are amino acid linkers. In some aspects, the invention is directed to an antigen binding polypeptide complex comprising a first polypeptide, a second polypeptide, and a third polypeptide; wherein the first polypeptide has a structure represented by VL1-L1-VL2-L2-VH2-L3-VH1-L4-Fc; or VH1-L5-VL2-L6-VH2-L7-VL1-L8-Fc; wherein the second polypeptide has a structure represented by VH3-L9-VH4-CH1-Fc; wherein the third polypeptide has a structure represented by VL3-L10-VL4-CL; wherein VL1 is a first immunoglobulin light chain variable region that specifically binds to CD28; VL2 is a second immunoglobulin light chain variable region that specifically binds to a TAA; VL3 is a third immunoglobulin light chain variable region that specifically binds to a TAA; VL4 is a fourth immunoglobulin light chain variable region that specifically binds to human CD3; VH1 is a first immunoglobulin heavy chain variable region that specifically binds to CD28; VH2 is a second immunoglobulin heavy chain variable region that specifically binds to a TAA; VH3 is a third immunoglobulin heavy chain variable region that specifically binds to a TAA; VH4 is a fourth immunoglobulin heavy chain variable region that specifically binds to human CD3; Fc is a region comprising an immunoglobulin heavy chain constant region 2 (CH2), an immunoglobulin heavy chain constant region 3 (CH3), and optionally, an immunoglobulin hinge; CH1 is an immunoglobulin heavy chain constant region 1; CL is an immunoglobulin light chain constant region; and L1-L13 are amino acid linkers. In some aspects, the first polypeptide has a structure represented by VL1-L1-VL2-L2-VH2-L3-VH1-L4-Fc; the second polypeptide has a structure represented by VH3-L9-VH4-CH1-Fc; and the third polypeptide has a structure represented by VL3-L10-VL4-CL. In some aspects, the first polypeptide has a structure represented by VH1-L5-VL2-L6-VH2-L7-VL1-L8-Fc; the second polypeptide has a structure represented by VH3-L9-VH4-CH1-Fc; and the third polypeptide has a structure represented by VL3-L1-VL4-L13-CL. In some aspects, the TAA bound by VL2 and VH2 is CD19 and the TAA bound by VL3 and VH3 is CD20. In some aspects, the TAA bound by VL2 and VH2 is CD20 and the TAA bound by VL3 and VH3 is CD19. In some aspects, the TAA bound by VL2 and VH2 is cMet and the TAA bound by VL3 and VH3 is Trop2. In some aspects, the TAA bound by VL2 and VH2 is Trop2 and the TAA bound by VL3 and VH3 is cMet.

In some aspects, the invention is directed to an antigen binding polypeptide complex comprising a first polypeptide, a second polypeptide, and a third polypeptide; wherein the first polypeptide has a structure represented by VL1-L1-VL2-L2-VH2-L3-VH1-L4-Fc; or VH1-L5-VH2-L6-VL2-L7-VL1-L8-Fc; wherein the second polypeptide has a structure represented by VH3-L9-VH4-L10-CH1-L11-Fc; wherein the third polypeptide has a structure represented by VL3-L12-VL4-L13-CL; wherein VL1 is a first immunoglobulin light chain variable region that specifically binds to a TAA; VL2 is a second immunoglobulin light chain variable region that specifically binds to an immune stimulating receptor; VL3 is a third immunoglobulin light chain variable region that specifically binds to a TAA; VL4 is a fourth immunoglobulin light chain variable region that specifically binds to human CD3; VH1 is a first immunoglobulin heavy chain variable region that specifically binds to a TAA; VH2 is a second immunoglobulin heavy chain variable region that specifically binds to an immune stimulating receptor; VH3 is a third immunoglobulin heavy chain variable region that specifically binds to a TAA; VH4 is a fourth immunoglobulin heavy chain variable region that specifically binds to human CD3; Fc is a region comprising an immunoglobulin heavy chain constant region 2 (CH2), an immunoglobulin heavy chain constant region 3 (CH3), and optionally, an immunoglobulin hinge; CH1 is an immunoglobulin heavy chain constant region 1; CL is an immunoglobulin light chain constant region; and L1-L13 are amino acid linkers. In some aspects, the invention is directed to an antigen binding polypeptide complex comprising a first polypeptide, a second polypeptide, and a third polypeptide; wherein the first polypeptide has a structure represented by VL1-L1-VL2-L2-VH2-L3-VH1-L4-Fc; or VH1-L5-VL2-L6-VH2-L7-VL1-L8-Fc; wherein the second polypeptide has a structure represented by VH3-L9-VH4-L10-CH1-L11-Fc; wherein the third polypeptide has a structure represented by VL3-L12-VL4-L13-CL; wherein VL1 is a first immunoglobulin light chain variable region that specifically binds to a TAA; VL2 is a second immunoglobulin light chain variable region that specifically binds to an immune stimulating receptor; VL3 is a third immunoglobulin light chain variable region that specifically binds to a TAA; VL4 is a fourth immunoglobulin light chain variable region that specifically binds to human CD3; VH1 is a first immunoglobulin heavy chain variable region that specifically binds to a TAA; VH2 is a second immunoglobulin heavy chain variable region that specifically binds to an immune stimulating receptor; VH3 is a third immunoglobulin heavy chain variable region that specifically binds to a TAA; VH4 is a fourth immunoglobulin heavy chain variable region that specifically binds to human CD3; Fc is a region comprising an immunoglobulin heavy chain constant region 2 (CH2), an immunoglobulin heavy chain constant region 3 (CH3), and optionally, an immunoglobulin hinge; CH1 is an immunoglobulin heavy chain constant region 1; CL is an immunoglobulin light chain constant region; and L1-L13 are amino acid linkers. In some aspects, the first polypeptide has a structure represented by VL1-L1-VL2-L2-VH2-L3-VH1-L4-Fc; the second polypeptide has a structure represented by VH3-L9-VH4-L10-CH1-L11-Fc; and the third polypeptide has a structure represented by VL3-L12-VL4-L13-CL. In some aspects, the first polypeptide has a structure represented by VH1-L5-VL2-L6-VH2-L7-VL1-L8-Fc; the second polypeptide has a structure represented by VH3-L9-VH4-L10-CH1-L11-Fc; and the third polypeptide has a structure represented by VL3-L12-VL4-L13-CL. In some aspects, the TAA bound by VL1 and VH1 is CD19, the immune stimulating receptor bound by VL2 and VH2 is CD28 and/or the TAA bound by VL3 and VH3 is CD20. In some aspects, the TAA bound by VL1 and VH1 is CD20, the immune stimulating receptor bound by VL2 and VH2 is CD28 and/or the TAA bound by VL3 and VH3 is CD19. In some aspects, the TAA bound by VL1 and VH1 is cMet, the immune stimulating receptor bound by VL2 and VH2 is CD28 and/or the TAA bound by VL3 and VH3 is Trop2. In some aspects, the TAA bound by VL1 and VH1 is Trop2, the immune stimulating receptor bound by VL2 and VH2 is CD28 and/or the TAA bound by VL3 and VH3 is cMet. In some aspects, the the immune stimulating receptor bound by VL2 and VH2 is CD28.

In some aspects, the invention is directed to an antigen binding polypeptide complex comprising a first polypeptide, a second polypeptide, and a third polypeptide; wherein the first polypeptide has a structure represented by VL1-L1-VL2-L2-VH2-L3-VH1-L4-Fc; or VH1-L5-VH2-L6-VL2-L7-VL1-L8-Fc; wherein the second polypeptide has a structure represented by VH3-L9-VH4-CH1-Fc; wherein the third polypeptide has a structure represented by VL3-L10-VL4-CL; wherein VL1 is a first immunoglobulin light chain variable region that specifically binds to a TAA; VL2 is a second immunoglobulin light chain variable region that specifically binds to an immune stimulating receptor; VL3 is a third immunoglobulin light chain variable region that specifically binds to a TAA; VL4 is a fourth immunoglobulin light chain variable region that specifically binds to human CD3; VH1 is a first immunoglobulin heavy chain variable region that specifically binds to a TAA; VH2 is a second immunoglobulin heavy chain variable region that specifically binds to an immune stimulating receptor; VH3 is a third immunoglobulin heavy chain variable region that specifically binds to a TAA; VH4 is a fourth immunoglobulin heavy chain variable region that specifically binds to human CD3; Fc is a region comprising an immunoglobulin heavy chain constant region 2 (CH2), an immunoglobulin heavy chain constant region 3 (CH3), and optionally, an immunoglobulin hinge; CH1 is an immunoglobulin heavy chain constant region 1; CL is an immunoglobulin light chain constant region; and L1-L10 are amino acid linkers. In some aspects, the invention is directed to an antigen binding polypeptide complex comprising a first polypeptide, a second polypeptide, and a third polypeptide; wherein the first polypeptide has a structure represented by VL1-L1-VL2-L2-VH2-L3-VH1-L4-Fc; or VH1-L5-VL2-L6-VH2-L7-VL1-L8-Fc; wherein the second polypeptide has a structure represented by VH3-L9-VH4-CH1-Fc; wherein the third polypeptide has a structure represented by VL3-L10-VL4-CL; wherein VL1 is a first immunoglobulin light chain variable region that specifically binds to a TAA; VL2 is a second immunoglobulin light chain variable region that specifically binds to an immune stimulating receptor; VL3 is a third immunoglobulin light chain variable region that specifically binds to a TAA; VL4 is a fourth immunoglobulin light chain variable region that specifically binds to human CD3; VH1 is a first immunoglobulin heavy chain variable region that specifically binds to a TAA; VH2 is a second immunoglobulin heavy chain variable region that specifically binds to an immune stimulating receptor; VH3 is a third immunoglobulin heavy chain variable region that specifically binds to a TAA; VH4 is a fourth immunoglobulin heavy chain variable region that specifically binds to human CD3; Fc is a region comprising an immunoglobulin heavy chain constant region 2 (CH2), an immunoglobulin heavy chain constant region 3 (CH3), and optionally, an immunoglobulin hinge; CH1 is an immunoglobulin heavy chain constant region 1; CL is an immunoglobulin light chain constant region; and L1-L10 are amino acid linkers. In some aspects, the first polypeptide has a structure represented by VL1-L1-VL2-L2-VH2-L3-VH1-L4-Fc; the second polypeptide has a structure represented by VH3-L9-VH4-CH1-Fc; and the third polypeptide has a structure represented by VL3-L10-VL4-CL. In some aspects, the first polypeptide has a structure represented by VH1-L5-VL2-L6-VH2-L7-VL1-L8-Fc; the second polypeptide has a structure represented by VH3-L9-VH4-CH1-Fc; and the third polypeptide has a structure represented by VL3-L10-VL4-CL. In some aspects, the TAA bound by VL1 and VH1 is CD19, the immune stimulating receptor bound by VL2 and VH2 is CD28 and/or the TAA bound by VL3 and VH3 is CD20. In some aspects, the TAA bound by VL1 and VH1 is CD20, the immune stimulating receptor bound by VL2 and VH2 is CD28 and/or the TAA bound by VL3 and VH3 is CD19. In some aspects, the TAA bound by VL1 and VH1 is cMet, the immune stimulating receptor bound by VL2 and VH2 is CD28 and/or the TAA bound by VL3 and VH3 is Trop2. In some aspects, the TAA bound by VL1 and VH1 is Trop2, the immune stimulating receptor bound by VL2 and VH2 is CD28 and/or the TAA bound by VL3 and VH3 is cMet. In some aspects, the the immune stimulating receptor bound by VL2 and VH2 is CD28.

In some aspects, the invention is directed to an antigen binding polypeptide complex comprising a first polypeptide, a second polypeptide, and a third polypeptide; wherein the first polypeptide has a structure represented by VL1-L1-VL2-L2-VH2-L3-VH1-L4-Fc; or VH1-L5-VH2-L6-VL2-L7-VL1-L8-Fc; wherein the second polypeptide has a structure represented by VH3-L9-VH4-L10-CH1-L11-Fc; wherein the third polypeptide has a structure represented by VL3-L12-VL4-L13-CL; wherein VL1 is a first immunoglobulin light chain variable region that specifically binds to a TAA; VL2 is a second immunoglobulin light chain variable region that specifically binds to CD28; VL3 is a third immunoglobulin light chain variable region that specifically binds to a TAA; VL4 is a fourth immunoglobulin light chain variable region that specifically binds to human CD3; VH1 is a first immunoglobulin heavy chain variable region that specifically binds to a TAA; VH2 is a second immunoglobulin heavy chain variable region that specifically binds to CD28; VH3 is a third immunoglobulin heavy chain variable region that specifically binds to a TAA; VH4 is a fourth immunoglobulin heavy chain variable region that specifically binds to human CD3; Fc is a region comprising an immunoglobulin heavy chain constant region 2 (CH2), an immunoglobulin heavy chain constant region 3 (CH3), and optionally, an immunoglobulin hinge; CH1 is an immunoglobulin heavy chain constant region 1; CL is an immunoglobulin light chain constant region; and L1-L13 are amino acid linkers. In some aspects, the invention is directed to an antigen binding polypeptide complex comprising a first polypeptide, a second polypeptide, and a third polypeptide; wherein the first polypeptide has a structure represented by VL1-L1-VL2-L2-VH2-L3-VH1-L4-Fc; or VH1-L5-VL2-L6-VH2-L7-VL1-L8-Fc; wherein the second polypeptide has a structure represented by VH3-L9-VH4-L10-CH1-L11-Fc; wherein the third polypeptide has a structure represented by VL3-L12-VL4-L13-CL; wherein VL1 is a first immunoglobulin light chain variable region that specifically binds to a TAA; VL2 is a second immunoglobulin light chain variable region that specifically binds to CD28; VL3 is a third immunoglobulin light chain variable region that specifically binds to a TAA; VL4 is a fourth immunoglobulin light chain variable region that specifically binds to human CD3; VH1 is a first immunoglobulin heavy chain variable region that specifically binds to a TAA; VH2 is a second immunoglobulin heavy chain variable region that specifically binds to CD28; VH3 is a third immunoglobulin heavy chain variable region that specifically binds to a TAA; VH4 is a fourth immunoglobulin heavy chain variable region that specifically binds to human CD3; Fc is a region comprising an immunoglobulin heavy chain constant region 2 (CH2), an immunoglobulin heavy chain constant region 3 (CH3), and optionally, an immunoglobulin hinge; CH1 is an immunoglobulin heavy chain constant region 1; CL is an immunoglobulin light chain constant region; and L1-L13 are amino acid linkers. In some aspects, the first polypeptide has a structure represented by VL1-L1-VL2-L2-VH2-L3-VH1-L4-Fc; the second polypeptide has a structure represented by VH3-L9-VH4-L10-CH1-L11-Fc; and the third polypeptide has a structure represented by VL3-L12-VL4-L13-CL. In some aspects, the first polypeptide has a structure represented by VH1-L5-VL2-L6-VH2-L7-VL1-L8-Fc; the second polypeptide has a structure represented by VH3-L9-VH4-L10-CH1-L11-Fc; and the third polypeptide has a structure represented by VL3-L12-VL4-L13-CL. In some aspects, the TAA bound by VL1 and VH1 is CD19 and the TAA bound by VL3 and VH3 is CD20. In some aspects, the TAA bound by VL1 and VH1 is CD20 and the TAA bound by VL3 and VH3 is CD19. In some aspects, the TAA bound by VL1 and VH1 is cMet and the TAA bound by VL3 and VH3 is Trop2. In some aspects, the TAA bound by VL1 and VH1 is Trop2 and the TAA bound by VL3 and VH3 is cMet.

In some aspects, the invention is directed to an antigen binding polypeptide complex comprising a first polypeptide, a second polypeptide, and a third polypeptide; wherein the first polypeptide has a structure represented by VL1-L1-VL2-L2-VH2-L3-VH1-L4-Fc; or VH1-L5-VH2-L6-VL2-L7-VL1-L8-Fc; wherein the second polypeptide has a structure represented by VH3-L9-VH4-CH1-Fc; wherein the third polypeptide has a structure represented by VL3-L10-VL4-CL; wherein VL1 is a first immunoglobulin light chain variable region that specifically binds to a TAA; VL2 is a second immunoglobulin light chain variable region that specifically binds to CD28; VL3 is a third immunoglobulin light chain variable region that specifically binds to a TAA; VL4 is a fourth immunoglobulin light chain variable region that specifically binds to human CD3; VH1 is a first immunoglobulin heavy chain variable region that specifically binds to a TAA; VH2 is a second immunoglobulin heavy chain variable region that specifically binds to CD28; VH3 is a third immunoglobulin heavy chain variable region that specifically binds to a TAA; VH4 is a fourth immunoglobulin heavy chain variable region that specifically binds to human CD3; Fc is a region comprising an immunoglobulin heavy chain constant region 2 (CH2), an immunoglobulin heavy chain constant region 3 (CH3), and optionally, an immunoglobulin hinge; CH1 is an immunoglobulin heavy chain constant region 1; CL is an immunoglobulin light chain constant region; and L1-L10 are amino acid linkers. In some aspects, the invention is directed to an antigen binding polypeptide complex comprising a first polypeptide, a second polypeptide, and a third polypeptide; wherein the first polypeptide has a structure represented by VL1-L1-VL2-L2-VH2-L3-VH1-L4-Fc; or VH1-L5-VL2-L6-VH2-L7-VL1-L8-Fc; wherein the second polypeptide has a structure represented by VH3-L9-VH4-CH1-Fc; wherein the third polypeptide has a structure represented by VL3-L10-VL4-CL; wherein VL1 is a first immunoglobulin light chain variable region that specifically binds to a TAA; VL2 is a second immunoglobulin light chain variable region that specifically binds to CD28; VL3 is a third immunoglobulin light chain variable region that specifically binds to a TAA; VL4 is a fourth immunoglobulin light chain variable region that specifically binds to human CD3; VH1 is a first immunoglobulin heavy chain variable region that specifically binds to a TAA; VH2 is a second immunoglobulin heavy chain variable region that specifically binds to CD28; VH3 is a third immunoglobulin heavy chain variable region that specifically binds to a TAA; VH4 is a fourth immunoglobulin heavy chain variable region that specifically binds to human CD3; Fc is a region comprising an immunoglobulin heavy chain constant region 2 (CH2), an immunoglobulin heavy chain constant region 3 (CH3), and optionally, an immunoglobulin hinge; CH1 is an immunoglobulin heavy chain constant region 1; CL is an immunoglobulin light chain constant region; and L1-L13 are amino acid linkers. In some aspects, the first polypeptide has a structure represented by VL1-L1-VL2-L2-VH2-L3-VH1-L4-Fc; the second polypeptide has a structure represented by VH3-L9-VH4-CH1-Fc; and the third polypeptide has a structure represented by VL3-L10-VL4-CL. In some aspects, the first polypeptide has a structure represented by VH1-L5-VL2-L6-VH2-L7-VL1-L8-Fc; the second polypeptide has a structure represented by VH3-L9-VH4-CH1-Fc; and the third polypeptide has a structure represented by VL3-L10-VL4-CL. In some aspects, the TAA bound by VL1 and VH1 is CD19 and the TAA bound by VL3 and VH3 is CD20. In some aspects, the TAA bound by VL1 and VH1 is CD20 and the TAA bound by VL3 and VH3 is CD19. In some aspects, the TAA bound by VL1 and VH1 is cMet and the TAA bound by VL3 and VH3 is Trop2. In some aspects, the TAA bound by VL1 and VH1 is Trop2 and the TAA bound by VL3 and VH3 is cMet.

In some aspects, the invention is directed to an antigen binding polypeptide complex comprising a first polypeptide and a second polypeptide; wherein the first polypeptide has a structure represented by VL1-L1-VL2-L2-VH2-L3-VH1-L4-Fc; or VH1-L5-VH2-L6-VL2-L7-VL1-L8-Fc; wherein the second polypeptide has a structure represented by VL3-L9-VL4-L10-VH4-L11-VH3-L12-Fc; or VH3-L13-VH4-L14-VL4-L15-VL3-L16-Fc; wherein VL1 is a first immunoglobulin light chain variable region that specifically binds to A2AR, APRIL, ATPDase, BAFF, BAFFR, BCMA, BlyS, BTK, BTLA, B7DC, B7H1, B7H2, B7H3, B7H4, B7H5, B7H6, B7H7, B7RP1, B7-4, C3, C5, CCL2, CCL3, CCL4, CCL5, CCL7, CCL8, CCL11, CCL15, CCL17, CCL19, CCL20, CCL21, CCL25, CCR3, CCR4, CD16A, CD19, CD20, CD24, CD27, CD28, CD30, CD38, CD39, CD40, CD40L, CD47, CD52, CD70, CD80, CD86, CD123, CD133, CD137, CD137L, CD160, CD272, CEACAM5, CLEC9, CLEC91, CRTH2, CSF-1, CSF-2, CSF-3, CXCL1, CXCL2, CXCL4, CXCL12, CXCL13, CXCR3, cMet, CTLA4, DLL3, DLL4, DNGR-1, E-cadherin, EGFR, ENTPD1, EpCAM, FCER1, FCER1A, FCER2, FGFR, FLAP, FOLH1, Gi24, GITR, GITRL, GPR5, GP100, GPRC5D, HER2, HER3, ICOSL, ICOS, HHLA2, HMGB1, HVEM, IDO, IFNa, IgE, IGF1R, IL2Rbeta, IL1, ILIA, IL1B, IL1F10, IL2, IL4, IL4Ra, IL5, IL5R, IL6, IL7, IL7Ra, IL8, IL9, IL9R, IL10, rhILlO, IL12, IL13, IL13Ra1, IL13Ra2, IL15, IL17, IL17Rb, IL18, IL22, IL23, IL25, IL7, IL33, IL35, ITGB4, ITK, KIR, LAG3, LAMP1, leptin, LPFS2, MHC class II, MUC-1, MUC-16, NCR3LG1, NKG2D, NKp46, NTPDase-1, OX40, OX40L, PD-1, PD-L1, PD-L2, PROM1, S152, SIRPalpha, SISP1, SLC, SPG64, ST2, STEAP1, STEAP2, Syk kinase, STEAP1, TROP2, TACI, TDO, TGFBETA, T14, TIGIT, TIM3, TLR, TLR2, TLR4, TLR5, TLR9, TMEF1, TNFa, TNFRSF7, Tp55, TREM1, TSLP, TSLPR, TWEAK, VEGF, VISTA, Vstm3, or WUCAM; VL2 is a second immunoglobulin light chain variable region that specifically binds to A2AR, APRIL, ATPDase, BAFF, BAFFR, BCMA, BlyS, BTK, BTLA, B7DC, B7H1, B7H2, B7H3, B7H4, B7H5, B7H6, B7H7, B7RP1, B7-4, C3, C5, CCL2, CCL3, CCL4, CCL5, CCL7, CCL8, CCL11, CCL15, CCL17, CCL19, CCL20, CCL21, CCL25, CCR3, CCR4, CD16A, CD19, CD20, CD24, CD27, CD28, CD30, CD38, CD39, CD40, CD40L, CD47, CD52, CD70, CD80, CD86, CD123, CD133, CD137, CD137L, CD160, CD272, CEACAM5, CLEC9, CLEC91, CRTH2, CSF-1, CSF-2, CSF-3, CXCL1, CXCL2, CXCL4, CXCL12, CXCL13, CXCR3, cMet, CTLA4, DLL3, DLL4, DNGR-1, E-cadherin, EGFR, ENTPD1, EpCAM, FCER1, FCER1A, FCER2, FGFR, FLAP, FOLH1, Gi24, GITR, GITRL, GPR5, GP100, GPRC5D, HER2, HER3, ICOSL, ICOS, HHLA2, HMGB1, HVEM, IDO, IFNa, IgE, IGF1R, IL2Rbeta, IL1, ILIA, IL1B, IL1F10, IL2, IL4, IL4Ra, IL5, IL5R, IL6, IL7, IL7Ra, IL8, IL9, IL9R, IL10, rhILlO, IL12, IL13, IL13Ra1, IL13Ra2, IL15, IL17, IL17Rb, IL18, IL22, IL23, IL25, IL7, IL33, IL35, ITGB4, ITK, KIR, LAG3, LAMP1, leptin, LPFS2, MHC class II, MUC-1, MUC-16, NCR3LG1, NKG2D, NKp46, NTPDase-1, OX40, OX40L, PD-1, PD-L1, PD-L2, PROM1, S152, SIRPalpha, SISP1, SLC, SPG64, ST2, STEAP1, STEAP2, Syk kinase, STEAP1, TROP2, TACI, TDO, TGFBETA, T14, TIGIT, TIM3, TLR, TLR2, TLR4, TLR5, TLR9, TMEF1, TNFa, TNFRSF7, Tp55, TREM1, TSLP, TSLPR, TWEAK, VEGF, VISTA, Vstm3, or WUCAM; VL3 is a third immunoglobulin light chain variable region that specifically binds to CD3; VL4 is a fourth immunoglobulin light chain variable region that specifically binds to A2AR, APRIL, ATPDase, BAFF, BAFFR, BCMA, BlyS, BTK, BTLA, B7DC, B7H1, B7H2, B7H3, B7H4, B7H5, B7H6, B7H7, B7RP1, B7-4, C3, C5, CCL2, CCL3, CCL4, CCL5, CCL7, CCL8, CCL11, CCL15, CCL17, CCL19, CCL20, CCL21, CCL25, CCR3, CCR4, CD16A, CD19, CD20, CD24, CD27, CD28, CD30, CD38, CD39, CD40, CD40L, CD47, CD52, CD70, CD80, CD86, CD123, CD133, CD137, CD137L, CD160, CD272, CEACAM5, CLEC9, CLEC91, CRTH2, CSF-1, CSF-2, CSF-3, CXCL1, CXCL2, CXCL4, CXCL12, CXCL13, CXCR3, cMet, CTLA4, DLL3, DLL4, DNGR-1, E-cadherin, EGFR, ENTPD1, EpCAM, FCER1, FCER1A, FCER2, FGFR, FLAP, FOLH1, Gi24, GITR, GITRL, GPR5, GP100, GPRC5D, HER2, HER3, ICOSL, ICOS, HHLA2, HMGB1, HVEM, IDO, IFNa, IgE, IGF1R, IL2Rbeta, IL1, ILIA, IL1B, IL1F10, IL2, IL4, IL4Ra, IL5, IL5R, IL6, IL7, IL7Ra, IL8, IL9, IL9R, IL10, rhILlO, IL12, IL13, IL13Ra1, IL13Ra2, IL15, IL17, IL17Rb, IL18, IL22, IL23, IL25, IL7, IL33, IL35, ITGB4, ITK, KIR, LAG3, LAMP1, leptin, LPFS2, MHC class II, MUC-1, MUC-16, NCR3LG1, NKG2D, NKp46, NTPDase-1, OX40, OX40L, PD-1, PD-L1, PD-L2, PROM1, S152, SIRPalpha, SISP1, SLC, SPG64, ST2, STEAP1, STEAP2, Syk kinase, STEAP1, TROP2, TACI, TDO, TGFBETA, T14, TIGIT, TIM3, TLR, TLR2, TLR4, TLR5, TLR9, TMEF1, TNFa, TNFRSF7, Tp55, TREM1, TSLP, TSLPR, TWEAK, VEGF, VISTA, Vstm3, or WUCAM; VH1 is a first immunoglobulin heavy chain variable region that specifically binds to A2AR, APRIL, ATPDase, BAFF, BAFFR, BCMA, BlyS, BTK, BTLA, B7DC, B7H1, B7H2, B7H3, B7H4, B7H5, B7H6, B7H7, B7RP1, B7-4, C3, C5, CCL2, CCL3, CCL4, CCL5, CCL7, CCL8, CCL11, CCL15, CCL17, CCL19, CCL20, CCL21, CCL25, CCR3, CCR4, CD16A, CD19, CD20, CD24, CD27, CD28, CD30, CD38, CD39, CD40, CD40L, CD47, CD52, CD70, CD80, CD86, CD123, CD133, CD137, CD137L, CD160, CD272, CEACAM5, CLEC9, CLEC91, CRTH2, CSF-1, CSF-2, CSF-3, CXCL1, CXCL2, CXCL4, CXCL12, CXCL13, CXCR3, cMet, CTLA4, DLL3, DLL4, DNGR-1, E-cadherin, EGFR, ENTPD1, EpCAM, FCER1, FCER1A, FCER2, FGFR, FLAP, FOLH1, Gi24, GITR, GITRL, GPR5, GP100, GPRC5D, HER2, HER3, ICOSL, ICOS, HHLA2, HMGB1, HVEM, IDO, IFNa, IgE, IGF1R, IL2Rbeta, IL1, ILIA, IL1B, IL1F10, IL2, IL4, IL4Ra, IL5, IL5R, IL6, IL7, IL7Ra, IL8, IL9, IL9R, IL10, rhILlO, IL12, IL13, IL13Ra1, IL13Ra2, IL15, IL17, IL17Rb, IL18, IL22, IL23, IL25, IL7, IL33, IL35, ITGB4, ITK, KIR, LAG3, LAMP1, leptin, LPFS2, MHC class II, MUC-1, MUC-16, NCR3LG1, NKG2D, NKp46, NTPDase-1, OX40, OX40L, PD-1, PD-L1, PD-L2, PROM1, S152, SIRPalpha, SISP1, SLC, SPG64, ST2, STEAP1, STEAP2, Syk kinase, STEAP1, TROP2, TACI, TDO, TGFBETA, T14, TIGIT, TIM3, TLR, TLR2, TLR4, TLR5, TLR9, TMEF1, TNFa, TNFRSF7, Tp55, TREM1, TSLP, TSLPR, TWEAK, VEGF, VISTA, Vstm3, or WUCAM; VH2 is a second immunoglobulin heavy chain variable region that specifically binds to A2AR, APRIL, ATPDase, BAFF, BAFFR, BCMA, BlyS, BTK, BTLA, B7DC, B7H1, B7H2, B7H3, B7H4, B7H5, B7H6, B7H7, B7RP1, B7-4, C3, C5, CCL2, CCL3, CCL4, CCL5, CCL7, CCL8, CCL11, CCL15, CCL17, CCL19, CCL20, CCL21, CCL25, CCR3, CCR4, CD16A, CD19, CD20, CD24, CD27, CD28, CD30, CD38, CD39, CD40, CD40L, CD47, CD52, CD70, CD80, CD86, CD123, CD133, CD137, CD137L, CD160, CD272, CEACAM5, CLEC9, CLEC91, CRTH2, CSF-1, CSF-2, CSF-3, CXCL1, CXCL2, CXCL4, CXCL12, CXCL13, CXCR3, cMet, CTLA4, DLL3, DLL4, DNGR-1, E-cadherin, EGFR, ENTPD1, EpCAM, FCER1, FCER1A, FCER2, FGFR, FLAP, FOLH1, Gi24, GITR, GITRL, GPR5, GP100, GPRC5D, HER2, HER3, ICOSL, ICOS, HHLA2, HMGB1, HVEM, IDO, IFNa, IgE, IGF1R, IL2Rbeta, IL1, ILIA, IL1B, IL1F10, IL2, IL4, IL4Ra, IL5, IL5R, IL6, IL7, IL7Ra, IL8, IL9, IL9R, IL10, rhILlO, IL12, IL13, IL13Ra1, IL13Ra2, IL15, IL17, IL17Rb, IL18, IL22, IL23, IL25, IL7, IL33, IL35, ITGB4, ITK, KIR, LAG3, LAMP1, leptin, LPFS2, MHC class II, MUC-1, MUC-16, NCR3LG1, NKG2D, NKp46, NTPDase-1, OX40, OX40L, PD-1, PD-L1, PD-L2, PROM1, S152, SIRPalpha, SISP1, SLC, SPG64, ST2, STEAP1, STEAP2, Syk kinase, STEAP1, TROP2, TACI, TDO, TGFBETA, T14, TIGIT, TIM3, TLR, TLR2, TLR4, TLR5, TLR9, TMEF1, TNFa, TNFRSF7, Tp55, TREM1, TSLP, TSLPR, TWEAK, VEGF, VISTA, Vstm3, or WUCAM; VH3 is a third immunoglobulin heavy chain variable region that specifically binds to CD3; VH4 is a fourth immunoglobulin heavy chain variable region that specifically binds to A2AR, APRIL, ATPDase, BAFF, BAFFR, BCMA, BlyS, BTK, BTLA, B7DC, B7H1, B7H2, B7H3, B7H4, B7H5, B7H6, B7H7, B7RP1, B7-4, C3, C5, CCL2, CCL3, CCL4, CCL5, CCL7, CCL8, CCL11, CCL15, CCL17, CCL19, CCL20, CCL21, CCL25, CCR3, CCR4, CD16A, CD19, CD20, CD24, CD27, CD28, CD30, CD38, CD39, CD40, CD40L, CD47, CD52, CD70, CD80, CD86, CD123, CD133, CD137, CD137L, CD160, CD272, CEACAM5, CLEC9, CLEC91, CRTH2, CSF-1, CSF-2, CSF-3, CXCL1, CXCL2, CXCL4, CXCL12, CXCL13, CXCR3, cMet, CTLA4, DLL3, DLL4, DNGR-1, E-cadherin, EGFR, ENTPD1, EpCAM, FCER1, FCER1A, FCER2, FGFR, FLAP, FOLH1, Gi24, GITR, GITRL, GPR5, GP100, GPRC5D, HER2, HER3, ICOSL, ICOS, HHLA2, HMGB1, HVEM, IDO, IFNa, IgE, IGF1R, IL2Rbeta, IL1, ILIA, IL1B, IL1F10, IL2, IL4, IL4Ra, IL5, IL5R, IL6, IL7, IL7Ra, IL8, IL9, IL9R, IL10, rhILlO, IL12, IL13, IL13Ra1, IL13Ra2, IL15, IL17, IL17Rb, IL18, IL22, IL23, IL25, IL7, IL33, IL35, ITGB4, ITK, KIR, LAG3, LAMP1, leptin, LPFS2, MHC class II, MUC-1, MUC-16, NCR3LG1, NKG2D, NKp46, NTPDase-1, OX40, OX40L, PD-1, PD-L1, PD-L2, PROM1, S152, SIRPalpha, SISP1, SLC, SPG64, ST2, STEAP1, STEAP2, Syk kinase, STEAP1, TROP2, TACI, TDO, TGFBETA, T14, TIGIT, TIM3, TLR, TLR2, TLR4, TLR5, TLR9, TMEF1, TNFa, TNFRSF7, Tp55, TREM1, TSLP, TSLPR, TWEAK, VEGF, VISTA, Vstm3, or WUCAM; Fe is a region comprising an immunoglobulin heavy chain constant region 2 (CH2), an immunoglobulin heavy chain constant region 3 (CH3), and optionally, an immunoglobulin hinge; and L1-L16 are amino acid linkers. In some aspects, the first polypeptide has a structure represented by VL1-L1-VL2-L2-VH2-L3-VH1-L4-Fc and the second polypeptide has a structure represented by VL3-L9-VL4-L10-VH4-L11-VH3-L12-Fc. In some aspects, the first polypeptide has a structure represented by VL1-L1-VL2-L2-VH2-L3-VH1-L4-Fc and the second polypeptide has a structure represented by VH3-L13-VH4-L14-VL4-L15-VL3-L16-Fc. In some aspects, the first polypeptide has a structure represented by VH1-L5-VH2-L6-VL2-L7-VL1-L8-Fc and the second polypeptide has a structure represented by VL3-L9-VL4-L10-VH4-L11-VH3-L12-Fc. In some aspects, the first polypeptide has a structure represented by VH1-L5-VH2-L6-VL2-L7-VL1-L8-Fc and the second polypeptide has a structure represented by VH3-L13-VH4-L14-VL4-L15-VL3-L16-Fc. In some aspects, VL1 and VH1 may specifically bind to A2AR, APRIL, ATPDase, BAFF, BAFFR, BCMA, BlyS, BTK, BTLA, B7DC, B7H1, B7H2, B7H3, B7H4, B7H5, B7H6, B7H7, B7RP1, B7-4, C3, C5, CCL2, CCL3, CCL4, CCL5, CCL7, CCL8, CCL11, CCL15, CCL17, CCL19, CCL20, CCL21, CCL25, CCR3, CCR4, CD16A, CD19, CD20, CD24, CD27, CD28, CD30, CD38, CD39, CD40, CD40L, CD47, CD52, CD70, CD80, CD86, CD123, CD133, CD137, CD137L, CD160, CD272, CEACAM5, CLEC9, CLEC91, CRTH2, CSF-1, CSF-2, CSF-3, CXCL1, CXCL2, CXCL4, CXCL12, CXCL13, CXCR3, cMet, CTLA4, DLL3, DLL4, DNGR-1, E-cadherin, EGFR, ENTPD1, EpCAM, FCER1, FCER1A, FCER2, FGFR, FLAP, FOLH1, Gi24, GITR, GITRL, GPR5, GP100, GPRC5D, HER2, HER3, ICOSL, ICOS, HHLA2, HMGB1, HVEM, IDO, IFNa, IgE, IGF1R, IL2Rbeta, IL1, ILIA, IL1B, IL1F10, IL2, IL4, IL4Ra, IL5, IL5R, IL6, IL7, IL7Ra, IL8, IL9, IL9R, IL10, rhILlO, IL12, IL13, IL13Ra1, IL13Ra2, IL15, IL17, IL17Rb, IL18, IL22, IL23, IL25, IL7, IL33, IL35, ITGB4, ITK, KIR, LAG3, LAMP1, leptin, LPFS2, MHC class II, MUC-1, MUC-16, NCR3LG1, NKG2D, NKp46, NTPDase-1, OX40, OX40L, PD-1, PD-L1, PD-L2, PROM1, S152, SIRPalpha, SISP1, SLC, SPG64, ST2, STEAP1, STEAP2, Syk kinase, STEAP1, TROP2, TACI, TDO, TGFBETA, T14, TIGIT, TIM3, TLR, TLR2, TLR4, TLR5, TLR9, TMEF1, TNFa, TNFRSF7, Tp55, TREM1, TSLP, TSLPR, TWEAK, VEGF, VISTA, Vstm3, or WUCAM. In some aspects, VL2 and VH2 may specifically bind to A2AR, APRIL, ATPDase, BAFF, BAFFR, BCMA, BlyS, BTK, BTLA, B7DC, B7H1, B7H2, B7H3, B7H4, B7H5, B7H6, B7H7, B7RP1, B7-4, C3, C5, CCL2, CCL3, CCL4, CCL5, CCL7, CCL8, CCL11, CCL15, CCL17, CCL19, CCL20, CCL21, CCL25, CCR3, CCR4, CD16A, CD19, CD20, CD24, CD27, CD28, CD30, CD38, CD39, CD40, CD40L, CD47, CD52, CD70, CD80, CD86, CD123, CD133, CD137, CD137L, CD160, CD272, CEACAM5, CLEC9, CLEC91, CRTH2, CSF-1, CSF-2, CSF-3, CXCL1, CXCL2, CXCL4, CXCL12, CXCL13, CXCR3, cMet, CTLA4, DLL3, DLL4, DNGR-1, E-cadherin, EGFR, ENTPD1, EpCAM, FCER1, FCER1A, FCER2, FGFR, FLAP, FOLH1, Gi24, GITR, GITRL, GPR5, GP100, GPRC5D, HER2, HER3, ICOSL, ICOS, HHLA2, HMGB1, HVEM, IDO, IFNa, IgE, IGF1R, IL2Rbeta, IL1, ILIA, IL1B, IL1F10, IL2, IL4, IL4Ra, IL5, IL5R, IL6, IL7, IL7Ra, IL8, IL9, IL9R, IL10, rhILlO, IL12, IL13, IL13Ra1, IL13Ra2, IL15, IL17, IL17Rb, IL18, IL22, IL23, IL25, IL7, IL33, IL35, ITGB4, ITK, KIR, LAG3, LAMP1, leptin, LPFS2, MHC class II, MUC-1, MUC-16, NCR3LG1, NKG2D, NKp46, NTPDase-1, OX40, OX40L, PD-1, PD-L1, PD-L2, PROM1, S152, SIRPalpha, SISP1, SLC, SPG64, ST2, STEAP1, STEAP2, Syk kinase, STEAP1, TROP2, TACI, TDO, TGFBETA, T14, TIGIT, TIM3, TLR, TLR2, TLR4, TLR5, TLR9, TMEF1, TNFa, TNFRSF7, Tp55, TREM1, TSLP, TSLPR, TWEAK, VEGF, VISTA, Vstm3, or WUCAM. In some aspects, VL4 and VH4 may specifically bind to A2AR, APRIL, ATPDase, BAFF, BAFFR, BCMA, BlyS, BTK, BTLA, B7DC, B7H1, B7H2, B7H3, B7H4, B7H5, B7H6, B7H7, B7RP1, B7-4, C3, C5, CCL2, CCL3, CCL4, CCL5, CCL7, CCL8, CCL11, CCL15, CCL17, CCL19, CCL20, CCL21, CCL25, CCR3, CCR4, CD16A, CD19, CD20, CD24, CD27, CD28, CD30, CD38, CD39, CD40, CD40L, CD47, CD52, CD70, CD80, CD86, CD123, CD133, CD137, CD137L, CD160, CD272, CEACAM5, CLEC9, CLEC91, CRTH2, CSF-1, CSF-2, CSF-3, CXCL1, CXCL2, CXCL4, CXCL12, CXCL13, CXCR3, cMet, CTLA4, DLL3, DLL4, DNGR-1, E-cadherin, EGFR, ENTPD1, EpCAM, FCER1, FCER1A, FCER2, FGFR, FLAP, FOLH1, Gi24, GITR, GITRL, GPR5, GP100, GPRC5D, HER2, HER3, ICOSL, ICOS, HHLA2, HMGB1, HVEM, IDO, IFNa, IgE, IGF1R, IL2Rbeta, IL1, ILIA, IL1B, IL1F10, IL2, IL4, IL4Ra, IL5, IL5R, IL6, IL7, IL7Ra, IL8, IL9, IL9R, IL10, rhILlO, IL12, IL13, IL13Ra1, IL13Ra2, IL15, IL17, IL17Rb, IL18, IL22, IL23, IL25, IL7, IL33, IL35, ITGB4, ITK, KIR, LAG3, LAMP1, leptin, LPFS2, MHC class II, MUC-1, MUC-16, NCR3LG1, NKG2D, NKp46, NTPDase-1, OX40, OX40L, PD-1, PD-L1, PD-L2, PROM1, S152, SIRPalpha, SISP1, SLC, SPG64, ST2, STEAP1, STEAP2, Syk kinase, STEAP1, TROP2, TACI, TDO, TGFBETA, T14, TIGIT, TIM3, TLR, TLR2, TLR4, TLR5, TLR9, TMEF1, TNFa, TNFRSF7, Tp55, TREM1, TSLP, TSLPR, TWEAK, VEGF, VISTA, Vstm3, or WUCAM. For example, the antigen binding polypeptide complex may specifically bind CD28, CD3, cMet, and Trop2. For example, the antigen binding polypeptide complex may specifically bind CD28, CD3, CD19, and CD20. For example, VL1 and VH1 may specifically bind to CD28. For example, VL2 and VH2 may specifically bind to CD28. For example, VL4 and VH4 may specifically bind to CD28. For example, VL1 and VH1 may specifically bind to cMet. For example, VL2 and VH2 may specifically bind to cMet. For example, VL4 and VH4 may specifically bind to cMet. For example, VL1 and VH1 may specifically bind to Trop2. For example, VL2 and VH2 may specifically bind to Trop2. For example, VL4 and VH4 may specifically bind to Trop2. For example, VL1 and VH1 may specifically bind to CD19. For example, VL2 and VH2 may specifically bind to CD19. For example, VL4 and VH4 may specifically bind to CD19. For example, VL1 and VH1 may specifically bind to CD20. For example, VL2 and VH2 may specifically bind to CD20. For example, VL4 and VH4 may specifically bind to CD20.

In some aspects, the invention is directed to an antigen binding polypeptide complex comprising a first polypeptide and a second polypeptide; wherein the first polypeptide has a structure represented by VL1-L1-VL2-L2-VH2-L3-VH1-L4-Fc; or VH1-L5-VH2-L6-VL2-L7-VL1-L8-Fc; wherein the second polypeptide has a structure represented by VL3-L9-VL4-L10-VH4-L11-VH3-L12-Fc; or VH3-L13-VH4-L14-VL4-L15-VL3-L16-Fc; wherein VL1 is a first immunoglobulin light chain variable region that specifically binds to CD28; VL2 is a second immunoglobulin light chain variable region that specifically binds to A2AR, APRIL, ATPDase, BAFF, BAFFR, BCMA, BlyS, BTK, BTLA, B7DC, B7H1, B7H2, B7H3, B7H4, B7H5, B7H6, B7H7, B7RP1, B7-4, C3, C5, CCL2, CCL3, CCL4, CCL5, CCL7, CCL8, CCL11, CCL15, CCL17, CCL19, CCL20, CCL21, CCL25, CCR3, CCR4, CD16A, CD19, CD20, CD24, CD27, CD28, CD30, CD38, CD39, CD40, CD40L, CD47, CD52, CD70, CD80, CD86, CD123, CD133, CD137, CD137L, CD160, CD272, CEACAM5, CLEC9, CLEC91, CRTH2, CSF-1, CSF-2, CSF-3, CXCL1, CXCL2, CXCL4, CXCL12, CXCL13, CXCR3, cMet, CTLA4, DLL3, DLL4, DNGR-1, E-cadherin, EGFR, ENTPD1, EpCAM, FCER1, FCER1A, FCER2, FGFR, FLAP, FOLH1, Gi24, GITR, GITRL, GPR5, GP100, GPRC5D, HER2, HER3, ICOSL, ICOS, HHLA2, HMGB1, HVEM, IDO, IFNa, IgE, IGF1R, IL2Rbeta, IL1, ILIA, IL1B, IL1F10, IL2, IL4, IL4Ra, IL5, IL5R, IL6, IL7, IL7Ra, IL8, IL9, IL9R, IL10, rhILlO, IL12, IL13, IL13Ra1, IL13Ra2, IL15, IL17, IL17Rb, IL18, IL22, IL23, IL25, IL7, IL33, IL35, ITGB4, ITK, KIR, LAG3, LAMP1, leptin, LPFS2, MHC class II, MUC-1, MUC-16, NCR3LG1, NKG2D, NKp46, NTPDase-1, OX40, OX40L, PD-1, PD-L1, PD-L2, PROM1, S152, SIRPalpha, SISP1, SLC, SPG64, ST2, STEAP1, STEAP2, Syk kinase, STEAP1, TROP2, TACI, TDO, TGFBETA, T14, TIGIT, TIM3, TLR, TLR2, TLR4, TLR5, TLR9, TMEF1, TNFa, TNFRSF7, Tp55, TREM1, TSLP, TSLPR, TWEAK, VEGF, VISTA, Vstm3, or WUCAM; VL3 is a third immunoglobulin light chain variable region that specifically binds to CD3; VL4 is a fourth immunoglobulin light chain variable region that specifically binds to A2AR, APRIL, ATPDase, BAFF, BAFFR, BCMA, BlyS, BTK, BTLA, B7DC, B7H1, B7H2, B7H3, B7H4, B7H5, B7H6, B7H7, B7RP1, B7-4, C3, C5, CCL2, CCL3, CCL4, CCL5, CCL7, CCL8, CCL11, CCL15, CCL17, CCL19, CCL20, CCL21, CCL25, CCR3, CCR4, CD16A, CD19, CD20, CD24, CD27, CD28, CD30, CD38, CD39, CD40, CD40L, CD47, CD52, CD70, CD80, CD86, CD123, CD133, CD137, CD137L, CD160, CD272, CEACAM5, CLEC9, CLEC91, CRTH2, CSF-1, CSF-2, CSF-3, CXCL1, CXCL2, CXCL4, CXCL12, CXCL13, CXCR3, cMet, CTLA4, DLL3, DLL4, DNGR-1, E-cadherin, EGFR, ENTPD1, EpCAM, FCER1, FCER1A, FCER2, FGFR, FLAP, FOLH1, Gi24, GITR, GITRL, GPR5, GP100, GPRC5D, HER2, HER3, ICOSL, ICOS, HHLA2, HMGB1, HVEM, IDO, IFNa, IgE, IGF1R, IL2Rbeta, IL1, ILIA, IL1B, IL1F10, IL2, IL4, IL4Ra, IL5, IL5R, IL6, IL7, IL7Ra, IL8, IL9, IL9R, IL10, rhILlO, IL12, IL13, IL13Ra1, IL13Ra2, IL15, IL17, IL17Rb, IL18, IL22, IL23, IL25, IL7, IL33, IL35, ITGB4, ITK, KIR, LAG3, LAMP1, leptin, LPFS2, MHC class II, MUC-1, MUC-16, NCR3LG1, NKG2D, NKp46, NTPDase-1, OX40, OX40L, PD-1, PD-L1, PD-L2, PROM1, 5152, SIRPalpha, SISP1, SLC, SPG64, ST2, STEAP1, STEAP2, Syk kinase, STEAP1, TROP2, TACI, TDO, TGFBETA, T14, TIGIT, TIM3, TLR, TLR2, TLR4, TLR5, TLR9, TMEF1, TNFa, TNFRSF7, Tp55, TREM1, TSLP, TSLPR, TWEAK, VEGF, VISTA, Vstm3, or WUCAM; VH1 is a first immunoglobulin heavy chain variable region that specifically binds to CD28; VH2 is a second immunoglobulin heavy chain variable region that specifically binds to A2AR, APRIL, ATPDase, BAFF, BAFFR, BCMA, BlyS, BTK, BTLA, B7DC, B7H1, B7H2, B7H3, B7H4, B7H5, B7H6, B7H7, B7RP1, B7-4, C3, C5, CCL2, CCL3, CCL4, CCL5, CCL7, CCL8, CCL11, CCL15, CCL17, CCL19, CCL20, CCL21, CCL25, CCR3, CCR4, CD16A, CD19, CD20, CD24, CD27, CD28, CD30, CD38, CD39, CD40, CD40L, CD47, CD52, CD70, CD80, CD86, CD123, CD133, CD137, CD137L, CD160, CD272, CEACAM5, CLEC9, CLEC91, CRTH2, CSF-1, CSF-2, CSF-3, CXCL1, CXCL2, CXCL4, CXCL12, CXCL13, CXCR3, cMet, CTLA4, DLL3, DLL4, DNGR-1, E-cadherin, EGFR, ENTPD1, EpCAM, FCER1, FCER1A, FCER2, FGFR, FLAP, FOLH1, Gi24, GITR, GITRL, GPR5, GP100, GPRC5D, HER2, HER3, ICOSL, ICOS, HHLA2, HMGB1, HVEM, IDO, IFNa, IgE, IGF1R, IL2Rbeta, IL1, ILIA, IL1B, IL1F10, IL2, IL4, IL4Ra, IL5, IL5R, IL6, IL7, IL7Ra, IL8, IL9, IL9R, IL10, rhILlO, IL12, IL13, IL13Ra1, IL13Ra2, IL15, IL17, IL17Rb, IL18, IL22, IL23, IL25, IL7, IL33, IL35, ITGB4, ITK, KIR, LAG3, LAMP1, leptin, LPFS2, MHC class II, MUC-1, MUC-16, NCR3LG1, NKG2D, NKp46, NTPDase-1, OX40, OX40L, PD-1, PD-L1, PD-L2, PROM1, S152, SIRPalpha, SISP1, SLC, SPG64, ST2, STEAP1, STEAP2, Syk kinase, STEAP1, TROP2, TACI, TDO, TGFBETA, T14, TIGIT, TIM3, TLR, TLR2, TLR4, TLR5, TLR9, TMEF1, TNFa, TNFRSF7, Tp55, TREM1, TSLP, TSLPR, TWEAK, VEGF, VISTA, Vstm3, or WUCAM; VH3 is a third immunoglobulin heavy chain variable region that specifically binds to CD3; VH4 is a fourth immunoglobulin heavy chain variable region that specifically binds to A2AR, APRIL, ATPDase, BAFF, BAFFR, BCMA, BlyS, BTK, BTLA, B7DC, B7H1, B7H2, B7H3, B7H4, B7H5, B7H6, B7H7, B7RP1, B7-4, C3, C5, CCL2, CCL3, CCL4, CCL5, CCL7, CCL8, CCL11, CCL15, CCL17, CCL19, CCL20, CCL21, CCL25, CCR3, CCR4, CD16A, CD19, CD20, CD24, CD27, CD28, CD30, CD38, CD39, CD40, CD40L, CD47, CD52, CD70, CD80, CD86, CD123, CD133, CD137, CD137L, CD160, CD272, CEACAM5, CLEC9, CLEC91, CRTH2, CSF-1, CSF-2, CSF-3, CXCL1, CXCL2, CXCL4, CXCL12, CXCL13, CXCR3, cMet, CTLA4, DLL3, DLL4, DNGR-1, E-cadherin, EGFR, ENTPD1, EpCAM, FCER1, FCER1A, FCER2, FGFR, FLAP, FOLH1, Gi24, GITR, GITRL, GPR5, GP100, GPRC5D, HER2, HER3, ICOSL, ICOS, HHLA2, HMGB1, HVEM, IDO, IFNa, IgE, IGF1R, IL2Rbeta, IL1, ILIA, IL1B, IL1F10, IL2, IL4, IL4Ra, IL5, IL5R, IL6, IL7, IL7Ra, IL8, IL9, IL9R, IL10, rhILlO, IL12, IL13, IL13Ra1, IL13Ra2, IL15, IL17, IL17Rb, IL18, IL22, IL23, IL25, IL7, IL33, IL35, ITGB4, ITK, KIR, LAG3, LAMP1, leptin, LPFS2, MHC class II, MUC-1, MUC-16, NCR3LG1, NKG2D, NKp46, NTPDase-1, OX40, OX40L, PD-1, PD-L1, PD-L2, PROM1, S152, SIRPalpha, SISP1, SLC, SPG64, ST2, STEAP1, STEAP2, Syk kinase, STEAP1, TROP2, TACI, TDO, TGFBETA, T14, TIGIT, TIM3, TLR, TLR2, TLR4, TLR5, TLR9, TMEF1, TNFa, TNFRSF7, Tp55, TREM1, TSLP, TSLPR, TWEAK, VEGF, VISTA, Vstm3, or WUCAM; Fc is a region comprising an immunoglobulin heavy chain constant region 2 (CH2), an immunoglobulin heavy chain constant region 3 (CH3), and optionally, an immunoglobulin hinge; and L1-L16 are amino acid linkers. In some aspects, the first polypeptide has a structure represented by VL1-L1-VL2-L2-VH2-L3-VH1-L4-Fc and the second polypeptide has a structure represented by VL3-L9-VL4-L10-VH4-L11-VH3-L12-Fc. In some aspects, the first polypeptide has a structure represented by VL1-L1-VL2-L2-VH2-L3-VH1-L4-Fc and the second polypeptide has a structure represented by VH3-L13-VH4-L14-VL4-L15-VL3-L16-Fc. In some aspects, the first polypeptide has a structure represented by VH1-L5-VH2-L6-VL2-L7-VL1-L8-Fc and the second polypeptide has a structure represented by VL3-L9-VL4-L10-VH4-L11-VH3-L12-Fc. In some aspects, the first polypeptide has a structure represented by VH1-L5-VH2-L6-VL2-L7-VL1-L8-Fc and the second polypeptide has a structure represented by VH3-L13-VH4-L14-VL4-L15-VL3-L16-Fc. In some aspects, VL2 and VH2 may specifically bind to A2AR, APRIL, ATPDase, BAFF, BAFFR, BCMA, BlyS, BTK, BTLA, B7DC, B7H1, B7H2, B7H3, B7H4, B7H5, B7H6, B7H7, B7RP1, B7-4, C3, C5, CCL2, CCL3, CCL4, CCL5, CCL7, CCL8, CCL11, CCL15, CCL17, CCL19, CCL20, CCL21, CCL25, CCR3, CCR4, CD16A, CD19, CD20, CD24, CD27, CD28, CD30, CD38, CD39, CD40, CD40L, CD47, CD52, CD70, CD80, CD86, CD123, CD133, CD137, CD137L, CD160, CD272, CEACAM5, CLEC9, CLEC91, CRTH2, CSF-1, CSF-2, CSF-3, CXCL1, CXCL2, CXCL4, CXCL12, CXCL13, CXCR3, cMet, CTLA4, DLL3, DLL4, DNGR-1, E-cadherin, EGFR, ENTPD1, EpCAM, FCER1, FCER1A, FCER2, FGFR, FLAP, FOLH1, Gi24, GITR, GITRL, GPR5, GP100, GPRC5D, HER2, HER3, ICOSL, ICOS, HHLA2, HMGB1, HVEM, IDO, IFNa, IgE, IGF1R, IL2Rbeta, IL1, ILIA, IL1B, IL1F10, IL2, IL4, IL4Ra, IL5, IL5R, IL6, IL7, IL7Ra, IL8, IL9, IL9R, IL10, rhILlO, IL12, IL13, IL13Ra1, IL13Ra2, IL15, IL17, IL17Rb, IL18, IL22, IL23, IL25, IL7, IL33, IL35, ITGB4, ITK, KIR, LAG3, LAMP1, leptin, LPFS2, MHC class II, MUC-1, MUC-16, NCR3LG1, NKG2D, NKp46, NTPDase-1, OX40, OX40L, PD-1, PD-L1, PD-L2, PROM1, S152, SIRPalpha, SISP1, SLC, SPG64, ST2, STEAP1, STEAP2, Syk kinase, STEAP1, TROP2, TACI, TDO, TGFBETA, T14, TIGIT, TIM3, TLR, TLR2, TLR4, TLR5, TLR9, TMEF1, TNFa, TNFRSF7, Tp55, TREM1, TSLP, TSLPR, TWEAK, VEGF, VISTA, Vstm3, or WUCAM. In some aspects, VL4 and VH4 may specifically bind to A2AR, APRIL, ATPDase, BAFF, BAFFR, BCMA, BlyS, BTK, BTLA, B7DC, B7H1, B7H2, B7H3, B7H4, B7H5, B7H6, B7H7, B7RP1, B7-4, C3, C5, CCL2, CCL3, CCL4, CCL5, CCL7, CCL8, CCL11, CCL15, CCL17, CCL19, CCL20, CCL21, CCL25, CCR3, CCR4, CD16A, CD19, CD20, CD24, CD27, CD28, CD30, CD38, CD39, CD40, CD40L, CD47, CD52, CD70, CD80, CD86, CD123, CD133, CD137, CD137L, CD160, CD272, CEACAM5, CLEC9, CLEC91, CRTH2, CSF-1, CSF-2, CSF-3, CXCL1, CXCL2, CXCL4, CXCL12, CXCL13, CXCR3, cMet, CTLA4, DLL3, DLL4, DNGR-1, E-cadherin, EGFR, ENTPD1, EpCAM, FCER1, FCER1A, FCER2, FGFR, FLAP, FOLH1, Gi24, GITR, GITRL, GPR5, GP100, GPRC5D, HER2, HER3, ICOSL, ICOS, HHLA2, HMGB1, HVEM, IDO, IFNa, IgE, IGF1R, IL2Rbeta, IL1, ILIA, IL1B, IL1F10, IL2, IL4, IL4Ra, IL5, IL5R, IL6, IL7, IL7Ra, IL8, IL9, IL9R, IL10, rhILlO, IL12, IL13, IL13Ra1, IL13Ra2, IL15, IL17, IL17Rb, IL18, IL22, IL23, IL25, IL7, IL33, IL35, ITGB4, ITK, KIR, LAG3, LAMP1, leptin, LPFS2, MHC class II, MUC-1, MUC-16, NCR3LG1, NKG2D, NKp46, NTPDase-1, OX40, OX40L, PD-1, PD-L1, PD-L2, PROM1, S152, SIRPalpha, SISP1, SLC, SPG64, ST2, STEAP1, STEAP2, Syk kinase, STEAP1, TROP2, TACI, TDO, TGFBETA, T14, TIGIT, TIM3, TLR, TLR2, TLR4, TLR5, TLR9, TMEF1, TNFa, TNFRSF7, Tp55, TREM1, TSLP, TSLPR, TWEAK, VEGF, VISTA, Vstm3, or WUCAM. For example, the antigen binding polypeptide complex may specifically bind CD28, CD3, cMet, and Trop2. For example, the antigen binding polypeptide complex may specifically bind CD28, CD3, CD19, and CD20. For example, VL2 and VH2 may specifically bind to cMet. For example, VL4 and VH4 may specifically bind to cMet. For example, VL2 and VH2 may specifically bind to Trop2. For example, VL4 and VH4 may specifically bind to Trop2. For example, VL2 and VH2 may specifically bind to CD19. For example, VL4 and VH4 may specifically bind to CD19. For example, VL2 and VH2 may specifically bind to CD20. For example, VL4 and VH4 may specifically bind to CD20.

In some aspects, the invention is directed to an antigen binding polypeptide complex comprising a first polypeptide and a second polypeptide; wherein the first polypeptide has a structure represented by VL1-L1-VL2-L2-VH2-L3-VH1-L4-Fc; or VH1-L5-VH2-L6-VL2-L7-VL1-L8-Fc; wherein the second polypeptide has a structure represented by VL3-L9-VL4-L10-VH4-L11-VH3-L12-Fc; or VH3-L13-VH4-L14-VL4-L15-VL3-L16-Fc; wherein VL1 is a first immunoglobulin light chain variable region that specifically binds to A2AR, APRIL, ATPDase, BAFF, BAFFR, BCMA, BlyS, BTK, BTLA, B7DC, B7H1, B7H2, B7H3, B7H4, B7H5, B7H6, B7H7, B7RP1, B7-4, C3, C5, CCL2, CCL3, CCL4, CCL5, CCL7, CCL8, CCL11, CCL15, CCL17, CCL19, CCL20, CCL21, CCL25, CCR3, CCR4, CD16A, CD19, CD20, CD24, CD27, CD28, CD30, CD38, CD39, CD40, CD40L, CD47, CD52, CD70, CD80, CD86, CD123, CD133, CD137, CD137L, CD160, CD272, CEACAM5, CLEC9, CLEC91, CRTH2, CSF-1, CSF-2, CSF-3, CXCL1, CXCL2, CXCL4, CXCL12, CXCL13, CXCR3, cMet, CTLA4, DLL3, DLL4, DNGR-1, E-cadherin, EGFR, ENTPD1, EpCAM, FCER1, FCER1A, FCER2, FGFR, FLAP, FOLH1, Gi24, GITR, GITRL, GPR5, GP100, GPRC5D, HER2, HER3, ICOSL, ICOS, HHLA2, HMGB1, HVEM, IDO, IFNa, IgE, IGF1R, IL2Rbeta, IL1, ILIA, IL1B, IL1F10, IL2, IL4, IL4Ra, IL5, IL5R, IL6, IL7, IL7Ra, IL8, IL9, IL9R, IL10, rhILlO, IL12, IL13, IL13Ra1, IL13Ra2, IL15, IL17, IL17Rb, IL18, IL22, IL23, IL25, IL7, IL33, IL35, ITGB4, ITK, KIR, LAG3, LAMP1, leptin, LPFS2, MHC class II, MUC-1, MUC-16, NCR3LG1, NKG2D, NKp46, NTPDase-1, OX40, OX40L, PD-1, PD-L1, PD-L2, PROM1, S152, SIRPalpha, SISP1, SLC, SPG64, ST2, STEAP1, STEAP2, Syk kinase, STEAP1, TROP2, TACI, TDO, TGFBETA, T14, TIGIT, TIM3, TLR, TLR2, TLR4, TLR5, TLR9, TMEF1, TNFa, TNFRSF7, Tp55, TREM1, TSLP, TSLPR, TWEAK, VEGF, VISTA, Vstm3, or WUCAM; VL2 is a second immunoglobulin light chain variable region that specifically binds to CD28; VL3 is a third immunoglobulin light chain variable region that specifically binds to CD3; VL4 is a fourth immunoglobulin light chain variable region that specifically binds to A2AR, APRIL, ATPDase, BAFF, BAFFR, BCMA, BlyS, BTK, BTLA, B7DC, B7H1, B7H2, B7H3, B7H4, B7H5, B7H6, B7H7, B7RP1, B7-4, C3, C5, CCL2, CCL3, CCL4, CCL5, CCL7, CCL8, CCL11, CCL15, CCL17, CCL19, CCL20, CCL21, CCL25, CCR3, CCR4, CD16A, CD19, CD20, CD24, CD27, CD28, CD30, CD38, CD39, CD40, CD40L, CD47, CD52, CD70, CD80, CD86, CD123, CD133, CD137, CD137L, CD160, CD272, CEACAM5, CLEC9, CLEC91, CRTH2, CSF-1, CSF-2, CSF-3, CXCL1, CXCL2, CXCL4, CXCL12, CXCL13, CXCR3, cMet, CTLA4, DLL3, DLL4, DNGR-1, E-cadherin, EGFR, ENTPD1, EpCAM, FCER1, FCER1A, FCER2, FGFR, FLAP, FOLH1, Gi24, GITR, GITRL, GPR5, GP100, GPRC5D, HER2, HER3, ICOSL, ICOS, HHLA2, HMGB1, HVEM, IDO, IFNa, IgE, IGF1R, IL2Rbeta, IL1, ILIA, IL1B, IL1F10, IL2, IL4, IL4Ra, IL5, IL5R, IL6, IL7, IL7Ra, IL8, IL9, IL9R, IL10, rhILlO, IL12, IL13, IL13Ra1, IL13Ra2, IL15, IL17, IL17Rb, IL18, IL22, IL23, IL25, IL7, IL33, IL35, ITGB4, ITK, KIR, LAG3, LAMP1, leptin, LPFS2, MHC class II, MUC-1, MUC-16, NCR3LG1, NKG2D, NKp46, NTPDase-1, OX40, OX40L, PD-1, PD-L1, PD-L2, PROM1, S152, SIRPalpha, SISP1, SLC, SPG64, ST2, STEAP1, STEAP2, Syk kinase, STEAP1, TROP2, TACI, TDO, TGFBETA, T14, TIGIT, TIM3, TLR, TLR2, TLR4, TLR5, TLR9, TMEF1, TNFa, TNFRSF7, Tp55, TREM1, TSLP, TSLPR, TWEAK, VEGF, VISTA, Vstm3, or WUCAM; VH1 is a first immunoglobulin heavy chain variable region that specifically binds to A2AR, APRIL, ATPDase, BAFF, BAFFR, BCMA, BlyS, BTK, BTLA, B7DC, B7H1, B7H2, B7H3, B7H4, B7H5, B7H6, B7H7, B7RP1, B7-4, C3, C5, CCL2, CCL3, CCL4, CCL5, CCL7, CCL8, CCL11, CCL15, CCL17, CCL19, CCL20, CCL21, CCL25, CCR3, CCR4, CD16A, CD19, CD20, CD24, CD27, CD28, CD30, CD38, CD39, CD40, CD40L, CD47, CD52, CD70, CD80, CD86, CD123, CD133, CD137, CD137L, CD160, CD272, CEACAM5, CLEC9, CLEC91, CRTH2, CSF-1, CSF-2, CSF-3, CXCL1, CXCL2, CXCL4, CXCL12, CXCL13, CXCR3, cMet, CTLA4, DLL3, DLL4, DNGR-1, E-cadherin, EGFR, ENTPD1, EpCAM, FCER1, FCER1A, FCER2, FGFR, FLAP, FOLH1, Gi24, GITR, GITRL, GPR5, GP100, GPRC5D, HER2, HER3, ICOSL, ICOS, HHLA2, HMGB1, HVEM, IDO, IFNa, IgE, IGF1R, IL2Rbeta, IL1, ILIA, IL1B, IL1F10, IL2, IL4, IL4Ra, IL5, IL5R, IL6, IL7, IL7Ra, IL8, IL9, IL9R, IL10, rhILlO, IL12, IL13, IL13Ra1, IL13Ra2, IL15, IL17, IL17Rb, IL18, IL22, IL23, IL25, IL7, IL33, IL35, ITGB4, ITK, KIR, LAG3, LAMP1, leptin, LPFS2, MHC class II, MUC-1, MUC-16, NCR3LG1, NKG2D, NKp46, NTPDase-1, OX40, OX40L, PD-1, PD-L1, PD-L2, PROM1, S152, SIRPalpha, SISP1, SLC, SPG64, ST2, STEAP1, STEAP2, Syk kinase, STEAP1, TROP2, TACI, TDO, TGFBETA, T14, TIGIT, TIM3, TLR, TLR2, TLR4, TLR5, TLR9, TMEF1, TNFa, TNFRSF7, Tp55, TREM1, TSLP, TSLPR, TWEAK, VEGF, VISTA, Vstm3, or WUCAM; VH2 is a second immunoglobulin heavy chain variable region that specifically binds to CD28; VH3 is a third immunoglobulin heavy chain variable region that specifically binds to CD3; VH4 is a fourth immunoglobulin heavy chain variable region that specifically binds to A2AR, APRIL, ATPDase, BAFF, BAFFR, BCMA, BlyS, BTK, BTLA, B7DC, B7H1, B7H2, B7H3, B7H4, B7H5, B7H6, B7H7, B7RP1, B7-4, C3, C5, CCL2, CCL3, CCL4, CCL5, CCL7, CCL8, CCL11, CCL15, CCL17, CCL19, CCL20, CCL21, CCL25, CCR3, CCR4, CD16A, CD19, CD20, CD24, CD27, CD28, CD30, CD38, CD39, CD40, CD40L, CD47, CD52, CD70, CD80, CD86, CD123, CD133, CD137, CD137L, CD160, CD272, CEACAM5, CLEC9, CLEC91, CRTH2, CSF-1, CSF-2, CSF-3, CXCL1, CXCL2, CXCL4, CXCL12, CXCL13, CXCR3, cMet, CTLA4, DLL3, DLL4, DNGR-1, E-cadherin, EGFR, ENTPD1, EpCAM, FCER1, FCER1A, FCER2, FGFR, FLAP, FOLH1, Gi24, GITR, GITRL, GPR5, GP100, GPRC5D, HER2, HER3, ICOSL, ICOS, HHLA2, HMGB1, HVEM, IDO, IFNa, IgE, IGF1R, IL2Rbeta, IL1, ILIA, IL1B, IL1F10, IL2, IL4, IL4Ra, IL5, IL5R, IL6, IL7, IL7Ra, IL8, IL9, IL9R, IL10, rhILlO, IL12, IL13, IL13Ra1, IL13Ra2, IL15, IL17, IL17Rb, IL18, IL22, IL23, IL25, IL7, IL33, IL35, ITGB4, ITK, KIR, LAG3, LAMP1, leptin, LPFS2, MHC class II, MUC-1, MUC-16, NCR3LG1, NKG2D, NKp46, NTPDase-1, OX40, OX40L, PD-1, PD-L1, PD-L2, PROM1, S152, SIRPalpha, SISP1, SLC, SPG64, ST2, STEAP1, STEAP2, Syk kinase, STEAP1, TROP2, TACI, TDO, TGFBETA, T14, TIGIT, TIM3, TLR, TLR2, TLR4, TLR5, TLR9, TMEF1, TNFa, TNFRSF7, Tp55, TREM1, TSLP, TSLPR, TWEAK, VEGF, VISTA, Vstm3, or WUCAM; Fc is a region comprising an immunoglobulin heavy chain constant region 2 (CH2), an immunoglobulin heavy chain constant region 3 (CH3), and optionally, an immunoglobulin hinge; and L1-L16 are amino acid linkers. In some aspects, the first polypeptide has a structure represented by VL1-L1-VL2-L2-VH2-L3-VH1-L4-Fc and the second polypeptide has a structure represented by VL3-L9-VL4-L10-VH4-L11-VH3-L12-Fc. In some aspects, the first polypeptide has a structure represented by VL1-L1-VL2-L2-VH2-L3-VH1-L4-Fc and the second polypeptide has a structure represented by VH3-L13-VH4-L14-VL4-L15-VL3-L16-Fc. In some aspects, the first polypeptide has a structure represented by VH1-L5-VH2-L6-VL2-L7-VL1-L8-Fc and the second polypeptide has a structure represented by VL3-L9-VL4-L10-VH4-L11-VH3-L12-Fc. In some aspects, the first polypeptide has a structure represented by VH1-L5-VH2-L6-VL2-L7-VL1-L8-Fc and the second polypeptide has a structure represented by VH3-L13-VH4-L14-VL4-L15-VL3-L16-Fc. In some aspects, VL1 and VH1 may specifically bind to A2AR, APRIL, ATPDase, BAFF, BAFFR, BCMA, BlyS, BTK, BTLA, B7DC, B7H1, B7H2, B7H3, B7H4, B7H5, B7H6, B7H7, B7RP1, B7-4, C3, C5, CCL2, CCL3, CCL4, CCL5, CCL7, CCL8, CCL11, CCL15, CCL17, CCL19, CCL20, CCL21, CCL25, CCR3, CCR4, CD16A, CD19, CD20, CD24, CD27, CD28, CD30, CD38, CD39, CD40, CD40L, CD47, CD52, CD70, CD80, CD86, CD123, CD133, CD137, CD137L, CD160, CD272, CEACAM5, CLEC9, CLEC91, CRTH2, CSF-1, CSF-2, CSF-3, CXCL1, CXCL2, CXCL4, CXCL12, CXCL13, CXCR3, cMet, CTLA4, DLL3, DLL4, DNGR-1, E-cadherin, EGFR, ENTPD1, EpCAM, FCER1, FCER1A, FCER2, FGFR, FLAP, FOLH1, Gi24, GITR, GITRL, GPR5, GP100, GPRC5D, HER2, HER3, ICOSL, ICOS, HHLA2, HMGB1, HVEM, IDO, IFNa, IgE, IGF1R, IL2Rbeta, IL1, ILIA, IL1B, IL1F10, IL2, IL4, IL4Ra, IL5, IL5R, IL6, IL7, IL7Ra, IL8, IL9, IL9R, IL10, rhILlO, IL12, IL13, IL13Ra1, IL13Ra2, IL15, IL17, IL17Rb, IL18, IL22, IL23, IL25, IL7, IL33, IL35, ITGB4, ITK, KIR, LAG3, LAMP1, leptin, LPFS2, MHC class II, MUC-1, MUC-16, NCR3LG1, NKG2D, NKp46, NTPDase-1, OX40, OX40L, PD-1, PD-L1, PD-L2, PROM1, S152, SIRPalpha, SISP1, SLC, SPG64, ST2, STEAP1, STEAP2, Syk kinase, STEAP1, TROP2, TACI, TDO, TGFBETA, T14, TIGIT, TIM3, TLR, TLR2, TLR4, TLR5, TLR9, TMEF1, TNFa, TNFRSF7, Tp55, TREM1, TSLP, TSLPR, TWEAK, VEGF, VISTA, Vstm3, or WUCAM. In some aspects, VL4 and VH4 may specifically bind to A2AR, APRIL, ATPDase, BAFF, BAFFR, BCMA, BlyS, BTK, BTLA, B7DC, B7H1, B7H2, B7H3, B7H4, B7H5, B7H6, B7H7, B7RP1, B7-4, C3, C5, CCL2, CCL3, CCL4, CCL5, CCL7, CCL8, CCL11, CCL15, CCL17, CCL19, CCL20, CCL21, CCL25, CCR3, CCR4, CD16A, CD19, CD20, CD24, CD27, CD28, CD30, CD38, CD39, CD40, CD40L, CD47, CD52, CD70, CD80, CD86, CD123, CD133, CD137, CD137L, CD160, CD272, CEACAM5, CLEC9, CLEC91, CRTH2, CSF-1, CSF-2, CSF-3, CXCL1, CXCL2, CXCL4, CXCL12, CXCL13, CXCR3, cMet, CTLA4, DLL3, DLL4, DNGR-1, E-cadherin, EGFR, ENTPD1, EpCAM, FCER1, FCER1A, FCER2, FGFR, FLAP, FOLH1, Gi24, GITR, GITRL, GPR5, GP100, GPRC5D, HER2, HER3, ICOSL, ICOS, HHLA2, HMGB1, HVEM, IDO, IFNa, IgE, IGF1R, IL2Rbeta, IL1, ILIA, IL1B, IL1F10, IL2, IL4, IL4Ra, IL5, IL5R, IL6, IL7, IL7Ra, IL8, IL9, IL9R, IL10, rhILlO, IL12, IL13, IL13Ra1, IL13Ra2, IL15, IL17, IL17Rb, IL18, IL22, IL23, IL25, IL7, IL33, IL35, ITGB4, ITK, KIR, LAG3, LAMP1, leptin, LPFS2, MHC class II, MUC-1, MUC-16, NCR3LG1, NKG2D, NKp46, NTPDase-1, OX40, OX40L, PD-1, PD-L1, PD-L2, PROM1, S152, SIRPalpha, SISP1, SLC, SPG64, ST2, STEAP1, STEAP2, Syk kinase, STEAP1, TROP2, TACI, TDO, TGFBETA, T14, TIGIT, TIM3, TLR, TLR2, TLR4, TLR5, TLR9, TMEF1, TNFa, TNFRSF7, Tp55, TREM1, TSLP, TSLPR, TWEAK, VEGF, VISTA, Vstm3, or WUCAM. For example, the antigen binding polypeptide complex may specifically bind CD28, CD3, cMet, and Trop2. For example, the antigen binding polypeptide complex may specifically bind CD28, CD3, CD19, and CD20. For example, VL1 and VH1 may specifically bind to cMet. For example, VL4 and VH4 may specifically bind to cMet. For example, VL1 and VH1 may specifically bind to Trop2. For example, VL4 and VH4 may specifically bind to Trop2. For example, VL1 and VH1 may specifically bind to CD19. For example, VL4 and VH4 may specifically bind to CD19. For example, VL1 and VH1 may specifically bind to CD20. For example, VL4 and VH4 may specifically bind to CD20.

In some aspects, the invention is directed to an antigen binding polypeptide complex comprising a first polypeptide and a second polypeptide; wherein the first polypeptide has a structure represented by VL1-L1-VL2-L2-VH2-L3-VH1-L4-Fc; or VH1-L5-VH2-L6-VL2-L7-VL1-L8-Fc; wherein the second polypeptide has a structure represented by VL3-L9-VL4-L10-VH4-L11-VH3-L12-Fc; or VH3-L13-VH4-L14-VL4-L15-VL3-L16-Fc; wherein VL1 is a first immunoglobulin light chain variable region that specifically binds to A2AR, APRIL, ATPDase, BAFF, BAFFR, BCMA, BlyS, BTK, BTLA, B7DC, B7H1, B7H2, B7H3, B7H4, B7H5, B7H6, B7H7, B7RP1, B7-4, C3, C5, CCL2, CCL3, CCL4, CCL5, CCL7, CCL8, CCL11, CCL15, CCL17, CCL19, CCL20, CCL21, CCL25, CCR3, CCR4, CD16A, CD19, CD20, CD24, CD27, CD28, CD30, CD38, CD39, CD40, CD40L, CD47, CD52, CD70, CD80, CD86, CD123, CD133, CD137, CD137L, CD160, CD272, CEACAM5, CLEC9, CLEC91, CRTH2, CSF-1, CSF-2, CSF-3, CXCL1, CXCL2, CXCL4, CXCL12, CXCL13, CXCR3, cMet, CTLA4, DLL3, DLL4, DNGR-1, E-cadherin, EGFR, ENTPD1, EpCAM, FCER1, FCER1A, FCER2, FGFR, FLAP, FOLH1, Gi24, GITR, GITRL, GPR5, GP100, GPRC5D, HER2, HER3, ICOSL, ICOS, HHLA2, HMGB1, HVEM, IDO, IFNa, IgE, IGF1R, IL2Rbeta, IL1, ILIA, IL1B, IL1F10, IL2, IL4, IL4Ra, IL5, IL5R, IL6, IL7, IL7Ra, IL8, IL9, IL9R, IL10, rhILlO, IL12, IL13, IL13Ra1, IL13Ra2, IL15, IL17, IL17Rb, IL18, IL22, IL23, IL25, IL7, IL33, IL35, ITGB4, ITK, KIR, LAG3, LAMP1, leptin, LPFS2, MHC class II, MUC-1, MUC-16, NCR3LG1, NKG2D, NKp46, NTPDase-1, OX40, OX40L, PD-1, PD-L1, PD-L2, PROM1, S152, SIRPalpha, SISP1, SLC, SPG64, ST2, STEAP1, STEAP2, Syk kinase, STEAP1, TROP2, TACI, TDO, TGFBETA, T14, TIGIT, TIM3, TLR, TLR2, TLR4, TLR5, TLR9, TMEF1, TNFa, TNFRSF7, Tp55, TREM1, TSLP, TSLPR, TWEAK, VEGF, VISTA, Vstm3, or WUCAM; VL2 is a second immunoglobulin light chain variable region that specifically binds to A2AR, APRIL, ATPDase, BAFF, BAFFR, BCMA, BlyS, BTK, BTLA, B7DC, B7H1, B7H2, B7H3, B7H4, B7H5, B7H6, B7H7, B7RP1, B7-4, C3, C5, CCL2, CCL3, CCL4, CCL5, CCL7, CCL8, CCL11, CCL15, CCL17, CCL19, CCL20, CCL21, CCL25, CCR3, CCR4, CD16A, CD19, CD20, CD24, CD27, CD28, CD30, CD38, CD39, CD40, CD40L, CD47, CD52, CD70, CD80, CD86, CD123, CD133, CD137, CD137L, CD160, CD272, CEACAM5, CLEC9, CLEC91, CRTH2, CSF-1, CSF-2, CSF-3, CXCL1, CXCL2, CXCL4, CXCL12, CXCL13, CXCR3, cMet, CTLA4, DLL3, DLL4, DNGR-1, E-cadherin, EGFR, ENTPD1, EpCAM, FCER1, FCER1A, FCER2, FGFR, FLAP, FOLH1, Gi24, GITR, GITRL, GPR5, GP100, GPRC5D, HER2, HER3, ICOSL, ICOS, HHLA2, HMGB1, HVEM, IDO, IFNa, IgE, IGF1R, IL2Rbeta, IL1, ILIA, IL1B, IL1F10, IL2, IL4, IL4Ra, IL5, IL5R, IL6, IL7, IL7Ra, IL8, IL9, IL9R, IL10, rhILlO, IL12, IL13, IL13Ra1, IL13Ra2, IL15, IL17, IL17Rb, IL18, IL22, IL23, IL25, IL7, IL33, IL35, ITGB4, ITK, KIR, LAG3, LAMP1, leptin, LPFS2, MHC class II, MUC-1, MUC-16, NCR3LG1, NKG2D, NKp46, NTPDase-1, OX40, OX40L, PD-1, PD-L1, PD-L2, PROM1, 5152, SIRPalpha, SISP1, SLC, SPG64, ST2, STEAP1, STEAP2, Syk kinase, STEAP1, TROP2, TACI, TDO, TGFBETA, T14, TIGIT, TIM3, TLR, TLR2, TLR4, TLR5, TLR9, TMEF1, TNFa, TNFRSF7, Tp55, TREM1, TSLP, TSLPR, TWEAK, VEGF, VISTA, Vstm3, or WUCAM; VL3 is a third immunoglobulin light chain variable region that specifically binds to CD3; VL4 is a fourth immunoglobulin light chain variable region that specifically binds to CD28; VH1 is a first immunoglobulin heavy chain variable region that specifically binds to A2AR, APRIL, ATPDase, BAFF, BAFFR, BCMA, BlyS, BTK, BTLA, B7DC, B7H1, B7H2, B7H3, B7H4, B7H5, B7H6, B7H7, B7RP1, B7-4, C3, C5, CCL2, CCL3, CCL4, CCL5, CCL7, CCL8, CCL11, CCL15, CCL17, CCL19, CCL20, CCL21, CCL25, CCR3, CCR4, CD16A, CD19, CD20, CD24, CD27, CD28, CD30, CD38, CD39, CD40, CD40L, CD47, CD52, CD70, CD80, CD86, CD123, CD133, CD137, CD137L, CD160, CD272, CEACAM5, CLEC9, CLEC91, CRTH2, CSF-1, CSF-2, CSF-3, CXCL1, CXCL2, CXCL4, CXCL12, CXCL13, CXCR3, cMet, CTLA4, DLL3, DLL4, DNGR-1, E-cadherin, EGFR, ENTPD1, EpCAM, FCER1, FCER1A, FCER2, FGFR, FLAP, FOLH1, Gi24, GITR, GITRL, GPR5, GP100, GPRC5D, HER2, HER3, ICOSL, ICOS, HHLA2, HMGB1, HVEM, IDO, IFNa, IgE, IGF1R, IL2Rbeta, IL1, ILIA, IL1B, IL1F10, IL2, IL4, IL4Ra, IL5, IL5R, IL6, IL7, IL7Ra, IL8, IL9, IL9R, IL10, rhILlO, IL12, IL13, IL13Ra1, IL13Ra2, IL15, IL17, IL17Rb, IL18, IL22, IL23, IL25, IL7, IL33, IL35, ITGB4, ITK, KIR, LAG3, LAMP1, leptin, LPFS2, MHC class II, MUC-1, MUC-16, NCR3LG1, NKG2D, NKp46, NTPDase-1, OX40, OX40L, PD-1, PD-L1, PD-L2, PROM1, S152, SIRPalpha, SISP1, SLC, SPG64, ST2, STEAP1, STEAP2, Syk kinase, STEAP1, TROP2, TACI, TDO, TGFBETA, T14, TIGIT, TIM3, TLR, TLR2, TLR4, TLR5, TLR9, TMEF1, TNFa, TNFRSF7, Tp55, TREM1, TSLP, TSLPR, TWEAK, VEGF, VISTA, Vstm3, or WUCAM; VH2 is a second immunoglobulin heavy chain variable region that specifically binds to A2AR, APRIL, ATPDase, BAFF, BAFFR, BCMA, BlyS, BTK, BTLA, B7DC, B7H1, B7H2, B7H3, B7H4, B7H5, B7H6, B7H7, B7RP1, B7-4, C3, C5, CCL2, CCL3, CCL4, CCL5, CCL7, CCL8, CCL11, CCL15, CCL17, CCL19, CCL20, CCL21, CCL25, CCR3, CCR4, CD16A, CD19, CD20, CD24, CD27, CD28, CD30, CD38, CD39, CD40, CD40L, CD47, CD52, CD70, CD80, CD86, CD123, CD133, CD137, CD137L, CD160, CD272, CEACAM5, CLEC9, CLEC91, CRTH2, CSF-1, CSF-2, CSF-3, CXCL1, CXCL2, CXCL4, CXCL12, CXCL13, CXCR3, cMet, CTLA4, DLL3, DLL4, DNGR-1, E-cadherin, EGFR, ENTPD1, EpCAM, FCER1, FCER1A, FCER2, FGFR, FLAP, FOLH1, Gi24, GITR, GITRL, GPR5, GP100, GPRC5D, HER2, HER3, ICOSL, ICOS, HHLA2, HMGB1, HVEM, IDO, IFNa, IgE, IGF1R, IL2Rbeta, IL1, ILIA, IL1B, IL1F10, IL2, IL4, IL4Ra, IL5, IL5R, IL6, IL7, IL7Ra, IL8, IL9, IL9R, IL10, rhILlO, IL12, IL13, IL13Ra1, IL13Ra2, IL15, IL17, IL17Rb, IL18, IL22, IL23, IL25, IL7, IL33, IL35, ITGB4, ITK, KIR, LAG3, LAMP1, leptin, LPFS2, MHC class II, MUC-1, MUC-16, NCR3LG1, NKG2D, NKp46, NTPDase-1, OX40, OX40L, PD-1, PD-L1, PD-L2, PROM1, S152, SIRPalpha, SISP1, SLC, SPG64, ST2, STEAP1, STEAP2, Syk kinase, STEAP1, TROP2, TACI, TDO, TGFBETA, T14, TIGIT, TIM3, TLR, TLR2, TLR4, TLR5, TLR9, TMEF1, TNFa, TNFRSF7, Tp55, TREM1, TSLP, TSLPR, TWEAK, VEGF, VISTA, Vstm3, or WUCAM; VH3 is a third immunoglobulin heavy chain variable region that specifically binds to CD3; VH4 is a fourth immunoglobulin heavy chain variable region that specifically binds to CD28; Fc is a region comprising an immunoglobulin heavy chain constant region 2 (CH2), an immunoglobulin heavy chain constant region 3 (CH3), and optionally, an immunoglobulin hinge; and L1-L16 are amino acid linkers. In some aspects, the first polypeptide has a structure represented by VL1-L1-VL2-L2-VH2-L3-VH1-L4-Fc and the second polypeptide has a structure represented by VL3-L9-VL4-L10-VH4-L11-VH3-L12-Fc. In some aspects, the first polypeptide has a structure represented by VL1-L1-VL2-L2-VH2-L3-VH1-L4-Fc and the second polypeptide has a structure represented by VH3-L13-VH4-L14-VL4-L15-VL3-L16-Fc. In some aspects, the first polypeptide has a structure represented by VH1-L5-VH2-L6-VL2-L7-VL1-L8-Fc and the second polypeptide has a structure represented by VL3-L9-VL4-L10-VH4-L11-VH3-L12-Fc. In some aspects, the first polypeptide has a structure represented by VH1-L5-VH2-L6-VL2-L7-VL1-L8-Fc and the second polypeptide has a structure represented by VH3-L13-VH4-L14-VL4-L15-VL3-L16-Fc. In some aspects, VL1 and VH1 may specifically bind to A2AR, APRIL, ATPDase, BAFF, BAFFR, BCMA, BlyS, BTK, BTLA, B7DC, B7H1, B7H2, B7H3, B7H4, B7H5, B7H6, B7H7, B7RP1, B7-4, C3, C5, CCL2, CCL3, CCL4, CCL5, CCL7, CCL8, CCL11, CCL15, CCL17, CCL19, CCL20, CCL21, CCL25, CCR3, CCR4, CD16A, CD19, CD20, CD24, CD27, CD28, CD30, CD38, CD39, CD40, CD40L, CD47, CD52, CD70, CD80, CD86, CD123, CD133, CD137, CD137L, CD160, CD272, CEACAM5, CLEC9, CLEC91, CRTH2, CSF-1, CSF-2, CSF-3, CXCL1, CXCL2, CXCL4, CXCL12, CXCL13, CXCR3, cMet, CTLA4, DLL3, DLL4, DNGR-1, E-cadherin, EGFR, ENTPD1, EpCAM, FCER1, FCER1A, FCER2, FGFR, FLAP, FOLH1, Gi24, GITR, GITRL, GPR5, GP100, GPRC5D, HER2, HER3, ICOSL, ICOS, HHLA2, HMGB1, HVEM, IDO, IFNa, IgE, IGF1R, IL2Rbeta, IL1, ILIA, IL1B, IL1F10, IL2, IL4, IL4Ra, IL5, IL5R, IL6, IL7, IL7Ra, IL8, IL9, IL9R, IL10, rhILlO, IL12, IL13, IL13Ra1, IL13Ra2, IL15, IL17, IL17Rb, IL18, IL22, IL23, IL25, IL7, IL33, IL35, ITGB4, ITK, KIR, LAG3, LAMP1, leptin, LPFS2, MHC class II, MUC-1, MUC-16, NCR3LG1, NKG2D, NKp46, NTPDase-1, OX40, OX40L, PD-1, PD-L1, PD-L2, PROM1, S152, SIRPalpha, SISP1, SLC, SPG64, ST2, STEAP1, STEAP2, Syk kinase, STEAP1, TROP2, TACI, TDO, TGFBETA, T14, TIGIT, TIM3, TLR, TLR2, TLR4, TLR5, TLR9, TMEF1, TNFa, TNFRSF7, Tp55, TREM1, TSLP, TSLPR, TWEAK, VEGF, VISTA, Vstm3, or WUCAM. In some aspects, VL2 and VH2 may specifically bind to A2AR, APRIL, ATPDase, BAFF, BAFFR, BCMA, BlyS, BTK, BTLA, B7DC, B7H1, B7H2, B7H3, B7H4, B7H5, B7H6, B7H7, B7RP1, B7-4, C3, C5, CCL2, CCL3, CCL4, CCL5, CCL7, CCL8, CCL11, CCL15, CCL17, CCL19, CCL20, CCL21, CCL25, CCR3, CCR4, CD16A, CD19, CD20, CD24, CD27, CD28, CD30, CD38, CD39, CD40, CD40L, CD47, CD52, CD70, CD80, CD86, CD123, CD133, CD137, CD137L, CD160, CD272, CEACAM5, CLEC9, CLEC91, CRTH2, CSF-1, CSF-2, CSF-3, CXCL1, CXCL2, CXCL4, CXCL12, CXCL13, CXCR3, cMet, CTLA4, DLL3, DLL4, DNGR-1, E-cadherin, EGFR, ENTPD1, EpCAM, FCER1, FCER1A, FCER2, FGFR, FLAP, FOLH1, Gi24, GITR, GITRL, GPR5, GP100, GPRC5D, HER2, HER3, ICOSL, ICOS, HHLA2, HMGB1, HVEM, IDO, IFNa, IgE, IGF1R, IL2Rbeta, IL1, ILIA, IL1B, IL1F10, IL2, IL4, IL4Ra, IL5, IL5R, IL6, IL7, IL7Ra, IL8, IL9, IL9R, IL10, rhILlO, IL12, IL13, IL13Ra1, IL13Ra2, IL15, IL17, IL17Rb, IL18, IL22, IL23, IL25, IL7, IL33, IL35, ITGB4, ITK, KIR, LAG3, LAMP1, leptin, LPFS2, MHC class II, MUC-1, MUC-16, NCR3LG1, NKG2D, NKp46, NTPDase-1, OX40, OX40L, PD-1, PD-L1, PD-L2, PROM1, S152, SIRPalpha, SISP1, SLC, SPG64, ST2, STEAP1, STEAP2, Syk kinase, STEAP1, TROP2, TACI, TDO, TGFBETA, T14, TIGIT, TIM3, TLR, TLR2, TLR4, TLR5, TLR9, TMEF1, TNFa, TNFRSF7, Tp55, TREM1, TSLP, TSLPR, TWEAK, VEGF, VISTA, Vstm3, or WUCAM. For example, the antigen binding polypeptide complex may specifically bind CD28, CD3, cMet, and Trop2. For example, the antigen binding polypeptide complex may specifically bind CD28, CD3, CD19, and CD20. For example, VL1 and VH1 may specifically bind to cMet. For example, VL2 and VH2 may specifically bind to cMet. For example, VL1 and VH1 may specifically bind to Trop2. For example, VL2 and VH2 may specifically bind to Trop2. For example, VL1 and VH1 may specifically bind to CD19. For example, VL2 and VH2 may specifically bind to CD19. For example, VL1 and VH1 may specifically bind to CD20. For example, VL2 and VH2 may specifically bind to CD20.

In some aspects of the antigen binding polypeptide complexes described herein, VL3 and VH3 specifically bind to CD3, VL4 and VH4 specifically bind to CD28, VL2 and VH2 specifically bind to Trop2, and VL1 and VH1 specifically bind to cMet. In some aspects, VL3 and VH3 specifically bind to CD3, VL4 and VH4 specifically bind to CD28, VL2 and VH2 specifically bind to cMet, and VL1 and VH1 specifically bind to Trop2. In some aspects, VL3 and VH3 specifically bind to CD3, VL4 and VH4 specifically bind to Trop2, VL2 and VH2 specifically bind to CD28, and VL1 and VH1 specifically bind to cMet. In some aspects, VL3 and VH3 specifically bind to CD3, VL4 and VH4 specifically bind to Trop2, VL2 and VH2 specifically bind to cMet, and VL1 and VH1 specifically bind to CD28. In some aspects, VL3 and VH3 specifically bind to CD3, VL4 and VH4 specifically bind to cMet, VL2 and VH2 specifically bind to Trop2, and VL1 and VH1 specifically bind to CD28. In some aspects, VL3 and VH3 specifically bind to CD3, VL4 and VH4 specifically bind to cMet, VL2 and VH2 specifically bind to CD28, and VL1 and VH1 specifically bind to Trop2. In some aspects, VL3 and VH3 specifically bind to CD3, VL4 and VH4 specifically bind to CD28, VL2 and VH2 specifically bind to CD20, and VL1 and VH1 specifically bind to CD19. In some aspects, VL3 and VH3 specifically bind to CD3, VL4 and VH4 specifically bind to CD28, VL2 and VH2 specifically bind to CD19, and VL1 and VH1 specifically bind to CD20. In some aspects, VL3 and VH3 specifically bind to CD3, VL4 and VH4 specifically bind to CD20, VL2 and VH2 specifically bind to CD28, and VL1 and VH1 specifically bind to CD19. In some aspects, VL3 and VH3 specifically bind to CD3, VL4 and VH4 specifically bind to CD20, VL2 and VH2 specifically bind to CD19, and VL1 and VH1 specifically bind to CD28. In some aspects, VL3 and VH3 specifically bind to CD3, VL4 and VH4 specifically bind to CD19, VL2 and VH2 specifically bind to CD20, and VL1 and VH1 specifically bind to CD28. In some aspects, VL3 and VH3 specifically bind to CD3, VL4 and VH4 specifically bind to CD19, VL2 and VH2 specifically bind to CD28, and VL1 and VH1 specifically bind to CD20.

In some aspects, the invention is directed to an antigen binding polypeptide complex comprising a first polypeptide and a second polypeptide; wherein the first polypeptide has a structure represented by VL1-L1-VL2-L2-VH2-L3-VH1-L4-Fc; or VH1-L5-VH2-L6-VL2-L7-VL1-L8-Fc; wherein the second polypeptide has a structure represented by VL3-L9-VL4-L10-VH4-L11-VH3-L12-Fc; or VH3-L13-VH4-L14-VL4-L15-VL3-L16-Fc; wherein VL1 is a first immunoglobulin light chain variable region that specifically binds to A2AR, APRIL, ATPDase, BAFF, BAFFR, BCMA, BlyS, BTK, BTLA, B7DC, B7H1, B7H2, B7H3, B7H4, B7H5, B7H6, B7H7, B7RP1, B7-4, C3, C5, CCL2, CCL3, CCL4, CCL5, CCL7, CCL8, CCL11, CCL15, CCL17, CCL19, CCL20, CCL21, CCL25, CCR3, CCR4, CD16A, CD19, CD20, CD24, CD27, CD28, CD30, CD38, CD39, CD40, CD40L, CD47, CD52, CD70, CD80, CD86, CD123, CD133, CD137, CD137L, CD160, CD272, CEACAM5, CLEC9, CLEC91, CRTH2, CSF-1, CSF-2, CSF-3, CXCL1, CXCL2, CXCL4, CXCL12, CXCL13, CXCR3, cMet, CTLA4, DLL3, DLL4, DNGR-1, E-cadherin, EGFR, ENTPD1, EpCAM, FCER1, FCER1A, FCER2, FGFR, FLAP, FOLH1, Gi24, GITR, GITRL, GPR5, GP100, GPRC5D, HER2, HER3, ICOSL, ICOS, HHLA2, HMGB1, HVEM, IDO, IFNa, IgE, IGF1R, IL2Rbeta, IL1, ILIA, IL1B, IL1F10, IL2, IL4, IL4Ra, IL5, IL5R, IL6, IL7, IL7Ra, IL8, IL9, IL9R, IL10, rhILlO, IL12, IL13, IL13Ra1, IL13Ra2, IL15, IL17, IL17Rb, IL18, IL22, IL23, IL25, IL7, IL33, IL35, ITGB4, ITK, KIR, LAG3, LAMP1, leptin, LPFS2, MHC class II, MUC-1, MUC-16, NCR3LG1, NKG2D, NKp46, NTPDase-1, OX40, OX40L, PD-1, PD-L1, PD-L2, PROM1, S152, SIRPalpha, SISP1, SLC, SPG64, ST2, STEAP1, STEAP2, Syk kinase, STEAP1, TROP2, TACI, TDO, TGFBETA, T14, TIGIT, TIM3, TLR, TLR2, TLR4, TLR5, TLR9, TMEF1, TNFa, TNFRSF7, Tp55, TREM1, TSLP, TSLPR, TWEAK, VEGF, VISTA, Vstm3, or WUCAM; VL2 is a second immunoglobulin light chain variable region that specifically binds to A2AR, APRIL, ATPDase, BAFF, BAFFR, BCMA, BlyS, BTK, BTLA, B7DC, B7H1, B7H2, B7H3, B7H4, B7H5, B7H6, B7H7, B7RP1, B7-4, C3, C5, CCL2, CCL3, CCL4, CCL5, CCL7, CCL8, CCL11, CCL15, CCL17, CCL19, CCL20, CCL21, CCL25, CCR3, CCR4, CD16A, CD19, CD20, CD24, CD27, CD28, CD30, CD38, CD39, CD40, CD40L, CD47, CD52, CD70, CD80, CD86, CD123, CD133, CD137, CD137L, CD160, CD272, CEACAM5, CLEC9, CLEC91, CRTH2, CSF-1, CSF-2, CSF-3, CXCL1, CXCL2, CXCL4, CXCL12, CXCL13, CXCR3, cMet, CTLA4, DLL3, DLL4, DNGR-1, E-cadherin, EGFR, ENTPD1, EpCAM, FCER1, FCER1A, FCER2, FGFR, FLAP, FOLH1, Gi24, GITR, GITRL, GPR5, GP100, GPRC5D, HER2, HER3, ICOSL, ICOS, HHLA2, HMGB1, HVEM, IDO, IFNa, IgE, IGF1R, IL2Rbeta, IL1, ILIA, IL1B, IL1F10, IL2, IL4, IL4Ra, IL5, IL5R, IL6, IL7, IL7Ra, IL8, IL9, IL9R, IL10, rhILlO, IL12, IL13, IL13Ra1, IL13Ra2, IL15, IL17, IL17Rb, IL18, IL22, IL23, IL25, IL7, IL33, IL35, ITGB4, ITK, KIR, LAG3, LAMP1, leptin, LPFS2, MHC class II, MUC-1, MUC-16, NCR3LG1, NKG2D, NKp46, NTPDase-1, OX40, OX40L, PD-1, PD-L1, PD-L2, PROM1, S152, SIRPalpha, SISP1, SLC, SPG64, ST2, STEAP1, STEAP2, Syk kinase, STEAP1, TROP2, TACI, TDO, TGFBETA, T14, TIGIT, TIM3, TLR, TLR2, TLR4, TLR5, TLR9, TMEF1, TNFa, TNFRSF7, Tp55, TREM1, TSLP, TSLPR, TWEAK, VEGF, VISTA, Vstm3, or WUCAM; VL3 is a third immunoglobulin light chain variable region that specifically binds to A2AR, APRIL, ATPDase, BAFF, BAFFR, BCMA, BlyS, BTK, BTLA, B7DC, B7H1, B7H2, B7H3, B7H4, B7H5, B7H6, B7H7, B7RP1, B7-4, C3, C5, CCL2, CCL3, CCL4, CCL5, CCL7, CCL8, CCL11, CCL15, CCL17, CCL19, CCL20, CCL21, CCL25, CCR3, CCR4, CD16A, CD19, CD20, CD24, CD27, CD28, CD30, CD38, CD39, CD40, CD40L, CD47, CD52, CD70, CD80, CD86, CD123, CD133, CD137, CD137L, CD160, CD272, CEACAM5, CLEC9, CLEC91, CRTH2, CSF-1, CSF-2, CSF-3, CXCL1, CXCL2, CXCL4, CXCL12, CXCL13, CXCR3, cMet, CTLA4, DLL3, DLL4, DNGR-1, E-cadherin, EGFR, ENTPD1, EpCAM, FCER1, FCER1A, FCER2, FGFR, FLAP, FOLH1, Gi24, GITR, GITRL, GPR5, GP100, GPRC5D, HER2, HER3, ICOSL, ICOS, HHLA2, HMGB1, HVEM, IDO, IFNa, IgE, IGF1R, IL2Rbeta, IL1, ILIA, IL1B, IL1F10, IL2, IL4, IL4Ra, IL5, IL5R, IL6, IL7, IL7Ra, IL8, IL9, IL9R, IL10, rhILlO, IL12, IL13, IL13Ra1, IL13Ra2, IL15, IL17, IL17Rb, IL18, IL22, IL23, IL25, IL7, IL33, IL35, ITGB4, ITK, KIR, LAG3, LAMP1, leptin, LPFS2, MHC class II, MUC-1, MUC-16, NCR3LG1, NKG2D, NKp46, NTPDase-1, OX40, OX40L, PD-1, PD-L1, PD-L2, PROM1, S152, SIRPalpha, SISP1, SLC, SPG64, ST2, STEAP1, STEAP2, Syk kinase, STEAP1, TROP2, TACI, TDO, TGFBETA, T14, TIGIT, TIM3, TLR, TLR2, TLR4, TLR5, TLR9, TMEF1, TNFa, TNFRSF7, Tp55, TREM1, TSLP, TSLPR, TWEAK, VEGF, VISTA, Vstm3, or WUCAM; VL4 is a fourth immunoglobulin light chain variable region that specifically binds to CD3; VH1 is a first immunoglobulin heavy chain variable region that specifically binds to A2AR, APRIL, ATPDase, BAFF, BAFFR, BCMA, BlyS, BTK, BTLA, B7DC, B7H1, B7H2, B7H3, B7H4, B7H5, B7H6, B7H7, B7RP1, B7-4, C3, C5, CCL2, CCL3, CCL4, CCL5, CCL7, CCL8, CCL11, CCL15, CCL17, CCL19, CCL20, CCL21, CCL25, CCR3, CCR4, CD16A, CD19, CD20, CD24, CD27, CD28, CD30, CD38, CD39, CD40, CD40L, CD47, CD52, CD70, CD80, CD86, CD123, CD133, CD137, CD137L, CD160, CD272, CEACAM5, CLEC9, CLEC91, CRTH2, CSF-1, CSF-2, CSF-3, CXCL1, CXCL2, CXCL4, CXCL12, CXCL13, CXCR3, cMet, CTLA4, DLL3, DLL4, DNGR-1, E-cadherin, EGFR, ENTPD1, EpCAM, FCER1, FCER1A, FCER2, FGFR, FLAP, FOLH1, Gi24, GITR, GITRL, GPR5, GP100, GPRC5D, HER2, HER3, ICOSL, ICOS, HHLA2, HMGB1, HVEM, IDO, IFNa, IgE, IGF1R, IL2Rbeta, IL1, ILIA, IL1B, IL1F10, IL2, IL4, IL4Ra, IL5, IL5R, IL6, IL7, IL7Ra, IL8, IL9, IL9R, IL10, rhILlO, IL12, IL13, IL13Ra1, IL13Ra2, IL15, IL17, IL17Rb, IL18, IL22, IL23, IL25, IL7, IL33, IL35, ITGB4, ITK, KIR, LAG3, LAMP1, leptin, LPFS2, MHC class II, MUC-1, MUC-16, NCR3LG1, NKG2D, NKp46, NTPDase-1, OX40, OX40L, PD-1, PD-L1, PD-L2, PROM1, 5152, SIRPalpha, SISP1, SLC, SPG64, ST2, STEAP1, STEAP2, Syk kinase, STEAP1, TROP2, TACI, TDO, TGFBETA, T14, TIGIT, TIM3, TLR, TLR2, TLR4, TLR5, TLR9, TMEF1, TNFa, TNFRSF7, Tp55, TREM1, TSLP, TSLPR, TWEAK, VEGF, VISTA, Vstm3, or WUCAM; VH2 is a second immunoglobulin heavy chain variable region that specifically binds to A2AR, APRIL, ATPDase, BAFF, BAFFR, BCMA, BlyS, BTK, BTLA, B7DC, B7H1, B7H2, B7H3, B7H4, B7H5, B7H6, B7H7, B7RP1, B7-4, C3, C5, CCL2, CCL3, CCL4, CCL5, CCL7, CCL8, CCL11, CCL15, CCL17, CCL19, CCL20, CCL21, CCL25, CCR3, CCR4, CD16A, CD19, CD20, CD24, CD27, CD28, CD30, CD38, CD39, CD40, CD40L, CD47, CD52, CD70, CD80, CD86, CD123, CD133, CD137, CD137L, CD160, CD272, CEACAM5, CLEC9, CLEC91, CRTH2, CSF-1, CSF-2, CSF-3, CXCL1, CXCL2, CXCL4, CXCL12, CXCL13, CXCR3, cMet, CTLA4, DLL3, DLL4, DNGR-1, E-cadherin, EGFR, ENTPD1, EpCAM, FCER1, FCER1A, FCER2, FGFR, FLAP, FOLH1, Gi24, GITR, GITRL, GPR5, GP100, GPRC5D, HER2, HER3, ICOSL, ICOS, HHLA2, HMGB1, HVEM, IDO, IFNa, IgE, IGF1R, IL2Rbeta, IL1, ILIA, IL1B, IL1F10, IL2, IL4, IL4Ra, IL5, IL5R, IL6, IL7, IL7Ra, IL8, IL9, IL9R, IL10, rhILlO, IL12, IL13, IL13Ra1, IL13Ra2, IL15, IL17, IL17Rb, IL18, IL22, IL23, IL25, IL7, IL33, IL35, ITGB4, ITK, KIR, LAG3, LAMP1, leptin, LPFS2, MHC class II, MUC-1, MUC-16, NCR3LG1, NKG2D, NKp46, NTPDase-1, OX40, OX40L, PD-1, PD-L1, PD-L2, PROM1, S152, SIRPalpha, SISP1, SLC, SPG64, ST2, STEAP1, STEAP2, Syk kinase, STEAP1, TROP2, TACI, TDO, TGFBETA, T14, TIGIT, TIM3, TLR, TLR2, TLR4, TLR5, TLR9, TMEF1, TNFa, TNFRSF7, Tp55, TREM1, TSLP, TSLPR, TWEAK, VEGF, VISTA, Vstm3, or WUCAM; VH3 is a third immunoglobulin heavy chain variable region that specifically binds to A2AR, APRIL, ATPDase, BAFF, BAFFR, BCMA, BlyS, BTK, BTLA, B7DC, B7H1, B7H2, B7H3, B7H4, B7H5, B7H6, B7H7, B7RP1, B7-4, C3, C5, CCL2, CCL3, CCL4, CCL5, CCL7, CCL8, CCL11, CCL15, CCL17, CCL19, CCL20, CCL21, CCL25, CCR3, CCR4, CD16A, CD19, CD20, CD24, CD27, CD28, CD30, CD38, CD39, CD40, CD40L, CD47, CD52, CD70, CD80, CD86, CD123, CD133, CD137, CD137L, CD160, CD272, CEACAM5, CLEC9, CLEC91, CRTH2, CSF-1, CSF-2, CSF-3, CXCL1, CXCL2, CXCL4, CXCL12, CXCL13, CXCR3, cMet, CTLA4, DLL3, DLL4, DNGR-1, E-cadherin, EGFR, ENTPD1, EpCAM, FCER1, FCER1A, FCER2, FGFR, FLAP, FOLH1, Gi24, GITR, GITRL, GPR5, GP100, GPRC5D, HER2, HER3, ICOSL, ICOS, HHLA2, HMGB1, HVEM, IDO, IFNa, IgE, IGF1R, IL2Rbeta, IL1, ILIA, IL1B, IL1F10, IL2, IL4, IL4Ra, IL5, IL5R, IL6, IL7, IL7Ra, IL8, IL9, IL9R, IL10, rhILlO, IL12, IL13, IL13Ra1, IL13Ra2, IL15, IL17, IL17Rb, IL18, IL22, IL23, IL25, IL7, IL33, IL35, ITGB4, ITK, KIR, LAG3, LAMP1, leptin, LPFS2, MHC class II, MUC-1, MUC-16, NCR3LG1, NKG2D, NKp46, NTPDase-1, OX40, OX40L, PD-1, PD-L1, PD-L2, PROM1, S152, SIRPalpha, SISP1, SLC, SPG64, ST2, STEAP1, STEAP2, Syk kinase, STEAP1, TROP2, TACI, TDO, TGFBETA, T14, TIGIT, TIM3, TLR, TLR2, TLR4, TLR5, TLR9, TMEF1, TNFa, TNFRSF7, Tp55, TREM1, TSLP, TSLPR, TWEAK, VEGF, VISTA, Vstm3, or WUCAM; VH4 is a fourth immunoglobulin heavy chain variable region that specifically binds to CD3; Fe is a region comprising an immunoglobulin heavy chain constant region 2 (CH2), an immunoglobulin heavy chain constant region 3 (CH3), and optionally, an immunoglobulin hinge; and L1-L16 are amino acid linkers. In some aspects, the first polypeptide has a structure represented by VL1-L1-VL2-L2-VH2-L3-VH1-L4-Fc and the second polypeptide has a structure represented by VL3-L9-VL4-L10-VH4-L11-VH3-L12-Fc. In some aspects, the first polypeptide has a structure represented by VL1-L1-VL2-L2-VH2-L3-VH1-L4-Fc and the second polypeptide has a structure represented by VH3-L13-VH4-L14-VL4-L15-VL3-L16-Fc. In some aspects, the first polypeptide has a structure represented by VH1-L5-VH2-L6-VL2-L7-VL1-L8-Fc and the second polypeptide has a structure represented by VL3-L9-VL4-L10-VH4-L11-VH3-L12-Fc. In some aspects, the first polypeptide has a structure represented by VH1-L5-VH2-L6-VL2-L7-VL1-L8-Fc and the second polypeptide has a structure represented by VH3-L13-VH4-L14-VL4-L15-VL3-L16-Fc. In some aspects, VL1 and VH1 may specifically bind to A2AR, APRIL, ATPDase, BAFF, BAFFR, BCMA, BlyS, BTK, BTLA, B7DC, B7H1, B7H2, B7H3, B7H4, B7H5, B7H6, B7H7, B7RP1, B7-4, C3, C5, CCL2, CCL3, CCL4, CCL5, CCL7, CCL8, CCL11, CCL15, CCL17, CCL19, CCL20, CCL21, CCL25, CCR3, CCR4, CD16A, CD19, CD20, CD24, CD27, CD28, CD30, CD38, CD39, CD40, CD40L, CD47, CD52, CD70, CD80, CD86, CD123, CD133, CD137, CD137L, CD160, CD272, CEACAM5, CLEC9, CLEC91, CRTH2, CSF-1, CSF-2, CSF-3, CXCL1, CXCL2, CXCL4, CXCL12, CXCL13, CXCR3, cMet, CTLA4, DLL3, DLL4, DNGR-1, E-cadherin, EGFR, ENTPD1, EpCAM, FCER1, FCER1A, FCER2, FGFR, FLAP, FOLH1, Gi24, GITR, GITRL, GPR5, GP100, GPRC5D, HER2, HER3, ICOSL, ICOS, HHLA2, HMGB1, HVEM, IDO, IFNa, IgE, IGF1R, IL2Rbeta, IL1, ILIA, IL1B, IL1F10, IL2, IL4, IL4Ra, IL5, IL5R, IL6, IL7, IL7Ra, IL8, IL9, IL9R, IL10, rhILlO, IL12, IL13, IL13Ra1, IL13Ra2, IL15, IL17, IL17Rb, IL18, IL22, IL23, IL25, IL7, IL33, IL35, ITGB4, ITK, KIR, LAG3, LAMP1, leptin, LPFS2, MHC class II, MUC-1, MUC-16, NCR3LG1, NKG2D, NKp46, NTPDase-1, OX40, OX40L, PD-1, PD-L1, PD-L2, PROM1, S152, SIRPalpha, SISP1, SLC, SPG64, ST2, STEAP1, STEAP2, Syk kinase, STEAP1, TROP2, TACI, TDO, TGFBETA, T14, TIGIT, TIM3, TLR, TLR2, TLR4, TLR5, TLR9, TMEF1, TNFa, TNFRSF7, Tp55, TREM1, TSLP, TSLPR, TWEAK, VEGF, VISTA, Vstm3, or WUCAM. In some aspects, VL2 and VH2 may specifically bind to A2AR, APRIL, ATPDase, BAFF, BAFFR, BCMA, BlyS, BTK, BTLA, B7DC, B7H1, B7H2, B7H3, B7H4, B7H5, B7H6, B7H7, B7RP1, B7-4, C3, C5, CCL2, CCL3, CCL4, CCL5, CCL7, CCL8, CCL11, CCL15, CCL17, CCL19, CCL20, CCL21, CCL25, CCR3, CCR4, CD16A, CD19, CD20, CD24, CD27, CD28, CD30, CD38, CD39, CD40, CD40L, CD47, CD52, CD70, CD80, CD86, CD123, CD133, CD137, CD137L, CD160, CD272, CEACAM5, CLEC9, CLEC91, CRTH2, CSF-1, CSF-2, CSF-3, CXCL1, CXCL2, CXCL4, CXCL12, CXCL13, CXCR3, cMet, CTLA4, DLL3, DLL4, DNGR-1, E-cadherin, EGFR, ENTPD1, EpCAM, FCER1, FCER1A, FCER2, FGFR, FLAP, FOLH1, Gi24, GITR, GITRL, GPR5, GP100, GPRC5D, HER2, HER3, ICOSL, ICOS, HHLA2, HMGB1, HVEM, IDO, IFNa, IgE, IGF1R, IL2Rbeta, IL1, ILIA, IL1B, IL1F10, IL2, IL4, IL4Ra, IL5, IL5R, IL6, IL7, IL7Ra, IL8, IL9, IL9R, IL10, rhILlO, IL12, IL13, IL13Ra1, IL13Ra2, IL15, IL17, IL17Rb, IL18, IL22, IL23, IL25, IL7, IL33, IL35, ITGB4, ITK, KIR, LAG3, LAMP1, leptin, LPFS2, MHC class II, MUC-1, MUC-16, NCR3LG1, NKG2D, NKp46, NTPDase-1, OX40, OX40L, PD-1, PD-L1, PD-L2, PROM1, S152, SIRPalpha, SISP1, SLC, SPG64, ST2, STEAP1, STEAP2, Syk kinase, STEAP1, TROP2, TACI, TDO, TGFBETA, T14, TIGIT, TIM3, TLR, TLR2, TLR4, TLR5, TLR9, TMEF1, TNFa, TNFRSF7, Tp55, TREM1, TSLP, TSLPR, TWEAK, VEGF, VISTA, Vstm3, or WUCAM. In some aspects, VL3 and VH3 may specifically bind to A2AR, APRIL, ATPDase, BAFF, BAFFR, BCMA, BlyS, BTK, BTLA, B7DC, B7H1, B7H2, B7H3, B7H4, B7H5, B7H6, B7H7, B7RP1, B7-4, C3, C5, CCL2, CCL3, CCL4, CCL5, CCL7, CCL8, CCL11, CCL15, CCL17, CCL19, CCL20, CCL21, CCL25, CCR3, CCR4, CD16A, CD19, CD20, CD24, CD27, CD28, CD30, CD38, CD39, CD40, CD40L, CD47, CD52, CD70, CD80, CD86, CD123, CD133, CD137, CD137L, CD160, CD272, CEACAM5, CLEC9, CLEC91, CRTH2, CSF-1, CSF-2, CSF-3, CXCL1, CXCL2, CXCL4, CXCL12, CXCL13, CXCR3, cMet, CTLA4, DLL3, DLL4, DNGR-1, E-cadherin, EGFR, ENTPD1, EpCAM, FCER1, FCER1A, FCER2, FGFR, FLAP, FOLH1, Gi24, GITR, GITRL, GPR5, GP100, GPRC5D, HER2, HER3, ICOSL, ICOS, HHLA2, HMGB1, HVEM, IDO, IFNa, IgE, IGF1R, IL2Rbeta, IL1, ILIA, IL1B, IL1F10, IL2, IL4, IL4Ra, IL5, IL5R, IL6, IL7, IL7Ra, IL8, IL9, IL9R, IL10, rhILlO, IL12, IL13, IL13Ra1, IL13Ra2, IL15, IL17, IL17Rb, IL18, IL22, IL23, IL25, IL7, IL33, IL35, ITGB4, ITK, KIR, LAG3, LAMP1, leptin, LPFS2, MHC class II, MUC-1, MUC-16, NCR3LG1, NKG2D, NKp46, NTPDase-1, OX40, OX40L, PD-1, PD-L1, PD-L2, PROM1, S152, SIRPalpha, SISP1, SLC, SPG64, ST2, STEAP1, STEAP2, Syk kinase, STEAP1, TROP2, TACI, TDO, TGFBETA, T14, TIGIT, TIM3, TLR, TLR2, TLR4, TLR5, TLR9, TMEF1, TNFa, TNFRSF7, Tp55, TREM1, TSLP, TSLPR, TWEAK, VEGF, VISTA, Vstm3, or WUCAM. For example, the antigen binding polypeptide complex may specifically bind CD28, CD3, cMet, and Trop2. For example, the antigen binding polypeptide complex may specifically bind CD28, CD3, CD19, and CD20. For example, VL1 and VH1 may specifically bind to CD28. For example, VL2 and VH2 may specifically bind to CD28. For example, VL3 and VH3 may specifically bind to CD28. For example, VL1 and VH1 may specifically bind to cMet. For example, VL2 and VH2 may specifically bind to cMet. For example, VL3 and VH3 may specifically bind to cMet. For example, VL1 and VH1 may specifically bind to Trop2. For example, VL2 and VH2 may specifically bind to Trop2. For example, VL3 and VH3 may specifically bind to Trop2. For example, VL1 and VH1 may specifically bind to CD19. For example, VL2 and VH2 may specifically bind to CD19. For example, VL3 and VH3 may specifically bind to CD19. For example, VL1 and VH1 may specifically bind to CD20. For example, VL2 and VH2 may specifically bind to CD20. For example, VL3 and VH3 may specifically bind to CD20.

In some aspects, the invention is directed to an antigen binding polypeptide complex comprising a first polypeptide and a second polypeptide; wherein the first polypeptide has a structure represented by VL1-L1-VL2-L2-VH2-L3-VH1-L4-Fc; or VH1-L5-VH2-L6-VL2-L7-VL1-L8-Fc; wherein the second polypeptide has a structure represented by VL3-L9-VL4-L10-VH4-L11-VH3-L12-Fc; or VH3-L13-VH4-L14-VL4-L15-VL3-L16-Fc; wherein VL1 is a first immunoglobulin light chain variable region that specifically binds to CD28; VL2 is a second immunoglobulin light chain variable region that specifically binds to A2AR, APRIL, ATPDase, BAFF, BAFFR, BCMA, BlyS, BTK, BTLA, B7DC, B7H1, B7H2, B7H3, B7H4, B7H5, B7H6, B7H7, B7RP1, B7-4, C3, C5, CCL2, CCL3, CCL4, CCL5, CCL7, CCL8, CCL11, CCL15, CCL17, CCL19, CCL20, CCL21, CCL25, CCR3, CCR4, CD16A, CD19, CD20, CD24, CD27, CD28, CD30, CD38, CD39, CD40, CD40L, CD47, CD52, CD70, CD80, CD86, CD123, CD133, CD137, CD137L, CD160, CD272, CEACAM5, CLEC9, CLEC91, CRTH2, CSF-1, CSF-2, CSF-3, CXCL1, CXCL2, CXCL4, CXCL12, CXCL13, CXCR3, cMet, CTLA4, DLL3, DLL4, DNGR-1, E-cadherin, EGFR, ENTPD1, EpCAM, FCER1, FCER1A, FCER2, FGFR, FLAP, FOLH1, Gi24, GITR, GITRL, GPR5, GP100, GPRC5D, HER2, HER3, ICOSL, ICOS, HHLA2, HMGB1, HVEM, IDO, IFNa, IgE, IGF1R, IL2Rbeta, IL1, ILIA, IL1B, IL1F10, IL2, IL4, IL4Ra, IL5, IL5R, IL6, IL7, IL7Ra, IL8, IL9, IL9R, IL10, rhILlO, IL12, IL13, IL13Ra1, IL13Ra2, IL15, IL17, IL17Rb, IL18, IL22, IL23, IL25, IL7, IL33, IL35, ITGB4, ITK, KIR, LAG3, LAMP1, leptin, LPFS2, MHC class II, MUC-1, MUC-16, NCR3LG1, NKG2D, NKp46, NTPDase-1, OX40, OX40L, PD-1, PD-L1, PD-L2, PROM1, 5152, SIRPalpha, SISP1, SLC, SPG64, ST2, STEAP1, STEAP2, Syk kinase, STEAP1, TROP2, TACI, TDO, TGFBETA, T14, TIGIT, TIM3, TLR, TLR2, TLR4, TLR5, TLR9, TMEF1, TNFa, TNFRSF7, Tp55, TREM1, TSLP, TSLPR, TWEAK, VEGF, VISTA, Vstm3, or WUCAM; VL3 is a third immunoglobulin light chain variable region that specifically binds to A2AR, APRIL, ATPDase, BAFF, BAFFR, BCMA, BlyS, BTK, BTLA, B7DC, B7H1, B7H2, B7H3, B7H4, B7H5, B7H6, B7H7, B7RP1, B7-4, C3, C5, CCL2, CCL3, CCL4, CCL5, CCL7, CCL8, CCL11, CCL15, CCL17, CCL19, CCL20, CCL21, CCL25, CCR3, CCR4, CD16A, CD19, CD20, CD24, CD27, CD28, CD30, CD38, CD39, CD40, CD40L, CD47, CD52, CD70, CD80, CD86, CD123, CD133, CD137, CD137L, CD160, CD272, CEACAM5, CLEC9, CLEC91, CRTH2, CSF-1, CSF-2, CSF-3, CXCL1, CXCL2, CXCL4, CXCL12, CXCL13, CXCR3, cMet, CTLA4, DLL3, DLL4, DNGR-1, E-cadherin, EGFR, ENTPD1, EpCAM, FCER1, FCER1A, FCER2, FGFR, FLAP, FOLH1, Gi24, GITR, GITRL, GPR5, GP100, GPRC5D, HER2, HER3, ICOSL, ICOS, HHLA2, HMGB1, HVEM, IDO, IFNa, IgE, IGF1R, IL2Rbeta, IL1, ILIA, IL1B, IL1F10, IL2, IL4, IL4Ra, IL5, IL5R, IL6, IL7, IL7Ra, IL8, IL9, IL9R, IL10, rhILlO, IL12, IL13, IL13Ra1, IL13Ra2, IL15, IL17, IL17Rb, IL18, IL22, IL23, IL25, IL7, IL33, IL35, ITGB4, ITK, KIR, LAG3, LAMP1, leptin, LPFS2, MHC class II, MUC-1, MUC-16, NCR3LG1, NKG2D, NKp46, NTPDase-1, OX40, OX40L, PD-1, PD-L1, PD-L2, PROM1, S152, SIRPalpha, SISP1, SLC, SPG64, ST2, STEAP1, STEAP2, Syk kinase, STEAP1, TROP2, TACI, TDO, TGFBETA, T14, TIGIT, TIM3, TLR, TLR2, TLR4, TLR5, TLR9, TMEF1, TNFa, TNFRSF7, Tp55, TREM1, TSLP, TSLPR, TWEAK, VEGF, VISTA, Vstm3, or WUCAM; VL4 is a fourth immunoglobulin light chain variable region that specifically binds to CD3; VH1 is a first immunoglobulin heavy chain variable region that specifically binds to CD28; VH2 is a second immunoglobulin heavy chain variable region that specifically binds to A2AR, APRIL, ATPDase, BAFF, BAFFR, BCMA, BlyS, BTK, BTLA, B7DC, B7H1, B7H2, B7H3, B7H4, B7H5, B7H6, B7H7, B7RP1, B7-4, C3, C5, CCL2, CCL3, CCL4, CCL5, CCL7, CCL8, CCL11, CCL15, CCL17, CCL19, CCL20, CCL21, CCL25, CCR3, CCR4, CD16A, CD19, CD20, CD24, CD27, CD28, CD30, CD38, CD39, CD40, CD40L, CD47, CD52, CD70, CD80, CD86, CD123, CD133, CD137, CD137L, CD160, CD272, CEACAM5, CLEC9, CLEC91, CRTH2, CSF-1, CSF-2, CSF-3, CXCL1, CXCL2, CXCL4, CXCL12, CXCL13, CXCR3, cMet, CTLA4, DLL3, DLL4, DNGR-1, E-cadherin, EGFR, ENTPD1, EpCAM, FCER1, FCER1A, FCER2, FGFR, FLAP, FOLH1, Gi24, GITR, GITRL, GPR5, GP100, GPRC5D, HER2, HER3, ICOSL, ICOS, HHLA2, HMGB1, HVEM, IDO, IFNa, IgE, IGF1R, IL2Rbeta, IL1, ILIA, IL1B, IL1F10, IL2, IL4, IL4Ra, IL5, IL5R, IL6, IL7, IL7Ra, IL8, IL9, IL9R, IL10, rhILlO, IL12, IL13, IL13Ra1, IL13Ra2, IL15, IL17, IL17Rb, IL18, IL22, IL23, IL25, IL7, IL33, IL35, ITGB4, ITK, KIR, LAG3, LAMP1, leptin, LPFS2, MHC class II, MUC-1, MUC-16, NCR3LG1, NKG2D, NKp46, NTPDase-1, OX40, OX40L, PD-1, PD-L1, PD-L2, PROM1, S152, SIRPalpha, SISP1, SLC, SPG64, ST2, STEAP1, STEAP2, Syk kinase, STEAP1, TROP2, TACI, TDO, TGFBETA, T14, TIGIT, TIM3, TLR, TLR2, TLR4, TLR5, TLR9, TMEF1, TNFa, TNFRSF7, Tp55, TREM1, TSLP, TSLPR, TWEAK, VEGF, VISTA, Vstm3, or WUCAM; VH3 is a third immunoglobulin heavy chain variable region that specifically binds to A2AR, APRIL, ATPDase, BAFF, BAFFR, BCMA, BlyS, BTK, BTLA, B7DC, B7H1, B7H2, B7H3, B7H4, B7H5, B7H6, B7H7, B7RP1, B7-4, C3, C5, CCL2, CCL3, CCL4, CCL5, CCL7, CCL8, CCL11, CCL15, CCL17, CCL19, CCL20, CCL21, CCL25, CCR3, CCR4, CD16A, CD19, CD20, CD24, CD27, CD28, CD30, CD38, CD39, CD40, CD40L, CD47, CD52, CD70, CD80, CD86, CD123, CD133, CD137, CD137L, CD160, CD272, CEACAM5, CLEC9, CLEC91, CRTH2, CSF-1, CSF-2, CSF-3, CXCL1, CXCL2, CXCL4, CXCL12, CXCL13, CXCR3, cMet, CTLA4, DLL3, DLL4, DNGR-1, E-cadherin, EGFR, ENTPD1, EpCAM, FCER1, FCER1A, FCER2, FGFR, FLAP, FOLH1, Gi24, GITR, GITRL, GPR5, GP100, GPRC5D, HER2, HER3, ICOSL, ICOS, HHLA2, HMGB1, HVEM, IDO, IFNa, IgE, IGF1R, IL2Rbeta, IL1, ILIA, IL1B, IL1F10, IL2, IL4, IL4Ra, IL5, IL5R, IL6, IL7, IL7Ra, IL8, IL9, IL9R, IL10, rhILlO, IL12, IL13, IL13Ra1, IL13Ra2, IL15, IL17, IL17Rb, IL18, IL22, IL23, IL25, IL7, IL33, IL35, ITGB4, ITK, KIR, LAG3, LAMP1, leptin, LPFS2, MHC class II, MUC-1, MUC-16, NCR3LG1, NKG2D, NKp46, NTPDase-1, OX40, OX40L, PD-1, PD-L1, PD-L2, PROM1, S152, SIRPalpha, SISP1, SLC, SPG64, ST2, STEAP1, STEAP2, Syk kinase, STEAP1, TROP2, TACI, TDO, TGFBETA, T14, TIGIT, TIM3, TLR, TLR2, TLR4, TLR5, TLR9, TMEF1, TNFa, TNFRSF7, Tp55, TREM1, TSLP, TSLPR, TWEAK, VEGF, VISTA, Vstm3, or WUCAM; VH4 is a fourth immunoglobulin heavy chain variable region that specifically binds to CD3; Fc is a region comprising an immunoglobulin heavy chain constant region 2 (CH2), an immunoglobulin heavy chain constant region 3 (CH3), and optionally, an immunoglobulin hinge; and L1-L16 are amino acid linkers. In some aspects, the first polypeptide has a structure represented by VL1-L1-VL2-L2-VH2-L3-VH1-L4-Fc and the second polypeptide has a structure represented by VL3-L9-VL4-L10-VH4-L11-VH3-L12-Fc. In some aspects, the first polypeptide has a structure represented by VL1-L1-VL2-L2-VH2-L3-VH1-L4-Fc and the second polypeptide has a structure represented by VH3-L13-VH4-L14-VL4-L15-VL3-L16-Fc. In some aspects, the first polypeptide has a structure represented by VH1-L5-VH2-L6-VL2-L7-VL1-L8-Fc and the second polypeptide has a structure represented by VL3-L9-VL4-L10-VH4-L11-VH3-L12-Fc. In some aspects, the first polypeptide has a structure represented by VH1-L5-VH2-L6-VL2-L7-VL1-L8-Fc and the second polypeptide has a structure represented by VH3-L13-VH4-L14-VL4-L15-VL3-L16-Fc. In some aspects, VL2 and VH2 may specifically bind to A2AR, APRIL, ATPDase, BAFF, BAFFR, BCMA, BlyS, BTK, BTLA, B7DC, B7H1, B7H2, B7H3, B7H4, B7H5, B7H6, B7H7, B7RP1, B7-4, C3, C5, CCL2, CCL3, CCL4, CCL5, CCL7, CCL8, CCL11, CCL15, CCL17, CCL19, CCL20, CCL21, CCL25, CCR3, CCR4, CD16A, CD19, CD20, CD24, CD27, CD28, CD30, CD38, CD39, CD40, CD40L, CD47, CD52, CD70, CD80, CD86, CD123, CD133, CD137, CD137L, CD160, CD272, CEACAM5, CLEC9, CLEC91, CRTH2, CSF-1, CSF-2, CSF-3, CXCL1, CXCL2, CXCL4, CXCL12, CXCL13, CXCR3, cMet, CTLA4, DLL3, DLL4, DNGR-1, E-cadherin, EGFR, ENTPD1, EpCAM, FCER1, FCER1A, FCER2, FGFR, FLAP, FOLH1, Gi24, GITR, GITRL, GPR5, GP100, GPRC5D, HER2, HER3, ICOSL, ICOS, HHLA2, HMGB1, HVEM, IDO, IFNa, IgE, IGF1R, IL2Rbeta, IL1, ILIA, IL1B, IL1F10, IL2, IL4, IL4Ra, IL5, IL5R, IL6, IL7, IL7Ra, IL8, IL9, IL9R, IL10, rhILlO, IL12, IL13, IL13Ra1, IL13Ra2, IL15, IL17, IL17Rb, IL18, IL22, IL23, IL25, IL7, IL33, IL35, ITGB4, ITK, KIR, LAG3, LAMP1, leptin, LPFS2, MHC class II, MUC-1, MUC-16, NCR3LG1, NKG2D, NKp46, NTPDase-1, OX40, OX40L, PD-1, PD-L1, PD-L2, PROM1, S152, SIRPalpha, SISP1, SLC, SPG64, ST2, STEAP1, STEAP2, Syk kinase, STEAP1, TROP2, TACI, TDO, TGFBETA, T14, TIGIT, TIM3, TLR, TLR2, TLR4, TLR5, TLR9, TMEF1, TNFa, TNFRSF7, Tp55, TREM1, TSLP, TSLPR, TWEAK, VEGF, VISTA, Vstm3, or WUCAM. In some aspects, VL3 and VH3 may specifically bind to A2AR, APRIL, ATPDase, BAFF, BAFFR, BCMA, BlyS, BTK, BTLA, B7DC, B7H1, B7H2, B7H3, B7H4, B7H5, B7H6, B7H7, B7RP1, B7-4, C3, C5, CCL2, CCL3, CCL4, CCL5, CCL7, CCL8, CCL11, CCL15, CCL17, CCL19, CCL20, CCL21, CCL25, CCR3, CCR4, CD16A, CD19, CD20, CD24, CD27, CD28, CD30, CD38, CD39, CD40, CD40L, CD47, CD52, CD70, CD80, CD86, CD123, CD133, CD137, CD137L, CD160, CD272, CEACAM5, CLEC9, CLEC91, CRTH2, CSF-1, CSF-2, CSF-3, CXCL1, CXCL2, CXCL4, CXCL12, CXCL13, CXCR3, cMet, CTLA4, DLL3, DLL4, DNGR-1, E-cadherin, EGFR, ENTPD1, EpCAM, FCER1, FCER1A, FCER2, FGFR, FLAP, FOLH1, Gi24, GITR, GITRL, GPR5, GP100, GPRC5D, HER2, HER3, ICOSL, ICOS, HHLA2, HMGB1, HVEM, IDO, IFNa, IgE, IGF1R, IL2Rbeta, IL1, ILIA, IL1B, IL1F10, IL2, IL4, IL4Ra, IL5, IL5R, IL6, IL7, IL7Ra, IL8, IL9, IL9R, IL10, rhILlO, IL12, IL13, IL13Ra1, IL13Ra2, IL15, IL17, IL17Rb, IL18, IL22, IL23, IL25, IL7, IL33, IL35, ITGB4, ITK, KIR, LAG3, LAMP1, leptin, LPFS2, MHC class II, MUC-1, MUC-16, NCR3LG1, NKG2D, NKp46, NTPDase-1, OX40, OX40L, PD-1, PD-L1, PD-L2, PROM1, S152, SIRPalpha, SISP1, SLC, SPG64, ST2, STEAP1, STEAP2, Syk kinase, STEAP1, TROP2, TACI, TDO, TGFBETA, T14, TIGIT, TIM3, TLR, TLR2, TLR4, TLR5, TLR9, TMEF1, TNFa, TNFRSF7, Tp55, TREM1, TSLP, TSLPR, TWEAK, VEGF, VISTA, Vstm3, or WUCAM. For example, the antigen binding polypeptide complex may specifically bind CD28, CD3, cMet, and Trop2. For example, the antigen binding polypeptide complex may specifically bind CD28, CD3, CD19, and CD20. For example, VL2 and VH2 may specifically bind to cMet. For example, VL3 and VH3 may specifically bind to cMet. For example, VL2 and VH2 may specifically bind to Trop2. For example, VL3 and VH3 may specifically bind to Trop2. For example, VL2 and VH2 may specifically bind to CD19. For example, VL3 and VH3 may specifically bind to CD19. For example, VL2 and VH2 may specifically bind to CD20. For example, VL3 and VH3 may specifically bind to CD20.

In some aspects, the invention is directed to an antigen binding polypeptide complex comprising a first polypeptide and a second polypeptide; wherein the first polypeptide has a structure represented by VL1-L1-VL2-L2-VH2-L3-VH1-L4-Fc; or VH1-L5-VH2-L6-VL2-L7-VL1-L8-Fc; wherein the second polypeptide has a structure represented by VL3-L9-VL4-L10-VH4-L11-VH3-L12-Fc; or VH3-L13-VH4-L14-VL4-L15-VL3-L16-Fc; wherein VL1 is a first immunoglobulin light chain variable region that specifically binds to A2AR, APRIL, ATPDase, BAFF, BAFFR, BCMA, BlyS, BTK, BTLA, B7DC, B7H1, B7H2, B7H3, B7H4, B7H5, B7H6, B7H7, B7RP1, B7-4, C3, C5, CCL2, CCL3, CCL4, CCL5, CCL7, CCL8, CCL11, CCL15, CCL17, CCL19, CCL20, CCL21, CCL25, CCR3, CCR4, CD16A, CD19, CD20, CD24, CD27, CD28, CD30, CD38, CD39, CD40, CD40L, CD47, CD52, CD70, CD80, CD86, CD123, CD133, CD137, CD137L, CD160, CD272, CEACAM5, CLEC9, CLEC91, CRTH2, CSF-1, CSF-2, CSF-3, CXCL1, CXCL2, CXCL4, CXCL12, CXCL13, CXCR3, cMet, CTLA4, DLL3, DLL4, DNGR-1, E-cadherin, EGFR, ENTPD1, EpCAM, FCER1, FCER1A, FCER2, FGFR, FLAP, FOLH1, Gi24, GITR, GITRL, GPR5, GP100, GPRC5D, HER2, HER3, ICOSL, ICOS, HHLA2, HMGB1, HVEM, IDO, IFNa, IgE, IGF1R, IL2Rbeta, IL1, ILIA, IL1B, IL1F10, IL2, IL4, IL4Ra, IL5, IL5R, IL6, IL7, IL7Ra, IL8, IL9, IL9R, IL10, rhILlO, IL12, IL13, IL13Ra1, IL13Ra2, IL15, IL17, IL17Rb, IL18, IL22, IL23, IL25, IL7, IL33, IL35, ITGB4, ITK, KIR, LAG3, LAMP1, leptin, LPFS2, MHC class II, MUC-1, MUC-16, NCR3LG1, NKG2D, NKp46, NTPDase-1, OX40, OX40L, PD-1, PD-L1, PD-L2, PROM1, S152, SIRPalpha, SISP1, SLC, SPG64, ST2, STEAP1, STEAP2, Syk kinase, STEAP1, TROP2, TACI, TDO, TGFBETA, T14, TIGIT, TIM3, TLR, TLR2, TLR4, TLR5, TLR9, TMEF1, TNFa, TNFRSF7, Tp55, TREM1, TSLP, TSLPR, TWEAK, VEGF, VISTA, Vstm3, or WUCAM; VL2 is a second immunoglobulin light chain variable region that specifically binds to CD28; VL3 is a third immunoglobulin light chain variable region that specifically binds to A2AR, APRIL, ATPDase, BAFF, BAFFR, BCMA, BlyS, BTK, BTLA, B7DC, B7H1, B7H2, B7H3, B7H4, B7H5, B7H6, B7H7, B7RP1, B7-4, C3, C5, CCL2, CCL3, CCL4, CCL5, CCL7, CCL8, CCL11, CCL15, CCL17, CCL19, CCL20, CCL21, CCL25, CCR3, CCR4, CD16A, CD19, CD20, CD24, CD27, CD28, CD30, CD38, CD39, CD40, CD40L, CD47, CD52, CD70, CD80, CD86, CD123, CD133, CD137, CD137L, CD160, CD272, CEACAM5, CLEC9, CLEC91, CRTH2, CSF-1, CSF-2, CSF-3, CXCL1, CXCL2, CXCL4, CXCL12, CXCL13, CXCR3, cMet, CTLA4, DLL3, DLL4, DNGR-1, E-cadherin, EGFR, ENTPD1, EpCAM, FCER1, FCER1A, FCER2, FGFR, FLAP, FOLH1, Gi24, GITR, GITRL, GPR5, GP100, GPRC5D, HER2, HER3, ICOSL, ICOS, HHLA2, HMGB1, HVEM, IDO, IFNa, IgE, IGF1R, IL2Rbeta, IL1, ILIA, IL1B, IL1F10, IL2, IL4, IL4Ra, IL5, IL5R, IL6, IL7, IL7Ra, IL8, IL9, IL9R, IL10, rhILlO, IL12, IL13, IL13Ra1, IL13Ra2, IL15, IL17, IL17Rb, IL18, IL22, IL23, IL25, IL7, IL33, IL35, ITGB4, ITK, KIR, LAG3, LAMP1, leptin, LPFS2, MHC class II, MUC-1, MUC-16, NCR3LG1, NKG2D, NKp46, NTPDase-1, OX40, OX40L, PD-1, PD-L1, PD-L2, PROM1, 5152, SIRPalpha, SISP1, SLC, SPG64, ST2, STEAP1, STEAP2, Syk kinase, STEAP1, TROP2, TACI, TDO, TGFBETA, T14, TIGIT, TIM3, TLR, TLR2, TLR4, TLR5, TLR9, TMEF1, TNFa, TNFRSF7, Tp55, TREM1, TSLP, TSLPR, TWEAK, VEGF, VISTA, Vstm3, or WUCAM; VL4 is a fourth immunoglobulin light chain variable region that specifically binds to CD3; VH1 is a first immunoglobulin heavy chain variable region that specifically binds to A2AR, APRIL, ATPDase, BAFF, BAFFR, BCMA, BlyS, BTK, BTLA, B7DC, B7H1, B7H2, B7H3, B7H4, B7H5, B7H6, B7H7, B7RP1, B7-4, C3, C5, CCL2, CCL3, CCL4, CCL5, CCL7, CCL8, CCL11, CCL15, CCL17, CCL19, CCL20, CCL21, CCL25, CCR3, CCR4, CD16A, CD19, CD20, CD24, CD27, CD28, CD30, CD38, CD39, CD40, CD40L, CD47, CD52, CD70, CD80, CD86, CD123, CD133, CD137, CD137L, CD160, CD272, CEACAM5, CLEC9, CLEC91, CRTH2, CSF-1, CSF-2, CSF-3, CXCL1, CXCL2, CXCL4, CXCL12, CXCL13, CXCR3, cMet, CTLA4, DLL3, DLL4, DNGR-1, E-cadherin, EGFR, ENTPD1, EpCAM, FCER1, FCER1A, FCER2, FGFR, FLAP, FOLH1, Gi24, GITR, GITRL, GPR5, GP100, GPRC5D, HER2, HER3, ICOSL, ICOS, HHLA2, HMGB1, HVEM, IDO, IFNa, IgE, IGF1R, IL2Rbeta, IL1, ILIA, IL1B, IL1F10, IL2, IL4, IL4Ra, IL5, IL5R, IL6, IL7, IL7Ra, IL8, IL9, IL9R, IL10, rhILlO, IL12, IL13, IL13Ra1, IL13Ra2, IL15, IL17, IL17Rb, IL18, IL22, IL23, IL25, IL7, IL33, IL35, ITGB4, ITK, KIR, LAG3, LAMP1, leptin, LPFS2, MHC class II, MUC-1, MUC-16, NCR3LG1, NKG2D, NKp46, NTPDase-1, OX40, OX40L, PD-1, PD-L1, PD-L2, PROM1, S152, SIRPalpha, SISP1, SLC, SPG64, ST2, STEAP1, STEAP2, Syk kinase, STEAP1, TROP2, TACI, TDO, TGFBETA, T14, TIGIT, TIM3, TLR, TLR2, TLR4, TLR5, TLR9, TMEF1, TNFa, TNFRSF7, Tp55, TREM1, TSLP, TSLPR, TWEAK, VEGF, VISTA, Vstm3, or WUCAM; VH2 is a second immunoglobulin heavy chain variable region that specifically binds to CD28; VH3 is a third immunoglobulin heavy chain variable region that specifically binds to A2AR, APRIL, ATPDase, BAFF, BAFFR, BCMA, BlyS, BTK, BTLA, B7DC, B7H1, B7H2, B7H3, B7H4, B7H5, B7H6, B7H7, B7RP1, B7-4, C3, C5, CCL2, CCL3, CCL4, CCL5, CCL7, CCL8, CCL11, CCL15, CCL17, CCL19, CCL20, CCL21, CCL25, CCR3, CCR4, CD16A, CD19, CD20, CD24, CD27, CD28, CD30, CD38, CD39, CD40, CD40L, CD47, CD52, CD70, CD80, CD86, CD123, CD133, CD137, CD137L, CD160, CD272, CEACAM5, CLEC9, CLEC91, CRTH2, CSF-1, CSF-2, CSF-3, CXCL1, CXCL2, CXCL4, CXCL12, CXCL13, CXCR3, cMet, CTLA4, DLL3, DLL4, DNGR-1, E-cadherin, EGFR, ENTPD1, EpCAM, FCER1, FCER1A, FCER2, FGFR, FLAP, FOLH1, Gi24, GITR, GITRL, GPR5, GP100, GPRC5D, HER2, HER3, ICOSL, ICOS, HHLA2, HMGB1, HVEM, IDO, IFNa, IgE, IGF1R, IL2Rbeta, IL1, ILIA, IL1B, IL1F10, IL2, IL4, IL4Ra, IL5, IL5R, IL6, IL7, IL7Ra, IL8, IL9, IL9R, IL10, rhILlO, IL12, IL13, IL13Ra1, IL13Ra2, IL15, IL17, IL17Rb, IL18, IL22, IL23, IL25, IL7, IL33, IL35, ITGB4, ITK, KIR, LAG3, LAMP1, leptin, LPFS2, MHC class II, MUC-1, MUC-16, NCR3LG1, NKG2D, NKp46, NTPDase-1, OX40, OX40L, PD-1, PD-L1, PD-L2, PROM1, S152, SIRPalpha, SISP1, SLC, SPG64, ST2, STEAP1, STEAP2, Syk kinase, STEAP1, TROP2, TACI, TDO, TGFBETA, T14, TIGIT, TIM3, TLR, TLR2, TLR4, TLR5, TLR9, TMEF1, TNFa, TNFRSF7, Tp55, TREM1, TSLP, TSLPR, TWEAK, VEGF, VISTA, Vstm3, or WUCAM; VH4 is a fourth immunoglobulin heavy chain variable region that specifically binds to CD3; Fc is a region comprising an immunoglobulin heavy chain constant region 2 (CH2), an immunoglobulin heavy chain constant region 3 (CH3), and optionally, an immunoglobulin hinge; and L1-L16 are amino acid linkers. In some aspects, the first polypeptide has a structure represented by VL1-L1-VL2-L2-VH2-L3-VH1-L4-Fc and the second polypeptide has a structure represented by VL3-L9-VL4-L10-VH4-L11-VH3-L12-Fc. In some aspects, the first polypeptide has a structure represented by VL1-L1-VL2-L2-VH2-L3-VH1-L4-Fc and the second polypeptide has a structure represented by VH3-L13-VH4-L14-VL4-L15-VL3-L16-Fc. In some aspects, the first polypeptide has a structure represented by VH1-L5-VH2-L6-VL2-L7-VL1-L8-Fc and the second polypeptide has a structure represented by VL3-L9-VL4-L10-VH4-L11-VH3-L12-Fc. In some aspects, the first polypeptide has a structure represented by VH1-L5-VH2-L6-VL2-L7-VL1-L8-Fc and the second polypeptide has a structure represented by VH3-L13-VH4-L14-VL4-L15-VL3-L16-Fc. In some aspects, VL1 and VH1 may specifically bind to A2AR, APRIL, ATPDase, BAFF, BAFFR, BCMA, BlyS, BTK, BTLA, B7DC, B7H1, B7H2, B7H3, B7H4, B7H5, B7H6, B7H7, B7RP1, B7-4, C3, C5, CCL2, CCL3, CCL4, CCL5, CCL7, CCL8, CCL11, CCL15, CCL17, CCL19, CCL20, CCL21, CCL25, CCR3, CCR4, CD16A, CD19, CD20, CD24, CD27, CD28, CD30, CD38, CD39, CD40, CD40L, CD47, CD52, CD70, CD80, CD86, CD123, CD133, CD137, CD137L, CD160, CD272, CEACAM5, CLEC9, CLEC91, CRTH2, CSF-1, CSF-2, CSF-3, CXCL1, CXCL2, CXCL4, CXCL12, CXCL13, CXCR3, cMet, CTLA4, DLL3, DLL4, DNGR-1, E-cadherin, EGFR, ENTPD1, EpCAM, FCER1, FCER1A, FCER2, FGFR, FLAP, FOLH1, Gi24, GITR, GITRL, GPR5, GP100, GPRC5D, HER2, HER3, ICOSL, ICOS, HHLA2, HMGB1, HVEM, IDO, IFNa, IgE, IGF1R, IL2Rbeta, IL1, ILIA, IL1B, IL1F10, IL2, IL4, IL4Ra, IL5, IL5R, IL6, IL7, IL7Ra, IL8, IL9, IL9R, IL10, rhILlO, IL12, IL13, IL13Ra1, IL13Ra2, IL15, IL17, IL17Rb, IL18, IL22, IL23, IL25, IL7, IL33, IL35, ITGB4, ITK, KIR, LAG3, LAMP1, leptin, LPFS2, MHC class II, MUC-1, MUC-16, NCR3LG1, NKG2D, NKp46, NTPDase-1, OX40, OX40L, PD-1, PD-L1, PD-L2, PROM1, S152, SIRPalpha, SISP1, SLC, SPG64, ST2, STEAP1, STEAP2, Syk kinase, STEAP1, TROP2, TACI, TDO, TGFBETA, T14, TIGIT, TIM3, TLR, TLR2, TLR4, TLR5, TLR9, TMEF1, TNFa, TNFRSF7, Tp55, TREM1, TSLP, TSLPR, TWEAK, VEGF, VISTA, Vstm3, or WUCAM. In some aspects, VL3 and VH3 may specifically bind to A2AR, APRIL, ATPDase, BAFF, BAFFR, BCMA, BlyS, BTK, BTLA, B7DC, B7H1, B7H2, B7H3, B7H4, B7H5, B7H6, B7H7, B7RP1, B7-4, C3, C5, CCL2, CCL3, CCL4, CCL5, CCL7, CCL8, CCL11, CCL15, CCL17, CCL19, CCL20, CCL21, CCL25, CCR3, CCR4, CD16A, CD19, CD20, CD24, CD27, CD28, CD30, CD38, CD39, CD40, CD40L, CD47, CD52, CD70, CD80, CD86, CD123, CD133, CD137, CD137L, CD160, CD272, CEACAM5, CLEC9, CLEC91, CRTH2, CSF-1, CSF-2, CSF-3, CXCL1, CXCL2, CXCL4, CXCL12, CXCL13, CXCR3, cMet, CTLA4, DLL3, DLL4, DNGR-1, E-cadherin, EGFR, ENTPD1, EpCAM, FCER1, FCER1A, FCER2, FGFR, FLAP, FOLH1, Gi24, GITR, GITRL, GPR5, GP100, GPRC5D, HER2, HER3, ICOSL, ICOS, HHLA2, HMGB1, HVEM, IDO, IFNa, IgE, IGF1R, IL2Rbeta, IL1, ILIA, IL1B, IL1F10, IL2, IL4, IL4Ra, IL5, IL5R, IL6, IL7, IL7Ra, IL8, IL9, IL9R, IL10, rhILlO, IL12, IL13, IL13Ra1, IL13Ra2, IL15, IL17, IL17Rb, IL18, IL22, IL23, IL25, IL7, IL33, IL35, ITGB4, ITK, KIR, LAG3, LAMP1, leptin, LPFS2, MHC class II, MUC-1, MUC-16, NCR3LG1, NKG2D, NKp46, NTPDase-1, OX40, OX40L, PD-1, PD-L1, PD-L2, PROM1, S152, SIRPalpha, SISP1, SLC, SPG64, ST2, STEAP1, STEAP2, Syk kinase, STEAP1, TROP2, TACI, TDO, TGFBETA, T14, TIGIT, TIM3, TLR, TLR2, TLR4, TLR5, TLR9, TMEF1, TNFa, TNFRSF7, Tp55, TREM1, TSLP, TSLPR, TWEAK, VEGF, VISTA, Vstm3, or WUCAM. For example, the antigen binding polypeptide complex may specifically bind CD28, CD3, cMet, and Trop2. For example, the antigen binding polypeptide complex may specifically bind CD28, CD3, CD19, and CD20. For example, VL1 and VH1 may specifically bind to cMet. For example, VL3 and VH3 may specifically bind to cMet. For example, VL1 and VH1 may specifically bind to Trop2. For example, VL3 and VH3 may specifically bind to Trop2. For example, VL1 and VH1 may specifically bind to CD19. For example, VL3 and VH3 may specifically bind to CD19. For example, VL1 and VH1 may specifically bind to CD20. For example, VL3 and VH3 may specifically bind to CD20.

89) In some aspects, the invention is directed to an antigen binding polypeptide complex comprising a first polypeptide and a second polypeptide; wherein the first polypeptide has a structure represented by VL1-L1-VL2-L2-VH2-L3-VH1-L4-Fc; or VH1-L5-VH2-L6-VL2-L7-VL1-L8-Fc; wherein the second polypeptide has a structure represented by VL3-L9-VL4-L10-VH4-L11-VH3-L12-Fc; or VH3-L13-VH4-L14-VL4-L15-VL3-L16-Fc; wherein VL1 is a first immunoglobulin light chain variable region that specifically binds to A2AR, APRIL, ATPDase, BAFF, BAFFR, BCMA, BlyS, BTK, BTLA, B7DC, B7H1, B7H2, B7H3, B7H4, B7H5, B7H6, B7H7, B7RP1, B7-4, C3, C5, CCL2, CCL3, CCL4, CCL5, CCL7, CCL8, CCL11, CCL15, CCL17, CCL19, CCL20, CCL21, CCL25, CCR3, CCR4, CD16A, CD19, CD20, CD24, CD27, CD28, CD30, CD38, CD39, CD40, CD40L, CD47, CD52, CD70, CD80, CD86, CD123, CD133, CD137, CD137L, CD160, CD272, CEACAM5, CLEC9, CLEC91, CRTH2, CSF-1, CSF-2, CSF-3, CXCL1, CXCL2, CXCL4, CXCL12, CXCL13, CXCR3, cMet, CTLA4, DLL3, DLL4, DNGR-1, E-cadherin, EGFR, ENTPD1, EpCAM, FCER1, FCER1A, FCER2, FGFR, FLAP, FOLH1, Gi24, GITR, GITRL, GPR5, GP100, GPRC5D, HER2, HER3, ICOSL, ICOS, HHLA2, HMGB1, HVEM, IDO, IFNa, IgE, IGF1R, IL2Rbeta, IL1, ILIA, IL1B, IL1F10, IL2, IL4, IL4Ra, IL5, IL5R, IL6, IL7, IL7Ra, IL8, IL9, IL9R, IL10, rhILlO, IL12, IL13, IL13Ra1, IL13Ra2, IL15, IL17, IL17Rb, IL18, IL22, IL23, IL25, IL7, IL33, IL35, ITGB4, ITK, KIR, LAG3, LAMP1, leptin, LPFS2, MHC class II, MUC-1, MUC-16, NCR3LG1, NKG2D, NKp46, NTPDase-1, OX40, OX40L, PD-1, PD-L1, PD-L2, PROM1, S152, SIRPalpha, SISP1, SLC, SPG64, ST2, STEAP1, STEAP2, Syk kinase, STEAP1, TROP2, TACI, TDO, TGFBETA, T14, TIGIT, TIM3, TLR, TLR2, TLR4, TLR5, TLR9, TMEF1, TNFa, TNFRSF7, Tp55, TREM1, TSLP, TSLPR, TWEAK, VEGF, VISTA, Vstm3, or WUCAM; VL2 is a second immunoglobulin light chain variable region that specifically binds to A2AR, APRIL, ATPDase, BAFF, BAFFR, BCMA, BlyS, BTK, BTLA, B7DC, B7H1, B7H2, B7H3, B7H4, B7H5, B7H6, B7H7, B7RP1, B7-4, C3, C5, CCL2, CCL3, CCL4, CCL5, CCL7, CCL8, CCL11, CCL15, CCL17, CCL19, CCL20, CCL21, CCL25, CCR3, CCR4, CD16A, CD19, CD20, CD24, CD27, CD28, CD30, CD38, CD39, CD40, CD40L, CD47, CD52, CD70, CD80, CD86, CD123, CD133, CD137, CD137L, CD160, CD272, CEACAM5, CLEC9, CLEC91, CRTH2, CSF-1, CSF-2, CSF-3, CXCL1, CXCL2, CXCL4, CXCL12, CXCL13, CXCR3, cMet, CTLA4, DLL3, DLL4, DNGR-1, E-cadherin, EGFR, ENTPD1, EpCAM, FCER1, FCER1A, FCER2, FGFR, FLAP, FOLH1, Gi24, GITR, GITRL, GPR5, GP100, GPRC5D, HER2, HER3, ICOSL, ICOS, HHLA2, HMGB1, HVEM, IDO, IFNa, IgE, IGF1R, IL2Rbeta, IL1, ILIA, IL1B, IL1F10, IL2, IL4, IL4Ra, IL5, IL5R, IL6, IL7, IL7Ra, IL8, IL9, IL9R, IL10, rhILlO, IL12, IL13, IL13Ra1, IL13Ra2, IL15, IL17, IL17Rb, IL18, IL22, IL23, IL25, IL7, IL33, IL35, ITGB4, ITK, KIR, LAG3, LAMP1, leptin, LPFS2, MHC class II, MUC-1, MUC-16, NCR3LG1, NKG2D, NKp46, NTPDase-1, OX40, OX40L, PD-1, PD-L1, PD-L2, PROM1, S152, SIRPalpha, SISP1, SLC, SPG64, ST2, STEAP1, STEAP2, Syk kinase, STEAP1, TROP2, TACI, TDO, TGFBETA, T14, TIGIT, TIM3, TLR, TLR2, TLR4, TLR5, TLR9, TMEF1, TNFa, TNFRSF7, Tp55, TREM1, TSLP, TSLPR, TWEAK, VEGF, VISTA, Vstm3, or WUCAM; VL3 is a third immunoglobulin light chain variable region that specifically binds to CD28; VL4 is a fourth immunoglobulin light chain variable region that specifically binds to CD3; VH1 is a first immunoglobulin heavy chain variable region that specifically binds to A2AR, APRIL, ATPDase, BAFF, BAFFR, BCMA, BlyS, BTK, BTLA, B7DC, B7H1, B7H2, B7H3, B7H4, B7H5, B7H6, B7H7, B7RP1, B7-4, C3, C5, CCL2, CCL3, CCL4, CCL5, CCL7, CCL8, CCL11, CCL15, CCL17, CCL19, CCL20, CCL21, CCL25, CCR3, CCR4, CD16A, CD19, CD20, CD24, CD27, CD28, CD30, CD38, CD39, CD40, CD40L, CD47, CD52, CD70, CD80, CD86, CD123, CD133, CD137, CD137L, CD160, CD272, CEACAM5, CLEC9, CLEC91, CRTH2, CSF-1, CSF-2, CSF-3, CXCL1, CXCL2, CXCL4, CXCL12, CXCL13, CXCR3, cMet, CTLA4, DLL3, DLL4, DNGR-1, E-cadherin, EGFR, ENTPD1, EpCAM, FCER1, FCER1A, FCER2, FGFR, FLAP, FOLH1, Gi24, GITR, GITRL, GPR5, GP100, GPRC5D, HER2, HER3, ICOSL, ICOS, HHLA2, HMGB1, HVEM, IDO, IFNa, IgE, IGF1R, IL2Rbeta, IL1, ILIA, IL1B, IL1F10, IL2, IL4, IL4Ra, IL5, IL5R, IL6, IL7, IL7Ra, IL8, IL9, IL9R, IL10, rhILlO, IL12, IL13, IL13Ra1, IL13Ra2, IL15, IL17, IL17Rb, IL18, IL22, IL23, IL25, IL7, IL33, IL35, ITGB4, ITK, KIR, LAG3, LAMP1, leptin, LPFS2, MHC class II, MUC-1, MUC-16, NCR3LG1, NKG2D, NKp46, NTPDase-1, OX40, OX40L, PD-1, PD-L1, PD-L2, PROM1, 5152, SIRPalpha, SISP1, SLC, SPG64, ST2, STEAP1, STEAP2, Syk kinase, STEAP1, TROP2, TACI, TDO, TGFBETA, T14, TIGIT, TIM3, TLR, TLR2, TLR4, TLR5, TLR9, TMEF1, TNFa, TNFRSF7, Tp55, TREM1, TSLP, TSLPR, TWEAK, VEGF, VISTA, Vstm3, or WUCAM; VH2 is a second immunoglobulin heavy chain variable region that specifically binds to A2AR, APRIL, ATPDase, BAFF, BAFFR, BCMA, BlyS, BTK, BTLA, B7DC, B7H1, B7H2, B7H3, B7H4, B7H5, B7H6, B7H7, B7RP1, B7-4, C3, C5, CCL2, CCL3, CCL4, CCL5, CCL7, CCL8, CCL11, CCL15, CCL17, CCL19, CCL20, CCL21, CCL25, CCR3, CCR4, CD16A, CD19, CD20, CD24, CD27, CD28, CD30, CD38, CD39, CD40, CD40L, CD47, CD52, CD70, CD80, CD86, CD123, CD133, CD137, CD137L, CD160, CD272, CEACAM5, CLEC9, CLEC91, CRTH2, CSF-1, CSF-2, CSF-3, CXCL1, CXCL2, CXCL4, CXCL12, CXCL13, CXCR3, cMet, CTLA4, DLL3, DLL4, DNGR-1, E-cadherin, EGFR, ENTPD1, EpCAM, FCER1, FCER1A, FCER2, FGFR, FLAP, FOLH1, Gi24, GITR, GITRL, GPR5, GP100, GPRC5D, HER2, HER3, ICOSL, ICOS, HHLA2, HMGB1, HVEM, IDO, IFNa, IgE, IGF1R, IL2Rbeta, IL1, ILIA, IL1B, IL1F10, IL2, IL4, IL4Ra, IL5, IL5R, IL6, IL7, IL7Ra, IL8, IL9, IL9R, IL10, rhILlO, IL12, IL13, IL13Ra1, IL13Ra2, IL15, IL17, IL17Rb, IL18, IL22, IL23, IL25, IL7, IL33, IL35, ITGB4, ITK, KIR, LAG3, LAMP1, leptin, LPFS2, MHC class II, MUC-1, MUC-16, NCR3LG1, NKG2D, NKp46, NTPDase-1, OX40, OX40L, PD-1, PD-L1, PD-L2, PROM1, S152, SIRPalpha, SISP1, SLC, SPG64, ST2, STEAP1, STEAP2, Syk kinase, STEAP1, TROP2, TACI, TDO, TGFBETA, T14, TIGIT, TIM3, TLR, TLR2, TLR4, TLR5, TLR9, TMEF1, TNFa, TNFRSF7, Tp55, TREM1, TSLP, TSLPR, TWEAK, VEGF, VISTA, Vstm3, or WUCAM; VH3 is a third immunoglobulin heavy chain variable region that specifically binds to CD28; VH4 is a fourth immunoglobulin heavy chain variable region that specifically binds to CD3; Fc is a region comprising an immunoglobulin heavy chain constant region 2 (CH2), an immunoglobulin heavy chain constant region 3 (CH3), and optionally, an immunoglobulin hinge; and L1-L16 are amino acid linkers. In some aspects, the first polypeptide has a structure represented by VL1-L1-VL2-L2-VH2-L3-VH1-L4-Fc and the second polypeptide has a structure represented by VL3-L9-VL4-L10-VH4-L11-VH3-L12-Fc. In some aspects, the first polypeptide has a structure represented by VL1-L1-VL2-L2-VH2-L3-VH1-L4-Fc and the second polypeptide has a structure represented by VH3-L13-VH4-L14-VL4-L15-VL3-L16-Fc. In some aspects, the first polypeptide has a structure represented by VH1-L5-VH2-L6-VL2-L7-VL1-L8-Fc and the second polypeptide has a structure represented by VL3-L9-VL4-L10-VH4-L11-VH3-L12-Fc. In some aspects, the first polypeptide has a structure represented by VH1-L5-VH2-L6-VL2-L7-VL1-L8-Fc and the second polypeptide has a structure represented by VH3-L13-VH4-L14-VL4-L15-VL3-L16-Fc. In some aspects, VL1 and VH1 may specifically bind to A2AR, APRIL, ATPDase, BAFF, BAFFR, BCMA, BlyS, BTK, BTLA, B7DC, B7H1, B7H2, B7H3, B7H4, B7H5, B7H6, B7H7, B7RP1, B7-4, C3, C5, CCL2, CCL3, CCL4, CCL5, CCL7, CCL8, CCL11, CCL15, CCL17, CCL19, CCL20, CCL21, CCL25, CCR3, CCR4, CD16A, CD19, CD20, CD24, CD27, CD28, CD30, CD38, CD39, CD40, CD40L, CD47, CD52, CD70, CD80, CD86, CD123, CD133, CD137, CD137L, CD160, CD272, CEACAM5, CLEC9, CLEC91, CRTH2, CSF-1, CSF-2, CSF-3, CXCL1, CXCL2, CXCL4, CXCL12, CXCL13, CXCR3, cMet, CTLA4, DLL3, DLL4, DNGR-1, E-cadherin, EGFR, ENTPD1, EpCAM, FCER1, FCER1A, FCER2, FGFR, FLAP, FOLH1, Gi24, GITR, GITRL, GPR5, GP100, GPRC5D, HER2, HER3, ICOSL, ICOS, HHLA2, HMGB1, HVEM, IDO, IFNa, IgE, IGF1R, IL2Rbeta, IL1, ILIA, IL1B, IL1F10, IL2, IL4, IL4Ra, IL5, IL5R, IL6, IL7, IL7Ra, IL8, IL9, IL9R, IL10, rhILlO, IL12, IL13, IL13Ra1, IL13Ra2, IL15, IL17, IL17Rb, IL18, IL22, IL23, IL25, IL7, IL33, IL35, ITGB4, ITK, KIR, LAG3, LAMP1, leptin, LPFS2, MHC class II, MUC-1, MUC-16, NCR3LG1, NKG2D, NKp46, NTPDase-1, OX40, OX40L, PD-1, PD-L1, PD-L2, PROM1, S152, SIRPalpha, SISP1, SLC, SPG64, ST2, STEAP1, STEAP2, Syk kinase, STEAP1, TROP2, TACI, TDO, TGFBETA, T14, TIGIT, TIM3, TLR, TLR2, TLR4, TLR5, TLR9, TMEF1, TNFa, TNFRSF7, Tp55, TREM1, TSLP, TSLPR, TWEAK, VEGF, VISTA, Vstm3, or WUCAM. In some aspects, VL2 and VH2 may specifically bind to A2AR, APRIL, ATPDase, BAFF, BAFFR, BCMA, BlyS, BTK, BTLA, B7DC, B7H1, B7H2, B7H3, B7H4, B7H5, B7H6, B7H7, B7RP1, B7-4, C3, C5, CCL2, CCL3, CCL4, CCL5, CCL7, CCL8, CCL11, CCL15, CCL17, CCL19, CCL20, CCL21, CCL25, CCR3, CCR4, CD16A, CD19, CD20, CD24, CD27, CD28, CD30, CD38, CD39, CD40, CD40L, CD47, CD52, CD70, CD80, CD86, CD123, CD133, CD137, CD137L, CD160, CD272, CEACAM5, CLEC9, CLEC91, CRTH2, CSF-1, CSF-2, CSF-3, CXCL1, CXCL2, CXCL4, CXCL12, CXCL13, CXCR3, cMet, CTLA4, DLL3, DLL4, DNGR-1, E-cadherin, EGFR, ENTPD1, EpCAM, FCER1, FCER1A, FCER2, FGFR, FLAP, FOLH1, Gi24, GITR, GITRL, GPR5, GP100, GPRC5D, HER2, HER3, ICOSL, ICOS, HHLA2, HMGB1, HVEM, IDO, IFNa, IgE, IGF1R, IL2Rbeta, IL1, ILIA, IL1B, IL1F10, IL2, IL4, IL4Ra, IL5, IL5R, IL6, IL7, IL7Ra, IL8, IL9, IL9R, IL10, rhILlO, IL12, IL13, IL13Ra1, IL13Ra2, IL15, IL17, IL17Rb, IL18, IL22, IL23, IL25, IL7, IL33, IL35, ITGB4, ITK, KIR, LAG3, LAMP1, leptin, LPFS2, MHC class II, MUC-1, MUC-16, NCR3LG1, NKG2D, NKp46, NTPDase-1, OX40, OX40L, PD-1, PD-L1, PD-L2, PROM1, S152, SIRPalpha, SISP1, SLC, SPG64, ST2, STEAP1, STEAP2, Syk kinase, STEAP1, TROP2, TACI, TDO, TGFBETA, T14, TIGIT, TIM3, TLR, TLR2, TLR4, TLR5, TLR9, TMEF1, TNFa, TNFRSF7, Tp55, TREM1, TSLP, TSLPR, TWEAK, VEGF, VISTA, Vstm3, or WUCAM. For example, the antigen binding polypeptide complex may specifically bind CD28, CD3, cMet, and Trop2. For example, the antigen binding polypeptide complex may specifically bind CD28, CD3, CD19, and CD20. For example, VL1 and VH1 may specifically bind to cMet. For example, VL2 and VH2 may specifically bind to cMet. For example, VL1 and VH1 may specifically bind to Trop2. For example, VL2 and VH2 may specifically bind to Trop2. For example, VL1 and VH1 may specifically bind to CD19. For example, VL2 and VH2 may specifically bind to CD19. For example, VL1 and VH1 may specifically bind to CD20. For example, VL2 and VH2 may specifically bind to CD20.

In some aspects of the antigen binding polypeptide complex described herein, VL4 and VH4 specifically bind to CD3, VL3 and VH3 specifically bind to CD28, VL2 and VH2 specifically bind to Trop2, and VL1 and VH1 specifically bind to cMet. In some aspects, VL4 and VH4 specifically bind to CD3, VL3 and VH3 specifically bind to CD28, VL2 and VH2 specifically bind to cMet, and VL1 and VH1 specifically bind to Trop2. In some aspects, VL4 and VH4 specifically bind to CD3, VL3 and VH3 specifically bind to Trop2, VL2 and VH2 specifically bind to CD28, and VL1 and VH1 specifically bind to cMet. In some aspects, VL4 and VH4 specifically bind to CD3, VL3 and VH3 specifically bind to Trop2, VL2 and VH2 specifically bind to cMet, and VL1 and VH1 specifically bind to CD28. In some aspects, VL4 and VH4 specifically bind to CD3, VL3 and VH3 specifically bind to cMet, VL2 and VH2 specifically bind to Trop2, and VL1 and VH1 specifically bind to CD28. In some aspects, VL4 and VH4 specifically bind to CD3, VL3 and VH3 specifically bind to cMet, VL2 and VH2 specifically bind to Trop2, and VL1 and VH1 specifically bind to CD28. In some aspects, VL4 and VH4 specifically bind to CD3, VL3 and VH3 specifically bind to CD28, VL2 and VH2 specifically bind to CD20, and VL1 and VH1 specifically bind to CD19. In some aspects, VL4 and VH4 specifically bind to CD3, VL3 and VH3 specifically bind to CD28, VL2 and VH2 specifically bind to CD19, and VL1 and VH1 specifically bind to CD20. In some aspects, VL4 and VH4 specifically bind to CD3, VL3 and VH3 specifically bind to CD20, VL2 and VH2 specifically bind to CD28, and VL1 and VH1 specifically bind to CD19. In some aspects, VL4 and VH4 specifically bind to CD3, VL3 and VH3 specifically bind to CD20, VL2 and VH2 specifically bind to CD19, and VL1 and VH1 specifically bind to CD28. In some aspects, VL4 and VH4 specifically bind to CD3, VL3 and VH3 specifically bind to CD19, VL2 and VH2 specifically bind to CD20, and VL1 and VH1 specifically bind to CD28. In some aspects, VL4 and VH4 specifically bind to CD3, VL3 and VH3 specifically bind to CD19, VL2 and VH2 specifically bind to CD28, and VL1 and VH1 specifically bind to CD20.

In some aspects, the invention is directed to an antigen binding polypeptide complex comprising a first polypeptide and a second polypeptide; wherein the first polypeptide has a structure represented by VL1-L1-VL2-L2-VH2-L3-VH1-L4-CH1-L5-Fc; VH1-L6-VH2-L7-VL2-L8-VL1-L9-CH1-L10-Fc; VL1-L11-VL2-L12-VH2-L13-VH1-L14-CL-L15-Fc; VH1-L16-VH2-L17-VL2-L18-VL1-L19-CL-L20-Fc; VL1-L21-VL2-L22-VH2-L23-VH1-L24-CH1-L25-CL-L26-Fc; VH1-L27-VH2-L28-VL2-L29-VL1-L30-CH1-L31-CL-L32-Fc; VL1-L33-VL2-L34-VH2-L35-VH1-L36-CL-L37-CH1-L38-Fc; or VH1-L39-VH2-L40-VL2-L41-VL1-L42-CL-L43-CH1-L44-Fc; wherein the second polypeptide has a structure represented by VL3-L45-VL4-L46-VH4-L47-VH3-L48-CH1-L49-Fc; VH3-L50-VH4-L51-VL4-L52-VL3-L53-CH1-L54-Fc; VL3-L55-VL4-L56-VH4-L57-VH3-L58-CL-L59-Fc; VH3-L60-VH4-L61-VL4-L62-VL3-L63-CL-L64-Fc; VL3-L65-VL4-L66-VH4-L67-VH3-L68-CH1-L69-CL-L70-Fc; VH3-L71-VH4-L72-VL4-L73-VL3-L74-CH1-L75-CL-L76-Fc; VL3-L77-VL4-L78-VH4-L79-VH3-L80-CL-L81-CH1-L82-Fc; or VH3-L83-VH4-L84-VL4-L85-VL3-L86-CL-L87-CH1-L88-Fc; wherein VL1 is a first immunoglobulin light chain variable region that specifically binds to A2AR, APRIL, ATPDase, BAFF, BAFFR, BCMA, BlyS, BTK, BTLA, B7DC, B7H1, B7H2, B7H3, B7H4, B7H5, B7H6, B7H7, B7RP1, B7-4, C3, C5, CCL2, CCL3, CCL4, CCL5, CCL7, CCL8, CCL11, CCL15, CCL17, CCL19, CCL20, CCL21, CCL25, CCR3, CCR4, CD16A, CD19, CD20, CD24, CD27, CD28, CD30, CD38, CD39, CD40, CD40L, CD47, CD52, CD70, CD80, CD86, CD123, CD133, CD137, CD137L, CD160, CD272, CEACAM5, CLEC9, CLEC91, CRTH2, CSF-1, CSF-2, CSF-3, CXCL1, CXCL2, CXCL4, CXCL12, CXCL13, CXCR3, cMet, CTLA4, DLL3, DLL4, DNGR-1, E-cadherin, EGFR, ENTPD1, EpCAM, FCER1, FCER1A, FCER2, FGFR, FLAP, FOLH1, Gi24, GITR, GITRL, GPR5, GP100, GPRC5D, HER2, HER3, ICOSL, ICOS, HHLA2, HMGB1, HVEM, IDO, IFNa, IgE, IGF1R, IL2Rbeta, IL1, ILIA, IL1B, IL1F10, IL2, IL4, IL4Ra, IL5, IL5R, IL6, IL7, IL7Ra, IL8, IL9, IL9R, IL10, rhILlO, IL12, IL13, IL13Ra1, IL13Ra2, IL15, IL17, IL17Rb, IL18, IL22, IL23, IL25, IL7, IL33, IL35, ITGB4, ITK, KIR, LAG3, LAMP1, leptin, LPFS2, MHC class II, MUC-1, MUC-16, NCR3LG1, NKG2D, NKp46, NTPDase-1, OX40, OX40L, PD-1, PD-L1, PD-L2, PROM1, S152, SIRPalpha, SISP1, SLC, SPG64, ST2, STEAP1, STEAP2, Syk kinase, STEAP1, TROP2, TACI, TDO, TGFBETA, T14, TIGIT, TIM3, TLR, TLR2, TLR4, TLR5, TLR9, TMEF1, TNFa, TNFRSF7, Tp55, TREM1, TSLP, TSLPR, TWEAK, VEGF, VISTA, Vstm3, or WUCAM; VL2 is a second immunoglobulin light chain variable region that specifically binds to A2AR, APRIL, ATPDase, BAFF, BAFFR, BCMA, BlyS, BTK, BTLA, B7DC, B7H1, B7H2, B7H3, B7H4, B7H5, B7H6, B7H7, B7RP1, B7-4, C3, C5, CCL2, CCL3, CCL4, CCL5, CCL7, CCL8, CCL11, CCL15, CCL17, CCL19, CCL20, CCL21, CCL25, CCR3, CCR4, CD16A, CD19, CD20, CD24, CD27, CD28, CD30, CD38, CD39, CD40, CD40L, CD47, CD52, CD70, CD80, CD86, CD123, CD133, CD137, CD137L, CD160, CD272, CEACAM5, CLEC9, CLEC91, CRTH2, CSF-1, CSF-2, CSF-3, CXCL1, CXCL2, CXCL4, CXCL12, CXCL13, CXCR3, cMet, CTLA4, DLL3, DLL4, DNGR-1, E-cadherin, EGFR, ENTPD1, EpCAM, FCER1, FCER1A, FCER2, FGFR, FLAP, FOLH1, Gi24, GITR, GITRL, GPR5, GP100, GPRC5D, HER2, HER3, ICOSL, ICOS, HHLA2, HMGB1, HVEM, IDO, IFNa, IgE, IGF1R, IL2Rbeta, IL1, ILIA, IL1B, IL1F10, IL2, IL4, IL4Ra, IL5, IL5R, IL6, IL7, IL7Ra, IL8, IL9, IL9R, IL10, rhILlO, IL12, IL13, IL13Ra1, IL13Ra2, IL15, IL17, IL17Rb, IL18, IL22, IL23, IL25, IL7, IL33, IL35, ITGB4, ITK, KIR, LAG3, LAMP1, leptin, LPFS2, MHC class II, MUC-1, MUC-16, NCR3LG1, NKG2D, NKp46, NTPDase-1, OX40, OX40L, PD-1, PD-L1, PD-L2, PROM1, S152, SIRPalpha, SISP1, SLC, SPG64, ST2, STEAP1, STEAP2, Syk kinase, STEAP1, TROP2, TACI, TDO, TGFBETA, T14, TIGIT, TIM3, TLR, TLR2, TLR4, TLR5, TLR9, TMEF1, TNFa, TNFRSF7, Tp55, TREM1, TSLP, TSLPR, TWEAK, VEGF, VISTA, Vstm3, or WUCAM; VL3 is a third immunoglobulin light chain variable region that specifically binds to CD3; VL4 is a fourth immunoglobulin light chain variable region that specifically binds to A2AR, APRIL, ATPDase, BAFF, BAFFR, BCMA, BlyS, BTK, BTLA, B7DC, B7H1, B7H2, B7H3, B7H4, B7H5, B7H6, B7H7, B7RP1, B7-4, C3, C5, CCL2, CCL3, CCL4, CCL5, CCL7, CCL8, CCL11, CCL15, CCL17, CCL19, CCL20, CCL21, CCL25, CCR3, CCR4, CD16A, CD19, CD20, CD24, CD27, CD28, CD30, CD38, CD39, CD40, CD40L, CD47, CD52, CD70, CD80, CD86, CD123, CD133, CD137, CD137L, CD160, CD272, CEACAM5, CLEC9, CLEC91, CRTH2, CSF-1, CSF-2, CSF-3, CXCL1, CXCL2, CXCL4, CXCL12, CXCL13, CXCR3, cMet, CTLA4, DLL3, DLL4, DNGR-1, E-cadherin, EGFR, ENTPD1, EpCAM, FCER1, FCER1A, FCER2, FGFR, FLAP, FOLH1, Gi24, GITR, GITRL, GPR5, GP100, GPRC5D, HER2, HER3, ICOSL, ICOS, HHLA2, HMGB1, HVEM, IDO, IFNa, IgE, IGF1R, IL2Rbeta, IL1, ILIA, IL1B, IL1F10, IL2, IL4, IL4Ra, IL5, IL5R, IL6, IL7, IL7Ra, IL8, IL9, IL9R, IL10, rhILlO, IL12, IL13, IL13Ra1, IL13Ra2, IL15, IL17, IL17Rb, IL18, IL22, IL23, IL25, IL7, IL33, IL35, ITGB4, ITK, KIR, LAG3, LAMP1, leptin, LPFS2, MHC class II, MUC-1, MUC-16, NCR3LG1, NKG2D, NKp46, NTPDase-1, OX40, OX40L, PD-1, PD-L1, PD-L2, PROM1, S152, SIRPalpha, SISP1, SLC, SPG64, ST2, STEAP1, STEAP2, Syk kinase, STEAP1, TROP2, TACI, TDO, TGFBETA, T14, TIGIT, TIM3, TLR, TLR2, TLR4, TLR5, TLR9, TMEF1, TNFa, TNFRSF7, Tp55, TREM1, TSLP, TSLPR, TWEAK, VEGF, VISTA, Vstm3, or WUCAM; VH1 is a first immunoglobulin heavy chain variable region that specifically binds to A2AR, APRIL, ATPDase, BAFF, BAFFR, BCMA, BlyS, BTK, BTLA, B7DC, B7H1, B7H2, B7H3, B7H4, B7H5, B7H6, B7H7, B7RP1, B7-4, C3, C5, CCL2, CCL3, CCL4, CCL5, CCL7, CCL8, CCL11, CCL15, CCL17, CCL19, CCL20, CCL21, CCL25, CCR3, CCR4, CD16A, CD19, CD20, CD24, CD27, CD28, CD30, CD38, CD39, CD40, CD40L, CD47, CD52, CD70, CD80, CD86, CD123, CD133, CD137, CD137L, CD160, CD272, CEACAM5, CLEC9, CLEC91, CRTH2, CSF-1, CSF-2, CSF-3, CXCL1, CXCL2, CXCL4, CXCL12, CXCL13, CXCR3, cMet, CTLA4, DLL3, DLL4, DNGR-1, E-cadherin, EGFR, ENTPD1, EpCAM, FCER1, FCER1A, FCER2, FGFR, FLAP, FOLH1, Gi24, GITR, GITRL, GPR5, GP100, GPRC5D, HER2, HER3, ICOSL, ICOS, HHLA2, HMGB1, HVEM, IDO, IFNa, IgE, IGF1R, IL2Rbeta, IL1, ILIA, IL1B, IL1F10, IL2, IL4, IL4Ra, IL5, IL5R, IL6, IL7, IL7Ra, IL8, IL9, IL9R, IL10, rhILlO, IL12, IL13, IL13Ra1, IL13Ra2, IL15, IL17, IL17Rb, IL18, IL22, IL23, IL25, IL7, IL33, IL35, ITGB4, ITK, KIR, LAG3, LAMP1, leptin, LPFS2, MHC class II, MUC-1, MUC-16, NCR3LG1, NKG2D, NKp46, NTPDase-1, OX40, OX40L, PD-1, PD-L1, PD-L2, PROM1, S152, SIRPalpha, SISP1, SLC, SPG64, ST2, STEAP1, STEAP2, Syk kinase, STEAP1, TROP2, TACI, TDO, TGFBETA, T14, TIGIT, TIM3, TLR, TLR2, TLR4, TLR5, TLR9, TMEF1, TNFa, TNFRSF7, Tp55, TREM1, TSLP, TSLPR, TWEAK, VEGF, VISTA, Vstm3, or WUCAM; VH2 is a second immunoglobulin heavy chain variable region that specifically binds to A2AR, APRIL, ATPDase, BAFF, BAFFR, BCMA, BlyS, BTK, BTLA, B7DC, B7H1, B7H2, B7H3, B7H4, B7H5, B7H6, B7H7, B7RP1, B7-4, C3, C5, CCL2, CCL3, CCL4, CCL5, CCL7, CCL8, CCL11, CCL15, CCL17, CCL19, CCL20, CCL21, CCL25, CCR3, CCR4, CD16A, CD19, CD20, CD24, CD27, CD28, CD30, CD38, CD39, CD40, CD40L, CD47, CD52, CD70, CD80, CD86, CD123, CD133, CD137, CD137L, CD160, CD272, CEACAM5, CLEC9, CLEC91, CRTH2, CSF-1, CSF-2, CSF-3, CXCL1, CXCL2, CXCL4, CXCL12, CXCL13, CXCR3, cMet, CTLA4, DLL3, DLL4, DNGR-1, E-cadherin, EGFR, ENTPD1, EpCAM, FCER1, FCER1A, FCER2, FGFR, FLAP, FOLH1, Gi24, GITR, GITRL, GPR5, GP100, GPRC5D, HER2, HER3, ICOSL, ICOS, HHLA2, HMGB1, HVEM, IDO, IFNa, IgE, IGF1R, IL2Rbeta, IL1, ILIA, IL1B, IL1F10, IL2, IL4, IL4Ra, IL5, IL5R, IL6, IL7, IL7Ra, IL8, IL9, IL9R, IL10, rhILlO, IL12, IL13, IL13Ra1, IL13Ra2, IL15, IL17, IL17Rb, IL18, IL22, IL23, IL25, IL7, IL33, IL35, ITGB4, ITK, KIR, LAG3, LAMP1, leptin, LPFS2, MHC class II, MUC-1, MUC-16, NCR3LG1, NKG2D, NKp46, NTPDase-1, OX40, OX40L, PD-1, PD-L1, PD-L2, PROM1, S152, SIRPalpha, SISP1, SLC, SPG64, ST2, STEAP1, STEAP2, Syk kinase, STEAP1, TROP2, TACI, TDO, TGFBETA, T14, TIGIT, TIM3, TLR, TLR2, TLR4, TLR5, TLR9, TMEF1, TNFa, TNFRSF7, Tp55, TREM1, TSLP, TSLPR, TWEAK, VEGF, VISTA, Vstm3, or WUCAM; VH3 is a third immunoglobulin heavy chain variable region that specifically binds to CD3; VH4 is a fourth immunoglobulin heavy chain variable region that specifically binds to A2AR, APRIL, ATPDase, BAFF, BAFFR, BCMA, BlyS, BTK, BTLA, B7DC, B7H1, B7H2, B7H3, B7H4, B7H5, B7H6, B7H7, B7RP1, B7-4, C3, C5, CCL2, CCL3, CCL4, CCL5, CCL7, CCL8, CCL11, CCL15, CCL17, CCL19, CCL20, CCL21, CCL25, CCR3, CCR4, CD16A, CD19, CD20, CD24, CD27, CD28, CD30, CD38, CD39, CD40, CD40L, CD47, CD52, CD70, CD80, CD86, CD123, CD133, CD137, CD137L, CD160, CD272, CEACAM5, CLEC9, CLEC91, CRTH2, CSF-1, CSF-2, CSF-3, CXCL1, CXCL2, CXCL4, CXCL12, CXCL13, CXCR3, cMet, CTLA4, DLL3, DLL4, DNGR-1, E-cadherin, EGFR, ENTPD1, EpCAM, FCER1, FCER1A, FCER2, FGFR, FLAP, FOLH1, Gi24, GITR, GITRL, GPR5, GP100, GPRC5D, HER2, HER3, ICOSL, ICOS, HHLA2, HMGB1, HVEM, IDO, IFNa, IgE, IGF1R, IL2Rbeta, IL1, ILIA, IL1B, IL1F10, IL2, IL4, IL4Ra, IL5, IL5R, IL6, IL7, IL7Ra, IL8, IL9, IL9R, IL10, rhILlO, IL12, IL13, IL13Ra1, IL13Ra2, IL15, IL17, IL17Rb, IL18, IL22, IL23, IL25, IL7, IL33, IL35, ITGB4, ITK, KIR, LAG3, LAMP1, leptin, LPFS2, MHC class II, MUC-1, MUC-16, NCR3LG1, NKG2D, NKp46, NTPDase-1, OX40, OX40L, PD-1, PD-L1, PD-L2, PROM1, S152, SIRPalpha, SISP1, SLC, SPG64, ST2, STEAP1, STEAP2, Syk kinase, STEAP1, TROP2, TACI, TDO, TGFBETA, T14, TIGIT, TIM3, TLR, TLR2, TLR4, TLR5, TLR9, TMEF1, TNFa, TNFRSF7, Tp55, TREM1, TSLP, TSLPR, TWEAK, VEGF, VISTA, Vstm3, or WUCAM; Fc is a region comprising an immunoglobulin heavy chain constant region 2 (CH2), an immunoglobulin heavy chain constant region 3 (CH3), and optionally, an immunoglobulin hinge; CH1 is an immunoglobulin heavy chain constant region 1; CL is an immunoglobulin light chain constant region; and L1-L88 are amino acid linkers. In some aspects, the first polypeptide has a structure represented by VL1-L1-VL2-L2-VH2-L3-VH1-L4-CH1-L5-Fc; VH1-L6-VH2-L7-VL2-L8-VL1-L9-CH1-L10-Fc; VL1-L11-VL2-L12-VH2-L13-VH1-L14-CL-L15-Fc; VH1-L16-VH2-L17-VL2-L18-VL1-L19-CL-L20-Fc; VL1-L21-VL2-L22-VH2-L23-VH1-L24-CH1-L25-CL-L26-Fc; VH1-L27-VH2-L28-VL2-L29-VL1-L30-CH1-L31-CL-L32-Fc; VL1-L33-VL2-L34-VH2-L35-VH1-L36-CL-L37-CH1-L38-Fc; or VH1-L39-VH2-L40-VL2-L41-VL1-L42-CL-L43-CH1-L44-Fc and the second polypeptide has a structure represented by VL3-L45-VL4-L46-VH4-L47-VH3-L48-CH1-L49-Fc. In some aspects, the first polypeptide has a structure represented by VL1-L1-VL2-L2-VH2-L3-VH1-L4-CH1-L5-Fc; VH1-L6-VH2-L7-VL2-L8-VL1-L9-CH1-L10-Fc; VL1-L11-VL2-L12-VH2-L13-VH1-L14-CL-L15-Fc; VH1-L16-VH2-L17-VL2-L18-VL1-L19-CL-L20-Fc; VL1-L21-VL2-L22-VH2-L23-VH1-L24-CH1-L25-CL-L26-Fc; VH1-L27-VH2-L28-VL2-L29-VL1-L30-CH1-L31-CL-L32-Fc; VL1-L33-VL2-L34-VH2-L35-VH1-L36-CL-L37-CH1-L38-Fc; or VH1-L39-VH2-L40-VL2-L41-VL1-L42-CL-L43-CH1-L44-Fc and the second polypeptide has a structure represented by VH3-L50-VH4-L51-VL4-L52-VL3-L53-CH1-L54-Fc. In some aspects, the first polypeptide has a structure represented by VL1-L1-VL2-L2-VH2-L3-VH1-L4-CH1-L5-Fc; VH1-L6-VH2-L7-VL2-L8-VL1-L9-CH1-L10-Fc; VL1-L11-VL2-L12-VH2-L13-VH1-L14-CL-L15-Fc; VH1-L16-VH2-L17-VL2-L18-VL1-L19-CL-L20-Fc; VL1-L21-VL2-L22-VH2-L23-VH1-L24-CH1-L25-CL-L26-Fc; VH1-L27-VH2-L28-VL2-L29-VL1-L30-CH1-L31-CL-L32-Fc; VL1-L33-VL2-L34-VH2-L35-VH1-L36-CL-L37-CH1-L38-Fc; or VH1-L39-VH2-L40-VL2-L41-VL1-L42-CL-L43-CH1-L44-Fc and the second polypeptide has a structure represented by VL3-L55-VL4-L56-VH4-L57-VH3-L58-CL-L59-Fc. In some aspects, the first polypeptide has a structure represented by VL1-L1-VL2-L2-VH2-L3-VH1-L4-CH1-L5-Fc; VH1-L6-VH2-L7-VL2-L8-VL1-L9-CH1-L10-Fc; VL1-L11-VL2-L12-VH2-L13-VH1-L14-CL-L15-Fc; VH1-L16-VH2-L17-VL2-L18-VL1-L19-CL-L20-Fc; VL1-L21-VL2-L22-VH2-L23-VH1-L24-CH1-L25-CL-L26-Fc; VH1-L27-VH2-L28-VL2-L29-VL1-L30-CH1-L31-CL-L32-Fc; VL1-L33-VL2-L34-VH2-L35-VH1-L36-CL-L37-CH1-L38-Fc; or VH1-L39-VH2-L40-VL2-L41-VL1-L42-CL-L43-CH1-L44-Fc and the second polypeptide has a structure represented by VH3-L60-VH4-L61-VL4-L62-VL3-L63-CL-L64-Fc. In some aspects, the first polypeptide has a structure represented by VL1-L1-VL2-L2-VH2-L3-VH1-L4-CH1-L5-Fc; VH1-L6-VH2-L7-VL2-L8-VL1-L9-CH1-L10-Fc; VL1-L11-VL2-L12-VH2-L13-VH1-L14-CL-L15-Fc; VH1-L16-VH2-L17-VL2-L18-VL1-L19-CL-L20-Fc; VL1-L21-VL2-L22-VH2-L23-VH1-L24-CH1-L25-CL-L26-Fc; VH1-L27-VH2-L28-VL2-L29-VL1-L30-CH1-L31-CL-L32-Fc; VL1-L33-VL2-L34-VH2-L35-VH1-L36-CL-L37-CH1-L38-Fc; or VH1-L39-VH2-L40-VL2-L41-VL1-L42-CL-L43-CH1-L44-Fc and the second polypeptide has a structure represented by VL3-L65-VL4-L66-VH4-L67-VH3-L68-CH1-L69-CL-L70-Fc. In some aspects, the first polypeptide has a structure represented by VL1-L1-VL2-L2-VH2-L3-VH1-L4-CH1-L5-Fc; VH1-L6-VH2-L7-VL2-L8-VL1-L9-CH1-L10-Fc; VL1-L11-VL2-L12-VH2-L13-VH1-L14-CL-L15-Fc; VH1-L16-VH2-L17-VL2-L18-VL1-L19-CL-L20-Fc; VL1-L21-VL2-L22-VH2-L23-VH1-L24-CH1-L25-CL-L26-Fc; VH1-L27-VH2-L28-VL2-L29-VL1-L30-CH1-L31-CL-L32-Fc; VL1-L33-VL2-L34-VH2-L35-VH1-L36-CL-L37-CH1-L38-Fc; or VH1-L39-VH2-L40-VL2-L41-VL1-L42-CL-L43-CH1-L44-Fc and the second polypeptide has a structure represented by VH3-L71-VH4-L72-VL4-L73-VL3-L74-CH1-L75-CL-L76-Fc. In some aspects, the first polypeptide has a structure represented by VL1-L1-VL2-L2-VH2-L3-VH1-L4-CH1-L5-Fc; VH1-L6-VH2-L7-VL2-L8-VL1-L9-CH1-L10-Fc; VL1-L11-VL2-L12-VH2-L13-VH1-L14-CL-L15-Fc; VH1-L16-VH2-L17-VL2-L18-VL1-L19-CL-L20-Fc; VL1-L21-VL2-L22-VH2-L23-VH1-L24-CH1-L25-CL-L26-Fc; VH1-L27-VH2-L28-VL2-L29-VL1-L30-CH1-L31-CL-L32-Fc; VL1-L33-VL2-L34-VH2-L35-VH1-L36-CL-L37-CH1-L38-Fc; or VH1-L39-VH2-L40-VL2-L41-VL1-L42-CL-L43-CH1-L44-Fc and the second polypeptide has a structure represented by VL3-L77-VL4-L78-VH4-L79-VH3-L80-CL-L81-CH1-L82-Fc. In some aspects, the first polypeptide has a structure represented by VL1-L1-VL2-L2-VH2-L3-VH1-L4-CH1-L5-Fc; VH1-L6-VH2-L7-VL2-L8-VL1-L9-CH1-L10-Fc; VL1-L11-VL2-L12-VH2-L13-VH1-L14-CL-L15-Fc; VH1-L16-VH2-L17-VL2-L18-VL1-L19-CL-L20-Fc; VL1-L21-VL2-L22-VH2-L23-VH1-L24-CH1-L25-CL-L26-Fc; VH1-L27-VH2-L28-VL2-L29-VL1-L30-CH1-L31-CL-L32-Fc; VL1-L33-VL2-L34-VH2-L35-VH1-L36-CL-L37-CH1-L38-Fc; or VH1-L39-VH2-L40-VL2-L41-VL1-L42-CL-L43-CH1-L44-Fc and the second polypeptide has a structure represented by VH3-L83-VH4-L84-VL4-L85-VL3-L86-CL-L87-CH1-L88-Fc. In some aspects, VL1 and VH1 specifically bind to A2AR, APRIL, ATPDase, BAFF, BAFFR, BCMA, BlyS, BTK, BTLA, B7DC, B7H1, B7H2, B7H3, B7H4, B7H5, B7H6, B7H7, B7RP1, B7-4, C3, C5, CCL2, CCL3, CCL4, CCL5, CCL7, CCL8, CCL11, CCL15, CCL17, CCL19, CCL20, CCL21, CCL25, CCR3, CCR4, CD16A, CD19, CD20, CD24, CD27, CD28, CD30, CD38, CD39, CD40, CD40L, CD47, CD52, CD70, CD80, CD86, CD123, CD133, CD137, CD137L, CD160, CD272, CEACAM5, CLEC9, CLEC91, CRTH2, CSF-1, CSF-2, CSF-3, CXCL1, CXCL2, CXCL4, CXCL12, CXCL13, CXCR3, cMet, CTLA4, DLL3, DLL4, DNGR-1, E-cadherin, EGFR, ENTPD1, EpCAM, FCER1, FCER1A, FCER2, FGFR, FLAP, FOLH1, Gi24, GITR, GITRL, GPR5, GP100, GPRC5D, HER2, HER3, ICOSL, ICOS, HHLA2, HMGB1, HVEM, IDO, IFNa, IgE, IGF1R, IL2Rbeta, IL1, ILIA, IL1B, IL1F10, IL2, IL4, IL4Ra, IL5, IL5R, IL6, IL7, IL7Ra, IL8, IL9, IL9R, IL10, rhILlO, IL12, IL13, IL13Ra1, IL13Ra2, IL15, IL17, IL17Rb, IL18, IL22, IL23, IL25, IL7, IL33, IL35, ITGB4, ITK, KIR, LAG3, LAMP1, leptin, LPFS2, MHC class II, MUC-1, MUC-16, NCR3LG1, NKG2D, NKp46, NTPDase-1, OX40, OX40L, PD-1, PD-L1, PD-L2, PROM1, 5152, SIRPalpha, SISP1, SLC, SPG64, ST2, STEAP1, STEAP2, Syk kinase, STEAP1, TROP2, TACI, TDO, TGFBETA, T14, TIGIT, TIM3, TLR, TLR2, TLR4, TLR5, TLR9, TMEF1, TNFa, TNFRSF7, Tp55, TREM1, TSLP, TSLPR, TWEAK, VEGF, VISTA, Vstm3, or WUCAM. In some aspects, VL2 and VH2 specifically bind to A2AR, APRIL, ATPDase, BAFF, BAFFR, BCMA, BlyS, BTK, BTLA, B7DC, B7H1, B7H2, B7H3, B7H4, B7H5, B7H6, B7H7, B7RP1, B7-4, C3, C5, CCL2, CCL3, CCL4, CCL5, CCL7, CCL8, CCL11, CCL15, CCL17, CCL19, CCL20, CCL21, CCL25, CCR3, CCR4, CD16A, CD19, CD20, CD24, CD27, CD28, CD30, CD38, CD39, CD40, CD40L, CD47, CD52, CD70, CD80, CD86, CD123, CD133, CD137, CD137L, CD160, CD272, CEACAM5, CLEC9, CLEC91, CRTH2, CSF-1, CSF-2, CSF-3, CXCL1, CXCL2, CXCL4, CXCL12, CXCL13, CXCR3, cMet, CTLA4, DLL3, DLL4, DNGR-1, E-cadherin, EGFR, ENTPD1, EpCAM, FCER1, FCER1A, FCER2, FGFR, FLAP, FOLH1, Gi24, GITR, GITRL, GPR5, GP100, GPRC5D, HER2, HER3, ICOSL, ICOS, HHLA2, HMGB1, HVEM, IDO, IFNa, IgE, IGF1R, IL2Rbeta, IL1, ILIA, IL1B, IL1F10, IL2, IL4, IL4Ra, IL5, IL5R, IL6, IL7, IL7Ra, IL8, IL9, IL9R, IL10, rhILlO, IL12, IL13, IL13Ra1, IL13Ra2, IL15, IL17, IL17Rb, IL18, IL22, IL23, IL25, IL7, IL33, IL35, ITGB4, ITK, KIR, LAG3, LAMP1, leptin, LPFS2, MHC class II, MUC-1, MUC-16, NCR3LG1, NKG2D, NKp46, NTPDase-1, OX40, OX40L, PD-1, PD-L1, PD-L2, PROM1, 5152, SIRPalpha, SISP1, SLC, SPG64, ST2, STEAP1, STEAP2, Syk kinase, STEAP1, TROP2, TACI, TDO, TGFBETA, T14, TIGIT, TIM3, TLR, TLR2, TLR4, TLR5, TLR9, TMEF1, TNFa, TNFRSF7, Tp55, TREM1, TSLP, TSLPR, TWEAK, VEGF, VISTA, Vstm3, or WUCAM. In some aspects, VL4 and VH4 specifically bind to A2AR, APRIL, ATPDase, BAFF, BAFFR, BCMA, BlyS, BTK, BTLA, B7DC, B7H1, B7H2, B7H3, B7H4, B7H5, B7H6, B7H7, B7RP1, B7-4, C3, C5, CCL2, CCL3, CCL4, CCL5, CCL7, CCL8, CCL11, CCL15, CCL17, CCL19, CCL20, CCL21, CCL25, CCR3, CCR4, CD16A, CD19, CD20, CD24, CD27, CD28, CD30, CD38, CD39, CD40, CD40L, CD47, CD52, CD70, CD80, CD86, CD123, CD133, CD137, CD137L, CD160, CD272, CEACAM5, CLEC9, CLEC91, CRTH2, CSF-1, CSF-2, CSF-3, CXCL1, CXCL2, CXCL4, CXCL12, CXCL13, CXCR3, cMet, CTLA4, DLL3, DLL4, DNGR-1, E-cadherin, EGFR, ENTPD1, EpCAM, FCER1, FCER1A, FCER2, FGFR, FLAP, FOLH1, Gi24, GITR, GITRL, GPR5, GP100, GPRC5D, HER2, HER3, ICOSL, ICOS, HHLA2, HMGB1, HVEM, IDO, IFNa, IgE, IGF1R, IL2Rbeta, IL1, ILIA, IL1B, IL1F10, IL2, IL4, IL4Ra, IL5, IL5R, IL6, IL7, IL7Ra, IL8, IL9, IL9R, IL10, rhILlO, IL12, IL13, IL13Ra1, IL13Ra2, IL15, IL17, IL17Rb, IL18, IL22, IL23, IL25, IL7, IL33, IL35, ITGB4, ITK, KIR, LAG3, LAMP1, leptin, LPFS2, MHC class II, MUC-1, MUC-16, NCR3LG1, NKG2D, NKp46, NTPDase-1, OX40, OX40L, PD-1, PD-L1, PD-L2, PROM1, S152, SIRPalpha, SISP1, SLC, SPG64, ST2, STEAP1, STEAP2, Syk kinase, STEAP1, TROP2, TACI, TDO, TGFBETA, T14, TIGIT, TIM3, TLR, TLR2, TLR4, TLR5, TLR9, TMEF1, TNFa, TNFRSF7, Tp55, TREM1, TSLP, TSLPR, TWEAK, VEGF, VISTA, Vstm3, or WUCAM. The VL1, VL2, VL4, VH1, VH2, and/or VH4 may specifically bind to CD28, cMet, Trop2, CD20, or CD19; and VL3 and/or VH3 may specifically bind to CD3. For example, the antigen binding polypeptide complex may specifically bind CD28, CD3, cMet, and Trop2. For example, the antigen binding polypeptide complex may specifically bind CD28, CD3, CD19, and CD20. For example, VL1 and VH1 may specifically bind to CD28. For example, VL2 and VH2 may specifically bind to CD28. For example, VL4 and VH4 may specifically bind to CD28. For example, VL1 and VH1 may specifically bind to cMet. For example, VL2 and VH2 may specifically bind to cMet. For example, VL4 and VH4 may specifically bind to cMet. For example, VL1 and VH1 may specifically bind to Trop2. For example, VL2 and VH2 may specifically bind to Trop2. For example, VL4 and VH4 may specifically bind to Trop2. For example, VL1 and VH1 may specifically bind to CD19. For example, VL2 and VH2 may specifically bind to CD19. For example, VL4 and VH4 may specifically bind to CD19. For example, VL1 and VH1 may specifically bind to CD20. For example, VL2 and VH2 may specifically bind to CD20. For example, VL4 and VH4 may specifically bind to CD20.

In some aspects, the invention is directed to an antigen binding polypeptide complex comprising a first polypeptide and a second polypeptide; wherein the first polypeptide has a structure represented by VL1-L1-VL2-L2-VH2-L3-VH1-L4-CH1-L5-Fc; VH1-L6-VH2-L7-VL2-L8-VL1-L9-CH1-L10-Fc; VL1-L11-VL2-L12-VH2-L13-VH1-L14-CL-L15-Fc; VH1-L16-VH2-L17-VL2-L18-VL1-L19-CL-L20-Fc; VL1-L21-VL2-L22-VH2-L23-VH1-L24-CH1-L25-CL-L26-Fc; VH1-L27-VH2-L28-VL2-L29-VL1-L30-CH1-L31-CL-L32-Fc; VL1-L33-VL2-L34-VH2-L35-VH1-L36-CL-L37-CH1-L38-Fc; or VH1-L39-VH2-L40-VL2-L41-VL1-L42-CL-L43-CH1-L44-Fc; wherein the second polypeptide has a structure represented by VL3-L45-VL4-L46-VH4-L47-VH3-L48-CH1-L49-Fc; VH3-L50-VH4-L51-VL4-L52-VL3-L53-CH1-L54-Fc; VL3-L55-VL4-L56-VH4-L57-VH3-L58-CL-L59-Fc; VH3-L60-VH4-L61-VL4-L62-VL3-L63-CL-L64-Fc; VL3-L65-VL4-L66-VH4-L67-VH3-L68-CH1-L69-CL-L70-Fc; VH3-L71-VH4-L72-VL4-L73-VL3-L74-CH1-L75-CL-L76-Fc; VL3-L77-VL4-L78-VH4-L79-VH3-L80-CL-L81-CH1-L82-Fc; or VH3-L83-VH4-L84-VL4-L85-VL3-L86-CL-L87-CH1-L88-Fc; wherein VL1 is a first immunoglobulin light chain variable region that specifically binds to CD28; VL2 is a second immunoglobulin light chain variable region that specifically binds to A2AR, APRIL, ATPDase, BAFF, BAFFR, BCMA, BlyS, BTK, BTLA, B7DC, B7H1, B7H2, B7H3, B7H4, B7H5, B7H6, B7H7, B7RP1, B7-4, C3, C5, CCL2, CCL3, CCL4, CCL5, CCL7, CCL8, CCL11, CCL15, CCL17, CCL19, CCL20, CCL21, CCL25, CCR3, CCR4, CD16A, CD19, CD20, CD24, CD27, CD28, CD30, CD38, CD39, CD40, CD40L, CD47, CD52, CD70, CD80, CD86, CD123, CD133, CD137, CD137L, CD160, CD272, CEACAM5, CLEC9, CLEC91, CRTH2, CSF-1, CSF-2, CSF-3, CXCL1, CXCL2, CXCL4, CXCL12, CXCL13, CXCR3, cMet, CTLA4, DLL3, DLL4, DNGR-1, E-cadherin, EGFR, ENTPD1, EpCAM, FCER1, FCER1A, FCER2, FGFR, FLAP, FOLH1, Gi24, GITR, GITRL, GPR5, GP100, GPRC5D, HER2, HER3, ICOSL, ICOS, HHLA2, HMGB1, HVEM, IDO, IFNa, IgE, IGF1R, IL2Rbeta, IL1, ILIA, IL1B, IL1F10, IL2, IL4, IL4Ra, IL5, IL5R, IL6, IL7, IL7Ra, IL8, IL9, IL9R, IL10, rhILlO, IL12, IL13, IL13Ra1, IL13Ra2, IL15, IL17, IL17Rb, IL18, IL22, IL23, IL25, IL7, IL33, IL35, ITGB4, ITK, KIR, LAG3, LAMP1, leptin, LPFS2, MHC class II, MUC-1, MUC-16, NCR3LG1, NKG2D, NKp46, NTPDase-1, OX40, OX40L, PD-1, PD-L1, PD-L2, PROM1, S152, SIRPalpha, SISP1, SLC, SPG64, ST2, STEAP1, STEAP2, Syk kinase, STEAP1, TROP2, TACI, TDO, TGFBETA, T14, TIGIT, TIM3, TLR, TLR2, TLR4, TLR5, TLR9, TMEF1, TNFa, TNFRSF7, Tp55, TREM1, TSLP, TSLPR, TWEAK, VEGF, VISTA, Vstm3, or WUCAM; VL3 is a third immunoglobulin light chain variable region that specifically binds to CD3; VL4 is a fourth immunoglobulin light chain variable region that specifically binds to A2AR, APRIL, ATPDase, BAFF, BAFFR, BCMA, BlyS, BTK, BTLA, B7DC, B7H1, B7H2, B7H3, B7H4, B7H5, B7H6, B7H7, B7RP1, B7-4, C3, C5, CCL2, CCL3, CCL4, CCL5, CCL7, CCL8, CCL11, CCL15, CCL17, CCL19, CCL20, CCL21, CCL25, CCR3, CCR4, CD16A, CD19, CD20, CD24, CD27, CD28, CD30, CD38, CD39, CD40, CD40L, CD47, CD52, CD70, CD80, CD86, CD123, CD133, CD137, CD137L, CD160, CD272, CEACAM5, CLEC9, CLEC91, CRTH2, CSF-1, CSF-2, CSF-3, CXCL1, CXCL2, CXCL4, CXCL12, CXCL13, CXCR3, cMet, CTLA4, DLL3, DLL4, DNGR-1, E-cadherin, EGFR, ENTPD1, EpCAM, FCER1, FCER1A, FCER2, FGFR, FLAP, FOLH1, Gi24, GITR, GITRL, GPR5, GP100, GPRC5D, HER2, HER3, ICOSL, ICOS, HHLA2, HMGB1, HVEM, IDO, IFNa, IgE, IGF1R, IL2Rbeta, IL1, ILIA, IL1B, IL1F10, IL2, IL4, IL4Ra, IL5, IL5R, IL6, IL7, IL7Ra, IL8, IL9, IL9R, IL10, rhILlO, IL12, IL13, IL13Ra1, IL13Ra2, IL15, IL17, IL17Rb, IL18, IL22, IL23, IL25, IL7, IL33, IL35, ITGB4, ITK, KIR, LAG3, LAMP1, leptin, LPFS2, MHC class II, MUC-1, MUC-16, NCR3LG1, NKG2D, NKp46, NTPDase-1, OX40, OX40L, PD-1, PD-L1, PD-L2, PROM1, S152, SIRPalpha, SISP1, SLC, SPG64, ST2, STEAP1, STEAP2, Syk kinase, STEAP1, TROP2, TACI, TDO, TGFBETA, T14, TIGIT, TIM3, TLR, TLR2, TLR4, TLR5, TLR9, TMEF1, TNFa, TNFRSF7, Tp55, TREM1, TSLP, TSLPR, TWEAK, VEGF, VISTA, Vstm3, or WUCAM; VH1 is a first immunoglobulin heavy chain variable region that specifically binds to CD28; VH2 is a second immunoglobulin heavy chain variable region that specifically binds to A2AR, APRIL, ATPDase, BAFF, BAFFR, BCMA, BlyS, BTK, BTLA, B7DC, B7H1, B7H2, B7H3, B7H4, B7H5, B7H6, B7H7, B7RP1, B7-4, C3, C5, CCL2, CCL3, CCL4, CCL5, CCL7, CCL8, CCL11, CCL15, CCL17, CCL19, CCL20, CCL21, CCL25, CCR3, CCR4, CD16A, CD19, CD20, CD24, CD27, CD28, CD30, CD38, CD39, CD40, CD40L, CD47, CD52, CD70, CD80, CD86, CD123, CD133, CD137, CD137L, CD160, CD272, CEACAM5, CLEC9, CLEC91, CRTH2, CSF-1, CSF-2, CSF-3, CXCL1, CXCL2, CXCL4, CXCL12, CXCL13, CXCR3, cMet, CTLA4, DLL3, DLL4, DNGR-1, E-cadherin, EGFR, ENTPD1, EpCAM, FCER1, FCER1A, FCER2, FGFR, FLAP, FOLH1, Gi24, GITR, GITRL, GPR5, GP100, GPRC5D, HER2, HER3, ICOSL, ICOS, HHLA2, HMGB1, HVEM, IDO, IFNa, IgE, IGF1R, IL2Rbeta, IL1, ILIA, IL1B, IL1F10, IL2, IL4, IL4Ra, IL5, IL5R, IL6, IL7, IL7Ra, IL8, IL9, IL9R, IL10, rhILlO, IL12, IL13, IL13Ra1, IL13Ra2, IL15, IL17, IL17Rb, IL18, IL22, IL23, IL25, IL7, IL33, IL35, ITGB4, ITK, KIR, LAG3, LAMP1, leptin, LPFS2, MHC class II, MUC-1, MUC-16, NCR3LG1, NKG2D, NKp46, NTPDase-1, OX40, OX40L, PD-1, PD-L1, PD-L2, PROM1, S152, SIRPalpha, SISP1, SLC, SPG64, ST2, STEAP1, STEAP2, Syk kinase, STEAP1, TROP2, TACI, TDO, TGFBETA, T14, TIGIT, TIM3, TLR, TLR2, TLR4, TLR5, TLR9, TMEF1, TNFa, TNFRSF7, Tp55, TREM1, TSLP, TSLPR, TWEAK, VEGF, VISTA, Vstm3, or WUCAM; VH3 is a third immunoglobulin heavy chain variable region that specifically binds to CD3; VH4 is a fourth immunoglobulin heavy chain variable region that specifically binds to A2AR, APRIL, ATPDase, BAFF, BAFFR, BCMA, BlyS, BTK, BTLA, B7DC, B7H1, B7H2, B7H3, B7H4, B7H5, B7H6, B7H7, B7RP1, B7-4, C3, C5, CCL2, CCL3, CCL4, CCL5, CCL7, CCL8, CCL11, CCL15, CCL17, CCL19, CCL20, CCL21, CCL25, CCR3, CCR4, CD16A, CD19, CD20, CD24, CD27, CD28, CD30, CD38, CD39, CD40, CD40L, CD47, CD52, CD70, CD80, CD86, CD123, CD133, CD137, CD137L, CD160, CD272, CEACAM5, CLEC9, CLEC91, CRTH2, CSF-1, CSF-2, CSF-3, CXCL1, CXCL2, CXCL4, CXCL12, CXCL13, CXCR3, cMet, CTLA4, DLL3, DLL4, DNGR-1, E-cadherin, EGFR, ENTPD1, EpCAM, FCER1, FCER1A, FCER2, FGFR, FLAP, FOLH1, Gi24, GITR, GITRL, GPR5, GP100, GPRC5D, HER2, HER3, ICOSL, ICOS, HHLA2, HMGB1, HVEM, IDO, IFNa, IgE, IGF1R, IL2Rbeta, IL1, ILIA, IL1B, IL1F10, IL2, IL4, IL4Ra, IL5, IL5R, IL6, IL7, IL7Ra, IL8, IL9, IL9R, IL10, rhILlO, IL12, IL13, IL13Ra1, IL13Ra2, IL15, IL17, IL17Rb, IL18, IL22, IL23, IL25, IL7, IL33, IL35, ITGB4, ITK, KIR, LAG3, LAMP1, leptin, LPFS2, MHC class II, MUC-1, MUC-16, NCR3LG1, NKG2D, NKp46, NTPDase-1, OX40, OX40L, PD-1, PD-L1, PD-L2, PROM1, S152, SIRPalpha, SISP1, SLC, SPG64, ST2, STEAP1, STEAP2, Syk kinase, STEAP1, TROP2, TACI, TDO, TGFBETA, T14, TIGIT, TIM3, TLR, TLR2, TLR4, TLR5, TLR9, TMEF1, TNFa, TNFRSF7, Tp55, TREM1, TSLP, TSLPR, TWEAK, VEGF, VISTA, Vstm3, or WUCAM; Fc is a region comprising an immunoglobulin heavy chain constant region 2 (CH2), an immunoglobulin heavy chain constant region 3 (CH3), and optionally, an immunoglobulin hinge; CH1 is an immunoglobulin heavy chain constant region 1; CL is an immunoglobulin light chain constant region; and L1-L88 are amino acid linkers. In some aspects, the first polypeptide has a structure represented by VL1-L1-VL2-L2-VH2-L3-VH1-L4-CH1-L5-Fc; VH1-L6-VH2-L7-VL2-L8-VL1-L9-CH1-L10-Fc; VL1-L11-VL2-L12-VH2-L13-VH1-L14-CL-L15-Fc; VH1-L16-VH2-L17-VL2-L18-VL1-L19-CL-L20-Fc; VL1-L21-VL2-L22-VH2-L23-VH1-L24-CH1-L25-CL-L26-Fc; VH1-L27-VH2-L28-VL2-L29-VL1-L30-CH1-L31-CL-L32-Fc; VL1-L33-VL2-L34-VH2-L35-VH1-L36-CL-L37-CH1-L38-Fc; or VH1-L39-VH2-L40-VL2-L41-VL1-L42-CL-L43-CH1-L44-Fc and the second polypeptide has a structure represented by VL3-L45-VL4-L46-VH4-L47-VH3-L48-CH1-L49-Fc. In some aspects, the first polypeptide has a structure represented by VL1-L1-VL2-L2-VH2-L3-VH1-L4-CH1-L5-Fc; VH1-L6-VH2-L7-VL2-L8-VL1-L9-CH1-L10-Fc; VL1-L11-VL2-L12-VH2-L13-VH1-L14-CL-L15-Fc; VH1-L16-VH2-L17-VL2-L18-VL1-L19-CL-L20-Fc; VL1-L21-VL2-L22-VH2-L23-VH1-L24-CH1-L25-CL-L26-Fc; VH1-L27-VH2-L28-VL2-L29-VL1-L30-CH1-L31-CL-L32-Fc; VL1-L33-VL2-L34-VH2-L35-VH1-L36-CL-L37-CH1-L38-Fc; or VH1-L39-VH2-L40-VL2-L41-VL1-L42-CL-L43-CH1-L44-Fc and the second polypeptide has a structure represented by VH3-L50-VH4-L51-VL4-L52-VL3-L53-CH1-L54-Fc. In some aspects, the first polypeptide has a structure represented by VL1-L1-VL2-L2-VH2-L3-VH1-L4-CH1-L5-Fc; VH1-L6-VH2-L7-VL2-L8-VL1-L9-CH1-L10-Fc; VL1-L11-VL2-L12-VH2-L13-VH1-L14-CL-L15-Fc; VH1-L16-VH2-L17-VL2-L18-VL1-L19-CL-L20-Fc; VL1-L21-VL2-L22-VH2-L23-VH1-L24-CH1-L25-CL-L26-Fc; VH1-L27-VH2-L28-VL2-L29-VL1-L30-CH1-L31-CL-L32-Fc; VL1-L33-VL2-L34-VH2-L35-VH1-L36-CL-L37-CH1-L38-Fc; or VH1-L39-VH2-L40-VL2-L41-VL1-L42-CL-L43-CH1-L44-Fc and the second polypeptide has a structure represented by VL3-L55-VL4-L56-VH4-L57-VH3-L58-CL-L59-Fc. In some aspects, the first polypeptide has a structure represented by VL1-L1-VL2-L2-VH2-L3-VH1-L4-CH1-L5-Fc; VH1-L6-VH2-L7-VL2-L8-VL1-L9-CH1-L10-Fc; VL1-L11-VL2-L12-VH2-L13-VH1-L14-CL-L15-Fc; VH1-L16-VH2-L17-VL2-L18-VL1-L19-CL-L20-Fc; VL1-L21-VL2-L22-VH2-L23-VH1-L24-CH1-L25-CL-L26-Fc; VH1-L27-VH2-L28-VL2-L29-VL1-L30-CH1-L31-CL-L32-Fc; VL1-L33-VL2-L34-VH2-L35-VH1-L36-CL-L37-CH1-L38-Fc; or VH1-L39-VH2-L40-VL2-L41-VL1-L42-CL-L43-CH1-L44-Fc and the second polypeptide has a structure represented by VH3-L60-VH4-L61-VL4-L62-VL3-L63-CL-L64-Fc. In some aspects, the first polypeptide has a structure represented by VL1-L1-VL2-L2-VH2-L3-VH1-L4-CH1-L5-Fc; VH1-L6-VH2-L7-VL2-L8-VL1-L9-CH1-L10-Fc; VL1-L11-VL2-L12-VH2-L13-VH1-L14-CL-L15-Fc; VH1-L16-VH2-L17-VL2-L18-VL1-L19-CL-L20-Fc; VL1-L21-VL2-L22-VH2-L23-VH1-L24-CH1-L25-CL-L26-Fc; VH1-L27-VH2-L28-VL2-L29-VL1-L30-CH1-L31-CL-L32-Fc; VL1-L33-VL2-L34-VH2-L35-VH1-L36-CL-L37-CH1-L38-Fc; or VH1-L39-VH2-L40-VL2-L41-VL1-L42-CL-L43-CH1-L44-Fc and the second polypeptide has a structure represented by VL3-L65-VL4-L66-VH4-L67-VH3-L68-CH1-L69-CL-L70-Fc. In some aspects, the first polypeptide has a structure represented by VL1-L1-VL2-L2-VH2-L3-VH1-L4-CH1-L5-Fc; VH1-L6-VH2-L7-VL2-L8-VL1-L9-CH1-L10-Fc; VL1-L11-VL2-L12-VH2-L13-VH1-L14-CL-L15-Fc; VH1-L16-VH2-L17-VL2-L18-VL1-L19-CL-L20-Fc; VL1-L21-VL2-L22-VH2-L23-VH1-L24-CH1-L25-CL-L26-Fc; VH1-L27-VH2-L28-VL2-L29-VL1-L30-CH1-L31-CL-L32-Fc; VL1-L33-VL2-L34-VH2-L35-VH1-L36-CL-L37-CH1-L38-Fc; or VH1-L39-VH2-L40-VL2-L41-VL1-L42-CL-L43-CH1-L44-Fc and the second polypeptide has a structure represented by VH3-L71-VH4-L72-VL4-L73-VL3-L74-CH1-L75-CL-L76-Fc. In some aspects, the first polypeptide has a structure represented by VL1-L1-VL2-L2-VH2-L3-VH1-L4-CH1-L5-Fc; VH1-L6-VH2-L7-VL2-L8-VL1-L9-CH1-L10-Fc; VL1-L11-VL2-L12-VH2-L13-VH1-L14-CL-L15-Fc; VH1-L16-VH2-L17-VL2-L18-VL1-L19-CL-L20-Fc; VL1-L21-VL2-L22-VH2-L23-VH1-L24-CH1-L25-CL-L26-Fc; VH1-L27-VH2-L28-VL2-L29-VL1-L30-CH1-L31-CL-L32-Fc; VL1-L33-VL2-L34-VH2-L35-VH1-L36-CL-L37-CH1-L38-Fc; or VH1-L39-VH2-L40-VL2-L41-VL1-L42-CL-L43-CH1-L44-Fc and the second polypeptide has a structure represented by VL3-L77-VL4-L78-VH4-L79-VH3-L80-CL-L81-CH1-L82-Fc. In some aspects, the first polypeptide has a structure represented by VL1-L1-VL2-L2-VH2-L3-VH1-L4-CH1-L5-Fc; VH1-L6-VH2-L7-VL2-L8-VL1-L9-CH1-L10-Fc; VL1-L11-VL2-L12-VH2-L13-VH1-L14-CL-L15-Fc; VH1-L16-VH2-L17-VL2-L18-VL1-L19-CL-L20-Fc; VL1-L21-VL2-L22-VH2-L23-VH1-L24-CH1-L25-CL-L26-Fc; VH1-L27-VH2-L28-VL2-L29-VL1-L30-CH1-L31-CL-L32-Fc; VL1-L33-VL2-L34-VH2-L35-VH1-L36-CL-L37-CH1-L38-Fc; or VH1-L39-VH2-L40-VL2-L41-VL1-L42-CL-L43-CH1-L44-Fc and the second polypeptide has a structure represented by VH3-L83-VH4-L84-VL4-L85-VL3-L86-CL-L87-CH1-L88-Fc. In some aspects, VL2 and VH2 specifically bind to A2AR, APRIL, ATPDase, BAFF, BAFFR, BCMA, BlyS, BTK, BTLA, B7DC, B7H1, B7H2, B7H3, B7H4, B7H5, B7H6, B7H7, B7RP1, B7-4, C3, C5, CCL2, CCL3, CCL4, CCL5, CCL7, CCL8, CCL11, CCL15, CCL17, CCL19, CCL20, CCL21, CCL25, CCR3, CCR4, CD16A, CD19, CD20, CD24, CD27, CD28, CD30, CD38, CD39, CD40, CD40L, CD47, CD52, CD70, CD80, CD86, CD123, CD133, CD137, CD137L, CD160, CD272, CEACAM5, CLEC9, CLEC91, CRTH2, CSF-1, CSF-2, CSF-3, CXCL1, CXCL2, CXCL4, CXCL12, CXCL13, CXCR3, cMet, CTLA4, DLL3, DLL4, DNGR-1, E-cadherin, EGFR, ENTPD1, EpCAM, FCER1, FCER1A, FCER2, FGFR, FLAP, FOLH1, Gi24, GITR, GITRL, GPR5, GP100, GPRC5D, HER2, HER3, ICOSL, ICOS, HHLA2, HMGB1, HVEM, IDO, IFNa, IgE, IGF1R, IL2Rbeta, IL1, ILIA, IL1B, IL1F10, IL2, IL4, IL4Ra, IL5, IL5R, IL6, IL7, IL7Ra, IL8, IL9, IL9R, IL10, rhILlO, IL12, IL13, IL13Ra1, IL13Ra2, IL15, IL17, IL17Rb, IL18, IL22, IL23, IL25, IL7, IL33, IL35, ITGB4, ITK, KIR, LAG3, LAMP1, leptin, LPFS2, MHC class II, MUC-1, MUC-16, NCR3LG1, NKG2D, NKp46, NTPDase-1, OX40, OX40L, PD-1, PD-L1, PD-L2, PROM1, S152, SIRPalpha, SISP1, SLC, SPG64, ST2, STEAP1, STEAP2, Syk kinase, STEAP1, TROP2, TACI, TDO, TGFBETA, T14, TIGIT, TIM3, TLR, TLR2, TLR4, TLR5, TLR9, TMEF1, TNFa, TNFRSF7, Tp55, TREM1, TSLP, TSLPR, TWEAK, VEGF, VISTA, Vstm3, or WUCAM. In some aspects, VL4 and VH4 specifically bind to A2AR, APRIL, ATPDase, BAFF, BAFFR, BCMA, BlyS, BTK, BTLA, B7DC, B7H1, B7H2, B7H3, B7H4, B7H5, B7H6, B7H7, B7RP1, B7-4, C3, C5, CCL2, CCL3, CCL4, CCL5, CCL7, CCL8, CCL11, CCL15, CCL17, CCL19, CCL20, CCL21, CCL25, CCR3, CCR4, CD16A, CD19, CD20, CD24, CD27, CD28, CD30, CD38, CD39, CD40, CD40L, CD47, CD52, CD70, CD80, CD86, CD123, CD133, CD137, CD137L, CD160, CD272, CEACAM5, CLEC9, CLEC91, CRTH2, CSF-1, CSF-2, CSF-3, CXCL1, CXCL2, CXCL4, CXCL12, CXCL13, CXCR3, cMet, CTLA4, DLL3, DLL4, DNGR-1, E-cadherin, EGFR, ENTPD1, EpCAM, FCER1, FCER1A, FCER2, FGFR, FLAP, FOLH1, Gi24, GITR, GITRL, GPR5, GP100, GPRC5D, HER2, HER3, ICOSL, ICOS, HHLA2, HMGB1, HVEM, IDO, IFNa, IgE, IGF1R, IL2Rbeta, IL1, ILIA, IL1B, IL1F10, IL2, IL4, IL4Ra, IL5, IL5R, IL6, IL7, IL7Ra, IL8, IL9, IL9R, IL10, rhILlO, IL12, IL13, IL13Ra1, IL13Ra2, IL15, IL17, IL17Rb, IL18, IL22, IL23, IL25, IL7, IL33, IL35, ITGB4, ITK, KIR, LAG3, LAMP1, leptin, LPFS2, MHC class II, MUC-1, MUC-16, NCR3LG1, NKG2D, NKp46, NTPDase-1, OX40, OX40L, PD-1, PD-L1, PD-L2, PROM1, S152, SIRPalpha, SISP1, SLC, SPG64, ST2, STEAP1, STEAP2, Syk kinase, STEAP1, TROP2, TACI, TDO, TGFBETA, T14, TIGIT, TIM3, TLR, TLR2, TLR4, TLR5, TLR9, TMEF1, TNFa, TNFRSF7, Tp55, TREM1, TSLP, TSLPR, TWEAK, VEGF, VISTA, Vstm3, or WUCAM. The VL1 and/or VH1 may specifically bind to CD28; VL2, VL4, VH2, and/or VH4 may specifically bind to cMet, Trop2, CD20, or CD19; and VL3 and/or VH3 may specifically bind to CD3. For example, the antigen binding polypeptide complex may specifically bind CD28, CD3, cMet, and Trop2. For example, the antigen binding polypeptide complex may specifically bind CD28, CD3, CD19, and CD20. For example, VL2 and VH2 may specifically bind to cMet. For example, VL4 and VH4 may specifically bind to cMet. For example, VL2 and VH2 may specifically bind to Trop2. For example, VL4 and VH4 may specifically bind to Trop2. For example, VL2 and VH2 may specifically bind to CD19. For example, VL4 and VH4 may specifically bind to CD19. For example, VL2 and VH2 may specifically bind to CD20. For example, VL4 and VH4 may specifically bind to CD20.

In some aspects, the invention is directed to an antigen binding polypeptide complex comprising a first polypeptide and a second polypeptide; wherein the first polypeptide has a structure represented by VL1-L1-VL2-L2-VH2-L3-VH1-L4-CH1-L5-Fc; VH1-L6-VH2-L7-VL2-L8-VL1-L9-CH1-L10-Fc; VL1-L11-VL2-L12-VH2-L13-VH1-L14-CL-L15-Fc; VH1-L16-VH2-L17-VL2-L18-VL1-L19-CL-L20-Fc; VL1-L21-VL2-L22-VH2-L23-VH1-L24-CH1-L25-CL-L26-Fc; VH1-L27-VH2-L28-VL2-L29-VL1-L30-CH1-L31-CL-L32-Fc; VL1-L33-VL2-L34-VH2-L35-VH1-L36-CL-L37-CH1-L38-Fc; or VH1-L39-VH2-L40-VL2-L41-VL1-L42-CL-L43-CH1-L44-Fc; wherein the second polypeptide has a structure represented by VL3-L45-VL4-L46-VH4-L47-VH3-L48-CH1-L49-Fc; VH3-L50-VH4-L51-VL4-L52-VL3-L53-CH1-L54-Fc; VL3-L55-VL4-L56-VH4-L57-VH3-L58-CL-L59-Fc; VH3-L60-VH4-L61-VL4-L62-VL3-L63-CL-L64-Fc; VL3-L65-VL4-L66-VH4-L67-VH3-L68-CH1-L69-CL-L70-Fc; VH3-L71-VH4-L72-VL4-L73-VL3-L74-CH1-L75-CL-L76-Fc; VL3-L77-VL4-L78-VH4-L79-VH3-L80-CL-L81-CH1-L82-Fc; or VH3-L83-VH4-L84-VL4-L85-VL3-L86-CL-L87-CH1-L88-Fc; wherein VL1 is a first immunoglobulin light chain variable region that specifically binds to A2AR, APRIL, ATPDase, BAFF, BAFFR, BCMA, BlyS, BTK, BTLA, B7DC, B7H1, B7H2, B7H3, B7H4, B7H5, B7H6, B7H7, B7RP1, B7-4, C3, C5, CCL2, CCL3, CCL4, CCL5, CCL7, CCL8, CCL11, CCL15, CCL17, CCL19, CCL20, CCL21, CCL25, CCR3, CCR4, CD16A, CD19, CD20, CD24, CD27, CD28, CD30, CD38, CD39, CD40, CD40L, CD47, CD52, CD70, CD80, CD86, CD123, CD133, CD137, CD137L, CD160, CD272, CEACAM5, CLEC9, CLEC91, CRTH2, CSF-1, CSF-2, CSF-3, CXCL1, CXCL2, CXCL4, CXCL12, CXCL13, CXCR3, cMet, CTLA4, DLL3, DLL4, DNGR-1, E-cadherin, EGFR, ENTPD1, EpCAM, FCER1, FCER1A, FCER2, FGFR, FLAP, FOLH1, Gi24, GITR, GITRL, GPR5, GP100, GPRC5D, HER2, HER3, ICOSL, ICOS, HHLA2, HMGB1, HVEM, IDO, IFNa, IgE, IGF1R, IL2Rbeta, IL1, ILIA, IL1B, IL1F10, IL2, IL4, IL4Ra, IL5, IL5R, IL6, IL7, IL7Ra, IL8, IL9, IL9R, IL10, rhILlO, IL12, IL13, IL13Ra1, IL13Ra2, IL15, IL17, IL17Rb, IL18, IL22, IL23, IL25, IL7, IL33, IL35, ITGB4, ITK, KIR, LAG3, LAMP1, leptin, LPFS2, MHC class II, MUC-1, MUC-16, NCR3LG1, NKG2D, NKp46, NTPDase-1, OX40, OX40L, PD-1, PD-L1, PD-L2, PROM1, S152, SIRPalpha, SISP1, SLC, SPG64, ST2, STEAP1, STEAP2, Syk kinase, STEAP1, TROP2, TACI, TDO, TGFBETA, T14, TIGIT, TIM3, TLR, TLR2, TLR4, TLR5, TLR9, TMEF1, TNFa, TNFRSF7, Tp55, TREM1, TSLP, TSLPR, TWEAK, VEGF, VISTA, Vstm3, or WUCAM; VL2 is a second immunoglobulin light chain variable region that specifically binds to CD28; VL3 is a third immunoglobulin light chain variable region that specifically binds to CD3; VL4 is a fourth immunoglobulin light chain variable region that specifically binds to A2AR, APRIL, ATPDase, BAFF, BAFFR, BCMA, BlyS, BTK, BTLA, B7DC, B7H1, B7H2, B7H3, B7H4, B7H5, B7H6, B7H7, B7RP1, B7-4, C3, C5, CCL2, CCL3, CCL4, CCL5, CCL7, CCL8, CCL11, CCL15, CCL17, CCL19, CCL20, CCL21, CCL25, CCR3, CCR4, CD16A, CD19, CD20, CD24, CD27, CD28, CD30, CD38, CD39, CD40, CD40L, CD47, CD52, CD70, CD80, CD86, CD123, CD133, CD137, CD137L, CD160, CD272, CEACAM5, CLEC9, CLEC91, CRTH2, CSF-1, CSF-2, CSF-3, CXCL1, CXCL2, CXCL4, CXCL12, CXCL13, CXCR3, cMet, CTLA4, DLL3, DLL4, DNGR-1, E-cadherin, EGFR, ENTPD1, EpCAM, FCER1, FCER1A, FCER2, FGFR, FLAP, FOLH1, Gi24, GITR, GITRL, GPR5, GP100, GPRC5D, HER2, HER3, ICOSL, ICOS, HHLA2, HMGB1, HVEM, IDO, IFNa, IgE, IGF1R, IL2Rbeta, IL1, ILIA, IL1B, IL1F10, IL2, IL4, IL4Ra, IL5, IL5R, IL6, IL7, IL7Ra, IL8, IL9, IL9R, IL10, rhILlO, IL12, IL13, IL13Ra1, IL13Ra2, IL15, IL17, IL17Rb, IL18, IL22, IL23, IL25, IL7, IL33, IL35, ITGB4, ITK, KIR, LAG3, LAMP1, leptin, LPFS2, MHC class II, MUC-1, MUC-16, NCR3LG1, NKG2D, NKp46, NTPDase-1, OX40, OX40L, PD-1, PD-L1, PD-L2, PROM1, S152, SIRPalpha, SISP1, SLC, SPG64, ST2, STEAP1, STEAP2, Syk kinase, STEAP1, TROP2, TACI, TDO, TGFBETA, T14, TIGIT, TIM3, TLR, TLR2, TLR4, TLR5, TLR9, TMEF1, TNFa, TNFRSF7, Tp55, TREM1, TSLP, TSLPR, TWEAK, VEGF, VISTA, Vstm3, or WUCAM; VH1 is a first immunoglobulin heavy chain variable region that specifically binds to A2AR, APRIL, ATPDase, BAFF, BAFFR, BCMA, BlyS, BTK, BTLA, B7DC, B7H1, B7H2, B7H3, B7H4, B7H5, B7H6, B7H7, B7RP1, B7-4, C3, C5, CCL2, CCL3, CCL4, CCL5, CCL7, CCL8, CCL11, CCL15, CCL17, CCL19, CCL20, CCL21, CCL25, CCR3, CCR4, CD16A, CD19, CD20, CD24, CD27, CD28, CD30, CD38, CD39, CD40, CD40L, CD47, CD52, CD70, CD80, CD86, CD123, CD133, CD137, CD137L, CD160, CD272, CEACAM5, CLEC9, CLEC91, CRTH2, CSF-1, CSF-2, CSF-3, CXCL1, CXCL2, CXCL4, CXCL12, CXCL13, CXCR3, cMet, CTLA4, DLL3, DLL4, DNGR-1, E-cadherin, EGFR, ENTPD1, EpCAM, FCER1, FCER1A, FCER2, FGFR, FLAP, FOLH1, Gi24, GITR, GITRL, GPR5, GP100, GPRC5D, HER2, HER3, ICOSL, ICOS, HHLA2, HMGB1, HVEM, IDO, IFNa, IgE, IGF1R, IL2Rbeta, IL1, ILIA, IL1B, IL1F10, IL2, IL4, IL4Ra, IL5, IL5R, IL6, IL7, IL7Ra, IL8, IL9, IL9R, IL10, rhILlO, IL12, IL13, IL13Ra1, IL13Ra2, IL15, IL17, IL17Rb, IL18, IL22, IL23, IL25, IL7, IL33, IL35, ITGB4, ITK, KIR, LAG3, LAMP1, leptin, LPFS2, MHC class II, MUC-1, MUC-16, NCR3LG1, NKG2D, NKp46, NTPDase-1, OX40, OX40L, PD-1, PD-L1, PD-L2, PROM1, 5152, SIRPalpha, SISP1, SLC, SPG64, ST2, STEAP1, STEAP2, Syk kinase, STEAP1, TROP2, TACI, TDO, TGFBETA, T14, TIGIT, TIM3, TLR, TLR2, TLR4, TLR5, TLR9, TMEF1, TNFa, TNFRSF7, Tp55, TREM1, TSLP, TSLPR, TWEAK, VEGF, VISTA, Vstm3, or WUCAM; VH2 is a second immunoglobulin heavy chain variable region that specifically binds to CD28; VH3 is a third immunoglobulin heavy chain variable region that specifically binds to CD3; VH4 is a fourth immunoglobulin heavy chain variable region that specifically binds to A2AR, APRIL, ATPDase, BAFF, BAFFR, BCMA, BlyS, BTK, BTLA, B7DC, B7H1, B7H2, B7H3, B7H4, B7H5, B7H6, B7H7, B7RP1, B7-4, C3, C5, CCL2, CCL3, CCL4, CCL5, CCL7, CCL8, CCL11, CCL15, CCL17, CCL19, CCL20, CCL21, CCL25, CCR3, CCR4, CD16A, CD19, CD20, CD24, CD27, CD28, CD30, CD38, CD39, CD40, CD40L, CD47, CD52, CD70, CD80, CD86, CD123, CD133, CD137, CD137L, CD160, CD272, CEACAM5, CLEC9, CLEC91, CRTH2, CSF-1, CSF-2, CSF-3, CXCL1, CXCL2, CXCL4, CXCL12, CXCL13, CXCR3, cMet, CTLA4, DLL3, DLL4, DNGR-1, E-cadherin, EGFR, ENTPD1, EpCAM, FCER1, FCER1A, FCER2, FGFR, FLAP, FOLH1, Gi24, GITR, GITRL, GPR5, GP100, GPRC5D, HER2, HER3, ICOSL, ICOS, HHLA2, HMGB1, HVEM, IDO, IFNa, IgE, IGF1R, IL2Rbeta, IL1, ILIA, IL1B, IL1F10, IL2, IL4, IL4Ra, IL5, IL5R, IL6, IL7, IL7Ra, IL8, IL9, IL9R, IL10, rhILlO, IL12, IL13, IL13Ra1, IL13Ra2, IL15, IL17, IL17Rb, IL18, IL22, IL23, IL25, IL7, IL33, IL35, ITGB4, ITK, KIR, LAG3, LAMP1, leptin, LPFS2, MHC class II, MUC-1, MUC-16, NCR3LG1, NKG2D, NKp46, NTPDase-1, OX40, OX40L, PD-1, PD-L1, PD-L2, PROM1, S152, SIRPalpha, SISP1, SLC, SPG64, ST2, STEAP1, STEAP2, Syk kinase, STEAP1, TROP2, TACI, TDO, TGFBETA, T14, TIGIT, TIM3, TLR, TLR2, TLR4, TLR5, TLR9, TMEF1, TNFa, TNFRSF7, Tp55, TREM1, TSLP, TSLPR, TWEAK, VEGF, VISTA, Vstm3, or WUCAM; Fc is a region comprising an immunoglobulin heavy chain constant region 2 (CH2), an immunoglobulin heavy chain constant region 3 (CH3), and optionally, an immunoglobulin hinge; CH1 is an immunoglobulin heavy chain constant region 1; CL is an immunoglobulin light chain constant region; and L1-L88 are amino acid linkers. In some aspects, the first polypeptide has a structure represented by VL1-L1-VL2-L2-VH2-L3-VH1-L4-CH1-L5-Fc; VH1-L6-VH2-L7-VL2-L8-VL1-L9-CH1-L10-Fc; VL1-L11-VL2-L12-VH2-L13-VH1-L14-CL-L15-Fc; VH1-L16-VH2-L17-VL2-L18-VL1-L19-CL-L20-Fc; VL1-L21-VL2-L22-VH2-L23-VH1-L24-CH1-L25-CL-L26-Fc; VH1-L27-VH2-L28-VL2-L29-VL1-L30-CH1-L31-CL-L32-Fc; VL1-L33-VL2-L34-VH2-L35-VH1-L36-CL-L37-CH1-L38-Fc; or VH1-L39-VH2-L40-VL2-L41-VL1-L42-CL-L43-CH1-L44-Fc and the second polypeptide has a structure represented by VL3-L45-VL4-L46-VH4-L47-VH3-L48-CH1-L49-Fc. In some aspects, the first polypeptide has a structure represented by VL1-L1-VL2-L2-VH2-L3-VH1-L4-CH1-L5-Fc; VH1-L6-VH2-L7-VL2-L8-VL1-L9-CH1-L10-Fc; VL1-L11-VL2-L12-VH2-L13-VH1-L14-CL-L15-Fc; VH1-L16-VH2-L17-VL2-L18-VL1-L19-CL-L20-Fc; VL1-L21-VL2-L22-VH2-L23-VH1-L24-CH1-L25-CL-L26-Fc; VH1-L27-VH2-L28-VL2-L29-VL1-L30-CH1-L31-CL-L32-Fc; VL1-L33-VL2-L34-VH2-L35-VH1-L36-CL-L37-CH1-L38-Fc; or VH1-L39-VH2-L40-VL2-L41-VL1-L42-CL-L43-CH1-L44-Fc and the second polypeptide has a structure represented by VH3-L50-VH4-L51-VL4-L52-VL3-L53-CH1-L54-Fc. In some aspects, the first polypeptide has a structure represented by VL1-L1-VL2-L2-VH2-L3-VH1-L4-CH1-L5-Fc; VH1-L6-VH2-L7-VL2-L8-VL1-L9-CH1-L10-Fc; VL1-L11-VL2-L12-VH2-L13-VH1-L14-CL-L15-Fc; VH1-L16-VH2-L17-VL2-L18-VL1-L19-CL-L20-Fc; VL1-L21-VL2-L22-VH2-L23-VH1-L24-CH1-L25-CL-L26-Fc; VH1-L27-VH2-L28-VL2-L29-VL1-L30-CH1-L31-CL-L32-Fc; VL1-L33-VL2-L34-VH2-L35-VH1-L36-CL-L37-CH1-L38-Fc; or VH1-L39-VH2-L40-VL2-L41-VL1-L42-CL-L43-CH1-L44-Fc and the second polypeptide has a structure represented by VL3-L55-VL4-L56-VH4-L57-VH3-L58-CL-L59-Fc. In some aspects, the first polypeptide has a structure represented by VL1-L1-VL2-L2-VH2-L3-VH1-L4-CH1-L5-Fc; VH1-L6-VH2-L7-VL2-L8-VL1-L9-CH1-L10-Fc; VL1-L11-VL2-L12-VH2-L13-VH1-L14-CL-L15-Fc; VH1-L16-VH2-L17-VL2-L18-VL1-L19-CL-L20-Fc; VL1-L21-VL2-L22-VH2-L23-VH1-L24-CH1-L25-CL-L26-Fc; VH1-L27-VH2-L28-VL2-L29-VL1-L30-CH1-L31-CL-L32-Fc; VL1-L33-VL2-L34-VH2-L35-VH1-L36-CL-L37-CH1-L38-Fc; or VH1-L39-VH2-L40-VL2-L41-VL1-L42-CL-L43-CH1-L44-Fc and the second polypeptide has a structure represented by VH3-L60-VH4-L61-VL4-L62-VL3-L63-CL-L64-Fc. In some aspects, the first polypeptide has a structure represented by VL1-L1-VL2-L2-VH2-L3-VH1-L4-CH1-L5-Fc; VH1-L6-VH2-L7-VL2-L8-VL1-L9-CH1-L10-Fc; VL1-L11-VL2-L12-VH2-L13-VH1-L14-CL-L15-Fc; VH1-L16-VH2-L17-VL2-L18-VL1-L19-CL-L20-Fc; VL1-L21-VL2-L22-VH2-L23-VH1-L24-CH1-L25-CL-L26-Fc; VH1-L27-VH2-L28-VL2-L29-VL1-L30-CH1-L31-CL-L32-Fc; VL1-L33-VL2-L34-VH2-L35-VH1-L36-CL-L37-CH1-L38-Fc; or VH1-L39-VH2-L40-VL2-L41-VL1-L42-CL-L43-CH1-L44-Fc and the second polypeptide has a structure represented by VL3-L65-VL4-L66-VH4-L67-VH3-L68-CH1-L69-CL-L70-Fc. In some aspects, the first polypeptide has a structure represented by VL1-L1-VL2-L2-VH2-L3-VH1-L4-CH1-L5-Fc; VH1-L6-VH2-L7-VL2-L8-VL1-L9-CH1-L10-Fc; VL1-L11-VL2-L12-VH2-L13-VH1-L14-CL-L15-Fc; VH1-L16-VH2-L17-VL2-L18-VL1-L19-CL-L20-Fc; VL1-L21-VL2-L22-VH2-L23-VH1-L24-CH1-L25-CL-L26-Fc; VH1-L27-VH2-L28-VL2-L29-VL1-L30-CH1-L31-CL-L32-Fc; VL1-L33-VL2-L34-VH2-L35-VH1-L36-CL-L37-CH1-L38-Fc; or VH1-L39-VH2-L40-VL2-L41-VL1-L42-CL-L43-CH1-L44-Fc and the second polypeptide has a structure represented by VH3-L71-VH4-L72-VL4-L73-VL3-L74-CH1-L75-CL-L76-Fc. In some aspects, the first polypeptide has a structure represented by VL1-L1-VL2-L2-VH2-L3-VH1-L4-CH1-L5-Fc; VH1-L6-VH2-L7-VL2-L8-VL1-L9-CH1-L10-Fc; VL1-L11-VL2-L12-VH2-L13-VH1-L14-CL-L15-Fc; VH1-L16-VH2-L17-VL2-L18-VL1-L19-CL-L20-Fc; VL1-L21-VL2-L22-VH2-L23-VH1-L24-CH1-L25-CL-L26-Fc; VH1-L27-VH2-L28-VL2-L29-VL1-L30-CH1-L31-CL-L32-Fc; VL1-L33-VL2-L34-VH2-L35-VH1-L36-CL-L37-CH1-L38-Fc; or VH1-L39-VH2-L40-VL2-L41-VL1-L42-CL-L43-CH1-L44-Fc and the second polypeptide has a structure represented by VL3-L77-VL4-L78-VH4-L79-VH3-L80-CL-L81-CH1-L82-Fc. In some aspects, the first polypeptide has a structure represented by VL1-L1-VL2-L2-VH2-L3-VH1-L4-CH1-L5-Fc; VH1-L6-VH2-L7-VL2-L8-VL1-L9-CH1-L10-Fc; VL1-L11-VL2-L12-VH2-L13-VH1-L14-CL-L15-Fc; VH1-L16-VH2-L17-VL2-L18-VL1-L19-CL-L20-Fc; VL1-L21-VL2-L22-VH2-L23-VH1-L24-CH1-L25-CL-L26-Fc; VH1-L27-VH2-L28-VL2-L29-VL1-L30-CH1-L31-CL-L32-Fc; VL1-L33-VL2-L34-VH2-L35-VH1-L36-CL-L37-CH1-L38-Fc; or VH1-L39-VH2-L40-VL2-L41-VL1-L42-CL-L43-CH1-L44-Fc and the second polypeptide has a structure represented by VH3-L83-VH4-L84-VL4-L85-VL3-L86-CL-L87-CH1-L88-Fc. In some aspects, VL1 and VH1 may specifically bind to A2AR, APRIL, ATPDase, BAFF, BAFFR, BCMA, BlyS, BTK, BTLA, B7DC, B7H1, B7H2, B7H3, B7H4, B7H5, B7H6, B7H7, B7RP1, B7-4, C3, C5, CCL2, CCL3, CCL4, CCL5, CCL7, CCL8, CCL11, CCL15, CCL17, CCL19, CCL20, CCL21, CCL25, CCR3, CCR4, CD16A, CD19, CD20, CD24, CD27, CD28, CD30, CD38, CD39, CD40, CD40L, CD47, CD52, CD70, CD80, CD86, CD123, CD133, CD137, CD137L, CD160, CD272, CEACAM5, CLEC9, CLEC91, CRTH2, CSF-1, CSF-2, CSF-3, CXCL1, CXCL2, CXCL4, CXCL12, CXCL13, CXCR3, cMet, CTLA4, DLL3, DLL4, DNGR-1, E-cadherin, EGFR, ENTPD1, EpCAM, FCER1, FCER1A, FCER2, FGFR, FLAP, FOLH1, Gi24, GITR, GITRL, GPR5, GP100, GPRC5D, HER2, HER3, ICOSL, ICOS, HHLA2, HMGB1, HVEM, IDO, IFNa, IgE, IGF1R, IL2Rbeta, IL1, ILIA, IL1B, IL1F10, IL2, IL4, IL4Ra, IL5, IL5R, IL6, IL7, IL7Ra, IL8, IL9, IL9R, IL10, rhILlO, IL12, IL13, IL13Ra1, IL13Ra2, IL15, IL17, IL17Rb, IL18, IL22, IL23, IL25, IL7, IL33, IL35, ITGB4, ITK, KIR, LAG3, LAMP1, leptin, LPFS2, MHC class II, MUC-1, MUC-16, NCR3LG1, NKG2D, NKp46, NTPDase-1, OX40, OX40L, PD-1, PD-L1, PD-L2, PROM1, S152, SIRPalpha, SISP1, SLC, SPG64, ST2, STEAP1, STEAP2, Syk kinase, STEAP1, TROP2, TACI, TDO, TGFBETA, T14, TIGIT, TIM3, TLR, TLR2, TLR4, TLR5, TLR9, TMEF1, TNFa, TNFRSF7, Tp55, TREM1, TSLP, TSLPR, TWEAK, VEGF, VISTA, Vstm3, or WUCAM. In some aspects, VL4 and VH4 maythat specifically bind to A2AR, APRIL, ATPDase, BAFF, BAFFR, BCMA, BlyS, BTK, BTLA, B7DC, B7H1, B7H2, B7H3, B7H4, B7H5, B7H6, B7H7, B7RP1, B7-4, C3, C5, CCL2, CCL3, CCL4, CCL5, CCL7, CCL8, CCL11, CCL15, CCL17, CCL19, CCL20, CCL21, CCL25, CCR3, CCR4, CD16A, CD19, CD20, CD24, CD27, CD28, CD30, CD38, CD39, CD40, CD40L, CD47, CD52, CD70, CD80, CD86, CD123, CD133, CD137, CD137L, CD160, CD272, CEACAM5, CLEC9, CLEC91, CRTH2, CSF-1, CSF-2, CSF-3, CXCL1, CXCL2, CXCL4, CXCL12, CXCL13, CXCR3, cMet, CTLA4, DLL3, DLL4, DNGR-1, E-cadherin, EGFR, ENTPD1, EpCAM, FCER1, FCER1A, FCER2, FGFR, FLAP, FOLH1, Gi24, GITR, GITRL, GPR5, GP100, GPRC5D, HER2, HER3, ICOSL, ICOS, HHLA2, HMGB1, HVEM, IDO, IFNa, IgE, IGF1R, IL2Rbeta, IL1, ILIA, IL1B, IL1F10, IL2, IL4, IL4Ra, IL5, IL5R, IL6, IL7, IL7Ra, IL8, IL9, IL9R, IL10, rhILlO, IL12, IL13, IL13Ra1, IL13Ra2, IL15, IL17, IL17Rb, IL18, IL22, IL23, IL25, IL7, IL33, IL35, ITGB4, ITK, KIR, LAG3, LAMP1, leptin, LPFS2, MHC class II, MUC-1, MUC-16, NCR3LG1, NKG2D, NKp46, NTPDase-1, OX40, OX40L, PD-1, PD-L1, PD-L2, PROM1, S152, SIRPalpha, SISP1, SLC, SPG64, ST2, STEAP1, STEAP2, Syk kinase, STEAP1, TROP2, TACI, TDO, TGFBETA, T14, TIGIT, TIM3, TLR, TLR2, TLR4, TLR5, TLR9, TMEF1, TNFa, TNFRSF7, Tp55, TREM1, TSLP, TSLPR, TWEAK, VEGF, VISTA, Vstm3, or WUCAM. The VL1, VL4, VH1, and/or VH4 may specifically bind to cMet, Trop2, CD20, or CD19; VL2 and/or VH2 may specifically bind to CD28; and VL3 and/or VH3 may specifically bind to CD3. For example, the antigen binding polypeptide complex may specifically bind CD28, CD3, cMet, and Trop2. For example, the antigen binding polypeptide complex may specifically bind CD28, CD3, CD19, and CD20. For example, VL1 and VH1 may specifically bind to cMet. For example, VL4 and VH4 may specifically bind to cMet. For example, VL1 and VH1 may specifically bind to Trop2. For example, VL4 and VH4 may specifically bind to Trop2. For example, VL1 and VH1 may specifically bind to CD19. For example, VL4 and VH4 may specifically bind to CD19. For example, VL1 and VH1 may specifically bind to CD20. For example, VL4 and VH4 may specifically bind to CD20.

In some aspects, the invention is directed to an antigen binding polypeptide complex comprising a first polypeptide and a second polypeptide; wherein the first polypeptide has a structure represented by VL1-L1-VL2-L2-VH2-L3-VH1-L4-CH1-L5-Fc; VH1-L6-VH2-L7-VL2-L8-VL1-L9-CH1-L10-Fc; VL1-L11-VL2-L12-VH2-L13-VH1-L14-CL-L15-Fc; VH1-L16-VH2-L17-VL2-L18-VL1-L19-CL-L20-Fc; VL1-L21-VL2-L22-VH2-L23-VH1-L24-CH1-L25-CL-L26-Fc; VH1-L27-VH2-L28-VL2-L29-VL1-L30-CH1-L31-CL-L32-Fc; VL1-L33-VL2-L34-VH2-L35-VH1-L36-CL-L37-CH1-L38-Fc; or VH1-L39-VH2-L40-VL2-L41-VL1-L42-CL-L43-CH1-L44-Fc; wherein the second polypeptide has a structure represented by VL3-L45-VL4-L46-VH4-L47-VH3-L48-CH1-L49-Fc; VH3-L50-VH4-L51-VL4-L52-VL3-L53-CH1-L54-Fc; VL3-L55-VL4-L56-VH4-L57-VH3-L58-CL-L59-Fc; VH3-L60-VH4-L61-VL4-L62-VL3-L63-CL-L64-Fc; VL3-L65-VL4-L66-VH4-L67-VH3-L68-CH1-L69-CL-L70-Fc; VH3-L71-VH4-L72-VL4-L73-VL3-L74-CH1-L75-CL-L76-Fc; VL3-L77-VL4-L78-VH4-L79-VH3-L80-CL-L81-CH1-L82-Fc; or VH3-L83-VH4-L84-VL4-L85-VL3-L86-CL-L87-CH1-L88-Fc; wherein VL1 is a first immunoglobulin light chain variable region that specifically binds to A2AR, APRIL, ATPDase, BAFF, BAFFR, BCMA, BlyS, BTK, BTLA, B7DC, B7H1, B7H2, B7H3, B7H4, B7H5, B7H6, B7H7, B7RP1, B7-4, C3, C5, CCL2, CCL3, CCL4, CCL5, CCL7, CCL8, CCL11, CCL15, CCL17, CCL19, CCL20, CCL21, CCL25, CCR3, CCR4, CD16A, CD19, CD20, CD24, CD27, CD28, CD30, CD38, CD39, CD40, CD40L, CD47, CD52, CD70, CD80, CD86, CD123, CD133, CD137, CD137L, CD160, CD272, CEACAM5, CLEC9, CLEC91, CRTH2, CSF-1, CSF-2, CSF-3, CXCL1, CXCL2, CXCL4, CXCL12, CXCL13, CXCR3, cMet, CTLA4, DLL3, DLL4, DNGR-1, E-cadherin, EGFR, ENTPD1, EpCAM, FCER1, FCER1A, FCER2, FGFR, FLAP, FOLH1, Gi24, GITR, GITRL, GPR5, GP100, GPRC5D, HER2, HER3, ICOSL, ICOS, HHLA2, HMGB1, HVEM, IDO, IFNa, IgE, IGF1R, IL2Rbeta, IL1, ILIA, IL1B, IL1F10, IL2, IL4, IL4Ra, IL5, IL5R, IL6, IL7, IL7Ra, IL8, IL9, IL9R, IL10, rhILlO, IL12, IL13, IL13Ra1, IL13Ra2, IL15, IL17, IL17Rb, IL18, IL22, IL23, IL25, IL7, IL33, IL35, ITGB4, ITK, KIR, LAG3, LAMP1, leptin, LPFS2, MHC class II, MUC-1, MUC-16, NCR3LG1, NKG2D, NKp46, NTPDase-1, OX40, OX40L, PD-1, PD-L1, PD-L2, PROM1, 5152, SIRPalpha, SISP1, SLC, SPG64, ST2, STEAP1, STEAP2, Syk kinase, STEAP1, TROP2, TACI, TDO, TGFBETA, T14, TIGIT, TIM3, TLR, TLR2, TLR4, TLR5, TLR9, TMEF1, TNFa, TNFRSF7, Tp55, TREM1, TSLP, TSLPR, TWEAK, VEGF, VISTA, Vstm3, or WUCAM; VL2 is a second immunoglobulin light chain variable region that specifically binds to A2AR, APRIL, ATPDase, BAFF, BAFFR, BCMA, BlyS, BTK, BTLA, B7DC, B7H1, B7H2, B7H3, B7H4, B7H5, B7H6, B7H7, B7RP1, B7-4, C3, C5, CCL2, CCL3, CCL4, CCL5, CCL7, CCL8, CCL11, CCL15, CCL17, CCL19, CCL20, CCL21, CCL25, CCR3, CCR4, CD16A, CD19, CD20, CD24, CD27, CD28, CD30, CD38, CD39, CD40, CD40L, CD47, CD52, CD70, CD80, CD86, CD123, CD133, CD137, CD137L, CD160, CD272, CEACAM5, CLEC9, CLEC91, CRTH2, CSF-1, CSF-2, CSF-3, CXCL1, CXCL2, CXCL4, CXCL12, CXCL13, CXCR3, cMet, CTLA4, DLL3, DLL4, DNGR-1, E-cadherin, EGFR, ENTPD1, EpCAM, FCER1, FCER1A, FCER2, FGFR, FLAP, FOLH1, Gi24, GITR, GITRL, GPR5, GP100, GPRC5D, HER2, HER3, ICOSL, ICOS, HHLA2, HMGB1, HVEM, IDO, IFNa, IgE, IGF1R, IL2Rbeta, IL1, ILIA, IL1B, IL1F10, IL2, IL4, IL4Ra, IL5, IL5R, IL6, IL7, IL7Ra, IL8, IL9, IL9R, IL10, rhILlO, IL12, IL13, IL13Ra1, IL13Ra2, IL15, IL17, IL17Rb, IL18, IL22, IL23, IL25, IL7, IL33, IL35, ITGB4, ITK, KIR, LAG3, LAMP1, leptin, LPFS2, MHC class II, MUC-1, MUC-16, NCR3LG1, NKG2D, NKp46, NTPDase-1, OX40, OX40L, PD-1, PD-L1, PD-L2, PROM1, 5152, SIRPalpha, SISP1, SLC, SPG64, ST2, STEAP1, STEAP2, Syk kinase, STEAP1, TROP2, TACI, TDO, TGFBETA, T14, TIGIT, TIM3, TLR, TLR2, TLR4, TLR5, TLR9, TMEF1, TNFa, TNFRSF7, Tp55, TREM1, TSLP, TSLPR, TWEAK, VEGF, VISTA, Vstm3, or WUCAM; VL3 is a third immunoglobulin light chain variable region that specifically binds to CD3; VL4 is a fourth immunoglobulin light chain variable region that specifically binds to CD28; VH1 is a first immunoglobulin heavy chain variable region that specifically binds to A2AR, APRIL, ATPDase, BAFF, BAFFR, BCMA, BlyS, BTK, BTLA, B7DC, B7H1, B7H2, B7H3, B7H4, B7H5, B7H6, B7H7, B7RP1, B7-4, C3, C5, CCL2, CCL3, CCL4, CCL5, CCL7, CCL8, CCL11, CCL15, CCL17, CCL19, CCL20, CCL21, CCL25, CCR3, CCR4, CD16A, CD19, CD20, CD24, CD27, CD28, CD30, CD38, CD39, CD40, CD40L, CD47, CD52, CD70, CD80, CD86, CD123, CD133, CD137, CD137L, CD160, CD272, CEACAM5, CLEC9, CLEC91, CRTH2, CSF-1, CSF-2, CSF-3, CXCL1, CXCL2, CXCL4, CXCL12, CXCL13, CXCR3, cMet, CTLA4, DLL3, DLL4, DNGR-1, E-cadherin, EGFR, ENTPD1, EpCAM, FCER1, FCER1A, FCER2, FGFR, FLAP, FOLH1, Gi24, GITR, GITRL, GPR5, GP100, GPRC5D, HER2, HER3, ICOSL, ICOS, HHLA2, HMGB1, HVEM, IDO, IFNa, IgE, IGF1R, IL2Rbeta, IL1, ILIA, IL1B, IL1F10, IL2, IL4, IL4Ra, IL5, IL5R, IL6, IL7, IL7Ra, IL8, IL9, IL9R, IL10, rhILlO, IL12, IL13, IL13Ra1, IL13Ra2, IL15, IL17, IL17Rb, IL18, IL22, IL23, IL25, IL7, IL33, IL35, ITGB4, ITK, KIR, LAG3, LAMP1, leptin, LPFS2, MHC class II, MUC-1, MUC-16, NCR3LG1, NKG2D, NKp46, NTPDase-1, OX40, OX40L, PD-1, PD-L1, PD-L2, PROM1, S152, SIRPalpha, SISP1, SLC, SPG64, ST2, STEAP1, STEAP2, Syk kinase, STEAP1, TROP2, TACI, TDO, TGFBETA, T14, TIGIT, TIM3, TLR, TLR2, TLR4, TLR5, TLR9, TMEF1, TNFa, TNFRSF7, Tp55, TREM1, TSLP, TSLPR, TWEAK, VEGF, VISTA, Vstm3, or WUCAM; VH2 is a second immunoglobulin heavy chain variable region that specifically binds to A2AR, APRIL, ATPDase, BAFF, BAFFR, BCMA, BlyS, BTK, BTLA, B7DC, B7H1, B7H2, B7H3, B7H4, B7H5, B7H6, B7H7, B7RP1, B7-4, C3, C5, CCL2, CCL3, CCL4, CCL5, CCL7, CCL8, CCL11, CCL15, CCL17, CCL19, CCL20, CCL21, CCL25, CCR3, CCR4, CD16A, CD19, CD20, CD24, CD27, CD28, CD30, CD38, CD39, CD40, CD40L, CD47, CD52, CD70, CD80, CD86, CD123, CD133, CD137, CD137L, CD160, CD272, CEACAM5, CLEC9, CLEC91, CRTH2, CSF-1, CSF-2, CSF-3, CXCL1, CXCL2, CXCL4, CXCL12, CXCL13, CXCR3, cMet, CTLA4, DLL3, DLL4, DNGR-1, E-cadherin, EGFR, ENTPD1, EpCAM, FCER1, FCER1A, FCER2, FGFR, FLAP, FOLH1, Gi24, GITR, GITRL, GPR5, GP100, GPRC5D, HER2, HER3, ICOSL, ICOS, HHLA2, HMGB1, HVEM, IDO, IFNa, IgE, IGF1R, IL2Rbeta, IL1, ILIA, IL1B, IL1F10, IL2, IL4, IL4Ra, IL5, IL5R, IL6, IL7, IL7Ra, IL8, IL9, IL9R, IL10, rhILlO, IL12, IL13, IL13Ra1, IL13Ra2, IL15, IL17, IL17Rb, IL18, IL22, IL23, IL25, IL7, IL33, IL35, ITGB4, ITK, KIR, LAG3, LAMP1, leptin, LPFS2, MHC class II, MUC-1, MUC-16, NCR3LG1, NKG2D, NKp46, NTPDase-1, OX40, OX40L, PD-1, PD-L1, PD-L2, PROM1, S152, SIRPalpha, SISP1, SLC, SPG64, ST2, STEAP1, STEAP2, Syk kinase, STEAP1, TROP2, TACI, TDO, TGFBETA, T14, TIGIT, TIM3, TLR, TLR2, TLR4, TLR5, TLR9, TMEF1, TNFa, TNFRSF7, Tp55, TREM1, TSLP, TSLPR, TWEAK, VEGF, VISTA, Vstm3, or WUCAM; VH3 is a third immunoglobulin heavy chain variable region that specifically binds to CD3; VH4 is a fourth immunoglobulin heavy chain variable region that specifically binds to CD28; Fc is a region comprising an immunoglobulin heavy chain constant region 2 (CH2), an immunoglobulin heavy chain constant region 3 (CH3), and optionally, an immunoglobulin hinge; CH1 is an immunoglobulin heavy chain constant region 1; CL is an immunoglobulin light chain constant region; and L1-L88 are amino acid linkers. In some aspects, the first polypeptide has a structure represented by VL1-L1-VL2-L2-VH2-L3-VH1-L4-CH1-L5-Fc; VH1-L6-VH2-L7-VL2-L8-VL1-L9-CH1-L10-Fc; VL1-L11-VL2-L12-VH2-L13-VH1-L14-CL-L15-Fc; VH1-L16-VH2-L17-VL2-L18-VL1-L19-CL-L20-Fc; VL1-L21-VL2-L22-VH2-L23-VH1-L24-CH1-L25-CL-L26-Fc; VH1-L27-VH2-L28-VL2-L29-VL1-L30-CH1-L31-CL-L32-Fc; VL1-L33-VL2-L34-VH2-L35-VH1-L36-CL-L37-CH1-L38-Fc; or VH1-L39-VH2-L40-VL2-L41-VL1-L42-CL-L43-CH1-L44-Fc and the second polypeptide has a structure represented by VL3-L45-VL4-L46-VH4-L47-VH3-L48-CH1-L49-Fc. In some aspects, the first polypeptide has a structure represented by VL1-L1-VL2-L2-VH2-L3-VH1-L4-CH1-L5-Fc; VH1-L6-VH2-L7-VL2-L8-VL1-L9-CH1-L10-Fc; VL1-L11-VL2-L12-VH2-L13-VH1-L14-CL-L15-Fc; VH1-L16-VH2-L17-VL2-L18-VL1-L19-CL-L20-Fc; VL1-L21-VL2-L22-VH2-L23-VH1-L24-CH1-L25-CL-L26-Fc; VH1-L27-VH2-L28-VL2-L29-VL1-L30-CH1-L31-CL-L32-Fc; VL1-L33-VL2-L34-VH2-L35-VH1-L36-CL-L37-CH1-L38-Fc; or VH1-L39-VH2-L40-VL2-L41-VL1-L42-CL-L43-CH1-L44-Fc and the second polypeptide has a structure represented by VH3-L50-VH4-L51-VL4-L52-VL3-L53-CH1-L54-Fc. In some aspects, the first polypeptide has a structure represented by VL1-L1-VL2-L2-VH2-L3-VH1-L4-CH1-L5-Fc; VH1-L6-VH2-L7-VL2-L8-VL1-L9-CH1-L10-Fc; VL1-L11-VL2-L12-VH2-L13-VH1-L14-CL-L15-Fc; VH1-L16-VH2-L17-VL2-L18-VL1-L19-CL-L20-Fc; VL1-L21-VL2-L22-VH2-L23-VH1-L24-CH1-L25-CL-L26-Fc; VH1-L27-VH2-L28-VL2-L29-VL1-L30-CH1-L31-CL-L32-Fc; VL1-L33-VL2-L34-VH2-L35-VH1-L36-CL-L37-CH1-L38-Fc; or VH1-L39-VH2-L40-VL2-L41-VL1-L42-CL-L43-CH1-L44-Fc and the second polypeptide has a structure represented by VL3-L55-VL4-L56-VH4-L57-VH3-L58-CL-L59-Fc. In some aspects, the first polypeptide has a structure represented by VL1-L1-VL2-L2-VH2-L3-VH1-L4-CH1-L5-Fc; VH1-L6-VH2-L7-VL2-L8-VL1-L9-CH1-L10-Fc; VL1-L11-VL2-L12-VH2-L13-VH1-L14-CL-L15-Fc; VH1-L16-VH2-L17-VL2-L18-VL1-L19-CL-L20-Fc; VL1-L21-VL2-L22-VH2-L23-VH1-L24-CH1-L25-CL-L26-Fc; VH1-L27-VH2-L28-VL2-L29-VL1-L30-CH1-L31-CL-L32-Fc; VL1-L33-VL2-L34-VH2-L35-VH1-L36-CL-L37-CH1-L38-Fc; or VH1-L39-VH2-L40-VL2-L41-VL1-L42-CL-L43-CH1-L44-Fc and the second polypeptide has a structure represented by VH3-L60-VH4-L61-VL4-L62-VL3-L63-CL-L64-Fc. In some aspects, the first polypeptide has a structure represented by VL1-L1-VL2-L2-VH2-L3-VH1-L4-CH1-L5-Fc; VH1-L6-VH2-L7-VL2-L8-VL1-L9-CH1-L10-Fc; VL1-L11-VL2-L12-VH2-L13-VH1-L14-CL-L15-Fc; VH1-L16-VH2-L17-VL2-L18-VL1-L19-CL-L20-Fc; VL1-L21-VL2-L22-VH2-L23-VH1-L24-CH1-L25-CL-L26-Fc; VH1-L27-VH2-L28-VL2-L29-VL1-L30-CH1-L31-CL-L32-Fc; VL1-L33-VL2-L34-VH2-L35-VH1-L36-CL-L37-CH1-L38-Fc; or VH1-L39-VH2-L40-VL2-L41-VL1-L42-CL-L43-CH1-L44-Fc and the second polypeptide has a structure represented by VL3-L65-VL4-L66-VH4-L67-VH3-L68-CH1-L69-CL-L70-Fc. In some aspects, the first polypeptide has a structure represented by VL1-L1-VL2-L2-VH2-L3-VH1-L4-CH1-L5-Fc; VH1-L6-VH2-L7-VL2-L8-VL1-L9-CH1-L10-Fc; VL1-L11-VL2-L12-VH2-L13-VH1-L14-CL-L15-Fc; VH1-L16-VH2-L17-VL2-L18-VL1-L19-CL-L20-Fc; VL1-L21-VL2-L22-VH2-L23-VH1-L24-CH1-L25-CL-L26-Fc; VH1-L27-VH2-L28-VL2-L29-VL1-L30-CH1-L31-CL-L32-Fc; VL1-L33-VL2-L34-VH2-L35-VH1-L36-CL-L37-CH1-L38-Fc; or VH1-L39-VH2-L40-VL2-L41-VL1-L42-CL-L43-CH1-L44-Fc and the second polypeptide has a structure represented by VH3-L71-VH4-L72-VL4-L73-VL3-L74-CH1-L75-CL-L76-Fc. In some aspects, the first polypeptide has a structure represented by VL1-L1-VL2-L2-VH2-L3-VH1-L4-CH1-L5-Fc; VH1-L6-VH2-L7-VL2-L8-VL1-L9-CH1-L10-Fc; VL1-L11-VL2-L12-VH2-L13-VH1-L14-CL-L15-Fc; VH1-L16-VH2-L17-VL2-L18-VL1-L19-CL-L20-Fc; VL1-L21-VL2-L22-VH2-L23-VH1-L24-CH1-L25-CL-L26-Fc; VH1-L27-VH2-L28-VL2-L29-VL1-L30-CH1-L31-CL-L32-Fc; VL1-L33-VL2-L34-VH2-L35-VH1-L36-CL-L37-CH1-L38-Fc; or VH1-L39-VH2-L40-VL2-L41-VL1-L42-CL-L43-CH1-L44-Fc and the second polypeptide has a structure represented by VL3-L77-VL4-L78-VH4-L79-VH3-L80-CL-L81-CH1-L82-Fc. In some aspects, the first polypeptide has a structure represented by VL1-L1-VL2-L2-VH2-L3-VH1-L4-CH1-L5-Fc; VH1-L6-VH2-L7-VL2-L8-VL1-L9-CH1-L10-Fc; VL1-L11-VL2-L12-VH2-L13-VH1-L14-CL-L15-Fc; VH1-L16-VH2-L17-VL2-L18-VL1-L19-CL-L20-Fc; VL1-L21-VL2-L22-VH2-L23-VH1-L24-CH1-L25-CL-L26-Fc; VH1-L27-VH2-L28-VL2-L29-VL1-L30-CH1-L31-CL-L32-Fc; VL1-L33-VL2-L34-VH2-L35-VH1-L36-CL-L37-CH1-L38-Fc; or VH1-L39-VH2-L40-VL2-L41-VL1-L42-CL-L43-CH1-L44-Fc and the second polypeptide has a structure represented by VH3-L83-VH4-L84-VL4-L85-VL3-L86-CL-L87-CH1-L88-Fc. In some aspects, VL1 and VH1 may specifically bind to A2AR, APRIL, ATPDase, BAFF, BAFFR, BCMA, BlyS, BTK, BTLA, B7DC, B7H1, B7H2, B7H3, B7H4, B7H5, B7H6, B7H7, B7RP1, B7-4, C3, C5, CCL2, CCL3, CCL4, CCL5, CCL7, CCL8, CCL11, CCL15, CCL17, CCL19, CCL20, CCL21, CCL25, CCR3, CCR4, CD16A, CD19, CD20, CD24, CD27, CD28, CD30, CD38, CD39, CD40, CD40L, CD47, CD52, CD70, CD80, CD86, CD123, CD133, CD137, CD137L, CD160, CD272, CEACAM5, CLEC9, CLEC91, CRTH2, CSF-1, CSF-2, CSF-3, CXCL1, CXCL2, CXCL4, CXCL12, CXCL13, CXCR3, cMet, CTLA4, DLL3, DLL4, DNGR-1, E-cadherin, EGFR, ENTPD1, EpCAM, FCER1, FCER1A, FCER2, FGFR, FLAP, FOLH1, Gi24, GITR, GITRL, GPR5, GP100, GPRC5D, HER2, HER3, ICOSL, ICOS, HHLA2, HMGB1, HVEM, IDO, IFNa, IgE, IGF1R, IL2Rbeta, IL1, ILIA, IL1B, IL1F10, IL2, IL4, IL4Ra, IL5, IL5R, IL6, IL7, IL7Ra, IL8, IL9, IL9R, IL10, rhILlO, IL12, IL13, IL13Ra1, IL13Ra2, IL15, IL17, IL17Rb, IL18, IL22, IL23, IL25, IL7, IL33, IL35, ITGB4, ITK, KIR, LAG3, LAMP1, leptin, LPFS2, MHC class II, MUC-1, MUC-16, NCR3LG1, NKG2D, NKp46, NTPDase-1, OX40, OX40L, PD-1, PD-L1, PD-L2, PROM1, 5152, SIRPalpha, SISP1, SLC, SPG64, ST2, STEAP1, STEAP2, Syk kinase, STEAP1, TROP2, TACI, TDO, TGFBETA, T14, TIGIT, TIM3, TLR, TLR2, TLR4, TLR5, TLR9, TMEF1, TNFa, TNFRSF7, Tp55, TREM1, TSLP, TSLPR, TWEAK, VEGF, VISTA, Vstm3, or WUCAM. In some aspects, VL2 and VH2 may specifically bind to A2AR, APRIL, ATPDase, BAFF, BAFFR, BCMA, BlyS, BTK, BTLA, B7DC, B7H1, B7H2, B7H3, B7H4, B7H5, B7H6, B7H7, B7RP1, B7-4, C3, C5, CCL2, CCL3, CCL4, CCL5, CCL7, CCL8, CCL11, CCL15, CCL17, CCL19, CCL20, CCL21, CCL25, CCR3, CCR4, CD16A, CD19, CD20, CD24, CD27, CD28, CD30, CD38, CD39, CD40, CD40L, CD47, CD52, CD70, CD80, CD86, CD123, CD133, CD137, CD137L, CD160, CD272, CEACAM5, CLEC9, CLEC91, CRTH2, CSF-1, CSF-2, CSF-3, CXCL1, CXCL2, CXCL4, CXCL12, CXCL13, CXCR3, cMet, CTLA4, DLL3, DLL4, DNGR-1, E-cadherin, EGFR, ENTPD1, EpCAM, FCER1, FCER1A, FCER2, FGFR, FLAP, FOLH1, Gi24, GITR, GITRL, GPR5, GP100, GPRC5D, HER2, HER3, ICOSL, ICOS, HHLA2, HMGB1, HVEM, IDO, IFNa, IgE, IGF1R, IL2Rbeta, IL1, ILIA, IL1B, IL1F10, IL2, IL4, IL4Ra, IL5, IL5R, IL6, IL7, IL7Ra, IL8, IL9, IL9R, IL10, rhILlO, IL12, IL13, IL13Ra1, IL13Ra2, IL15, IL17, IL17Rb, IL18, IL22, IL23, IL25, IL7, IL33, IL35, ITGB4, ITK, KIR, LAG3, LAMP1, leptin, LPFS2, MHC class II, MUC-1, MUC-16, NCR3LG1, NKG2D, NKp46, NTPDase-1, OX40, OX40L, PD-1, PD-L1, PD-L2, PROM1, S152, SIRPalpha, SISP1, SLC, SPG64, ST2, STEAP1, STEAP2, Syk kinase, STEAP1, TROP2, TACI, TDO, TGFBETA, T14, TIGIT, TIM3, TLR, TLR2, TLR4, TLR5, TLR9, TMEF1, TNFa, TNFRSF7, Tp55, TREM1, TSLP, TSLPR, TWEAK, VEGF, VISTA, Vstm3, or WUCAM. The VL1, VL2, VH1, and/or VH2 may specifically bind to cMet, Trop2, CD20, or CD19; VL3 and/or VH3 may specifically bind to CD3; and VL4 and/or VH4 may specifically bind to CD28. For example, the antigen binding polypeptide complex may specifically bind CD28, CD3, cMet, and Trop2. For example, the antigen binding polypeptide complex may specifically bind CD28, CD3, CD19, and CD20. For example, VL1 and VH1 may specifically bind to cMet. For example, VL2 and VH2 may specifically bind to cMet. For example, VL1 and VH1 may specifically bind to Trop2. For example, VL2 and VH2 may specifically bind to Trop2. For example, VL1 and VH1 may specifically bind to CD19. For example, VL2 and VH2 may specifically bind to CD19. For example, VL1 and VH1 may specifically bind to CD20. For example, VL2 and VH2 may specifically bind to CD20.

In some aspects of the antibody polypeptide complex described herein, VL3 and VH3 specifically bind to CD3, VL4 and VH4 specifically bind to CD28, VL2 and VH2 specifically bind to Trop2, and VL1 and VH1 specifically bind to cMet. In some aspects, VL3 and VH3 specifically bind to CD3, VL4 and VH4 specifically bind to CD28, VL2 and VH2 specifically bind to cMet, and VL1 and VH1 specifically bind to Trop2. In some aspects, VL3 and VH3 specifically bind to CD3, VL4 and VH4 specifically bind to Trop2, VL2 and VH2 specifically bind to CD28, and VL1 and VH1 specifically bind to cMet. In some aspects, VL3 and VH3 specifically bind to CD3, VL4 and VH4 specifically bind to Trop2, VL2 and VH2 specifically bind to cMet, and VL1 and VH1 specifically bind to CD28. In some aspects, VL3 and VH3 specifically bind to CD3, VL4 and VH4 specifically bind to cMet, VL2 and VH2 specifically bind to Trop2, and VL1 and VH1 specifically bind to CD28. In some aspects, VL3 and VH3 specifically bind to CD3, VL4 and VH4 specifically bind to cMet, VL2 and VH2 specifically bind to CD28, and VL1 and VH1 specifically bind to Trop2. In some aspects, VL3 and VH3 specifically bind to CD3, VL4 and VH4 specifically bind to CD28, VL2 and VH2 specifically bind to CD20, and VL1 and VH1 specifically bind to CD19. In some aspects, VL3 and VH3 specifically bind to CD3, VL4 and VH4 specifically bind to CD28, VL2 and VH2 specifically bind to CD19, and VL1 and VH1 specifically bind to CD20. In some aspects, VL3 and VH3 specifically bind to CD3, VL4 and VH4 specifically bind to CD20, VL2 and VH2 specifically bind to CD28, and VL1 and VH1 specifically bind to CD19. In some aspects, VL3 and VH3 specifically bind to CD3, VL4 and VH4 specifically bind to CD20, VL2 and VH2 specifically bind to CD19, and VL1 and VH1 specifically bind to CD28. In some aspects, VL3 and VH3 specifically bind to CD3, VL4 and VH4 specifically bind to CD19, VL2 and VH2 specifically bind to CD20, and VL1 and VH1 specifically bind to CD28. In some aspects, VL3 and VH3 specifically bind to CD3, VL4 and VH4 specifically bind to CD19, VL2 and VH2 specifically bind to CD28, and VL1 and VH1 specifically bind to CD20.

In some aspects, the invention is directed to an antigen binding polypeptide complex comprising a first polypeptide and a second polypeptide; wherein the first polypeptide has a structure represented by VL1-L1-VL2-L2-VH2-L3-VH1-L4-CH1-L5-Fc; VH1-L6-VH2-L7-VL2-L8-VL1-L9-CH1-L10-Fc; VL1-L11-VL2-L12-VH2-L13-VH1-L14-CL-L15-Fc; VH1-L16-VH2-L17-VL2-L18-VL1-L19-CL-L20-Fc; VL1-L21-VL2-L22-VH2-L23-VH1-L24-CH1-L25-CL-L26-Fc; VH1-L27-VH2-L28-VL2-L29-VL1-L30-CH1-L31-CL-L32-Fc; VL1-L33-VL2-L34-VH2-L35-VH1-L36-CL-L37-CH1-L38-Fc; or VH1-L39-VH2-L40-VL2-L41-VL1-L42-CL-L43-CH1-L44-Fc; wherein the second polypeptide has a structure represented by VL3-L45-VL4-L46-VH4-L47-VH3-L48-CH1-L49-Fc; VH3-L50-VH4-L51-VL4-L52-VL3-L53-CH1-L54-Fc; VL3-L55-VL4-L56-VH4-L57-VH3-L58-CL-L59-Fc; VH3-L60-VH4-L61-VL4-L62-VL3-L63-CL-L64-Fc; VL3-L65-VL4-L66-VH4-L67-VH3-L68-CH1-L69-CL-L70-Fc; VH3-L71-VH4-L72-VL4-L73-VL3-L74-CH1-L75-CL-L76-Fc; VL3-L77-VL4-L78-VH4-L79-VH3-L80-CL-L81-CH1-L82-Fc; or VH3-L83-VH4-L84-VL4-L85-VL3-L86-CL-L87-CH1-L88-Fc; wherein VL1 is a first immunoglobulin light chain variable region that specifically binds to A2AR, APRIL, ATPDase, BAFF, BAFFR, BCMA, BlyS, BTK, BTLA, B7DC, B7H1, B7H2, B7H3, B7H4, B7H5, B7H6, B7H7, B7RP1, B7-4, C3, C5, CCL2, CCL3, CCL4, CCL5, CCL7, CCL8, CCL11, CCL15, CCL17, CCL19, CCL20, CCL21, CCL25, CCR3, CCR4, CD16A, CD19, CD20, CD24, CD27, CD28, CD30, CD38, CD39, CD40, CD40L, CD47, CD52, CD70, CD80, CD86, CD123, CD133, CD137, CD137L, CD160, CD272, CEACAM5, CLEC9, CLEC91, CRTH2, CSF-1, CSF-2, CSF-3, CXCL1, CXCL2, CXCL4, CXCL12, CXCL13, CXCR3, cMet, CTLA4, DLL3, DLL4, DNGR-1, E-cadherin, EGFR, ENTPD1, EpCAM, FCER1, FCER1A, FCER2, FGFR, FLAP, FOLH1, Gi24, GITR, GITRL, GPR5, GP100, GPRC5D, HER2, HER3, ICOSL, ICOS, HHLA2, HMGB1, HVEM, IDO, IFNa, IgE, IGF1R, IL2Rbeta, IL1, ILIA, IL1B, IL1F10, IL2, IL4, IL4Ra, IL5, IL5R, IL6, IL7, IL7Ra, IL8, IL9, IL9R, IL10, rhILlO, IL12, IL13, IL13Ra1, IL13Ra2, IL15, IL17, IL17Rb, IL18, IL22, IL23, IL25, IL7, IL33, IL35, ITGB4, ITK, KIR, LAG3, LAMP1, leptin, LPFS2, MHC class II, MUC-1, MUC-16, NCR3LG1, NKG2D, NKp46, NTPDase-1, OX40, OX40L, PD-1, PD-L1, PD-L2, PROM1, S152, SIRPalpha, SISP1, SLC, SPG64, ST2, STEAP1, STEAP2, Syk kinase, STEAP1, TROP2, TACI, TDO, TGFBETA, T14, TIGIT, TIM3, TLR, TLR2, TLR4, TLR5, TLR9, TMEF1, TNFa, TNFRSF7, Tp55, TREM1, TSLP, TSLPR, TWEAK, VEGF, VISTA, Vstm3, or WUCAM; VL2 is a second immunoglobulin light chain variable region that specifically binds to A2AR, APRIL, ATPDase, BAFF, BAFFR, BCMA, BlyS, BTK, BTLA, B7DC, B7H1, B7H2, B7H3, B7H4, B7H5, B7H6, B7H7, B7RP1, B7-4, C3, C5, CCL2, CCL3, CCL4, CCL5, CCL7, CCL8, CCL11, CCL15, CCL17, CCL19, CCL20, CCL21, CCL25, CCR3, CCR4, CD16A, CD19, CD20, CD24, CD27, CD28, CD30, CD38, CD39, CD40, CD40L, CD47, CD52, CD70, CD80, CD86, CD123, CD133, CD137, CD137L, CD160, CD272, CEACAM5, CLEC9, CLEC91, CRTH2, CSF-1, CSF-2, CSF-3, CXCL1, CXCL2, CXCL4, CXCL12, CXCL13, CXCR3, cMet, CTLA4, DLL3, DLL4, DNGR-1, E-cadherin, EGFR, ENTPD1, EpCAM, FCER1, FCER1A, FCER2, FGFR, FLAP, FOLH1, Gi24, GITR, GITRL, GPR5, GP100, GPRC5D, HER2, HER3, ICOSL, ICOS, HHLA2, HMGB1, HVEM, IDO, IFNa, IgE, IGF1R, IL2Rbeta, IL1, ILIA, IL1B, IL1F10, IL2, IL4, IL4Ra, IL5, IL5R, IL6, IL7, IL7Ra, IL8, IL9, IL9R, IL10, rhILlO, IL12, IL13, IL13Ra1, IL13Ra2, IL15, IL17, IL17Rb, IL18, IL22, IL23, IL25, IL7, IL33, IL35, ITGB4, ITK, KIR, LAG3, LAMP1, leptin, LPFS2, MHC class II, MUC-1, MUC-16, NCR3LG1, NKG2D, NKp46, NTPDase-1, OX40, OX40L, PD-1, PD-L1, PD-L2, PROM1, S152, SIRPalpha, SISP1, SLC, SPG64, ST2, STEAP1, STEAP2, Syk kinase, STEAP1, TROP2, TACI, TDO, TGFBETA, T14, TIGIT, TIM3, TLR, TLR2, TLR4, TLR5, TLR9, TMEF1, TNFa, TNFRSF7, Tp55, TREM1, TSLP, TSLPR, TWEAK, VEGF, VISTA, Vstm3, or WUCAM; VL3 is a third immunoglobulin light chain variable region that specifically binds to A2AR, APRIL, ATPDase, BAFF, BAFFR, BCMA, BlyS, BTK, BTLA, B7DC, B7H1, B7H2, B7H3, B7H4, B7H5, B7H6, B7H7, B7RP1, B7-4, C3, C5, CCL2, CCL3, CCL4, CCL5, CCL7, CCL8, CCL11, CCL15, CCL17, CCL19, CCL20, CCL21, CCL25, CCR3, CCR4, CD16A, CD19, CD20, CD24, CD27, CD28, CD30, CD38, CD39, CD40, CD40L, CD47, CD52, CD70, CD80, CD86, CD123, CD133, CD137, CD137L, CD160, CD272, CEACAM5, CLEC9, CLEC91, CRTH2, CSF-1, CSF-2, CSF-3, CXCL1, CXCL2, CXCL4, CXCL12, CXCL13, CXCR3, cMet, CTLA4, DLL3, DLL4, DNGR-1, E-cadherin, EGFR, ENTPD1, EpCAM, FCER1, FCER1A, FCER2, FGFR, FLAP, FOLH1, Gi24, GITR, GITRL, GPR5, GP100, GPRC5D, HER2, HER3, ICOSL, ICOS, HHLA2, HMGB1, HVEM, IDO, IFNa, IgE, IGF1R, IL2Rbeta, IL1, ILIA, IL1B, IL1F10, IL2, IL4, IL4Ra, IL5, TL5R, IL6, IL7, IL7Ra, IL8, IL9, IL9R, IL10, rhILlO, IL12, IL13, IL13Ra1, IL13Ra2, IL15, IL17, IL17Rb, IL18, IL22, IL23, IL25, IL7, IL33, IL35, ITGB4, ITK, KIR, LAG3, LAMP1, leptin, LPFS2, MHC class II, MUC-1, MUC-16, NCR3LG1, NKG2D, NKp46, NTPDase-1, OX40, OX40L, PD-1, PD-L1, PD-L2, PROM1, S152, SIRPalpha, SISP1, SLC, SPG64, ST2, STEAP1, STEAP2, Syk kinase, STEAP1, TROP2, TACI, TDO, TGFBETA, T14, TIGIT, TIM3, TLR, TLR2, TLR4, TLR5, TLR9, TMEF1, TNFa, TNFRSF7, Tp55, TREM1, TSLP, TSLPR, TWEAK, VEGF, VISTA, Vstm3, or WUCAM; VL4 is a fourth immunoglobulin light chain variable region that specifically binds to CD3; VH1 is a first immunoglobulin heavy chain variable region that specifically binds to A2AR, APRIL, ATPDase, BAFF, BAFFR, BCMA, BlyS, BTK, BTLA, B7DC, B7H1, B7H2, B7H3, B7H4, B7H5, B7H6, B7H7, B7RP1, B7-4, C3, C5, CCL2, CCL3, CCL4, CCL5, CCL7, CCL8, CCL11, CCL15, CCL17, CCL19, CCL20, CCL21, CCL25, CCR3, CCR4, CD16A, CD19, CD20, CD24, CD27, CD28, CD30, CD38, CD39, CD40, CD40L, CD47, CD52, CD70, CD80, CD86, CD123, CD133, CD137, CD137L, CD160, CD272, CEACAM5, CLEC9, CLEC91, CRTH2, CSF-1, CSF-2, CSF-3, CXCL1, CXCL2, CXCL4, CXCL12, CXCL13, CXCR3, cMet, CTLA4, DLL3, DLL4, DNGR-1, E-cadherin, EGFR, ENTPD1, EpCAM, FCER1, FCER1A, FCER2, FGFR, FLAP, FOLH1, Gi24, GITR, GITRL, GPR5, GP100, GPRC5D, HER2, HER3, ICOSL, ICOS, HHLA2, HMGB1, HVEM, IDO, IFNa, IgE, IGF1R, IL2Rbeta, IL1, ILIA, IL1B, IL1F10, IL2, IL4, IL4Ra, IL5, IL5R, IL6, IL7, IL7Ra, IL8, IL9, IL9R, IL10, rhILlO, IL12, IL13, IL13Ra1, IL13Ra2, IL15, IL17, IL17Rb, IL18, IL22, IL23, IL25, IL7, IL33, IL35, ITGB4, ITK, KIR, LAG3, LAMP1, leptin, LPFS2, MHC class II, MUC-1, MUC-16, NCR3LG1, NKG2D, NKp46, NTPDase-1, OX40, OX40L, PD-1, PD-L1, PD-L2, PROM1, S152, SIRPalpha, SISP1, SLC, SPG64, ST2, STEAP1, STEAP2, Syk kinase, STEAP1, TROP2, TACI, TDO, TGFBETA, T14, TIGIT, TIM3, TLR, TLR2, TLR4, TLR5, TLR9, TMEF1, TNFa, TNFRSF7, Tp55, TREM1, TSLP, TSLPR, TWEAK, VEGF, VISTA, Vstm3, or WUCAM; VH2 is a second immunoglobulin heavy chain variable region that specifically binds to A2AR, APRIL, ATPDase, BAFF, BAFFR, BCMA, BlyS, BTK, BTLA, B7DC, B7H1, B7H2, B7H3, B7H4, B7H5, B7H6, B7H7, B7RP1, B7-4, C3, C5, CCL2, CCL3, CCL4, CCL5, CCL7, CCL8, CCL11, CCL15, CCL17, CCL19, CCL20, CCL21, CCL25, CCR3, CCR4, CD16A, CD19, CD20, CD24, CD27, CD28, CD30, CD38, CD39, CD40, CD40L, CD47, CD52, CD70, CD80, CD86, CD123, CD133, CD137, CD137L, CD160, CD272, CEACAM5, CLEC9, CLEC91, CRTH2, CSF-1, CSF-2, CSF-3, CXCL1, CXCL2, CXCL4, CXCL12, CXCL13, CXCR3, cMet, CTLA4, DLL3, DLL4, DNGR-1, E-cadherin, EGFR, ENTPD1, EpCAM, FCER1, FCER1A, FCER2, FGFR, FLAP, FOLH1, Gi24, GITR, GITRL, GPR5, GP100, GPRC5D, HER2, HER3, ICOSL, ICOS, HHLA2, HMGB1, HVEM, IDO, IFNa, IgE, IGF1R, IL2Rbeta, IL1, ILIA, IL1B, IL1F10, IL2, IL4, IL4Ra, IL5, IL5R, IL6, IL7, IL7Ra, IL8, IL9, IL9R, IL10, rhILlO, IL12, IL13, IL13Ra1, IL13Ra2, IL15, IL17, IL17Rb, IL18, IL22, IL23, IL25, IL7, IL33, IL35, ITGB4, ITK, KIR, LAG3, LAMP1, leptin, LPFS2, MHC class II, MUC-1, MUC-16, NCR3LG1, NKG2D, NKp46, NTPDase-1, OX40, OX40L, PD-1, PD-L1, PD-L2, PROM1, S152, SIRPalpha, SISP1, SLC, SPG64, ST2, STEAP1, STEAP2, Syk kinase, STEAP1, TROP2, TACI, TDO, TGFBETA, T14, TIGIT, TIM3, TLR, TLR2, TLR4, TLR5, TLR9, TMEF1, TNFa, TNFRSF7, Tp55, TREM1, TSLP, TSLPR, TWEAK, VEGF, VISTA, Vstm3, or WUCAM; VH3 is a third immunoglobulin heavy chain variable region that specifically binds to A2AR, APRIL, ATPDase, BAFF, BAFFR, BCMA, BlyS, BTK, BTLA, B7DC, B7H1, B7H2, B7H3, B7H4, B7H5, B7H6, B7H7, B7RP1, B7-4, C3, C5, CCL2, CCL3, CCL4, CCL5, CCL7, CCL8, CCL11, CCL15, CCL17, CCL19, CCL20, CCL21, CCL25, CCR3, CCR4, CD16A, CD19, CD20, CD24, CD27, CD28, CD30, CD38, CD39, CD40, CD40L, CD47, CD52, CD70, CD80, CD86, CD123, CD133, CD137, CD137L, CD160, CD272, CEACAM5, CLEC9, CLEC91, CRTH2, CSF-1, CSF-2, CSF-3, CXCL1, CXCL2, CXCL4, CXCL12, CXCL13, CXCR3, cMet, CTLA4, DLL3, DLL4, DNGR-1, E-cadherin, EGFR, ENTPD1, EpCAM, FCER1, FCER1A, FCER2, FGFR, FLAP, FOLH1, Gi24, GITR, GITRL, GPR5, GP100, GPRC5D, HER2, HER3, ICOSL, ICOS, HHLA2, HMGB1, HVEM, IDO, IFNa, IgE, IGF1R, IL2Rbeta, IL1, ILIA, IL1B, IL1F10, IL2, IL4, IL4Ra, IL5, IL5R, IL6, IL7, IL7Ra, IL8, IL9, IL9R, IL10, rhILlO, IL12, IL13, IL13Ra1, IL13Ra2, IL15, IL17, IL17Rb, IL18, IL22, IL23, IL25, IL7, IL33, IL35, ITGB4, ITK, KIR, LAG3, LAMP1, leptin, LPFS2, MHC class II, MUC-1, MUC-16, NCR3LG1, NKG2D, NKp46, NTPDase-1, OX40, OX40L, PD-1, PD-L1, PD-L2, PROM1, S152, SIRPalpha, SISP1, SLC, SPG64, ST2, STEAP1, STEAP2, Syk kinase, STEAP1, TROP2, TACI, TDO, TGFBETA, T14, TIGIT, TIM3, TLR, TLR2, TLR4, TLR5, TLR9, TMEF1, TNFa, TNFRSF7, Tp55, TREM1, TSLP, TSLPR, TWEAK, VEGF, VISTA, Vstm3, or WUCAM; VH4 is a fourth immunoglobulin heavy chain variable region that specifically binds to CD3; Fc is a region comprising an immunoglobulin heavy chain constant region 2 (CH2), an immunoglobulin heavy chain constant region 3 (CH3), and optionally, an immunoglobulin hinge; CH1 is an immunoglobulin heavy chain constant region 1; CL is an immunoglobulin light chain constant region; and L1-L88 are amino acid linkers. In some aspects, the first polypeptide has a structure represented by VL1-L1-VL2-L2-VH2-L3-VH1-L4-CH1-L5-Fc; VH1-L6-VH2-L7-VL2-L8-VL1-L9-CH1-L10-Fc; VL1-L11-VL2-L12-VH2-L13-VH1-L14-CL-L15-Fc; VH1-L16-VH2-L17-VL2-L18-VL1-L19-CL-L20-Fc; VL1-L21-VL2-L22-VH2-L23-VH1-L24-CH1-L25-CL-L26-Fc; VH1-L27-VH2-L28-VL2-L29-VL1-L30-CH1-L31-CL-L32-Fc; VL1-L33-VL2-L34-VH2-L35-VH1-L36-CL-L37-CH1-L38-Fc; or VH1-L39-VH2-L40-VL2-L41-VL1-L42-CL-L43-CH1-L44-Fc and the second polypeptide has a structure represented by VL3-L45-VL4-L46-VH4-L47-VH3-L48-CH1-L49-Fc. In some aspects, the first polypeptide has a structure represented by VL1-L1-VL2-L2-VH2-L3-VH1-L4-CH1-L5-Fc; VH1-L6-VH2-L7-VL2-L8-VL1-L9-CH1-L10-Fc; VL1-L11-VL2-L12-VH2-L13-VH1-L14-CL-L15-Fc; VH1-L16-VH2-L17-VL2-L18-VL1-L19-CL-L20-Fc; VL1-L21-VL2-L22-VH2-L23-VH1-L24-CH1-L25-CL-L26-Fc; VH1-L27-VH2-L28-VL2-L29-VL1-L30-CH1-L31-CL-L32-Fc; VL1-L33-VL2-L34-VH2-L35-VH1-L36-CL-L37-CH1-L38-Fc; or VH1-L39-VH2-L40-VL2-L41-VL1-L42-CL-L43-CH1-L44-Fc and the second polypeptide has a structure represented by VH3-L50-VH4-L51-VL4-L52-VL3-L53-CH1-L54-Fc. In some aspects, the first polypeptide has a structure represented by VL1-L1-VL2-L2-VH2-L3-VH1-L4-CH1-L5-Fc; VH1-L6-VH2-L7-VL2-L8-VL1-L9-CH1-L10-Fc; VL1-L11-VL2-L12-VH2-L13-VH1-L14-CL-L15-Fc; VH1-L16-VH2-L17-VL2-L18-VL1-L19-CL-L20-Fc; VL1-L21-VL2-L22-VH2-L23-VH1-L24-CH1-L25-CL-L26-Fc; VH1-L27-VH2-L28-VL2-L29-VL1-L30-CH1-L31-CL-L32-Fc; VL1-L33-VL2-L34-VH2-L35-VH1-L36-CL-L37-CH1-L38-Fc; or VH1-L39-VH2-L40-VL2-L41-VL1-L42-CL-L43-CH1-L44-Fc and the second polypeptide has a structure represented by VL3-L55-VL4-L56-VH4-L57-VH3-L58-CL-L59-Fc. In some aspects, the first polypeptide has a structure represented by VL1-L1-VL2-L2-VH2-L3-VH1-L4-CH1-L5-Fc; VH1-L6-VH2-L7-VL2-L8-VL1-L9-CH1-L10-Fc; VL1-L11-VL2-L12-VH2-L13-VH1-L14-CL-L15-Fc; VH1-L16-VH2-L17-VL2-L18-VL1-L19-CL-L20-Fc; VL1-L21-VL2-L22-VH2-L23-VH1-L24-CH1-L25-CL-L26-Fc; VH1-L27-VH2-L28-VL2-L29-VL1-L30-CH1-L31-CL-L32-Fc; VL1-L33-VL2-L34-VH2-L35-VH1-L36-CL-L37-CH1-L38-Fc; or VH1-L39-VH2-L40-VL2-L41-VL1-L42-CL-L43-CH1-L44-Fc and the second polypeptide has a structure represented by VH3-L60-VH4-L61-VL4-L62-VL3-L63-CL-L64-Fc. In some aspects, the first polypeptide has a structure represented by VL1-L1-VL2-L2-VH2-L3-VH1-L4-CH1-L5-Fc; VH1-L6-VH2-L7-VL2-L8-VL1-L9-CH1-L10-Fc; VL1-L11-VL2-L12-VH2-L13-VH1-L14-CL-L15-Fc; VH1-L16-VH2-L17-VL2-L18-VL1-L19-CL-L20-Fc; VL1-L21-VL2-L22-VH2-L23-VH1-L24-CH1-L25-CL-L26-Fc; VH1-L27-VH2-L28-VL2-L29-VL1-L30-CH1-L31-CL-L32-Fc; VL1-L33-VL2-L34-VH2-L35-VH1-L36-CL-L37-CH1-L38-Fc; or VH1-L39-VH2-L40-VL2-L41-VL1-L42-CL-L43-CH1-L44-Fc and the second polypeptide has a structure represented by VL3-L65-VL4-L66-VH4-L67-VH3-L68-CH1-L69-CL-L70-Fc. In some aspects, the first polypeptide has a structure represented by VL1-L1-VL2-L2-VH2-L3-VH1-L4-CH1-L5-Fc; VH1-L6-VH2-L7-VL2-L8-VL1-L9-CH1-L10-Fc; VL1-L11-VL2-L12-VH2-L13-VH1-L14-CL-L15-Fc; VH1-L16-VH2-L17-VL2-L18-VL1-L19-CL-L20-Fc; VL1-L21-VL2-L22-VH2-L23-VH1-L24-CH1-L25-CL-L26-Fc; VH1-L27-VH2-L28-VL2-L29-VL1-L30-CH1-L31-CL-L32-Fc; VL1-L33-VL2-L34-VH2-L35-VH1-L36-CL-L37-CH1-L38-Fc; or VH1-L39-VH2-L40-VL2-L41-VL1-L42-CL-L43-CH1-L44-Fc and the second polypeptide has a structure represented by VH3-L71-VH4-L72-VL4-L73-VL3-L74-CH1-L75-CL-L76-Fc. In some aspects, the first polypeptide has a structure represented by VL1-L1-VL2-L2-VH2-L3-VH1-L4-CH1-L5-Fc; VH1-L6-VH2-L7-VL2-L8-VL1-L9-CH1-L10-Fc; VL1-L11-VL2-L12-VH2-L13-VH1-L14-CL-L15-Fc; VH1-L16-VH2-L17-VL2-L18-VL1-L19-CL-L20-Fc; VL1-L21-VL2-L22-VH2-L23-VH1-L24-CH1-L25-CL-L26-Fc; VH1-L27-VH2-L28-VL2-L29-VL1-L30-CH1-L31-CL-L32-Fc; VL1-L33-VL2-L34-VH2-L35-VH1-L36-CL-L37-CH1-L38-Fc; or VH1-L39-VH2-L40-VL2-L41-VL1-L42-CL-L43-CH1-L44-Fc and the second polypeptide has a structure represented by VL3-L77-VL4-L78-VH4-L79-VH3-L80-CL-L81-CH1-L82-Fc. In some aspects, the first polypeptide has a structure represented by VL1-L1-VL2-L2-VH2-L3-VH1-L4-CH1-L5-Fc; VH1-L6-VH2-L7-VL2-L8-VL1-L9-CH1-L10-Fc; VL1-L11-VL2-L12-VH2-L13-VH1-L14-CL-L15-Fc; VH1-L16-VH2-L17-VL2-L18-VL1-L19-CL-L20-Fc; VL1-L21-VL2-L22-VH2-L23-VH1-L24-CH1-L25-CL-L26-Fc; VH1-L27-VH2-L28-VL2-L29-VL1-L30-CH1-L31-CL-L32-Fc; VL1-L33-VL2-L34-VH2-L35-VH1-L36-CL-L37-CH1-L38-Fc; or VH1-L39-VH2-L40-VL2-L41-VL1-L42-CL-L43-CH1-L44-Fc and the second polypeptide has a structure represented by VH3-L83-VH4-L84-VL4-L85-VL3-L86-CL-L87-CH1-L88-Fc. In some aspects, VL1 and VH1 may specifically bind to A2AR, APRIL, ATPDase, BAFF, BAFFR, BCMA, BlyS, BTK, BTLA, B7DC, B7H1, B7H2, B7H3, B7H4, B7H5, B7H6, B7H7, B7RP1, B7-4, C3, C5, CCL2, CCL3, CCL4, CCL5, CCL7, CCL8, CCL11, CCL15, CCL17, CCL19, CCL20, CCL21, CCL25, CCR3, CCR4, CD16A, CD19, CD20, CD24, CD27, CD28, CD30, CD38, CD39, CD40, CD40L, CD47, CD52, CD70, CD80, CD86, CD123, CD133, CD137, CD137L, CD160, CD272, CEACAM5, CLEC9, CLEC91, CRTH2, CSF-1, CSF-2, CSF-3, CXCL1, CXCL2, CXCL4, CXCL12, CXCL13, CXCR3, cMet, CTLA4, DLL3, DLL4, DNGR-1, E-cadherin, EGFR, ENTPD1, EpCAM, FCER1, FCER1A, FCER2, FGFR, FLAP, FOLH1, Gi24, GITR, GITRL, GPR5, GP100, GPRC5D, HER2, HER3, ICOSL, ICOS, HHLA2, HMGB1, HVEM, IDO, IFNa, IgE, IGF1R, IL2Rbeta, IL1, ILIA, IL1B, IL1F10, IL2, IL4, IL4Ra, IL5, IL5R, IL6, IL7, IL7Ra, IL8, IL9, IL9R, IL10, rhILlO, IL12, IL13, IL13Ra1, IL13Ra2, IL15, IL17, IL17Rb, IL18, IL22, IL23, IL25, IL7, IL33, IL35, ITGB4, ITK, KIR, LAG3, LAMP1, leptin, LPFS2, MHC class II, MUC-1, MUC-16, NCR3LG1, NKG2D, NKp46, NTPDase-1, OX40, OX40L, PD-1, PD-L1, PD-L2, PROM1, S152, SIRPalpha, SISP1, SLC, SPG64, ST2, STEAP1, STEAP2, Syk kinase, STEAP1, TROP2, TACI, TDO, TGFBETA, T14, TIGIT, TIM3, TLR, TLR2, TLR4, TLR5, TLR9, TMEF1, TNFa, TNFRSF7, Tp55, TREM1, TSLP, TSLPR, TWEAK, VEGF, VISTA, Vstm3, or WUCAM. In some aspects, VL2 and VH2 may specifically bind to A2AR, APRIL, ATPDase, BAFF, BAFFR, BCMA, BlyS, BTK, BTLA, B7DC, B7H1, B7H2, B7H3, B7H4, B7H5, B7H6, B7H7, B7RP1, B7-4, C3, C5, CCL2, CCL3, CCL4, CCL5, CCL7, CCL8, CCL11, CCL15, CCL17, CCL19, CCL20, CCL21, CCL25, CCR3, CCR4, CD16A, CD19, CD20, CD24, CD27, CD28, CD30, CD38, CD39, CD40, CD40L, CD47, CD52, CD70, CD80, CD86, CD123, CD133, CD137, CD137L, CD160, CD272, CEACAM5, CLEC9, CLEC91, CRTH2, CSF-1, CSF-2, CSF-3, CXCL1, CXCL2, CXCL4, CXCL12, CXCL13, CXCR3, cMet, CTLA4, DLL3, DLL4, DNGR-1, E-cadherin, EGFR, ENTPD1, EpCAM, FCER1, FCER1A, FCER2, FGFR, FLAP, FOLH1, Gi24, GITR, GITRL, GPR5, GP100, GPRC5D, HER2, HER3, ICOSL, ICOS, HHLA2, HMGB1, HVEM, IDO, IFNa, IgE, IGF1R, IL2Rbeta, IL1, ILIA, IL1B, IL1F10, IL2, IL4, IL4Ra, IL5, IL5R, IL6, IL7, IL7Ra, IL8, IL9, IL9R, IL10, rhILlO, IL12, IL13, IL13Ra1, IL13Ra2, IL15, IL17, IL17Rb, IL18, IL22, IL23, IL25, IL7, IL33, IL35, ITGB4, ITK, KIR, LAG3, LAMP1, leptin, LPFS2, MHC class II, MUC-1, MUC-16, NCR3LG1, NKG2D, NKp46, NTPDase-1, OX40, OX40L, PD-1, PD-L1, PD-L2, PROM1, S152, SIRPalpha, SISP1, SLC, SPG64, ST2, STEAP1, STEAP2, Syk kinase, STEAP1, TROP2, TACI, TDO, TGFBETA, T14, TIGIT, TIM3, TLR, TLR2, TLR4, TLR5, TLR9, TMEF1, TNFa, TNFRSF7, Tp55, TREM1, TSLP, TSLPR, TWEAK, VEGF, VISTA, Vstm3, or WUCAM. In some aspects, VL3 and VH3 may specifically bind to A2AR, APRIL, ATPDase, BAFF, BAFFR, BCMA, BlyS, BTK, BTLA, B7DC, B7H1, B7H2, B7H3, B7H4, B7H5, B7H6, B7H7, B7RP1, B7-4, C3, C5, CCL2, CCL3, CCL4, CCL5, CCL7, CCL8, CCL11, CCL15, CCL17, CCL19, CCL20, CCL21, CCL25, CCR3, CCR4, CD16A, CD19, CD20, CD24, CD27, CD28, CD30, CD38, CD39, CD40, CD40L, CD47, CD52, CD70, CD80, CD86, CD123, CD133, CD137, CD137L, CD160, CD272, CEACAM5, CLEC9, CLEC91, CRTH2, CSF-1, CSF-2, CSF-3, CXCL1, CXCL2, CXCL4, CXCL12, CXCL13, CXCR3, cMet, CTLA4, DLL3, DLL4, DNGR-1, E-cadherin, EGFR, ENTPD1, EpCAM, FCER1, FCER1A, FCER2, FGFR, FLAP, FOLH1, Gi24, GITR, GITRL, GPR5, GP100, GPRC5D, HER2, HER3, ICOSL, ICOS, HHLA2, HMGB1, HVEM, IDO, IFNa, IgE, IGF1R, IL2Rbeta, IL1, ILIA, IL1B, IL1F10, IL2, IL4, IL4Ra, IL5, IL5R, IL6, IL7, IL7Ra, IL8, IL9, IL9R, IL10, rhILlO, IL12, IL13, IL13Ra1, IL13Ra2, IL15, IL17, IL17Rb, IL18, IL22, IL23, IL25, IL7, IL33, IL35, ITGB4, ITK, KIR, LAG3, LAMP1, leptin, LPFS2, MHC class II, MUC-1, MUC-16, NCR3LG1, NKG2D, NKp46, NTPDase-1, OX40, OX40L, PD-1, PD-L1, PD-L2, PROM1, S152, SIRPalpha, SISP1, SLC, SPG64, ST2, STEAP1, STEAP2, Syk kinase, STEAP1, TROP2, TACI, TDO, TGFBETA, T14, TIGIT, TIM3, TLR, TLR2, TLR4, TLR5, TLR9, TMEF1, TNFa, TNFRSF7, Tp55, TREM1, TSLP, TSLPR, TWEAK, VEGF, VISTA, Vstm3, or WUCAM. The VL1, VL2, VL3, VH1, VH2, and/or VH3 may specifically bind to CD28, cMet, Trop2, CD20, or CD19; and VL4 and/or VH4 may specifically bind to CD3. For example, the antigen binding polypeptide complex may specifically bind CD28, CD3, cMet, and Trop2. For example, the antigen binding polypeptide complex may specifically bind CD28, CD3, CD19, and CD20. For example, VL1 and VH1 may specifically bind to CD28. For example, VL2 and VH2 may specifically bind to CD28. For example, VL3 and VH3 may specifically bind to CD28. For example, VL1 and VH1 may specifically bind to cMet. For example, VL2 and VH2 may specifically bind to cMet. For example, VL3 and VH3 may specifically bind to cMet. For example, VL1 and VH1 may specifically bind to Trop2. For example, VL2 and VH2 may specifically bind to Trop2. For example, VL3 and VH3 may specifically bind to Trop2. For example, VL1 and VH1 may specifically bind to CD19. For example, VL2 and VH2 may specifically bind to CD19. For example, VL3 and VH3 may specifically bind to CD19. For example, VL1 and VH1 may specifically bind to CD20. For example, VL2 and VH2 may specifically bind to CD20. For example, VL3 and VH3 may specifically bind to CD20.

In some aspects, the invention is directed to an antigen binding polypeptide complex comprising a first polypeptide and a second polypeptide; wherein the first polypeptide has a structure represented by VL1-L1-VL2-L2-VH2-L3-VH1-L4-CH1-L5-Fc; VH1-L6-VH2-L7-VL2-L8-VL1-L9-CH1-L10-Fc; VL1-L11-VL2-L12-VH2-L13-VH1-L14-CL-L15-Fc; VH1-L16-VH2-L17-VL2-L18-VL1-L19-CL-L20-Fc; VL1-L21-VL2-L22-VH2-L23-VH1-L24-CH1-L25-CL-L26-Fc; VH1-L27-VH2-L28-VL2-L29-VL1-L30-CH1-L31-CL-L32-Fc; VL1-L33-VL2-L34-VH2-L35-VH1-L36-CL-L37-CH1-L38-Fc; or VH1-L39-VH2-L40-VL2-L41-VL1-L42-CL-L43-CH1-L44-Fc; wherein the second polypeptide has a structure represented by VL3-L45-VL4-L46-VH4-L47-VH3-L48-CH1-L49-Fc; VH3-L50-VH4-L51-VL4-L52-VL3-L53-CH1-L54-Fc; VL3-L55-VL4-L56-VH4-L57-VH3-L58-CL-L59-Fc; VH3-L60-VH4-L61-VL4-L62-VL3-L63-CL-L64-Fc; VL3-L65-VL4-L66-VH4-L67-VH3-L68-CH1-L69-CL-L70-Fc; VH3-L71-VH4-L72-VL4-L73-VL3-L74-CH1-L75-CL-L76-Fc; VL3-L77-VL4-L78-VH4-L79-VH3-L80-CL-L81-CH1-L82-Fc; or VH3-L83-VH4-L84-VL4-L85-VL3-L86-CL-L87-CH1-L88-Fc; wherein VL1 is a first immunoglobulin light chain variable region that specifically binds to CD28; VL2 is a second immunoglobulin light chain variable region that specifically binds to A2AR, APRIL, ATPDase, BAFF, BAFFR, BCMA, BlyS, BTK, BTLA, B7DC, B7H1, B7H2, B7H3, B7H4, B7H5, B7H6, B7H7, B7RP1, B7-4, C3, C5, CCL2, CCL3, CCL4, CCL5, CCL7, CCL8, CCL11, CCL15, CCL17, CCL19, CCL20, CCL21, CCL25, CCR3, CCR4, CD16A, CD19, CD20, CD24, CD27, CD28, CD30, CD38, CD39, CD40, CD40L, CD47, CD52, CD70, CD80, CD86, CD123, CD133, CD137, CD137L, CD160, CD272, CEACAM5, CLEC9, CLEC91, CRTH2, CSF-1, CSF-2, CSF-3, CXCL1, CXCL2, CXCL4, CXCL12, CXCL13, CXCR3, cMet, CTLA4, DLL3, DLL4, DNGR-1, E-cadherin, EGFR, ENTPD1, EpCAM, FCER1, FCER1A, FCER2, FGFR, FLAP, FOLH1, Gi24, GITR, GITRL, GPR5, GP100, GPRC5D, HER2, HER3, ICOSL, ICOS, HHLA2, HMGB1, HVEM, IDO, IFNa, IgE, IGF1R, IL2Rbeta, IL1, ILIA, IL1B, IL1F10, IL2, IL4, IL4Ra, IL5, IL5R, IL6, IL7, IL7Ra, IL8, IL9, IL9R, IL10, rhILlO, IL12, IL13, IL13Ra1, IL13Ra2, IL15, IL17, IL17Rb, IL18, IL22, IL23, IL25, IL7, IL33, IL35, ITGB4, ITK, KIR, LAG3, LAMP1, leptin, LPFS2, MHC class II, MUC-1, MUC-16, NCR3LG1, NKG2D, NKp46, NTPDase-1, OX40, OX40L, PD-1, PD-L1, PD-L2, PROM1, S152, SIRPalpha, SISP1, SLC, SPG64, ST2, STEAP1, STEAP2, Syk kinase, STEAP1, TROP2, TACI, TDO, TGFBETA, T14, TIGIT, TIM3, TLR, TLR2, TLR4, TLR5, TLR9, TMEF1, TNFa, TNFRSF7, Tp55, TREM1, TSLP, TSLPR, TWEAK, VEGF, VISTA, Vstm3, or WUCAM; VL3 is a third immunoglobulin light chain variable region that specifically binds to A2AR, APRIL, ATPDase, BAFF, BAFFR, BCMA, BlyS, BTK, BTLA, B7DC, B7H1, B7H2, B7H3, B7H4, B7H5, B7H6, B7H7, B7RP1, B7-4, C3, C5, CCL2, CCL3, CCL4, CCL5, CCL7, CCL8, CCL11, CCL15, CCL17, CCL19, CCL20, CCL21, CCL25, CCR3, CCR4, CD16A, CD19, CD20, CD24, CD27, CD28, CD30, CD38, CD39, CD40, CD40L, CD47, CD52, CD70, CD80, CD86, CD123, CD133, CD137, CD137L, CD160, CD272, CEACAM5, CLEC9, CLEC91, CRTH2, CSF-1, CSF-2, CSF-3, CXCL1, CXCL2, CXCL4, CXCL12, CXCL13, CXCR3, cMet, CTLA4, DLL3, DLL4, DNGR-1, E-cadherin, EGFR, ENTPD1, EpCAM, FCER1, FCER1A, FCER2, FGFR, FLAP, FOLH1, Gi24, GITR, GITRL, GPR5, GP100, GPRC5D, HER2, HER3, ICOSL, ICOS, HHLA2, HMGB1, HVEM, IDO, IFNa, IgE, IGF1R, IL2Rbeta, IL1, ILIA, IL1B, IL1F10, IL2, IL4, IL4Ra, IL5, IL5R, IL6, IL7, IL7Ra, IL8, IL9, IL9R, IL10, rhILlO, IL12, IL13, IL13Ra1, IL13Ra2, IL15, IL17, IL17Rb, IL18, IL22, IL23, IL25, IL7, IL33, IL35, ITGB4, ITK, KIR, LAG3, LAMP1, leptin, LPFS2, MHC class II, MUC-1, MUC-16, NCR3LG1, NKG2D, NKp46, NTPDase-1, OX40, OX40L, PD-1, PD-L1, PD-L2, PROM1, S152, SIRPalpha, SISP1, SLC, SPG64, ST2, STEAP1, STEAP2, Syk kinase, STEAP1, TROP2, TACI, TDO, TGFBETA, T14, TIGIT, TIM3, TLR, TLR2, TLR4, TLR5, TLR9, TMEF1, TNFa, TNFRSF7, Tp55, TREM1, TSLP, TSLPR, TWEAK, VEGF, VISTA, Vstm3, or WUCAM; VL4 is a fourth immunoglobulin light chain variable region that specifically binds to CD3; VH1 is a first immunoglobulin heavy chain variable region that specifically binds to CD28; VH2 is a second immunoglobulin heavy chain variable region that specifically binds to A2AR, APRIL, ATPDase, BAFF, BAFFR, BCMA, BlyS, BTK, BTLA, B7DC, B7H1, B7H2, B7H3, B7H4, B7H5, B7H6, B7H7, B7RP1, B7-4, C3, C5, CCL2, CCL3, CCL4, CCL5, CCL7, CCL8, CCL11, CCL15, CCL17, CCL19, CCL20, CCL21, CCL25, CCR3, CCR4, CD16A, CD19, CD20, CD24, CD27, CD28, CD30, CD38, CD39, CD40, CD40L, CD47, CD52, CD70, CD80, CD86, CD123, CD133, CD137, CD137L, CD160, CD272, CEACAM5, CLEC9, CLEC91, CRTH2, CSF-1, CSF-2, CSF-3, CXCL1, CXCL2, CXCL4, CXCL12, CXCL13, CXCR3, cMet, CTLA4, DLL3, DLL4, DNGR-1, E-cadherin, EGFR, ENTPD1, EpCAM, FCER1, FCER1A, FCER2, FGFR, FLAP, FOLH1, Gi24, GITR, GITRL, GPR5, GP100, GPRC5D, HER2, HER3, ICOSL, ICOS, HHLA2, HMGB1, HVEM, IDO, IFNa, IgE, IGF1R, IL2Rbeta, IL1, ILIA, IL1B, IL1F10, IL2, IL4, IL4Ra, IL5, IL5R, IL6, IL7, IL7Ra, IL8, IL9, IL9R, IL10, rhILlO, IL12, IL13, IL13Ra1, IL13Ra2, IL15, IL17, IL17Rb, IL18, IL22, IL23, IL25, IL7, IL33, IL35, ITGB4, ITK, KIR, LAG3, LAMP1, leptin, LPFS2, MHC class II, MUC-1, MUC-16, NCR3LG1, NKG2D, NKp46, NTPDase-1, OX40, OX40L, PD-1, PD-L1, PD-L2, PROM1, S152, SIRPalpha, SISP1, SLC, SPG64, ST2, STEAP1, STEAP2, Syk kinase, STEAP1, TROP2, TACI, TDO, TGFBETA, T14, TIGIT, TIM3, TLR, TLR2, TLR4, TLR5, TLR9, TMEF1, TNFa, TNFRSF7, Tp55, TREM1, TSLP, TSLPR, TWEAK, VEGF, VISTA, Vstm3, or WUCAM; VH3 is a third immunoglobulin heavy chain variable region that specifically binds to A2AR, APRIL, ATPDase, BAFF, BAFFR, BCMA, BlyS, BTK, BTLA, B7DC, B7H1, B7H2, B7H3, B7H4, B7H5, B7H6, B7H7, B7RP1, B7-4, C3, C5, CCL2, CCL3, CCL4, CCL5, CCL7, CCL8, CCL11, CCL15, CCL17, CCL19, CCL20, CCL21, CCL25, CCR3, CCR4, CD16A, CD19, CD20, CD24, CD27, CD28, CD30, CD38, CD39, CD40, CD40L, CD47, CD52, CD70, CD80, CD86, CD123, CD133, CD137, CD137L, CD160, CD272, CEACAM5, CLEC9, CLEC91, CRTH2, CSF-1, CSF-2, CSF-3, CXCL1, CXCL2, CXCL4, CXCL12, CXCL13, CXCR3, cMet, CTLA4, DLL3, DLL4, DNGR-1, E-cadherin, EGFR, ENTPD1, EpCAM, FCER1, FCER1A, FCER2, FGFR, FLAP, FOLH1, Gi24, GITR, GITRL, GPR5, GP100, GPRC5D, HER2, HER3, ICOSL, ICOS, HHLA2, HMGB1, HVEM, IDO, IFNa, IgE, IGF1R, IL2Rbeta, IL1, ILIA, IL1B, IL1F10, IL2, IL4, IL4Ra, IL5, IL5R, IL6, IL7, IL7Ra, IL8, IL9, IL9R, IL10, rhILlO, IL12, IL13, IL13Ra1, IL13Ra2, IL15, IL17, IL17Rb, IL18, IL22, IL23, IL25, IL7, IL33, IL35, ITGB4, ITK, KIR, LAG3, LAMP1, leptin, LPFS2, MHC class II, MUC-1, MUC-16, NCR3LG1, NKG2D, NKp46, NTPDase-1, OX40, OX40L, PD-1, PD-L1, PD-L2, PROM1, S152, SIRPalpha, SISP1, SLC, SPG64, ST2, STEAP1, STEAP2, Syk kinase, STEAP1, TROP2, TACI, TDO, TGFBETA, T14, TIGIT, TIM3, TLR, TLR2, TLR4, TLR5, TLR9, TMEF1, TNFa, TNFRSF7, Tp55, TREM1, TSLP, TSLPR, TWEAK, VEGF, VISTA, Vstm3, or WUCAM; VH4 is a fourth immunoglobulin heavy chain variable region that specifically binds to CD3; Fe is a region comprising an immunoglobulin heavy chain constant region 2 (CH2), an immunoglobulin heavy chain constant region 3 (CH3), and optionally, an immunoglobulin hinge; CH1 is an immunoglobulin heavy chain constant region 1; CL is an immunoglobulin light chain constant region; and L1-L88 are amino acid linkers. In some aspects, the first polypeptide has a structure represented by VL1-L1-VL2-L2-VH2-L3-VH1-L4-CH1-L5-Fc; VH1-L6-VH2-L7-VL2-L8-VL1-L9-CH1-L10-Fc; VL1-L11-VL2-L12-VH2-L13-VH1-L14-CL-L15-Fc; VH1-L16-VH2-L17-VL2-L18-VL1-L19-CL-L20-Fc; VL1-L21-VL2-L22-VH2-L23-VH1-L24-CH1-L25-CL-L26-Fc; VH1-L27-VH2-L28-VL2-L29-VL1-L30-CH1-L31-CL-L32-Fc; VL1-L33-VL2-L34-VH2-L35-VH1-L36-CL-L37-CH1-L38-Fc; or VH1-L39-VH2-L40-VL2-L41-VL1-L42-CL-L43-CH1-L44-Fc and the second polypeptide has a structure represented by VL3-L45-VL4-L46-VH4-L47-VH3-L48-CH1-L49-Fc. In some aspects, the first polypeptide has a structure represented by VL1-L1-VL2-L2-VH2-L3-VH1-L4-CH1-L5-Fc; VH1-L6-VH2-L7-VL2-L8-VL1-L9-CH1-L10-Fc; VL1-L11-VL2-L12-VH2-L13-VH1-L14-CL-L15-Fc; VH1-L16-VH2-L17-VL2-L18-VL1-L19-CL-L20-Fc; VL1-L21-VL2-L22-VH2-L23-VH1-L24-CH1-L25-CL-L26-Fc; VH1-L27-VH2-L28-VL2-L29-VL1-L30-CH1-L31-CL-L32-Fc; VL1-L33-VL2-L34-VH2-L35-VH1-L36-CL-L37-CH1-L38-Fc; or VH1-L39-VH2-L40-VL2-L41-VL1-L42-CL-L43-CH1-L44-Fc and the second polypeptide has a structure represented by VH3-L50-VH4-L51-VL4-L52-VL3-L53-CH1-L54-Fc. In some aspects, the first polypeptide has a structure represented by VL1-L1-VL2-L2-VH2-L3-VH1-L4-CH1-L5-Fc; VH1-L6-VH2-L7-VL2-L8-VL1-L9-CH1-L10-Fc; VL1-L11-VL2-L12-VH2-L13-VH1-L14-CL-L15-Fc; VH1-L16-VH2-L17-VL2-L18-VL1-L19-CL-L20-Fc; VL1-L21-VL2-L22-VH2-L23-VH1-L24-CH1-L25-CL-L26-Fc; VH1-L27-VH2-L28-VL2-L29-VL1-L30-CH1-L31-CL-L32-Fc; VL1-L33-VL2-L34-VH2-L35-VH1-L36-CL-L37-CH1-L38-Fc; or VH1-L39-VH2-L40-VL2-L41-VL1-L42-CL-L43-CH1-L44-Fc and the second polypeptide has a structure represented by VL3-L55-VL4-L56-VH4-L57-VH3-L58-CL-L59-Fc. In some aspects, the first polypeptide has a structure represented by VL1-L1-VL2-L2-VH2-L3-VH1-L4-CH1-L5-Fc; VH1-L6-VH2-L7-VL2-L8-VL1-L9-CH1-L10-Fc; VL1-L11-VL2-L12-VH2-L13-VH1-L14-CL-L15-Fc; VH1-L16-VH2-L17-VL2-L18-VL1-L19-CL-L20-Fc; VL1-L21-VL2-L22-VH2-L23-VH1-L24-CH1-L25-CL-L26-Fc; VH1-L27-VH2-L28-VL2-L29-VL1-L30-CH1-L31-CL-L32-Fc; VL1-L33-VL2-L34-VH2-L35-VH1-L36-CL-L37-CH1-L38-Fc; or VH1-L39-VH2-L40-VL2-L41-VL1-L42-CL-L43-CH1-L44-Fc and the second polypeptide has a structure represented by VH3-L60-VH4-L61-VL4-L62-VL3-L63-CL-L64-Fc. In some aspects, the first polypeptide has a structure represented by VL1-L1-VL2-L2-VH2-L3-VH1-L4-CH1-L5-Fc; VH1-L6-VH2-L7-VL2-L8-VL1-L9-CH1-L10-Fc; VL1-L11-VL2-L12-VH2-L13-VH1-L14-CL-L15-Fc; VH1-L16-VH2-L17-VL2-L18-VL1-L19-CL-L20-Fc; VL1-L21-VL2-L22-VH2-L23-VH1-L24-CH1-L25-CL-L26-Fc; VH1-L27-VH2-L28-VL2-L29-VL1-L30-CH1-L31-CL-L32-Fc; VL1-L33-VL2-L34-VH2-L35-VH1-L36-CL-L37-CH1-L38-Fc; or VH1-L39-VH2-L40-VL2-L41-VL1-L42-CL-L43-CH1-L44-Fc and the second polypeptide has a structure represented by VL3-L65-VL4-L66-VH4-L67-VH3-L68-CH1-L69-CL-L70-Fc. In some aspects, the first polypeptide has a structure represented by VL1-L1-VL2-L2-VH2-L3-VH1-L4-CH1-L5-Fc; VH1-L6-VH2-L7-VL2-L8-VL1-L9-CH1-L10-Fc; VL1-L11-VL2-L12-VH2-L13-VH1-L14-CL-L15-Fc; VH1-L16-VH2-L17-VL2-L18-VL1-L19-CL-L20-Fc; VL1-L21-VL2-L22-VH2-L23-VH1-L24-CH1-L25-CL-L26-Fc; VH1-L27-VH2-L28-VL2-L29-VL1-L30-CH1-L31-CL-L32-Fc; VL1-L33-VL2-L34-VH2-L35-VH1-L36-CL-L37-CH1-L38-Fc; or VH1-L39-VH2-L40-VL2-L41-VL1-L42-CL-L43-CH1-L44-Fc and the second polypeptide has a structure represented by VH3-L71-VH4-L72-VL4-L73-VL3-L74-CH1-L75-CL-L76-Fc. In some aspects, the first polypeptide has a structure represented by VL1-L1-VL2-L2-VH2-L3-VH1-L4-CH1-L5-Fc; VH1-L6-VH2-L7-VL2-L8-VL1-L9-CH1-L10-Fc; VL1-L11-VL2-L12-VH2-L13-VH1-L14-CL-L15-Fc; VH1-L16-VH2-L17-VL2-L18-VL1-L19-CL-L20-Fc; VL1-L21-VL2-L22-VH2-L23-VH1-L24-CH1-L25-CL-L26-Fc; VH1-L27-VH2-L28-VL2-L29-VL1-L30-CH1-L31-CL-L32-Fc; VL1-L33-VL2-L34-VH2-L35-VH1-L36-CL-L37-CH1-L38-Fc; or VH1-L39-VH2-L40-VL2-L41-VL1-L42-CL-L43-CH1-L44-Fc and the second polypeptide has a structure represented by VL3-L77-VL4-L78-VH4-L79-VH3-L80-CL-L81-CH1-L82-Fc. In some aspects, the first polypeptide has a structure represented by VL1-L1-VL2-L2-VH2-L3-VH1-L4-CH1-L5-Fc; VH1-L6-VH2-L7-VL2-L8-VL1-L9-CH1-L10-Fc; VL1-L11-VL2-L12-VH2-L13-VH1-L14-CL-L15-Fc; VH1-L16-VH2-L17-VL2-L18-VL1-L19-CL-L20-Fc; VL1-L21-VL2-L22-VH2-L23-VH1-L24-CH1-L25-CL-L26-Fc; VH1-L27-VH2-L28-VL2-L29-VL1-L30-CH1-L31-CL-L32-Fc; VL1-L33-VL2-L34-VH2-L35-VH1-L36-CL-L37-CH1-L38-Fc; or VH1-L39-VH2-L40-VL2-L41-VL1-L42-CL-L43-CH1-L44-Fc and the second polypeptide has a structure represented by VH3-L83-VH4-L84-VL4-L85-VL3-L86-CL-L87-CH1-L88-Fc. In some aspects, VL2 and VH2 specifically bind to A2AR, APRIL, ATPDase, BAFF, BAFFR, BCMA, BlyS, BTK, BTLA, B7DC, B7H1, B7H2, B7H3, B7H4, B7H5, B7H6, B7H7, B7RP1, B7-4, C3, C5, CCL2, CCL3, CCL4, CCL5, CCL7, CCL8, CCL11, CCL15, CCL17, CCL19, CCL20, CCL21, CCL25, CCR3, CCR4, CD16A, CD19, CD20, CD24, CD27, CD28, CD30, CD38, CD39, CD40, CD40L, CD47, CD52, CD70, CD80, CD86, CD123, CD133, CD137, CD137L, CD160, CD272, CEACAM5, CLEC9, CLEC91, CRTH2, CSF-1, CSF-2, CSF-3, CXCL1, CXCL2, CXCL4, CXCL12, CXCL13, CXCR3, cMet, CTLA4, DLL3, DLL4, DNGR-1, E-cadherin, EGFR, ENTPD1, EpCAM, FCER1, FCER1A, FCER2, FGFR, FLAP, FOLH1, Gi24, GITR, GITRL, GPR5, GP100, GPRC5D, HER2, HER3, ICOSL, ICOS, HHLA2, HMGB1, HVEM, IDO, IFNa, IgE, IGF1R, IL2Rbeta, IL1, ILIA, IL1B, IL1F10, IL2, IL4, IL4Ra, IL5, IL5R, IL6, IL7, IL7Ra, IL8, IL9, IL9R, IL10, rhILlO, IL12, IL13, IL13Ra1, IL13Ra2, IL15, IL17, IL17Rb, IL18, IL22, IL23, IL25, IL7, IL33, IL35, ITGB4, ITK, KIR, LAG3, LAMP1, leptin, LPFS2, MHC class II, MUC-1, MUC-16, NCR3LG1, NKG2D, NKp46, NTPDase-1, OX40, OX40L, PD-1, PD-L1, PD-L2, PROM1, S152, SIRPalpha, SISP1, SLC, SPG64, ST2, STEAP1, STEAP2, Syk kinase, STEAP1, TROP2, TACI, TDO, TGFBETA, T14, TIGIT, TIM3, TLR, TLR2, TLR4, TLR5, TLR9, TMEF1, TNFa, TNFRSF7, Tp55, TREM1, TSLP, TSLPR, TWEAK, VEGF, VISTA, Vstm3, or WUCAM. In some aspects, VL3 and VH3 specifically bind to A2AR, APRIL, ATPDase, BAFF, BAFFR, BCMA, BlyS, BTK, BTLA, B7DC, B7H1, B7H2, B7H3, B7H4, B7H5, B7H6, B7H7, B7RP1, B7-4, C3, C5, CCL2, CCL3, CCL4, CCL5, CCL7, CCL8, CCL11, CCL15, CCL17, CCL19, CCL20, CCL21, CCL25, CCR3, CCR4, CD16A, CD19, CD20, CD24, CD27, CD28, CD30, CD38, CD39, CD40, CD40L, CD47, CD52, CD70, CD80, CD86, CD123, CD133, CD137, CD137L, CD160, CD272, CEACAM5, CLEC9, CLEC91, CRTH2, CSF-1, CSF-2, CSF-3, CXCL1, CXCL2, CXCL4, CXCL12, CXCL13, CXCR3, cMet, CTLA4, DLL3, DLL4, DNGR-1, E-cadherin, EGFR, ENTPD1, EpCAM, FCER1, FCER1A, FCER2, FGFR, FLAP, FOLH1, Gi24, GITR, GITRL, GPR5, GP100, GPRC5D, HER2, HER3, ICOSL, ICOS, HHLA2, HMGB1, HVEM, IDO, IFNa, IgE, IGF1R, IL2Rbeta, IL1, ILIA, IL1B, IL1F10, IL2, IL4, IL4Ra, IL5, IL5R, IL6, IL7, IL7Ra, IL8, IL9, IL9R, IL10, rhILlO, IL12, IL13, IL13Ra1, IL13Ra2, IL15, IL17, IL17Rb, IL18, IL22, IL23, IL25, IL7, IL33, IL35, ITGB4, ITK, KIR, LAG3, LAMP1, leptin, LPFS2, MHC class II, MUC-1, MUC-16, NCR3LG1, NKG2D, NKp46, NTPDase-1, OX40, OX40L, PD-1, PD-L1, PD-L2, PROM1, S152, SIRPalpha, SISP1, SLC, SPG64, ST2, STEAP1, STEAP2, Syk kinase, STEAP1, TROP2, TACI, TDO, TGFBETA, T14, TIGIT, TIM3, TLR, TLR2, TLR4, TLR5, TLR9, TMEF1, TNFa, TNFRSF7, Tp55, TREM1, TSLP, TSLPR, TWEAK, VEGF, VISTA, Vstm3, or WUCAM. The VL2, VL3, VH2, and/or VH3 may specifically bind to CD28, cMet, Trop2, CD20, or CD19; VL1 and/or VH1 may specifically bind to CD28; and VL3 and/or VH3 may specifically bind to CD3. For example, the antigen binding polypeptide complex may specifically bind CD28, CD3, cMet, and Trop2. For example, the antigen binding polypeptide complex may specifically bind CD28, CD3, CD19, and CD20. For example, VL2 and VH2 may specifically bind to cMet. For example, VL3 and VH3 may specifically bind to cMet. For example, VL2 and VH2 may specifically bind to Trop2. For example, VL3 and VH3 may specifically bind to Trop2. For example, VL2 and VH2 may specifically bind to CD19. For example, VL3 and VH3 may specifically bind to CD19. For example, VL2 and VH2 may specifically bind to CD20. For example, VL3 and VH3 may specifically bind to CD20.

In some aspects, the invention is directed to an antigen binding polypeptide complex comprising a first polypeptide and a second polypeptide; wherein the first polypeptide has a structure represented by VL1-L1-VL2-L2-VH2-L3-VH1-L4-CH1-L5-Fc; VH1-L6-VH2-L7-VL2-L8-VL1-L9-CH1-L10-Fc; VL1-L11-VL2-L12-VH2-L13-VH1-L14-CL-L15-Fc; VH1-L16-VH2-L17-VL2-L18-VL1-L19-CL-L20-Fc; VL1-L21-VL2-L22-VH2-L23-VH1-L24-CH1-L25-CL-L26-Fc; VH1-L27-VH2-L28-VL2-L29-VL1-L30-CH1-L31-CL-L32-Fc; VL1-L33-VL2-L34-VH2-L35-VH1-L36-CL-L37-CH1-L38-Fc; or VH1-L39-VH2-L40-VL2-L41-VL1-L42-CL-L43-CH1-L44-Fc; wherein the second polypeptide has a structure represented by VL3-L45-VL4-L46-VH4-L47-VH3-L48-CH1-L49-Fc; VH3-L50-VH4-L51-VL4-L52-VL3-L53-CH1-L54-Fc; VL3-L55-VL4-L56-VH4-L57-VH3-L58-CL-L59-Fc; VH3-L60-VH4-L61-VL4-L62-VL3-L63-CL-L64-Fc; VL3-L65-VL4-L66-VH4-L67-VH3-L68-CH1-L69-CL-L70-Fc; VH3-L71-VH4-L72-VL4-L73-VL3-L74-CH1-L75-CL-L76-Fc; VL3-L77-VL4-L78-VH4-L79-VH3-L80-CL-L81-CH1-L82-Fc; or VH3-L83-VH4-L84-VL4-L85-VL3-L86-CL-L87-CH1-L88-Fc; wherein VL1 is a first immunoglobulin light chain variable region that specifically binds to A2AR, APRIL, ATPDase, BAFF, BAFFR, BCMA, BlyS, BTK, BTLA, B7DC, B7H1, B7H2, B7H3, B7H4, B7H5, B7H6, B7H7, B7RP1, B7-4, C3, C5, CCL2, CCL3, CCL4, CCL5, CCL7, CCL8, CCL11, CCL15, CCL17, CCL19, CCL20, CCL21, CCL25, CCR3, CCR4, CD16A, CD19, CD20, CD24, CD27, CD28, CD30, CD38, CD39, CD40, CD40L, CD47, CD52, CD70, CD80, CD86, CD123, CD133, CD137, CD137L, CD160, CD272, CEACAM5, CLEC9, CLEC91, CRTH2, CSF-1, CSF-2, CSF-3, CXCL1, CXCL2, CXCL4, CXCL12, CXCL13, CXCR3, cMet, CTLA4, DLL3, DLL4, DNGR-1, E-cadherin, EGFR, ENTPD1, EpCAM, FCER1, FCER1A, FCER2, FGFR, FLAP, FOLH1, Gi24, GITR, GITRL, GPR5, GP100, GPRC5D, HER2, HER3, ICOSL, ICOS, HHLA2, HMGB1, HVEM, IDO, IFNa, IgE, IGF1R, IL2Rbeta, IL1, ILIA, IL1B, IL1F10, IL2, IL4, IL4Ra, IL5, IL5R, IL6, IL7, IL7Ra, IL8, IL9, IL9R, IL10, rhILlO, IL12, IL13, IL13Ra1, IL13Ra2, IL15, IL17, IL17Rb, IL18, IL22, IL23, IL25, IL7, IL33, IL35, ITGB4, ITK, KIR, LAG3, LAMP1, leptin, LPFS2, MHC class II, MUC-1, MUC-16, NCR3LG1, NKG2D, NKp46, NTPDase-1, OX40, OX40L, PD-1, PD-L1, PD-L2, PROM1, S152, SIRPalpha, SISP1, SLC, SPG64, ST2, STEAP1, STEAP2, Syk kinase, STEAP1, TROP2, TACI, TDO, TGFBETA, T14, TIGIT, TIM3, TLR, TLR2, TLR4, TLR5, TLR9, TMEF1, TNFa, TNFRSF7, Tp55, TREM1, TSLP, TSLPR, TWEAK, VEGF, VISTA, Vstm3, or WUCAM; VL2 is a second immunoglobulin light chain variable region that specifically binds to CD28; VL3 is a third immunoglobulin light chain variable region that specifically binds to A2AR, APRIL, ATPDase, BAFF, BAFFR, BCMA, BlyS, BTK, BTLA, B7DC, B7H1, B7H2, B7H3, B7H4, B7H5, B7H6, B7H7, B7RP1, B7-4, C3, C5, CCL2, CCL3, CCL4, CCL5, CCL7, CCL8, CCL11, CCL15, CCL17, CCL19, CCL20, CCL21, CCL25, CCR3, CCR4, CD16A, CD19, CD20, CD24, CD27, CD28, CD30, CD38, CD39, CD40, CD40L, CD47, CD52, CD70, CD80, CD86, CD123, CD133, CD137, CD137L, CD160, CD272, CEACAM5, CLEC9, CLEC91, CRTH2, CSF-1, CSF-2, CSF-3, CXCL1, CXCL2, CXCL4, CXCL12, CXCL13, CXCR3, cMet, CTLA4, DLL3, DLL4, DNGR-1, E-cadherin, EGFR, ENTPD1, EpCAM, FCER1, FCER1A, FCER2, FGFR, FLAP, FOLH1, Gi24, GITR, GITRL, GPR5, GP100, GPRC5D, HER2, HER3, ICOSL, ICOS, HHLA2, HMGB1, HVEM, IDO, IFNa, IgE, IGF1R, IL2Rbeta, IL1, ILIA, IL1B, IL1F10, IL2, IL4, IL4Ra, IL5, IL5R, IL6, IL7, IL7Ra, IL8, IL9, IL9R, IL10, rhILlO, IL12, IL13, IL13Ra1, IL13Ra2, IL15, IL17, IL17Rb, IL18, IL22, IL23, IL25, IL7, IL33, IL35, ITGB4, ITK, KIR, LAG3, LAMP1, leptin, LPFS2, MHC class II, MUC-1, MUC-16, NCR3LG1, NKG2D, NKp46, NTPDase-1, OX40, OX40L, PD-1, PD-L1, PD-L2, PROM1, S152, SIRPalpha, SISP1, SLC, SPG64, ST2, STEAP1, STEAP2, Syk kinase, STEAP1, TROP2, TACI, TDO, TGFBETA, T14, TIGIT, TIM3, TLR, TLR2, TLR4, TLR5, TLR9, TMEF1, TNFa, TNFRSF7, Tp55, TREM1, TSLP, TSLPR, TWEAK, VEGF, VISTA, Vstm3, or WUCAM; VL4 is a fourth immunoglobulin light chain variable region that specifically binds to CD3; VH1 is a first immunoglobulin heavy chain variable region that specifically binds to A2AR, APRIL, ATPDase, BAFF, BAFFR, BCMA, BlyS, BTK, BTLA, B7DC, B7H1, B7H2, B7H3, B7H4, B7H5, B7H6, B7H7, B7RP1, B7-4, C3, C5, CCL2, CCL3, CCL4, CCL5, CCL7, CCL8, CCL11, CCL15, CCL17, CCL19, CCL20, CCL21, CCL25, CCR3, CCR4, CD16A, CD19, CD20, CD24, CD27, CD28, CD30, CD38, CD39, CD40, CD40L, CD47, CD52, CD70, CD80, CD86, CD123, CD133, CD137, CD137L, CD160, CD272, CEACAM5, CLEC9, CLEC91, CRTH2, CSF-1, CSF-2, CSF-3, CXCL1, CXCL2, CXCL4, CXCL12, CXCL13, CXCR3, cMet, CTLA4, DLL3, DLL4, DNGR-1, E-cadherin, EGFR, ENTPD1, EpCAM, FCER1, FCER1A, FCER2, FGFR, FLAP, FOLH1, Gi24, GITR, GITRL, GPR5, GP100, GPRC5D, HER2, HER3, ICOSL, ICOS, HHLA2, HMGB1, HVEM, IDO, IFNa, IgE, IGF1R, IL2Rbeta, IL1, ILIA, IL1B, IL1F10, IL2, IL4, IL4Ra, IL5, IL5R, IL6, IL7, IL7Ra, IL8, IL9, IL9R, IL10, rhILlO, IL12, IL13, IL13Ra1, IL13Ra2, IL15, IL17, IL17Rb, IL18, IL22, IL23, IL25, IL7, IL33, IL35, ITGB4, ITK, KIR, LAG3, LAMP1, leptin, LPFS2, MHC class II, MUC-1, MUC-16, NCR3LG1, NKG2D, NKp46, NTPDase-1, OX40, OX40L, PD-1, PD-L1, PD-L2, PROM1, S152, SIRPalpha, SISP1, SLC, SPG64, ST2, STEAP1, STEAP2, Syk kinase, STEAP1, TROP2, TACI, TDO, TGFBETA, T14, TIGIT, TIM3, TLR, TLR2, TLR4, TLR5, TLR9, TMEF1, TNFa, TNFRSF7, Tp55, TREM1, TSLP, TSLPR, TWEAK, VEGF, VISTA, Vstm3, or WUCAM; VH2 is a second immunoglobulin heavy chain variable region that specifically binds to CD28; VH3 is a third immunoglobulin heavy chain variable region that specifically binds to A2AR, APRIL, ATPDase, BAFF, BAFFR, BCMA, BlyS, BTK, BTLA, B7DC, B7H1, B7H2, B7H3, B7H4, B7H5, B7H6, B7H7, B7RP1, B7-4, C3, C5, CCL2, CCL3, CCL4, CCL5, CCL7, CCL8, CCL11, CCL15, CCL17, CCL19, CCL20, CCL21, CCL25, CCR3, CCR4, CD16A, CD19, CD20, CD24, CD27, CD28, CD30, CD38, CD39, CD40, CD40L, CD47, CD52, CD70, CD80, CD86, CD123, CD133, CD137, CD137L, CD160, CD272, CEACAM5, CLEC9, CLEC91, CRTH2, CSF-1, CSF-2, CSF-3, CXCL1, CXCL2, CXCL4, CXCL12, CXCL13, CXCR3, cMet, CTLA4, DLL3, DLL4, DNGR-1, E-cadherin, EGFR, ENTPD1, EpCAM, FCER1, FCER1A, FCER2, FGFR, FLAP, FOLH1, Gi24, GITR, GITRL, GPR5, GP100, GPRC5D, HER2, HER3, ICOSL, ICOS, HHLA2, HMGB1, HVEM, IDO, IFNa, IgE, IGF1R, IL2Rbeta, IL1, ILIA, IL1B, IL1F10, IL2, IL4, IL4Ra, IL5, IL5R, IL6, IL7, IL7Ra, IL8, IL9, IL9R, IL10, rhILlO, IL12, IL13, IL13Ra1, IL13Ra2, IL15, IL17, IL17Rb, IL18, IL22, IL23, IL25, IL7, IL33, IL35, ITGB4, ITK, KIR, LAG3, LAMP1, leptin, LPFS2, MHC class II, MUC-1, MUC-16, NCR3LG1, NKG2D, NKp46, NTPDase-1, OX40, OX40L, PD-1, PD-L1, PD-L2, PROM1, S152, SIRPalpha, SISP1, SLC, SPG64, ST2, STEAP1, STEAP2, Syk kinase, STEAP1, TROP2, TACI, TDO, TGFBETA, T14, TIGIT, TIM3, TLR, TLR2, TLR4, TLR5, TLR9, TMEF1, TNFa, TNFRSF7, Tp55, TREM1, TSLP, TSLPR, TWEAK, VEGF, VISTA, Vstm3, or WUCAM; VH4 is a fourth immunoglobulin heavy chain variable region that specifically binds to CD3; Fc is a region comprising an immunoglobulin heavy chain constant region 2 (CH2), an immunoglobulin heavy chain constant region 3 (CH3), and optionally, an immunoglobulin hinge; CH1 is an immunoglobulin heavy chain constant region 1; CL is an immunoglobulin light chain constant region; and L1-L88 are amino acid linkers. In some aspects, the first polypeptide has a structure represented by VL1-L1-VL2-L2-VH2-L3-VH1-L4-CH1-L5-Fc; VH1-L6-VH2-L7-VL2-L8-VL1-L9-CH1-L10-Fc; VL1-L11-VL2-L12-VH2-L13-VH1-L14-CL-L15-Fc; VH1-L16-VH2-L17-VL2-L18-VL1-L19-CL-L20-Fc; VL1-L21-VL2-L22-VH2-L23-VH1-L24-CH1-L25-CL-L26-Fc; VH1-L27-VH2-L28-VL2-L29-VL1-L30-CH1-L31-CL-L32-Fc; VL1-L33-VL2-L34-VH2-L35-VH1-L36-CL-L37-CH1-L38-Fc; or VH1-L39-VH2-L40-VL2-L41-VL1-L42-CL-L43-CH1-L44-Fc and the second polypeptide has a structure represented by VL3-L45-VL4-L46-VH4-L47-VH3-L48-CH1-L49-Fc. In some aspects, the first polypeptide has a structure represented by VL1-L1-VL2-L2-VH2-L3-VH1-L4-CH1-L5-Fc; VH1-L6-VH2-L7-VL2-L8-VL1-L9-CH1-L10-Fc; VL1-L11-VL2-L12-VH2-L13-VH1-L14-CL-L15-Fc; VH1-L16-VH2-L17-VL2-L18-VL1-L19-CL-L20-Fc; VL1-L21-VL2-L22-VH2-L23-VH1-L24-CH1-L25-CL-L26-Fc; VH1-L27-VH2-L28-VL2-L29-VL1-L30-CH1-L31-CL-L32-Fc; VL1-L33-VL2-L34-VH2-L35-VH1-L36-CL-L37-CH1-L38-Fc; or VH1-L39-VH2-L40-VL2-L41-VL1-L42-CL-L43-CH1-L44-Fc and the second polypeptide has a structure represented by VH3-L50-VH4-L51-VL4-L52-VL3-L53-CH1-L54-Fc. In some aspects, the first polypeptide has a structure represented by VL1-L1-VL2-L2-VH2-L3-VH1-L4-CH1-L5-Fc; VH1-L6-VH2-L7-VL2-L8-VL1-L9-CH1-L10-Fc; VL1-L11-VL2-L12-VH2-L13-VH1-L14-CL-L15-Fc; VH1-L16-VH2-L17-VL2-L18-VL1-L19-CL-L20-Fc; VL1-L21-VL2-L22-VH2-L23-VH1-L24-CH1-L25-CL-L26-Fc; VH1-L27-VH2-L28-VL2-L29-VL1-L30-CH1-L31-CL-L32-Fc; VL1-L33-VL2-L34-VH2-L35-VH1-L36-CL-L37-CH1-L38-Fc; or VH1-L39-VH2-L40-VL2-L41-VL1-L42-CL-L43-CH1-L44-Fc and the second polypeptide has a structure represented by VL3-L55-VL4-L56-VH4-L57-VH3-L58-CL-L59-Fc. In some aspects, the first polypeptide has a structure represented by VL1-L1-VL2-L2-VH2-L3-VH1-L4-CH1-L5-Fc; VH1-L6-VH2-L7-VL2-L8-VL1-L9-CH1-L10-Fc; VL1-L11-VL2-L12-VH2-L13-VH1-L14-CL-L15-Fc; VH1-L16-VH2-L17-VL2-L18-VL1-L19-CL-L20-Fc; VL1-L21-VL2-L22-VH2-L23-VH1-L24-CH1-L25-CL-L26-Fc; VH1-L27-VH2-L28-VL2-L29-VL1-L30-CH1-L31-CL-L32-Fc; VL1-L33-VL2-L34-VH2-L35-VH1-L36-CL-L37-CH1-L38-Fc; or VH1-L39-VH2-L40-VL2-L41-VL1-L42-CL-L43-CH1-L44-Fc and the second polypeptide has a structure represented by VH3-L60-VH4-L61-VL4-L62-VL3-L63-CL-L64-Fc. In some aspects, the first polypeptide has a structure represented by VL1-L1-VL2-L2-VH2-L3-VH1-L4-CH1-L5-Fc; VH1-L6-VH2-L7-VL2-L8-VL1-L9-CH1-L10-Fc; VL1-L11-VL2-L12-VH2-L13-VH1-L14-CL-L15-Fc; VH1-L16-VH2-L17-VL2-L18-VL1-L19-CL-L20-Fc; VL1-L21-VL2-L22-VH2-L23-VH1-L24-CH1-L25-CL-L26-Fc; VH1-L27-VH2-L28-VL2-L29-VL1-L30-CH1-L31-CL-L32-Fc; VL1-L33-VL2-L34-VH2-L35-VH1-L36-CL-L37-CH1-L38-Fc; or VH1-L39-VH2-L40-VL2-L41-VL1-L42-CL-L43-CH1-L44-Fc and the second polypeptide has a structure represented by VL3-L65-VL4-L66-VH4-L67-VH3-L68-CH1-L69-CL-L70-Fc. In some aspects, the first polypeptide has a structure represented by VL1-L1-VL2-L2-VH2-L3-VH1-L4-CH1-L5-Fc; VH1-L6-VH2-L7-VL2-L8-VL1-L9-CH1-L10-Fc; VL1-L11-VL2-L12-VH2-L13-VH1-L14-CL-L15-Fc; VH1-L16-VH2-L17-VL2-L18-VL1-L19-CL-L20-Fc; VL1-L21-VL2-L22-VH2-L23-VH1-L24-CH1-L25-CL-L26-Fc; VH1-L27-VH2-L28-VL2-L29-VL1-L30-CH1-L31-CL-L32-Fc; VL1-L33-VL2-L34-VH2-L35-VH1-L36-CL-L37-CH1-L38-Fc; or VH1-L39-VH2-L40-VL2-L41-VL1-L42-CL-L43-CH1-L44-Fc and the second polypeptide has a structure represented by VH3-L71-VH4-L72-VL4-L73-VL3-L74-CH1-L75-CL-L76-Fc. In some aspects, the first polypeptide has a structure represented by VL1-L1-VL2-L2-VH2-L3-VH1-L4-CH1-L5-Fc; VH1-L6-VH2-L7-VL2-L8-VL1-L9-CH1-L10-Fc; VL1-L11-VL2-L12-VH2-L13-VH1-L14-CL-L15-Fc; VH1-L16-VH2-L17-VL2-L18-VL1-L19-CL-L20-Fc; VL1-L21-VL2-L22-VH2-L23-VH1-L24-CH1-L25-CL-L26-Fc; VH1-L27-VH2-L28-VL2-L29-VL1-L30-CH1-L31-CL-L32-Fc; VL1-L33-VL2-L34-VH2-L35-VH1-L36-CL-L37-CH1-L38-Fc; or VH1-L39-VH2-L40-VL2-L41-VL1-L42-CL-L43-CH1-L44-Fc and the second polypeptide has a structure represented by VL3-L77-VL4-L78-VH4-L79-VH3-L80-CL-L81-CH1-L82-Fc. In some aspects, the first polypeptide has a structure represented by VL1-L1-VL2-L2-VH2-L3-VH1-L4-CH1-L5-Fc; VH1-L6-VH2-L7-VL2-L8-VL1-L9-CH1-L10-Fc; VL1-L11-VL2-L12-VH2-L13-VH1-L14-CL-L15-Fc; VH1-L16-VH2-L17-VL2-L18-VL1-L19-CL-L20-Fc; VL1-L21-VL2-L22-VH2-L23-VH1-L24-CH1-L25-CL-L26-Fc; VH1-L27-VH2-L28-VL2-L29-VL1-L30-CH1-L31-CL-L32-Fc; VL1-L33-VL2-L34-VH2-L35-VH1-L36-CL-L37-CH1-L38-Fc; or VH1-L39-VH2-L40-VL2-L41-VL1-L42-CL-L43-CH1-L44-Fc and the second polypeptide has a structure represented by VH3-L83-VH4-L84-VL4-L85-VL3-L86-CL-L87-CH1-L88-Fc. In some aspects, VL1 and VH1 specifically bind to A2AR, APRIL, ATPDase, BAFF, BAFFR, BCMA, BlyS, BTK, BTLA, B7DC, B7H1, B7H2, B7H3, B7H4, B7H5, B7H6, B7H7, B7RP1, B7-4, C3, C5, CCL2, CCL3, CCL4, CCL5, CCL7, CCL8, CCL11, CCL15, CCL17, CCL19, CCL20, CCL21, CCL25, CCR3, CCR4, CD16A, CD19, CD20, CD24, CD27, CD28, CD30, CD38, CD39, CD40, CD40L, CD47, CD52, CD70, CD80, CD86, CD123, CD133, CD137, CD137L, CD160, CD272, CEACAM5, CLEC9, CLEC91, CRTH2, CSF-1, CSF-2, CSF-3, CXCL1, CXCL2, CXCL4, CXCL12, CXCL13, CXCR3, cMet, CTLA4, DLL3, DLL4, DNGR-1, E-cadherin, EGFR, ENTPD1, EpCAM, FCER1, FCER1A, FCER2, FGFR, FLAP, FOLH1, Gi24, GITR, GITRL, GPR5, GP100, GPRC5D, HER2, HER3, ICOSL, ICOS, HHLA2, HMGB1, HVEM, IDO, IFNa, IgE, IGF1R, IL2Rbeta, IL1, ILIA, IL1B, IL1F10, IL2, IL4, IL4Ra, IL5, IL5R, IL6, IL7, IL7Ra, IL8, IL9, IL9R, IL10, rhILlO, IL12, IL13, IL13Ra1, IL13Ra2, IL15, IL17, IL17Rb, IL18, IL22, IL23, IL25, IL7, IL33, IL35, ITGB4, ITK, KIR, LAG3, LAMP1, leptin, LPFS2, MHC class II, MUC-1, MUC-16, NCR3LG1, NKG2D, NKp46, NTPDase-1, OX40, OX40L, PD-1, PD-L1, PD-L2, PROM1, S152, SIRPalpha, SISP1, SLC, SPG64, ST2, STEAP1, STEAP2, Syk kinase, STEAP1, TROP2, TACI, TDO, TGFBETA, T14, TIGIT, TIM3, TLR, TLR2, TLR4, TLR5, TLR9, TMEF1, TNFa, TNFRSF7, Tp55, TREM1, TSLP, TSLPR, TWEAK, VEGF, VISTA, Vstm3, or WUCAM. In some aspects, VL3 and VH3 specifically bind to A2AR, APRIL, ATPDase, BAFF, BAFFR, BCMA, BlyS, BTK, BTLA, B7DC, B7H1, B7H2, B7H3, B7H4, B7H5, B7H6, B7H7, B7RP1, B7-4, C3, C5, CCL2, CCL3, CCL4, CCL5, CCL7, CCL8, CCL11, CCL15, CCL17, CCL19, CCL20, CCL21, CCL25, CCR3, CCR4, CD16A, CD19, CD20, CD24, CD27, CD28, CD30, CD38, CD39, CD40, CD40L, CD47, CD52, CD70, CD80, CD86, CD123, CD133, CD137, CD137L, CD160, CD272, CEACAM5, CLEC9, CLEC91, CRTH2, CSF-1, CSF-2, CSF-3, CXCL1, CXCL2, CXCL4, CXCL12, CXCL13, CXCR3, cMet, CTLA4, DLL3, DLL4, DNGR-1, E-cadherin, EGFR, ENTPD1, EpCAM, FCER1, FCER1A, FCER2, FGFR, FLAP, FOLH1, Gi24, GITR, GITRL, GPR5, GP100, GPRC5D, HER2, HER3, ICOSL, ICOS, HHLA2, HMGB1, HVEM, IDO, IFNa, IgE, IGF1R, IL2Rbeta, IL1, ILIA, IL1B, IL1F10, IL2, IL4, IL4Ra, IL5, IL5R, IL6, IL7, IL7Ra, IL8, IL9, IL9R, IL10, rhILlO, IL12, IL13, IL13Ra1, IL13Ra2, IL15, IL17, IL17Rb, IL18, IL22, IL23, IL25, IL7, IL33, IL35, ITGB4, ITK, KIR, LAG3, LAMP1, leptin, LPFS2, MHC class II, MUC-1, MUC-16, NCR3LG1, NKG2D, NKp46, NTPDase-1, OX40, OX40L, PD-1, PD-L1, PD-L2, PROM1, S152, SIRPalpha, SISP1, SLC, SPG64, ST2, STEAP1, STEAP2, Syk kinase, STEAP1, TROP2, TACI, TDO, TGFBETA, T14, TIGIT, TIM3, TLR, TLR2, TLR4, TLR5, TLR9, TMEF1, TNFa, TNFRSF7, Tp55, TREM1, TSLP, TSLPR, TWEAK, VEGF, VISTA, Vstm3, or WUCAM. The VL1, VL3, VH1, and/or VH3 may specifically bind to CD28, cMet, Trop2, CD20, or CD19; VL2 and/or VH2 may specifically bind to CD28; and VL4 and/or VH4 may specifically bind to CD3. For example, the antigen binding polypeptide complex may specifically bind CD28, CD3, cMet, and Trop2. For example, the antigen binding polypeptide complex may specifically bind CD28, CD3, CD19, and CD20. For example, VL1 and VH1 may specifically bind to cMet. For example, VL3 and VH3 may specifically bind to cMet. For example, VL1 and VH1 may specifically bind to Trop2. For example, VL3 and VH3 may specifically bind to Trop2. For example, VL1 and VH1 may specifically bind to CD19. For example, VL3 and VH3 may specifically bind to CD19. For example, VL1 and VH1 may specifically bind to CD20. For example, VL3 and VH3 may specifically bind to CD20.

In some aspects, the invention is directed to an antigen binding polypeptide complex comprising a first polypeptide and a second polypeptide; wherein the first polypeptide has a structure represented by VL1-L1-VL2-L2-VH2-L3-VH1-L4-CH1-L5-Fc; VH1-L6-VH2-L7-VL2-L8-VL1-L9-CH1-L10-Fc; VL1-L11-VL2-L12-VH2-L13-VH1-L14-CL-L15-Fc; VH1-L16-VH2-L17-VL2-L18-VL1-L19-CL-L20-Fc; VL1-L21-VL2-L22-VH2-L23-VH1-L24-CH1-L25-CL-L26-Fc; VH1-L27-VH2-L28-VL2-L29-VL1-L30-CH1-L31-CL-L32-Fc; VL1-L33-VL2-L34-VH2-L35-VH1-L36-CL-L37-CH1-L38-Fc; or VH1-L39-VH2-L40-VL2-L41-VL1-L42-CL-L43-CH1-L44-Fc; wherein the second polypeptide has a structure represented by VL3-L45-VL4-L46-VH4-L47-VH3-L48-CH1-L49-Fc; VH3-L50-VH4-L51-VL4-L52-VL3-L53-CH1-L54-Fc; VL3-L55-VL4-L56-VH4-L57-VH3-L58-CL-L59-Fc; VH3-L60-VH4-L61-VL4-L62-VL3-L63-CL-L64-Fc; VL3-L65-VL4-L66-VH4-L67-VH3-L68-CH1-L69-CL-L70-Fc; VH3-L71-VH4-L72-VL4-L73-VL3-L74-CH1-L75-CL-L76-Fc; VL3-L77-VL4-L78-VH4-L79-VH3-L80-CL-L81-CH1-L82-Fc; or VH3-L83-VH4-L84-VL4-L85-VL3-L86-CL-L87-CH1-L88-Fc; wherein VL1 is a first immunoglobulin light chain variable region that specifically binds to A2AR, APRIL, ATPDase, BAFF, BAFFR, BCMA, BlyS, BTK, BTLA, B7DC, B7H1, B7H2, B7H3, B7H4, B7H5, B7H6, B7H7, B7RP1, B7-4, C3, C5, CCL2, CCL3, CCL4, CCL5, CCL7, CCL8, CCL11, CCL15, CCL17, CCL19, CCL20, CCL21, CCL25, CCR3, CCR4, CD16A, CD19, CD20, CD24, CD27, CD28, CD30, CD38, CD39, CD40, CD40L, CD47, CD52, CD70, CD80, CD86, CD123, CD133, CD137, CD137L, CD160, CD272, CEACAM5, CLEC9, CLEC91, CRTH2, CSF-1, CSF-2, CSF-3, CXCL1, CXCL2, CXCL4, CXCL12, CXCL13, CXCR3, cMet, CTLA4, DLL3, DLL4, DNGR-1, E-cadherin, EGFR, ENTPD1, EpCAM, FCER1, FCER1A, FCER2, FGFR, FLAP, FOLH1, Gi24, GITR, GITRL, GPR5, GP100, GPRC5D, HER2, HER3, ICOSL, ICOS, HHLA2, HMGB1, HVEM, IDO, IFNa, IgE, IGF1R, IL2Rbeta, IL1, ILIA, IL1B, IL1F10, IL2, IL4, IL4Ra, IL5, IL5R, IL6, IL7, IL7Ra, IL8, IL9, IL9R, IL10, rhILlO, IL12, IL13, IL13Ra1, IL13Ra2, IL15, IL17, IL17Rb, IL18, IL22, IL23, IL25, IL7, IL33, IL35, ITGB4, ITK, KIR, LAG3, LAMP1, leptin, LPFS2, MHC class II, MUC-1, MUC-16, NCR3LG1, NKG2D, NKp46, NTPDase-1, OX40, OX40L, PD-1, PD-L1, PD-L2, PROM1, S152, SIRPalpha, SISP1, SLC, SPG64, ST2, STEAP1, STEAP2, Syk kinase, STEAP1, TROP2, TACI, TDO, TGFBETA, T14, TIGIT, TIM3, TLR, TLR2, TLR4, TLR5, TLR9, TMEF1, TNFa, TNFRSF7, Tp55, TREM1, TSLP, TSLPR, TWEAK, VEGF, VISTA, Vstm3, or WUCAM; VL2 is a second immunoglobulin light chain variable region that specifically binds to A2AR, APRIL, ATPDase, BAFF, BAFFR, BCMA, BlyS, BTK, BTLA, B7DC, B7H1, B7H2, B7H3, B7H4, B7H5, B7H6, B7H7, B7RP1, B7-4, C3, C5, CCL2, CCL3, CCL4, CCL5, CCL7, CCL8, CCL11, CCL15, CCL17, CCL19, CCL20, CCL21, CCL25, CCR3, CCR4, CD16A, CD19, CD20, CD24, CD27, CD28, CD30, CD38, CD39, CD40, CD40L, CD47, CD52, CD70, CD80, CD86, CD123, CD133, CD137, CD137L, CD160, CD272, CEACAM5, CLEC9, CLEC91, CRTH2, CSF-1, CSF-2, CSF-3, CXCL1, CXCL2, CXCL4, CXCL12, CXCL13, CXCR3, cMet, CTLA4, DLL3, DLL4, DNGR-1, E-cadherin, EGFR, ENTPD1, EpCAM, FCER1, FCER1A, FCER2, FGFR, FLAP, FOLH1, Gi24, GITR, GITRL, GPR5, GP100, GPRC5D, HER2, HER3, ICOSL, ICOS, HHLA2, HMGB1, HVEM, IDO, IFNa, IgE, IGF1R, IL2Rbeta, IL1, ILIA, IL1B, IL1F10, IL2, IL4, IL4Ra, IL5, IL5R, IL6, IL7, IL7Ra, IL8, IL9, IL9R, IL10, rhILlO, IL12, IL13, IL13Ra1, IL13Ra2, IL15, IL17, IL17Rb, IL18, IL22, IL23, IL25, IL7, IL33, IL35, ITGB4, ITK, KIR, LAG3, LAMP1, leptin, LPFS2, MHC class II, MUC-1, MUC-16, NCR3LG1, NKG2D, NKp46, NTPDase-1, OX40, OX40L, PD-1, PD-L1, PD-L2, PROM1, S152, SIRPalpha, SISP1, SLC, SPG64, ST2, STEAP1, STEAP2, Syk kinase, STEAP1, TROP2, TACI, TDO, TGFBETA, T14, TIGIT, TIM3, TLR, TLR2, TLR4, TLR5, TLR9, TMEF1, TNFa, TNFRSF7, Tp55, TREM1, TSLP, TSLPR, TWEAK, VEGF, VISTA, Vstm3, or WUCAM; VL3 is a third immunoglobulin light chain variable region that specifically binds to CD28; VL4 is a fourth immunoglobulin light chain variable region that specifically binds to CD3; VH1 is a first immunoglobulin heavy chain variable region that specifically binds to A2AR, APRIL, ATPDase, BAFF, BAFFR, BCMA, BlyS, BTK, BTLA, B7DC, B7H1, B7H2, B7H3, B7H4, B7H5, B7H6, B7H7, B7RP1, B7-4, C3, C5, CCL2, CCL3, CCL4, CCL5, CCL7, CCL8, CCL11, CCL15, CCL17, CCL19, CCL20, CCL21, CCL25, CCR3, CCR4, CD16A, CD19, CD20, CD24, CD27, CD28, CD30, CD38, CD39, CD40, CD40L, CD47, CD52, CD70, CD80, CD86, CD123, CD133, CD137, CD137L, CD160, CD272, CEACAM5, CLEC9, CLEC91, CRTH2, CSF-1, CSF-2, CSF-3, CXCL1, CXCL2, CXCL4, CXCL12, CXCL13, CXCR3, cMet, CTLA4, DLL3, DLL4, DNGR-1, E-cadherin, EGFR, ENTPD1, EpCAM, FCER1, FCER1A, FCER2, FGFR, FLAP, FOLH1, Gi24, GITR, GITRL, GPR5, GP100, GPRC5D, HER2, HER3, ICOSL, ICOS, HHLA2, HMGB1, HVEM, IDO, IFNa, IgE, IGF1R, IL2Rbeta, IL1, ILIA, IL1B, IL1F10, IL2, IL4, IL4Ra, IL5, IL5R, IL6, IL7, IL7Ra, IL8, IL9, IL9R, IL10, rhILlO, IL12, IL13, IL13Ra1, IL13Ra2, IL15, IL17, IL17Rb, IL18, IL22, IL23, IL25, IL7, IL33, IL35, ITGB4, ITK, KIR, LAG3, LAMP1, leptin, LPFS2, MHC class II, MUC-1, MUC-16, NCR3LG1, NKG2D, NKp46, NTPDase-1, OX40, OX40L, PD-1, PD-L1, PD-L2, PROM1, S152, SIRPalpha, SISP1, SLC, SPG64, ST2, STEAP1, STEAP2, Syk kinase, STEAP1, TROP2, TACI, TDO, TGFBETA, T14, TIGIT, TIM3, TLR, TLR2, TLR4, TLR5, TLR9, TMEF1, TNFa, TNFRSF7, Tp55, TREM1, TSLP, TSLPR, TWEAK, VEGF, VISTA, Vstm3, or WUCAM; VH2 is a second immunoglobulin heavy chain variable region that specifically binds to A2AR, APRIL, ATPDase, BAFF, BAFFR, BCMA, BlyS, BTK, BTLA, B7DC, B7H1, B7H2, B7H3, B7H4, B7H5, B7H6, B7H7, B7RP1, B7-4, C3, C5, CCL2, CCL3, CCL4, CCL5, CCL7, CCL8, CCL11, CCL15, CCL17, CCL19, CCL20, CCL21, CCL25, CCR3, CCR4, CD16A, CD19, CD20, CD24, CD27, CD28, CD30, CD38, CD39, CD40, CD40L, CD47, CD52, CD70, CD80, CD86, CD123, CD133, CD137, CD137L, CD160, CD272, CEACAM5, CLEC9, CLEC91, CRTH2, CSF-1, CSF-2, CSF-3, CXCL1, CXCL2, CXCL4, CXCL12, CXCL13, CXCR3, cMet, CTLA4, DLL3, DLL4, DNGR-1, E-cadherin, EGFR, ENTPD1, EpCAM, FCER1, FCER1A, FCER2, FGFR, FLAP, FOLH1, Gi24, GITR, GITRL, GPR5, GP100, GPRC5D, HER2, HER3, ICOSL, ICOS, HHLA2, HMGB1, HVEM, IDO, IFNa, IgE, IGF1R, IL2Rbeta, IL1, ILIA, IL1B, IL1F10, IL2, IL4, IL4Ra, IL5, IL5R, IL6, IL7, IL7Ra, IL8, IL9, IL9R, IL10, rhILlO, IL12, IL13, IL13Ra1, IL13Ra2, IL15, IL17, IL17Rb, IL18, IL22, IL23, IL25, IL7, IL33, IL35, ITGB4, ITK, KIR, LAG3, LAMP1, leptin, LPFS2, MHC class II, MUC-1, MUC-16, NCR3LG1, NKG2D, NKp46, NTPDase-1, OX40, OX40L, PD-1, PD-L1, PD-L2, PROM1, S152, SIRPalpha, SISP1, SLC, SPG64, ST2, STEAP1, STEAP2, Syk kinase, STEAP1, TROP2, TACI, TDO, TGFBETA, T14, TIGIT, TIM3, TLR, TLR2, TLR4, TLR5, TLR9, TMEF1, TNFa, TNFRSF7, Tp55, TREM1, TSLP, TSLPR, TWEAK, VEGF, VISTA, Vstm3, or WUCAM; VH3 is a third immunoglobulin heavy chain variable region that specifically binds to CD28; VH4 is a fourth immunoglobulin heavy chain variable region that specifically binds to CD3; Fc is a region comprising an immunoglobulin heavy chain constant region 2 (CH2), an immunoglobulin heavy chain constant region 3 (CH3), and optionally, an immunoglobulin hinge; CH1 is an immunoglobulin heavy chain constant region 1; CL is an immunoglobulin light chain constant region; and L1-L88 are amino acid linkers. In some aspects, the first polypeptide has a structure represented by VL1-L1-VL2-L2-VH2-L3-VH1-L4-CH1-L5-Fc; VH1-L6-VH2-L7-VL2-L8-VL1-L9-CH1-L10-Fc; VL1-L11-VL2-L12-VH2-L13-VH1-L14-CL-L15-Fc; VH1-L16-VH2-L17-VL2-L18-VL1-L19-CL-L20-Fc; VL1-L21-VL2-L22-VH2-L23-VH1-L24-CH1-L25-CL-L26-Fc; VH1-L27-VH2-L28-VL2-L29-VL1-L30-CH1-L31-CL-L32-Fc; VL1-L33-VL2-L34-VH2-L35-VH1-L36-CL-L37-CH1-L38-Fc; or VH1-L39-VH2-L40-VL2-L41-VL1-L42-CL-L43-CH1-L44-Fc and the second polypeptide has a structure represented by VL3-L45-VL4-L46-VH4-L47-VH3-L48-CH1-L49-Fc. In some aspects, the first polypeptide has a structure represented by VL1-L1-VL2-L2-VH2-L3-VH1-L4-CH1-L5-Fc; VH1-L6-VH2-L7-VL2-L8-VL1-L9-CH1-L10-Fc; VL1-L11-VL2-L12-VH2-L13-VH1-L14-CL-L15-Fc; VH1-L16-VH2-L17-VL2-L18-VL1-L19-CL-L20-Fc; VL1-L21-VL2-L22-VH2-L23-VH1-L24-CH1-L25-CL-L26-Fc; VH1-L27-VH2-L28-VL2-L29-VL1-L30-CH1-L31-CL-L32-Fc; VL1-L33-VL2-L34-VH2-L35-VH1-L36-CL-L37-CH1-L38-Fc; or VH1-L39-VH2-L40-VL2-L41-VL1-L42-CL-L43-CH1-L44-Fc and the second polypeptide has a structure represented by VH3-L50-VH4-L51-VL4-L52-VL3-L53-CH1-L54-Fc. In some aspects, the first polypeptide has a structure represented by VL1-L1-VL2-L2-VH2-L3-VH1-L4-CH1-L5-Fc; VH1-L6-VH2-L7-VL2-L8-VL1-L9-CH1-L10-Fc; VL1-L11-VL2-L12-VH2-L13-VH1-L14-CL-L15-Fc; VH1-L16-VH2-L17-VL2-L18-VL1-L19-CL-L20-Fc; VL1-L21-VL2-L22-VH2-L23-VH1-L24-CH1-L25-CL-L26-Fc; VH1-L27-VH2-L28-VL2-L29-VL1-L30-CH1-L31-CL-L32-Fc; VL1-L33-VL2-L34-VH2-L35-VH1-L36-CL-L37-CH1-L38-Fc; or VH1-L39-VH2-L40-VL2-L41-VL1-L42-CL-L43-CH1-L44-Fc and the second polypeptide has a structure represented by VL3-L55-VL4-L56-VH4-L57-VH3-L58-CL-L59-Fc. In some aspects, the first polypeptide has a structure represented by VL1-L1-VL2-L2-VH2-L3-VH1-L4-CH1-L5-Fc; VH1-L6-VH2-L7-VL2-L8-VL1-L9-CH1-L10-Fc; VL1-L11-VL2-L12-VH2-L13-VH1-L14-CL-L15-Fc; VH1-L16-VH2-L17-VL2-L18-VL1-L19-CL-L20-Fc; VL1-L21-VL2-L22-VH2-L23-VH1-L24-CH1-L25-CL-L26-Fc; VH1-L27-VH2-L28-VL2-L29-VL1-L30-CH1-L31-CL-L32-Fc; VL1-L33-VL2-L34-VH2-L35-VH1-L36-CL-L37-CH1-L38-Fc; or VH1-L39-VH2-L40-VL2-L41-VL1-L42-CL-L43-CH1-L44-Fc and the second polypeptide has a structure represented by VH3-L60-VH4-L61-VL4-L62-VL3-L63-CL-L64-Fc. In some aspects, the first polypeptide has a structure represented by VL1-L1-VL2-L2-VH2-L3-VH1-L4-CH1-L5-Fc; VH1-L6-VH2-L7-VL2-L8-VL1-L9-CH1-L10-Fc; VL1-L11-VL2-L12-VH2-L13-VH1-L14-CL-L15-Fc; VH1-L16-VH2-L17-VL2-L18-VL1-L19-CL-L20-Fc; VL1-L21-VL2-L22-VH2-L23-VH1-L24-CH1-L25-CL-L26-Fc; VH1-L27-VH2-L28-VL2-L29-VL1-L30-CH1-L31-CL-L32-Fc; VL1-L33-VL2-L34-VH2-L35-VH1-L36-CL-L37-CH1-L38-Fc; or VH1-L39-VH2-L40-VL2-L41-VL1-L42-CL-L43-CH1-L44-Fc and the second polypeptide has a structure represented by VL3-L65-VL4-L66-VH4-L67-VH3-L68-CH1-L69-CL-L70-Fc. In some aspects, the first polypeptide has a structure represented by VL1-L1-VL2-L2-VH2-L3-VH1-L4-CH1-L5-Fc; VH1-L6-VH2-L7-VL2-L8-VL1-L9-CH1-L10-Fc; VL1-L11-VL2-L12-VH2-L13-VH1-L14-CL-L15-Fc; VH1-L16-VH2-L17-VL2-L18-VL1-L19-CL-L20-Fc; VL1-L21-VL2-L22-VH2-L23-VH1-L24-CH1-L25-CL-L26-Fc; VH1-L27-VH2-L28-VL2-L29-VL1-L30-CH1-L31-CL-L32-Fc; VL1-L33-VL2-L34-VH2-L35-VH1-L36-CL-L37-CH1-L38-Fc; or VH1-L39-VH2-L40-VL2-L41-VL1-L42-CL-L43-CH1-L44-Fc and the second polypeptide has a structure represented by VH3-L71-VH4-L72-VL4-L73-VL3-L74-CH1-L75-CL-L76-Fc. In some aspects, the first polypeptide has a structure represented by VL1-L1-VL2-L2-VH2-L3-VH1-L4-CH1-L5-Fc; VH1-L6-VH2-L7-VL2-L8-VL1-L9-CH1-L10-Fc; VL1-L11-VL2-L12-VH2-L13-VH1-L14-CL-L15-Fc; VH1-L16-VH2-L17-VL2-L18-VL1-L19-CL-L20-Fc; VL1-L21-VL2-L22-VH2-L23-VH1-L24-CH1-L25-CL-L26-Fc; VH1-L27-VH2-L28-VL2-L29-VL1-L30-CH1-L31-CL-L32-Fc; VL1-L33-VL2-L34-VH2-L35-VH1-L36-CL-L37-CH1-L38-Fc; or VH1-L39-VH2-L40-VL2-L41-VL1-L42-CL-L43-CH1-L44-Fc and the second polypeptide has a structure represented by VL3-L77-VL4-L78-VH4-L79-VH3-L80-CL-L81-CH1-L82-Fc. In some aspects, the first polypeptide has a structure represented by VL1-L1-VL2-L2-VH2-L3-VH1-L4-CH1-L5-Fc; VH1-L6-VH2-L7-VL2-L8-VL1-L9-CH1-L10-Fc; VL1-L11-VL2-L12-VH2-L13-VH1-L14-CL-L15-Fc; VH1-L16-VH2-L17-VL2-L18-VL1-L19-CL-L20-Fc; VL1-L21-VL2-L22-VH2-L23-VH1-L24-CH1-L25-CL-L26-Fc; VH1-L27-VH2-L28-VL2-L29-VL1-L30-CH1-L31-CL-L32-Fc; VL1-L33-VL2-L34-VH2-L35-VH1-L36-CL-L37-CH1-L38-Fc; or VH1-L39-VH2-L40-VL2-L41-VL1-L42-CL-L43-CH1-L44-Fc and the second polypeptide has a structure represented by VH3-L83-VH4-L84-VL4-L85-VL3-L86-CL-L87-CH1-L88-Fc. In some aspects, VL1 and VH1 specifically bind to A2AR, APRIL, ATPDase, BAFF, BAFFR, BCMA, BlyS, BTK, BTLA, B7DC, B7H1, B7H2, B7H3, B7H4, B7H5, B7H6, B7H7, B7RP1, B7-4, C3, C5, CCL2, CCL3, CCL4, CCL5, CCL7, CCL8, CCL11, CCL15, CCL17, CCL19, CCL20, CCL21, CCL25, CCR3, CCR4, CD16A, CD19, CD20, CD24, CD27, CD28, CD30, CD38, CD39, CD40, CD40L, CD47, CD52, CD70, CD80, CD86, CD123, CD133, CD137, CD137L, CD160, CD272, CEACAM5, CLEC9, CLEC91, CRTH2, CSF-1, CSF-2, CSF-3, CXCL1, CXCL2, CXCL4, CXCL12, CXCL13, CXCR3, cMet, CTLA4, DLL3, DLL4, DNGR-1, E-cadherin, EGFR, ENTPD1, EpCAM, FCER1, FCER1A, FCER2, FGFR, FLAP, FOLH1, Gi24, GITR, GITRL, GPR5, GP100, GPRC5D, HER2, HER3, ICOSL, ICOS, HHLA2, HMGB1, HVEM, IDO, IFNa, IgE, IGF1R, IL2Rbeta, IL1, ILIA, IL1B, IL1F10, IL2, IL4, IL4Ra, IL5, IL5R, IL6, IL7, IL7Ra, IL8, IL9, IL9R, IL10, rhILlO, IL12, IL13, IL13Ra1, IL13Ra2, IL15, IL17, IL17Rb, IL18, IL22, IL23, IL25, IL7, IL33, IL35, ITGB4, ITK, KIR, LAG3, LAMP1, leptin, LPFS2, MHC class II, MUC-1, MUC-16, NCR3LG1, NKG2D, NKp46, NTPDase-1, OX40, OX40L, PD-1, PD-L1, PD-L2, PROM1, 5152, SIRPalpha, SISP1, SLC, SPG64, ST2, STEAP1, STEAP2, Syk kinase, STEAP1, TROP2, TACI, TDO, TGFBETA, T14, TIGIT, TIM3, TLR, TLR2, TLR4, TLR5, TLR9, TMEF1, TNFa, TNFRSF7, Tp55, TREM1, TSLP, TSLPR, TWEAK, VEGF, VISTA, Vstm3, or WUCAM. In some aspects, VL2 and VH2 specifically bind to A2AR, APRIL, ATPDase, BAFF, BAFFR, BCMA, BlyS, BTK, BTLA, B7DC, B7H1, B7H2, B7H3, B7H4, B7H5, B7H6, B7H7, B7RP1, B7-4, C3, C5, CCL2, CCL3, CCL4, CCL5, CCL7, CCL8, CCL11, CCL15, CCL17, CCL19, CCL20, CCL21, CCL25, CCR3, CCR4, CD16A, CD19, CD20, CD24, CD27, CD28, CD30, CD38, CD39, CD40, CD40L, CD47, CD52, CD70, CD80, CD86, CD123, CD133, CD137, CD137L, CD160, CD272, CEACAM5, CLEC9, CLEC91, CRTH2, CSF-1, CSF-2, CSF-3, CXCL1, CXCL2, CXCL4, CXCL12, CXCL13, CXCR3, cMet, CTLA4, DLL3, DLL4, DNGR-1, E-cadherin, EGFR, ENTPD1, EpCAM, FCER1, FCER1A, FCER2, FGFR, FLAP, FOLH1, Gi24, GITR, GITRL, GPR5, GP100, GPRC5D, HER2, HER3, ICOSL, ICOS, HHLA2, HMGB1, HVEM, IDO, IFNa, IgE, IGF1R, IL2Rbeta, IL1, ILIA, IL1B, IL1F10, IL2, IL4, IL4Ra, IL5, IL5R, IL6, IL7, IL7Ra, IL8, IL9, IL9R, IL10, rhILlO, IL12, IL13, IL13Ra1, IL13Ra2, IL15, IL17, IL17Rb, IL18, IL22, IL23, IL25, IL7, IL33, IL35, ITGB4, ITK, KIR, LAG3, LAMP1, leptin, LPFS2, MHC class II, MUC-1, MUC-16, NCR3LG1, NKG2D, NKp46, NTPDase-1, OX40, OX40L, PD-1, PD-L1, PD-L2, PROM1, 5152, SIRPalpha, SISP1, SLC, SPG64, ST2, STEAP1, STEAP2, Syk kinase, STEAP1, TROP2, TACI, TDO, TGFBETA, T14, TIGIT, TIM3, TLR, TLR2, TLR4, TLR5, TLR9, TMEF1, TNFa, TNFRSF7, Tp55, TREM1, TSLP, TSLPR, TWEAK, VEGF, VISTA, Vstm3, or WUCAM. The VL1, VL2, VH1, and/or VH2 may specifically bind to CD28, cMet, Trop2, CD20, or CD19; VL3 and/or VH3 may specifically bind to CD28; and VL4 and/or VH4 may specifically bind to CD3. For example, the antigen binding polypeptide complex may specifically bind CD28, CD3, cMet, and Trop2. For example, the antigen binding polypeptide complex may specifically bind CD28, CD3, CD19, and CD20. For example, VL1 and VH1 may specifically bind to cMet. For example, VL2 and VH2 may specifically bind to cMet. For example, VL1 and VH1 may specifically bind to Trop2. For example, VL2 and VH2 may specifically bind to Trop2. For example, VL1 and VH1 may specifically bind to CD19. For example, VL2 and VH2 may specifically bind to CD19. For example, VL1 and VH1 may specifically bind to CD20. For example, VL2 and VH2 may specifically bind to CD20.

In some aspects of the antigen binding polypeptide complex described herein, VL4 and VH4 specifically bind to CD3, VL3 and VH3 specifically bind to CD28, VL2 and VH2 specifically bind to Trop2, and VL1 and VH1 specifically bind to cMet. In some aspects, VL4 and VH4 specifically bind to CD3, VL3 and VH3 specifically bind to CD28, VL2 and VH2 specifically bind to cMet, and VL1 and VH1 specifically bind to Trop2. In some aspects, VL4 and VH4 specifically bind to CD3, VL3 and VH3 specifically bind to Trop2, VL2 and VH2 specifically bind to CD28, and VL1 and VH1 specifically bind to cMet. In some aspects, VL4 and VH4 specifically bind to CD3, VL3 and VH3 specifically bind to Trop2, VL2 and VH2 specifically bind to cMet, and VL1 and VH1 specifically bind to CD28. In some aspects, VL4 and VH4 specifically bind to CD3, VL3 and VH3 specifically bind to cMet, VL2 and VH2 specifically bind to Trop2, and VL1 and VH1 specifically bind to CD28. In some aspects, VL4 and VH4 specifically bind to CD3, VL3 and VH3 specifically bind to cMet, VL2 and VH2 specifically bind to Trop2, and VL1 and VH1 specifically bind to CD28. In some aspects, VL4 and VH4 specifically bind to CD3, VL3 and VH3 specifically bind to CD28, VL2 and VH2 specifically bind to CD20, and VL1 and VH1 specifically bind to CD19. In some aspects, VL4 and VH4 specifically bind to CD3, VL3 and VH3 specifically bind to CD28, VL2 and VH2 specifically bind to CD19, and VL1 and VH1 specifically bind to CD20. In some aspects, VL4 and VH4 specifically bind to CD3, VL3 and VH3 specifically bind to CD20, VL2 and VH2 specifically bind to CD28, and VL1 and VH1 specifically bind to CD19. In some aspects, VL4 and VH4 specifically bind to CD3, VL3 and VH3 specifically bind to CD20, VL2 and VH2 specifically bind to CD19, and VL1 and VH1 specifically bind to CD28. In some aspects, VL4 and VH4 specifically bind to CD3, VL3 and VH3 specifically bind to CD19, VL2 and VH2 specifically bind to CD20, and VL1 and VH1 specifically bind to CD28. In some aspects, VL4 and VH4 specifically bind to CD3, VL3 and VH3 specifically bind to CD19, VL2 and VH2 specifically bind to CD28, and VL1 and VH1 specifically bind to CD20.

In some aspects of the antigen binding polypeptide complex described herein, VL4 and VH4 specifically bind to CD3, VL3 and VH3 specifically bind to CD28, VL2 and VH2 specifically bind to Trop2, and VL1 and VH1 specifically bind to cMet. In some aspects, VL4 and VH4 specifically bind to CD3, VL3 and VH3 specifically bind to CD28, VL2 and VH2 specifically bind to cMet, and VL1 and VH1 specifically bind to Trop2. In some aspects, VL4 and VH4 specifically bind to CD3, VL3 and VH3 specifically bind to Trop2, VL2 and VH2 specifically bind to CD28, and VL1 and VH1 specifically bind to cMet. In some aspects, VL4 and VH4 specifically bind to CD3, VL3 and VH3 specifically bind to Trop2, VL2 and VH2 specifically bind to cMet, and VL1 and VH1 specifically bind to CD28. In some aspects, VL4 and VH4 specifically bind to CD3, VL3 and VH3 specifically bind to cMet, VL2 and VH2 specifically bind to Trop2, and VL1 and VH1 specifically bind to CD28. In some aspects, VL4 and VH4 specifically bind to CD3, VL3 and VH3 specifically bind to cMet, VL2 and VH2 specifically bind to Trop2, and VL1 and VH1 specifically bind to CD28. In some aspects, VL4 and VH4 specifically bind to CD3, VL3 and VH3 specifically bind to CD28, VL2 and VH2 specifically bind to CD20, and VL1 and VH1 specifically bind to CD19. In some aspects, VL4 and VH4 specifically bind to CD3, VL3 and VH3 specifically bind to CD28, VL2 and VH2 specifically bind to CD19, and VL1 and VH1 specifically bind to CD20. In some aspects, VL4 and VH4 specifically bind to CD3, VL3 and VH3 specifically bind to CD20, VL2 and VH2 specifically bind to CD28, and VL1 and VH1 specifically bind to CD19. In some aspects, VL4 and VH4 specifically bind to CD3, VL3 and VH3 specifically bind to CD20, VL2 and VH2 specifically bind to CD19, and VL1 and VH1 specifically bind to CD28. In some aspects, VL4 and VH4 specifically bind to CD3, VL3 and VH3 specifically bind to CD19, VL2 and VH2 specifically bind to CD20, and VL1 and VH1 specifically bind to CD28. In some aspects, VL4 and VH4 specifically bind to CD3, VL3 and VH3 specifically bind to CD19, VL2 and VH2 specifically bind to CD28, and VL1 and VH1 specifically bind to CD20.

In some aspects, an antigen binding polypeptide complex comprises a first polypeptide, a second polypeptide, and a third polypeptide; wherein the first polypeptide has a structure represented by VL1-L1-CL; VL1-L1-CH1; VH1-L1-CL; or VH1-L1-CH1; wherein the second polypeptide has a structure represented by VH1-L2-CH1-L3-Fc; VH1-L2-CL-L3-Fc; VL1-L2-CH1-L3-Fc; or VL1-L2-CL-L3-Fc; wherein the third polypeptide has a structure represented by VL2-L4-VL3-L5-VH3-L6-VH2-L7-Fc; VL2-L4-VH3-L5-VL3-L6-VH2-L7-Fc; VH2-L4-VL3-L5-VH3-L6-VL2-L7-Fc; or VH2-L4-VH3-L5-VL3-L6-VL2-L7-Fc; wherein VL1 is a first immunoglobulin light chain variable region that specifically binds to A2AR, APRIL, ATPDase, BAFF, BAFFR, BCMA, BlyS, BTK, BTLA, B7DC, B7H1, B7H2, B7H3, B7H4, B7H5, B7H6, B7H7, B7RP1, B7-4, C3, C5, CCL2, CCL3, CCL4, CCL5, CCL7, CCL8, CCL11, CCL15, CCL17, CCL19, CCL20, CCL21, CCL25, CCR3, CCR4, CD16A, CD19, CD20, CD24, CD27, CD28, CD30, CD38, CD39, CD40, CD40L, CD47, CD52, CD70, CD80, CD86, CD123, CD133, CD137, CD137L, CD160, CD272, CEACAM5, CLEC9, CLEC91, CRTH2, CSF-1, CSF-2, CSF-3, CXCL1, CXCL2, CXCL4, CXCL12, CXCL13, CXCR3, cMet, CTLA4, DLL3, DLL4, DNGR-1, E-cadherin, EGFR, ENTPD1, EpCAM, FCER1, FCER1A, FCER2, FGFR, FLAP, FOLH1, Gi24, GITR, GITRL, GPR5, GP100, GPRC5D, HER2, HER3, ICOSL, ICOS, HHLA2, HMGB1, HVEM, IDO, IFNa, IgE, IGF1R, IL2Rbeta, IL1, ILIA, IL1B, IL1F10, IL2, IL4, IL4Ra, IL5, IL5R, IL6, IL7, IL7Ra, IL8, IL9, IL9R, IL10, rhILlO, IL12, IL13, IL13Ra1, IL13Ra2, IL15, IL17, IL17Rb, IL18, IL22, IL23, IL25, IL7, IL33, IL35, ITGB4, ITK, KIR, LAG3, LAMP1, leptin, LPFS2, MHC class II, MUC-1, MUC-16, NCR3LG1, NKG2D, NKp46, NTPDase-1, OX40, OX40L, PD-1, PD-L1, PD-L2, PROM1, S152, SIRPalpha, SISP1, SLC, SPG64, ST2, STEAP1, STEAP2, Syk kinase, STEAP1, TROP2, TACI, TDO, TGFBETA, T14, TIGIT, TIM3, TLR, TLR2, TLR4, TLR5, TLR9, TMEF1, TNFa, TNFRSF7, Tp55, TREM1, TSLP, TSLPR, TWEAK, VEGF, VISTA, Vstm3, or WUCAM; VL2 is a second immunoglobulin light chain variable region that specifically binds to CD3; VL3 is a third immunoglobulin light chain variable region that specifically binds to A2AR, APRIL, ATPDase, BAFF, BAFFR, BCMA, BlyS, BTK, BTLA, B7DC, B7H1, B7H2, B7H3, B7H4, B7H5, B7H6, B7H7, B7RP1, B7-4, C3, C5, CCL2, CCL3, CCL4, CCL5, CCL7, CCL8, CCL11, CCL15, CCL17, CCL19, CCL20, CCL21, CCL25, CCR3, CCR4, CD16A, CD19, CD20, CD24, CD27, CD28, CD30, CD38, CD39, CD40, CD40L, CD47, CD52, CD70, CD80, CD86, CD123, CD133, CD137, CD137L, CD160, CD272, CEACAM5, CLEC9, CLEC91, CRTH2, CSF-1, CSF-2, CSF-3, CXCL1, CXCL2, CXCL4, CXCL12, CXCL13, CXCR3, cMet, CTLA4, DLL3, DLL4, DNGR-1, E-cadherin, EGFR, ENTPD1, EpCAM, FCER1, FCER1A, FCER2, FGFR, FLAP, FOLH1, Gi24, GITR, GITRL, GPR5, GP100, GPRC5D, HER2, HER3, ICOSL, ICOS, HHLA2, HMGB1, HVEM, IDO, IFNa, IgE, IGF1R, IL2Rbeta, IL1, ILIA, IL1B, IL1F10, IL2, IL4, IL4Ra, IL5, IL5R, IL6, IL7, IL7Ra, IL8, IL9, IL9R, IL10, rhILlO, IL12, IL13, IL13Ra1, IL13Ra2, IL15, IL17, IL17Rb, IL18, IL22, IL23, IL25, IL7, IL33, IL35, ITGB4, ITK, KIR, LAG3, LAMP1, leptin, LPFS2, MHC class II, MUC-1, MUC-16, NCR3LG1, NKG2D, NKp46, NTPDase-1, OX40, OX40L, PD-1, PD-L1, PD-L2, PROM1, 5152, SIRPalpha, SISP1, SLC, SPG64, ST2, STEAP1, STEAP2, Syk kinase, STEAP1, TROP2, TACI, TDO, TGFBETA, T14, TIGIT, TIM3, TLR, TLR2, TLR4, TLR5, TLR9, TMEF1, TNFa, TNFRSF7, Tp55, TREM1, TSLP, TSLPR, TWEAK, VEGF, VISTA, Vstm3, or WUCAM; VH1 is a first immunoglobulin heavy chain variable region that specifically binds to A2AR, APRIL, ATPDase, BAFF, BAFFR, BCMA, BlyS, BTK, BTLA, B7DC, B7H1, B7H2, B7H3, B7H4, B7H5, B7H6, B7H7, B7RP1, B7-4, C3, C5, CCL2, CCL3, CCL4, CCL5, CCL7, CCL8, CCL11, CCL15, CCL17, CCL19, CCL20, CCL21, CCL25, CCR3, CCR4, CD16A, CD19, CD20, CD24, CD27, CD28, CD30, CD38, CD39, CD40, CD40L, CD47, CD52, CD70, CD80, CD86, CD123, CD133, CD137, CD137L, CD160, CD272, CEACAM5, CLEC9, CLEC91, CRTH2, CSF-1, CSF-2, CSF-3, CXCL1, CXCL2, CXCL4, CXCL12, CXCL13, CXCR3, cMet, CTLA4, DLL3, DLL4, DNGR-1, E-cadherin, EGFR, ENTPD1, EpCAM, FCER1, FCER1A, FCER2, FGFR, FLAP, FOLH1, Gi24, GITR, GITRL, GPR5, GP100, GPRC5D, HER2, HER3, ICOSL, ICOS, HHLA2, HMGB1, HVEM, IDO, IFNa, IgE, IGF1R, IL2Rbeta, IL1, ILIA, IL1B, IL1F10, IL2, IL4, IL4Ra, IL5, IL5R, IL6, IL7, IL7Ra, IL8, IL9, IL9R, IL10, rhILlO, IL12, IL13, IL13Ra1, IL13Ra2, IL15, IL17, IL17Rb, IL18, IL22, IL23, IL25, IL7, IL33, IL35, ITGB4, ITK, KIR, LAG3, LAMP1, leptin, LPFS2, MHC class II, MUC-1, MUC-16, NCR3LG1, NKG2D, NKp46, NTPDase-1, OX40, OX40L, PD-1, PD-L1, PD-L2, PROM1, S152, SIRPalpha, SISP1, SLC, SPG64, ST2, STEAP1, STEAP2, Syk kinase, STEAP1, TROP2, TACI, TDO, TGFBETA, T14, TIGIT, TIM3, TLR, TLR2, TLR4, TLR5, TLR9, TMEF1, TNFa, TNFRSF7, Tp55, TREM1, TSLP, TSLPR, TWEAK, VEGF, VISTA, Vstm3, or WUCAM; VH2 is a second immunoglobulin heavy chain variable region that specifically binds to CD3; VH3 is a third immunoglobulin heavy chain variable region that specifically binds to A2AR, APRIL, ATPDase, BAFF, BAFFR, BCMA, BlyS, BTK, BTLA, B7DC, B7H1, B7H2, B7H3, B7H4, B7H5, B7H6, B7H7, B7RP1, B7-4, C3, C5, CCL2, CCL3, CCL4, CCL5, CCL7, CCL8, CCL11, CCL15, CCL17, CCL19, CCL20, CCL21, CCL25, CCR3, CCR4, CD16A, CD19, CD20, CD24, CD27, CD28, CD30, CD38, CD39, CD40, CD40L, CD47, CD52, CD70, CD80, CD86, CD123, CD133, CD137, CD137L, CD160, CD272, CEACAM5, CLEC9, CLEC91, CRTH2, CSF-1, CSF-2, CSF-3, CXCL1, CXCL2, CXCL4, CXCL12, CXCL13, CXCR3, cMet, CTLA4, DLL3, DLL4, DNGR-1, E-cadherin, EGFR, ENTPD1, EpCAM, FCER1, FCER1A, FCER2, FGFR, FLAP, FOLH1, Gi24, GITR, GITRL, GPR5, GP100, GPRC5D, HER2, HER3, ICOSL, ICOS, HHLA2, HMGB1, HVEM, IDO, IFNa, IgE, IGF1R, IL2Rbeta, IL1, ILIA, IL1B, IL1F10, IL2, IL4, IL4Ra, IL5, IL5R, IL6, IL7, IL7Ra, IL8, IL9, IL9R, IL10, rhILlO, IL12, IL13, IL13Ra1, IL13Ra2, IL15, IL17, IL17Rb, IL18, IL22, IL23, IL25, IL7, IL33, IL35, ITGB4, ITK, KIR, LAG3, LAMP1, leptin, LPFS2, MHC class II, MUC-1, MUC-16, NCR3LG1, NKG2D, NKp46, NTPDase-1, OX40, OX40L, PD-1, PD-L1, PD-L2, PROM1, S152, SIRPalpha, SISP1, SLC, SPG64, ST2, STEAP1, STEAP2, Syk kinase, STEAP1, TROP2, TACI, TDO, TGFBETA, T14, TIGIT, TIM3, TLR, TLR2, TLR4, TLR5, TLR9, TMEF1, TNFa, TNFRSF7, Tp55, TREM1, TSLP, TSLPR, TWEAK, VEGF, VISTA, Vstm3, or WUCAM; Fc is a region comprising an immunoglobulin heavy chain constant region 2 (CH2), an immunoglobulin heavy chain constant region 3 (CH3), and optionally, an immunoglobulin hinge; and L1-L7 are amino acid linkers. For example, the antigen binding polypeptide complex may specifically bind one or more of CD20, CD3 and CD28. For example, the antigen binding polypeptide complex may specifically bind CD20 and CD3. For example, the antigen binding polypeptide complex may specifically bind to CD20, CD3 and CD28. For example, VH1 and VL1 may specifically bind to CD20. For example, VH2 and VL2 may specifically bind to CD3. For example, VH2 and VL2 may specifically bind to CD28. For example, VH3 and VL3 may specifically bind to CD28. For example, VH3 and VL3 may specifically bind to CD20. For example, VH3 and VL3 may specifically bind to CD3. For example, VH1 and VL1 may specificially bind to CD20, VH2 and VL2 may specifically bind to CD3, and VH3 and VL3 may specifically bind to CD20. For example, VH1 and VL1 may specificially bind to CD20, VH2 and VL2 may specifically bind to CD3, and VH3 and VL3 may specifically bind to CD28. For example, VH1 and VL1 may specificially bind to CD20, VH2 and VL2 may specifically bind to CD28, and VH3 and VL3 may specifically bind to CD3.

For the avoidance of doubt, all the antigen binding polypeptide complex structures described herein can be combined with any one or more of the targets described herein. Any and all disclosure herein in relation to targets for antigen binding polypeptide complexs of the invention is generally applicable, and applies equally and without reservation to each and every antigen binding polypeptide complex described herein. For the avoidance of doubt, the VL1, VL2, VL3, VL4, VH1, VH2, VH3, and/or VH4 of each and every antigen binding polypeptide complex described herein may independently bind to any one of said particularly preferred targets.

In some aspects, an antigen binding polypeptide complex (e.g., an antibody or antigen binding fragment thereof) comprises an immunoglobulin hinge. In some aspects, the immunoglobulin hinge comprises an upper hinge region, a middle hinge region, a lower hinge region, or a combination thereof.

As used herein, an antigen binding polypeptide complex (e.g., an antibody or antigen binding fragment thereof), or region or domain thereof that “specifically binds” refers to its association with an epitope by its antigen binding domain, and that the binding entails some complementarity between the antigen binding domain and the epitope. Specific binding to an epitope occurs where there is binding to that epitope via its antigen binding domain more readily than there would be binding to a random, unrelated epitope.

As used herein, an “epitope” refers to a localized region of an antigen to which an antigen binding polypeptide complex (e.g., antibody or antigen binding fragment thereof) can specifically bind. An epitope can be, for example, contiguous amino acids of a polypeptide (linear or contiguous epitope) or an epitope can, for example, come together from two or more non-contiguous regions of a polypeptide or polypeptides (conformational, non-linear, discontinuous, or non-contiguous epitope). In some aspects, the epitope to which an antibody or antigen-binding fragment thereof binds can be determined by, e.g., NMR spectroscopy, X-ray diffraction crystallography studies, ELISA assays, hydrogen/deuterium exchange coupled with mass spectrometry (e.g., liquid chromatography electrospray mass spectrometry), array-based oligo-peptide scanning assays, and/or mutagenesis mapping (e.g., site-directed mutagenesis mapping). See, e.g., Giege R et al., (1994) Acta Crystallogr D Biol Crystallogr 50(Pt 4): 339-350; McPherson A (1990) Eur J Biochem 189: 1-23; Chayen N E (1997) Structure 5: 1269-1274; McPherson A (1976) J Biol Chem 251: 6300-6303; Meth Enzymol (1985) volumes 114 & 115, eds Wyckoff H W et al., U.S. Pub. No. 2004/0014194), Bricogne G (1993) Acta Crystallogr D Biol Crystallogr 49(Pt 1): 37-60, Bricogne G (1997) Meth Enzymol 276A: 361-423, ed Carter C W, and Roversi et al., (2000) Acta Crystallogr D Biol Crystallogr 56(Pt 10): 1316-1323 (X-ray diffraction crystallography studies); and Champe et al., (1995) J Biol Chem 270: 1388-1394 and Cunningham B C & Wells J A (1989) Science 244: 1081-1085 (mutagenesis mapping).

Specific binding can be represented by a “binding affinity.” Binding affinity refers to an intrinsic binding affinity which reflects a 1:1 interaction between members of a binding pair (e.g., an antigen binding polypeptide complex and an antigen). Binding affinity can be measured and/or expressed in several ways known in the art, including, but not limited to, equilibrium dissociation constant (K_{D}). K_{D} is calculated from the quotient of k_{off}/kₒₙ, where kₒₙ refers to the association rate constant of, e.g., an antigen binding polypeptide complex to an antigen, and k_{off} refers to the dissociation of, e.g., an antigen binding polypeptide complex from an antigen. The kₒₙ and k_{off} can be determined by techniques known to one of ordinary skill in the art, such as Octet BLI, BIAcore® or KinExA.

Accordingly, in some aspects, an antigen binding polypeptide complex of the invention is an antibody or antigen binding fragment thereof.

In some aspects, the antibody or antigen binding fragment thereof specifically binds to an antigen with an equilibrium dissociation constant (K_{D}) of from about 10 μM to about 1 pM. In some aspects, the antibody is IgG, IgM, IgE, IgA or IgD. For example, the antibody may be IgG. For example, the antibody may be IgM. For example, the antibody may be IgE. For example, the antibody may be IgA. For example, the antibody may be IgD. In some aspects, the IgG is IgG1, IgG2, IgG3 or IgG4. For example, the antibody may be IgG1. For example, the antibody may be IgG2. For example, the antibody may be IgG3. For example, the antibody may be IgG4. In some aspects, the antigen binding fragment is a Fab, scFab, Fab′, F(ab′)2, Fv or scFv. For example, the antigen binding fragment may be a Fab. For example, the antigen binding fragment may be a scFab. For example, the antigen binding fragment may be a Fab′. For example, the antigen binding fragment may be a F(ab′)2. For example, the antigen binding fragment may be a Fv. For example, the antigen binding fragment may be a scFv. In some aspects, the antibody is human or humanized. For example, the antibody may be human. For the example, the antibody may be humanized.

In some aspects, an antigen binding polypeptide complex of the invention (e.g., an antibody or antigen binding fragment thereof) is bivalent, trivalent, tetravalent, pentavalent or hexavalent.

### Amino Acid Linkers

In some aspects, an antigen binding polypeptide complex (e.g., an antibody or antigen binding fragment thereof) of the invention comprises one or more amino acid linkers between one or more regions of the antigen binding polypeptide complex.

As used herein, an “amino acid linker” refers to a single amino acid or short amino acid sequence that is capable of joining two polypeptide regions of the invention described herein in a stable manner that maintains or promotes a function associated with the polypeptide regions. In some aspects, an amino acid linker is represented herein in a structure of an antigen binding polypeptide complex by the abbreviation “1” or “L” and a number (e.g., L1 to denote a first linker, L2 to denote a second linker, L3 to denote a third linker, L4 to denote a fourth linker, L5 to denote a fifth linker, L6 to denote a sixth linker, L7 to denote a seventh linker, L8 to denote an eighth linker, and so on). In some aspects, such enumerated amino acid linkers (e.g., L1) can have the same or different sequence as any other enumerated amino acid linker (e.g., L2, etc.). Furthermore, in some aspects, an enumerated amino acid linker present in one polypeptide (e.g., L1 on a first polypeptide of an antigen binding polypeptide complex structure described herein) can have the same or different sequence as the same enumerated amino acid linker present in another polypeptide (e.g., L1 on a second polypeptide, third polypeptide, etc. of an antigen binding polypeptide complex structure described herein).

In some aspects, an amino acid linker has a length of from 0 amino acids (i.e., an amino acid linker is not present) to about 50 amino acids (e.g., one or more of L1, L2, L3, L4, L5, L6, L7, L8, L9, L10, L11, L12, L13, L14, L15, L16, L17, L18, L19, L20, etc. of a first, second, third, fourth, etc. polypeptide of an antigen binding polypeptide complex structure described herein). In some aspects, the amino acid linker has a length of from 0 amino acids to about 45 amino acids, 0 amino acids to about 40 amino acids, 0 amino acids to about 35 amino acids, 0 amino acids to about 30 amino acids, 0 amino acids to about 25 amino acids, 0 amino acids to about 20 amino acids, 0 amino acids to about 15 amino acids, 0 amino acids to about 10 amino acids, 0 amino acids to about 5 amino acids, about 1 amino acid to about 45 amino acids, about 1 amino acid to about 40 amino acids, about 1 amino acid to about 35 amino acids, about 1 amino acid to about 30 amino acids, about 1 amino acid to about 25 amino acids, about 1 amino acid to about 20 amino acids, 1 amino acid to about 15 amino acids, about 1 amino acid to about 10 amino acids, about 1 amino acid to about 5 amino acids, about 5 amino acids to about 50 amino acids, about 5 amino acids to about 45 amino acids, about 5 amino acids to about 40 amino acids, about 5 amino acids to about 35 amino acids, about 5 amino acids to about 30 amino acids, about 5 amino acids to about 25 amino acids, about 5 amino acids to about 20 amino acids, about 5 amino acids to about 15 amino acids, about 5 amino acids to about 10 amino acids, about 10 amino acids to about 50 amino acids, about 10 amino acids to about 45 amino acids, about 10 amino acids to about 40 amino acids, about 10 amino acids to about 35 amino acids, about 10 amino acids to about 30 amino acids, about 10 amino acids to about 25 amino acids, about 10 amino acids to about 20 amino acids, about 10 amino acids to about 15 amino acids, about 15 amino acids to about 50 amino acids, about 15 amino acids to about 45 amino acids, about 15 amino acids to about 40 amino acids, about 15 amino acids to about 35 amino acids, about 15 amino acids to about 30 amino acids, about 15 amino acids to about 25 amino acids, about 15 amino acids to about 20 amino acids, about 20 amino acids to about 50 amino acids, about 20 amino acids to about 45 amino acids, about 20 amino acids to about 40 amino acids, about 20 amino acids to about 35 amino acids, about 20 amino acids to about 30 amino acids, about 20 amino acids to about 25 amino acids, about 25 amino acids to about 50 amino acids, about 25 amino acids to about 45 amino acids, about 25 amino acids to about 40 amino acids, about 25 amino acids to about 35 amino acids, about 25 amino acids to about 30 amino acids, about 30 amino acids to about 50 amino acids, about 30 amino acids to about 45 amino acids, about 30 amino acids to about 40 amino acids, about 30 amino acids to about 35 amino acids, about 40 amino acids to about 50 amino acids, about 40 amino acids to about 45 amino acids, or about 45 amino acids to about 50 amino acids.

In some aspects, the amino acid linker has 0 amino acids (i.e., an amino acid linker is not present) or about 1, about 2, about 3, about 4, about 5, about 6, about 7, about 8, about 9, about 10, about 11, about 12, about 13, about 14, about 15, about 16, about 17, about 18, about 19, about 20, about 25, about 30, about 35, about 40, about 45, or about 50 amino acids (e.g., one or more of L1, L2, L3, L4, L5, L6, L7, L8, L9, L10, L11, L12, L13, L14, L15, L16, L17, L18, L19, L20, etc. of a first, second, third, fourth, etc. polypeptide of an antigen binding polypeptide complex structure described herein).

In some aspects, the amino acid linker consists of one or more amino acid residues (e.g., one or more of L1, L2, L3, L4, L5, L6, L7, L8, L9, L10, L11, L12, L13, L14, L15, L16, L17, L18, L19, L20, etc. of a first, second, third, fourth, etc. polypeptide of an antigen binding polypeptide complex structure described herein). In some aspects, the amino acid residues are selected from the group consisting of glycine, alanine, serine, threonine, cysteine, asparagine, glutamine, leucine, isoleucine, valine, proline, histidine, aspartic acid, glutamic acid, lysine, arginine, methionine, phenylalanine, tryptophan, and tyrosine.

In some aspects, an amino acid linker of the invention is non-immunogenic. In some aspects, the non-immunogenic linker consists of serine, glycine and/or alanine residues, or consists of serine and/or glycine residues. In some aspects, an amino acid linker of the invention does not contain a T cell epitope or consensus T cell epitope.

In some aspects, the amino acid linker consists of one or more residues of alanine, cysteine, glycine, isoleucine, leucine, methionine, phenylalanine, proline, tryptophan, tyrosine, valine (e.g., one or more of L1, L2, L3, L4, L5, L6, L7, L8, L9, L10, L11, L12, L13, L14, L15, L16, L17, L18, L19, L20, etc. of a first, second, third, fourth, etc. polypeptide of an antigen binding polypeptide complex structure described herein).

Amino acid linker sequences that can be used with the antigen binding polypeptide complexes (e.g., an antibody or antigen binding fragment thereof) of the invention are well known and can be incorporated into antigen binding polypeptide complexes of the invention using routine molecular biology and recombinant DNA techniques. See, e.g., Chen et al., Adv Drug Deliv Rev., 65(10):1357-1369, 2013; and Chichili et al., Protein Sci., 22(2):153-167, 2013.

In some aspects, the amino acid linker (e.g., one or more of L1, L2, L3, L4, L5, L6, L7, L8, L9, L10, L11, L12, L13, L14, L15, L16, L17, L18, L19, L20, etc. of a first, second, third, fourth, etc. polypeptide of an antigen binding polypeptide complex structure described herein) has the sequence of g, a, gss, asg, ggssg, ggsgs, gssgs, gtvaa, asggs, astgg, asggsg, ggsgssg, ggsggssgss, sggsgssggs, ggsggsgsgggsasgsg, ggsggsgsggggsasgsg, gggssggggsggsgsggsgs, ggggsggsgsggggsasgsg, gggssggsgsggsgsggsgs, sggssggsgsggsgsggsgssg, gsgssggggsggsgsggsgssg, ggggsgsggsgggssggggsggggsggggsggggsggggs, ggggsggggsggggsggggsggggsggggsggggsggggs, ggggsgsggsgggssggggsggggsggggsggggsggggssss, or ggggsgsggsgggssggggsggggsggggsggggsggggssssgs (SEQ ID NOs:1-21), the sequence of any one of SEQ ID NOs:444, 567, 576 (GGS) and 607, or a sequence having at least 50%, at least 60%, at least 70%, at least 80%, at least 90%, at least 91%, at least 92%, at least 93%, at least 94%, at least 95%, at least 96%, at least 97%, at least 98%, at least 99% or 100% identity to any one of SEQ ID NOs:1-21 and SEQ ID NOs:444, 567, 576 (GGS) and 607.

In some aspects, an amino acid linker of an antigen binding polypeptide complex described herein (e.g., an antibody or an antibody or antigen binding fragment thereof) is a cleavable linker. As used herein a “cleavable linker” is an amino acid linker that can connect two or more polypeptides together and then can be cleaved once exposed to, for example, an enzyme, photo-irradiation, or chemical reagent. In some aspects, the enzyme is present at the site of a tumor.

In some aspects, the cleavable linker is cleaved by a matrix metalloproteinase (MMP). In some aspects, the cleavable linker is cleaved by a type II transmembrane serine protease.

In some aspects, the cleavable linker (e.g., one or more of L1, L2, L3, L4, L5, L6, L7, L8, L9, L10, L11, L12, L13, L14, L15, L16, L17, L18, L19, L20, etc. of a first, second, third, fourth, etc. polypeptide of an antigen binding polypeptide complex structure described herein) has the amino acid sequence of GPAALV, GSGRKG, GPLGLTG, GPSGLVG, GLVGRKAG, GPAGLVG, GPAGLVSG, STRKAGG, ASTRKAG, or ASTRKAGG (SEQ ID NOs:22-31), or a sequence having at least 50%, at least 60%, at least 70%, at least 80%, at least 90%, at least 91%, at least 92%, at least 93%, at least 94%, at least 95% at least 96%, at least 97%, at least 98%, at least 99% or 100% identity to any one of SEQ ID NOs:22-31.

In some aspects of an antigen binding polypeptide complex described herein, L5, L8, or L5 and L8 are cleavable linkers. In some aspects, VH3 and VL3 of the antigen binding polypeptide complex specifically bind to CD3. In some aspects, VH1, VL1, VH2 and VL2 of the antigen binding polypeptide complex specifically bind to a TAA. In some aspects, VH1 and VL1 specifically bind to a TAA, and VH2 and VL2 specifically bind to a TAA.

In some aspects of an antigen binding polypeptide complex described herein, L4, L7, or L4 and L7 are cleavable linkers. In some aspects, VH3 and VL3 of an antigen binding polypeptide complex described herein specifically bind to CD3. In some aspects, VH1, VL1, VH2 and VL2 of the antigen binding polypeptide complex specifically bind to a TAA. In some aspects, VH1 and VL1 of the antigen binding polypeptide complex specifically bind to a TAA, and VH2 and VL2 specifically bind to a TAA.

In some aspects of an antigen binding polypeptide complex described herein, L9, L12, or L9 and L12 are cleavable linkers. In some aspects, VH4 and VL4 of the antigen binding polypeptide complex specifically bind to CD3. In some aspects, VH1, VL1, VH2, VL2, VH3 and VL3 specifically bind to a TAA. In some aspects, VH1 and VL1 specifically bind to a TAA, VH2 and VL2 specifically bind to a TAA, and VH3 and VL3 specifically bind to CD28. In some aspects, VH1 and VL1 specifically bind to CD28, VH2 and VL2 specifically bind to a TAA, and VH3 and VL3 specifically bind to a TAA. In some aspects, VH1 and VL1 specifically bind to cMet, VH2 and VL2 specifically bind to Trop2, and VH3 and VL3 specifically bind to CD28. In some aspects, VH1 and VL1 specifically bind to Trop2, VH2 and VL2 specifically bind to cMet, and VH3 and VL3 specifically bind to CD28. In some aspects, VH1 and VL1 specifically bind to cMet, VH2 and VL2 specifically bind to CD28, and VH3 and VL3 specifically bind to Trop2. In some aspects, VH1 and VL1 specifically bind to CD28, VH2 and VL2 specifically bind to cMet, and VH3 and VL3 specifically bind to Trop2. In some aspects, VH1 and VL1 specifically bind to Trop2, VH2 and VL2 specifically bind to CD28, and VH3 and VL3 specifically bind to cMet. In some aspects, VH1 and VL1 specifically bind to CD28, VH2 and VL2 specifically bind to Trop2, and VH3 and VL3 specifically bind to cMet. In some aspects, VH1 and VL1 specifically bind to CD20, VH2 and VL2 specifically bind to CD19, and VH3 and VL3 specifically bind to CD28. In some aspects, VH1 and VL1 specifically bind to CD19, VH2 and VL2 specifically bind to CD20, and VH3 and VL3 specifically bind to CD28. In some aspects, VH1 and VL1 specifically bind to CD20, VH2 and VL2 specifically bind to CD28, and VH3 and VL3 specifically bind to CD19. In some aspects, VH1 and VL1 specifically bind to CD28, VH2 and VL2 specifically bind to CD20, and VH3 and VL3 specifically bind to CD19. In some aspects, VH1 and VL1 specifically bind to CD19, VH2 and VL2 specifically bind to CD28, and VH3 and VL3 specifically bind to CD20. In some aspects, VH1 and VL1 specifically bind to CD28, VH2 and VL2 specifically bind to CD19, and VH3 and VL3 specifically bind to CD20.

In some aspects of an antigen binding polypeptide complex described herein, L1, L3, or L1 and L3 are cleavable linkers, or L5, L7, or L5 and L7 are cleavable linkers. In some aspects of an antigen binding polypeptide complex described herein, L9, L11, or L9 and L11 are cleavable linkers, or L13, L15, or L13 and L15 are cleavable linkers. In some aspects, VH1 and VL1, VH2 and VL2, VH3 and VL3, or VH4 and VL4 of the antigen binding polypeptide complex specifically bind to CD3. In some aspects, one or more of VH1 and VL1, VH2 and VL2, VH3 and VL3, and VH4 and VL4 specifically bind to a TAA.

In some aspects, VH1 and VH2 of an antigen binding polypeptide complex described herein are joined by a cleavable linker. In some aspects, VH1 and VL2 of an antigen binding polypeptide complex described herein are joined by a cleavable linker. In some aspects, VL1 and VH2 of an antigen binding polypeptide complex described herein are joined by a cleavable linker. In some aspects, VL1 and VL2 of an antigen binding polypeptide complex described herein are joined by a cleavable linker. In some aspects, VH1 and VH2 of an antigen binding polypeptide complex described herein are joined by a noncleavable linker having the amino acid sequence of GS (GS linker). In some aspects, VH1 and VL2 of an antigen binding polypeptide complex described herein are joined by a GS linker. In some aspects, VL1 and VH2 of an antigen binding polypeptide complex described herein are joined by a GS linker. In some aspects, VL1 and VL2 of an antigen binding polypeptide complex described herein are joined by a GS linker.

In some aspects, VL2 and VL1 of an antigen binding polypeptide complex described herein are joined by a cleavable linker. In some aspects, VH2 and VL1 of an antigen binding polypeptide complex described herein are joined by a cleavable linker. In some aspects, VL2 and VH1 of an antigen binding polypeptide complex described herein are joined by a cleavable linker. In some aspects, VH2 and VH1 of an antigen binding polypeptide complex described herein are joined by a cleavable linker. In some aspects, VL2 and VL1 of an antigen binding polypeptide complex described herein are joined by a GS linker. In some aspects, VH2 and VL1 of an antigen binding polypeptide complex described herein are joined by a GS linker. In some aspects, VL2 and VH1 of an antigen binding polypeptide complex described herein are joined by a GS linker. In some aspects, VH2 and VH1 of an antigen binding polypeptide complex described herein are joined by a GS linker.

In some aspects, VH3 and VH4 of an antigen binding polypeptide complex described herein are joined by a cleavable linker. In some aspects, VH3 and VL4 of an antigen binding polypeptide complex described herein are joined by a cleavable linker. In some aspects, VL3 and VH4 of an antigen binding polypeptide complex described herein are joined by a cleavable linker. In some aspects, VL3 and VL4 of an antigen binding polypeptide complex described herein are joined by a cleavable linker. In some aspects, VH3 and VH4 of an antigen binding polypeptide complex described herein are joined by a GS linker. In some aspects, VH3 and VL4 of an antigen binding polypeptide complex described herein are joined by a GS linker. In some aspects, VL3 and VH4 of an antigen binding polypeptide complex described herein are joined by a GS linker. In some aspects, VL3 and VL4 of an antigen binding polypeptide complex described herein are joined by a GS linker.

In some aspects, VL4 and VL3 of an antigen binding polypeptide complex described herein are joined by a cleavable linker. In some aspects, VH4 and VL3 of an antigen binding polypeptide complex described herein are joined by a cleavable linker. In some aspects, VL4 and VH3 of an antigen binding polypeptide complex described herein are joined by a cleavable linker. In some aspects, VH4 and VH3 of an antigen binding polypeptide complex described herein are joined by a cleavable linker. In some aspects, VL4 and VL3 of an antigen binding polypeptide complex described herein are joined by a GS linker. In some aspects, VH4 and VL3 of an antigen binding polypeptide complex described herein are joined by a GS linker. In some aspects, VL4 and VH3 of an antigen binding polypeptide complex described herein are joined by a GS linker. In some aspects, VH4 and VH3 of an antigen binding polypeptide complex described herein are joined by a GS linker.

In some aspects, VH1 and VH2 of an antigen binding polypeptide complex described herein are joined by a cleavable linker, and VL2 and VL1 of an antigen binding polypeptide complex described herein are joined by a GS linker. In some aspects, VH1 and VL2 of an antigen binding polypeptide complex described herein are joined by a cleavable linker, and VH2 and VL1 of an antigen binding polypeptide complex described herein are joined by a GS linker. In some aspects, VL1 and VH2 of an antigen binding polypeptide complex described herein are joined by a cleavable linker, and VL2 and VH1 of an antigen binding polypeptide complex described herein are joined by a GS linker. In some aspects, VL1 and VL2 of an antigen binding polypeptide complex described herein are joined by a cleavable linker, and VH2 and VH1 of an antigen binding polypeptide complex described herein are joined by a GS linker. In some aspects, VH1 and VH2 of an antigen binding polypeptide complex described herein are joined by a GS linker, and VL2 and VL1 of an antigen binding polypeptide complex described herein are joined by a cleavable linker. In some aspects, VH1 and VL2 of an antigen binding polypeptide complex described herein are joined by a GS linker, and VH2 and VL1 of an antigen binding polypeptide complex described herein are joined by a cleavable linker. In some aspects, VL1 and VH2 of an antigen binding polypeptide complex described herein are joined by a GS linker, and VL2 and VH1 of an antigen binding polypeptide complex described herein are joined by a cleavable linker. In some aspects, VL1 and VL2 of an antigen binding polypeptide complex described herein are joined by a GS linker, and VH2 and VH1 of an antigen binding polypeptide complex described herein are joined by a cleavable linker.

In some aspects, VH3 and VH4 of an antigen binding polypeptide complex described herein are joined by a cleavable linker, and VL4 and VL3 of an antigen binding polypeptide complex described herein are joined by a GS linker. In some aspects, VH3 and VL4 of an antigen binding polypeptide complex described herein are joined by a cleavable linker, and VH4 and VL3 of an antigen binding polypeptide complex described herein are joined by a GS linker. In some aspects, VL3 and VH4 of an antigen binding polypeptide complex described herein are joined by a cleavable linker, and VL4 and VH3 of an antigen binding polypeptide complex described herein are joined by a GS linker. In some aspects, VL3 and VL4 of an antigen binding polypeptide complex described herein are joined by a cleavable linker, and VH4 and VH3 of an antigen binding polypeptide complex described herein are joined by a GS linker. In some aspects, VH3 and VH4 of an antigen binding polypeptide complex described herein are joined by a GS linker, and VL4 and VL3 of an antigen binding polypeptide complex described herein are joined by a cleavable linker. In some aspects, VH3 and VL4 of an antigen binding polypeptide complex described herein are joined by a GS linker, and VH4 and VL3 of an antigen binding polypeptide complex described herein are joined by a cleavable linker. In some aspects, VL3 and VH4 of an antigen binding polypeptide complex described herein are joined by a GS linker, and VL4 and VH3 of an antigen binding polypeptide complex described herein are joined by a cleavable linker. In some aspects, VL3 and VL4 of an antigen binding polypeptide complex described herein are joined by a GS linker, and VH4 and VH3 of an antigen binding polypeptide complex described herein are joined by a cleavable linker.

### Detectable Labels and Drug Conjugates

In some aspects, an antigen binding polypeptide complex (e.g., an antibody or antigen binding fragment thereof) of the invention comprises one or more detectable labels. An antigen binding polypeptide complex (e.g., an antibody or antigen binding fragment thereof) containing a detectable label is useful in therapeutic, diagnostic, imaging (e.g., radioimaging), or basic research applications.

In some aspects, the detectable label is a radioactive label. Examples of a radioactive label include, but are not limited to, the isotopes ³H, ¹⁴C, ³²P, ³⁵S, ³⁶Cl, ⁵¹Cr, ⁵⁷Co, ⁵⁸Co, ⁵⁹Fe, ⁹⁰Y, ¹²¹I, ¹²⁴I, ¹²⁵I, ¹³¹I, ¹¹¹In, ¹¹⁷Lu ²¹¹At, ¹⁹⁸Au, ⁶⁷Cu, ²²⁵Ac, ²¹³Bi, ⁹⁹Tc, ¹⁸⁶Re and ⁸⁹Zr.

In some aspects, the detectable label is a chemiluminescent label, fluorescent label, enzyme, biotin, or a combination thereof.

In some aspects, the detectable label is a peptide tag. In some aspects, the peptide tag is located at the N-terminus of the polypeptide or polypeptide complex. In some aspects, the peptide tag is located at the C-terminus of the polypeptide or polypeptide complex. In some aspects, the peptide tag is an affinity tag or fusion tag.

In some aspects, the detectable label is a polyhistidine tag, polyarginine tag, glutathione-S-transferase (GST), maltose binding protein (MBP), chitin binding protein (CBP), Strep-tag, thioredoxin (TRX), poly(NANP), FLAG tag, ALFA-tag, V5-tag, Myc-tag, hemagglutinin (HA) tag, Spot tag, T7 tag, NE tag, or green fluorescence protein (GFP), or a combination thereof. In some aspects, the polyhistidine tag consists of from about 4 to about 10 histidine residues. In some aspects, the polyhistidine tag consists of about 4, about 5, about 6, about 7, about 8, about 9, or about 10 histidine residues.

Additional examples of detectable labels and methods for introducing detectable labels into a polypeptide are known and include routine chemical, molecular biology and recombinant DNA techniques. See, e.g., Hnatowich et al., Science, 220(4597):613-615, 1983; Yao et al., Int. J. Mol. Sci., 17(2):194, 2016; Kimple et al., Curr. Protoc. Protein Sci., 73:Unit 9.9, 2013; Sambrook J, Fritsch E F. Molecular Cloning: A Laboratory Manual. Cold Spring Harbor Laboratory Press; Cold Spring Harbor, N.Y.: 1989; Molecular Cell Biology, 4^{th} edition, Section 3.5, Purifying, Detecting and Characterizing Proteins; and Mahmoodi et al., Cogent Biology, 5(1):DOI: 10/1080/23312025.2019.1665406.

In some aspects, an antigen binding polypeptide complex (e.g., an antibody or antigen binding fragment thereof) of the invention is conjugated to an agent as an antibody-drug conjugate (ADC). An ADC of the invention is useful in therapeutic, diagnostic, imaging (e.g., radioimaging), or basic research applications.

In some aspects, an antigen binding polypeptide complex (e.g., an antibody or antigen binding fragment thereof) of the invention is conjugated to a cytotoxic agent, immunomodulating agent, imaging agent, or therapeutic protein, typically via a linker. The linker can comprise a cleavable unit or can be non-cleavable. Cleavable units include, for example, disulfide containing linkers that are cleavable through disulfide exchange, acid-labile linkers that are cleavable at acidic pH, and linkers that are cleavable by hydrolases, esterases, peptidases, and glucoronidases (e.g., peptide linkers and glucoronide linkers). Non-cleavable linkers are believed to release drug via a proteolytic antibody degradation mechanism.

Methods for making an ADC are known and include, but are not limited to, conjugation via thiols, amides, aldehydes, or azides, as well as other routine chemical, molecular biology and recombinant DNA techniques. See, e.g., Yao et al., Int. J. Mol. Sci., 17(2):194, 2016; Sambrook J, Fritsch E F. Molecular Cloning: A Laboratory Manual. Cold Spring Harbor Laboratory Press; Cold Spring Harbor, N.Y.: 1989; Molecular Cell Biology, 4^{th} edition, Section 3.5, Purifying, Detecting and Characterizing Proteins; and Mahmoodi et al., Cogent Biology, 5(1):DOI: 10/1080/23312025.2019.1665406.

### Modifications

In some aspects, the invention is directed to an antigen binding polypeptide complex (e.g., an antibody or antigen binding fragment thereof) comprising an effector function mutation or half-life extension mutation.

Effector functions are an important part of the humoral immune response and form an link between innate and adaptive immunity. Most effector functions are induced via the Fc region of an antibody, which can interact with complement proteins and specialized Fc receptors. As used herein, an “effector function mutation” refers to a change in the amino acid sequence, typically in the Fc region, which increases or decreases effector function, for example, increasing binding affinity of Fc for specific Fc receptors, or increasing antibody-dependent cellular cytotoxicity (ADCC) activity.

“Half-life” of a pharmaceutically active substance is the time it takes for the amount of the substance, once administered to the body, to reduce by half. A “half-life extension mutation” of an antigen binding polypeptide complex of the invention refers to a change in the amino acid sequence, typically in the Fc region, which increases the half-life of the antigen binding polypeptide complex (e.g., by increasing Fc receptor binding affinity, slowing off-rate for Fc and Fc receptors, and/or increased sialylation).

Examples of effector function mutations that increase function include, but are not limited to, the following substitutions in the Fc region, based on the EU numbering scheme: S298A/E333A/K334A, S239D/I332E, S239D/A330L/I332E, and G236A/S239D/I332E. Examples of effector function mutations that decrease function include, but are not limited to, the following substitutions in the Fc region, based on the EU numbering scheme: N297A and L234A/L235A. Additional examples of effector function mutations, half-life extension mutations and methods for incorporating the same into an amino acid sequence are known and described, for example, in Saunders, “Conceptual Approaches to Modulating Antibody Effector Functions and Circulation Half-Life,” Front. Immunol. Jun. 7, 2019.

In some aspects, the invention is directed to an antigen binding polypeptide complex (e.g., an antibody or antigen binding fragment thereof) comprising one or more knob-into-hole modifications.

The term “knob-into-hole modification” as used herein, refers to a genetic modification that directs the pairing of two polypeptides to promote heterodimerization. In some aspects, the modification introduces a protuberance (knob) into one polypeptide and a cavity (hole) into the other polypeptide at an interface in which the two polypeptides interact. In some aspects, a knob-into-hole modification can be created by introducing only a hole modification, for example, by replacing an amino acid residue with a smaller side chain than the original amino acid residue (e.g., a substitution of one or more serine, threonine, valine or alanine residues, or a combination thereof). In yet another aspect, a knob-into-hole modification can be created by introducing only a knob modification, for example, by replacing an amino acid residue with a larger side chain than the original amino acid residue (e.g., a substitution of one or more tryptophan or tyrosine residues, or a combination thereof).

In some aspects, the knob-into-hole modification is in the binding interface of two Fc regions, the binding interface of two CH2 regions, the binding interface of two CH3 regions, the binding interface of a CL region and a CH1 region, or the binding interface of a VH region and a VL region. See, e.g., U.S. Pub. No. 2007/0178552, Int'l Pub. No. WO 96/027011, Int'l Pub. No. WO 98/050431 and Zhu et al., Protein Science 6:781-788, 1987.

In some aspects, the antigen binding polypeptide complex comprises one, two, three, four, five, six, seven, eight, nine, ten, or more knob-into-hole modifications.

Knob-into-hole modifications are well known and can be incorporated into the antigen binding polypeptide complexes of the invention using routine molecular biology and recombinant DNA techniques. See, e.g., U.S. Pub. No. 2003/0078385; Int'l Pub. No. WO 96/027011; Ridgway et al., Protein Eng., 9:617-621, 1996; and Merchant et al., Nat. Biotechnol., 16:677-681, 1998.

In some aspects, the knob-into-hole modification is an amino acid substitution. As used herein, such a substitution is described based on the EU numbering scheme of Kabat, which corresponds to the numbering in the Protein Data Bank (PDB).

In some aspects, the knob-into-hole modification is a knob substitution of S354C and/or T366W, based on the EU numbering scheme.

In some aspects, the knob-into-hole modification is a hole substitution of Y349C, T366S, L368A, Y407V, L234A, L235A, P329A, M428L, N433S, M252Y, S254T, T256E, or any combination thereof, based on the EU numbering scheme.

In some aspects, the knob-into-hole modifications are hole substitutions of Y349C, T366S, L368A and Y407V, based on the EU numbering scheme. In some aspects, the knob-into-hole modifications are a hole substitutions of L234A, L235A and P329A, based on the EU numbering scheme. In some aspects, the knob-into-hole modifications are hole substitutions of L234A and L235A, based on the EU numbering scheme. In some aspects, the knob-into-hole modifications are hole substitutions of M428L and N433S, based on the EU numbering scheme. In some aspects, the knob-into-hole modifications are hole substitutions of M252Y, S254T and T256E, based on the EU numbering scheme.

In some aspects, an antigen binding polypeptide complex is an IgG1 or IgG4 antibody and the knob-into-hole modifications are knob substitutions of S354C and T366W and hole substitutions of Y349C, T366S, L368A and Y407V.

In some aspects, the antigen binding polypeptide complex is an IgG1 or IgG4 antibody and the knob-into-hole modifications are hole substitutions of L234A, L235A and P329A.

In some aspects, the antigen binding polypeptide complex is an IgG1 or IgG4 antibody and the knob-into-hole modifications are hole substitutions of L234A and L235A.

In some aspects, the antigen binding polypeptide complex is an IgG1 or IgG4 antibody and the knob-into-hole modifications are hole substitutions of M428L and N433S.

In some aspects, the antigen binding polypeptide complex is an IgG1 or IgG4 antibody and the knob-into-hole modifications are hole substitutions of M252Y, S254T and T256E.

### Polypeptides, Polynucleotides, Vectors, Cells, and Protein Production Methods

In some aspects, the invention is directed to a polypeptide encoding a portion of an antigen binding polypeptide complex (e.g., an antibody or antigen binding fragment thereof) described herein.

In some aspects, the invention is directed to a polypeptide comprising an amino acid sequence of one or more of SEQ ID NOs:136, 138, 140, 142, 144, 146, 148, 150, 152, 154, 156, 158, 160, 162, 164, 166, 168, 170, 172, 174, 176, 178, 180, 182, 184, 186, 188, 190, 192, 194, 196, 198, 200, 202, 204, 206, 208, 210, 212, 214, 216, 218, 220, 222, 224, 226, 228, 230, 232, 234, 236, 238, 240, 242, 244, 246, 248, 250, 252, 254, 256, 258, 260, 262, 264, 266, 268, 270, 272, 274, 276, 278, 280, 282, 284, 286, 288, 290, 292, 294, 296, 298, 300, 302, 304, 306, 308, 310, 312, 314, 316, 318, 320, 322, 234, 326, 328, 330, 332, 334, 336, 338, 340, 342, 344, 346, 348, 350, 352, 354, 356, 358, 360, 362, 364, 366, 368, 370, 372, 374, 376, 378, 380, 382, 399, 412, 424, 426, 436, 438, 449, 459, 469, 479, 481, 483, 493, 511, 513, 515, 517, 529, 531, 533, 545, 557, 559, 561, 563, 565, 568, 570, 572, 574, 577, 579, 581, 585, 587, 589, 591, 593, 595, 597, 599, 601, 603, 608, 610, 612, 614, 616, 618, 628, 630, 632, 634, 652, 670, 688, 706, 724, 742, 760, 778, 796, 814 and 832; or an amino acid sequence having at least 90% identity or at least 95% identity to one or more of SEQ ID NOs:136, 138, 140, 142, 144, 146, 148, 150, 152, 154, 156, 158, 160, 162, 164, 166, 168, 170, 172, 174, 176, 178, 180, 182, 184, 186, 188, 190, 192, 194, 196, 198, 200, 202, 204, 206, 208, 210, 212, 214, 216, 218, 220, 222, 224, 226, 228, 230, 232, 234, 236, 238, 240, 242, 244, 246, 248, 250, 252, 254, 256, 258, 260, 262, 264, 266, 268, 270, 272, 274, 276, 278, 280, 282, 284, 286, 288, 290, 292, 294, 296, 298, 300, 302, 304, 306, 308, 310, 312, 314, 316, 318, 320, 322, 234, 326, 328, 330, 332, 334, 336, 338, 340, 342, 344, 346, 348, 350, 352, 354, 356, 358, 360, 362, 364, 366, 368, 370, 372, 374, 376, 378, 380, 382, 399, 412, 424, 426, 436, 438, 449, 459, 469, 479, 481, 483, 493, 511, 513, 515, 517, 529, 531, 533, 545, 557, 559, 561, 563, 565, 568, 570, 572, 574, 577, 579, 581, 585, 587, 589, 591, 593, 595, 597, 599, 601, 603, 608, 610, 612, 614, 616, 618, 628, 630, 632, 634, 652, 670, 688, 706, 724, 742, 760, 778, 796, 814 and 832. For example, the polypeptide may comprise an amino acid sequence having at least 90% (such as at least 91%, 92%, 93%, 94%, 95%, 96%, 97%, 98%, or 99%) identity to one or more of SEQ ID NOs: 136, 138, 140, 142, 144, 146, 148, 150, 152, 154, 156, 158, 160, 162, 164, 166, 168, 170, 172, 174, 176, 178, 180, 182, 184, 186, 188, 190, 192, 194, 196, 198, 200, 202, 204, 206, 208, 210, 212, 214, 216, 218, 220, 222, 224, 226, 228, 230, 232, 234, 236, 238, 240, 242, 244, 246, 248, 250, 252, 254, 256, 258, 260, 262, 264, 266, 268, 270, 272, 274, 276, 278, 280, 282, 284, 286, 288, 290, 292, 294, 296, 298, 300, 302, 304, 306, 308, 310, 312, 314, 316, 318, 320, 322, 234, 326, 328, 330, 332, 334, 336, 338, 340, 342, 344, 346, 348, 350, 352, 354, 356, 358, 360, 362, 364, 366, 368, 370, 372, 374, 376, 378, 380, 382, 399, 412, 424, 426, 436, 438, 449, 459, 469, 479, 481, 483, 493, 511, 513, 515, 517, 529, 531, 533, 545, 557, 559, 561, 563, 565, 568, 570, 572, 574, 577, 579, 581, 585, 587, 589, 591, 593, 595, 597, 599, 601, 603, 608, 610, 612, 614, 616, 618, 628, 630, 632, 634, 652, 670, 688, 706, 724, 742, 760, 778, 796, 814 and 832. For example, the polypeptide may comprise the amino acid sequence of one or more of SEQ ID NOs: 136, 138, 140, 142, 144, 146, 148, 150, 152, 154, 156, 158, 160, 162, 164, 166, 168, 170, 172, 174, 176, 178, 180, 182, 184, 186, 188, 190, 192, 194, 196, 198, 200, 202, 204, 206, 208, 210, 212, 214, 216, 218, 220, 222, 224, 226, 228, 230, 232, 234, 236, 238, 240, 242, 244, 246, 248, 250, 252, 254, 256, 258, 260, 262, 264, 266, 268, 270, 272, 274, 276, 278, 280, 282, 284, 286, 288, 290, 292, 294, 296, 298, 300, 302, 304, 306, 308, 310, 312, 314, 316, 318, 320, 322, 234, 326, 328, 330, 332, 334, 336, 338, 340, 342, 344, 346, 348, 350, 352, 354, 356, 358, 360, 362, 364, 366, 368, 370, 372, 374, 376, 378, 380, 382, 399, 412, 424, 426, 436, 438, 449, 459, 469, 479, 481, 483, 493, 511, 513, 515, 517, 529, 531, 533, 545, 557, 559, 561, 563, 565, 568, 570, 572, 574, 577, 579, 581, 585, 587, 589, 591, 593, 595, 597, 599, 601, 603, 608, 610, 612, 614, 616, 618, 628, 630, 632, 634, 652, 670, 688, 706, 724, 742, 760, 778, 796, 814 and 832. 10262| In some aspects, the invention is directed to a polypeptide comprising an amino acid sequence encoded by the polynucleotide sequence of one or more of SEQ ID NOs:137, 139, 141, 143, 145, 147, 149, 151, 153, 155, 157, 159, 161, 163, 165, 167, 169, 171, 173, 175, 177, 179, 181, 183, 185, 187, 189, 191, 193, 195, 197, 199, 201, 203, 205, 207, 209, 211, 213, 215, 217, 219, 221, 223, 225, 227, 229, 231, 233, 235, 237, 239, 241, 243, 245, 247, 249, 251, 253, 255, 257, 259, 261, 263, 265, 267, 269, 271, 273, 275, 277, 279, 281, 283, 285, 287, 289, 291, 293, 295, 297, 299, 301, 303, 305, 307, 309, 311, 313, 315, 317, 319, 321, 323, 235, 327, 329, 331, 333, 335, 337, 339, 341, 343, 345, 347, 349, 351, 353, 355, 357, 359, 361, 363, 365, 367, 369, 371, 373, 375, 377, 379, 381, 383, 401, 413, 425, 427, 437, 439, 450, 460, 470, 480, 482, 484, 494, 512, 514, 516, 518, 530, 532, 534, 546, 558, 560, 562, 564, 566, 569, 571, 573, 575, 578, 580, 582, 586, 588, 590, 592, 594, 596, 598, 600, 602, 604, 609, 611, 613, 615, 617, 619, 629, 631, 633, 635, 653, 671, 689, 707, 725. 743, 761, 779, 797, 815 and 833; or encoded by a polynucleotide having at least 90% identity or at least 95% identity to one or more of SEQ ID NOs:137, 139, 141, 143, 145, 147, 149, 151, 153, 155, 157, 159, 161, 163, 165, 167, 169, 171, 173, 175, 177, 179, 181, 183, 185, 187, 189, 191, 193, 195, 197, 199, 201, 203, 205, 207, 209, 211, 213, 215, 217, 219, 221, 223, 225, 227, 229, 231, 233, 235, 237, 239, 241, 243, 245, 247, 249, 251, 253, 255, 257, 259, 261, 263, 265, 267, 269, 271, 273, 275, 277, 279, 281, 283, 285, 287, 289, 291, 293, 295, 297, 299, 301, 303, 305, 307, 309, 311, 313, 315, 317, 319, 321, 323, 235, 327, 329, 331, 333, 335, 337, 339, 341, 343, 345, 347, 349, 351, 353, 355, 357, 359, 361, 363, 365, 367, 369, 371, 373, 375, 377, 379, 381, 383, 401, 413, 425, 427, 437, 439, 450, 460, 470, 480, 482, 484, 494, 512, 514, 516, 518, 530, 532, 534, 546, 558, 560, 562, 564, 566, 569, 571, 573, 575, 578, 580, 582, 586, 588, 590, 592, 594, 596, 598, 600, 602, 604, 609, 611, 613, 615, 617, 619, 629, 631, 633, 635, 653, 671, 689, 707, 725. 743, 761, 779, 797, 815 and 833. For example, the polypeptide may comprise an amino acid sequence encoded by a polynucleotide having at least 90% (such as at least 91%, 92%, 93%, 94%, 95%, 96%, 97%, 98%, or 99%) identity to one or more of SEQ ID NOs: 137, 139, 141, 143, 145, 147, 149, 151, 153, 155, 157, 159, 161, 163, 165, 167, 169, 171, 173, 175, 177, 179, 181, 183, 185, 187, 189, 191, 193, 195, 197, 199, 201, 203, 205, 207, 209, 211, 213, 215, 217, 219, 221, 223, 225, 227, 229, 231, 233, 235, 237, 239, 241, 243, 245, 247, 249, 251, 253, 255, 257, 259, 261, 263, 265, 267, 269, 271, 273, 275, 277, 279, 281, 283, 285, 287, 289, 291, 293, 295, 297, 299, 301, 303, 305, 307, 309, 311, 313, 315, 317, 319, 321, 323, 235, 327, 329, 331, 333, 335, 337, 339, 341, 343, 345, 347, 349, 351, 353, 355, 357, 359, 361, 363, 365, 367, 369, 371, 373, 375, 377, 379, 381, 383, 401, 413, 425, 427, 437, 439, 450, 460, 470, 480, 482, 484, 494, 512, 514, 516, 518, 530, 532, 534, 546, 558, 560, 562, 564, 566, 569, 571, 573, 575, 578, 580, 582, 586, 588, 590, 592, 594, 596, 598, 600, 602, 604, 609, 611, 613, 615, 617, 619, 629, 631, 633, 635, 653, 671, 689, 707, 725. 743, 761, 779, 797, 815 and 833. For example, the polypeptide may comprise an amino acid sequence encoded by a polynucleotide sequence of one or more of SEQ ID NOs: 137, 139, 141, 143, 145, 147, 149, 151, 153, 155, 157, 159, 161, 163, 165, 167, 169, 171, 173, 175, 177, 179, 181, 183, 185, 187, 189, 191, 193, 195, 197, 199, 201,203, 205, 207, 209, 211, 213, 215, 217, 219, 221, 223, 225, 227, 229, 231, 233, 235, 237, 239, 241, 243, 245, 247, 249, 251, 253, 255, 257, 259, 261, 263, 265, 267, 269, 271, 273, 275, 277, 279, 281, 283, 285, 287, 289, 291, 293, 295, 297, 299, 301, 303, 305, 307, 309, 311, 313, 315, 317, 319, 321, 323, 235, 327, 329, 331, 333, 335, 337, 339, 341, 343, 345, 347, 349, 351, 353, 355, 357, 359, 361, 363, 365, 367, 369, 371, 373, 375, 377, 379, 381, 383, 401, 413, 425, 427, 437, 439, 450, 460, 470, 480, 482, 484, 494, 512, 514, 516, 518, 530, 532, 534, 546, 558, 560, 562, 564, 566, 569, 571, 573, 575, 578, 580, 582, 586, 588, 590, 592, 594, 596, 598, 600, 602, 604, 609, 611, 613, 615, 617, 619, 629, 631, 633, 635, 653, 671, 689, 707, 725. 743, 761, 779, 797, 815 and 833.

In some aspects, the invention is directed to a polynucleotide encoding a portion of an antigen binding polypeptide complex (e.g., an antibody or antigen binding fragment thereof) described herein. As used herein, a “polynucleotide” includes DNA and RNA (e.g., mRNA).

In some aspects, the invention is directed to a polynucleotide comprising a polynucleotide sequence of one or more of SEQ ID NOs:137, 139, 141, 143, 145, 147, 149, 151, 153, 155, 157, 159, 161, 163, 165, 167, 169, 171, 173, 175, 177, 179, 181, 183, 185, 187, 189, 191, 193, 195, 197, 199, 201, 203, 205, 207, 209, 211, 213, 215, 217, 219, 221, 223, 225, 227, 229, 231, 233, 235, 237, 239, 241, 243, 245, 247, 249, 251, 253, 255, 257, 259, 261, 263, 265, 267, 269, 271, 273, 275, 277, 279, 281, 283, 285, 287, 289, 291, 293, 295, 297, 299, 301, 303, 305, 307, 309, 311, 313, 315, 317, 319, 321, 323, 235, 327, 329, 331, 333, 335, 337, 339, 341, 343, 345, 347, 349, 351, 353, 355, 357, 359, 361, 363, 365, 367, 369, 371, 373, 375, 377, 379, 381, 383, 401, 413, 425, 427, 437, 439, 450, 460, 470, 480, 482, 484, 494, 512, 514, 516, 518, 530, 532, 534, 546, 558, 560, 562, 564, 566, 569, 571, 573, 575, 578, 580, 582, 586, 588, 590, 592, 594, 596, 598, 600, 602, 604, 609, 611, 613, 615, 617, 619, 629, 631, 633, 635, 653, 671, 689, 707, 725. 743, 761, 779, 797, 815 and 833; or a polynucleotide having at least 90% identity or at least 95% identity to one or more of SEQ ID NOs:137, 139, 141, 143, 145, 147, 149, 151, 153, 155, 157, 159, 161, 163, 165, 167, 169, 171, 173, 175, 177, 179, 181, 183, 185, 187, 189, 191, 193, 195, 197, 199, 201, 203, 205, 207, 209, 211, 213, 215, 217, 219, 221, 223, 225, 227, 229, 231, 233, 235, 237, 239, 241, 243, 245, 247, 249, 251, 253, 255, 257, 259, 261, 263, 265, 267, 269, 271, 273, 275, 277, 279, 281, 283, 285, 287, 289, 291, 293, 295, 297, 299, 301, 303, 305, 307, 309, 311, 313, 315, 317, 319, 321, 323, 235, 327, 329, 331, 333, 335, 337, 339, 341, 343, 345, 347, 349, 351, 353, 355, 357, 359, 361, 363, 365, 367, 369, 371, 373, 375, 377, 379, 381, 383, 401, 413, 425, 427, 437, 439, 450, 460, 470, 480, 482, 484, 494, 512, 514, 516, 518, 530, 532, 534, 546, 558, 560, 562, 564, 566, 569, 571, 573, 575, 578, 580, 582, 586, 588, 590, 592, 594, 596, 598, 600, 602, 604, 609, 611, 613, 615, 617, 619, 629, 631, 633, 635, 653, 671, 689, 707, 725. 743, 761, 779, 797, 815 and 833. For example, the polynucleotide may comprise a polynucleotide sequence having at least 90% (such as at least 91%, 92%, 93%, 94%, 95%, 96%, 97%, 98%, or 99%) identity to one or more of SEQ ID NOs: 137, 139, 141, 143, 145, 147, 149, 151, 153, 155, 157, 159, 161, 163, 165, 167, 169, 171, 173, 175, 177, 179, 181, 183, 185, 187, 189, 191, 193, 195, 197, 199, 201, 203, 205, 207, 209, 211, 213, 215, 217, 219, 221, 223, 225, 227, 229, 231, 233, 235, 237, 239, 241, 243, 245, 247, 249, 251, 253, 255, 257, 259, 261, 263, 265, 267, 269, 271, 273, 275, 277, 279, 281, 283, 285, 287, 289, 291, 293, 295, 297, 299, 301, 303, 305, 307, 309, 311, 313, 315, 317, 319, 321, 323, 235, 327, 329, 331, 333, 335, 337, 339, 341, 343, 345, 347, 349, 351, 353, 355, 357, 359, 361, 363, 365, 367, 369, 371, 373, 375, 377, 379, 381, 383, 401, 413, 425, 427, 437, 439, 450, 460, 470, 480, 482, 484, 494, 512, 514, 516, 518, 530, 532, 534, 546, 558, 560, 562, 564, 566, 569, 571, 573, 575, 578, 580, 582, 586, 588, 590, 592, 594, 596, 598, 600, 602, 604, 609, 611, 613, 615, 617, 619, 629, 631, 633, 635, 653, 671, 689, 707, 725. 743, 761, 779, 797, 815 and 833. For example, the polynucleotide may comprise the polynucleotide sequence of one or more of SEQ ID NOs: 137, 139, 141, 143, 145, 147, 149, 151, 153, 155, 157, 159, 161, 163, 165, 167, 169, 171, 173, 175, 177, 179, 181, 183, 185, 187, 189, 191, 193, 195, 197, 199, 201, 203, 205, 207, 209, 211, 213, 215, 217, 219, 221, 223, 225, 227, 229, 231, 233, 235, 237, 239, 241, 243, 245, 247, 249, 251, 253, 255, 257, 259, 261, 263, 265, 267, 269, 271, 273, 275, 277, 279, 281, 283, 285, 287, 289, 291, 293, 295, 297, 299, 301, 303, 305, 307, 309, 311, 313, 315, 317, 319, 321, 323, 235, 327, 329, 331, 333, 335, 337, 339, 341, 343, 345, 347, 349, 351, 353, 355, 357, 359, 361, 363, 365, 367, 369, 371, 373, 375, 377, 379, 381, 383, 401, 413, 425, 427, 437, 439, 450, 460, 470, 480, 482, 484, 494, 512, 514, 516, 518, 530, 532, 534, 546, 558, 560, 562, 564, 566, 569, 571, 573, 575, 578, 580, 582, 586, 588, 590, 592, 594, 596, 598, 600, 602, 604, 609, 611, 613, 615, 617, 619, 629, 631, 633, 635, 653, 671, 689, 707, 725. 743, 761, 779, 797, 815 and 833.

In some aspects, the invention is directed to a polynucleotide encoding a polypeptide of one or more of SEQ ID NOs:136, 138, 140, 142, 144, 146, 148, 150, 152, 154, 156, 158, 160, 162, 164, 166, 168, 170, 172, 174, 176, 178, 180, 182, 184, 186, 188, 190, 192, 194, 196, 198, 200, 202, 204, 206, 208, 210, 212, 214, 216, 218, 220, 222, 224, 226, 228, 230, 232, 234, 236, 238, 240, 242, 244, 246, 248, 250, 252, 254, 256, 258, 260, 262, 264, 266, 268, 270, 272, 274, 276, 278, 280, 282, 284, 286, 288, 290, 292, 294, 296, 298, 300, 302, 304, 306, 308, 310, 312, 314, 316, 318, 320, 322, 234, 326, 328, 330, 332, 334, 336, 338, 340, 342, 344, 346, 348, 350, 352, 354, 356, 358, 360, 362, 364, 366, 368, 370, 372, 374, 376, 378, 380, 382, 399, 412, 424, 426, 436, 438, 449, 459, 469, 479, 481, 483, 493, 511, 513, 515, 517, 529, 531, 533, 545, 557, 559, 561, 563, 565, 568, 570, 572, 574, 577, 579, 581, 585, 587, 589, 591, 593, 595, 597, 599, 601, 603, 608, 610, 612, 614, 616, 618, 628, 630, 632, 634, 652, 670, 688, 706, 724, 742, 760, 778, 796, 814 and 832; or a polypeptide having at least 90% identity or at least 95% identity to one or more of SEQ ID NOs:136, 138, 140, 142, 144, 146, 148, 150, 152, 154, 156, 158, 160, 162, 164, 166, 168, 170, 172, 174, 176, 178, 180, 182, 184, 186, 188, 190, 192, 194, 196, 198, 200, 202, 204, 206, 208, 210, 212, 214, 216, 218, 220, 222, 224, 226, 228, 230, 232, 234, 236, 238, 240, 242, 244, 246, 248, 250, 252, 254, 256, 258, 260, 262, 264, 266, 268, 270, 272, 274, 276, 278, 280, 282, 284, 286, 288, 290, 292, 294, 296, 298, 300, 302, 304, 306, 308, 310, 312, 314, 316, 318, 320, 322, 234, 326, 328, 330, 332, 334, 336, 338, 340, 342, 344, 346, 348, 350, 352, 354, 356, 358, 360, 362, 364, 366, 368, 370, 372, 374, 376, 378, 380, 382, 399, 412, 424, 426, 436, 438, 449, 459, 469, 479, 481, 483, 493, 511, 513, 515, 517, 529, 531, 533, 545, 557, 559, 561, 563, 565, 568, 570, 572, 574, 577, 579, 581, 585, 587, 589, 591, 593, 595, 597, 599, 601, 603, 608, 610, 612, 614, 616, 618, 628, 630, 632, 634, 652, 670, 688, 706, 724, 742, 760, 778, 796, 814 and 832. For example, the polynucleotide may encode a polypeptide comprising an amino acid sequence having at least 90% (such as at least 91%, 92%, 93%, 94%, 95%, 96%, 97%, 98%, or 99%) identity to one or more of SEQ ID NOs: 136, 138, 140, 142, 144, 146, 148, 150, 152, 154, 156, 158, 160, 162, 164, 166, 168, 170, 172, 174, 176, 178, 180, 182, 184, 186, 188, 190, 192, 194, 196, 198, 200, 202, 204, 206, 208, 210, 212, 214, 216, 218, 220, 222, 224, 226, 228, 230, 232, 234, 236, 238, 240, 242, 244, 246, 248, 250, 252, 254, 256, 258, 260, 262, 264, 266, 268, 270, 272, 274, 276, 278, 280, 282, 284, 286, 288, 290, 292, 294, 296, 298, 300, 302, 304, 306, 308, 310, 312, 314, 316, 318, 320, 322, 234, 326, 328, 330, 332, 334, 336, 338, 340, 342, 344, 346, 348, 350, 352, 354, 356, 358, 360, 362, 364, 366, 368, 370, 372, 374, 376, 378, 380, 382, 399, 412, 424, 426, 436, 438, 449, 459, 469, 479, 481, 483, 493, 511, 513, 515, 517, 529, 531, 533, 545, 557, 559, 561, 563, 565, 568, 570, 572, 574, 577, 579, 581, 585, 587, 589, 591, 593, 595, 597, 599, 601, 603, 608, 610, 612, 614, 616, 618, 628, 630, 632, 634, 652, 670, 688, 706, 724, 742, 760, 778, 796, 814 and 832. For example, the polynucleotide may encode a polypeptide comprising the amino acid sequence of one or more of SEQ ID NOs: 136, 138, 140, 142, 144, 146, 148, 150, 152, 154, 156, 158, 160, 162, 164, 166, 168, 170, 172, 174, 176, 178, 180, 182, 184, 186, 188, 190, 192, 194, 196, 198, 200, 202, 204, 206, 208, 210, 212, 214, 216, 218, 220, 222, 224, 226, 228, 230, 232, 234, 236, 238, 240, 242, 244, 246, 248, 250, 252, 254, 256, 258, 260, 262, 264, 266, 268, 270, 272, 274, 276, 278, 280, 282, 284, 286, 288, 290, 292, 294, 296, 298, 300, 302, 304, 306, 308, 310, 312, 314, 316, 318, 320, 322, 234, 326, 328, 330, 332, 334, 336, 338, 340, 342, 344, 346, 348, 350, 352, 354, 356, 358, 360, 362, 364, 366, 368, 370, 372, 374, 376, 378, 380, 382, 399, 412, 424, 426, 436, 438, 449, 459, 469, 479, 481, 483, 493, 511, 513, 515, 517, 529, 531, 533, 545, 557, 559, 561, 563, 565, 568, 570, 572, 574, 577, 579, 581, 585, 587, 589, 591, 593, 595, 597, 599, 601, 603, 608, 610, 612, 614, 616, 618, 628, 630, 632, 634, 652, 670, 688, 706, 724, 742, 760, 778, 796, 814 and 832.

In other aspects, the invention is directed to a vector comprising one or more polynucleotides described herein.

In yet other aspects, the invention is directed to a host cell comprising one or more polynucleotides and/or vectors described herein. For example, the host cell may comprise one or more polynucleotide described herein. For example, the host cell may comprise one or more vectors described herein.

As used herein, the term “host cell” can be any type of cell, e.g., a primary cell, a cell in culture, or a cell from a cell line. In some aspects, the term “host cell” refers to a cell containing a foreign gene [e.g., a cell subjected to gene delivery or transfected with a polynucleotide (e.g., DNA or mRNA) encoding the gene] and the progeny or potential progeny of such a cell. Progeny of such a cell may not be identical to the parent cell transfected with the nucleic acid molecule, e.g., due to mutations or environmental influences that may occur in succeeding generations or integration of the nucleic acid molecule into the host cell genome.

Methods which are well known to those skilled in the art can be used to construct vectors encoding antigen binding polypeptide complexes (e.g., CDR, VH, VL, heavy chain and/or light chain coding sequences and appropriate transcriptional and translational control signals). These methods include, for example, in vitro recombinant DNA techniques, synthetic techniques, and in vivo genetic recombination.

A vector can be transferred to a host cell by conventional techniques and the resulting cells can then be cultured by conventional techniques to produce an antigen binding polypeptide complex comprising, e.g., six CDRs, VH, VL, VH and VL, heavy chain, light chain, or heavy and light chain, or a domain thereof (e.g., one or more CDRs, VH, VL, VH and VL, heavy chain, or light chain). Thus, provided herein are host cells containing a polynucleotide encoding an antigen binding polypeptide complex comprising, e.g., comprising six CDRs, VH, VL, VH and VL, heavy chain, light chain, or heavy and light chain, or a domain thereof (e.g., one or more CDRs, VH, VL, VH and VL, heavy chain, or light chain), operably linked to a promoter for expression of such sequences in the host cell. In some aspects, vectors encoding both heavy and light chains, or a domain thereof, individually, can be co-expressed in the host cell for expression. In some aspects, a host cell contains a vector comprising a polynucleotide encoding both a heavy chain and light chain, or a domain thereof. In some aspects, a host cell contains two different vectors, a first vector comprising a polynucleotide encoding a heavy chain or a domain thereof, and a second vector comprising a polynucleotide encoding a light chain or a domain thereof. In some aspects, a first host cell comprises a first vector comprising a polynucleotide encoding a heavy chain or a domain thereof, and a second host cell comprises a second vector comprising a polynucleotide encoding a light chain or a domain thereof. In some aspects, provided herein is a population of host cells comprising such a first host cell and such a second host cell.

In some aspects, provided herein is a population of vectors comprising a first vector comprising a polynucleotide encoding a light chain or domain thereof, and a second vector comprising a polynucleotide encoding a heavy chain or domain thereof. Alternatively, a single vector can be used which encodes, and is capable of expressing, both heavy and light chain polypeptides or a domain thereof.

A variety of host-vector systems can be utilized to express the polypeptides and polypeptide complexes described herein. Such host-vector systems represent vehicles by which the coding sequences of interest can be produced and subsequently purified, but also represent cells which can, when transformed or transfected with the appropriate nucleotide coding sequences, express a polypeptide or polypeptide complex described herein in situ. These include but are not limited to microorganisms such as bacteria (e.g., E. coli and B. subtilis) transformed with recombinant bacteriophage DNA, plasmid DNA or cosmid DNA expression vectors containing antibody coding sequences; yeast (e.g., Saccharomyces pichia) transformed with recombinant yeast expression vectors containing antibody coding sequences; insect cell systems infected with recombinant virus expression vectors (e.g., baculovirus) containing antibody coding sequences; plant cell systems (e.g., green algae such as Chlamydomonas reinhardtii) infected with recombinant virus expression vectors (e.g., cauliflower mosaic virus, CaMV; tobacco mosaic virus, TMV) or transformed with recombinant plasmid expression vectors (e.g., Ti plasmid) containing antibody coding sequences; or mammalian cell systems (e.g., COS (e.g., COS1 or COS), CHO, BHK, MDCK, HEK 293, NS0, PER.C6, VERO, CRL7O3O, HsS78Bst, HeLa, and NIH 3T3, HEK-293T, HepG2, SP210, R1.1, B-W, L-M, BSC1, BSC40, YB/20, and BMT10 cells) harboring recombinant expression constructs containing promoters derived from the genome of mammalian cells (e.g., metallothionein promoter) or from mammalian viruses (e.g., the adenovirus late promoter; the vaccinia virus 7.5K promoter). In some aspects, cells for expressing polypeptide or polypeptide complexes described herein are CHO cells, for example CHO cells from the CHO GS System™ (Lonza). In some aspects, cells for expressing polypeptides or polypeptide complexes of the invention are human cells, e.g., human cell lines. In some aspects, a mammalian expression vector is pOptiVEC™ or pcDNA3.3. In some aspects, bacterial cells such as Escherichia coli, or eukaryotic cells (e.g., mammalian cells) are used for the expression of recombinant polypeptides. For example, mammalian cells such as Chinese hamster ovary (CHO) cells in conjunction with a vector such as the major intermediate early gene promoter element from human cytomegalovirus is an effective expression system for polypeptides (Foecking M K & Hofstetter H (1986) Gene 45: 101-105; and Cockett M I et al., (1990) Biotechnology 8: 662-667). In some aspects, polypeptides or polypeptide complexes described herein are produced by HEK-293T cells.

In addition, a host cell strain can be chosen which modulates the expression of the inserted sequences, or modifies and processes the gene product in the specific fashion desired. Such modifications (e.g., glycosylation) and processing (e.g., cleavage) of protein products can contribute to the function of the protein. To this end, eukaryotic host cells which possess the cellular machinery for proper processing of the primary transcript, glycosylation, and phosphorylation of the gene product can be used. Such mammalian host cells include but are not limited to CHO, VERO, BHK, Hela, MDCK, HEK 293, NIH 3T3, W138, BT483, Hs578T, HTB2, BT20 and T47D, NS0 (a murine myeloma cell line that does not endogenously produce any immunoglobulin chains), CRL7O3O, COS (e.g., COS1 or COS), PER.C6, VERO, HsS78Bst, HEK-293T, HepG2, SP210, R1.1, B-W, L-M, BSC1, BSC40, YB/20, BMT10 and HsS78Bst cells.

Once a polypeptide or polypeptide complex described herein has been produced by recombinant expression, it can be purified by any method known in the art for purification of a protein or immunoglobulin molecule, for example, by chromatography (e.g., ion exchange, affinity, particularly by affinity for the specific antigen after Protein A, and size exclusion chromatography), centrifugation, differential solubility, or by any other standard technique for the purification of proteins. Further, the polypeptides or polypeptide complexes described herein can be fused to heterologous polypeptide sequences described herein (e.g., tags) or otherwise known in the art to facilitate purification.

In some aspects, a polypeptide or polypeptide complex described herein is isolated or purified. Generally, an isolated polypeptide or polypeptide complex is one that is substantially free of other polypeptides or polypeptide complexes with different antigenic specificities. For example, in some aspects, a preparation of a polypeptide or polypeptide complex described herein is substantially free of cellular material and/or chemical precursors.

### Pharmaceutical Compositions and Kits

In some aspects, the invention is directed to a pharmaceutical composition comprising an antigen binding polypeptide complex (e.g., antibody or antigen binding fragment thereof), polypeptide(s), polynucleotide(s), vector(s), or cell(s) described herein.

In some aspects, the invention is directed to a pharmaceutical composition comprising (1) an antigen binding polypeptide complex (e.g., antibody or antigen binding fragment thereof), polynucleotide, vector, or cell described herein, and (2) a pharmaceutically acceptable carrier. The term “pharmaceutically acceptable carrier” includes any and all solvents, co-solvents, complexing agents, dispersion media, coatings, antibacterial and antifungal agents, isotonic and absorption delaying agents, and the like, which are not biologically or otherwise undesirable. The use of such media and agents for pharmaceutically active substances is known in the art. Except insofar as any conventional media or agent is incompatible with the active ingredient, its use in the therapeutic formulations is contemplated. Supplementary active ingredients can also be incorporated into the pharmaceutical compositions of the invention. In addition, various excipients, such as are commonly used in the art, can be included. These and other such compounds are described in the literature, e.g., in the Merck Index, Merck & Company, Rahway, NJ. Considerations for the inclusion of various components in pharmaceutical compositions are described, e.g., in Gilman et al. (Eds.) (2010); Goodman and Gilman's: The Pharmacological Basis of Therapeutics, 12th Ed., The McGraw-Hill Companies. In some aspects, the pharmaceutical composition is for parenteral, intravenous or subcutaneous administration.

In other aspects, the invention is directed to a kit comprising an antigen binding polypeptide complex (e.g., antibody or antigen binding fragment thereof), polypeptide(s), polynucleotide(s), vector(s), cell(s), or pharmaceutical composition described herein, or a combination thereof. Once a pharmaceutical composition has been formulated, it can be stored in sterile vials as a solution, suspension, gel, emulsion, solid, crystal, or as a dehydrated or lyophilized powder. Such formulations may be stored either in a ready-to-use form or in a form (e.g., lyophilized) that is reconstituted prior to administration. In some aspects, the invention provides kits for producing a single-dose administration unit. In some aspects, the kits of the invention can contain both a first container having a dried protein and a second container having an aqueous formulation. In some aspects, kits containing single and multi-chambered pre-filled syringes (e.g., liquid syringes and lyosyringes) are also provided. In some aspects, the kit contains components for intravenous or subcutaneous administration.

### Methods of Use

In some aspects, the invention is directed to certain methods of use of an antigen binding polypeptide complex (e.g., an antibody or antigen binding fragment thereof), polypeptide(s), polynucleotide(s), vector(s), cell(s), or pharmaceutical composition described herein, or a combination thereof. Any of the antigen binding polypeptide complex structures described herein targeting one or more of the targets described herein may be used in any of the methods and uses of the invention.

In some aspects, the invention is directed to a method of treating or preventing a disease or condition, comprising administering to a subject in need thereof an antigen binding polypeptide complex (e.g., antibody or antigen binding fragment thereof), polypeptide(s), polynucleotide(s), vector(s), cell(s), or pharmaceutical composition described herein, or a combination thereof. In some aspects, the invention is directed to a method of treating or preventing a disease or condition, comprising administering to a subject in need thereof a therapeutically effective amount of an antigen binding polypeptide complex (e.g., antibody or antigen binding fragment thereof), polypeptide(s), polynucleotide(s), vector(s), cell(s), or pharmaceutical composition described herein, or a combination thereof. The present invention further provides an antigen binding polypeptide complex (e.g., antibody or antigen binding fragment thereof), polypeptide(s), polynucleotide(s), vector(s), cell(s), or pharmaceutical composition described herein, or a combination thereof, for use in treating or preventing a disease or condition in a subject. The present invention further provides the use of an antigen binding polypeptide complex (e.g., antibody or antigen binding fragment thereof), polypeptide(s), polynucleotide(s), vector(s), cell(s), or pharmaceutical composition described herein, or a combination thereof in the manufacture of a medicament for the treatment or prevention of a disease or condition in a subject.

In some aspects, the invention is directed to a method of treating cancer comprising administering to a subject in need thereof an antigen binding polypeptide complex (e.g., antibody or antigen binding fragment thereof), polypeptide(s), polynucleotide(s), vector(s), cell(s) or pharmaceutical composition described herein, or a combination thereof. In some aspects, the cancer is a solid cancer. In some aspects, the cancer is breast cancer, lung cancer, gastric cancer, prostate cancer, cervical cancer, urothelial cancer, or pancreatic cancer. In some aspects, the cancer is a hematological cancer. In some aspects, the hematological cancer is leukemia or lymphoma. In some aspects, the leukemia or lymphoma is B cell leukemia, B cell lymphoma, diffuse large B-cell lymphoma (DLBCL) Follicular lymphoma, Chronic lymphocytic leukemia (CLL)/small lymphocytic lymphoma (SLL), Mantle cell lymphoma (MCL), Burkitt lymphoma, Lymphoplasmacytic lymphoma (Waldenstrom macroglobulinemia), prolymphocytic leukemia (PLL), or hairy cell leukemia (HCL).

The present invention further provides an antigen binding polypeptide complex (e.g., antibody or antigen binding fragment thereof), polypeptide(s), polynucleotide(s), vector(s), cell(s), or pharmaceutical composition described herein, or a combination thereof, for use in treating or preventing a cancer in a subject. The present invention further provides the use of an antigen binding polypeptide complex (e.g., antibody or antigen binding fragment thereof), polypeptide(s), polynucleotide(s), vector(s), cell(s), or pharmaceutical composition described herein, or a combination thereof in the manufacture of a medicament for the treatment or prevention of a cancer in a subject.

In some aspects, the invention is directed to a method of treating or preventing a viral infection comprising administering to a subject in need thereof an antigen binding polypeptide complex (e.g., antibody or antigen binding fragment thereof), polypeptide(s), polynucleotide(s), vector(s), cell(s) or pharmaceutical composition described herein, or a combination thereof. In some aspects, the invention is directed to an antigen binding polypeptide complex (e.g., antibody or antigen binding fragment thereof), polypeptide(s), polynucleotide(s), vector(s), cell(s), or pharmaceutical composition described herein, or a combination thereof, for use in treating or preventing a viral infection in a subject. The present invention further provides the use of an antigen binding polypeptide complex (e.g., antibody or antigen binding fragment thereof), polypeptide(s), polynucleotide(s), vector(s), cell(s), or pharmaceutical composition described herein, or a combination thereof in the manufacture of a medicament for the treatment or prevention of a viral infection in a subject. 10284| As used herein, the term “subject” means a human or a non-human mammal, e.g., a dog, a cat, a mouse, a rat, a cow, a sheep, a pig, a goat, a non-human primate or a bird, e.g., a chicken, as well as any other vertebrate or invertebrate. In some aspects, the subject is a human. In some aspects, the subject is a veterinary animal. In some aspects, the subject is a mammal.

As used herein, the terms “treat” or “treatment” refer to therapeutic or palliative measures. Beneficial or desired clinical results include, but are not limited to, alleviation, in whole or in part, of symptoms associated with a disease or disorder or condition, diminishment of the extent of disease, stabilized (i.e., not worsening) state of disease, delay or slowing of disease progression, amelioration or palliation of the disease state (e.g., one or more symptoms of the disease), and remission (whether partial or total), whether detectable or undetectable. “Treatment” can also mean prolonging survival as compared to expected survival if not receiving treatment.

As used herein, a “therapeutically effective amount” is an amount of an antigen binding polypeptide complex (e.g., an antibody or antigen binding fragment thereof) that is sufficient to achieve the desired effect and can vary according to the nature and severity of the disease condition, and the potency of the polypeptide or polypeptide complex. In some aspects, an antigen binding polypeptide complex of the invention can be delivered by administering a polynucleotide, vector, cell that encodes the antigen binding polypeptide complex. In some aspects, an antigen binding polypeptide complex thereof can be delivered by administering a pharmaceutical composition containing the polypeptide or polypeptide complex. A therapeutic effect is the relief, to some extent, of one or more of the symptoms of the disease, and can include curing a disease. “Curing” means that the symptoms of active disease are eliminated. However, certain long-term or permanent effects of the disease can exist even after a cure is obtained.

Clauses relating to aspects of the invention:
1. An antigen binding polypeptide complex comprising a first polypeptide, a second polypeptide, and a third polypeptide;
(a) wherein the first polypeptide has a structure represented by:
   VL1-L1-VH1-L2-Fc;
   VH1-L3-VL1-L4-Fc;
   VH1-L1-VL1-L2-Fc; or
   VL1-L3-VH1-L4-Fc;
wherein the second polypeptide has a structure represented by:
   VH2-L5-VH3-L6-CH1-L7-Fc; or
   VH3-L5-VH2-L6-CH1-L7-Fc;
wherein the third polypeptide has a structure represented by:
   VL2-L8-VL3-L9-CL; or
   VL3-L8-VL2-L9-CL;
or
wherein the first polypeptide has a structure represented by:
   VL1-L1-VL2-L2-VH2-L3-VH1-L4-Fc;
   VL1-L1-VH2-L2-VL2-L3-VH1-L4-Fc;
   VH1-L5-VH2-L6-VL2-L7-VL1-L8-Fc; or
   VH1-L5-VL2-L6-VH2-L7-VL1-L8-Fc;
wherein the second polypeptide has a structure represented by:
   VH3-L9-VH4-L10-CH1-L11-Fc or
   VH4-L9-VH3-L10-CH1-L11-Fc;
wherein the third polypeptide has a structure represented by:
   VL3-L12-VL4-L13-CL; or
   VL4-L12-VL3-L13-CL;
wherein
VL1 is a first immunoglobulin light chain variable region; VL2 is a second immunoglobulin light chain variable region; VL3 is a third immunoglobulin light chain variable region; VL4 is a fourth immunoglobulin light chain variable region; VH1 is a first immunoglobulin heavy chain variable region; VH2 is a second immunoglobulin heavy chain variable region; VH3 is a third immunoglobulin heavy chain variable region; VH4 is a fourth immunoglobulin heavy chain variable region; Fc is a region comprising an immunoglobulin heavy chain constant region 2 (CH2), an immunoglobulin heavy chain constant region 3 (CH3), and optionally, an immunoglobulin hinge; CH1 is an immunoglobulin heavy chain constant region 1; CL is an immunoglobulin light chain constant region; and L1-L13 are amino acid linkers; or
(b) wherein the first polypeptide has a structure represented by:
   VL1-L1-VH1-L2-Fc;
   VH1-L3-VL1-L4-Fc;
   VH1-L1-VL1-L2-Fc; or
   VL1-L3-VH1-L4-Fc;
wherein the second polypeptide has a structure represented by:
   VH2-L5-VH3-L6-CH1-L7-Fc; or
   VH3-L5-VH2-L6-CH1-L7-Fc;
wherein the third polypeptide has a structure represented by:
   VL2-L8-VL3-L9-CL; or
   VL3-L8-VL2-L9-CL;
or
wherein the first polypeptide has a structure represented by:
   VL1-L1-VL2-L2-VH2-L3-VH1-L4-Fc;
   VL1-L1-VH2-L2-VL2-L3-VH1-L4-Fc;
   VH1-L5-VH2-L6-VL2-L7-VL1-L8-Fc; or
   VH1-L5-VL2-L6-VH2-L7-VL1-L8-Fc;
wherein the second polypeptide has a structure represented by:
   VH3-L9-VH4-CH1-Fc; or
   VH4-L9-VH3-CH1-Fc;
wherein the third polypeptide has a structure represented by:
   VL3-L10-VL4-CL; or
   VL4-L10-VL3-CL;
wherein
   VL1 is a first immunoglobulin light chain variable region; VL2 is a second immunoglobulin light chain variable region; VL3 is a third immunoglobulin light chain variable region; VL4 is a fourth immunoglobulin light chain variable region; VH1 is a first immunoglobulin heavy chain variable region; VH2 is a second immunoglobulin heavy chain variable region; VH3 is a third immunoglobulin heavy chain variable region; VH4 is a fourth immunoglobulin heavy chain variable region; Fc is a region comprising an immunoglobulin heavy chain constant region 2 (CH2), an immunoglobulin heavy chain constant region 3 (CH3), and optionally, an immunoglobulin hinge; CH1 is an immunoglobulin heavy chain constant region 1; CL is an immunoglobulin light chain constant region; and L1-L10 are amino acid linkers.
2. The antigen binding polypeptide complex according to clause 1, wherein the first polypeptide has a structure represented by: VL1-L1-VH1-L2-Fc; or VH1-L3-VL1-L4-Fc; wherein the second polypeptide has a structure represented by: VH2-L5-VH3-L6-CH1-L7-Fc; and wherein the third polypeptide has a structure represented by: VL2-L8-VL3-L9-CL.
3. The antigen binding polypeptide complex according to clause 1, wherein
(a) the first polypeptide has a structure represented by VL1-L1-VL2-L2-VH2-L3-VH1-L4-Fc or VH1-L5-VL2-L6-VH2-L7-VL1-L8-Fc; wherein the second polypeptide has a structure represented by VH3-L9-VH4-L10-CH1-L11-Fc; and wherein the third polypeptide has a structure represented by VL3-L12-VL4-L13-CL, or
(b) the first polypeptide has a structure represented by VL1-L1-VL2-L2-VH2-L3-VH1-L4-Fc or VH1-L5-VL2-L6-VH2-L7-VL1-L8-Fc; wherein the second polypeptide has a structure represented by VH3-L9-VH4-CH1-Fc; and wherein the third polypeptide has a structure represented by VL3-L10-VL4-CL.
4. The antigen binding polypeptide complex according to clause 1, wherein
(a) the first polypeptide has a structure represented by: VL1-L1-VL2-L2-VH2-L3-VH1-L4-Fc; or VH1-L5-VH2-L6-VL2-L7-VL1-L8-Fc; wherein the second polypeptide has a structure represented by: VH3-L9-VH4-L10-CH1-L11-Fc; wherein the third polypeptide has a structure represented by: VL3-L12-VL4-L13-CL or
(b) the first polypeptide has a structure represented by: VL1-L1-VL2-L2-VH2-L3-VH1-L4-Fc; or VH1-L5-VH2-L6-VL2-L7-VL1-L8-Fc; wherein the second polypeptide has a structure represented by: VH3-L9-VH4-CH1-Fc; wherein the third polypeptide has a structure represented by: VL3-L10-VL4-CL.
5. An antigen binding polypeptide complex comprising a first polypeptide, a second polypeptide, a third polypeptide, and a fourth polypeptide;
wherein the first polypeptide has a structure represented by:
   VL1-L1-CL or
   VH1-L1-CL;
wherein the second polypeptide has a structure represented by:
   VH1-L2-CH1-L3-Fc or
   VL1-L2-CH1-L3-Fc;
wherein the third polypeptide has a structure represented by:
   VH2-L4-VH3-L5-CH1-L6-Fc or
   VH3-L4-VH2-L5-CH1-L6-Fc;
wherein the fourth polypeptide has a structure represented by:
   VL2-L7-VL3-L8-CL or
   VL3-L7-VL2-L8-CL;
wherein:
VL1 is a first immunoglobulin light chain variable region; VL2 is a second immunoglobulin light chain variable region; VL3 is a third immunoglobulin light chain variable region; VH1 is a first immunoglobulin heavy chain variable region; VH2 is a second immunoglobulin heavy chain variable region; VH3 is a third immunoglobulin heavy chain variable region; Fc is a region comprising an immunoglobulin heavy chain constant region 2 (CH2), an immunoglobulin heavy chain constant region 3 (CH3), and optionally, an immunoglobulin hinge; CH1 is an immunoglobulin heavy chain constant region 1; CL is an immunoglobulin light chain constant region; and L1-L8 are amino acid linkers.
6. The antigen binding polypeptide complex according to clause 5, wherein the first polypeptide has a structure represented by: VL1-L1-CL; wherein the second polypeptide has a structure represented by: VH1-L2-CH1-L3-Fc; wherein the third polypeptide has a structure represented by: VH2-L4-VH3-L5-CH1-L6-Fc; wherein the fourth polypeptide has a structure represented by: VL2-L7-VL3-L8-CL.
7. An antigen binding polypeptide complex comprising a first polypeptide and a second polypeptide;
wherein the first polypeptide has a structure represented by:
   VL1-L1-VL2-L2-VH2-L3-VH1-L4-Fc;
   VL1-L1-VH2-L2-VL2-L3-VH1-L4-Fc;
   VH1-L5-VH2-L6-VL2-L7-VL1-L8-Fc;
   VH1-L5-VL2-L6-VH2-L7-VL1-L8-Fc;
   VL1-L1-VL2-L2-VH2-L3-VH1-L4-CH1-L5-Fc;
   VL1-L1-VH2-L2-VL2-L3-VH1-L4-CH1-L5-Fc;
   VH1-L6-VH2-L7-VL2-L8-VL1-L9-CH1-L10-Fc;
   VH1-L6-VL2-L7-VH2-L8-VL1-L9-CH1-L10-Fc;
   VL1-L11-VL2-L12-VH2-L13-VH1-L14-CL-L15-Fc;
   VL1-L11-VH2-L12-VL2-L13-VH1-L14-CL-L15-Fc;
   VH1-L16-VH2-L17-VL2-L18-VL1-L19-CL-L20-Fc;
   VH1-L16-VL2-L17-VH2-L18-VL1-L19-CL-L20-Fc;
   VL1-L21-VL2-L22-VH2-L23-VH1-L24-CH1-L25-CL-L26-Fc;
   VL1-L21-VH2-L22-VL2-L23-VH1-L24-CH1-L25-CL-L26-Fc;
   VH1-L27-VH2-L28-VL2-L29-VL1-L30-CH1-L31-CL-L32-Fc;
   VH1-L27-VL2-L28-VH2-L29-VL1-L30-CH1-L31-CL-L32-Fc;
   VL1-L33-VL2-L34-VH2-L35-VH1-L36-CL-L37-CH1-L38-Fc;
   VL1-L33-VH2-L34-VL2-L35-VH1-L36-CL-L37-CH1-L38-Fc;
   VH1-L39-VH2-L40-VL2-L41-VL1-L42-CL-L43-CH1-L44-Fc; or
   VH1-L39-VL2-L40-VH2-L41-VL1-L42-CL-L43-CH1-L44-Fc;
wherein the second polypeptide has a structure represented by:
   VL3-L9-VL4-L10-VH4-L11-VH3-L12-Fc;
   VL3-L9-VH4-L10-VL4-L11-VH3-L12-Fc;
   VH3-L13-VH4-L14-VL4-L15-VL3-L16-Fc;
   VH3-L13-VL4-L14-VH4-L15-VL3-L16-Fc
   VL3-L45-VL4-L46-VH4-L47-VH3-L48-CH1-L49-Fc;
   VL3-L45-VH4-L46-VL4-L47-VH3-L48-CH1-L49-Fc;
   VH3-L50-VH4-L51-VL4-L52-VL3-L53-CH1-L54-Fc;
   VH3-L50-VL4-L51-VH4-L52-VL3-L53-CH1-L54-Fc;
   VL3-L55-VL4-L56-VH4-L57-VH3-L58-CL-L59-Fc;
   VL3-L55-VH4-L56-VL4-L57-VH3-L58-CL-L59-Fc;
   VH3-L60-VH4-L61-VL4-L62-VL3-L63-CL-L64-Fc;
   VH3-L60-VL4-L61-VH4-L62-VL3-L63-CL-L64-Fc;
   VL3-L65-VL4-L66-VH4-L67-VH3-L68-CH1-L69-CL-L70-Fc;
   VL3-L65-VH4-L66-VL4-L67-VH3-L68-CH1-L69-CL-L70-Fc;
   VH3-L71-VH4-L72-VL4-L73-VL3-L74-CH1-L75-CL-L76-Fc;
   VH3-L71-VL4-L72-VH4-L73-VL3-L74-CH1-L75-CL-L76-Fc;
   VL3-L77-VL4-L78-VH4-L79-VH3-L80-CL-L81-CH1-L82-Fc;
   VL3-L77-VH4-L78-VL4-L79-VH3-L80-CL-L81-CH1-L82-Fc;
   VH3-L83-VH4-L84-VL4-L85-VL3-L86-CL-L87-CH1-L88-Fc; or
   VH3-L83-VL4-L84-VH4-L85-VL3-L86-CL-L87-CH1-L88-Fc;
wherein:
VL1 is a first immunoglobulin light chain variable region that specifically binds to a tumor-associated antigen; VL2 is a second immunoglobulin light chain variable region that specifically binds to a tumor-associated antigen; VL3 is a third immunoglobulin light chain variable region that specifically binds to CD3; VL4 is a fourth immunoglobulin light chain variable region that specifically binds to a tumor-associated antigen; VH1 is a first immunoglobulin heavy chain variable region that specifically binds to a tumor-associated antigen; VH2 is a second immunoglobulin heavy chain variable region that specifically binds to a tumor-associated antigen; VH3 is a third immunoglobulin heavy chain variable region that specifically binds to CD3; VH4 is a fourth immunoglobulin heavy chain variable region that specifically binds to a tumor-associated antigen; Fc is a region comprising an immunoglobulin heavy chain constant region 2 (CH2), an immunoglobulin heavy chain constant region 3 (CH3), and optionally, an immunoglobulin hinge; CH1 is an immunoglobulin heavy chain constant region 1; CL is an immunoglobulin light chain constant region; and L1-L88 are amino acid linkers.
8. The antigen binding polypeptide complex according to clause 7, wherein the first polypeptide has a structure represented by: VL1-L1-VL2-L2-VH2-L3-VH1-L4-Fc; or VH1-L5-VH2-L6-VL2-L7-VL1-L8-Fc; wherein the second polypeptide has a structure represented by: VL3-L9-VL4-L10-VH4-L11-VH3-L12-Fc; or VH3-L13-VH4-L14-VL4-L15-VL3-L16-Fc.
9. An antigen binding polypeptide complex according to clause 7, wherein the first polypeptide has a structure represented by:
   VL1-L1-VL2-L2-VH2-L3-VH1-L4-CH1-L5-Fc;
   VH1-L6-VH2-L7-VL2-L8-VL1-L9-CH1-L10-Fc;
   VL1-L11-VL2-L12-VH2-L13-VH1-L14-CL-L15-Fc;
   VH1-L16-VH2-L17-VL2-L18-VL1-L19-CL-L20-Fc;
   VL1-L21-VL2-L22-VH2-L23-VH1-L24-CH1-L25-CL-L26-Fc;
   VH1-L27-VH2-L28-VL2-L29-VL1-L30-CH1-L31-CL-L32-Fc;
   VL1-L33-VL2-L34-VH2-L35-VH1-L36-CL-L37-CH1-L38-Fc; or
   VH1-L39-VH2-L40-VL2-L41-VL1-L42-CL-L43-CH1-L44-Fc;
wherein the second polypeptide has a structure represented by:
   VL3-L45-VL4-L46-VH4-L47-VH3-L48-CH1-L49-Fc;
   VH3-L50-VH4-L51-VL4-L52-VL3-L53-CH1-L54-Fc;
   VL3-L55-VL4-L56-VH4-L57-VH3-L58-CL-L59-Fc;
   VH3-L60-VH4-L61-VL4-L62-VL3-L63-CL-L64-Fc;
   VL3-L65-VL4-L66-VH4-L67-VH3-L68-CH1-L69-CL-L70-Fc;
   VH3-L71-VH4-L72-VL4-L73-VL3-L74-CH1-L75-CL-L76-Fc;
   VL3-L77-VL4-L78-VH4-L79-VH3-L80-CL-L81-CH1-L82-Fc; or
   VH3-L83-VH4-L84-VL4-L85-VL3-L86-CL-L87-CH1-L88-Fc.
10. The antigen binding polypeptide complex of any one of clauses 1 to 9, wherein one or more linkers L1-L88 has a length of from 0 amino acids to about 50 amino acids.
11. The antigen binding polypeptide complex of any one of clauses 1 to 10, wherein one or more linkers L1-L88 is non-immunogenic.
12. The antigen binding polypeptide complex of any one of clauses 1 to 11, wherein one or more linkers L1-L88 does not contain a consensus T cell epitope.
13. The antigen binding polypeptide complex of any one of clauses 1 to 12, wherein one or more linkers L1-L88 is a cleavable linker.
14. The antigen binding polypeptide complex of clause 13, wherein the cleavable linker is cleaved by a matrix metalloprotease (MMP), a type II transmembrane serine protease, or a MMP and a type II transmembrane serine protease.
15. The antigen binding polypeptide complex of clause 14, wherein the MMP is MMP1, MMP2, MMP7, MMP8, MMP9, MMP13, or a combination thereof.
16. The antigen binding polypeptide complex of clause 14, wherein the type II transmembrane serine protease is a matriptase, hepsin, or a combination thereof.
17. The antigen binding polypeptide complex of clause 16, wherein the matriptase is matriptase 1, matriptase 2, matriptase 3, or a combination thereof.
18. The antigen binding polypeptide complex of clause 13, wherein the cleavable linker is cleaved by urokinase or legumain.
19. The antigen binding polypeptide complex of clause 13, wherein the cleavable linker is GPAALV (SEQ ID NO: 22), GSGRKG (SEQ ID NO: 23), GPLGLTG (SEQ ID NO: 24), GPSGLVG (SEQ ID NO: 25), GLVGRKAG (SEQ ID NO: 26), GPAGLVG (SEQ ID NO: 27), GPAGLVSG (SEQ ID NO: 28), STRKAGG (SEQ ID NO: 29), ASTRKAG (SEQ ID NO: 30), or ASTRKAGG (SEQ ID NO: 31), or a sequence having at least 50%, at least 60%, at least 70%, at least 80%, at least 90%, or at least 95% identity to any one of SEQ ID NOs:22-31, 444, 567, 576 (GGS) and 607.
20. The antigen binding polypeptide complex of any one of clauses 1 to 19, wherein one or more of linkers L1-L88 comprise the amino acid sequence of g, a, gss, asg, ggssg (SEQ ID NO: 1), gssgs (SEQ ID NO: 2), gtvaa (SEQ ID NO: 3), asggs (SEQ ID NO: 4), astgg (SEQ ID NO: 5), ggsgs (SEQ ID NO: 6), asggsg (SEQ ID NO: 7), ggsgssg (SEQ ID NO: 8), ggsggssgss (SEQ ID NO: 9), sggsgssggs (SEQ ID NO: 10), ggsggsgsgggsasgsg (SEQ ID NO: 11), ggsggsgsggggsasgsg (SEQ ID NO: 12), gggssggggsggsgsggsgs (SEQ ID NO: 13), ggggsggsgsggggsasgsg (SEQ ID NO: 14), gggssggsgsggsgsggsgs (SEQ ID NO: 15), sggssggsgsggsgsggsgssg (SEQ ID NO: 16), gsgssggggsggsgsggsgssg (SEQ ID NO: 17), ggggsgsggsgggssggggsggggsggggsggggsggggs (SEQ ID NO: 18), ggggsggggsggggsggggsggggsggggsggggsggggs (SEQ ID NO: 19), ggggsgsggsgggssggggsggggsggggsggggsggggssss (SEQ ID NO: 20), or ggggsgsggsgggssggggsggggsggggsggggsggggssssgs (SEQ ID NO: 21), or any one of SEQ ID NOs: 444, 567, 576 (GGS) and 607, or a sequence having at least 50%, at least 60%, at least 70%, at least 80%, at least 90%, or at least 95% identity to any one of SEQ ID NOs:1-21, 444, 567, 576 (GGS) and 607.
21. The antigen binding polypeptide complex of any one of clauses 1 to 20, wherein one of VH1, VH2, VH3 or VH4 specifically binds to CD3.
22. The antigen binding polypeptide complex of any one of clauses 1 to 21, wherein one of VL1, VL2, VL3 or VL4 specifically binds to CD3.
23. The antigen binding polypeptide complex of any one of clauses 1 to 22, wherein VH1 and VL1, VH2 and VL2, VH3 and VL3, or VH4 and VL4 specifically binds to CD3.
24. The antigen binding polypeptide complex of any one of clauses 1 to 23,
wherein the VH1, VH2, VH3 or VH4 that specifically binds to CD3 comprises a CDR1 comprising an amino acid sequence having at least 90% identity, at least 95% identity, or 100% identity to any one of SEQ ID NOs:32, 40, 99, 433, 524, 667, 703, 739, 767, 803. 839, 851 and 859; a CDR2 comprising an amino acid sequence having at least 90% identity, at least 95% identity, or 100% identity to any one of SEQ ID NOs:33, 41, 100, 434, 525, 668, 704, 740, 768, 804, 840, 852 and 860; and a CDR3 comprising an amino acid sequence having at least 90% identity, at least 95% identity, or 100% identity to any one of SEQ ID NOs:34, 42, 101, 435, 526, 669, 705, 741, 769, 805, 841, 853 and 861; and
wherein the VL1, VL2, VL3 or VL4 that specifically binds to CD3 comprises a CDR1 comprising an amino acid sequence having at least 90% identity, at least 95% identity, or 100% identity to any one of SEQ ID NOs:35, 44, 96, 446, 456, 466, 476, 520, 663, 699, 735, 763, 799, 835, 855 and 863; a CDR2 comprising an amino acid sequence having at least 90% identity, at least 95% identity, or 100% identity to any one of SEQ ID NOs:36, 45, 97, 447 (DT), 457 (DT), 467 (DT), 477 (DT), 521, 664, 700, 736, 764, 800, 836, 856 and 864; and a CDR3 comprising an amino acid sequence having at least 90% identity, at least 95% identity, or 100% identity to any one of SEQ ID NOs:37, 46, 98, 448, 458, 468, 478, 522, 665, 701, 737, 765, 801, 837, 857 and 865.
25. The antigen binding polypeptide complex of clause 24,
wherein the VH1, VH2, VH3 or VH4 that specifically binds to CD3 comprises a CDR1 comprising an amino acid sequence having at least 90% identity to SEQ ID NO: 40; a CDR2 comprising an amino acid sequence having at least 90% identity to SEQ ID NO: 41; and a CDR3 comprising an amino acid sequence having at least 90% identity to SEQ ID NO: 42; and wherein the VL1, VL2, VL3 or VL4 that specifically binds to CD3 comprises a CDR1 comprising an amino acid sequence having at least 90% identity to SEQ ID NO: 44; a CDR2 comprising an amino acid sequence having at least 90% identity to SEQ ID NO: 45; and a CDR3 comprising an amino acid sequence having at least 90% identity to SEQ ID NO: 46;
optionally wherein the VH1, VH2, VH3 or VH4 that specifically binds to CD3 comprises a CDR1 comprising the amino acid sequence of SEQ ID NO: 40; a CDR2 comprising the amino acid sequence of SEQ ID NO: 41; and a CDR3 comprising the amino acid sequence of SEQ ID NO: 42; and wherein the VL1, VL2, VL3 or VL4 that specifically binds to CD3 comprises a CDR1 comprising the amino acid sequence of SEQ ID NO: 44; a CDR2 comprising the amino acid sequence of SEQ ID NO: 45; and a CDR3 comprising the amino acid sequence of SEQ ID NO: 46.
26. The antigen binding polypeptide complex of clause 24, wherein the VH1, VH2, VH3 or VH4 that specifically binds to CD3 comprises a CDR1 comprising an amino acid sequence having at least 90% identity to SEQ ID NO: 32; a CDR2 comprising an amino acid sequence having at least 90% identity to SEQ ID NO: 33; and a CDR3 comprising an amino acid sequence having at least 90% identity to SEQ ID NO: 34; and wherein the VL1, VL2, VL3 or VL4 that specifically binds to CD3 comprises a CDR1 comprising an amino acid sequence having at least 90% identity to SEQ ID NO: 35; a CDR2 comprising an amino acid sequence having at least 90% identity to SEQ ID NO: 36; and a CDR3 comprising an amino acid sequence having at least 90% identity to SEQ ID NO: 37;
optionally wherein the VH1, VH2, VH3 or VH4 that specifically binds to CD3 comprises a CDR1 comprising the amino acid sequence of SEQ ID NO: 32; a CDR2 comprising the amino acid sequence of SEQ ID NO: 33; and a CDR3 comprising the amino acid sequence of SEQ ID NO: 34; and wherein the VL1, VL2, VL3 or VL4 that specifically binds to CD3 comprises a CDR1 comprising the amino acid sequence of SEQ ID NO: 35 a CDR2 comprising the amino acid sequence of SEQ ID NO: 36; and a CDR3 comprising the amino acid sequence of SEQ ID NO: 37.
27. The antigen binding polypeptide complex of any one of clauses 1 to 24, wherein the VH1, VH2, VH3 or VH4 that specifically binds to CD3 comprises an amino acid sequence having at least 80%, at least 85%, at least 90%, at least 95%, or 100% identity to any one of SEQ ID NOs:38, 43, 102, 423, 432, 523, 666, 702, 738, 766, 802, 838, 850 and 858, and the VL1, VL2, VL3 or VL4 that specifically binds to CD3 comprises an amino acid sequence having at least 80%, at least 85%, at least 90%, at least 95%, or 100% identity to any one of SEQ ID NOs:39, 47, 103, 422, 445, 455, 465, 475, 519, 662, 698, 734, 762, 798, 834, 854 and 862.
28. The antigen binding polypeptide complex of clause 27, wherein the VH1, VH2, VH3 or VH4 that specifically binds to CD3 comprises an amino acid sequence having at least 80% identity to SEQ ID NO: 43, and the VL1, VL2, VL3 or VL4 that specifically binds to CD3 comprises an amino acid sequence having at least 80% identity to SEQ ID NO: 47; optionally wherein the VH1, VH2, VH3 or VH4 that specifically binds to CD3 comprises the amino acid sequence of SEQ ID NO: 43, and the VL1, VL2, VL3 or VL4 that specifically binds to CD3 comprises the amino acid sequence of SEQ ID NO: 47.
29. The antigen binding polypeptide complex of clause 27, wherein the VH1, VH2, VH3 or VH4 that specifically binds to CD3 comprises an amino acid sequence having at least 80% identity to SEQ ID NO: 38, and the VL1, VL2, VL3 or VL4 that specifically binds to CD3 comprises an amino acid sequence having at least 80% identity to SEQ ID NO: 39; optionally wherein the VH1, VH2, VH3 or VH4 that specifically binds to CD3 comprises the amino acid sequence of SEQ ID NO: 38, and the VL1, VL2, VL3 or VL4 that specifically binds to CD3 comprises the amino acid sequence of SEQ ID NO: 39.
30. The antigen binding polypeptide complex of any one of clauses 1 to 29, wherein one or more of VH1, VH2, VH3 or VH4 specifically binds to a tumor-associated antigen (TAA).
31. The antigen binding polypeptide complex of anyone of clauses 1 to 30, wherein one or more of VL1, VL2, VL3 or VL4 specifically binds to a TAA.
32. The antigen binding polypeptide complex of any one of clauses 1 to 31, wherein VH1 and VL1, VH2 and VL2, VH3 and VL3, and/or VH4 and VL4 specifically binds to a TAA.
33. The antigen binding polypeptide complex of any one of clauses 30 to 32, wherein the TAA is A2AR, APRIL, ATPDase, BAFF, BAFFR, BCMA, BlyS, BTK, BTLA, B7DC, B7H1, B7H2, B7H3, B7H4, B7H5, B7H6, B7H7, B7RP1, B7-4, C3, C5, CCL2, CCL3, CCL4, CCL5, CCL7, CCL8, CCL11, CCL15, CCL17, CCL19, CCL20, CCL21, CCL25, CCR3, CCR4, CD16A, CD19, CD20, CD24, CD27, CD28, CD30, CD38, CD39, CD40, CD40L, CD47, CD52, CD70, CD80, CD86, CD123, CD133, CD137, CD137L, CD160, CD272, CEACAM5, CLEC9, CLEC91, CRTH2, CSF-1, CSF-2, CSF-3, CXCL1, CXCL2, CXCL4, CXCL12, CXCL13, CXCR3, cMet, CTLA4, DLL3, DLL4, DNGR-1, E-cadherin, EGFR, ENTPD1, EpCAM, FCER1, FCER1A, FCER2, FGFR, FLAP, FOLH1, Gi24, GITR, GITRL, GPR5, GP100, GPRC5D, HER2, HER3, ICOSL, ICOS, HHLA2, HMGB1, HVEM, IDO, IFNa, IgE, IGF1R, IL2Rbeta, IL1, ILIA, IL1B, IL1F10, IL2, IL4, IL4Ra, IL5, IL5R, IL6, IL7, IL7Ra, IL8, IL9, IL9R, IL10, rhILlO, IL12, IL13, IL13Ra1, IL13Ra2, IL15, IL17, IL17Rb, IL18, IL22, IL23, IL25, IL7, IL33, IL35, ITGB4, ITK, KIR, LAG3, LAMP1, leptin, LPFS2, MHC class II, MUC-1, MUC-16, NCR3LG1, NKG2D, NKp46, NTPDase-1, OX40, OX40L, PD-1, PD-L1, PD-L2, PROM1, S152, SIRPalpha, SISP1, SLC, SPG64, ST2, STEAP1, STEAP2, Syk kinase, STEAP1, TROP2, TACI, TDO, TGFBETA, T14, TIGIT, TIM3, TLR, TLR2, TLR4, TLR5, TLR9, TMEF1, TNFa, TNFRSF7, Tp55, TREM1, TSLP, TSLPR, TWEAK, VEGF, VISTA, Vstm3, or WUCAM.
34. The antigen binding polypeptide complex of clause 33, wherein the TAA is cMet, Trop2, CD20, or CD19.
35. The antigen binding polypeptide complex of any one of clauses 30 to 34, wherein the one or more of VH1, VH2, VH3 or VH4 that specifically binds to a TAA comprises a CDR1 comprising an amino acid sequence having at least 90% identity, at least 95% identity, or 100% identity to any one of SEQ ID NOs:64, 72, 80, 88, 104, 112, 120, 128, 389, 409, 419, 429, 659, 695, 731, 397, 508, 540, 552, 757, 793, 829, 875, 500, 749, 785, 821, 649, 685, 775, 847, 625, 641, 677 and 713; a CDR2 comprising an amino acid sequence having at least 90% identity, at least 95% identity, or 100% identity to any one of SEQ ID NOs:65, 73, 81, 89, 105, 113, 121, 129, 390, 410, 420, 430, 660, 696, 732, 398, 509, 541, 553, 758, 794, 830, 876, 501, 750, 786, 822, 650, 686, 776, 848, 626, 642, 678 and 714; and a CDR3 comprising an amino acid sequence having at least 90% identity, at least 95% identity, or 100% identity to any one of SEQ ID NOs:66, 74, 82, 90, 106, 114, 122, 130, 391, 411, 421, 431, 661, 697, 733, 399, 510, 542, 554, 759, 795, 831, 877, 502, 751, 787, 823, 651, 687, 777, 849, 627, 643, 679 and 715; and/or
wherein the one or more of VL1, VL2, VL3 or VL4 that specifically binds to a TAA comprises a CDR1 comprising an amino acid sequence having at least 90% identity, at least 95% identity, or 100% identity to any one of SEQ ID NOs:67, 75, 83, 91, 107, 115, 123, 131, 385, 405, 415, 441, 452, 462, 472, 655, 691, 727, 393, 486, 504, 536, 548, 753, 789, 825, 871, 879, 490, 496, 745, 781, 817, 645, 681, 771, 843, 621, 637, 673 and 709; a CDR2 comprising an amino acid sequence having at least 90% identity, at least 95% identity, or 100% identity to any one of SEQ ID NOs:68, 76, 84, 92, 108, 116, 124, 132, 386 (YTS); 406 (YTS), 416 (YTS), 442 (YTS), 453 (YTS), 463 (YTS), 473 (YTS), 656, 692, 728, 394, 487 (AT), 505 (AT), 537, 549, 754, 790, 826, 872, 880, 491 (DA), 497 (DA), 746, 782, 818, 646, 682, 772, 844, 622, 638, 674 and 710; and a CDR3 comprising an amino acid sequence having at least 90% identity, at least 95% identity, or 100% identity to any one of SEQ ID NOs:69, 77, 85, 93, 109, 117, 125, 133, 387, 407, 417, 443, 454, 464, 474, 657, 693, 729, 395, 488, 506, 538, 550, 755, 791, 827, 873, 881, 492, 498, 747, 783, 819, 647, 683, 773, 845, 623, 639, 675 and 711.
36. The antigen binding polypeptide complex of clause 35, wherein the one or more of VH1, VH2, VH3 or VH4 specifically binds to CD38 and comprises a CDR1 comprising an amino acid sequence having at least 90% identity to SEQ ID NO: 64; a CDR2 comprising an amino acid sequence having at least 90% identity to SEQ ID NO: 65; and a CDR3 comprising an amino acid sequence having at least 90% identity to SEQ ID NO: 66; and wherein the one or more of VL1, VL2, VL3 or VL4 specifically binds to CD38 and comprises a CDR1 comprising an amino acid sequence having at least 90% identity to SEQ ID NO: 67; a CDR2 comprising an amino acid sequence having at least 90% identity to SEQ ID NO: 68; and a CDR3 comprising an amino acid sequence having at least 90% identity to SEQ ID NO: 69;
optionally wherein the VH1, VH2, VH3 or VH4 that specifically binds to CD38 comprises a CDR1 comprising the amino acid sequence of SEQ ID NO: 64; a CDR2 comprising the amino acid sequence of SEQ ID NO: 65; and a CDR3 comprising the amino acid sequence of SEQ ID NO: 66; and wherein the VL1, VL2, VL3 or VL4 that specifically binds to CD38 comprises a CDR1 comprising the amino acid sequence of SEQ ID NO: 67; a CDR2 comprising the amino acid sequence of SEQ ID NO: 68; and a CDR3 comprising the amino acid sequence of SEQ ID NO: 69.
37. The antigen binding polypeptide complex of clause 35, wherein the one or more of VH1, VH2, VH3 or VH4 specifically binds to cMet and comprises a CDR1 comprising an amino acid sequence having at least 90% identity to SEQ ID NO: 72; a CDR2 comprising an amino acid sequence having at least 90% identity to SEQ ID NO: 73; and a CDR3 comprising an amino acid sequence having at least 90% identity to SEQ ID NO: 74; and wherein the one or more of VL1, VL2, VL3 or VL4 specifically binds to cMet and comprises a CDR1 comprising an amino acid sequence having at least 90% identity to SEQ ID NO: 75; a CDR2 comprising an amino acid sequence having at least 90% identity to SEQ ID NO: 76; and a CDR3 comprising an amino acid sequence having at least 90% identity to SEQ ID NO: 77;
optionally wherein the VH1, VH2, VH3 or VH4 that specifically binds to cMet comprises a CDR1 comprising the amino acid sequence of SEQ ID NO: 72; a CDR2 comprising the amino acid sequence of SEQ ID NO: 73; and a CDR3 comprising the amino acid sequence of SEQ ID NO: 74; and wherein the VL1, VL2, VL3 or VL4 that specifically binds to cMet comprises a CDR1 comprising the amino acid sequence of SEQ ID NO: 75; a CDR2 comprising the amino acid sequence of SEQ ID NO: 76; and a CDR3 comprising the amino acid sequence of SEQ ID NO: 77.
38. The antigen binding polypeptide complex of clause 35, wherein the one or more of VH1, VH2, VH3 or VH4 specifically binds to Trop2 and comprises a CDR1 comprising an amino acid sequence having at least 90% identity to SEQ ID NO: 80; a CDR2 comprising an amino acid sequence having at least 90% identity to SEQ ID NO: 81; and a CDR3 comprising an amino acid sequence having at least 90% identity to SEQ ID NO: 82; and wherein the one or more of VL1, VL2, VL3 or VL4 specifically binds to Trop2 and comprises a CDR1 comprising an amino acid sequence having at least 90% identity to SEQ ID NO: 83; a CDR2 comprising an amino acid sequence having at least 90% identity to SEQ ID NO: 84; and a CDR3 comprising an amino acid sequence having at least 90% identity to SEQ ID NO: 85;
optionally wherein the VH1, VH2, VH3 or VH4 that specifically binds to Trop2 comprises a CDR1 comprising the amino acid sequence of SEQ ID NO: 80; a CDR2 comprising the amino acid sequence of SEQ ID NO: 81; and a CDR3 comprising the amino acid sequence of SEQ ID NO: 82; and wherein the VL1, VL2, VL3 or VL4 that specifically binds to Trop2 comprises a CDR1 comprising the amino acid sequence of SEQ ID NO: 83; a CDR2 comprising the amino acid sequence of SEQ ID NO: 84; and a CDR3 comprising the amino acid sequence of SEQ ID NO: 85.
39. The antigen binding polypeptide complex of clause 35, wherein the one or more of VH1, VH2, VH3 or VH4 specifically binds to Trop2 and comprises a CDR1 comprising an amino acid sequence having at least 90% identity to SEQ ID NO: 88; a CDR2 comprising an amino acid sequence having at least 90% identity to SEQ ID NO: 89; and a CDR3 comprising an amino acid sequence having at least 90% identity to SEQ ID NO: 90; and wherein the one or more of VL1, VL2, VL3 or VL4 specifically binds to Trop2 and comprises a CDR1 comprising an amino acid sequence having at least 90% identity to SEQ ID NO: 91; a CDR2 comprising an amino acid sequence having at least 90% identity to SEQ ID NO: 92; and a CDR3 comprising an amino acid sequence having at least 90% identity to SEQ ID NO: 93;
optionally wherein the VH1, VH2, VH3 or VH4 that specifically binds to Trop2 comprises a CDR1 comprising the amino acid sequence of SEQ ID NO: 88; a CDR2 comprising the amino acid sequence of SEQ ID NO: 89; and a CDR3 comprising the amino acid sequence of SEQ ID NO: 90; and wherein the VL1, VL2, VL3 or VL4 that specifically binds to Trop2 comprises a CDR1 comprising the amino acid sequence of SEQ ID NO: 91; a CDR2 comprising the amino acid sequence of SEQ ID NO: 92; and a CDR3 comprising the amino acid sequence of SEQ ID NO: 93.
40. The antigen binding polypeptide complex of clause 35, wherein the one or more of VH1, VH2, VH3 or VH4 that specifically binds to CD3 and comprises a CDR1 comprising an amino acid sequence having at least 90% identity to SEQ ID NO: 96; a CDR2 comprising an amino acid sequence having at least 90% identity to SEQ ID NO: 97; and a CDR3 comprising an amino acid sequence having at least 90% identity to SEQ ID NO: 98; and wherein the one or more of VL1, VL2, VL3 or VL4 specifically binds to CD3 and comprises a CDR1 comprising an amino acid sequence having at least 90% identity to SEQ ID NO: 99; a CDR2 comprising an amino acid sequence having at least 90% identity to SEQ ID NO: 100; and a CDR3 comprising an amino acid sequence having at least 90% identity to SEQ ID NO: 101;
optionally wherein the VH1, VH2, VH3 or VH4 that specifically binds to CD3 comprises a CDR1 comprising the amino acid sequence of SEQ ID NO: 96; a CDR2 comprising the amino acid sequence of SEQ ID NO: 97; and a CDR3 comprising the amino acid sequence of SEQ ID NO: 98; and wherein the VL1, VL2, VL3 or VL4 that specifically binds to CD3 comprises a CDR1 comprising the amino acid sequence of SEQ ID NO: 99; a CDR2 comprising the amino acid sequence of SEQ ID NO: 100; and a CDR3 comprising the amino acid sequence of SEQ ID NO: 101.
41. The antigen binding polypeptide complex of clause 35, wherein the one or more of VH1, VH2, VH3 or VH4 specifically binds to CD20 and comprises a CDR1 comprising an amino acid sequence having at least 90% identity to SEQ ID NO: 104; a CDR2 comprising an amino acid sequence having at least 90% identity to SEQ ID NO: 105; and a CDR3 comprising an amino acid sequence having at least 90% identity to SEQ ID NO: 106; and wherein the one or more of VL1, VL2, VL3 or VL4 specifically binds to CD20 and comprises a CDR1 comprising an amino acid sequence having at least 90% identity to SEQ ID NO: 107; a CDR2 comprising an amino acid sequence having at least 90% identity to SEQ ID NO: 108; and a CDR3 comprising an amino acid sequence having at least 90% identity to SEQ ID NO: 109;
optionally wherein the VH1, VH2, VH3 or VH4 that specifically binds to CD20 comprises a CDR1 comprising the amino acid sequence of SEQ ID NO: 104; a CDR2 comprising the amino acid sequence of SEQ ID NO: 105; and a CDR3 comprising the amino acid sequence of SEQ ID NO: 106; and wherein the VL1, VL2, VL3 or VL4 that specifically binds to CD20 comprises a CDR1 comprising the amino acid sequence of SEQ ID NO: 107; a CDR2 comprising the amino acid sequence of SEQ ID NO: 108; and a CDR3 comprising the amino acid sequence of SEQ ID NO: 109.
42. The antigen binding polypeptide complex of clause 35, wherein the one or more of VH1, VH2, VH3 or VH4 specifically binds to CD20 and comprises a CDR1 comprising an amino acid sequence having at least 90% identity to SEQ ID NO: 112; a CDR2 comprising an amino acid sequence having at least 90% identity to SEQ ID NO: 113; and a CDR3 comprising an amino acid sequence having at least 90% identity to SEQ ID NO: 114; and wherein the one or more of VL1, VL2, VL3 or VL4 specifically binds to CD20 and comprises a CDR1 comprising an amino acid sequence having at least 90% identity to SEQ ID NO: 115; a CDR2 comprising an amino acid sequence having at least 90% identity to SEQ ID NO: 116; and a CDR3 comprising an amino acid sequence having at least 90% identity to SEQ ID NO: 117;
optionally wherein the VH1, VH2, VH3 or VH4 that specifically binds to CD20 comprises a CDR1 comprising the amino acid sequence of SEQ ID NO: 112; a CDR2 comprising the amino acid sequence of SEQ ID NO: 113; and a CDR3 comprising the amino acid sequence of SEQ ID NO: 114; and wherein the VL1, VL2, VL3 or VL4 that specifically binds to CD20 comprises a CDR1 comprising the amino acid sequence of SEQ ID NO: 115; a CDR2 comprising the amino acid sequence of SEQ ID NO: 116; and a CDR3 comprising the amino acid sequence of SEQ ID NO: 117.
43. The antigen binding polypeptide complex of clause 35, wherein the one or more of VH1, VH2, VH3 or VH4 specifically binds to CD19 and comprises a CDR1 comprising an amino acid sequence having at least 90% identity to SEQ ID NO: 120; a CDR2 comprising an amino acid sequence having at least 90% identity to SEQ ID NO: 121; and a CDR3 comprising an amino acid sequence having at least 90% identity to SEQ ID NO: 122; and wherein the one or more of VL1, VL2, VL3 or VL4 specifically binds to CD19 and comprises a CDR1 comprising an amino acid sequence having at least 90% identity to SEQ ID NO: 123; a CDR2 comprising an amino acid sequence having at least 90% identity to SEQ ID NO: 124; and a CDR3 comprising an amino acid sequence having at least 90% identity to SEQ ID NO: 125; optionally
wherein the VH1, VH2, VH3 or VH4 that specifically binds to CD19 comprises a CDR1 comprising the amino acid sequence of SEQ ID NO: 120; a CDR2 comprising the amino acid sequence of SEQ ID NO: 121; and a CDR3 comprising the amino acid sequence of SEQ ID NO: 122; and wherein the VL1, VL2, VL3 or VL4 that specifically binds to CD19 comprises a CDR1 comprising the amino acid sequence of SEQ ID NO: 123; a CDR2 comprising the amino acid sequence of SEQ ID NO: 124; and a CDR3 comprising the amino acid sequence of SEQ ID NO: 125.
44. The antigen binding polypeptide complex of clause 35, wherein the one or more of VH1, VH2, VH3 or VH4 specifically binds to CD19 and comprises a CDR1 comprising an amino acid sequence having at least 90% identity to SEQ ID NO: 128; a CDR2 comprising an amino acid sequence having at least 90% identity to SEQ ID NO: 129; and a CDR3 comprising an amino acid sequence having at least 90% identity to SEQ ID NO: 130; and wherein the one or more of VL1, VL2, VL3 or VL4 specifically binds to CD19 and comprises a CDR1 comprising an amino acid sequence having at least 90% identity to SEQ ID NO: 131; a CDR2 comprising an amino acid sequence having at least 90% identity to SEQ ID NO: 132; and a CDR3 comprising an amino acid sequence having at least 90% identity to SEQ ID NO: 133; optionally
wherein the VH1, VH2, VH3 or VH4 that specifically binds to CD19 comprises a CDR1 comprising the amino acid sequence of SEQ ID NO: 128; a CDR2 comprising the amino acid sequence of SEQ ID NO: 129; and a CDR3 comprising the amino acid sequence of SEQ ID NO: 130; and wherein the VL1, VL2, VL3 or VL4 that specifically binds to CD19 comprises a CDR1 comprising the amino acid sequence of SEQ ID NO: 131; a CDR2 comprising the amino acid sequence of SEQ ID NO: 132; and a CDR3 comprising the amino acid sequence of SEQ ID NO: 133.
45. The antigen binding polypeptide complex of any one of clauses 30 to 44, wherein the one or more of VH1, VH2, VH3 or VH4 that specifically binds to a TAA comprises an amino acid sequence having at least 80%, at least 85%, at least 90%, at least 95%, or 100% identity to any one of SEQ ID NOs:70, 78, 86, 94, 102, 110, 118, 126, 134, 388, 408, 418, 428, 584, 658, 694, 730, 396, 403, 507, 539, 551, 756, 792, 828, 866, 874, 499, 544, 556, 748, 784, 820, 883, 528, 648, 684, 774, 846, 624, 640, 676 and 712; and the one or more of VL1, VL2, VL3 or VL4 that specifically binds to a TAA comprises an amino acid sequence having at least 80%, at least 85%, at least 90%, at least 95%, or 100% identity to any one of SEQ ID NOs:71, 79, 87, 95, 103, 111, 119, 127, 135, 384, 404, 414, 440, 451, 461, 471, 583, 654, 690, 726, 392, 402, 485, 503, 535, 547, 752, 788, 824, 870, 878, 489, 495, 543, 555, 744, 780, 882, 816, 527, 644, 680, 770, 842, 620, 636, 672 and 708.
46. The antigen binding polypeptide complex of clause 45, wherein the one or more of VH1, VH2, VH3 or VH4 specifically binds to CD38 and comprises a sequence having at least 80% identity to SEQ ID NO: 70 and wherein the one or more of VL1, VL2, VL3 or VL4 specifically binds to CD38 and comprises a sequence having at least 80% identity to SEQ ID NO: 71; optionally wherein the VH1, VH2, VH3 or VH4 that specifically binds to CD38 comprises the amino acid sequence of SEQ ID NO: 70; and wherein the VL1, VL2, VL3 or VL4 that specifically binds to CD38 comprises the amino acid sequence of SEQ ID NO: 71.
47. The antigen binding polypeptide complex of clause 45, wherein the one or more of VH1, VH2, VH3 or VH4 specifically binds to cMet and comprises a sequence having at least 80% identity to SEQ ID NO: 78 and wherein the one or more of VL1, VL2, VL3 or VL4 specifically binds to cMet and comprises a sequence having at least 80% identity to SEQ ID NO: 79; optionally wherein the VH1, VH2, VH3 or VH4 that specifically binds to cMet comprises the amino acid sequence of SEQ ID NO: 78; and wherein the VL1, VL2, VL3 or VL4 that specifically binds to cMet comprises the amino acid sequence of SEQ ID NO: 79.
48. The antigen binding polypeptide complex of clause 45, wherein the one or more of VH1, VH2, VH3 or VH4 specifically binds to Trop2 and comprises a sequence having at least 80% identity to SEQ ID NO: 86; and wherein the one or more of VL1, VL2, VL3 or VL4 specifically binds to Trop2 and comprises a sequence having at least 80% identity to SEQ ID NO: 87; optionally wherein the VH1, VH2, VH3 or VH4 that specifically binds to Trop2 comprises the amino acid sequence of SEQ ID NO: 86; and wherein the VL1, VL2, VL3 or VL4 that specifically binds to Trop2 comprises the amino acid sequence of SEQ ID NO: 87.
49. The antigen binding polypeptide complex of clause 45, wherein the one or more of VH1, VH2, VH3 or VH4 specifically binds to Trop2 and comprises a sequence having at least 80% identity to SEQ ID NO: 94; and wherein the one or more of VL1, VL2, VL3 or VL4 specifically binds to Trop2 and comprises a sequence having at least 80% identity to SEQ ID NO: 95; optionally wherein the VH1, VH2, VH3 or VH4 that specifically binds to Trop2 comprises the amino acid sequence of SEQ ID NO: 94; and wherein the VL1, VL2, VL3 or VL4 that specifically binds to Trop2 comprises the amino acid sequence of SEQ ID NO: 95.
50. The antigen binding polypeptide complex of clause 45, wherein the one or more of VH1, VH2, VH3 or VH4 specifically binds to CD3 and comprises a sequence having at least 80% identity to SEQ ID NO: 102; and wherein the one or more of VL1, VL2, VL3 or VL4 specifically binds to CD3 and comprises a sequence having at least 80% identity to SEQ ID NO: 103; optionally wherein the VH1, VH2, VH3 or VH4 that specifically binds to CD3 comprises the amino acid sequence of SEQ ID NO: 102; and wherein the VL1, VL2, VL3 or VL4 that specifically binds to CD3 comprises the amino acid sequence of SEQ ID NO: 103.
51. The antigen binding polypeptide complex of clause 45, wherein the one or more of VH1, VH2, VH3 or VH4 specifically binds to CD20 and comprises a sequence having at least 80% identity to SEQ ID NO: 110; and wherein the one or more of VL1, VL2, VL3 or VL4 specifically binds to CD20 and comprises a sequence having at least 80% identity to SEQ ID NO: 111; optionally wherein the VH1, VH2, VH3 or VH4 that specifically binds to CD20 comprises the amino acid sequence of SEQ ID NO: 110; and wherein the VL1, VL2, VL3 or VL4 that specifically binds to CD20 comprises the amino acid sequence of SEQ ID NO: 111.
52. The antigen binding polypeptide complex of clause 45, wherein the one or more of VH1, VH2, VH3 or VH4 specifically binds to CD20 and comprises a sequence having at least 80% identity to SEQ ID NO: 110; and wherein the one or more of VL1, VL2, VL3 or VL4 specifically binds to CD20 and comprises a sequence having at least 80% identity to SEQ ID NO: 119; optionally wherein the VH1, VH2, VH3 or VH4 that specifically binds to CD20 comprises the amino acid sequence of SEQ ID NO: 110; and wherein the VL1, VL2, VL3 or VL4 that specifically binds to CD20 comprises the amino acid sequence of SEQ ID NO: 119.
53. The antigen binding polypeptide complex of clause 45, wherein the one or more of VH1, VH2, VH3 or VH4 specifically binds to CD20 and comprises a sequence having at least 80% identity to SEQ ID NO: 118; and wherein the one or more of VL1, VL2, VL3 or VL4 specifically binds to CD20 and comprises a sequence having at least 80% identity to SEQ ID NO: 111; optionally wherein the VH1, VH2, VH3 or VH4 that specifically binds to CD20 comprises the amino acid sequence of SEQ ID NO: 118; and wherein the VL1, VL2, VL3 or VL4 that specifically binds to CD20 comprises the amino acid sequence of SEQ ID NO: 111.
54. The antigen binding polypeptide complex of clause 45, wherein the one or more of VH1, VH2, VH3 or VH4 specifically binds to CD20 and comprises a sequence having at least 80% identity to SEQ ID NO: 118; and wherein the one or more of VL1, VL2, VL3 or VL4 specifically binds to CD20 and comprises a sequence having at least 80% identity to SEQ ID NO: 119; optionally wherein the VH1, VH2, VH3 or VH4 that specifically binds to CD20 comprises the amino acid sequence of SEQ ID NO: 118; and wherein the VL1, VL2, VL3 or VL4 that specifically binds to CD20 comprises the amino acid sequence of SEQ ID NO: 119.
55. The antigen binding polypeptide complex of clause 45, wherein the one or more of VH1, VH2, VH3 or VH4 specifically binds to CD19 and comprises a sequence having at least 80% identity to SEQ ID NO: 126; and wherein the one or more of VL1, VL2, VL3 or VL4 specifically binds to CD19 and comprises a sequence having at least 80% identity to SEQ ID NO: 127; optionally wherein the VH1, VH2, VH3 or VH4 that specifically binds to CD19 comprises the amino acid sequence of SEQ ID NO: 126; and wherein the VL1, VL2, VL3 or VL4 that specifically binds to CD19 comprises the amino acid sequence of SEQ ID NO: 127; or
wherein the one or more of VH1, VH2, VH3 or VH4 specifically binds to CD19 and comprises a sequence having at least 80% identity to SEQ ID NO:883; and wherein the one or more of VL1, VL2, VL3 or VL4 specifically binds to CD19 and comprises a sequence having at least 80% identity to SEQ ID NO:882; optionally wherein the VH1, VH2, VH3 or VH4 that specifically binds to CD19 comprises the amino acid sequence of SEQ ID NO:883; and wherein the VL1, VL2, VL3 or VL4 that specifically binds to CD19 comprises the amino acid sequence of SEQ ID NO:882.
56. The antigen binding polypeptide complex of clause 45, wherein the one or more of VH1, VH2, VH3 or VH4 specifically binds to CD19 and comprises a sequence having at least 80% identity to SEQ ID NO: 134; and wherein the one or more of VL1, VL2, VL3 or VL4 specifically binds to CD19 and comprises a sequence having at least 80% identity to SEQ ID NO: 135; optionally wherein the VH1, VH2, VH3 or VH4 that specifically binds to CD19 comprises the amino acid sequence of SEQ ID NO: 134; and wherein the VL1, VL2, VL3 or VL4 that specifically binds to CD19 comprises the amino acid sequence of SEQ ID NO: 135.
57. The antigen binding polypeptide complex of any one of clauses 1 to 56, wherein one or more of VH1, VH2, VH3 or VH4 specifically binds to an immune stimulating receptor.
58. The antigen binding polypeptide complex of any one of clauses 1 to 57, wherein one or more of VL1, VL2, VL3 or VL4 specifically binds to an immune stimulating receptor.
59. The antigen binding polypeptide complex of any one of clauses 1 to 58, wherein VH1 and VL1, VH2 and VL2, VH3 and VL3, and/or VH4 and VL4 specifically binds to an immune stimulating receptor.
60. The antigen binding polypeptide complex of any one of clauses 57 to 59, wherein the immune stimulating receptor is CD28.
61. The antigen binding polypeptide complex of any one of clauses 57 to 60, wherein the VH1, VH2, VH3 or VH4 that specifically binds to an immune stimulating receptor comprises a CDR1 comprising an amino acid sequence having at least 90% identity, at least 95% identity, or 100% identity to any one of SEQ ID NOs:48, 56, 649, 685, 775 and 847; a CDR2 comprising an amino acid sequence having at least 90% identity, at least 95% identity, or 100% identity to any one of SEQ ID NOs:49, 57, 650, 686, 776 and 848; and a CDR3 comprising an amino acid sequence having at least 90% identity, at least 95% identity, or 100% identity to any one of SEQ ID NOs:50, 58, 651, 687, 777 and 849; and
wherein the VL1, VL2, VL3 or VL4 that specifically binds to an immune stimulating receptor comprises a CDR1 comprising an amino acid sequence having at least 90% identity, at least 95% identity, or 100% identity to any one of SEQ ID NOs:51, 59, 645, 681, 771 and 843; a CDR2 comprising an amino acid sequence having at least 90% identity, at least 95% identity, or 100% identity to any one of SEQ ID NOs:52, 60, 646, 682, 772 and 844; and a CDR3 comprising an amino acid sequence having at least 90% identity, at least 95% identity, or 100% identity to any one of SEQ ID NOs:53, 61, 647, 683, 773 and 845.
62. The antigen binding polypeptide complex of clause 61, wherein the VH1, VH2, VH3 or VH4 that specifically binds to an immune stimulating receptor comprises a CDR1 comprising an amino acid sequence having at least 90% identity to SEQ ID NO:48; a CDR2 comprising an amino acid sequence having at least 90% identity to SEQ ID NO:49; and a CDR3 comprising an amino acid sequence having at least 90% identity to SEQ ID NO:50; and wherein the VL1, VL2, VL3 or VL4 that specifically binds to an immune stimulating receptor comprises a CDR1 comprising an amino acid sequence having at least at least 90% identity to SEQ ID NO:51; a CDR2 comprising an amino acid sequence having at least 90% identity to SEQ ID NO:52; and a CDR3 comprising an amino acid sequence having at least 90% identity to SEQ ID NO:53; optionally
wherein the VH1, VH2, VH3 or VH4 that specifically binds to an immune stimulating receptor comprises a CDR1 comprising the amino acid of SEQ ID NO:48; a CDR2 comprising the amino acid of SEQ ID NO:49; and a CDR3 comprising the amino acid of SEQ ID NO:50; and wherein the VL1, VL2, VL3 or VL4 that specifically binds to an immune stimulating receptor comprises a CDR1 comprising the amino acid of SEQ ID NO:51; a CDR2 comprising the amino acid of SEQ ID NO:52; and a CDR3 comprising the amino acid of SEQ ID NO:53.
63. The antigen binding polypeptide complex of clause 61, wherein the VH1, VH2, VH3 or VH4 that specifically binds to an immune stimulating receptor comprises a CDR1 comprising an amino acid sequence having at least 90% identity to SEQ ID NO:56; a CDR2 comprising an amino acid sequence having at least 90% identity to SEQ ID NO:57; and a CDR3 comprising an amino acid sequence having at least 90% identity to SEQ ID NO:58; and wherein the VL1, VL2, VL3 or VL4 that specifically binds to an immune stimulating receptor comprises a CDR1 comprising an amino acid sequence having at least at least 90% identity to SEQ ID NO:59; a CDR2 comprising an amino acid sequence having at least 90% identity to SEQ ID NO:60; and a CDR3 comprising an amino acid sequence having at least 90% identity to SEQ ID NO:61; optionally
wherein the VH1, VH2, VH3 or VH4 that specifically binds to an immune stimulating receptor comprises a CDR1 comprising the amino acid of SEQ ID NO:56; a CDR2 comprising the amino acid of SEQ ID NO:57; and a CDR3 comprising the amino acid of SEQ ID NO:58; and wherein the VL1, VL2, VL3 or VL4 that specifically binds to an immune stimulating receptor comprises a CDR1 comprising the amino acid of SEQ ID NO:59; a CDR2 comprising the amino acid of SEQ ID NO:60; and a CDR3 comprising the amino acid of SEQ ID NO:61.
64. The antigen binding polypeptide complex of any one of clauses 57 to 63, wherein the VH1, VH2, VH3 or VH4 that specifically binds to an immune stimulating receptor comprises an amino acid sequence having at least 80%, at least 85%, at least 90%, at least 95%, or 100% identity to any one of SEQ ID NOs:54, 62, 528, 648, 684, 774 and 846, and the VL1, VL2, VL3 or VL4 that specifically binds to an immune stimulating receptor comprises an amino acid sequence having at least 80%, at least 85%, at least 90%, at least 95%, or 100% identity to any one of SEQ ID NOs:55, 63, 527, 644, 680, 770 and 842.
65. The antigen binding polypeptide complex of clause 64, wherein the VH1, VH2, VH3 or VH4 that specifically binds to an immune stimulating receptor comprises an amino acid sequence having at least 80% identity to SEQ ID NO:54, and the VL1, VL2, VL3 or VL4 that specifically binds to an immune stimulating receptor comprises an amino acid sequence having at least 80% identity to SEQ ID NO:55; optionally wherein the VH1, VH2, VH3 or VH4 that specifically binds to an immune stimulating receptor comprises the amino acid sequence of SEQ ID NO:54, and the VL1, VL2, VL3 or VL4 that specifically binds to an immune stimulating receptor comprising the amino acid of SEQ ID NO:55.
66. The antigen binding polypeptide complex of clause 64, wherein the VH1, VH2, VH3 or VH4 that specifically binds to an immune stimulating receptor comprises an amino acid sequence having at least 80% identity to SEQ ID NO:62, and the VL1, VL2, VL3 or VL4 that specifically binds to an immune stimulating receptor comprises an amino acid sequence having at least 80% identity to SEQ ID NO:63; optionally wherein the VH1, VH2, VH3 or VH4 that specifically binds to an immune stimulating receptor comprises the amino acid sequence of SEQ ID NO:62, and the VL1, VL2, VL3 or VL4 that specifically binds to an immune stimulating receptor comprising the amino acid of SEQ ID NO:63.
67. The antigen binding polypeptide complex of any one of clauses 1 to 66, wherein L5, L8, or L5 and L8 are cleavable linkers.
68. The antigen binding polypeptide complex of clause 67, wherein VH3 and VL3 specifically bind to CD3.
69. The antigen binding polypeptide complex of clause 67 or 68, wherein VH1 and VL1 and/or VH2 and VL2 specifically bind to a TAA; optionally wherein the TAA is selected from CD19 and CD20; or wherein the TAA is selected from cMet and Trop2.
70. The antigen binding polypeptide complex of any one of any one of clauses 67 to 69, wherein VH1 and VL1 or VH2 and VL2 specifically bind to CD28.
71. The antigen binding polypeptide complex of any one of clauses 1 to 70, wherein L4, L7, or L4 and L7 are cleavable linkers.
72. The antigen binding polypeptide complex of clause 71, wherein VH3 and VL3 specifically bind to CD3.
73. The antigen binding polypeptide complex of clause 71 or 72, wherein VH1 and VL1 and/or VH2 and VL2 specifically bind to a TAA; optionally wherein the TAA is selected from CD19 and CD20; or wherein the TAA is selected from cMet and Trop2.
74. The antigen binding polypeptide complex of any one of clauses 71 to 73, wherein VH1 and VL1 or VH2 and VL2 specifically bind to CD28.
75. The antigen binding polypeptide complex of any one of clauses 1 to 74, wherein L9, L12, or L9 and L12 are cleavable linkers.
76. The antigen binding polypeptide complex of clause 75, wherein VH4 and VL4 specifically bind to CD3.
77. The antigen binding polypeptide complex of clause 75 or clause 76, wherein one or more of VH1 and VL1, VH2 and VL2, and VH3 and VL3 specifically bind to a TAA.
78. The antigen binding polypeptide complex of any one of clauses 75 to 77, wherein VH1 and VL1 specifically bind to a TAA, VH2 and VL2 specifically bind to a TAA, and VH3 and VL3 specifically bind to CD28.
79. The antigen binding polypeptide complex of any one of clauses 75 to 77, wherein VH1 and VL1 specifically bind to CD28, VH2 and VL2 specifically bind to a TAA, and VH3 and VL3 specifically bind to a TAA.
80. The antigen binding polypeptide complex of any one of clauses 75 to 77, wherein VH1 and VL1 specifically bind to cMet, VH2 and VL2 specifically bind to Trop2, and VH3 and VL3 specifically bind to CD28.
81. The antigen binding polypeptide complex of any one of clauses 75 to 77, wherein VH1 and VL1 specifically bind to Trop2, VH2 and VL2 specifically bind to cMet, and VH3 and VL3 specifically bind to CD28.
82. The antigen binding polypeptide complex of any one of clauses 75 to 77, wherein VH1 and VL1 specifically bind to cMet, VH2 and VL2 specifically bind to CD28, and VH3 and VL3 specifically bind to Trop2.
83. The antigen binding polypeptide complex of any one of clauses 75 to 77, wherein VH1 and VL1 specifically bind to CD28, VH2 and VL2 specifically bind to cMet, and VH3 and VL3 specifically bind to Trop2.
84. The antigen binding polypeptide complex of any one of clauses 75 to 77, wherein VH1 and VL1 specifically bind to Trop2, VH2 and VL2 specifically bind to CD28, and VH3 and VL3 specifically bind to cMet.
85. The antigen binding polypeptide complex of any one of clauses 75 to 77, wherein VH1 and VL1 specifically bind to CD28, VH2 and VL2 specifically bind to Trop2, and VH3 and VL3 specifically bind to cMet.
86. The antigen binding polypeptide complex of any one of clauses 75 to 77, wherein VH1 and VL1 specifically bind to CD19, VH2 and VL2 specifically bind to CD20, and VH3 and VL3 specifically bind to CD28.
87. The antigen binding polypeptide complex of any one of clauses 75 to 77, wherein VH1 and VL1 specifically bind to CD20, VH2 and VL2 specifically bind to CD19, and VH3 and VL3 specifically bind to CD28.
88. The antigen binding polypeptide complex of any one of clauses 75 to 77, wherein VH1 and VL1 specifically bind to CD19, VH2 and VL2 specifically bind to CD28, and VH3 and VL3 specifically bind to CD20.
89. The antigen binding polypeptide complex of any one of clauses 75 to 77, wherein VH1 and VL1 specifically bind to CD20, VH2 and VL2 specifically bind to CD28, and VH3 and VL3 specifically bind to CD19.
90. The antigen binding polypeptide complex of any one of clauses 75 to 77, wherein VH1 and VL1 specifically bind to CD28, VH2 and VL2 specifically bind to CD19, and VH3 and VL3 specifically bind to CD20.
91. The antigen binding polypeptide complex of any one of clauses 75 to 77, wherein VH1 and VL1 specifically bind to CD28, VH2 and VL2 specifically bind to CD20, and VH3 and VL3 specifically bind to CD19.
92. The antigen binding polypeptide complex of any one of clauses 1 to 91, wherein L1, L3, or L1 and L3 are cleavable linkers, or L5, L7, or L5 and L7 are cleavable linkers.
93. The antigen binding polypeptide complex of any one of clauses 1 to 91, wherein L9, L11, or L9 and L11 are cleavable linkers, or L13, L15, or L13 and L15 are cleavable linkers.
94. The antigen binding polypeptide complex of clause 92 or clause 93, wherein VH1 and VL1, VH2 and VL2, VH3 and VL3, or VH4 and VL4 specifically bind to CD3.
95. The antigen binding polypeptide complex of any one of clauses 92 to 94, wherein one or more of VH1 and VL1, VH2 and VL2, VH3 and VL3, and VH4 and VL4 specifically bind to a TAA; optionally wherein the TAA is selected from CD19 and CD20; or wherein the TAA is selected from cMet and Trop2.
96. The antigen binding polypeptide complex of any one of clauses 92 to 95, wherein VH1 and VL1, VH2 and VL2, VH3 and VL3, and/or VH4 and VL4 specifically bind to CD28.
97. The antigen binding polypeptide complex of any one of clauses 1 to 96, wherein the antigen binding polypeptide complex is an antibody or antigen binding fragment thereof.
98. The antigen binding polypeptide complex of any one of clauses 1 to 97, wherein the immunoglobulin hinge comprises an upper hinge region, a middle hinge region, a lower hinge region, or a combination thereof.
99. The antigen binding polypeptide complex of any one of clauses 1 to 98, wherein the Fc region comprises at least one knob-into-hole modification.
100. The antigen binding polypeptide complex of clause 99, wherein the antigen binding polypeptide complex is an IgG1 or IgG4 antibody and the knob-into-hole modification comprises:
(i) knob substitutions of S354C and T366W and hole substitutions of Y349C, T366S, L368A and Y407V;
(ii) hole substitutions of L234A, L235A and P329A;
(iii) hole substitutions of L234A and L235A;
(iv) hole substitutions of M428L and N433S;
(v) hole substitutions of M252Y, S254T and T256E; or
(vi) a combination thereof;
   based on the EU numbering scheme.
101. An antibody or antigen binding fragment thereof comprising the antigen binding polypeptide complex of any one of clauses 1 to 100.
102. A pharmaceutical composition comprising the antigen binding polypeptide complex of any one of clauses 1 to 100, or the antibody or antigen binding fragment thereof of clause 101, and a pharmaceutically acceptable carrier.
103. A kit comprising an antigen binding polypeptide complex of any one of clauses 1 to 100, an antibody or antigen binding fragment thereof of clause 101, or the pharmaceutical composition of clause 102, and instructions for use.
104. An antigen binding polypeptide complex of any one of clauses 1 to 100, the antibody or antigen binding fragment thereof of clause 101, or the pharmaceutical composition of clause 102 for use in treating cancer in a subject in need thereof.
105. The antigen binding polypeptide complex, antibody or antigen binding fragment thereof or the pharmaceutical composition for use according to clause 104, wherein the cancer is breast cancer, lung cancer, gastric cancer, or prostate cancer, cervical cancer, urothelial cancer, or pancreatic cancer.
106. The antigen binding polypeptide complex, antibody or antigen binding fragment thereof or the pharmaceutical composition for use according to clause 104, wherein the cancer is a hematological cancer.
107. The antigen binding polypeptide complex, antibody or antigen binding fragment thereof or the pharmaceutical composition for use according to clause 104, wherein the cancer is a solid cancer.
108. The antigen binding polypeptide complex, antibody or antigen binding fragment thereof or the pharmaceutical composition for use according to clause 106, wherein the hematological cancer is leukemia or lymphoma.
109. The antigen binding polypeptide complex, antibody or antigen binding fragment thereof or the pharmaceutical composition for use according to clause 108, wherein the leukemia or lymphoma is B cell leukemia, B cell lymphoma, diffuse large B-cell lymphoma (DLBCL) Follicular lymphoma, Chronic lymphocytic leukemia (CLL)/small lymphocytic lymphoma (SLL), Mantle cell lymphoma (MCL), Burkitt lymphoma, Lymphoplasmacytic lymphoma (Waldenstrom macroglobulinemia), prolymphocytic leukemia (PLL) or hairy cell leukemia (HCL).

### EXAMPLES

The following examples are provided to further illustrate aspects of the disclosure, and are not meant to constrain the disclosure to any particular application or theory of operation. SEQ ID NOs:1-866 were obtained and/or prepared according to the processes described herein.

### Example 1

### Design of Masked Multispecific Molecules

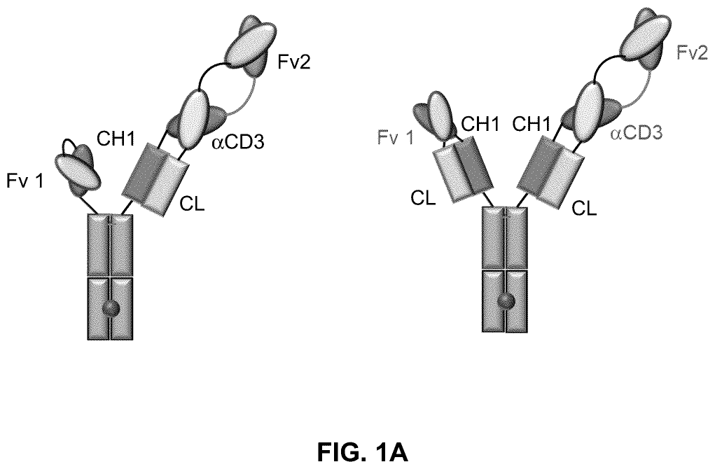

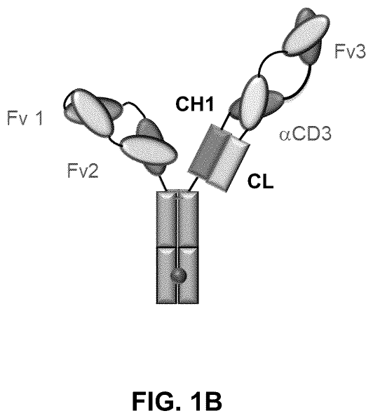

Non-limiting examples of trispecific antibody configurations are shown in FIGS. 1A-1B. FIG. 1A illustrates configurations of masked trispecific T cell engagers. Fv1-Fv2 are against tumor associated antigens (TAAs) or immune costimulatory receptors. The third Fv targets CD3. In some embodiments, linkers between the heavy or light chains of Fv2 and αCD3 contain one or more protease recognition sites. In some embodiments, Fv1-Fv2 target human Trop2, cMet, and/or CD28. In some embodiments, Fv1 is in the form of a scFv. In some embodiments, Fv1 is in the form of a Fab. FIG. 1B illustrates a configuration of masked tetraspecific T cell engagers. Fv1-Fv3 are against tumor associated antigens (TAAs) or immune costimulatory receptors. The fourth Fv targets CD3. In some embodiments, linkers between Fv3 and αCD3 contain one or more protease recognition sites. In some embodiments, Fv1-Fv3 target human Trop2, cMet, and CD28.

Antibody heavy chain variable domain (VH) and light chain variable domain (VL) sequences targeting human CD3, CD28, Trop2, and cMet were selected from publicly available databases (e.g., GenBank) or patents to illustrate the feasibility of constructing various formats of trispecific antibodies. Linkers in various length and sequence connecting VH and VL regions in different orders and orientations were tested, with and without different motifs of the constant domains (e.g., CL, CH1, CH2, CH3). “Knob” and “hole” mutations were integrated into respective halves of the antibody Fc region when Fc heterodimerization was needed. Effector function or half-life extension mutations can also be incorporated into the Fc sequences when needed. Once the amino acid sequences for each trispecific antibody molecule were assembled, DNA encoding these sequences were codon optimized, synthesized (Cambridge Biologics, LLC, Brookline, MA), and cloned into a eukaryotic expression vector.

### Example 2

### Expression and Purification of Masked and Non-Masked Multispecific Molecules

Masked and non-masked antibodies were produced by transient transfection of expression plasmids into Expi293F cells at density of 2.5-3.0×10⁶ per ml using PEI (Polyscience). Plasmid DNA and PEI were diluted in OPTi-MEM (LifeTech) separately and mixed later. The plasmid/PEI mixture, at a ratio of 1:3 (w:w), was added to the cell culture 10 minutes after mixing. Valproic acid and sodium propionate were added to final concentrations of 0.5 mM and 5 mM, respectively, 16-20 hours post transfection. Supernatant was harvested 5 days post transfection, and filtered through a 0.45 μm filter. Multispecific antibodies were then purified first by affinity chromatography using Protein A resins in batch mode according to manufacture's standard procedures. After antibodies were eluted using IgG elusion buffer (Thermo Fischer Scientific) from protein A, they were dialyzed into 10 mM Histidine (pH6.0)+25 mM NaCl overnight. Antibodies were further purified by size exclusion chromatography using Hiload 16/600 Superdex 200 PG or Superdex 200 Increase 10/300 GL (Cytiva Lifesciences). Fractions with the correct elusion profile were collected and concentrated for further characterization.

### Example 3

### In Vitro Protease Treatment of Masked Multispecific Molecules

Purified masked multispecific molecules at 1 ug/ml were incubated with 0.2 pg/ml activated Matriptase (MTP) (R & D systems, Cat #3946-SEB) or 0.4 μg/ml MMP9 (R & D system, Cat #911_MP) at 37° C. for 4 hours. 2 pg of digested proteins were run on SDS-PAGE.

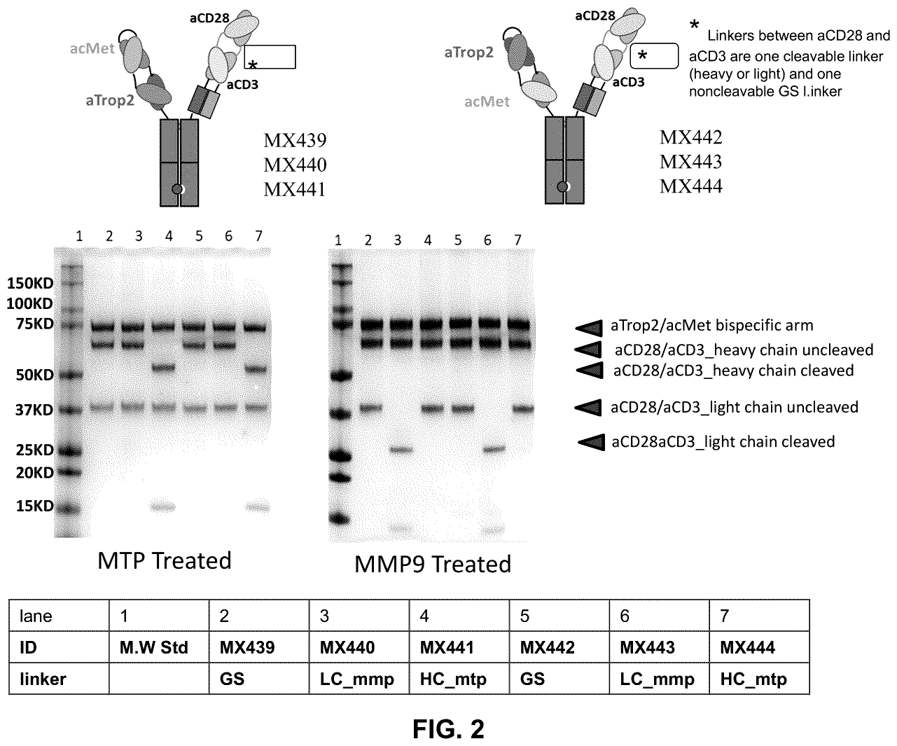

FIG. 2 shows SDS-PAGE results of in vitro cleavage of exemplary masked tetraspecific molecules as depicted. Molecules were treated with either MTP or MMP9 protease as specified. GS:non-cleavable linker sequences are on both light chain (LC) and heavy chain (HC). LC_mmp:MMP2 sensitive linker sequences are on LC, and non-cleavable linker sequences are on HC. HC_mtp:MTP sensitive linker sequences are on HC, and non-cleavable linker sequences are on LC.

### Example 4

### ELISA Binding Analysis of Masked and Non-Masked Multispecific Molecules

An ELISA binding assay was used to test binding of multispecific molecules to their target antigens. Target protein for each binding site of the multispecific molecules was coated in the wells of 96-well Immuno Plate (Thermo Fisher Scientific) overnight at 4° C. Coated plates were blocked using 5% skim milk+2% BSA in PBS+0.25% Tween for one hour at room temperature, then washed with PBS+0.25% Tween 20 for three times. Serial diluted multispecific molecules and control molecules were added to the plate and incubated at room temperature for 1 hour. Plates were washed three times with PBS+0.25% Tween 20, incubated with HPR conjugated detection antibody for one hour at room temperature, washed again, and then developed with Peroxidase Substrate (KPL, Gaithersburg, MD, USA). After the reaction was terminated by adding 100 μl of KPL TMB BlueSTOP solution, the plate was read at OD650 using a plate reader and data analyzed in GraphPad Prism.

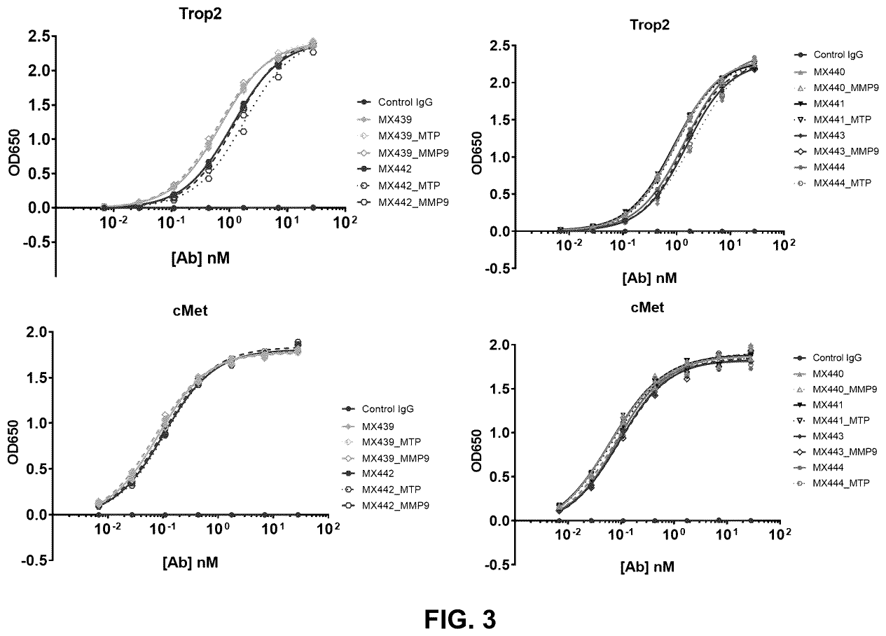

FIG. 3 shows ELISA binding results of exemplary masked tetraspecific molecules as depicted in FIG. 2, or negative isotype (Control IgG1), with or without protease treatment. Molecules cleaved or not cleaved by MTP or MMP9 as specified were tested for binding affinity to Trop2 and cMet. Affinities to these two targets were not affected by protease treatment.

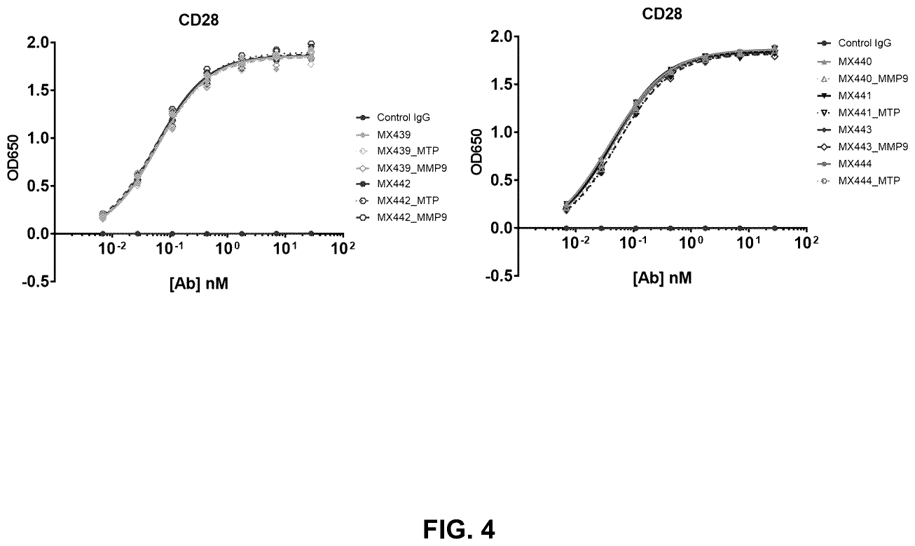

FIG. 4 shows ELISA binding results of exemplary masked tetraspecific molecules as depicted in FIG. 2, or negative isotype (Control IgG1), with or without protease treatment. Molecules cleaved or not cleaved by MTP or MMP9 as specified were tested for binding affinity to CD28. Affinities to these two targets were not affected by protease treatment.

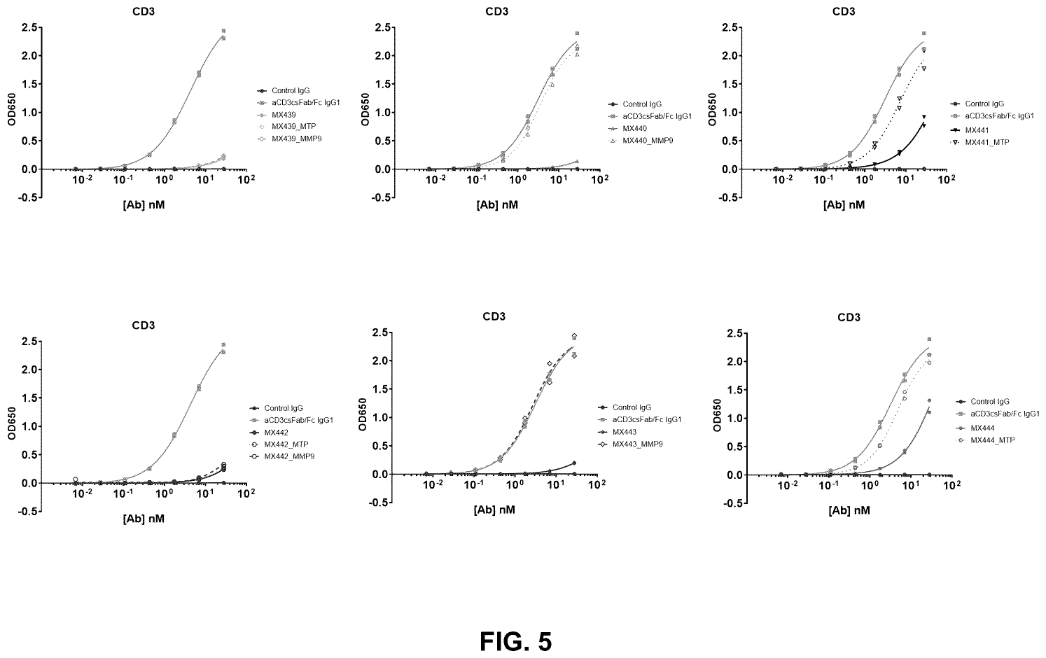

FIG. 5 shows ELISA binding results of exemplary masked tetraspecific molecules as depicted in FIG. 2, or negative isotype (Control IgG1), with or without protease treatment. Molecules cleaved or not cleaved by MTP or MMP9 as specified were tested for binding affinity to CD3.

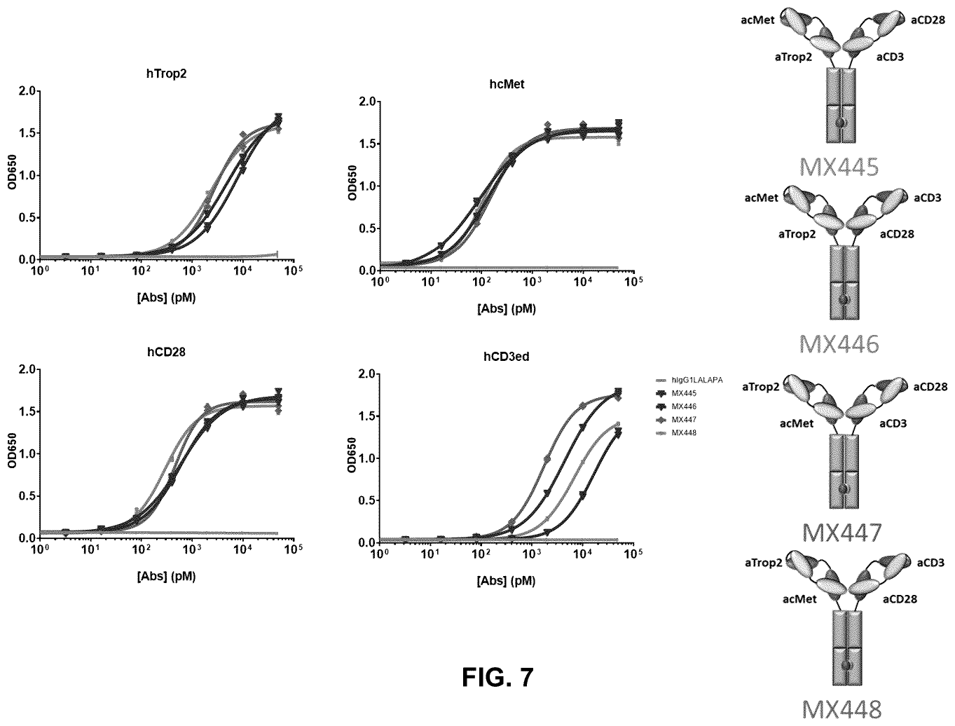

FIG. 7 shows ELISA binding results of exemplary non-masked tetraspecific molecules as depicted, or negative isotype (hIgG1LALPA) control, to their respective targets of hTrop2, hcMet, hCD28, and hCD3.

### Example 5

### T Cell Activation Assay

T cell activation by multispecific molecules was tested using an in vitro T cell activation assay. Purified human PBMCs (Blood Research Component, Brookline, MA, USA) were resuspended in culture medium (RPMI1640 with 10% FBS and supplemented with Penicillin Streptomycin)(Gibco) (2.5×10⁵ cells/ml). Serial diluted multispecific and control molecules were first coated onto 96-well flat-bottom culture plates by incubating for 2-4 hours in a 37° C. tissue culture incubator. PBMCs (200 μL) were then added to each well containing the molecules and incubated for 16-24 hours in a 37° C. tissue culture incubator. The cells were spun down, stained with florescent labeled antibodies for T cell activation marker CD69, and analyzed by an Attune flow cytometer (Thermo Fisher Scientific, USA). Data was analyzed using FlowJo software.

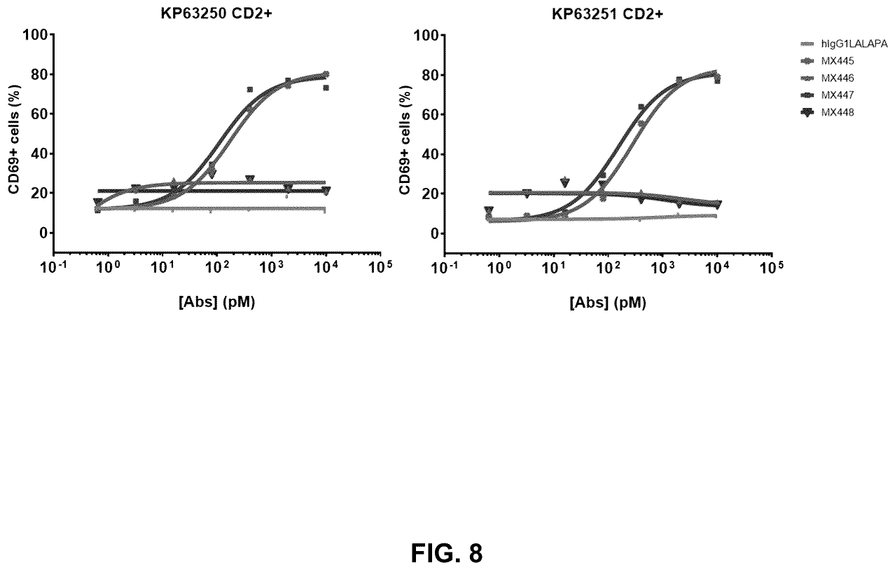

FIG. 8 shows CD69+ activation by exemplary non-masked tetraspecific molecules, or negative isotype (IgG1LALPA) control, of CD2+ T cells from PBMCs of two different donors.

### Example 6

### In Vitro Cytolytic Assay

Cytolysis of lymphoma tumor cells Z-138 by T cells mediated by trispecific antibodies was determined using an in vitro cytolytic assay. PBMCs were isolated from normal human donors by Ficoll separation. In vitro cytotoxicity assay was real-time monitored of cellular phenotypic changes by measurement of electrical impedance using the Agilent×CELLigence RTCA MP system. The system measures impedance using interdigitated microelectrodes integrated into the bottom of each well of the tissue culture E-Plates 96. Briefly, tumor cell HCC1954 were seeded into an E-plate 96 as target cells (T) at 20K/well culture at 37° C. for overnight, followed by the addition of human PBMC cells as immune effector cells (E) at 200K/well, in the presence of the 5-fold serially diluted multispecific antibody or human IgG1 isotype control. Cell impedance (measured as the cell index) was normalized when the effector cells were added and monitored continuously every 30 min for a duration of up to 160 hours. The cytotoxicity was calculated as Lysis %=100−(experimental normalized cell index/average of control antibody group normalized cell index at same concentration)×100.

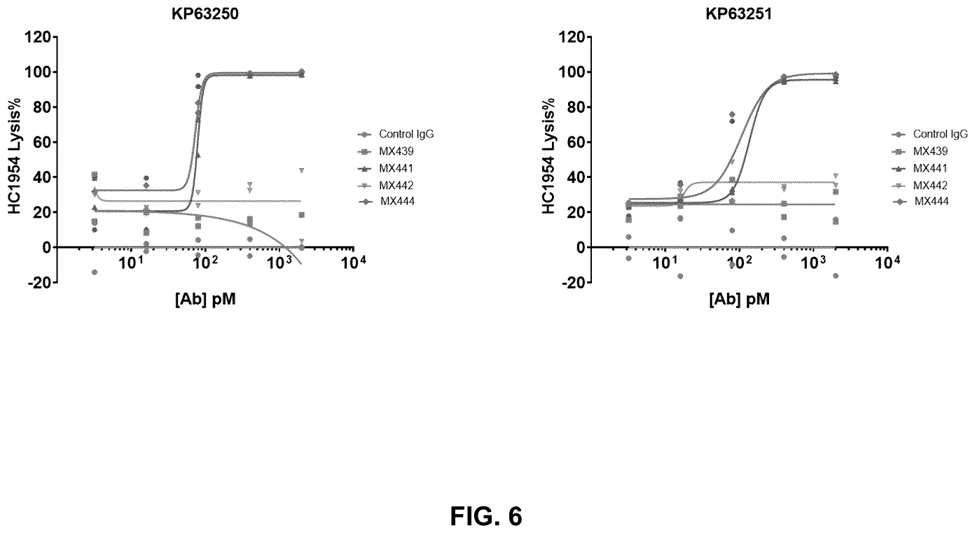

FIG. 6 shows cytolysis of HCC1954 tumor cells by PBMCs (E:T:10:1) mediated by exemplary masked tetraspecific molecules as depicted in FIG. 2, or negative isotype (Control IgG1), from PBMCs of two donors (KP63250 and KP63251).

### Example 7

### ELISA Binding Results of Exemplary Non-Masked Tetraspecific Molecules to their Targets hTrop2, hcMet, hCD28, and hCD3

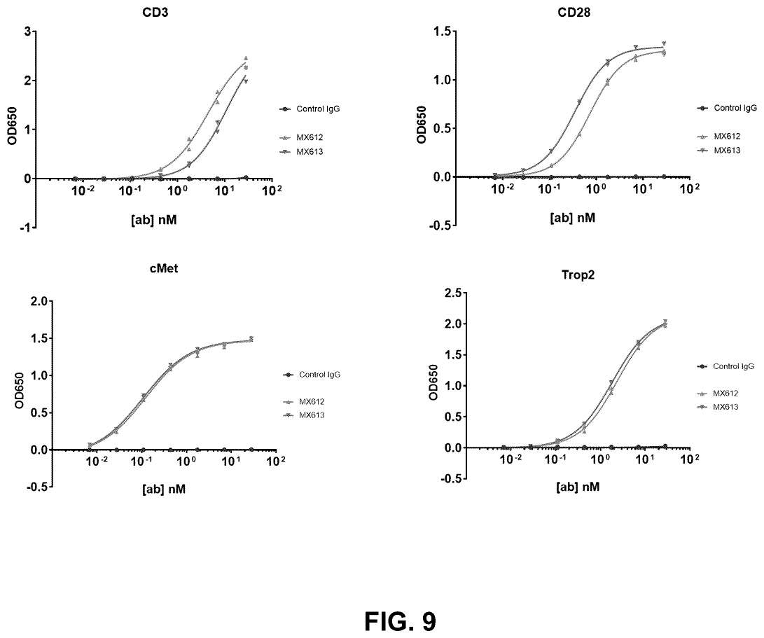

Non-masked tetraspecific molecules MX612 (peptide: SEQ ID NO:250 (DNA: SEQ ID NO: 251), peptide: SEQ ID NO:412 (DNA: SEQ ID NO:413)) and MX613 were prepared as described above with binding regions for human cMet (hcMet), human Trop2 (hTrop2), human CD3 (hCD3), and human CD28 (hCD28). Binding affinity was confirmed by ELISA. FIG. 9 shows ELISA binding results (OD650) of MX612 and MX613 to their targets hTrop2, hcMet, hCD28, and hCD3. Results from a control antibody (control IgG), which does not bind these targets, is also shown. These results show that MX612 and MX613 bind to hTrop2, hcMet, hCD28, and hCD3.

### Example 8

### Simultaneous Co-Binding of MX612 to its Targets hTrop2, hcMet, hCD28, and hCD3 Measured by Biolayer-Interferometry

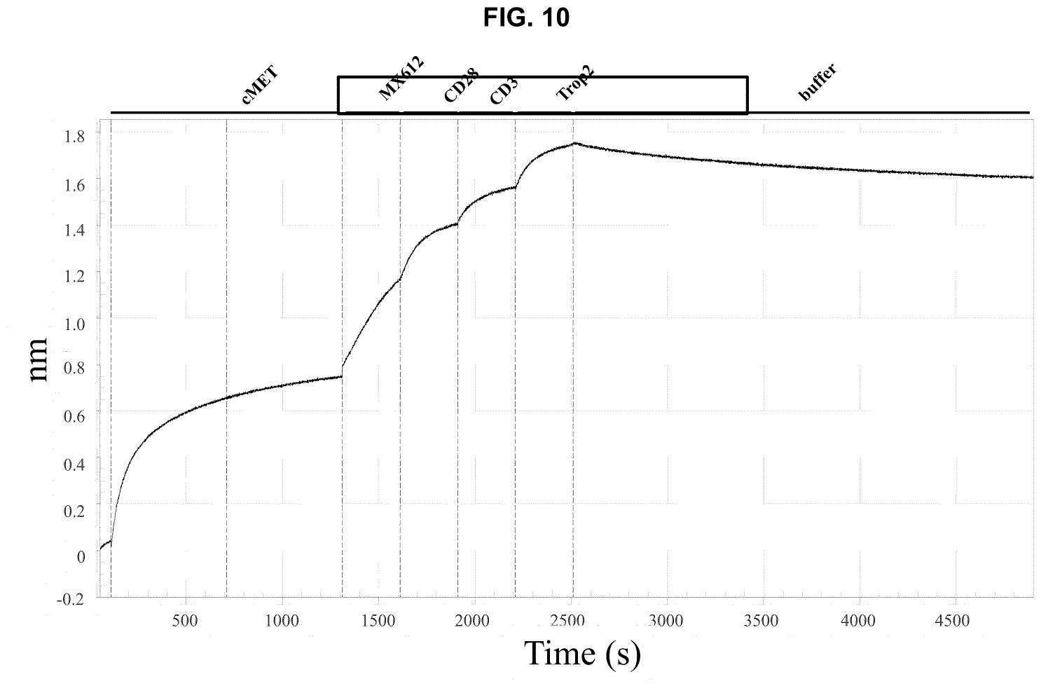

Simultaneous co-binding of MX612 to its targets hTrop2, hcMet, hCD28, and hCD3 was measured by biolayer-interferometry. On the Octet R8 (Sartorius), each equilibrated HIS1K (his-tag capture) biosensor was dipped into buffer for 60 seconds, followed by loading with first ligand to near-saturation (1400 or 1200 seconds as indicated). Afterwards, sequential analysis steps were performed with analyte (ligand or multispecific test antibody) or buffer as indicated for time frame as indicated. Plate oscillation at 1000 RPM; 1× kinetic buffer (1×PBS pH 7.4; 0.1% BSA; 0.02% Tween-80); 24 degrees. For biolayer-interferometry, His-tagged recombinant Trop2, cMET, CD28, or CD3 (100 nM in solution) was loaded onto a solid support biosensor by His-tag capture. MX612 was present as a solution analyte (52 nM). Association and disassociation were evaluated for 300 seconds each. The binding reaction occurred in 1× kinetic buffer (1×PBS pH 7.4; 0.1% BSA; 0.2% Tween-80) at 24 degrees. Results are shown in FIG. 10.

### Example 9

### Simultaneous Co-Binding of MX446 to its Targets hTrop2, hcMet, hCD28, and hCD3 Measured by Biolayer-Interferometry

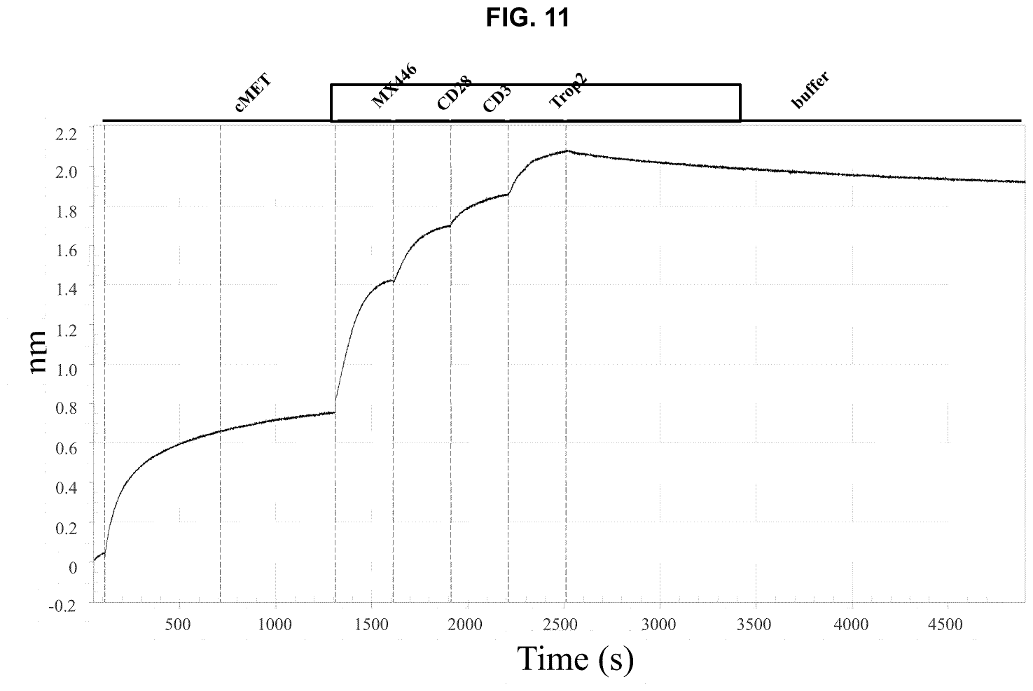

Simultaneous co-binding of MX446 (peptide: SEQ ID NO:206 (DNA: SEQ ID NO:207), peptide: SEQ ID NO:511 (DNA: SEQ ID NO:512)) to its targets hTrop2, hcMet, hCD28, and hCD3 was measured by biolayer-interferometry as explained above. Results are shown in FIG. 11.

### Example 10

### CD4+ and CD8+ T Cell Activation by Exemplary Non-Masked Tetraspecific Molecules

The ability of exemplary non-masked tetraspecific molecules to activate CD4+ and CD8+ T cells (CD69+) was tested. Human PBMCs (Blood Research Component, Brookline, MA, USA) were purified from healthy donors, resuspended in culture medium (RPMI1640 with 10% FBS and supplemented with Penicillin/Streptomycin)(Gibco), and seeded onto 96-well cell culture plates containing serially diluted multispecific test antibodies or human IgG1LALAPA isotype control. The plates were incubated for 16-24 hours in a 37° C. tissue culture incubator. The cells were then stained with fluorescently labeled antibodies (anti-CD2, anti-CD8, anti-CD4, anti-CD69, and fixable viability dye) and analyzed by flow cytometry (Thermo Fisher Scientific, USA) for measuring percentage of T cell activation marker CD69 among T cell subsets. Data were analyzed using FlowJo software.

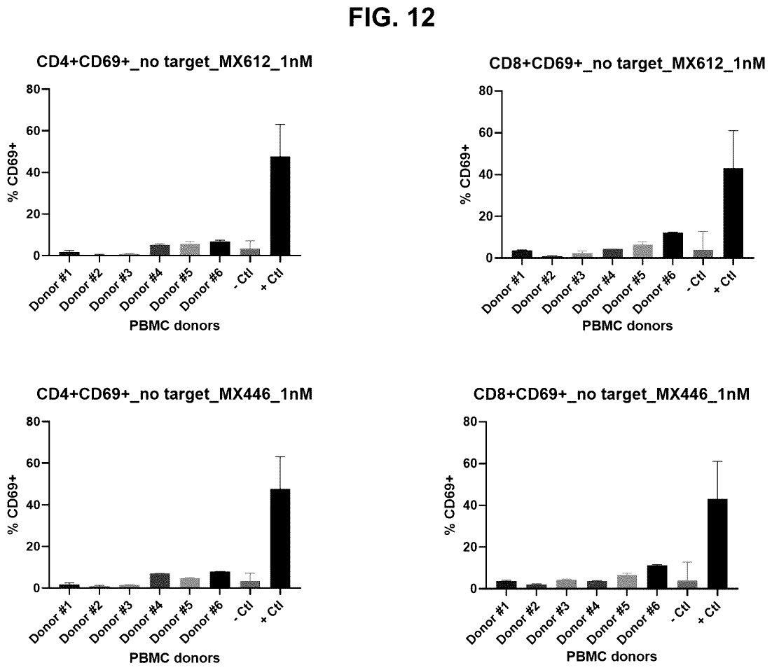

Results from PBMCs from six different donors in the absence of target tumor cells and following treatment with 1 nM MX446 and MX612 are shown in FIG. 12. Results from a negative control (isotype IgG1LALPA; “−Ctl”) and positive control (CD3+CD28 mAbs; “+Ctl”) are also shown.

### Example 11

### In Vitro Cytokine Release by Exemplary Non-Masked Tetraspecific Molecules

The ability of exemplary non-masked tetraspecific molecules to initiate release of the cytokines IL-2, IFN-gamma, IL-6 and TNFalpha was tested. Cell culture supernatants were assayed for cytokines using the MILLIPLEX® MAP multiplex assay (Millipore Sigma) with adoption of the Drop Array system (Curiox Biosystems, Singapore). In brief, cell culture supernatants were collected 24 or 48 hours after co-incubation of human PBMC and test antibodies along with, or without, tumor target cells. Magnetic analyte bead mixture was then added to wells in the DropArray assay plate. Standards, quality controls, and diluted samples were then added to the plate; all standards and quality controls were tested in duplicate, with samples tested in multiple replicates. The plate was placed on a magnetic stand in a humidified chamber and shaken overnight at 4° C. The plate was washed with a DropArray LT210 washing station (Curiox Biosystems). Detection antibody and streptavidin-PE substrate were added to each well and incubated with shaking. The plate was washed before reading by Luminex MAGPIX instrument. Data were analyzed using EMD Millipore Milliplex Analyst software or Thermofisher Procartaplex Analysis Tool.

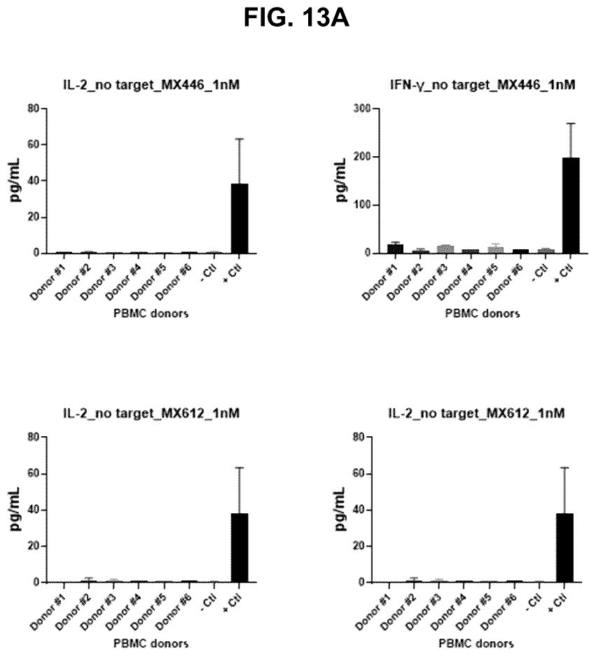

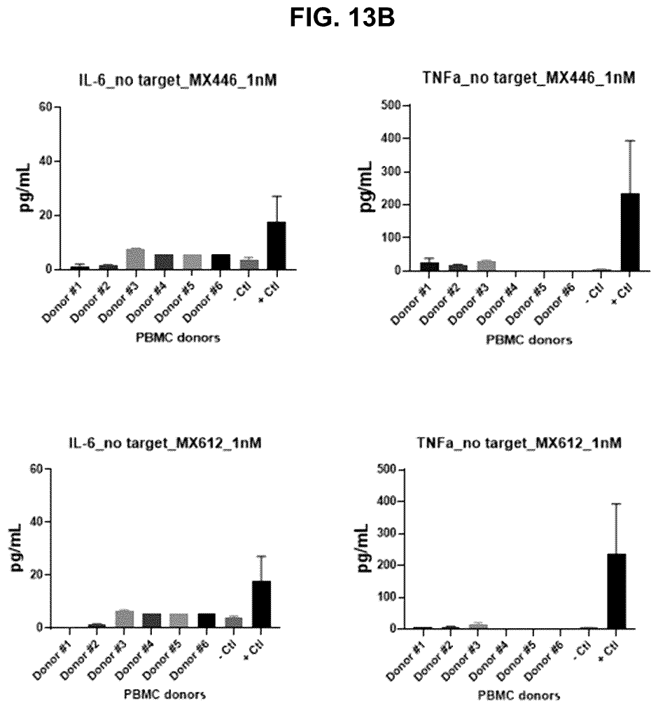

Results from PBMCs from six different donors in the absence of target tumor cells and following treatment with 1 nM MX446 and MX612 are shown in FIG. 13A-13B. Results from a negative control (isotype IgG1LALPA; “−Ctl”) and positive control (CD3+CD28 mAbs; “+Ctl”) are also shown.

### Example 12

### In Vitro Cytokine Release by Exemplary Non-Masked Tetraspecific Molecules

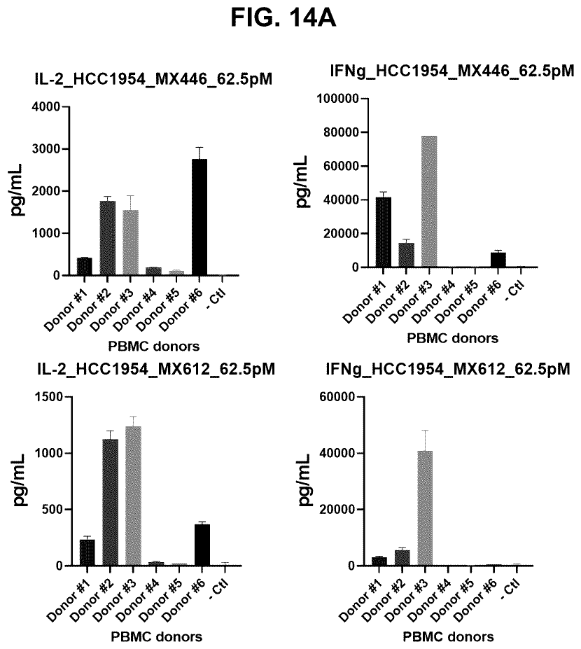

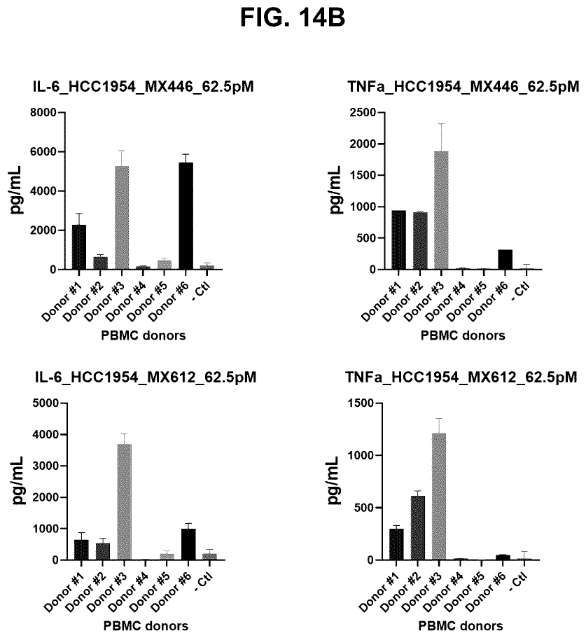

The ability of exemplary non-masked tetraspecific molecules to initiate release of the cytokines IL-2, IFN-gamma, IL-6 and TNFalpha was tested. Results from PBMCs from six different donors in the presence of HCC1954 tumor cells and following treatment with 62.5 pM MX446 and MX612 are shown in FIG. 14A-14B. Results from a negative control (isotype IgG1LALPA; “−Ctl”) are also shown.

### Example 13

### In Vitro Cytokine Release by Exemplary Non-Masked Tetraspecific Molecules

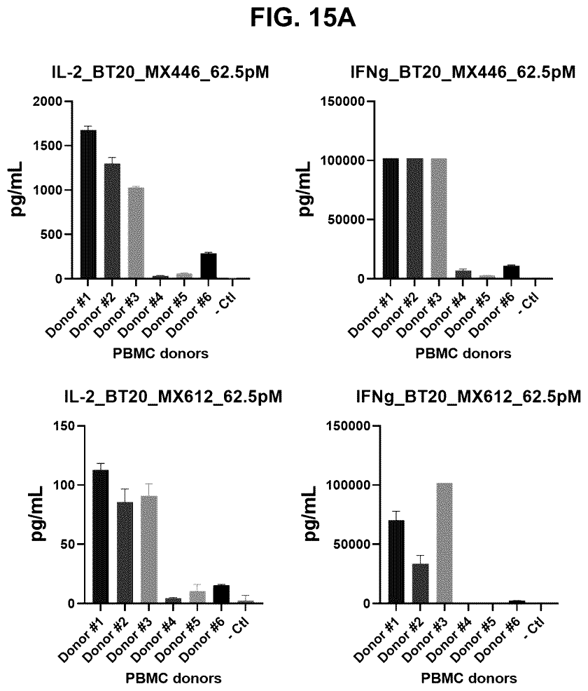

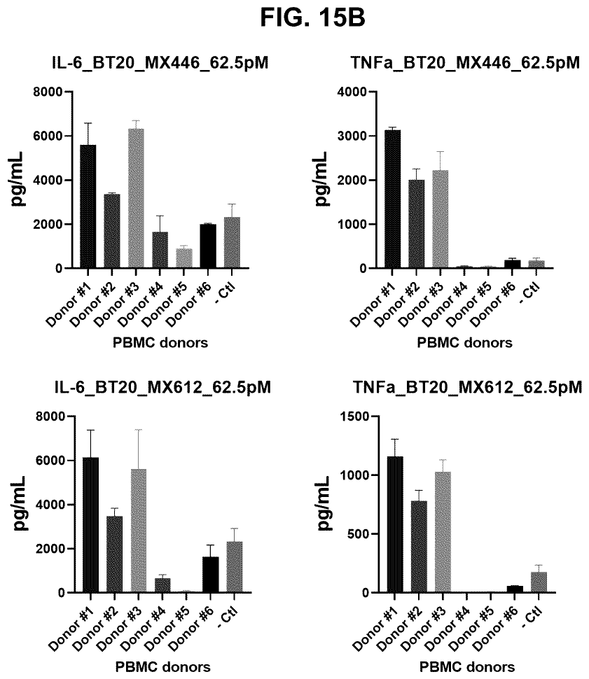

The ability of exemplary non-masked tetraspecific molecules to initiate release of the cytokines IL-2, IFN-gamma, IL-6 and TNFalpha was tested. Results from PBMCs from six different donors in the presence of BT20 tumor cells and following treatment with 62.5 pM MX446 and MX612 are shown in FIG. 15A-15B. Results from a negative control (isotype IgG1LALPA; “−Ctl”) are also shown.

### Example 14

### Cytolysis of Tumor Cell Lines Mediated by Exemplary Non-Masked Tetraspecific Molecules

Cytolysis of tumor cell lines by exemplary non-masked tetraspecific molecules or a negative control from PBMCs (E:T 5:1) was tested. The target tumor cells were labeled with the membrane dye PKH-26 (Sigma-Aldrich) and seeded into 96-well U bottom plate at 20K/well. The serially diluted test multispecific antibodies or human IgG1LALAPA isotype control were added with human PBMCs or enriched Pan-T cells as effector cells at an effector-to-target (E:T) ratio of 5:1 (E:T=3:1 using enriched Pan T cells). After culturing at 37° C. for 24 hours, the cells were staining with a LIVE/DEAD Fixable Dead Cell Stain Kit (Life Technologies) and measured by the number of live cells in the labeled target cell population by running the samples on Attune instrument (ThermoFisher) followed by analysis using the FlowJo software (Tree Star). The cytotoxicity was calculated as Lysis %=100−(experimental live target cell/average of control antibody group live target cell)*100.

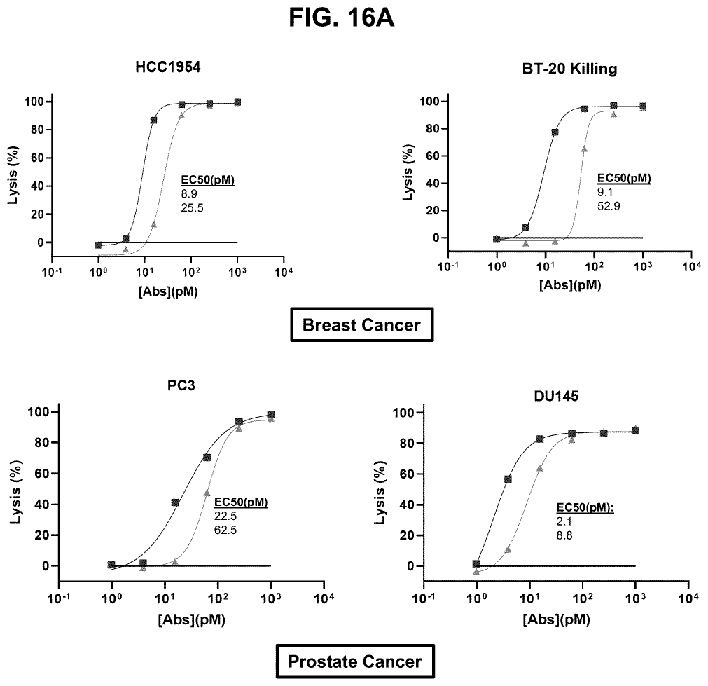

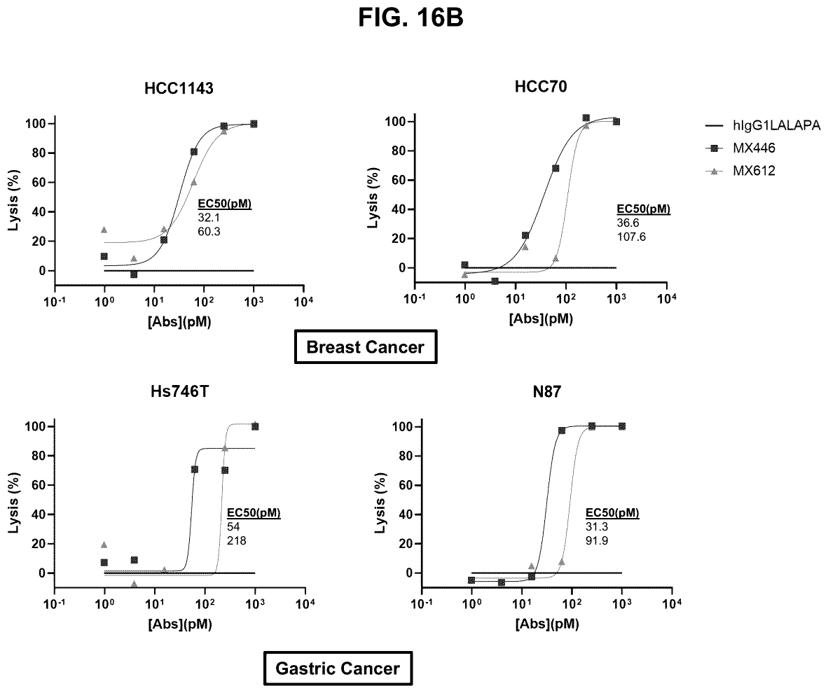

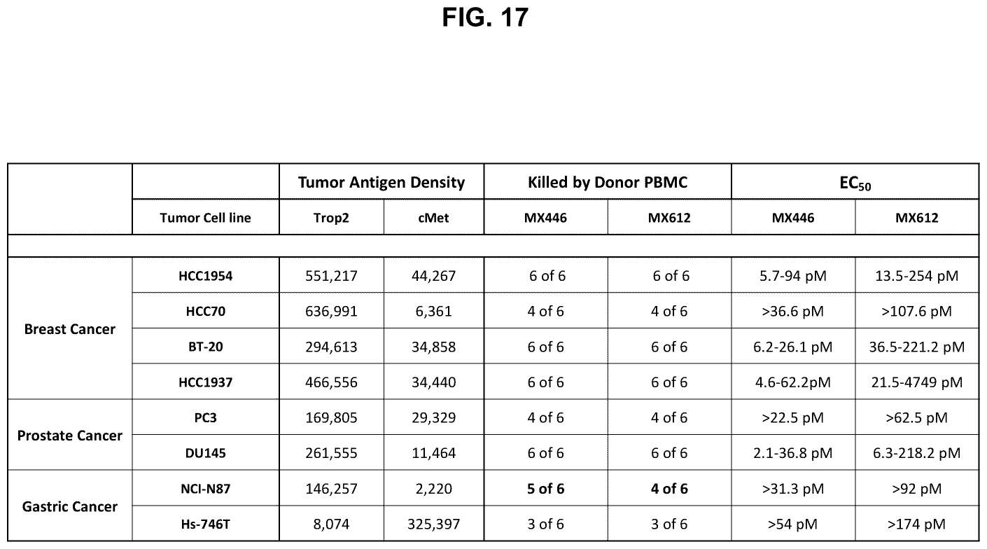

HCC1954, BT-20, HCC1143 and HCC70 breast cancer cells, PC3 and DU145 prostate cancer cells, and Hs746T and N87 gastric cancer cells were used. Percent lysis of cells following treatment with MX446 and MX612, and EC50 values in pM are shown in FIG. 16A-16B. Results from a negative control (isotype IgG1LALAPA) are also shown. FIG. 17 summarizes cytolysis potencies of various tumor cell lines mediated by exemplary non-masked tetraspecific molecules, from PBMCs (E:T: 5:1) of six different donors.

### Example 15

### Binding of Exemplary Non-Masked Tetraspecific Molecules to hTrop2, hcMet, hCD28, and hCD3

Binding of exemplary non-masked tetraspecific molecules to their targets hTrop2, hcMet, hCD28, and hCD3 was measured by biolayer-interferometry. On the Octet R8 (Sartorius), recombinant his-tagged Trop2, cMet, CD3, CD28 or CD19 as indicated was loaded by his-tag capture onto HIS1K biosensors (100-300 nM ligand, 300 seconds, 1000 RPM). After baseline step (100 seconds, 1000 RPM), loaded onto HIS1K biosensors (Sartorius) (300 seconds). Following baseline step (100 seconds), association with analyte multispecific test antibody as indicated (52 nM; 300 seconds) and dissociation (300 seconds) was monitored. All steps in 1× kinetic buffer (1×PBS pH 7.4; 0.1% BSA; 0.02% Tween-80), at 24 degrees, and with 1000 RPM. Baseline was set by mean of last five seconds of baseline step and high-frequency noise was filtered by Savitsky-Golay filtering (Octet Analysis Studio, Sartorius).

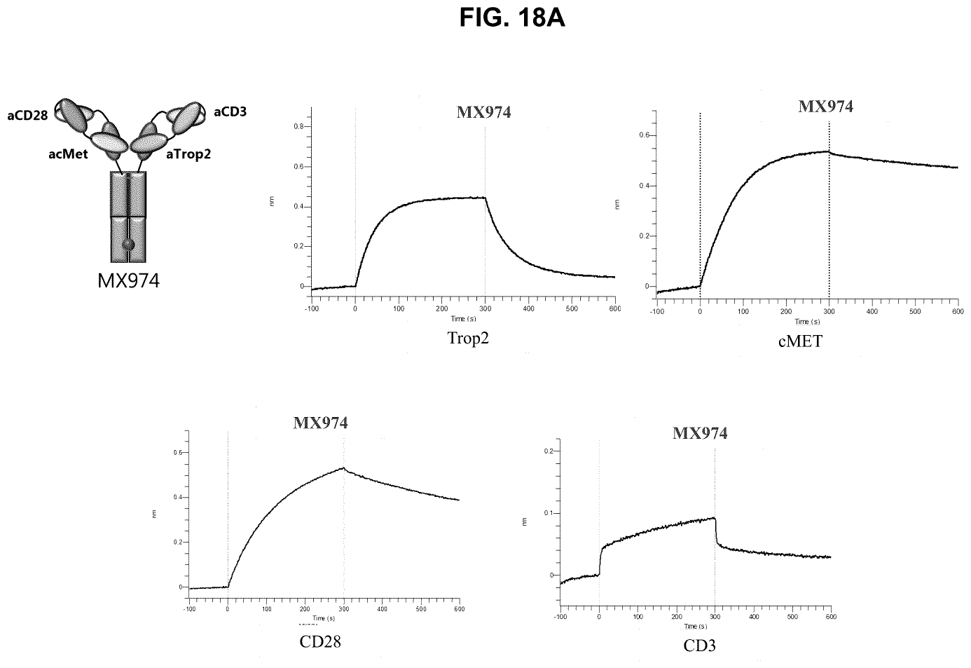

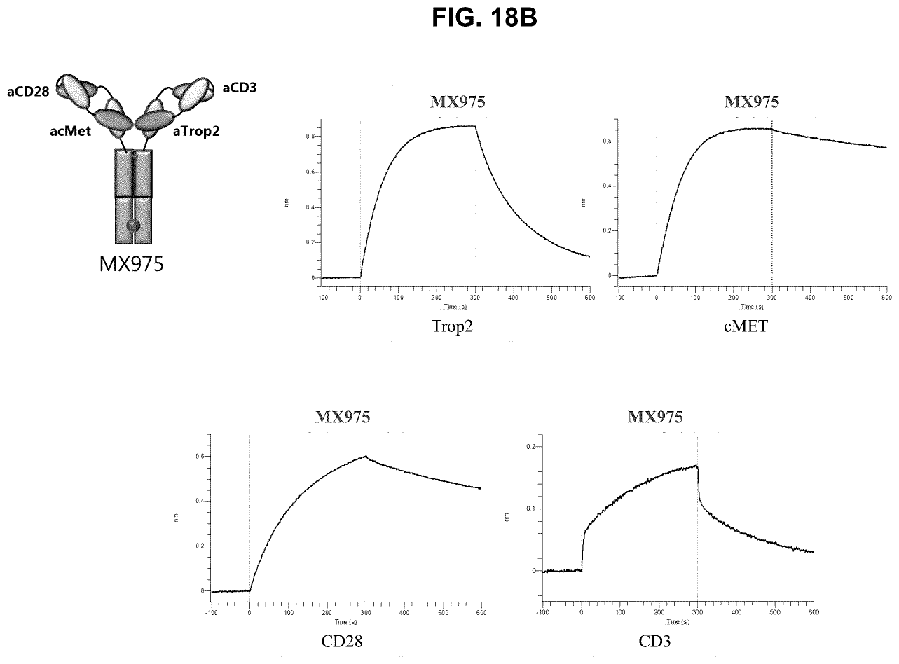

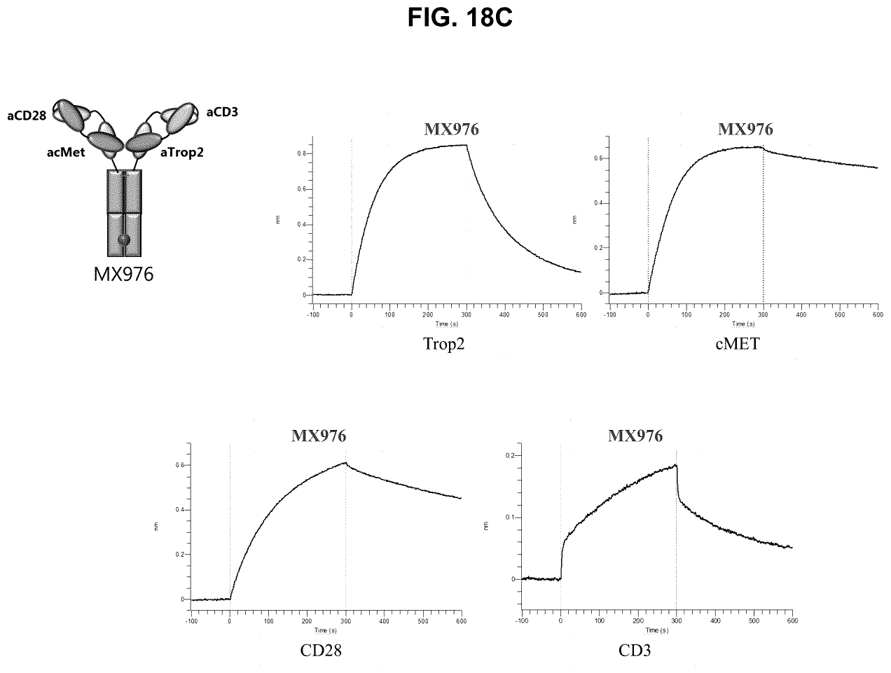

Results are shown in FIG. 18A-18C for cells treated with MX974, MX975 and MX976, respectively. These results show that MX974, MX975 and MX976 bound to hTrop2, hcMet, hCD28, and hCD3.

### Example 16

### In Vitro Cytolysis of HCC1954 Tumor Cells Mediated by Exemplary Non-Masked Tetraspecific Molecules

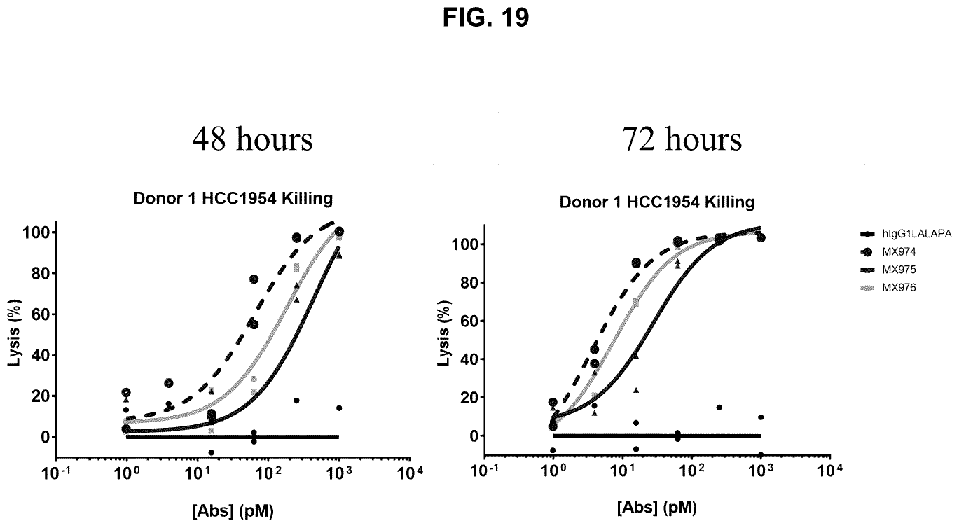

In vitro cytolysis of HCC1954 tumor cells by exemplary non-masked tetraspecific molecules was measured. Cytolysis mediated by MX974, MX975 and MX976 or a negative control from PBMCs (E:T: 5:1) was measured from a representative donor after 48 and 72 hours of incubation at 37° C. Results are shown in FIG. 19 as percent lysis of cells with increasing concentration of antibody. Results from a negative control (hIgG1LALAPA) are also shown.

### Example 17

### CD4+ and CD8+ T Cell Activation (CD69+) by Exemplary Masked Tetraspecific Molecules

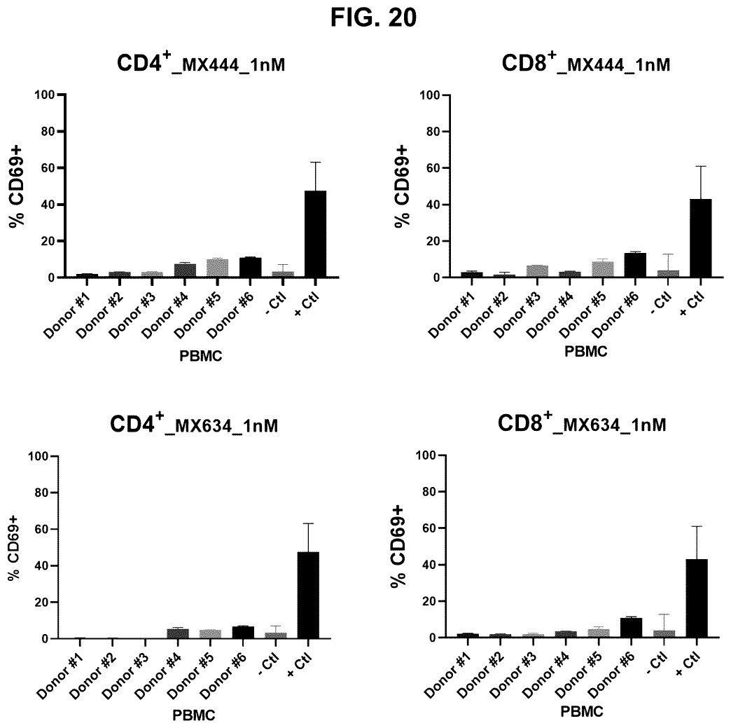

Activation of CD4+ and CD8+ T cells (CD69+) from PBMCs by exemplary masked tetraspecific molecules was tested. Results are shown in FIG. 20 as the percentage of CD69+ cells from six donors in the absence of target tumor cells, following treatment with MX444 (peptide: SEQ ID NO:204 (DNA: SEQ ID NO:205), peptide: SEQ ID NO:162 (DNA: SEQ ID NO: 163), peptide: SEQ ID NO:176 (DNA: SEQ ID NO:177)) or MX634 (peptide: SEQ ID NO:372 (DNA: SEQ ID NO:373), peptide: SEQ ID NO:426 (DNA: SEQ ID NO:427), peptide: SEQ ID NO:469 (DNA: SEQ ID NO:470)) at 1 nM. Results from a negative control (isotype IgG1LALPA; “−Ctl”) and a positive control (CD3+CD28 mAbs; “+Ctl”) are also shown.

### Example 18

### In Vitro Cytokine Release by Exemplary Masked Tetraspecific Molecules

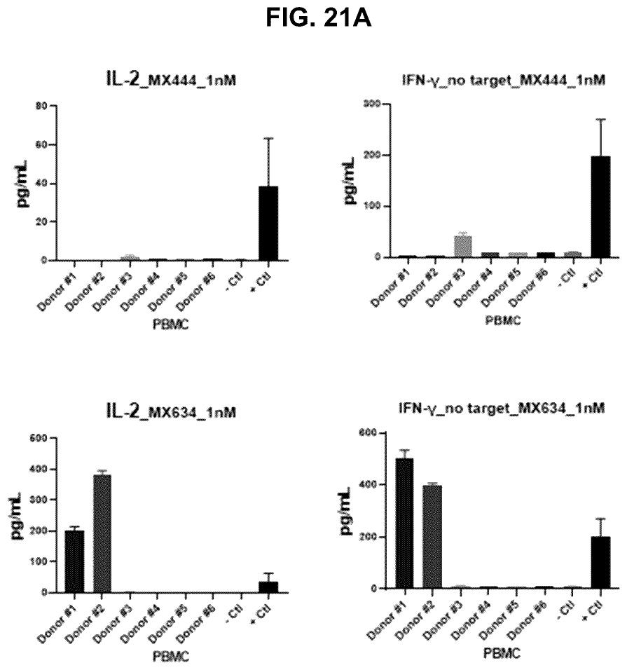

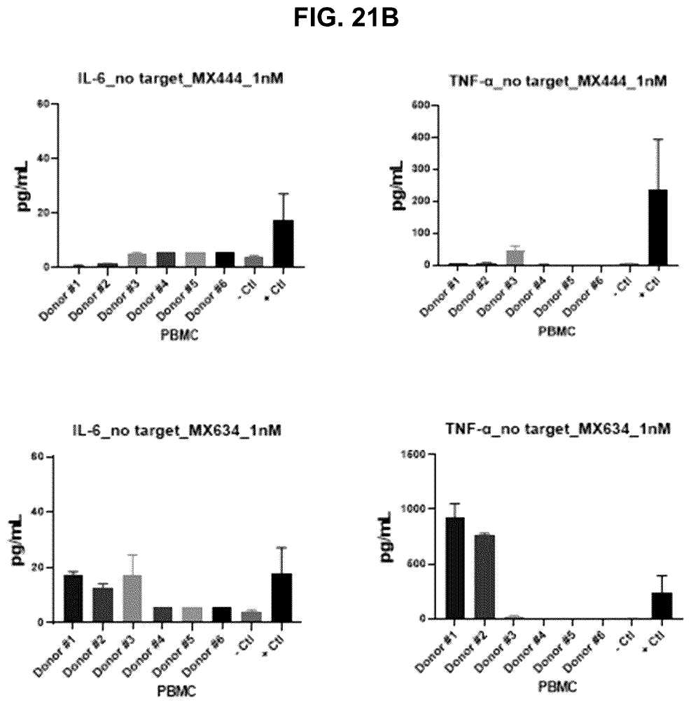

The ability of exemplary non-masked tetraspecific molecules to initiate release of the cytokines IL-2, IFN-gamma, IL-6 and TNFalpha was tested. Results from PBMCs from six different donors in the absence of target tumor cells and following treatment with 1 nM MX444 and MX634 are shown in FIG. 21A-21B. Results from a negative control (isotype IgG1LALPA; “−Ctl”) and positive control (CD3+CD28 mAbs; “+Ctl”) are also shown.

### Example 19

### In Vitro Cytokine Release by Exemplary Masked Tetraspecific Molecules

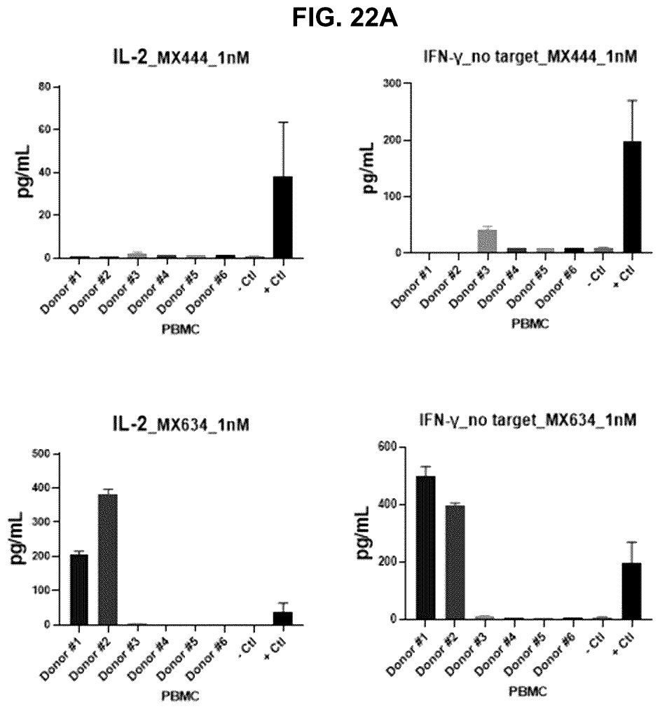

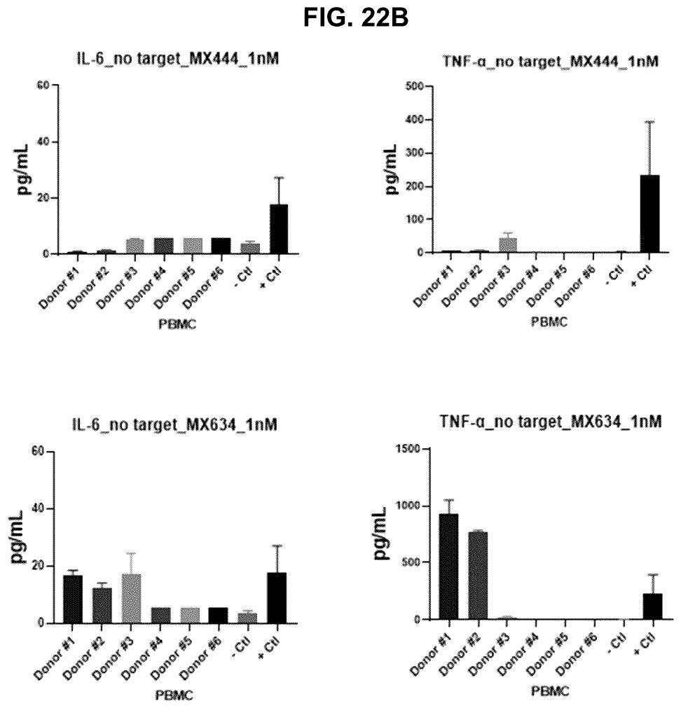

The ability of exemplary non-masked tetraspecific molecules to initiate release of the cytokines IL-2, IFN-gamma, IL-6 and TNFalpha was tested. Results from PBMCs from six different donors in the absence of target tumor cells and following treatment with 1 nM MX444 and MX634 are shown in FIG. 22A-22B. Results from a negative control (isotype IgG1LALPA; “−Ctl”) and positive control (CD3+CD28 mAbs; “+Ctl”) are also shown.

### Example 20

### In Vitro Cytokine Release by Exemplary Masked Tetraspecific Molecules

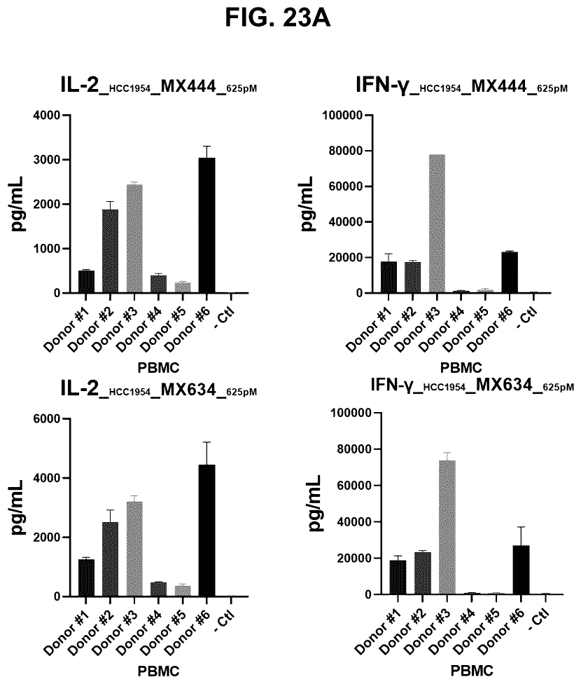

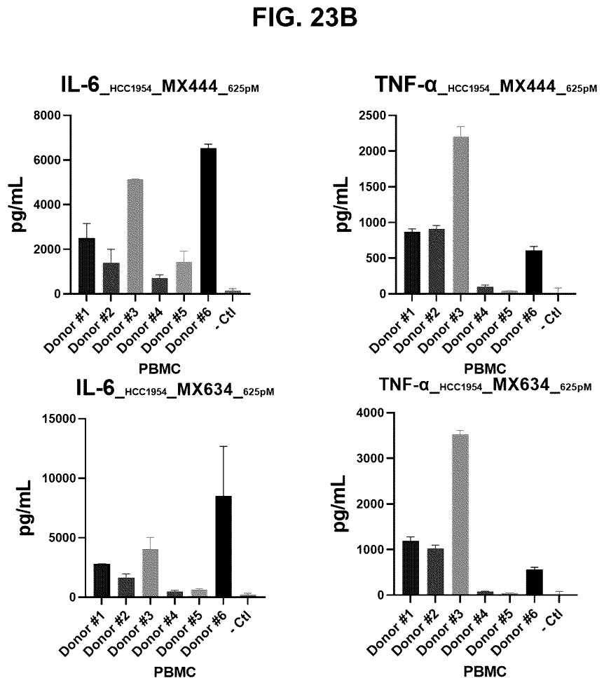

The ability of exemplary non-masked tetraspecific molecules to initiate release of the cytokines IL-2, IFN-gamma, IL-6 and TNFalpha was tested. Results from PBMCs from six different donors in the presence of HCC1954 tumor cells and following treatment with 62.5 pM MX444 and MX634 are shown in FIG. 23A-23B. Results from a negative control (isotype IgG1LALPA; “−Ctl”) are also shown.

### Example 21

### In Vitro Cytokine Release by Exemplary Masked Tetraspecific Molecules

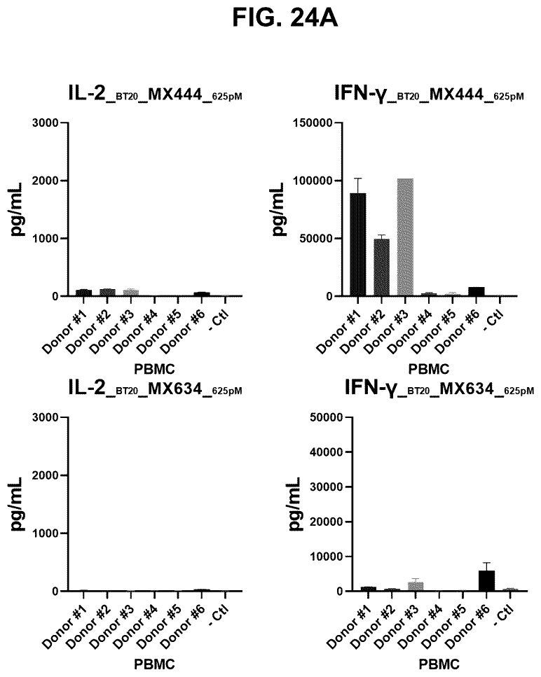

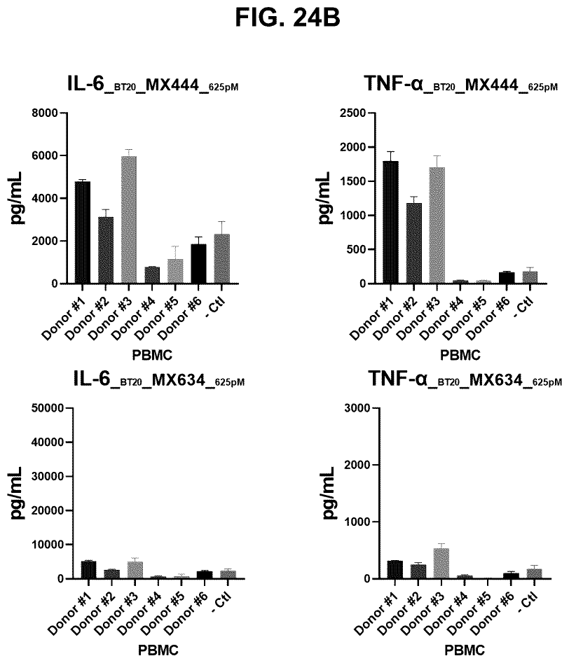

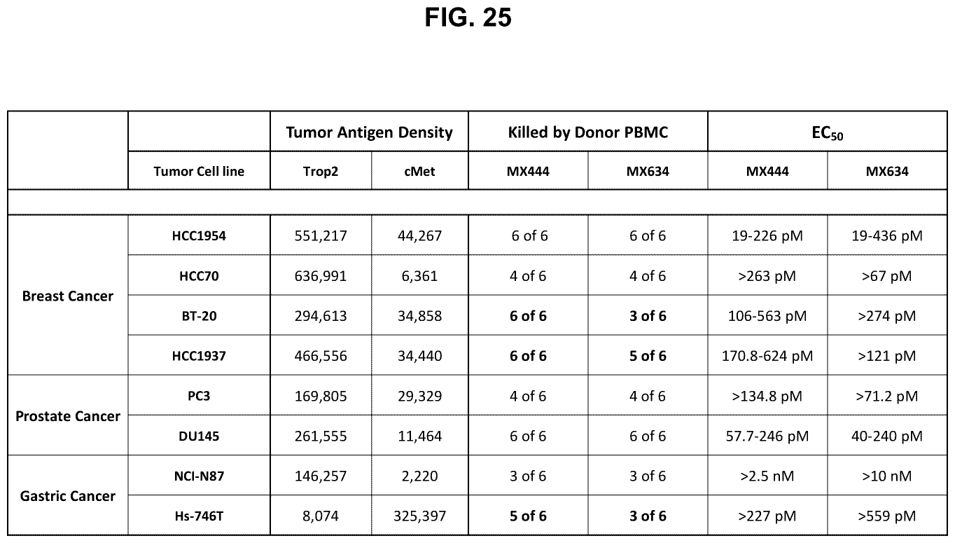

The ability of exemplary non-masked tetraspecific molecules to initiate release of the cytokines IL-2, IFN-gamma, IL-6 and TNFalpha was tested. Results from PBMCs from six different donors in the presence of BT20 tumor cells and following treatment with 62.5 pM MX444 and MX634 are shown in FIG. 24A-24B. Results from a negative control (isotype IgG1LALPA; “−Ctl”) are also shown. FIG. 25 summarizes cytolysis potencies of various tumor cell lines mediated by exemplary non-masked tetraspecific molecules, from PBMCs (E:T: 5:1) of six different donors.

### Example 22

### ELISA Binding Results of Exemplary Non-Masked Tetraspecific Molecules to hCD19, hCD28 and hCD3

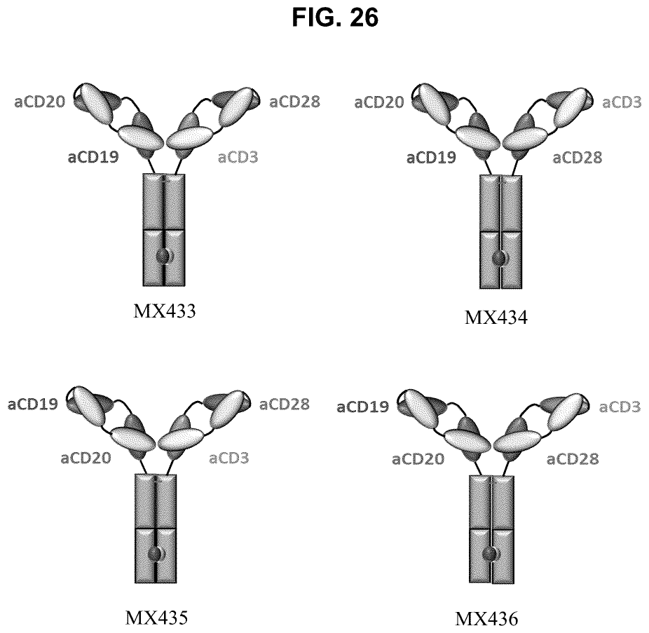

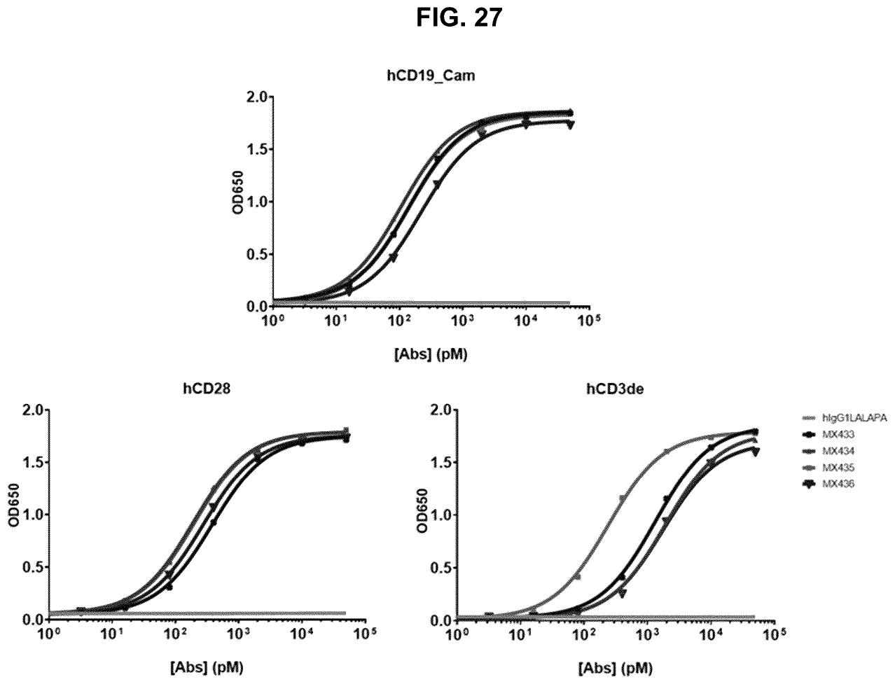

The ability of exemplary non-masked tetraspecific molecules MX433, MX434, MX435 and MX436 to bind to their targets was tested by ELISA. The molecules tested are shown in FIG. 26, and binding results to hCD19, hCD28 and hCD3 are shown in FIG. 27. Results from a negative control (hIgG1LALPA) are also shown.

### Example 23

### ELISA Binding Results of Exemplary Non-Masked Tetraspecific Molecules to hCD19, hCD28 and hCD3

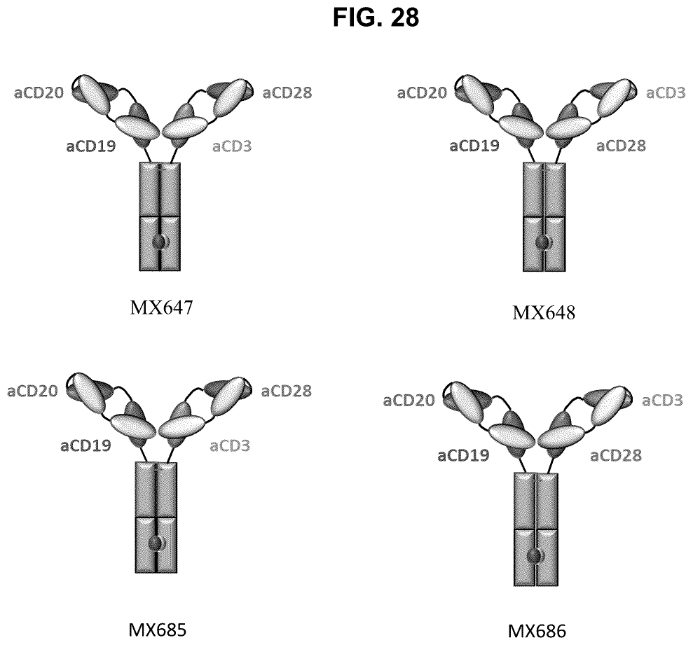

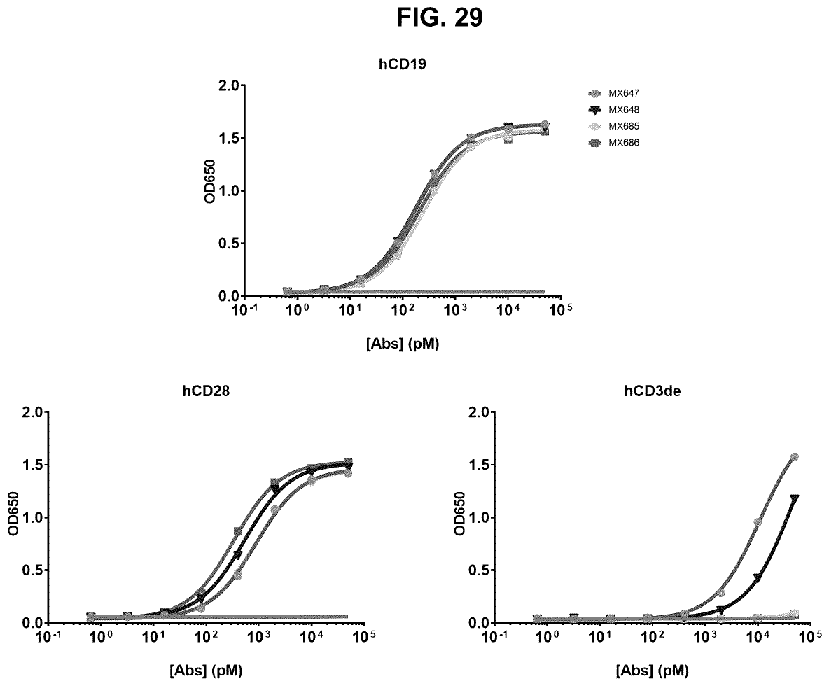

The ability of exemplary non-masked tetraspecific molecules MX647, MX648, MX685 and MX686 to bind to their targets was tested by ELISA. The molecules tested are shown in FIG. 28, and binding results to hCD19, hCD28 and hCD3 are shown in FIG. 29.

### Example 24

### Surface Staining of Exemplary Non-Masked Tetraspecific Molecules

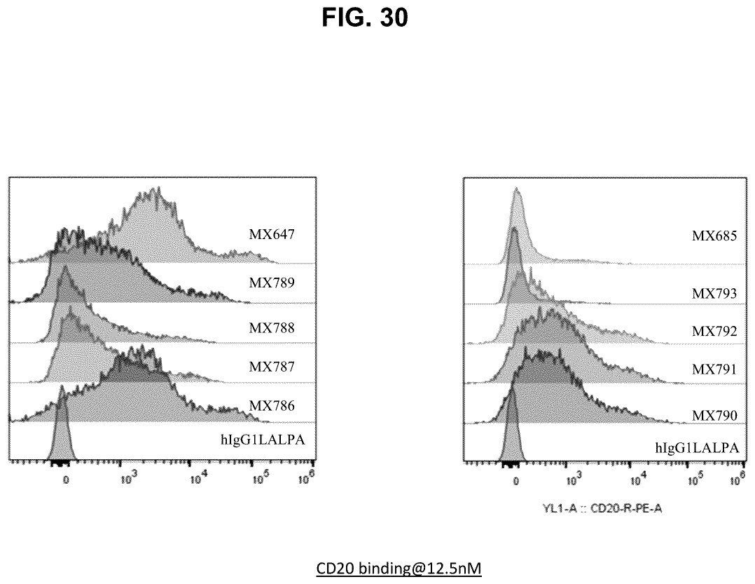

Surface staining of exemplary non-masked tetraspecific molecules to hCD20-expressing Epi293 cells was performed. The results are shown in FIG. 30. The molecules tested were MX647 (peptide: SEQ ID NO:513 (DNA: SEQ ID NO:514), peptide: SEQ ID NO:563 (DNA: SEQ ID NO:564)), MX789, MX788, MX787, MX786, MX685 (peptide: SEQ ID NO:513 (DNA: SEQ ID NO:514), peptide: SEQ ID NO:591 (DNA: SEQ ID NO:592)), MX793, MX792, MX791 and MX790. Staining with a negative control (hIgG1LALPA) is also shown.

### Example 25

### T Cell Activation, In Vitro Cytolysis, and In Vitro Cytokine Release by Exemplary Non-Masked Tetraspecific Molecules

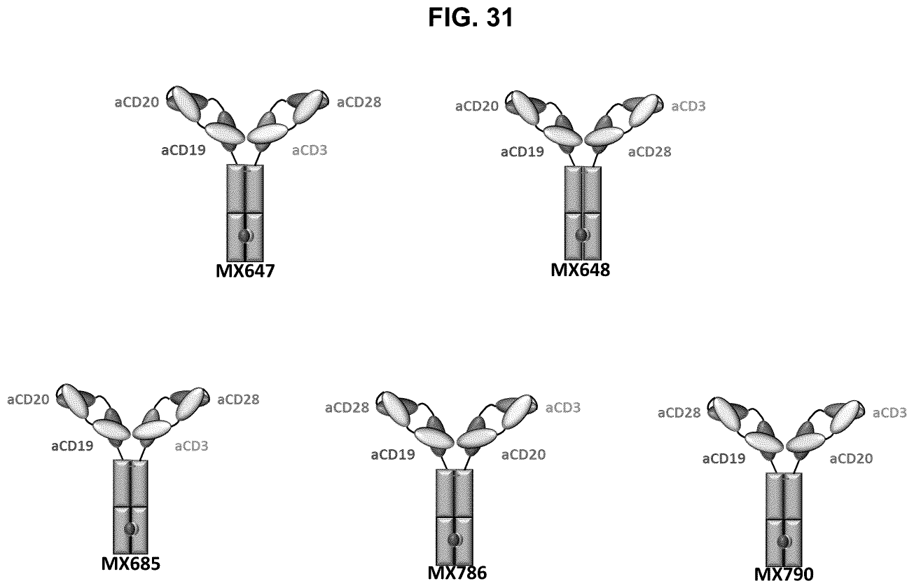

The ability of exemplary non-masked tetraspecific molecules to activate T cells (CD69+), induce in vitro cytolysis, and induce in vitro cytokine release was tested as described above. The molecules tested were MX647, MX648, MX685 (peptide: SEQ ID NO:513 (DNA: SEQ ID NO:514), peptide: SEQ ID NO:591 (DNA: SEQ ID NO:592)), MX786 and MX790 (FIG. 31).

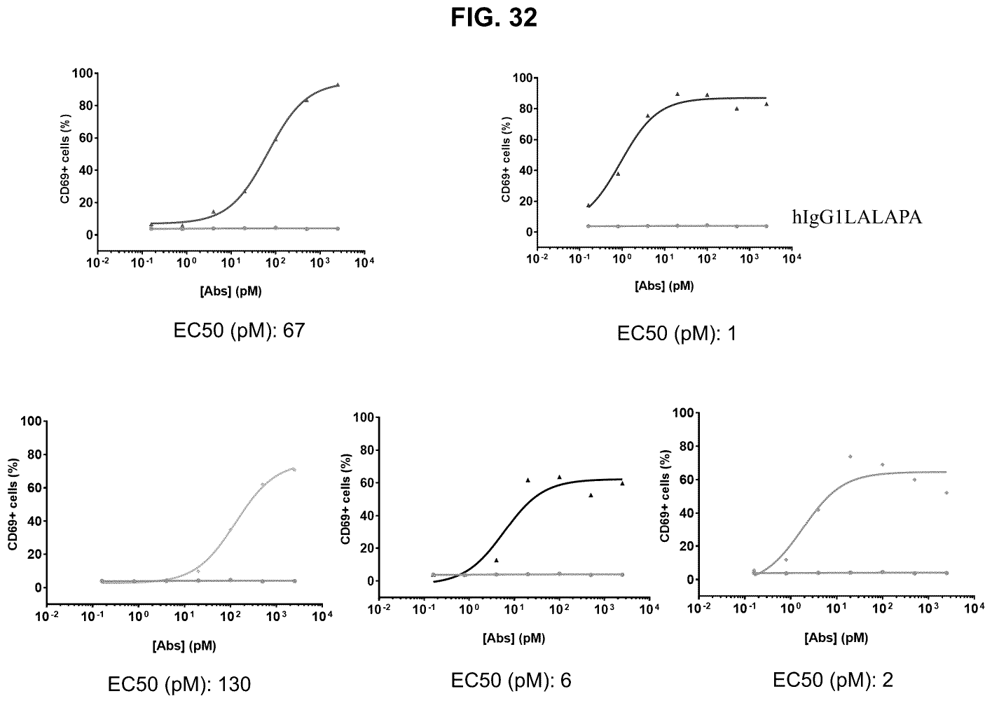

For T cell activation, the percentage of CD69+ cells from PBMCs of a representative donor in the absence of tumor cells and following treatment with MX647, MX648, MX685, MX786 and MX790 are shown in FIG. 32. EC50 in pM and results from a negative control (hIgG1LALAPA) are also shown.

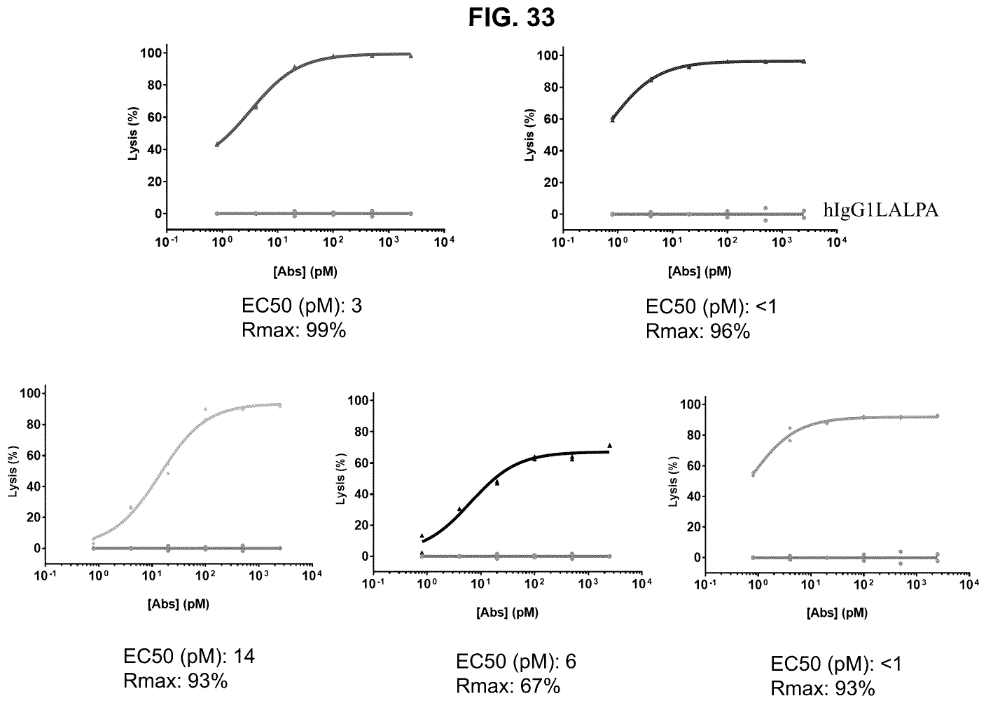

For in vitro cytolysis, cytolysis mediated by MX647, MX648, MX685, MX786 and MX790 or a negative control (hIgG1LALAPA) from PBMCs (E:T: 5:1) was measured from a representative donor. Results are shown in FIG. 33 as percent lysis with increasing concentration of antibody. EC50 in pM and Rmax values are also shown.

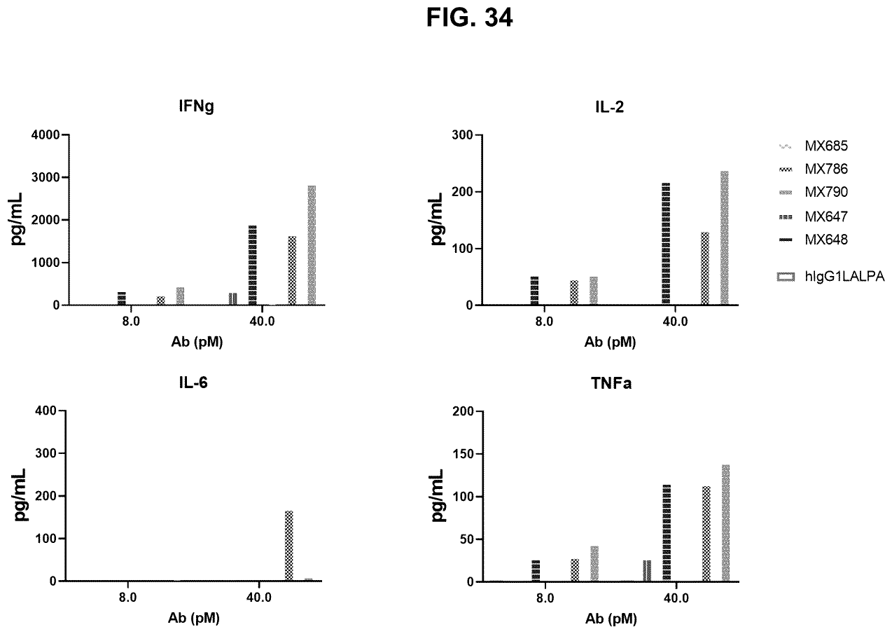

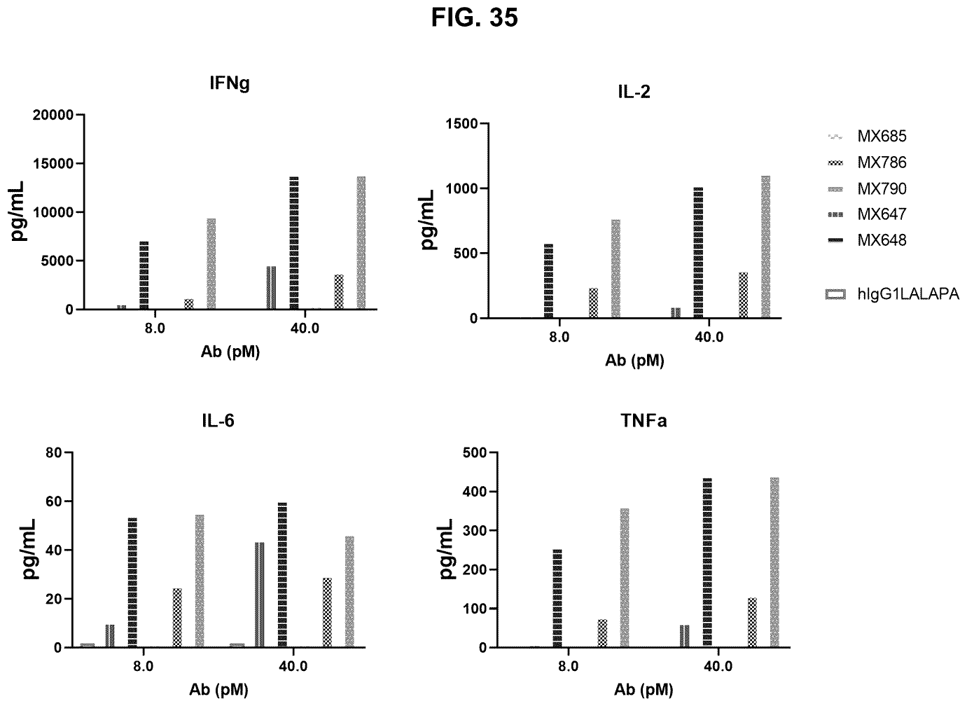

For in vitro cytokine release, IL-2, IFN-gamma, IL-6 and TNFalpha were tested. First, cytokine release from PBMCs from a representative donor in the absence of target tumor cells and following treatment with 8 pM or 40 pM MX647, MX648, MX685, MX786 and MX790 after incubation for 24 hours at 37 C was evaluated. Results are shown in FIG. 34. Results from a negative control (hIgG1LALPA) are also shown. Second, cytokine release from PBMCs from a representative donor in the presence of Z-138 tumor cells and following treatment with 8 pM or 40 pM MX647, MX648, MX685, MX786 and MX790 after incubation for 24 hours at 37 C was evaluated. Results are shown in FIG. 35. Results from a negative control (hIgG1LALAPA) are also shown.

### Example 26

### Surface Staining of Exemplary Trispecific Molecules

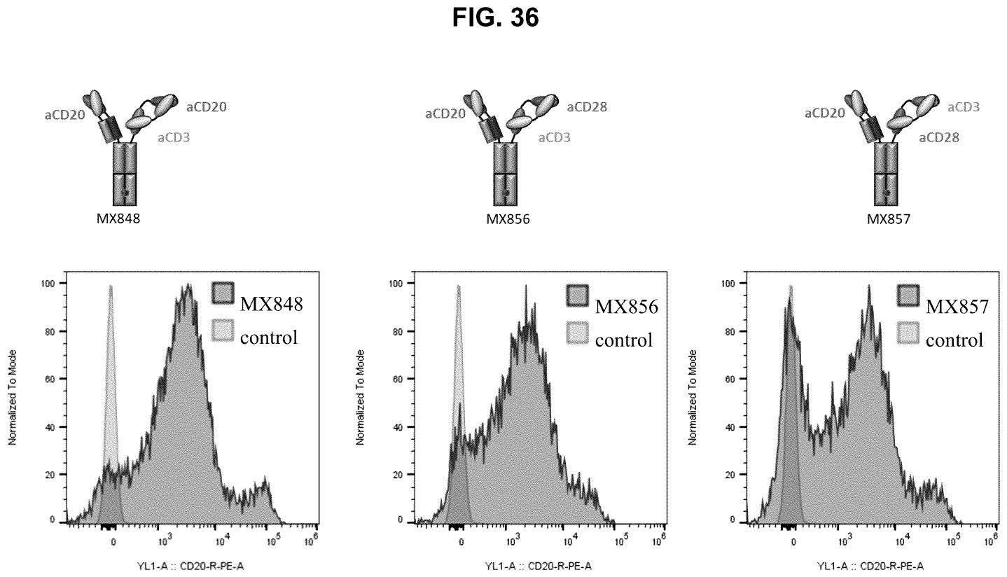

Exemplary trispecific molecules MX848, MX856 and MX857 were prepared as described above and shown in FIG. 36. Surface staining of the molecules to hCD20-expressing Epi293 cells was performed as described above. The results are shown in FIG. 36. Staining with a negative control (control) is also shown.

### Example 27

### In Vitro Cytolysis of Exemplary Trispecific Molecule

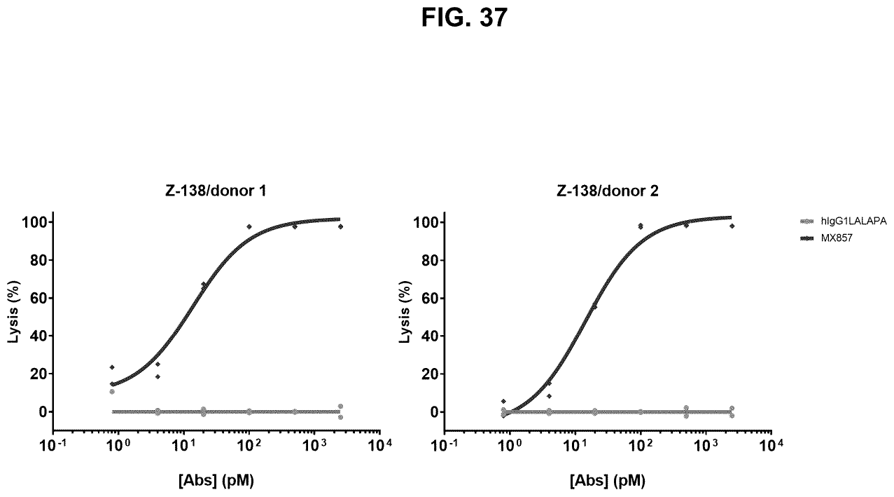

In vitro cytolysis of Z-138 tumor cells mediated by MX857 or a negative control (hIgG1LALAPA) from PBMCs from two representative donors (E:T: 3:1) was measured. Results are shown in FIG. 37 as percent lysis of cells with increasing concentration of antibody.

### Example 28

### Surface Staining of Exemplary Tetraspecific Molecules

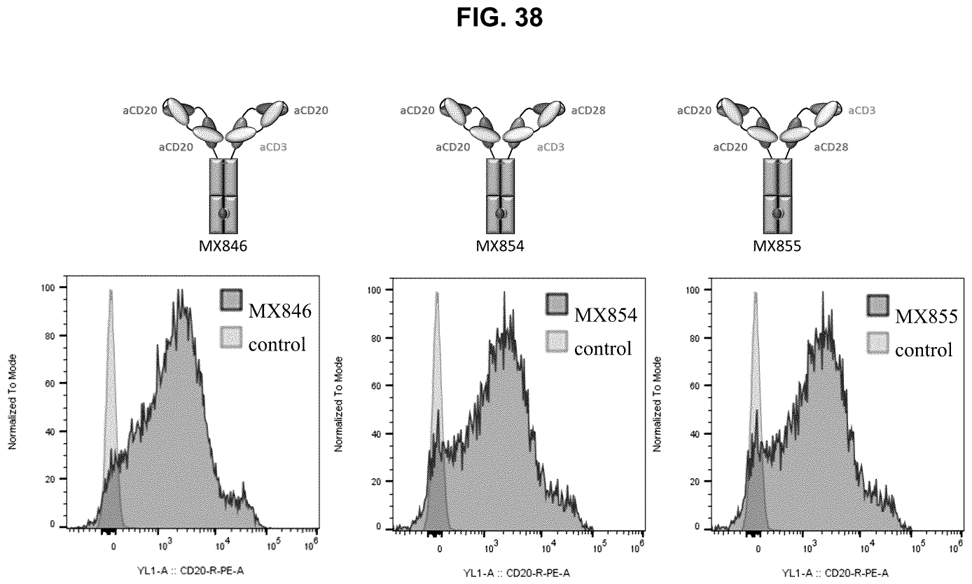

Exemplary tetraspecific molecules MX846, MX854 and MX855 were prepared as described above and shown in FIG. 38. Surface staining of 12.5 nM of the molecules to hCD20-expressing Epi293 cells was performed as described above. The results are shown in FIG. 38. Staining with a negative control (control) is also shown.

### Example 29

### Surface Staining of Exemplary Tetraspecific Molecules

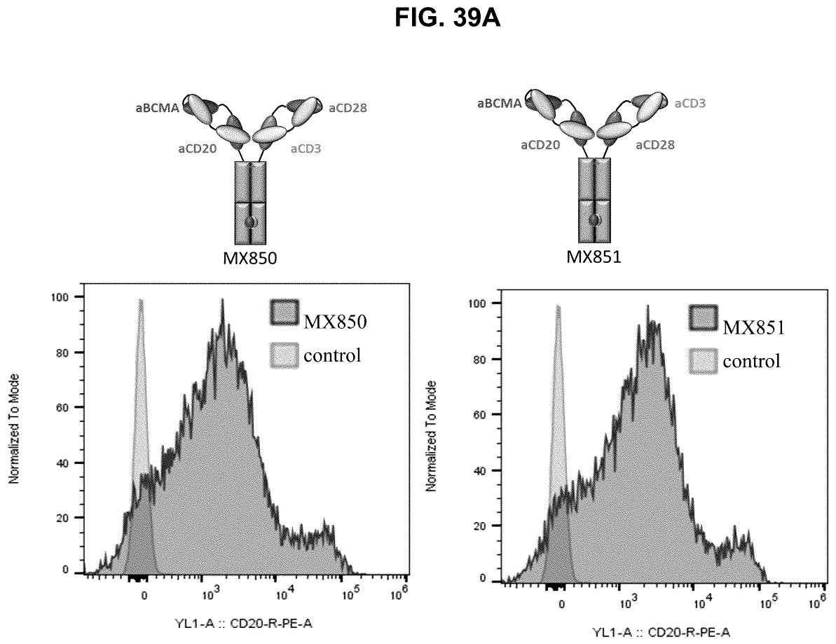

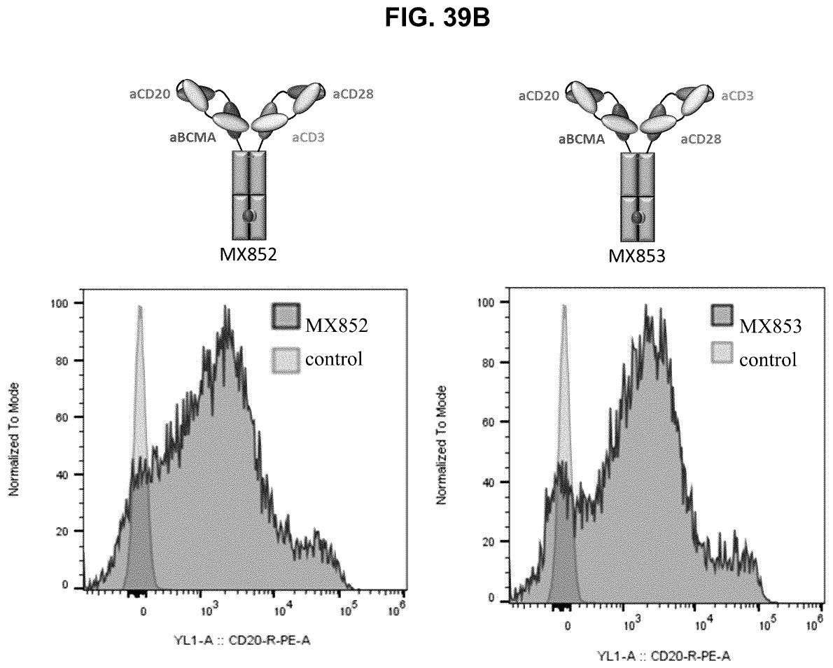

Exemplary tetraspecific molecules MX850, MX851, MX852 and MX853 were prepared as described above and shown in FIG. 39A-39B. Surface staining of 12.5 nM of the molecules to hCD20-expressing Epi293 cells was performed as described above. The results are shown in FIG. 39A-39B. Staining with a negative control (control) is also shown.

### Example 30

### In Vitro Cytolysis of Exemplary Tetraspecific Molecule

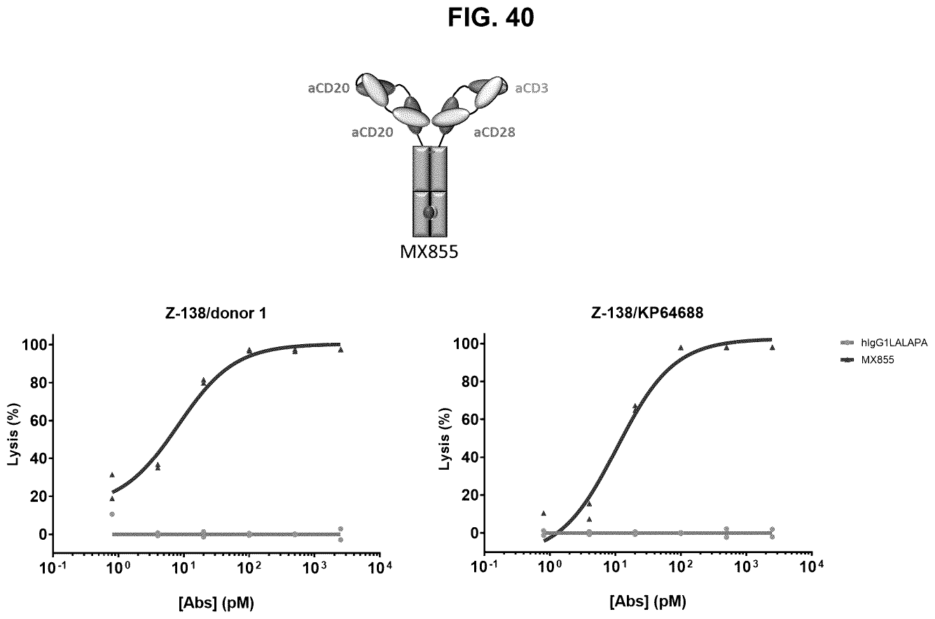

In vitro cytolysis of Z-138 tumor cells mediated by MX855 or a negative control (hIgG1LALAPA) from PBMCs from two representative donors (E:T: 3:1) was measured at 37 C after 40 hours. Results are shown in FIG. 40 as percent lysis of cells with increasing concentration of antibody.

### Example 31

### Binding of MX977 and MX978 to their Targets CD20, CD19, CD28 and CD3 as Measured by Biolayer-Interferometry

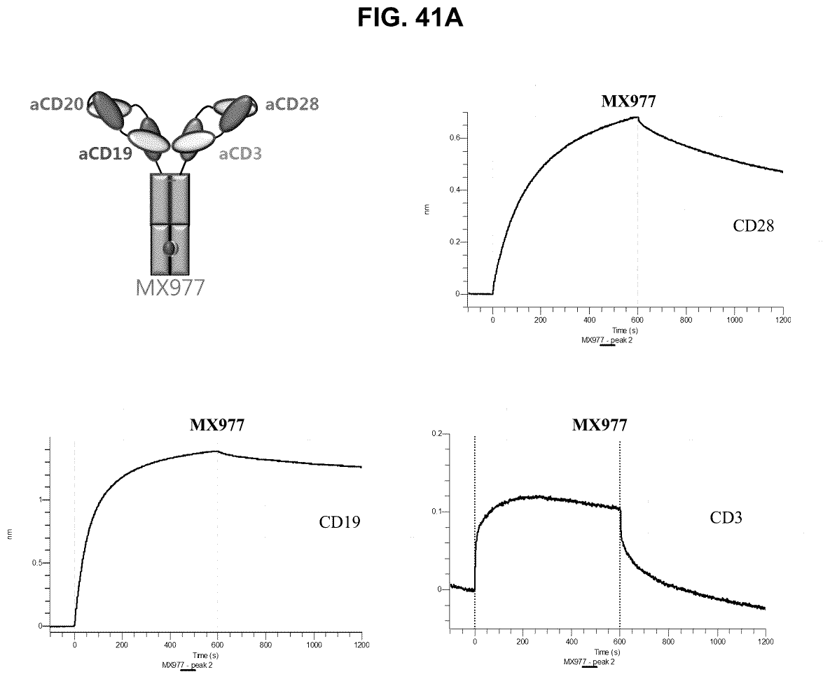

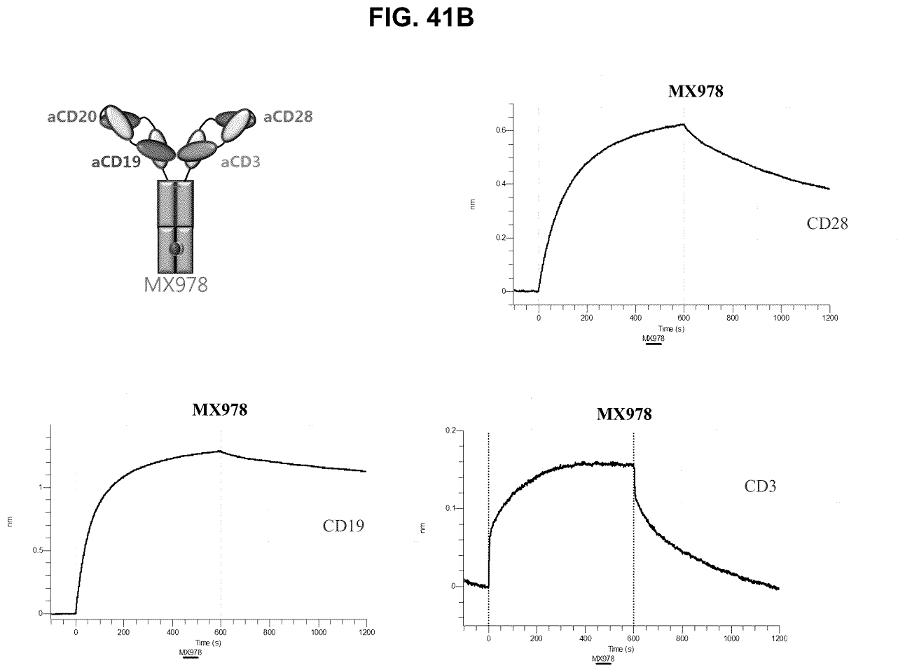

Exemplary tetraspecific molecules MX977 and MX978 were prepared as described above and shown in FIG. 41A-41B. Biolayer-interferometry was performed as described above. Results are shown in FIG. 41A-41B. These results show that MX977 and MX978 bound to their expected targets CD20, CD19, CD28 and CD3.

### Example 32

### Surface Staining of Exemplary Tetraspecific Molecules

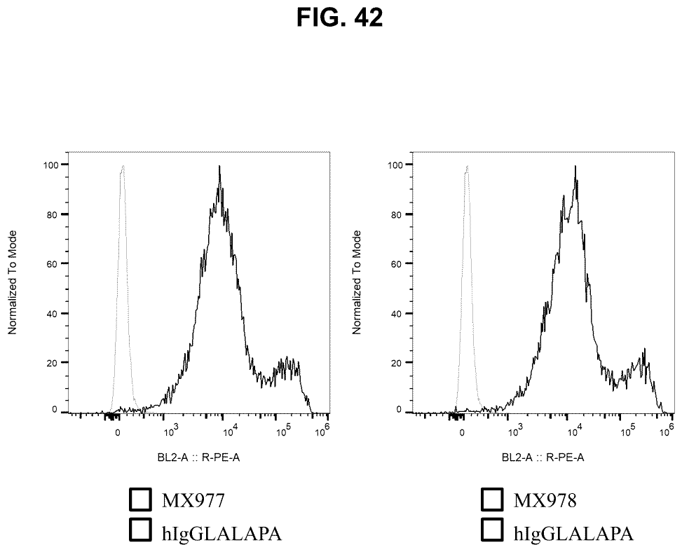

Surface staining of 12.5 nM MX977 and MX978 to hCD20-expressing Epi293 cells was performed as described above. The results are shown in FIG. 42. Staining with a negative control (hIgGLALAPA) is also shown.

### Example 33

### In Vitro Cytolysis of Exemplary Tetraspecific Molecule

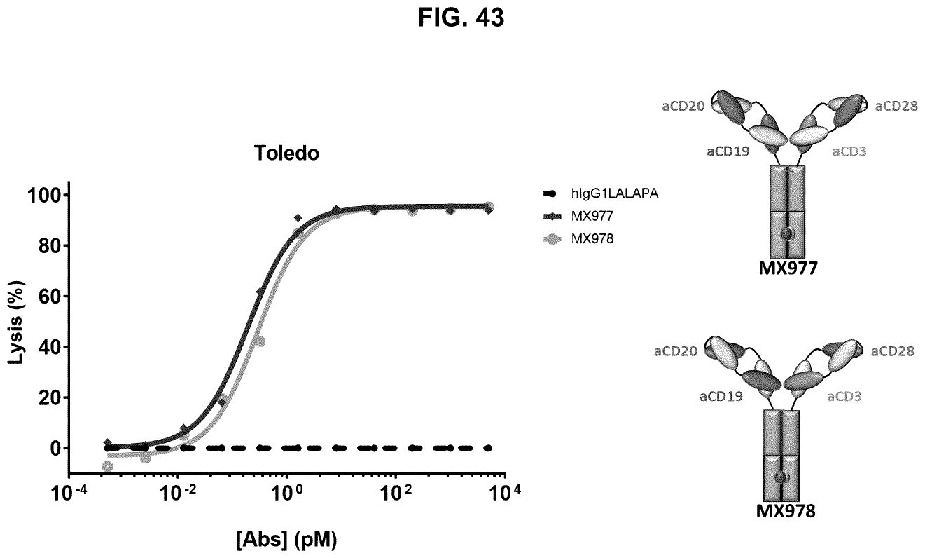

In vitro cytolysis of Toledo tumor cells mediated by MX977, MX978 or a negative control (hIgG1LALAPA) from PBMCs from a representative donor (E:T: 5:1) was measured at 37 C after 40 hours. Results are shown in FIG. 43 as percent lysis of cells with increasing concentration of antibody.

All publications, patents, patent applications and biological deposit mentioned in this application are herein incorporated in their entirety by reference into the specification, to the same extent as if each individual publication, patent, patent application or biological deposit was specifically and individually indicated to be incorporated herein by reference. In addition, citation or identification of any reference in this application shall not be construed as an admission that such reference is available as prior art to the present invention. To the extent that section headings are used, they should not be construed as necessarily limiting.

1. An antigen binding polypeptide complex comprising a first polypeptide, a second polypeptide, and a third polypeptide;
wherein the first polypeptide has a structure represented by:
VL1-L1-VH1-L2-Fc;
VH1-L3-VL1-L4-Fc;
VH1-L1-VL1-L2-Fc; or
VL1-L3-VH1-L4-Fc;
wherein the second polypeptide has a structure represented by:
VH2-L5-VH3-L6-CH1-L7-Fc; or
VH3-L5-VH2-L6-CH1-L7-Fc;
wherein the third polypeptide has a structure represented by:
VL2-L8-VL3-L9-CL; or
VL3-L8-VL2-L9-CL;
wherein
VL1 is a first immunoglobulin light chain variable region;
VL2 is a second immunoglobulin light chain variable region;
VL3 is a third immunoglobulin light chain variable region;
VH1 is a first immunoglobulin heavy chain variable region;
VH2 is a second immunoglobulin heavy chain variable region;
VH3 is a third immunoglobulin heavy chain variable region;
Fc is a region comprising an immunoglobulin heavy chain constant region 2 (CH2), an immunoglobulin heavy chain constant region 3 (CH3), and optionally, an immunoglobulin hinge;
CH1 is an immunoglobulin heavy chain constant region 1;
CL is an immunoglobulin light chain constant region; and
L1-L9 are amino acid linkers.

2. An antigen binding polypeptide complex according to claim 1 comprising a first polypeptide, a second polypeptide, and a third polypeptide;
wherein the first polypeptide has a structure represented by:
VL1-L1-VH1-L2-Fc; or
VH1-L3-VL1-L4-Fc;
wherein the second polypeptide has a structure represented by:
VH2-L5-VH3-L6-CH1-L7-Fc;
wherein the third polypeptide has a structure represented by:
VL2-L8-VL3-L9-CL;
wherein:
VL1 is a first immunoglobulin light chain variable region;
VL2 is a second immunoglobulin light chain variable region;
VL3 is a third immunoglobulin light chain variable region;
VH1 is a first immunoglobulin heavy chain variable region;
VH2 is a second immunoglobulin heavy chain variable region;
VH3 is a third immunoglobulin heavy chain variable region;
Fc is a region comprising an immunoglobulin heavy chain constant region 2 (CH2), an immunoglobulin heavy chain constant region 3 (CH3), and optionally, an immunoglobulin hinge;
CH1 is an immunoglobulin heavy chain constant region 1;
CL is an immunoglobulin light chain constant region; and
L1-L9 are amino acid linkers.

3. An antigen binding polypeptide complex comprising a first polypeptide, a second polypeptide, a third polypeptide, and a fourth polypeptide;
wherein the first polypeptide has a structure represented by:
VL1-L1-CL; or
VH1-L1-CL;
wherein the second polypeptide has a structure represented by:
VH1-L2-CH1-L3-Fc; or
VL1-L2-CH1-L3-Fc;
wherein the third polypeptide has a structure represented by:
VH2-L4-VH3-L5-CH1-L6-Fc; or
VH3-L4-VH2-L5-CH1-L6-Fc;
wherein the fourth polypeptide has a structure represented by:
VL2-L7-VL3-L8-CL; or
VL3-L7-VL2-L8-CL;
wherein:
VL1 is a first immunoglobulin light chain variable region;
VL2 is a second immunoglobulin light chain variable region;
VL3 is a third immunoglobulin light chain variable region;
VH1 is a first immunoglobulin heavy chain variable region;
VH2 is a second immunoglobulin heavy chain variable region;
VH3 is a third immunoglobulin heavy chain variable region;
Fc is a region comprising an immunoglobulin heavy chain constant region 2 (CH2), an immunoglobulin heavy chain constant region 3 (CH3), and optionally, an immunoglobulin hinge;
CH1 is an immunoglobulin heavy chain constant region 1;
CL is an immunoglobulin light chain constant region; and
L1-L8 are amino acid linkers.

4. An antigen binding polypeptide complex according to claim 3 comprising a first polypeptide, a second polypeptide, a third polypeptide, and a fourth polypeptide;
wherein the first polypeptide has a structure represented by:
VL1-L1-CL;
wherein the second polypeptide has a structure represented by:
VH1-L2-CH1-L3-Fc;
wherein the third polypeptide has a structure represented by:
VH2-L4-VH3-L5-CH1-L6-Fc;
wherein the fourth polypeptide has a structure represented by:
VL2-L7-VL3-L8-CL;
wherein:
VL1 is a first immunoglobulin light chain variable region;
VL2 is a second immunoglobulin light chain variable region;
VL3 is a third immunoglobulin light chain variable region;
VH1 is a first immunoglobulin heavy chain variable region;
VH2 is a second immunoglobulin heavy chain variable region;
VH3 is a third immunoglobulin heavy chain variable region;
Fc is a region comprising an immunoglobulin heavy chain constant region 2 (CH2), an immunoglobulin heavy chain constant region 3 (CH3), and optionally, an immunoglobulin hinge;
CH1 is an immunoglobulin heavy chain constant region 1;
CL is an immunoglobulin light chain constant region; and
L1-L8 are amino acid linkers.

5. An antigen binding polypeptide complex comprising a first polypeptide, a second polypeptide, and a third polypeptide;
wherein the first polypeptide has a structure represented by:
VL1-L1-VL2-L2-VH2-L3-VH1-L4-Fc;
VL1-L1-VH2-L2-VL2-L3-VH1-L4-Fc;
VH1-L5-VH2-L6-VL2-L7-VL1-L8-Fc; or
VH1-L5-VL2-L6-VH2-L7-VL1-L8-Fc;
wherein the second polypeptide has a structure represented by:
VH3-L9-VH4-L10-CH1-L11-Fc; or
VH4-L9-VH3-L10-CH1-L11-Fc;
wherein the third polypeptide has a structure represented by:
VL3-L12-VL4-L13-CL; or
VL4-L12-VL3-L13-CL
wherein:
VL1 is a first immunoglobulin light chain variable region;
VL2 is a second immunoglobulin light chain variable region;
VL3 is a third immunoglobulin light chain variable region;
VL4 is a fourth immunoglobulin light chain variable region;
VH1 is a first immunoglobulin heavy chain variable region;
VH2 is a second immunoglobulin heavy chain variable region;
VH3 is a third immunoglobulin heavy chain variable region;
VH4 is a fourth immunoglobulin heavy chain variable region;
Fc is a region comprising an immunoglobulin heavy chain constant region 2 (CH2), an immunoglobulin heavy chain constant region 3 (CH3), and optionally, an immunoglobulin hinge;
CH1 is an immunoglobulin heavy chain constant region 1;
CL is an immunoglobulin light chain constant region; and
L1-L13 are amino acid linkers;
or
wherein the first polypeptide has a structure represented by:
VL1-L1-VL2-L2-VH2-L3-VH1-L4-Fc;
VL1-L1-VH2-L2-VL2-L3-VH1-L4-Fc;
VH1-L5-VH2-L6-VL2-L7-VL1-L8-Fc; or
VH1-L5-VL2-L6-VH2-L7-VL1-L8-Fc;
wherein the second polypeptide has a structure represented by:
VH3-L9-VH4-CH1-Fc; or
VH4-L9-VH3-CH1-Fc;
wherein the third polypeptide has a structure represented by:
VL3-L12-VL4-CL; or
VL4-L12-VL3-CL
wherein:
VL1 is a first immunoglobulin light chain variable region;
VL2 is a second immunoglobulin light chain variable region;
VL3 is a third immunoglobulin light chain variable region;
VL4 is a fourth immunoglobulin light chain variable region;
VH1 is a first immunoglobulin heavy chain variable region;
VH2 is a second immunoglobulin heavy chain variable region;
VH3 is a third immunoglobulin heavy chain variable region;
VH4 is a fourth immunoglobulin heavy chain variable region;
Fc is a region comprising an immunoglobulin heavy chain constant region 2 (CH2), an immunoglobulin heavy chain constant region 3 (CH3), and optionally, an immunoglobulin hinge;
CH1 is an immunoglobulin heavy chain constant region 1;
CL is an immunoglobulin light chain constant region; and
L1-L10 are amino acid linkers.

6. An antigen binding polypeptide complex according to claim 5 comprising a first polypeptide, a second polypeptide, and a third polypeptide;
wherein the first polypeptide has a structure represented by:
VL1-L1-VL2-L2-VH2-L3-VH1-L4-Fc; or
VH1-L5-VH2-L6-VL2-L7-VL1-L8-Fc;
wherein the second polypeptide has a structure represented by:
VH3-L9-VH4-L10-CH1-L11-Fc;
wherein the third polypeptide has a structure represented by:
VL3-L12-VL4-L13-CL;
wherein:
VL1 is a first immunoglobulin light chain variable region;
VL2 is a second immunoglobulin light chain variable region;
VL3 is a third immunoglobulin light chain variable region;
VL4 is a fourth immunoglobulin light chain variable region;
VH1 is a first immunoglobulin heavy chain variable region;
VH2 is a second immunoglobulin heavy chain variable region;
VH3 is a third immunoglobulin heavy chain variable region;
VH4 is a fourth immunoglobulin heavy chain variable region;
Fc is a region comprising an immunoglobulin heavy chain constant region 2 (CH2), an immunoglobulin heavy chain constant region 3 (CH3), and optionally, an immunoglobulin hinge;
CH1 is an immunoglobulin heavy chain constant region 1;
CL is an immunoglobulin light chain constant region; and
L1-L13 are amino acid linkers;
or
wherein the first polypeptide has a structure represented by:
VL1-L1-VL2-L2-VH2-L3-VH1-L4-Fc; or
VH1-L5-VH2-L6-VL2-L7-VL1-L8-Fc;
wherein the second polypeptide has a structure represented by:
VH3-L9-VH4-CH1-Fc;
wherein the third polypeptide has a structure represented by:
VL3-L10-VL4-CL;
wherein:
VL1 is a first immunoglobulin light chain variable region;
VL2 is a second immunoglobulin light chain variable region;
VL3 is a third immunoglobulin light chain variable region;
VL4 is a fourth immunoglobulin light chain variable region;
VH1 is a first immunoglobulin heavy chain variable region;
VH2 is a second immunoglobulin heavy chain variable region;
VH3 is a third immunoglobulin heavy chain variable region;
VH4 is a fourth immunoglobulin heavy chain variable region;
Fc is a region comprising an immunoglobulin heavy chain constant region 2 (CH2), an immunoglobulin heavy chain constant region 3 (CH3), and optionally, an immunoglobulin hinge;
CH1 is an immunoglobulin heavy chain constant region 1;
CL is an immunoglobulin light chain constant region; and
L1-L10 are amino acid linkers.

7. An antigen binding polypeptide complex comprising a first polypeptide and a second polypeptide;
wherein the first polypeptide has a structure represented by:
VL1-L1-VL2-L2-VH2-L3-VH1-L4-Fc;
VL1-L1-VH2-L2-VL2-L3-VH1-L4-Fc;
VH1-L5-VH2-L6-VL2-L7-VL1-L8-Fc; or
VH1-L5-VL2-L6-VH2-L7-VL1-L8-Fc;
wherein the second polypeptide has a structure represented by:
VL3-L9-VL4-L10-VH4-L11-VH3-L12-Fc;
VL3-L9-VH4-L10-VL4-L11-VH3-L12-Fc;
VH3-L13-VH4-L14-VL4-L15-VL3-L16-Fc; or
VH3-L13-VL4-L14-VH4-L15-VL3-L16-Fc;
wherein:
VL1 is a first immunoglobulin light chain variable region that specifically binds to a tumor-associated antigen;
VL2 is a second immunoglobulin light chain variable region that specifically binds to a tumor-associated antigen;
VL3 is a third immunoglobulin light chain variable region that specifically binds to CD3;
VL4 is a fourth immunoglobulin light chain variable region that specifically binds to a tumor-associated antigen;
VH1 is a first immunoglobulin heavy chain variable region that specifically binds to a tumor-associated antigen;
VH2 is a second immunoglobulin heavy chain variable region that specifically binds to a tumor-associated antigen;
VH3 is a third immunoglobulin heavy chain variable region that specifically binds to CD3;
VH4 is a fourth immunoglobulin heavy chain variable region that specifically binds to a tumor-associated antigen;
Fc is a region comprising an immunoglobulin heavy chain constant region 2 (CH2), an immunoglobulin heavy chain constant region 3 (CH3), and optionally, an immunoglobulin hinge; and
L1-L16 are amino acid linkers.

8. An antigen binding polypeptide complex according to claim 7 comprising a first polypeptide and a second polypeptide;
wherein the first polypeptide has a structure represented by:
VL1-L1-VL2-L2-VH2-L3-VH1-L4-Fc; or
VH1-L5-VH2-L6-VL2-L7-VL1-L8-Fc;
wherein the second polypeptide has a structure represented by:
VL3-L9-VL4-L10-VH4-L11-VH3-L12-Fc; or
VH3-L13-VH4-L14-VL4-L15-VL3-L16-Fc;
wherein:
VL1 is a first immunoglobulin light chain variable region that specifically binds to a tumor-associated antigen;
VL2 is a second immunoglobulin light chain variable region that specifically binds to a tumor-associated antigen;
VL3 is a third immunoglobulin light chain variable region that specifically binds to CD3;
VL4 is a fourth immunoglobulin light chain variable region that specifically binds to a tumor-associated antigen;
VH1 is a first immunoglobulin heavy chain variable region that specifically binds to a tumor-associated antigen;
VH2 is a second immunoglobulin heavy chain variable region that specifically binds to a tumor-associated antigen;
VH3 is a third immunoglobulin heavy chain variable region that specifically binds to CD3;
VH4 is a fourth immunoglobulin heavy chain variable region that specifically binds to a tumor-associated antigen;
Fc is a region comprising an immunoglobulin heavy chain constant region 2 (CH2), an immunoglobulin heavy chain constant region 3 (CH3), and optionally, an immunoglobulin hinge; and
L1-L16 are amino acid linkers.

9. An antigen binding polypeptide complex comprising a first polypeptide and a second polypeptide;
wherein the first polypeptide has a structure represented by:
VL1-L1-VL2-L2-VH2-L3-VH1-L4-CH1-L5-Fc;
VL1-L1-VH2-L2-VL2-L3-VH1-L4-CH1-L5-Fc;
VH1-L6-VH2-L7-VL2-L8-VL1-L9-CH1-L10-Fc;
VH1-L6-VL2-L7-VH2-L8-VL1-L9-CH1-L10-Fc;
VL1-L11-VL2-L12-VH2-L13-VH1-L14-CL-L15-Fc;
VL1-L11-VH2-L12-VL2-L13-VH1-L14-CL-L15-Fc;
VH1-L16-VH2-L17-VL2-L18-VL1-L19-CL-L20-Fc;
VH1-L16-VL2-L17-VH2-L18-VL1-L19-CL-L20-Fc;
VL1-L21-VL2-L22-VH2-L23-VH1-L24-CH1-L25-CL-L26-Fc;
VL1-L21-VH2-L22-VL2-L23-VH1-L24-CH1-L25-CL-L26-Fc;
VH1-L27-VH2-L28-VL2-L29-VL1-L30-CH1-L31-CL-L32-Fc;
VH1-L27-VL2-L28-VH2-L29-VL1-L30-CH1-L31-CL-L32-Fc;
VL1-L33-VL2-L34-VH2-L35-VH1-L36-CL-L37-CH1-L38-Fc;
VL1-L33-VH2-L34-VL2-L35-VH1-L36-CL-L37-CH1-L38-Fc;
VH1-L39-VH2-L40-VL2-L41-VL1-L42-CL-L43-CH1-L44-Fc; or
VH1-L39-VL2-L40-VH2-L41-VL1-L42-CL-L43-CH1-L44-Fc;
wherein the second polypeptide has a structure represented by:
VL3-L45-VL4-L46-VH4-L47-VH3-L48-CH1-L49-Fc;
VL3-L45-VH4-L46-VL4-L47-VH3-L48-CH1-L49-Fc;
VH3-L50-VH4-L51-VL4-L52-VL3-L53-CH1-L54-Fc;
VH3-L50-VL4-L51-VH4-L52-VL3-L53-CH1-L54-Fc;
VL3-L55-VL4-L56-VH4-L57-VH3-L58-CL-L59-Fc;
VL3-L55-VH4-L56-VL4-L57-VH3-L58-CL-L59-Fc;
VH3-L60-VH4-L61-VL4-L62-VL3-L63-CL-L64-Fc;
VH3-L60-VL4-L61-VH4-L62-VL3-L63-CL-L64-Fc;
VL3-L65-VL4-L66-VH4-L67-VH3-L68-CH1-L69-CL-L70-Fc;
VL3-L65-VH4-L66-VL4-L67-VH3-L68-CH1-L69-CL-L70-Fc;
VH3-L71-VH4-L72-VL4-L73-VL3-L74-CH1-L75-CL-L76-Fc;
VH3-L71-VL4-L72-VH4-L73-VL3-L74-CH1-L75-CL-L76-Fc;
VL3-L77-VL4-L78-VH4-L79-VH3-L80-CL-L81-CH1-L82-Fc;
VL3-L77-VH4-L78-VL4-L79-VH3-L80-CL-L81-CH1-L82-Fc;
VH3-L83-VH4-L84-VL4-L85-VL3-L86-CL-L87-CH1-L88-Fc; or
VH3-L83-VL4-L84-VH4-L85-VL3-L86-CL-L87-CH1-L88-Fc;
wherein:
VL1 is a first immunoglobulin light chain variable region that specifically binds to a tumor-associated antigen;
VL2 is a second immunoglobulin light chain variable region that specifically binds to a tumor-associated antigen;
VL3 is a third immunoglobulin light chain variable region that specifically binds to CD3;
VL4 is a fourth immunoglobulin light chain variable region that specifically binds to a tumor-associated antigen;
VH1 is a first immunoglobulin heavy chain variable region that specifically binds to a tumor-associated antigen;
VH2 is a second immunoglobulin heavy chain variable region that specifically binds to a tumor-associated antigen;
VH3 is a third immunoglobulin heavy chain variable region that specifically binds to CD3;
VH4 is a fourth immunoglobulin heavy chain variable region that specifically binds to a tumor-associated antigen;
Fc is a region comprising an immunoglobulin heavy chain constant region 2 (CH2), an immunoglobulin heavy chain constant region 3 (CH3), and optionally, an immunoglobulin hinge;
CH1 is an immunoglobulin heavy chain constant region 1;
CL is an immunoglobulin light chain constant region; and
L1-L88 are amino acid linkers.

10. An antigen binding polypeptide complex according to claim 9 comprising a first polypeptide and a second polypeptide;
wherein the first polypeptide has a structure represented by:
VL1-L1-VL2-L2-VH2-L3-VH1-L4-CH1-L5-Fc;
VH1-L6-VH2-L7-VL2-L8-VL1-L9-CH1-L10-Fc;
VL1-L11-VL2-L12-VH2-L13-VH1-L14-CL-L15-Fc;
VH1-L16-VH2-L17-VL2-L18-VL1-L19-CL-L20-Fc;
VL1-L21-VL2-L22-VH2-L23-VH1-L24-CH1-L25-CL-L26-Fc;
VH1-L27-VH2-L28-VL2-L29-VL1-L30-CH1-L31-CL-L32-Fc;
VL1-L33-VL2-L34-VH2-L35-VH1-L36-CL-L37-CH1-L38-Fc; or
VH1-L39-VH2-L40-VL2-L41-VL1-L42-CL-L43-CH1-L44-Fc;
wherein the second polypeptide has a structure represented by:
VL3-L45-VL4-L46-VH4-L47-VH3-L48-CH1-L49-Fc;
VH3-L50-VH4-L51-VL4-L52-VL3-L53-CH1-L54-Fc;
VL3-L55-VL4-L56-VH4-L57-VH3-L58-CL-L59-Fc;
VH3-L60-VH4-L61-VL4-L62-VL3-L63-CL-L64-Fc;
VL3-L65-VL4-L66-VH4-L67-VH3-L68-CH1-L69-CL-L70-Fc;
VH3-L71-VH4-L72-VL4-L73-VL3-L74-CH1-L75-CL-L76-Fc;
VL3-L77-VL4-L78-VH4-L79-VH3-L80-CL-L81-CH1-L82-Fc; or
VH3-L83-VH4-L84-VL4-L85-VL3-L86-CL-L87-CH1-L88-Fc;
wherein:
VL1 is a first immunoglobulin light chain variable region that specifically binds to a tumor-associated antigen;
VL2 is a second immunoglobulin light chain variable region that specifically binds to a tumor-associated antigen;
VL3 is a third immunoglobulin light chain variable region that specifically binds to CD3;
VL4 is a fourth immunoglobulin light chain variable region that specifically binds to a tumor-associated antigen;
VH1 is a first immunoglobulin heavy chain variable region that specifically binds to a tumor-associated antigen;
VH2 is a second immunoglobulin heavy chain variable region that specifically binds to a tumor-associated antigen;
VH3 is a third immunoglobulin heavy chain variable region that specifically binds to CD3;
VH4 is a fourth immunoglobulin heavy chain variable region that specifically binds to a tumor-associated antigen;
Fc is a region comprising an immunoglobulin heavy chain constant region 2 (CH2), an immunoglobulin heavy chain constant region 3 (CH3), and optionally, an immunoglobulin hinge;
CH1 is an immunoglobulin heavy chain constant region 1;
CL is an immunoglobulin light chain constant region; and
L1-L88 are amino acid linkers.

11. The antigen binding polypeptide complex of claim 7, wherein one or more linkers L1-L16 has a length of from 0 amino acids to about 50 amino acids.

12-13. (canceled)

14. The antigen binding polypeptide complex of claim 7, wherein one or more linkers L1-L16 is a cleavable linker.

15. The antigen binding polypeptide complex of claim 14, wherein the cleavable linker is cleaved by a matrix metalloprotease (MMP), a type II transmembrane serine protease, or a MMP and a type II transmembrane serine protease.

16-19. (canceled)

20. The antigen binding polypeptide complex of claim 14, wherein the cleavable linker is GPAALV, GSGRKG, GPLGLTG, GPSGLVG, GLVGRKAG, GPAGLVG, GPAGLVSG, STRKAGG, ASTRKAG, or ASTRKAGG (SEQ ID NOs:22-31), or a sequence having at least 50%, at least 60%, at least 70%, at least 80%, at least 90%, or at least 95% identity to any one of SEQ ID NOs:22-31.

21. The antigen binding polypeptide complex of claim 7, wherein one or more of linkers L1-L16 comprise the amino acid sequence of g, a, gss, asg, ggssg, gssgs, gtvaa, asggs, astgg, ggsgs, asggsg, ggsgssg, ggsggssgss, sggsgssggs, ggsggsgsgggsasgsg, ggsggsgsggggsasgsg, gggssggggsggsgsggsgs, ggggsggsgsggggsasgsg, gggssggsgsggsgsggsgs, sggssggsgsggsgsggsgssg, gsgssggggsggsgsggsgssg, ggggsgsggsgggssggggsggggsggggsggggsggggs, ggggsggggsggggsggggsggggsggggsggggsggggs, ggggsgsggsgggssggggsggggsggggsggggsggggssss, or ggggsgsggsgggssggggsggggsggggsggggsggggssssgs (SEQ ID NOs:1-21), any one of SEQ ID NOs:444, 567, 576 (GGS) and 607, or a sequence having at least 50%, at least 60%, at least 70%, at least 80%, at least 90%, or at least 95% identity to any one of SEQ ID NOs:1-21 and 444, 567, 576 (GGS) and 607.

22-23. (canceled)

24. The antigen binding polypeptide complex of claim 7, wherein VH1 and VL1, VH2 and VL2, VH3 and VL3, or VH4 and VL4 specifically binds to CD3.

25. The antigen binding polypeptide complex of claim 24,
wherein the VH1, VH2, VH3 or VH4 that specifically binds to CD3 comprises a CDR1 comprising an amino acid sequence having at least 90% identity, at least 95% identity, or 100% identity to any one of SEQ ID NOs:32, 40, 96, 433, 524, 667, 703, 739, 767, 803. 839, 851 and 859; a CDR2 comprising an amino acid sequence having at least 90% identity, at least 95% identity, or 100% identity to any one of SEQ ID NOs:33, 41, 97, 434, 525, 668, 704, 740, 768, 804, 840, 852 and 860; and a CDR3 comprising an amino acid sequence having at least 90% identity, at least 95% identity, or 100% identity to any one of SEQ ID NOs:34, 42, 98, 435, 526, 669, 705, 741, 769, 805, 841, 853 and 861; and
wherein the VL1, VL2, VL3 or VL4 that specifically binds to CD3 comprises a CDR1 comprising an amino acid sequence having at least 90% identity, at least 95% identity, or 100% identity to any one of SEQ ID NOs:35, 44, 99, 446, 456, 466, 476, 520, 663, 699, 735, 763, 799, 835, 855 and 863; a CDR2 comprising an amino acid sequence having at least 90% identity, at least 95% identity, or 100% identity to any one of SEQ ID NOs:36, 45, 100, 447 (DT), 457 (DT), 467 (DT), 477 (DT), 521, 664, 700, 736, 764, 800, 836, 856 and 864; and a CDR3 comprising an amino acid sequence having at least 90% identity, at least 95% identity, or 100% identity to any one of SEQ ID NOs:37, 46, 101, 448, 458, 468, 478, 522, 665, 701, 737, 765, 801, 837, 857 and 865.

26. The antigen binding polypeptide complex of claim 24, wherein the VH1, VH2, VH3 or VH4 that specifically binds to CD3 comprises an amino acid sequence having at least 80%, at least 85%, at least 90%, at least 95%, or 100% identity to any one of SEQ ID NOs:38, 43, 102, 423, 432, 523, 666, 702, 738, 766, 802, 838, 850 and 858, and the VL1, VL2, VL3 or VL4 that specifically binds to CD3 comprises an amino acid sequence having at least 80%, at least 85%, at least 90%, at least 95%, or 100% identity to any one of SEQ ID NOs:39, 47, 103, 422, 445, 455, 465, 475, 519, 662, 698, 734, 762, 798, 834, 854 and 862.

27-28. (canceled)

29. The antigen binding polypeptide complex of claim 7, wherein VH1 and VL1, VH2 and VL2, VH3 and VL3, and/or VH4 and VL4 specifically binds to a TAA.

30. The antigen binding polypeptide complex of claim 29, wherein the TAA is A2AR, APRIL, ATPDase, BAFF, BAFFR, BCMA, BlyS, BTK, BTLA, B7DC, B7H1, B7H2, B7H3, B7H4, B7H5, B7H6, B7H7, B7RP1, B7-4, C3, C5, CCL2, CCL3, CCL4, CCL5, CCL7, CCL8, CCL11, CCL15, CCL17, CCL19, CCL20, CCL21, CCL25, CCR3, CCR4, CD16A, CD19, CD20, CD24, CD27, CD28, CD30, CD38, CD39, CD40, CD40L, CD47, CD52, CD70, CD80, CD86, CD123, CD133, CD137, CD137L, CD160, CD272, CEACAM5, CLEC9, CLEC91, CRTH2, CSF-1, CSF-2, CSF-3, CXCL1, CXCL2, CXCL4, CXCL12, CXCL13, CXCR3, cMet, CTLA4, DLL3, DLL4, DNGR-1, E-cadherin, EGFR, ENTPD1, EpCAM, FCER1, FCER1A, FCER2, FGFR, FLAP, FOLH1, Gi24, GITR, GITRL, GPR5, GP100, GPRC5D, HER2, HER3, ICOSL, ICOS, HHLA2, HMGB1, HVEM, IDO, IFNa, IgE, IGF1R, IL2Rbeta, IL1, ILIA, IL1B, IL1F10, IL2, IL4, IL4Ra, IL5, IL5R, IL6, IL7, IL7Ra, IL8, IL9, IL9R, IL10, rhILlO, IL12, IL13, IL13Ra1, IL13Ra2, IL15, IL17, IL17Rb, IL18, IL22, IL23, IL25, IL7, IL33, IL35, ITGB4, ITK, KIR, LAG3, LAMP1, leptin, LPFS2, MHC class II, MUC-1, MUC-16, NCR3LG1, NKG2D, NKp46, NTPDase-1, OX40, OX40L, PD-1, PD-L1, PD-L2, PROM1, S152, SIRPalpha, SISP1, SLC, SPG64, ST2, STEAP1, STEAP2, Syk kinase, STEAP1, TROP2, TACI, TDO, TGFBETA, T14, TIGIT, TIM3, TLR, TLR2, TLR4, TLR5, TLR9, TMEF1, TNFa, TNFRSF7, Tp55, TREM1, TSLP, TSLPR, TWEAK, VEGF, VISTA, Vstm3, or WUCAM.

31. The antigen binding polypeptide complex of claim 29,
wherein the one or more of VH1, VH2, VH3 or VH4 that specifically binds to a TAA comprises a CDR1 comprising an amino acid sequence having at least 90% identity, at least 95% identity, or 100% identity to any one of SEQ ID NOs:64, 72, 80, 8104, 112, 120, 128, 389, 409, 419, 429, 659, 695, 731, 397, 508, 540, 552, 757, 793, 829, 875, 500, 749, 785, 821, 649, 685, 775, 847, 625, 641, 677, 713 and 867; a CDR2 comprising an amino acid sequence having at least 90% identity, at least 95% identity, or 100% identity to any one of SEQ ID NOs:65, 73, 81, 89, 105, 113, 121, 129, 390, 410, 420, 430, 660, 696, 732, 398, 509, 541, 553, 758, 794, 830, 876, 501, 750, 786, 822, 650, 686, 776, 848, 626, 642, 678, 714 and 868; and a CDR3 comprising an amino acid sequence having at least 90% identity, at least 95% identity, or 100% identity to any one of SEQ ID NOs:66, 74, 82, 90, 106, 114, 122, 130, 391, 411, 421, 431, 661, 697, 733, 399, 510, 542, 554, 759, 795, 831, 877, 502, 751, 787, 823, 651, 687, 777, 849, 627, 643, 679, 715 and 869; and
wherein the one or more of VL1, VL2, VL3 or VL4 that specifically binds to a TAA comprises a CDR1 comprising an amino acid sequence having at least 90% identity, at least 95% identity, or 100% identity to any one of SEQ ID NOs:67, 75, 83, 91, 107, 115, 123, 131, 385, 405, 415, 441, 452, 462, 472, 655, 691, 727, 393, 486, 504, 536, 548, 753, 789, 825, 871, 879, 490, 496, 745, 781, 817, 645, 681, 771, 843, 621, 637, 673, 709 and 871; a CDR2 comprising an amino acid sequence having at least 90% identity, at least 95% identity, or 100% identity to any one of SEQ ID NOs:68, 76, 84, 92, 108, 116, 124, 132, 386 (YTS); 406 (YTS), 416 (YTS), 442 (YTS), 453 (YTS), 463 (YTS), 473 (YTS), 656, 692, 728, 394, 487 (AT), 505 (AT), 537, 549, 754, 790, 826, 872, 880, 491 (DA), 497 (DA), 746, 782, 818, 646, 682, 772, 844, 622, 638, 674, 710 and 872; and a CDR3 comprising an amino acid sequence having at least 90% identity, at least 95% identity, or 100% identity to any one of SEQ ID NOs:69, 77, 85, 93, 109, 117, 125, 133, 387, 407, 417, 443, 454, 464, 474, 657, 693, 729, 395, 488, 506, 538, 550, 755, 791, 827, 873, 881, 492, 498, 747, 783, 819, 647, 683, 773, 845, 623, 639, 675, 711 and 873.

32. The antigen binding polypeptide complex of claim 29, wherein the one or more of VH1, VH2, VH3 or VH4 that specifically binds to a TAA comprises an amino acid sequence having at least 80%, at least 85%, at least 90%, at least 95%, or 100% identity to any one of SEQ ID NOs:70, 78, 86, 94, 102, 110, 118, 126, 134, 388, 408, 418, 428, 584, 658, 694, 730, 396, 403, 507, 539, 551, 756, 792, 883, 828, 866, 874, 499, 544, 556, 748, 784, 820, 528, 648, 684, 774, 846, 624, 640, 676 and 712; and the one or more of VL1, VL2, VL3 or VL4 that specifically binds to a TAA comprises an amino acid sequence having at least 80%, at least 85%, at least 90%, at least 95%, or 100% identity to any one of SEQ ID NOs:71, 79, 87, 95, 103, 111, 119, 127, 135, 384, 404, 414, 440, 451, 461, 471, 583, 654, 690, 726, 392, 402, 485, 503, 535, 547, 752, 788, 824, 882, 870, 878, 489, 495, 543, 555, 744, 780, 816, 527, 644, 680, 770, 842, 620, 636, 672 and 708.

33-34. (canceled)

35. The antigen binding polypeptide complex of claim 7, wherein VH1 and VL1, VH2 and VL2, VH3 and VL3, and/or VH4 and VL4 specifically binds to an immune stimulating receptor.

36. The antigen binding polypeptide complex of claim 35, wherein the immune stimulating receptor is CD28.

37. The antigen binding polypeptide complex of claim 35,
wherein the VH1, VH2, VH3 or VH4 that specifically binds to an immune stimulating receptor comprises a CDR1 comprising an amino acid sequence having at least 90% identity, at least 95% identity, or 100% identity to any one of SEQ ID NOs:48, 56, 649, 685, 775, 847, 721 and 811; a CDR2 comprising an amino acid sequence having at least 90% identity, at least 95% identity, or 100% identity to any one of SEQ ID NOs:49, 57, 650, 686, 776, 848, 722 and 812; and a CDR3 comprising an amino acid sequence having at least 90% identity, at least 95% identity, or 100% identity to any one of SEQ ID NOs:50, 58, 651, 687, 777, 849, 723 and 813; and
wherein the VL1, VL2, VL3 or VL4 that specifically binds to an immune stimulating receptor comprises a CDR1 comprising an amino acid sequence having at least 90% identity, at least 95% identity, or 100% identity to any one of SEQ ID NOs:51, 59, 645, 681, 771, 843, 717 and 807; a CDR2 comprising an amino acid sequence having at least 90% identity, at least 95% identity, or 100% identity to any one of SEQ ID NOs:52, 60, 646, 682, 772, 844, 718 and 808; and a CDR3 comprising an amino acid sequence having at least 90% identity, at least 95% identity, or 100% identity to any one of SEQ ID NOs:53, 61, 647, 683, 773, 845, 719 and 809.

38. The antigen binding polypeptide complex of claim 35, wherein the VH1, VH2, VH3 or VH4 that specifically binds to an immune stimulating receptor comprises an amino acid sequence having at least 80%, at least 85%, at least 90%, at least 95%, or 100% identity to any one of SEQ ID NOs:54, 62, 528, 648, 684, 774 and 846, and the VL1, VL2, VL3 or VL4 that specifically binds to an immune stimulating receptor comprises an amino acid sequence having at least 80%, at least 85%, at least 90%, at least 95%, or 100% identity to any one of SEQ ID NOs:55, 63, 527, 644, 680, 770 and 842.

39. The antigen binding polypeptide complex of claim 1, wherein L5, L8, or L5 and L8 are cleavable linkers.

40. The antigen binding polypeptide complex of claim 39, wherein VH3 and VL3 specifically bind to CD3.

41. The antigen binding polypeptide complex of claim 39, wherein VH1 and VL1 and/or VH2 and VL2 specifically bind to a TAA.

42. The antigen binding polypeptide complex of claim 39, wherein VH1 and VL1 or VH2 and VL2 specifically bind to CD28.

43. The antigen binding polypeptide complex of claim 3, wherein L4, L7, or L4 and L7 are cleavable linkers.

44. The antigen binding polypeptide complex of claim 43, wherein VH3 and VL3 specifically bind to CD3.

45. The antigen binding polypeptide complex of claim 43, wherein VH1 and VL1 and/or VH2 and VL2 specifically bind to a TAA.

46. The antigen binding polypeptide complex of claim 43, wherein VH1 and VL1 or VH2 and VL2 specifically bind to CD28.

47. The antigen binding polypeptide complex of claim 5, wherein L9, L12, or L9 and L12 are cleavable linkers.

48. The antigen binding polypeptide complex of claim 47, wherein VH4 and VL4 specifically bind to CD3.

49. The antigen binding polypeptide complex of claim 47, wherein one or more of VH1 and VL1, VH2 and VL2, and VH3 and VL3 specifically bind to a TAA.

50. The antigen binding polypeptide complex of claim 47, wherein VH1 and VL1 specifically bind to a TAA, VH2 and VL2 specifically bind to a TAA, and VH3 and VL3 specifically bind to CD28.

51. The antigen binding polypeptide complex of claim 47, wherein VH1 and VL1 specifically bind to CD28, VH2 and VL2 specifically bind to a TAA, and VH3 and VL3 specifically bind to a TAA.

52. The antigen binding polypeptide complex of claim 47, wherein VH1 and VL1 specifically bind to cMet, VH2 and VL2 specifically bind to Trop2, and VH3 and VL3 specifically bind to CD28.

53. The antigen binding polypeptide complex of claim 47, wherein VH1 and VL1 specifically bind to Trop2, VH2 and VL2 specifically bind to cMet, and VH3 and VL3 specifically bind to CD28.

54. The antigen binding polypeptide complex of claim 47, wherein VH1 and VL1 specifically bind to cMet, VH2 and VL2 specifically bind to CD28, and VH3 and VL3 specifically bind to Trop2.

55. The antigen binding polypeptide complex of claim 47, wherein VH1 and VL1 specifically bind to CD28, VH2 and VL2 specifically bind to cMet, and VH3 and VL3 specifically bind to Trop2.

56. The antigen binding polypeptide complex of claim 47, wherein VH1 and VL1 specifically bind to Trop2, VH2 and VL2 specifically bind to CD28, and VH3 and VL3 specifically bind to cMet.

57. The antigen binding polypeptide complex of claim 47, wherein VH1 and VL1 specifically bind to CD28, VH2 and VL2 specifically bind to Trop2, and VH3 and VL3 specifically bind to cMet.

58. The antigen binding polypeptide complex of claim 7, wherein L1, L3, or L1 and L3 are cleavable linkers, or L5, L7, or L5 and L7 are cleavable linkers.

59. The antigen binding polypeptide complex of claim 7, wherein L9, L11, or L9 and L11 are cleavable linkers, or L13, L15, or L13 and L15 are cleavable linkers.

60. The antigen binding polypeptide complex of claim 58, wherein VH1 and VL1, VH2 and VL2, VH3 and VL3, or VH4 and VL4 specifically bind to CD3.

61. The antigen binding polypeptide complex of claim 58, wherein one or more of VH1 and VL1, VH2 and VL2, VH3 and VL3, and VH4 and VL4 specifically bind to a TAA.

62. The antigen binding polypeptide complex of claim 58, wherein VH1 and VL1, VH2 and VL2, VH3 and VL3, or VH4 and VL4 specifically bind to CD28.

63. (canceled)

64. The antigen binding polypeptide complex of claim 7, wherein the immunoglobulin hinge comprises an upper hinge region, a middle hinge region, a lower hinge region, or a combination thereof.

65. The antigen binding polypeptide complex of claim 7, wherein the Fc region comprises at least one knob-into-hole modification.

66. The antigen binding polypeptide complex of claim 65, wherein the antigen binding polypeptide complex is an IgG1 or IgG4 antibody and the knob-into-hole modification comprises:
(i) knob substitutions of S354C and T366W and hole substitutions of Y349C, T366S, L368A and Y407V;
(ii) hole substitutions of L234A, L235A and P329A;
(iii) hole substitutions of L234A and L235A;
(iv) hole substitutions of M428L and N433S;
(v) hole substitutions of M252Y, S254T and T256E; or
(vi) a combination thereof;
based on the EU numbering scheme.

67. An antibody or antigen binding fragment thereof comprising the antigen binding polypeptide complex of claim 7.

68. A pharmaceutical composition comprising the antigen binding polypeptide complex of claim 7, and a pharmaceutically acceptable carrier.

69. A kit comprising an antigen binding polypeptide complex of claim 7, and instructions for use.

70. A method for treating cancer, comprising administering to a subject in need thereof the antigen binding polypeptide complex of claim 7.

71-75. (canceled)
